(19) United States
(12) Patent Application Publication
(10) Pub. No.: US 2024/0344070 A1
(43) Pub. Date: Oct. 17, 2024

(54) METHODS AND COMPOSITIONS FOR TREATING SUBJECTS HAVING OR AT RISK OF DEVELOPING A NON-PRIMARY HYPEROXALURIA DISEASE OR DISORDER

(71) Applicant: Alnylam Pharmaceuticals, Inc., Cambridge, MA (US)

(72) Inventors: John M. Gansner, Newton, MA (US); David Erbe, Arlington, MA (US)

(21) Appl. No.: 18/403,862

(22) Filed: Jan. 4, 2024

Related U.S. Application Data
(63) Continuation of application No. PCT/US2022/037453, filed on Jul. 18, 2022.
(60) Provisional application No. 63/223,278, filed on Jul. 19, 2021.

(51) Int. Cl. C12N 15/113

(52) U.S. Cl. CPC C12N 15/1137; C12Y 101/03015; C12N 2310/14; C12N 2310/351

(57) ABSTRACT

The present invention provides methods for treating subjects having or at risk of developing a non-primary hyperoxaluria disease or disorder that would benefit from reduction in oxalate, and compositions comprising nucleic acid inhibitors, e.g., double stranded ribonucleic acid (dsRNA) agents or single stranded antisense polynucleotide agents targeting lactate dehydrogenase A (LDHA), hydroxyacid oxidase (HAO1) and/or proline dehydrogenase 2 (PRODH2), for treating such subjects.

### RELATED APPLICATIONS

This application is a 35 § U.S.C. 111(a) continuation application which claims the benefit of priority to PCT/US2022/037453, filed on Jul. 18, 2022, which, in turn, claims the benefit of priority to U.S. Provisional Application No. 63/223,278, filed on Jul. 19, 2021. The entire contents of each of the foregoing applications are incorporated herein by reference.

### SEQUENCE LISTING

The instant application contains a Sequence Listing which has been submitted electronically in XML file format and is hereby incorporated by reference in its entirety. Said XML copy, created on Jan. 3, 2024, is named 121301_16202_SL.xml and is 33,486,627 bytes in size.

### BACKGROUND OF THE INVENTION

Oxalate (C₂O₄²⁻) is the salt-forming ion of oxalic acid (C₂H₂O₄) that is widely distributed in both plants and animals. It is an unavoidable component of the human diet and a ubiquitous component of plants and plant-derived foods. Oxalate can also be synthesized endogenously via the metabolic pathways that occur in the liver. Dietary and endogenous contributions to urinary oxalate excretion are equal. Glyoxylate is an immediate precursor to oxalate and is derived from the oxidation of glycolate by the enzyme glycolate oxidase (GO), also known, and referred to herein, as hydroxyacid oxidase (HAO1), or by catabolism of hydroxyproline, a component of collagen, by proline dehydrogenase 2 (PRODH2, also known as HYPDH). Transamination of glyoxylate with alanine by the enzyme alanine-glyoxylate aminotransferase (AGXT) results in the formation of pyruvate and glycine. Excess glyoxylate is converted to oxalate by lactate dehydrogenase A (LDHA). The endogenous pathway for oxalate metabolism is illustrated in FIG. 1.

Since oxalate binds with calcium in the kidney, urinary CaOx supersaturation may occur, resulting in the formation and deposition of CaOx crystals in renal tissue or collecting system, even in the presence of normal levels of oxalate. These CaOx crystals contribute to the formation of diffuse renal calcifications (nephrocalcinosis) and stones (nephrolithiasis). Subjects having diffuse renal calcifications or non-obstructing stones typically have no symptoms. However, obstructing stones can cause severe pain. Moreover, over time, these CaOx crystals cause injury and progressive inflammation to the kidney and, when secondary complications such as obstruction are present, these CaOx crystals may lead to decreased renal function and in severe cases even to end-stage renal failure and the need for dialysis.

Primary hyperoxaluria is a well-known disease associated with high levels of oxalate. Specifically, primary hyperoxaluria is characterized by impaired glyoxylate metabolism resulting in overproduction and accumulation of oxalate throughout the body, typically manifesting as kidney and bladder stones. There are three major types of primary hyperoxaluria that differ in their severity and genetic cause. Autosomal recessive mutations in the AGXT gene cause primary hyperoxaluria type 1 (PH1); autosomal recessive mutations in the GRHPR gene cause primary hyperoxaluria type 2 (PH2); and autosomal recessive mutations in the HOGA1 gene cause primary hyperoxaluria type 3 (PH3) (see, FIG. 1). There are few treatment options for subjects having a hereditary hyperoxaluria. Ultimately, some subjects with hereditary hyperoxaluria develop end stage renal disease (ESRD) and require kidney/liver transplants.

Recently, two investigational therapeutics for the treatment of subjects having PH1 or PH2 that reduce oxalate have entered the clinic. Specifically, Lumasiran, an RNA interference (RNAi) therapeutic targeting glycolate oxidase (GO) for the treatment of PH1 is currently being evaluated in a Phase III clinical trail (see, e.g., NCT03681184), and DCR-PHXC, an RNA interference (RNAi) therapeutic targeting LDHA for the treatment of PH1 and PH2 has entered Phase II clinical trials (see, e.g., NCT03847909).

However, there are a significant number of subjects that do not have primary hyperoxaluria, e.g., PH1, PH2, or PH3, and yet still would benefit from reduction in oxalate, for example, subjects having a non-primary hyperoxaluria disease or disorder, for which no effective treatments currently exist. For example, as indicated above, CaOx crystals can form and be deposited in renal tissue or collecting system, even in the presence of normal levels of oxalate and contribute to the formation of diffuse renal calcifications (nephrocalcinosis) and stones (nephrolithiasis). In the presence of other comorbidities, such as a metabolic disorder, e.g., diabetes, Crohn's disease, or bariatric surgery, subjects having such comorbidities may be at risk of developing, e.g., obstructing stones, progressive inflammation of the kidney, decreased renal function and end-stage renal failure.

Accordingly, there is a need in the art for methods to treat subjects having, or at risk of developing, a non-primary hyperoxaluria that would benefit from treatment with agents that reduce oxalate, such as a nucleic acid inhibitor of lactate dehydrogenase A (LDHA), a nucleic acid inhibitor of proline dehydrogenase 2 (PRODH2) and/or a nucleic acid inhibitor of hydroxyacid oxidase (HAO1).

### SUMMARY OF THE INVENTION

The present invention is based, at least in part, on the discovery that agents that reduce oxalate levels, such as a nucleic acid inhibitor of lactate dehydrogenase A (LDHA), a nucleic acid inhibitor of hydroxyacid oxidase (HAO1) and/or a nucleic acid inhibitor of proline dehydrogenase 2 (PRODH2), can be used to treat subjects having or at risk of developing a non-primary hyperoxaluria disease or disorder, such as a subject having normal urinary oxalate levels, e.g., normal urinary calcium oxalate levels, or elevated urinary oxalate levels, e.g., elevated urinary calcium oxalate levels, e.g., supersaturated urinary calcium oxalate levels, e.g., a subject having a kidney stone disease, e.g., calcium oxalate kidney stone disease, such as recurrent calcium oxalate kidney stone disease.

Accordingly, the present invention provides methods for inhibiting the expression of hydroxyacid oxidase (HAO1) in a subject having a non-primary hyperoxaluria disease or disorder that would benefit from reduction in urinary oxalate, methods for reducing urinary oxalate levels in a subject having a non-primary hyperoxaluria disease or disorder that would benefit from reduction in urinary oxalate, and methods for treating a subject having having or at risk of developing a non-primary hyperoxaluria disease or disorder that would benefit from reduction in oxalate, and compositions comprising nucleic acid inhibitors, e.g., double stranded ribonucleic acid (dsRNA) agents or single stranded antisense polynucleotide agents targeting lactate dehydrogenase A (LDHA), hydroxyacid oxidase (HAO1) and/or proline dehydrogenase 2 (PRODH2).

In one aspect, the present invention provides a method for inhibiting the expression of hydroxyacid oxidase (HAO1) in a subject having a non-primary hyperoxaluria disease or disorder that would benefit from reduction in urinary oxalate, comprising administering to the subject a fixed dose of about 200 mg to about 600 mg of a double stranded ribonucleic acid (dsRNA) agent, or salt thereof, which inhibits the expression of of HAO1, thereby inhibiting the expression of HAO1 in the subject.

In another aspect, the present invention provides a method for reducing urinary oxalate levels in a subject having a non-primary hyperoxaluria disease or disorder that would benefit from reduction in urinary oxalate, comprising administering to the subject a fixed dose of about 200 mg to about 600 mg of a double stranded ribonucleic acid (dsRNA) agent, or salt thereof, which inhibits the expression of of HAO1, thereby reducing urinary oxalate levels in the subject.

In one embodiment, the urinary oxalate is urinary calcium oxalate.

In one embodiment, the reduction in urinary calcium oxalate is reduction in urinary calcium oxalate supersaturation.

In one aspect, the present invention provides a method for treating a subject having a non-primary hyperoxaluria disease or disorder that would benefit from reduction in oxalate, comprising administering to the subject a fixed dose of about 200 mg to about 600 mg of a double stranded ribonucleic acid (dsRNA) agent, or salt thereof, which inhibits the expression of of HAO1, thereby treating the subject having the non-primary hyperoxaluria disease or disorder that would benefit from reduction in oxalate.

In one embodiment, the non-primary hyperoxaluria disease or disorder is selected from the group consisting of secondary hyperoxaluria, a kidney stone disease, chronic kidney disease (CKD), end-stage renal disease (ESRD), coronary artery disease, cutaneous oxalate deposition, ethylene glycol poisoning, planned kidney transplantation, and previous kidney transplantation.

In one embodiment, the non-primary hyperoxaluria disease or disorder is a kidney stone disease.

In one embodiment, the kidney stone disease is calcium oxalate kidney stone disease.

In one embodiment, the calcium oxalate kidney stone disease is recurrent calcium oxalate kidney stone disease.

In one embodiment, administration of the dsRNA agent, or salt thereof, to the subject reduces urinary oxalate levels.

In one embodiment, the urinary oxalate is urinary calcium oxalate.

In one embodiment, the reduction in urinary calcium oxalate is reduction in urinary calcium oxalate supersaturation.

In one embodiment, administration of the dsRNA agent, or salt thereof, to the subject reduces clinical and radiographic kidney stone events.

In one embodiment, the subject is a human.

In one embodiment, the dsRNA agent, or salt thereof, is administered to the subject at an interval of once every six months.

In one embodiment, the dsRNA agent, or salt thereof, is administered to the subject initially, at three months, and every six months thereafter.

In one embodiment, the fixed dose of the dsRNA agent, or salt thereof, is about 284 mg.

In one embodiment, the fixed dose of the dsRNA agent, or salt thereof, is about 567 mg.

In one embodiment, the dsRNA agent, or salt thereof, is administered to the subject subcutaneously.

In one embodiment, the subcutaneous administration is subcutaneous injection.

In one embodiment, the dsRNA agent, or salt thereof, comprises a sense strand and an antisense strand forming a double stranded region, wherein the sense strand comprises a nucleotide sequence comprising at least 15 contiguous nucleotides differing by no more than 3 nucleotides from a portion of the nucleotide sequence of SEQ ID NO: 21 and the antisense strand comprises a nucleotide sequence comprising at least 15 contiguous nucleotides differing by no more than 3 nucleotides from the corresponding portion of nucleotide sequence of SEQ ID NO: 22 such that the sense strand is complementary to the at least 15 contiguous nucleotides in the antisense strand.

In one embodiment, the dsRNA agent, or salt thereof, comprises a sense strand and an antisense strand forming a double stranded region, wherein the antisense strand comprises at least 15 contiguous nucleotides differing by no more than 3 nucleotides from any one of the antisense sequences listed in any one of Tables 4-14.

In one embodiment, the dsRNA agent, or salt thereof, comprises a sense strand and an antisense strand forming a double-stranded region, wherein the sense strand comprises a nucleotide sequence differing by no more than 3 nucleotides from the nucleotide sequence 5′-GACUUUCAUCCUGGAAAUAUA-3′ (SEQ ID NO:33) and the antisense strand comprises a nucleotide sequence differing by no more than 3 nucleotides from the nucleotide sequence 5′-UAUAUUUCCAGGAUGAAAGUCCA-3′ (SEQ ID NO:34).

In one embodiment, the dsRNA agent comprises at least one modified nucleotide.

In one embodiment, no more than five of the sense strand nucleotides and no more than five of the nucleotides of the antisense strand are unmodified nucleotides.

In one embodiment, substantially all of the nucleotides of the sense strand and substantially all of the nucleotides of the antisense strand are modified nucleotides.

In one embodiment, all of the nucleotides of the sense strand and all of the nucleotides of the antisense strand are modified nucleotides.

In one embodiment, at least one of the modified nucleotides is selected from the group a deoxy-nucleotide, a 3′-terminal deoxy-thymine (dT) nucleotide, a 2′-O-methyl modified nucleotide, a 2′-fluoro modified nucleotide, a 2′-deoxy-modified nucleotide, a locked nucleotide, an unlocked nucleotide, a conformationally restricted nucleotide, a constrained ethyl nucleotide, an abasic nucleotide, a 2′-amino-modified nucleotide, a 2′-O-allyl-modified nucleotide, 2′-C-alkyl-modified nucleotide, 2′-hydroxyl-modified nucleotide, a 2′-methoxyethyl modified nucleotide, a 2′-O-alkyl-modified nucleotide, a morpholino nucleotide, a phosphoramidate, a non-natural base comprising nucleotide, a tetrahydropyran modified nucleotide, a 1,5-anhydrohexitol modified nucleotide, a cyclohexenyl modified nucleotide, a nucleotide comprising a 5′-phosphorothioate group, a nucleotide comprising a 5′-methylphosphonate group, a nucleotide comprising a 5′ phosphate or 5′ phosphate mimic, a nucleotide comprising vinyl phosphonate, a nucleotide comprising adenosine-glycol nucleic acid (GNA), a nucleotide comprising thymidine-glycol nucleic acid (GNA) S-Isomer, a nucleotide comprising 2-hydroxymethyl-tetrahydrofurane-5-phosphate, a nucleotide comprising 2′-deoxythymidine-3′phosphate, a nucleotide comprising 2′-deoxyguanosine-3′-phosphate, and a terminal nucleotide linked to a cholesteryl derivative and a dodecanoic acid bisdecylamide group; and combinations thereof.

In one embodiment, the dsRNA agent, or salt thereof, further comprises at least one phosphorothioate internucleotide linkage.

In one embodiment, the phosphorothioate or methylphosphonate internucleotide linkage is at the 3′-terminus of one strand.

In one embodiment, the phosphorothioate or methylphosphonate internucleotide linkage is at the 5′-terminus of one strand.

In one embodiment, the phosphorothioate or methylphosphonate internucleotide linkage is at the both the 5′- and 3′-terminus of one strand In one embodiment, the dsRNA agent, or salt thereof, comprises 6-8 phosphorothioate internucleotide linkages.

In one embodiment, at least one strand of the dsRNA agent, or salt thereof, further comprises a ligand.

In one embodiment, the ligand is attached to the 3′ end of the sense strand.

In one embodiment, the ligand is one or more N-acetylgalactosamine (GalNAc) derivatives.

In one embodiment, the one or more GalNAc derivatives is attached through a monovalent, bivalent, or trivalent branched linker.

In one embodiment, the ligand is

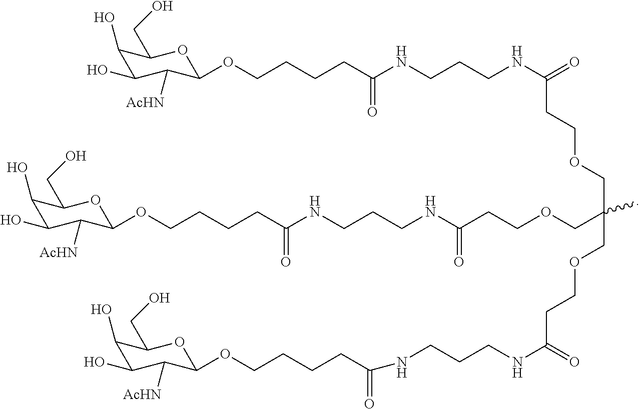 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

In one embodiment, the dsRNA agent, or salt thereof, is conjugated to the ligand as shown in the following schematic

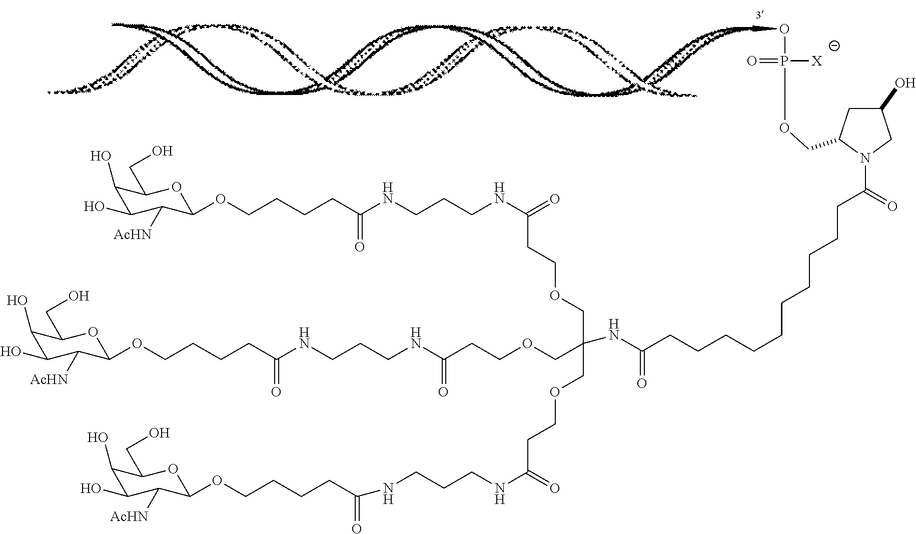

In one embodiment, the X is O.

In one embodiment, the nucleotide sequence of the sense strand differs by no more than 3 nucleotides from the nucleotide sequence 5′-gsascuuuCfaUfCfCfuggaaauaua-3′ (SEQ ID NO:35) and the nucleotide sequence of the antisense strand differs by no more than 3 nucleotides from the nucleotide sequence 5′-usAfsuauUfuCfCfaggaUfgAfaagucscsa-3′ (SEQ ID NO:36),

wherein Af is a 2′-fluoroadenosine-3′-phosphate; Afs is 2′-fluoroadenosine-3′-phosphorothioate; Cf is a 2′-fluorocytidine-3′-phosphate; U is a Uridine-3′-phosphate; Uf is a 2′-fluorouridine-3′-phosphate; a is a 2′-O-methyladenosine-3′-phosphate; as is a 2′-O-methyladenosine-3′-phosphorothioate; c is a 2′-O-methylcytidine-3′-phosphate; cs is a 2′-O-methylcytidine-3′-phosphorothioate; g is a 2′-O-methylguanosine-3′-phosphate; gs is a 2′-O-methylguanosine-3′-phosphorothioate; u is a 2′-O-methyluridine-3′-phosphate; us is a 2′-O-methyluridine-3′-phosphorothioate; and s is a phosphorothioate linkage.

In one embodiment, the nucleotide sequence of the sense strand differs by no more than 2 nucleotides from the nucleotide sequence 5′-gsascuuuCfaUfCfCfuggaaauaua-3′ (SEQ ID NO:35) and the nucleotide sequence of the antisense strand differs by no more than 2 nucleotides from the nucleotide sequence 5′-usAfsuauUfuCfCfaggaUfgAfaagucscsa-3′ (SEQ ID NO:36).

In one embodiment, the nucleotide sequence of the sense strand differs by no more than 1 nucleotide from the nucleotide sequence 5′-gsascuuuCfaUfCfCfuggaaauaua-3′ (SEQ ID NO:35) and the nucleotide sequence of the antisense strand differs by no more than 1 nucleotide from the nucleotide sequence 5′-usAfsuauUfuCfCfaggaUfgAfaagucscsa-3′ (SEQ ID NO:36).

In one embodiment, the nucleotide sequence of the sense strand comprises the nucleotide sequence 5′-gsascuuuCfaUfCfCfuggaaauaua-3′ (SEQ ID NO:35) and the nucleotide sequence of the antisense strand comprises the nucleotide sequence 5′-usAfsuauUfuCfCfaggaUfgAfaagucscsa-3′ (SEQ ID NO:36).

In one embodiment, the dsRNA agent, or salt thereof, is conjugated to a ligand as shown in the following schematic

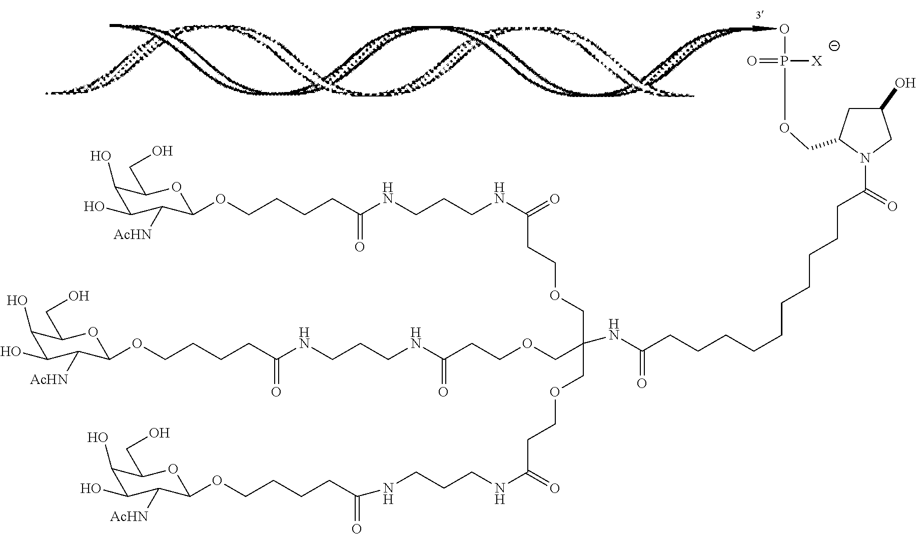

and, wherein X is O or S.

In one aspect, the present invention provides a method for inhibiting the expression of hydroxyacid oxidase (HAO1) in a subject having a non-primary hyperoxaluria disease or disorder that would benefit from reduction in urinary oxalate, comprising administering to the subject a fixed dose of about 200 mg to about 600 mg of a double stranded ribonucleic acid (dsRNA) agent, or salt thereof, which inhibits the expression of of HAO1, wherein the dsRNA agent, or salt thereof, comprises a sense strand and an antisense strand forming a double-stranded region, wherein the nucleotide sequence of the sense strand differs by no more than 3 nucleotides from the nucleotide sequence 5′-gsascuuuCfaUfCfCfuggaaauaua-3′ (SEQ ID NO:35) and the nucleotide sequence of the antisense strand differs by no more than 3 nucleotides from the nucleotide sequence 5′-usAfsuauUfuCfCfaggaUfgAfaagucscsa-3′ (SEQ ID NO:36), wherein Af is a 2′-fluoroadenosine-3′-phosphate; Afs is 2′-fluoroadenosine-3′-phosphorothioate; Cf is a 2′-fluorocytidine-3′-phosphate; U is a Uridine-3′-phosphate; Uf is a 2′-fluorouridine-3′-phosphate; a is a 2′-O-methyladenosine-3′-phosphate; as is a 2′-O-methyladenosine-3′-phosphorothioate; c is a 2′-O-methylcytidine-3′-phosphate; cs is a 2′-O-methylcytidine-3′-phosphorothioate; g is a 2′-O-methylguanosine-3′-phosphate; gs is a 2′-O-methylguanosine-3′-phosphorothioate; u is a 2′-O-methyluridine-3′-phosphate; us is a 2′-O-methyluridine-3′-phosphorothioate; and s is a phosphorothioate linkage, thereby inhibiting the expression of HAO1 in the subject.

In another aspect, the present invention provides a method for reducing urinary oxalate levels in a subject having a non-primary hyperoxaluria disease or disorder that would benefit from reduction in urinary oxalate, comprising administering to the subject a fixed dose of about 200 mg to about 600 mg of a double stranded ribonucleic acid (dsRNA) agent, or salt thereof, which inhibits the expression of of HAO1, wherein the dsRNA agent, or salt thereof, comprises a sense strand and an antisense strand forming a double-stranded region, wherein the nucleotide sequence of the sense strand differs by no more than 3 nucleotides from the nucleotide sequence 5′-gsascuuuCfaUfCfCfuggaaauaua-3′ (SEQ ID NO:35) and the nucleotide sequence of the antisense strand differs by no more than 3 nucleotides from the nucleotide sequence 5′-usAfsuauUfuCfCfaggaUfgAfaagucscsa-3′ (SEQ ID NO:36), wherein Af is a 2′-fluoroadenosine-3′-phosphate; Afs is 2′-fluoroadenosine-3′-phosphorothioate; Cf is a 2′-fluorocytidine-3′-phosphate; U is a Uridine-3′-phosphate; Uf is a 2′-fluorouridine-3′-phosphate; a is a 2′-O-methyladenosine-3′-phosphate; as is a 2′-O-methyladenosine-3′-phosphorothioate; c is a 2′-O-methylcytidine-3′-phosphate; cs is a 2′-O-methylcytidine-3′-phosphorothioate; g is a 2′-O-methylguanosine-3′-phosphate; gs is a 2′-O-methylguanosine-3′-phosphorothioate; u is a 2′-O-methyluridine-3′-phosphate; us is a 2′-O-methyluridine-3′-phosphorothioate; and s is a phosphorothioate linkage, thereby reducing urinary oxalate levels in the subject.

In one embodiment, the urinary oxalate is urinary calcium oxalate.

In one embodiment, the reduction in urinary calcium oxalate is reduction in urinary calcium oxalate supersaturation.

In one embodiment, the present invention provides a method for treating a subject having a non-primary hyperoxaluria disease or disorder that would benefit from reduction in oxalate, comprising administering to the subject a fixed dose of about 200 mg to about 600 mg of a double stranded ribonucleic acid (dsRNA) agent, or salt thereof, which inhibits the expression of of HAO1, wherein the dsRNA agent, or salt thereof, comprises a sense strand and an antisense strand forming a double-stranded region, wherein the nucleotide sequence of the sense strand differs by no more than 3 nucleotides from the nucleotide sequence 5′-gsascuuuCfaUfCfCfuggaaauaua-3′ (SEQ ID NO:35) and the nucleotide sequence of the antisense strand differs by no more than 3 nucleotides from the nucleotide sequence 5′-usAfsuauUfuCfCfaggaUfgAfaagucscsa-3′ (SEQ ID NO:36), wherein Af is a 2′-fluoroadenosine-3′-phosphate; Afs is 2′-fluoroadenosine-3′-phosphorothioate; Cf is a 2′-fluorocytidine-3′-phosphate; U is a Uridine-3′-phosphate; Uf is a 2′-fluorouridine-3′-phosphate; a is a 2′-O-methyladenosine-3′-phosphate; as is a 2′-O-methyladenosine-3′-phosphorothioate; c is a 2′-O-methylcytidine-3′-phosphate; cs is a 2′-O-methylcytidine-3′-phosphorothioate; g is a 2′-O-methylguanosine-3′-phosphate; gs is a 2′-O-methylguanosine-3′-phosphorothioate; u is a 2′-O-methyluridine-3′-phosphate; us is a 2′-O-methyluridine-3′-phosphorothioate; and s is a phosphorothioate linkage, thereby treating the subject having the non-primary hyperoxaluria disease or disorder that would benefit from reduction in oxalate.

In one embodiment, the non-primary hyperoxaluria disease or disorder is selected from the group consisting of secondary hyperoxaluria, a kidney stone disease, chronic kidney disease (CKD), end-stage renal disease (ESRD), coronary artery disease, cutaneous oxalate deposition, ethylene glycol poisoning, planned kidney transplantation, and previous kidney transplantation.

In one embodiment, the non-primary hyperoxaluria disease or disorder is a kidney stone disease.

In one embodiment, the kidney stone disease is calcium oxalate kidney stone disease.

In one embodiment, the calcium oxalate kidney stone disease is recurrent calcium oxalate kidney stone disease.

In one embodiment, administration of the dsRNA agent, or salt thereof, to the subject reduces urinary oxalate levels.

In one embodiment, the urinary oxalate is urinary calcium oxalate.

In one embodiment, the reduction in urinary calcium oxalate is reduction in urinary calcium oxalate supersaturation.

In one embodiment, administration of the dsRNA agent, or salt thereof, to the subject reduces clinical and radiographic kidney stone events.

In one embodiment, the subject is a human.

In one embodiment, the dsRNA agent, or salt thereof, is administered to the subject at an interval of once every six months.

In one embodiment, the dsRNA agent, or salt thereof, is administered to the subject initially, at three months, and every six months thereafter.

In one embodiment, the fixed dose of the dsRNA agent, or salt thereof, is about 284 mg.

In one embodiment, the fixed dose of the dsRNA agent, or salt thereof, is about 567 mg.

In one embodiment, the dsRNA agent, or salt thereof, is administered to the subject subcutaneously.

In one embodiment, the subcutaneous administration is subcutaneous injection.

In one embodiment, the nucleotide sequence of the sense strand differs by no more than 2 nucleotides from the nucleotide sequence 5′-gsascuuuCfaUfCfCfuggaaauaua-3′ (SEQ ID NO:35) and the nucleotide sequence of the antisense strand differs by no more than 2 nucleotides from the nucleotide sequence 5′-usAfsuauUfuCfCfaggaUfgAfaagucscsa-3′ (SEQ ID NO:36).

In one embodiment, the nucleotide sequence of the sense strand differs by no more than 1 nucleotide from the nucleotide sequence 5′-gsascuuuCfaUfCfCfuggaaauaua-3′ (SEQ ID NO:35) and the nucleotide sequence of the antisense strand differs by no more than 1 nucleotide from the nucleotide sequence 5′-usAfsuauUfuCfCfaggaUfgAfaagucscsa-3′ (SEQ ID NO:36).

In one embodiment, the nucleotide sequence of the sense strand comprises the nucleotide sequence 5′-gsascuuuCfaUfCfCfuggaaauaua-3′ (SEQ ID NO:35) and the nucleotide sequence of the antisense strand comprises the nucleotide sequence 5′-usAfsuauUfuCfCfaggaUfgAfaagucscsa-3′ (SEQ ID NO:36).

In one embodiment, the dsRNA agent, or salt thereof, is conjugated to a ligand as shown in the following schematic

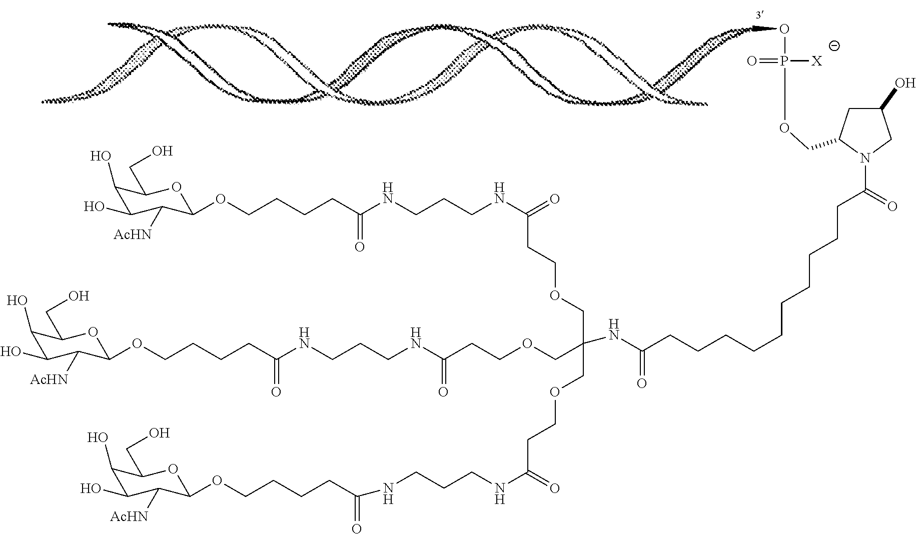

and, wherein X is O or S.

In one embodiment, the X is O.

In one embodiment, the dsRNA agent is in salt form.

In one embodiment, the dsRNA agent, or salt thereof, is administered to the subject in a pharmaceutical formulation.

In one embodiment, the method of the invention further comprise administering an additional therapeutic to the subject.

In one aspect, the present invention provides a method for reducing calcium oxalate kidney stone incidence in a subject, the method comprising subcutaneously administering to the subject a fixed dose of about 284 mg or about 567 mg of a double stranded ribonucleic acid (dsRNA) agent, or salt thereof, comprising a sense strand and an antisense strand forming a double-stranded region, wherein the sense strand comprises the nucleotide sequence 5′-gsascuuuCfaUfCfCfuggaaauaua-3′ (SEQ ID NO:35) and the antisense strand comprises the nucleotide sequence 5′-usAfsuauUfuCfCfaggaUfgAfaagucscsa-3′ (SEQ ID NO:36), wherein Af is a 2′-fluoroadenosine-3′-phosphate; Afs is 2′-fluoroadenosine-3′-phosphorothioate; Cf is a 2′-fluorocytidine-3′-phosphate; U is a Uridine-3′-phosphate; Uf is a 2′-fluorouridine-3′-phosphate; a is a 2′-O-methyladenosine-3′-phosphate; as is a 2′-O-methyladenosine-3′-phosphorothioate; c is a 2′-O-methylcytidine-3′-phosphate; cs is a 2′-O-methylcytidine-3′-phosphorothioate; g is a 2′-O-methylguanosine-3′-phosphate; gs is a 2′-O-methylguanosine-3′-phosphorothioate; u is a 2′-O-methyluridine-3′-phosphate; us is a 2′-O-methyluridine-3′-phosphorothioate; and s is a phosphorothioate linkage, and wherein the sense strand is conjugated to a ligand as shown in the following schematic

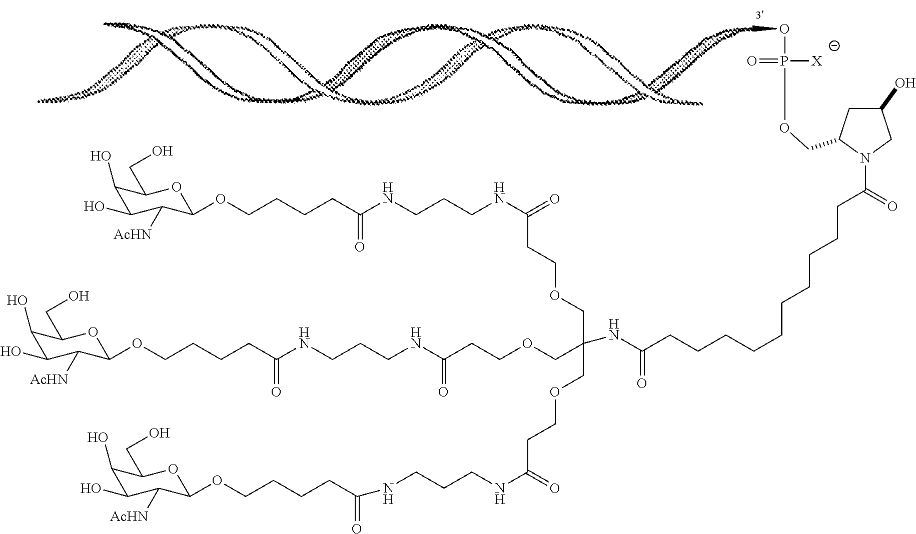

and, wherein X is O, thereby reducing calcium oxalate kidney stone incidence in the subject.

In one embodiment, the subject has suffered 2 or more oxalate stone events.

In one embodiment, the subject has elevated urinary oxalate levels.

In one embodiment, the subject has suffered 2 or more oxalate stone events and has elevated urinary oxalate levels.

In one embodiment, the dsRNA agent, or salt thereof, is administered to the subject once every six months.

In one embodiment, the dsRNA agent, or salt thereof, is administered to the subject initially, at three months, and every six months thereafter.

In one aspect, the present invention provides a method for treating a subject having a non-primary hyperoxaluria disease or disorder that would benefit from reduction in oxalate, the method comprising administering to the subject a therapeutically effective amount of a nucleic acid inhibitor of hydroxyacid oxidase (HAO1) and/or a nucleic acid inhibitor of Proline Dehydrogenase 2 (PRODH2), thereby treating the subject having the non-primary hyperoxaluria disease or disorder that would benefit from reduction in oxalate.

In some embodiments, the non-primary hyperoxaluria disease or disorder is selected from the group consisting of a secondary hyperoxaluria, a kidney stone disease, chronic kidney disease (CKD), end-stage renal disease (ESRD), coronary artery disease, cutaneous oxalate deposition, ethylene glycol poisoning, planned kidney transplantation, and previous kidney transplantation.

In another aspect, the present invention provides a method of treating a subject at risk of developing a non-primary hyperoxaluria disease or disorder that would benefit from reduction in oxalate, the method comprising administering to the subject a therapeutically effective amount of a nucleic acid inhibitor of lactate dehydrogenase A (LDHA), a nucleic acid inhibitor of hydroxyacid oxidase (HAO1), and/or a nucleic acid inhibitor of Proline Dehydrogenase 2 (PRODH2), thereby treating the subject at risk of developing the non-primary hyperoxaluria disease or disorder that would benefit from reduction in oxalate.

In some embodiments, subject at risk of developing a non-primary hyperoxaluria disease or disorder that would benefit from reduction in oxalate suffers from Crohn's disease, inflammatory bowel disease, a bariatric surgery, fibromyalgia, an autoimmune disease, coronary artery disease, a kidney stone disease, end-stage renal disease (ESRD), diabetes, obesity, HIV, or ethylene glycol poisoning.

In one embodiment, the subject is a human.

In one embodiment, the nucleic acid inhibitor is a double stranded ribonucleic acid (dsRNA) agent that inhibits the expression of HAO1.

In one embodiment, the dsRNA agent comprises a sense strand and an antisense strand forming a double stranded region, wherein the sense strand comprises a nucleotide sequence comprising at least 15 contiguous nucleotides differing by no more than 3 nucleotides from a portion of the nucleotide sequence of SEQ ID NO: 21 and the antisense strand comprises a nucleotide sequence comprising at least 15 contiguous nucleotides differing by no more than 3 nucleotides from the corresponding portion of nucleotide sequence of SEQ ID NO: 22 such that the sense strand is complementary to the at least 15 contiguous nucleotides in the antisense strand.

In one embodiment, the dsRNA agent comprises a sense strand and an antisense strand forming a double stranded region, wherein the antisense strand comprises at least 15 contiguous nucleotides differing by no more than 3 nucleotides from any one of the antisense sequences listed in any one of Tables 4-14.

In one embodiment, the dsRNA agent comprises a sense strand and an antisense strand forming a double-stranded region, wherein the sense strand comprises the nucleotide sequence 5′-GACUUUCAUCCUGGAAAUAUA-3′ (SEQ ID NO:33) and the antisense strand comprises the nucleotide sequence 5′-UAUAUUUCCAGGAUGAAAGUCCA-3′ (SEQ ID NO:34).

In one embodiment, the nucleic acid inhibitor is a double stranded ribonucleic acid (dsRNA) agent that inhibits the expression of LDHA.

In one embodiment, the dsRNA agent comprises a sense strand and an antisense strand forming a double stranded region, wherein the sense strand comprises a nucleotide sequence comprising at least 15 contiguous nucleotides differing by no more than 3 nucleotides from a portion of the nucleotide sequence of SEQ ID NO: 1 and the antisense strand comprises a nucleotide sequence comprising at least 15 contiguous nucleotides differing by no more than 3 nucleotides from the corresponding portion of nucleotide sequence of SEQ ID NO: 2 such that the sense strand is complementary to the at least 15 contiguous nucleotides in the antisense strand.

In one embodiment, the dsRNA agent comprises a sense strand and an antisense strand forming a double stranded region, wherein the antisense strand comprises at least 15 contiguous nucleotides differing by no more than 3 nucleotides from any one of the antisense sequences listed in any one of Tables 2-3.

In one embodiment, the dsRNA agent comprises a sense strand and an antisense strand forming a double stranded region, wherein the sense strand comprises a nucleotide sequence comprising at least 15 contiguous nucleotides differing by no more than 3 nucleotides from the nucleotide sequence of 5′-AUGUUGUCCUUUUUAUCUGAGCAGCCGAAAGGCUGC-3′ (SEQ ID NO:31), and the antisense strand comprises a nucleotide sequence comprising at least 15 contiguous nucleotides differing by no more than 3 nucleotides from the nucleotide sequence 5′-UCAGAUAAAAAGGACAACAUGG-3′ (SEQ ID NO: 32).

one embodiment, the nucleic acid inhibitor is a double stranded ribonucleic acid (dsRNA) agent that inhibits the expression of PRODH2.

In one embodiment, the dsRNA agent comprises a sense strand and an antisense strand forming a double stranded region, wherein the sense strand comprises a nucleotide sequence comprising at least 15 contiguous nucleotides differing by no more than 3 nucleotides from a portion of the nucleotide sequence of SEQ ID NO: 4641 and the antisense strand comprises a nucleotide sequence comprising at least 15 contiguous nucleotides differing by no more than 3 nucleotides from the corresponding portion of nucleotide sequence of SEQ ID NO: 4642 such that the sense strand is complementary to the at least 15 contiguous nucleotides in the antisense strand.

In one embodiment, the dsRNA agent comprises a sense strand and an antisense strand forming a double stranded region, wherein the antisense strand comprises at least 15 contiguous nucleotides differing by no more than 3 nucleotides from any one of the antisense sequences listed in any one of Tables 15-16.

In one embodiment, the nucleic acid inhibitor is a dual targeting double stranded ribonucleic acid (dsRNA) agent that inhibits the expression of LDHA and HAO1.

In one embodiment, the dual targeting dsRNA agent comprises a first double stranded ribonucleic acid (dsRNA) agent that inhibits expression of lactic dehydrogenase A (LDHA) comprising a sense strand and an antisense strand; and a second double stranded ribonucleic acid (dsRNA) agent that inhibits expression of hydroxyacid oxidase 1 (glycolate oxidase) (HAO1) comprising a sense strand and an antisense strand, wherein the first dsRNA agent and the second dsRNA agent are covalently attached, wherein the sense strand of the first dsRNA agent comprises at least 15 contiguous nucleotides differing by no more than 3 nucleotides from the nucleotide sequence of SEQ ID NO: 1, and the antisense strand of the first dsRNA agent comprises at least 15 contiguous nucleotides differing by no more than 3 nucleotides from the nucleotide sequence of SEQ ID NO:2, wherein the sense strand of the second dsRNA agent comprises at least 15 contiguous nucleotides differing by no more than 3 nucleotides from the nucleotide sequence of SEQ ID NO:21, and said antisense strand of the second dsRNA agent comprises at least 15 contiguous nucleotides differing by no more than 3 nucleotides from the nucleotide sequence of SEQ ID NO:22.

In one embodiment, the dual targeting dsRNA agent comprises a first double stranded ribonucleic acid (dsRNA) agent that inhibits expression of lactic dehydrogenase A (LDHA) comprising a sense strand and an antisense strand; and a second double stranded ribonucleic acid (dsRNA) agent that inhibits expression of hydroxyacid oxidase 1 (glycolate oxidase) (HAO1) comprising a sense strand and an antisense strand, wherein the first dsRNA agent and the second dsRNA agent are covalently attached, wherein the antisense strand of the first dsRNA agent comprises at least 15 contiguous nucleotides differing by no more than 3 nucleotides from any one of the antisense sequences listed in any one of Tables 2-3, and wherein the antisense strand of the second dsRNA agent comprises at least 15 contiguous nucleotides differing by no more than 3 nucleotides from any one of the antisense sequences listed in any one of Tables 4-14.

In one embodiment, the dsRNA agent comprises at least one modified nucleotide.

In one embodiment, no more than five of the sense strand nucleotides and no more than five of the nucleotides of the antisense strand are unmodified nucleotides.

In one embodiment, all of the nucleotides of the sense strand and all of the nucleotides of the antisense strand are modified nucleotides.

In one embodiment, at least one of the modified nucleotides is selected from the group a deoxy-nucleotide, a 3′-terminal deoxythimidine (dT) nucleotide, a 2′-O-methyl modified nucleotide, a 2′-fluoro modified nucleotide, a 2′-deoxy-modified nucleotide, a locked nucleotide, an unlocked nucleotide, a conformationally restricted nucleotide, a constrained ethyl nucleotide, an abasic nucleotide, a 2′-amino-modified nucleotide, a 2′-O-allyl-modified nucleotide, 2′-C-alkyl-modified nucleotide, 2′-hydroxyl-modified nucleotide, a 2′-methoxyethyl modified nucleotide, a 2′-O-alkyl-modified nucleotide, a morpholino nucleotide, a phosphoramidate, a non-natural base comprising nucleotide, a tetrahydropyran modified nucleotide, a 1,5-anhydrohexitol modified nucleotide, a cyclohexenyl modified nucleotide, a nucleotide comprising a 5′-phosphorothioate group, a nucleotide comprising a 5′-methylphosphonate group, a nucleotide comprising a 5′ phosphate or 5′ phosphate mimic, a nucleotide comprising vinyl phosphonate, a nucleotide comprising adenosine-glycol nucleic acid (GNA), a nucleotide comprising thymidine-glycol nucleic acid (GNA) S-Isomer, a nucleotide comprising 2-hydroxymethyl-tetrahydrofurane-5-phosphate, a nucleotide comprising 2′-deoxythymidine-3′phosphate, a nucleotide comprising 2′-deoxyguanosine-3′-phosphate, and a terminal nucleotide linked to a cholesteryl derivative and a dodecanoic acid bisdecylamide group; and combinations thereof.

In one embodiment, the dsRNA agent further comprises at least one phosphorothioate internucleotide linkage.

In one embodiment, the dsRNA agent comprises 6-8 phosphorothioate internucleotide linkages.

In one embodiment, at least one strand of the dsRNA agent further comprises a ligand.

In one embodiment, the ligand is attached to the 3′ end of the sense strand.

In one embodiment, the ligand is one or more N-acetylgalactosamine (GalNAc) derivatives.

In one embodiment, the one or more GalNAc derivatives is attached through a monovalent, bivalent, or trivalent branched linker.

In one embodiment, the ligand is

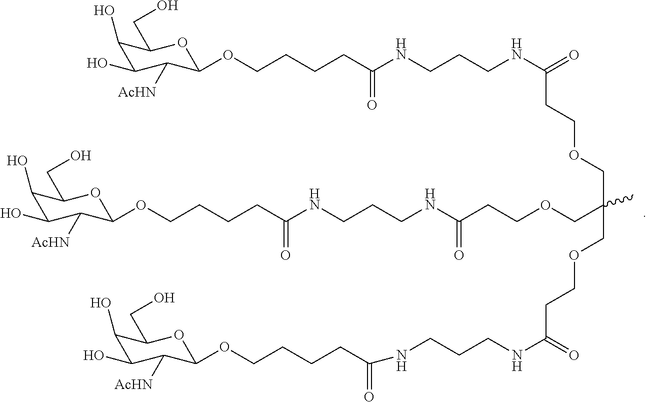 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

In one embodiment, the dsRNA agent is conjugated to the ligand as shown in the following schematic

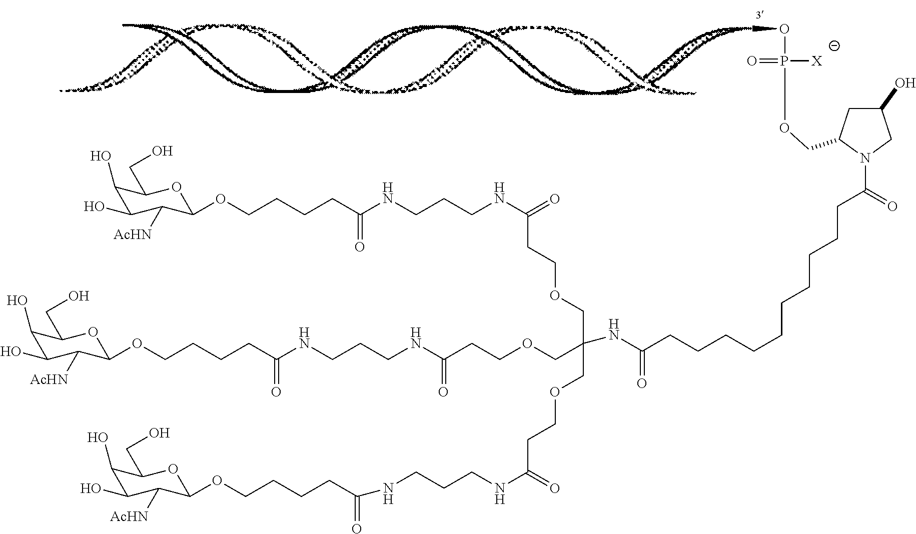

and, wherein X is O or S.

In one embodiment, the X is O.

In one embodiment, the sense strand comprises the nucleotide sequence 5′-gsascuuuCfaUfCfCfuggaaauaua-3′ (SEQ ID NO:35) and the antisense strand comprises the nucleotide sequence 5′-usAfsuauUfuCfCfaggaUfgAfaagucscsa-3′ (SEQ ID NO:36), wherein Af is a 2′-fluoroadenosine-3′-phosphate; Afs is 2′-fluoroadenosine-3′-phosphorothioate; Cf is a 2′-fluorocytidine-3′-phosphate; U is a Uridine-3′-phosphate; Uf is a 2′-fluorouridine-3′-phosphate; a is a 2′-O-methyladenosine-3′-phosphate; as is a 2′-O-methyladenosine-3′-phosphorothioate; c is a 2′-O-methylcytidine-3′-phosphate; cs is a 2′-O-methylcytidine-3′-phosphorothioate; g is a 2′-O-methylguanosine-3′-phosphate; gs is a 2′-O-methylguanosine-3′-phosphorothioate; u is a 2′-O-methyluridine-3′-phosphate; us is a 2′-O-methyluridine-3′-phosphorothioate; and s is a phosphorothioate linkage.

In one embodiment, the dsRNA agent is conjugated to the ligand as shown in the following schematic

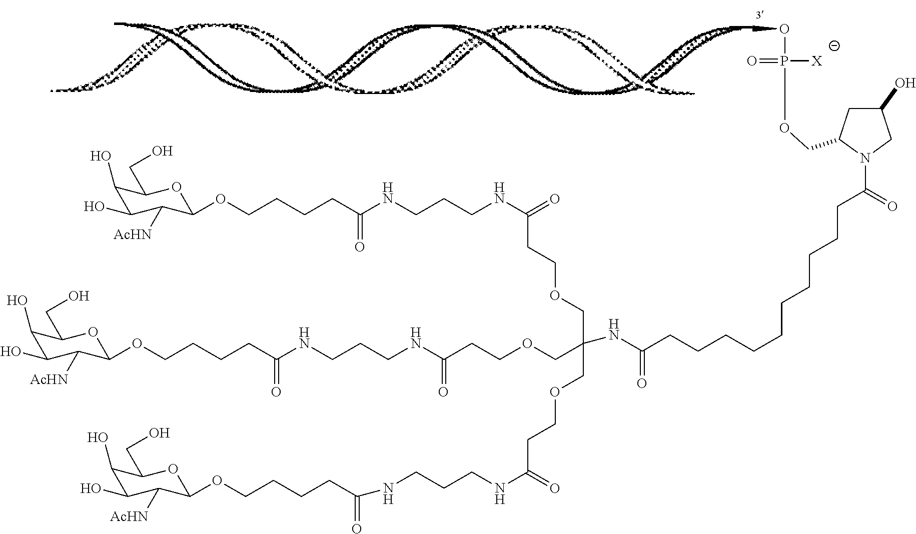

and, wherein X is O or S.

In one embodiment, the dsRNA agent comprises at least one modified nucleotide.

In one embodiment, all of the nucleotides of the dsRNA agent are modified nucleotides.

In one embodiment, the modified nucleotide comprises a 2′-modification.

In one embodiment, the 2′-modification is a 2′-fluoro or 2′-O-methyl modification.

In one embodiment, one or more of the following positions are modified with a 2′-O-methyl: positions 1, 2, 4, 6, 7, 12, 14, 16, 18-26, or 31-36 of the sense strand and/or positions 1, 6, 8, 11-13, 15, 17, or 19-22 of the antisense strand.

In one embodiment, all of positions 1, 2, 4, 6, 7, 12, 14, 16, 18-26, and 31-36 of the sense strand and all of the positions 1, 6, 8, 11-13, 15, 17, and 19-22 of the antisense strand are modified with a 2-O-methyl.

In one embodiment, one or more of the following positions are modified with a 2′-fluoro: positions 3, 5, 8-11, 13, 15, or 17 of the sense strand and/or positions 2-5, 7, 9, 10, 14, 16, or 18 of the antisense strand.

In one embodiment, all of positions 3, 5, 8-11, 13, 15, or 17 of the sense strand and all of positions 2-5, 7, 9, 10, 14, 16, and 18 of the antisense strand are modified with a 2′-fluoro.

In one embodiment, the dsRNA agent comprises at least one modified internucleotide linkage.

In one embodiment, the at least one modified internucleotide linkage is a phosphorothioate linkage.

In one embodiment, the dsRNA agent has a phosphorothioate linkage between one or more of: positions 1 and 2 of the sense strand, positions 1 and 2 of the antisense strand, positions 2 and 3 of the antisense strand, positions 3 and 4 of the antisense strand, positions 20 and 21 of the antisense strand, and positions 21 and 22 of the antisense strand.

In one embodiment, the dsRNA agent has a phosphorothioate linkage between each of: positions 1 and 2 of the sense strand, positions 1 and 2 of the antisense strand, positions 2 and 3 of the antisense strand, positions 3 and 4 of the antisense strand, positions 20 and 21 of the antisense strand, and positions 21 and 22 of the antisense strand.

In one embodiment, the uridine at the first position of the antisense strand comprises a phosphate analog.

In one embodiment, the dsRNA comprises the following structure at position 1 of the antisense strand:

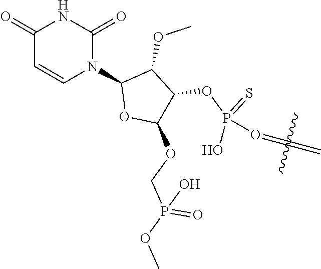

In one embodiment, one or more of the nucleotides of the -GAAA- sequence on the sense strand is conjugated to a monovalent GalNac moiety.

In one embodiment, each of the nucleotides of the -GAAA- sequence on the sense strand is conjugated to a monovalent GalNac moiety.

In one embodiment, the -GAAA- motif comprises the structure:

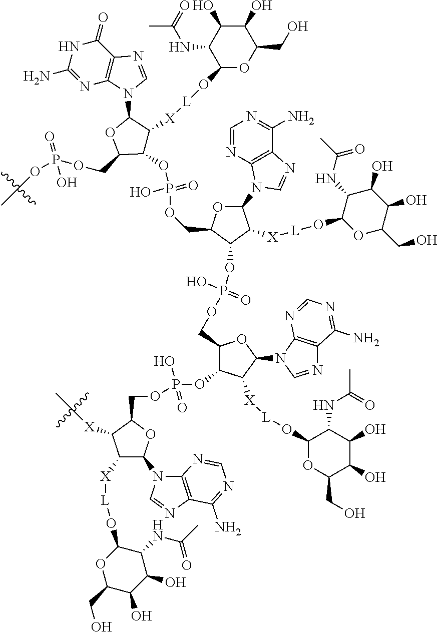

wherein: L represents a bond, click chemistry handle, or a linker of 1 to 20, inclusive, consecutive, covalently bonded atoms in length, selected from the group consisting of substituted and unsubstituted alkylene, substituted and unsubstituted alkenylene, substituted and unsubstituted alkynylene, substituted and unsubstituted heteroalkylene, substituted and unsubstituted heteroalkenylene, substituted and unsubstituted heteroalkynylene, and combinations thereof; and

### X is a O, S, or N.

In one embodiment, L is an acetal linker.

In one embodiment, X is O.

In one embodiment, the -G AAA- sequence comprises the structure:

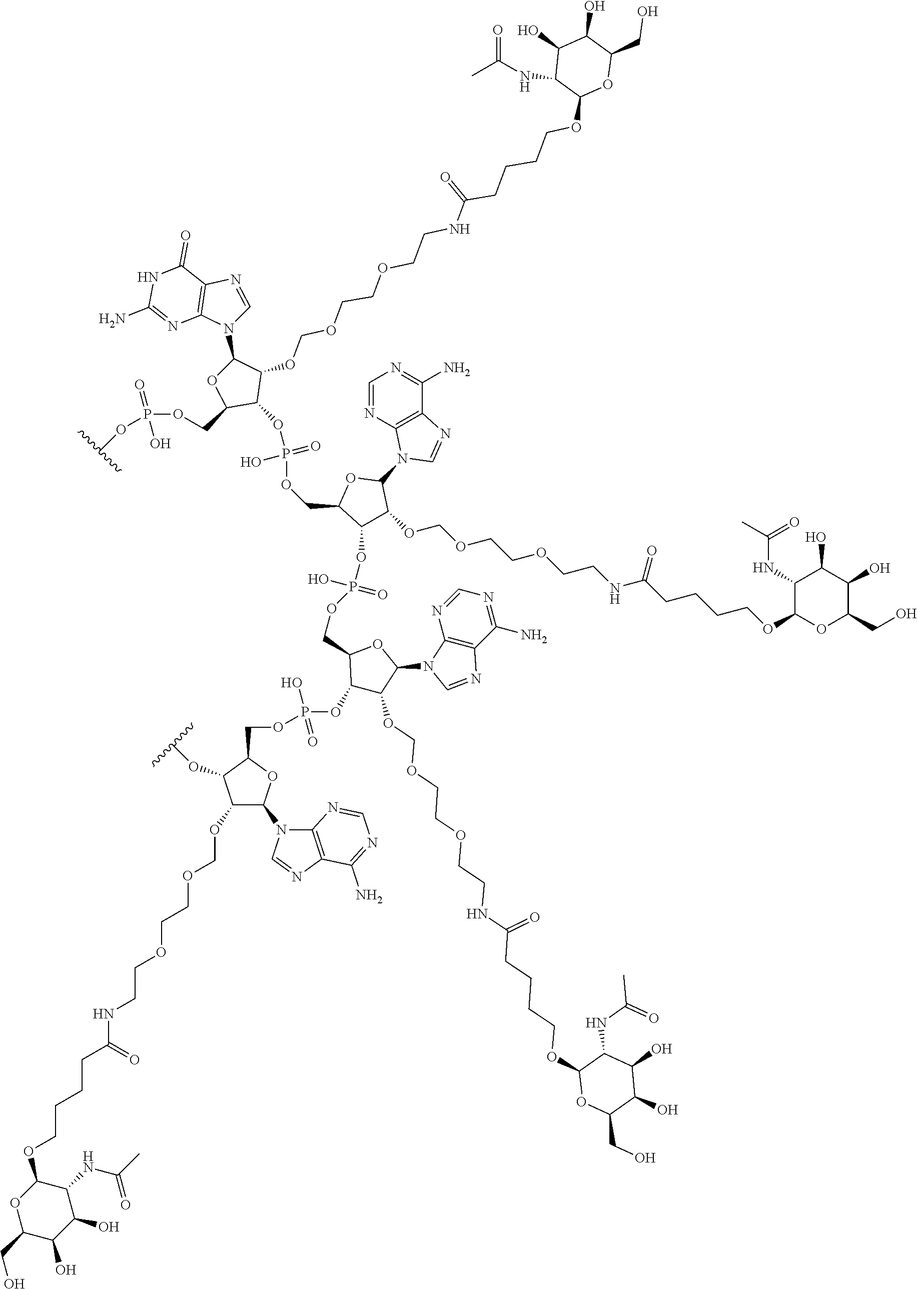

In one embodiment, the dsRNA comprises an antisense strand having a sequence set forth as UCAGAUAAAAAGGACAACAUGG (SEQ ID NO: 32) and a sense strand having a sequence set forth as AUGUUGUCCUUUUUAUCUGAGCAGCCGAAAGGCUGC (SEQ ID NO: 31), wherein all of positions 1, 2, 4, 6, 7, 12, 14, 16, 18-26, and 31-36 of the sense strand and all of positions 1, 6, 8, 11-13, 15, 17, and 19-22 of the antisense strand are modified with a 2′-O-methyl, and all of positions 3, 5, 8-11, 13, 15, or 17 of the sense strand and all of positions 2-5, 7, 9, 10, 14, 16, and 18 of the antisense strand are modified with a 2′-fluoro; wherein the oligonucleotide has a phosphorothioate linkage between each of: positions 1 and 2 of the sense strand, positions 1 and 2 of the antisense strand, positions 2 and 3 of the antisense strand, positions 3 and 4 of the antisense strand, positions 20 and 21 of the antisense strand, and positions 21 and 22 of the antisense strand;

wherein the dsRNA agent comprises the following structure at position 1 of the antisense strand:

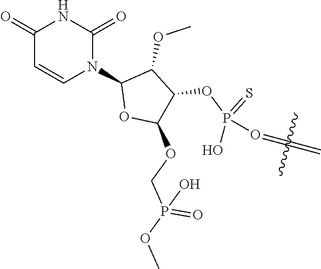

wherein each of the nucleotides of the -GAAA- sequence on the sense strand is conjugated to a monovalent GalNac moiety comprising the structure:

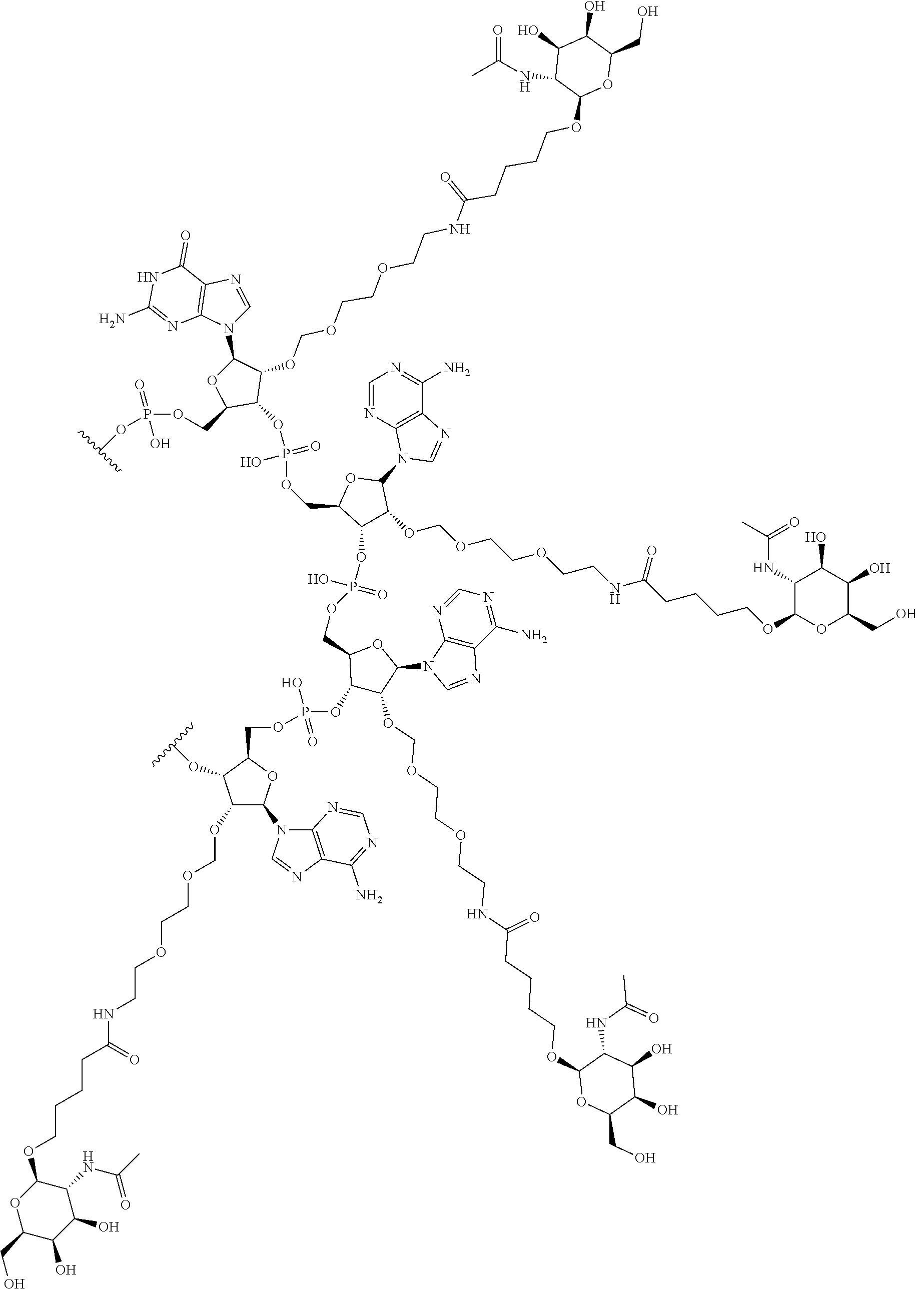

In one embodiment, the dsRNA agent is present in a composition comprising the dsRNA agent and Na+ counterions.

In one embodiment, the nucleic acid inhibitor is a single stranded antisense polynucleotide agent that inhibits the expression of LDHA.

In one embodiment, the single stranded antisense polynucleotide agent comprises at least 15 contiguous nucleotide differing by no more than 3 nucleotides from any one of the antisense nucleotide sequences in any one of Tables 2-3.

In one embodiment, the nucleic acid inhibitor is a single stranded antisense polynucleotide agent that inhibits the expression of PRODH2.

In one embodiment, the single stranded antisense polynucleotide agent comprises at least 15 contiguous nucleotide differing by no more than 3 nucleotides from any one of the antisense nucleotide sequences in any one of Tables 15-16.

In one embodiment, the single stranded antisense polynucleotide agent is about 8 to about 50 nucleotides in length.

In one embodiment, substantially all of the nucleotides of the single stranded antisense polynucleotide agent are modified nucleotides.

In one embodiment, all of the nucleotides of the single stranded antisense polynucleotide agent are modified nucleotides.

In one embodiment, the modified nucleotide comprises a modified sugar moiety selected from the group consisting of: a 2′-O-methoxyethyl modified sugar moiety, a 2′-O-alkyl modified sugar moiety, and a bicyclic sugar moiety.

In one embodiment, the bicyclic sugar moiety has a (—CRH—)n group forming a bridge between the 2′ oxygen and the 4′ carbon atoms of the sugar ring, wherein n is 1 or 2 and wherein R is H, CH₃ or CH₃OCH₃.

In one embodiment, n is 1 and R is CH₃.

In one embodiment, the modified nucleotide is a 5-methylcytosine.

In one embodiment, the single stranded antisense polynucleotide agent comprises a modified internucleoside linkage.

In one embodiment, the modified internucleoside linkage is a phosphorothioate internucleoside linkage.

In one embodiment, the single stranded antisense polynucleotide agent comprises a plurality of 2′-deoxynucleotides flanked on each side by at least one nucleotide having a modified sugar moiety.

In one embodiment, the single stranded antisense polynucleotide agent is a gapmer comprising a gap segment comprised of linked 2′-deoxynucleotides positioned between a 5′ and a 3′ wing segment.

In one embodiment, the modified sugar moiety is selected from the group consisting of a 2′-O-methoxyethyl modified sugar moiety, a 2′-methoxy modified sugar moiety, a 2′-O-alkyl modified sugar moiety, and a bicyclic sugar moiety.

In one embodiment, the nucleic acid inhibitor is present in a pharmaceutical formulation.

In some embodiments, the methods of the invention further comprise administering an additional therapeutic to the subject.

In one embodiment, the nucleic acid inhibitor is administered to the subject at a dose of about 0.01 mg/kg to about 10 mg/kg or about 0.5 mg/kg to about 50 mg/kg.

In one embodiment, the nucleic acid inhibitor is administered to the subject subcutaneously.

The present invention also provides methods for treating a subject having chronic kidney disease (CKD). The methods include administering to the subject a weight-based dose of a dsRNA agent, or salt thereof, which inhibits the expression of HAO1 in a doing regimen which includes a loading phase of closely spaced administrations that may be followed by a maintenance phase, in which the the dsRNA agent, or salt thereof, is administered at longer spaced intervals.

Accordingly, in one aspect, the present invention provides a method for inhibiting the expression of hydroxyacid oxidase (HAO1) in a subject having chronic kidney disease (CKD), comprising administering to the subject a double stranded ribonucleic acid (dsRNA) agent, or salt thereof, which inhibits the expression of of HAO1 in a dosing regimen that includes a loading phase followed by a maintenance phase, wherein the subject has a body weight of less than about 10 kilograms (kg) and the loading phase comprises administering a dose of about 6 milligram per kilogram (mg/kg) of the double stranded RNAi agent, or salt thereof, to the subject about once a month for about three months, and the maintenance phase comprises administering a dose of about 3 mg/kg of the double stranded RNAi agent, or salt thereof, to the subject about once a month; or wherein the subject has a body weight of between about 10 kg to about less than 20 kg and the loading phase comprises administering a dose of about 6 mg/kg of the double stranded RNAi agent, or salt thereof, to the subject about once a month for about three months, and the maintenance phase comprises administering a dose of about 6 mg/kg of the double stranded RNAi agent, or salt thereof, to the subject about once every three months; or wherein the subject has a body weight of greater than about 20 kg and the loading phase comprises administering a dose of about 3 mg/kg of the double stranded RNAi agent, or salt thereof, to the subject about once a month for about three months, and the maintenance phase comprises administering a dose of about 3 mg/kg of the double stranded RNAi agent, or salt thereof, to the subject about once every three months, wherein the dsRNA agent, or salt thereof, comprises a sense strand and an antisense strand forming a double-stranded region, wherein the nucleotide sequence of the sense strand differs by no more than 3 nucleotides from the nucleotide sequence 5′-gsascuuuCfaUfCfCfuggaaauaua-3′ (SEQ ID NO:35) and the nucleotide sequence of the antisense strand differs by no more than 3 nucleotides from the nucleotide sequence 5′-usAfsuauUfuCfCfaggaUfgAfaagucscsa-3′ (SEQ ID NO:36), wherein Af is a 2′-fluoroadenosine-3′-phosphate; Afs is 2′-fluoroadenosine-3′-phosphorothioate; Cf is a 2′-fluorocytidine-3′-phosphate; U is a Uridine-3′-phosphate; Uf is a 2′-fluorouridine-3′-phosphate; a is a 2′-O-methyladenosine-3′-phosphate; as is a 2′-O-methyladenosine-3′-phosphorothioate; c is a 2′-O-methylcytidine-3′-phosphate; cs is a 2′-O-methylcytidine-3′-phosphorothioate; g is a 2′-O-methylguanosine-3′-phosphate; gs is a 2′-O-methylguanosine-3′-phosphorothioate; u is a 2′-O-methyluridine-3′-phosphate; us is a 2′-O-methyluridine-3′-phosphorothioate; and s is a phosphorothioate linkage, thereby inhibiting the expression of HAO1 in the subject.

In another aspect, the present invention provides a method for reducing urinary oxalate levels in a subject having chronic kidney disease, comprising administering to the subject a double stranded ribonucleic acid (dsRNA) agent, or salt thereof, which inhibits the expression of of HAO1 in a dosing regimen that includes a loading phase followed by a maintenance phase, wherein the subject has a body weight of less than about 10 kilograms (kg) and the loading phase comprises administering a dose of about 6 milligram per kilogram (mg/kg) of the double stranded RNAi agent, or salt thereof, to the subject about once a month for about three months, and the maintenance phase comprises administering a dose of about 3 mg/kg of the double stranded RNAi agent, or salt thereof, to the subject about once a month; or wherein the subject has a body weight of between about 10 kg to about less than 20 kg and the loading phase comprises administering a dose of about 6 mg/kg of the double stranded RNAi agent, or salt thereof, to the subject about once a month for about three months, and the maintenance phase comprises administering a dose of about 6 mg/kg of the double stranded RNAi agent, or salt thereof, to the subject about once every three months; or wherein the subject has a body weight of greater than about 20 kg and the loading phase comprises administering a dose of about 3 mg/kg of the double stranded RNAi agent, or salt thereof, to the subject about once a month for about three months, and the maintenance phase comprises administering a dose of about 3 mg/kg of the double stranded RNAi agent, or salt thereof, to the subject about once every three months, wherein the dsRNA agent, or salt thereof, comprises a sense strand and an antisense strand forming a double-stranded region, wherein the nucleotide sequence of the sense strand differs by no more than 3 nucleotides from the nucleotide sequence 5′-gsascuuuCfaUfCfCfuggaaauaua-3′ (SEQ ID NO:35) and the nucleotide sequence of the antisense strand differs by no more than 3 nucleotides from the nucleotide sequence 5′-usAfsuauUfuCfCfaggaUfgAfaagucscsa-3′ (SEQ ID NO:36), wherein Af is a 2′-fluoroadenosine-3′-phosphate; Afs is 2′-fluoroadenosine-3′-phosphorothioate; Cf is a 2′-fluorocytidine-3′-phosphate; U is a Uridine-3′-phosphate; Uf is a 2′-fluorouridine-3′-phosphate; a is a 2′-O-methyladenosine-3′-phosphate; as is a 2′-O-methyladenosine-3′-phosphorothioate; c is a 2′-O-methylcytidine-3′-phosphate; cs is a 2′-O-methylcytidine-3′-phosphorothioate; g is a 2′-O-methylguanosine-3′-phosphate; gs is a 2′-O-methylguanosine-3′-phosphorothioate; u is a 2′-O-methyluridine-3′-phosphate; us is a 2′-O-methyluridine-3′-phosphorothioate; and s is a phosphorothioate linkage, thereby reducing urinary oxalate levels in the subject.

In one aspect, the present invention provides a method for treating a subject having chronic kidney disease, comprising administering to the subject a double stranded ribonucleic acid (dsRNA) agent, or salt thereof, which inhibits the expression of of HAO1 in a dosing regimen that includes a loading phase followed by a maintenance phase, wherein the subject has a body weight of less than about 10 kilograms (kg) and the loading phase comprises administering a dose of about 6 milligram per kilogram (mg/kg) of the double stranded RNAi agent, or salt thereof, to the subject about once a month for about three months, and the maintenance phase comprises administering a dose of about 3 mg/kg of the double stranded RNAi agent, or salt thereof, to the subject about once a month; or wherein the subject has a body weight of between about 10 kg to about less than 20 kg and the loading phase comprises administering a dose of about 6 mg/kg of the double stranded RNAi agent, or salt thereof, to the subject about once a month for about three months, and the maintenance phase comprises administering a dose of about 6 mg/kg of the double stranded RNAi agent, or salt thereof, to the subject about once every three months; or wherein the subject has a body weight of greater than about 20 kg and the loading phase comprises administering a dose of about 3 mg/kg of the double stranded RNAi agent, or salt thereof, to the subject about once a month for about three months, and the maintenance phase comprises administering a dose of about 3 mg/kg of the double stranded RNAi agent, or salt thereof, to the subject about once every three months, wherein the dsRNA agent, or salt thereof, comprises a sense strand and an antisense strand forming a double-stranded region, wherein the nucleotide sequence of the sense strand differs by no more than 3 nucleotides from the nucleotide sequence 5′-gsascuuuCfaUfCfCfuggaaauaua-3′ (SEQ ID NO:35) and the nucleotide sequence of the antisense strand differs by no more than 3 nucleotides from the nucleotide sequence 5′-usAfsuauUfuCfCfaggaUfgAfaagucscsa-3′ (SEQ ID NO:36), wherein Af is a 2′-fluoroadenosine-3′-phosphate; Afs is 2′-fluoroadenosine-3′-phosphorothioate; Cf is a 2′-fluorocytidine-3′-phosphate; U is a Uridine-3′-phosphate; Uf is a 2′-fluorouridine-3′-phosphate; a is a 2′-O-methyladenosine-3′-phosphate; as is a 2′-O-methyladenosine-3′-phosphorothioate; c is a 2′-O-methylcytidine-3′-phosphate; cs is a 2′-O-methylcytidine-3′-phosphorothioate; g is a 2′-O-methylguanosine-3′-phosphate; gs is a 2′-O-methylguanosine-3′-phosphorothioate; u is a 2′-O-methyluridine-3′-phosphate; us is a 2′-O-methyluridine-3′-phosphorothioate; and s is a phosphorothioate linkage, thereby treating the subject.

In one embodiment, the subject is a human.

In one embodiment, the dsRNA agent, or salt thereof, is administered to the subject subcutaneously.

In one embodiment, the subcutaneous administration is subcutaneous injection.

In one embodiment, the nucleotide sequence of the sense strand differs by no more than 2 nucleotides from the nucleotide sequence 5′-gsascuuuCfaUfCfCfuggaaauaua-3′ (SEQ ID NO:35) and the nucleotide sequence of the antisense strand differs by no more than 2 nucleotides from the nucleotide sequence 5′-usAfsuauUfuCfCfaggaUfgAfaagucscsa-3′ (SEQ ID NO:36).

In one embodiment, the nucleotide sequence of the sense strand differs by no more than 1 nucleotide from the nucleotide sequence 5′-gsascuuuCfaUfCfCfuggaaauaua-3′ (SEQ ID NO:35) and the nucleotide sequence of the antisense strand differs by no more than 1 nucleotide from the nucleotide sequence 5′-usAfsuauUfuCfCfaggaUfgAfaagucscsa-3′ (SEQ ID NO:36).

In one embodiment, the nucleotide sequence of the sense strand comprises the nucleotide sequence 5′-gsascuuuCfaUfCfCfuggaaauaua-3′ (SEQ ID NO:35) and the nucleotide sequence of the antisense strand comprises the nucleotide sequence 5′-usAfsuauUfuCfCfaggaUfgAfaagucscsa-3′ (SEQ ID NO:36).

In one embodiment, the dsRNA agent, or salt thereof, is conjugated to a ligand as shown in the following schematic

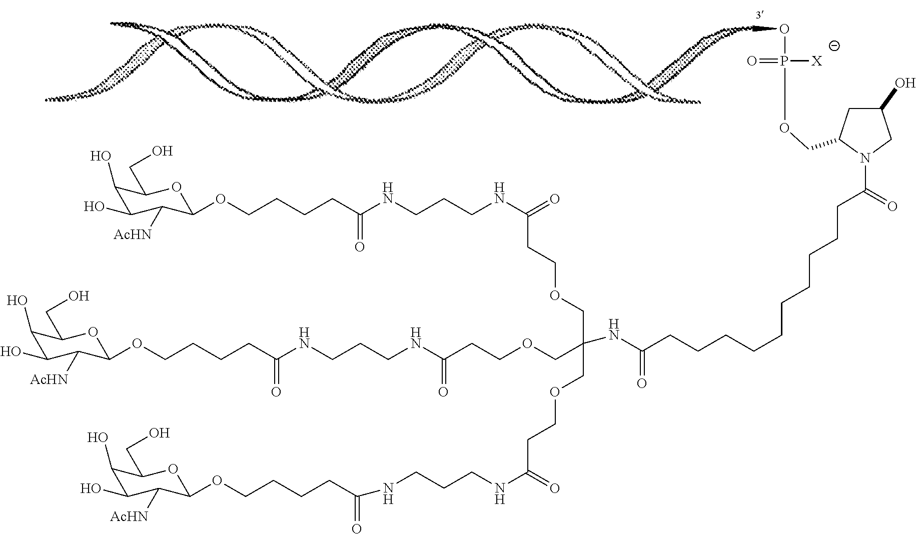

and, wherein X is O or S.

In one embodiment, the X is O.

In one embodiment, the dsRNA agent is in salt form.

In one embodiment, the dsRNA agent, or salt thereof, is administered to the subject in a pharmaceutical formulation.

In one embodiment, the methods further comprise administering an additional therapeutic to the subject.

### BRIEF DESCRIPTION OF THE DRAWINGS

FIG. 1 is a schematic of the endogenous pathways for oxalate synthesis.

### DETAILED DESCRIPTION OF THE INVENTION

The present invention is based, at least in part, on the discovery that agents that reduce oxalate levels, such as a nucleic acid inhibitor of lactate dehydrogenase A (LDHA), a nucleic acid inhibitor of hydroxyacid oxidase (HAO1) and/or a nucleic acid inhibitor of proline dehydrogenase 2 (PRODH2), can be used to treat subjects having or at risk of developing a non-primary hyperoxaluria disease or disorder, such as a subject having normal urinary oxalate levels, e.g., normal urinary calcium oxalate levels, or elevated urinary oxalate levels, e.g., elevated urinary calcium oxalate levels, e.g., supersaturated urinary calcium oxalate levels, e.g., a subject having a kidney stone disease, e.g., calcium oxalate kidney stone disease, such as recurrent calcium oxalate kidney stone disease.

Accordingly, the present invention provides methods for inhibiting the expression of hydroxyacid oxidase (HAO1) in a subject having a non-primary hyperoxaluria disease or disorder that would benefit from reduction in urinary oxalate, methods for reducing urinary oxalate levels in a subject having a non-primary hyperoxaluria disease or disorder that would benefit from reduction in urinary oxalate, and methods for treating a subject having having or at risk of developing a non-primary hyperoxaluria disease or disorder that would benefit from reduction in oxalate, and compositions comprising nucleic acid inhibitors, e.g., double stranded ribonucleic acid (dsRNA) agents or single stranded antisense polynucleotide agents targeting lactate dehydrogenase A (LDHA), hydroxyacid oxidase (HAO1) and/or proline dehydrogenase 2 (PRODH2).

The following detailed description discloses how to make and use compositions containing iRNAs to inhibit the expression of an HAO1 gene, an LDHA gene, a PRODH2 gene, and/or both an LDHA gene and an HAO1 gene, as well as compositions and methods for treating subjects having diseases and disorders that would benefit from inhibition and/or reduction of the expression of these genes.

### I. Definitions

In order that the present invention may be more readily understood, certain terms are first defined. In addition, it should be noted that whenever a value or range of values of a parameter are recited, it is intended that values and ranges intermediate to the recited values are also intended to be part of this invention.

The articles “a” and “an” are used herein to refer to one or to more than one (i.e., to at least one) of the grammatical object of the article. By way of example, “an element” means one element or more than one element, e.g., a plurality of elements.

The term “including” is used herein to mean, and is used interchangeably with, the phrase “including but not limited to”. The term “or” is used herein to mean, and is used interchangeably with, the term “and/or,” unless context clearly indicates otherwise.

The term “about” is used herein to mean within the typical ranges of tolerances in the art. For example, “about” can be understood as about 2 standard deviations from the mean. In certain embodiments, about means±10%. In certain embodiments, about means±5%. When about is present before a series of numbers or a range, it is understood that “about” can modify each of the numbers in the series or range.

The term “at least”, “no less than” or “or more” prior to a number or series of numbers is understood to include the number adjacent to the term “at least”, and all subsequent numbers or integers that could logically be included, as clear from context. For example, the number of nucleotides in a nucleic acid molecule must be an integer. For example, “at least 18 nucleotides of a 21 nucleotide nucleic acid molecule” means that 18, 19, 20, or 21 nucleotides have the indicated property. When at least is present before a series of numbers or a range, it is understood that “at least” can modify each of the numbers in the series or range.

As used herein, “no more than” or “or less” is understood as the value adjacent to the phrase and logical lower values or integers, as logical from context, to zero. For example, a duplex with an overhang of “no more than 2 nucleotides” has a 2, 1, or 0 nucleotide overhang. When “no more than” is present before a series of numbers or a range, it is understood that “no more than” can modify each of the numbers in the series or range.

In the event of a conflict between an indicated target site and the nucleotide sequence for a sense or antisense strand, the indicated sequence takes precedence.

In the event of a conflict between a chemical structure and a chemical name, the chemical structure takes precedence.

The term “hyperoxaluria”, as used herein, refers to a condition characterized by increased urinary excretion of oxalate. Generally, hyperoxaluria can be divided into two categories: primary and secondary hyperoxaluria.

Primary hyperoxaluria, as used herein, refers to autosomal recessive disorders of glyoxylate metabolism. Primary hyperoxaluria is the result of inherited enzyme deficiencies leading to increased endogenous oxalate synthesis. Primary hyperoxaluria can be divided into primary hyperoxaluria Type 1 (PH1); primary hyperoxaluria Type 2 (PH2); primary hyperoxaluria Type 3 (PH3); or primary hyperoxaluria Non-Type 1, Non-Type 2, Non-Type 3 (PH-Non-Type 1, Non-Type 2, Non-Type 3). PH1 is a hereditary disorder caused by mutations in alanine glyoxylate aminotransferase (AGT). PH2 is due to mutations in glyoxylate reductase/hydroxypyruvate reductase (GRHPR). PH3 is caused by mutations in HOGA1 (formerly DHDPSL). Subjects having PH-Non-Type 1, Non-Type 2, Non-Type 3 have clinical characteristics indistinguishable from type 1, 2, and 3, but with normal AGT, GRHPR, and HOGA1 liver enzyme activity, yet the etiology of the marked hyperoxaluria in such subjects remains to be elucidated.

A deficiency in either AGT or GRHPR activities results in an excess of glyoxylate and oxalate (see, e.g., Knight et al., (2011) Am J Physiol Renal Physiol 302(6): F688-F693). Therefore, inhibition of glycolate oxidase (HAO1) and proline dehydrogenase 2 (PRODH2) will reduce the level of glyoxylate. In addition, inhibition of LDHA expression and/or activity will decrease the level of excess oxalate. The buildup of oxalate in subjects having PH causes increased excretion of oxalate, which in turn results in renal and bladder stones. Stones cause urinary obstruction (often with severe and acute pain), secondary infection of urine and eventually kidney damage. Oxalate stones tend to be severe, resulting in relatively early kidney damage (e.g., onset in teenage years to early adulthood), which impairs the excretion of oxalate, leading to a further acceleration in accumulation of oxalate in the body. After the development of renal failure, patients may get deposits of oxalate in the bones, joints and bone marrow. Severe cases may develop hematological problems such as anaemia and thrombocytopaenia. The deposition of oxalate in the body is sometimes called “oxalosis” to be distinguished from “oxaluria” which refers to oxalate in the urine. Renal failure is a serious complication requiring treatment in its own right. Dialysis can control renal failure but tends to be inadequate to dispose of excess oxalate. Renal transplant is more effective and this is the primary treatment of severe hyperoxaluria. Liver transplantation (often in addition to renal transplant) may be able to control the disease by correcting the metabolic defect. In a proportion of patients with primary hyperoxaluria type 1, pyridoxine treatment (vitamin B6) may also decrease oxalate excretion and prevent kidney stone formation.

The term “a non-primary hyperoxaluria disease or disorder”, as used herein, refers to a disease, disorder or condition thereof, that is associated with oxalate metabolism, and would benefit from reduction in oxalate and/or from a decrease in the gene expression, replication, or protein activity of lactate dehydrogenase A (LDHA), hydroxyacid oxidase (HAO1) and/or proline dehydrogenase 2 (PRODH2).

The term “a non-primary hyperoxaluria disease or disorder,” as used herein, does not include primary hyperoxaluria, e.g., primary hyperoxaluria 1 (PH1), primary hyperoxaluria 2 (PH2), or primary hyperoxaluria 3 (PH3).

Subjects having a non-primary hyperoxaluria disease or disorder that would benefit from reduction in oxalate include subjects having an elevated level of oxalate, e.g., a mild hyperoxaluria condition, i.e., a urinary calcium oxalate excretion level of about 40 to about 60 mg/day, or a high hyperoxaluria condition, i.e., a urinary calcium oxalate excretion level of greater than about 60 mg/day. In one embodiment, subjects having a high hyperoxaluria condition have a supersaturation level of calcium oxalate, e.g., calcium oxalate (i.e., the concentration in urine is above the solubility of oxalate that drives crystallization and kidney stone formation). In other embodiments, subjects having a high hyperoxaluria condition do not have a supersaturation level of calcium oxalate, e.g., calcium oxalate. In some embodiments, subjects at risk of developing a non-primary hyperoxaluria disease or disorder, are subjects having a normal level of urinary oxalate excretion, i.e., a urinary oxalate excretion level of <40 mg/day and would still benefit from a reduction in oxalate.

Such subjects include those who suffer from a secondary hyperoxaluria, e.g., enteric hyperoxaluria, dietary hyperoxaluria, or idiopathic hyperoxaluria, a kidney stone disease, chronic kidney disease (CKD), end-stage renal disease (ESRD), coronary artery disease, cutaneous oxalate deposition, or ethylene glycol poisoning. Such subjects also include those who are planning to undergo kidney transplantation or have undergone kidney transplantation. In one embodiment, the subject suffers from a kidney stone disease, e.g., a calcium oxalate kidney stone disease, e.g., recurrent calcium oxalate kidney stone disease.

In certain embodiments, the methods of the invention reduce the level of urinary oxalate, e.g., urinary calcium oxalate, by about ≥20% from baseline as assessed in a 24-hour urinary oxalate analysis.

In certain embodiments, the methods of the invention reduce the level of urinary oxalate, e.g., urinary calcium oxalate, supersaturation from baseline as assessed in a 24-hour urinary oxalate analysis.

As used herein, the term “kidney stone disease” refers to a disease in which kidney stones (also called renal stones or urinary stones) form in one or both kidneys of the subject. Kidney stones are small, hard deposits which are made up of minerals or other compounds found in urine. Kidney stones vary in size, shape, and color. To be cleared from the body (or “passed”), the stones need to travel through ducts that carry urine from the kidneys to the bladder (ureters) and be excreted. Depending on their size, kidney stones generally take days to weeks to pass out of the body. There are four main types of kidney stones which are classified by the material they are made of Up to 75 percent of all kidney stones are composed primarily of calcium. Stones can also be made up of uric acid (a normal waste product), cystine (a protein building block), or struvite (a phosphate mineral). Stones form when there is more of the compound in the urine than can be dissolved. This imbalance can occur when there is an increased amount of the material in the urine, a reduced amount of liquid urine, or a combination of both. People are most likely to develop kidney stones between ages 40 and 60, though the stones can appear at any age. Research shows that 35 to 50 percent of people who have one kidney stone will develop additional stones, usually within 10 years of the first stone.

In one embodiment, the kidney stone disease is a calcium oxalate kidney stone disease. In another embodiment, the kidney stone disease is a non-calcium oxalate kidney stone disease.

In some embodiments, the kidney stone disease (either calcium oxalate kidney stone disease or non-calcium oxalate kidney stone disease) is non-recurrent kidney stone disease. In other embodiments, the kidney stone disease (either calcium oxalate kidney stone disease or non-calcium oxalate kidney stone disease) is recurrent kidney stone disease.

As used herein, the term “non-recurrent kidney stone disease” refers to kidney stone disease newly diagnosed in a subject, i.e., the subject was not previously diagnosed as having had kidney stone disease.

As used herein, the term “recurrent kidney stone disease” refers to kidney stone disease that returns in a subject that previously had kidney stone disease and was successfully treated for the disease (e.g., surgically treated to remove the kidney stone) or passed a kidney stone. Recurrent kidney stone disease may return at any time interval following treatment of the subject for kidney stone disease. In one embodiment, recurrent kidney stone disease is ≥2 stone events within a 5 year period.

“Chronic kidney disease” (“CKD”) or “chronic renal failure” (“CRF”), as defined by the Kidney Disease Outcomes Quality Initiative (KDOQI) of the National Kidney Foundation and the international guideline group Kidney Disease Improving Global Outcomes (KDIGO), is either kidney damage or a decreased glomerular filtration rate (GFR) of less than 60 mL/min/1.73 m2 for at least 3 months.

The different stages of CKD form a continuum. The stages of CKD are classified as: Stage 1: Kidney damage with normal or increased GFR (>90 mL/min/1.73 m²); Stage 2: Mild reduction in GFR (60-89 mL/min/1.73 m²); Stage 3a: Moderate reduction in GFR (45-59 mL/min/1.73 m²); Stage 3b: Moderate reduction in GFR (30-44 mL/min/1.73 m²); Stage 4: Severe reduction in GFR (15-29 mL/min/1.73 m²); Stage 5: Kidney failure (GFR <15 mL/min/1.73 m² or dialysis).

By itself, measurement of GFR may not be sufficient for identifying stage 1 and stage 2 CKD, because in those patients the GFR may in fact be normal or borderline normal. In such cases, the presence of one or more of the following markers of kidney damage can establish the diagnosis: Albuminuria (albumin excretion >30 mg/24 hr or albumin:creatinine ratio >30 mg/g [>3 mg/mmol]); Urine sediment abnormalities; Electrolyte and other abnormalities due to tubular disorders; Histologic abnormalities; Structural abnormalities detected by imaging; History of kidney transplantation in such cases

“End-stage renal disease” is the last stage of chronic kidney disease. Patients with end-stage renal disease will need dialysis or a kidney transplant in order to survive. In most cases, kidney failure is caused by other health problems, e.g., diabetes, or high blood pressure, that have done permanent damage to the kidneys over time.

“Secondary hyperoxaluria” results from over absorption of oxalate from the diet and is further characterized either as enteric, resulting from a chronic and unremediable underlying GI disorder associated with malabsorption, such as bariatric surgery complications or Crohn's disease, which predisposes patients to excess oxalate absorption, or idiopathic, meaning the underlying cause is unknown. Enteric hyperoxaluria is the more severe type of secondary hyperoxaluria. Secondary hyperoxaluria may also result from conditions underlying increased intestinal oxalate absorption, such as alterations in intestinal oxalate-degrading microorganisms, and genetic variations of intestinal oxalate transporters. Furthermore, hyperoxaluria may also occur following renal transplantation because of rapid clearance of accumulated oxalate.

In some embodiments, a non-primary hyperoxaluria disease or disorder is enteric hyperoxaluria. Enteric hyperoxaluria is the formation of calcium oxalate calculi in the urinary tract due to excessive absorption of oxalate from the colon, occurring as a result of intestinal bacterial overgrowth syndromes, fat malabsorption, chronic biliary or pancreatic disease, various intestinal surgical procedures, gastric bypass surgery, inflammatory bowel disease, or any medical condition that causes chronic diarrhea, e.g., Crohn's disease or ulcerative colitis).

In some embodiments, a non-primary hyperoxaluria disease or disorder is dietary hyperoxaluria, e.g., hyperoxaluria as a result of too much oxalate in the diet, e.g., from too much spinach, rhubarb, almonds, bulgur, millet, corn grits, soy flour, cornmeal, navy beans, etc.

In some embodiments, a non-primary hyperoxaluria disease or disorder is idiopathic hyperoxaluria. Subjects having idiopathic hyperoxaluria have above normal levels of urinary oxalate of unknown cause, but still develop stones.

In some embodiments, a non-primary hyperoxaluria disease or disorder is a calcium oxalate tissue deposition disease. For example, when glomerular filtration rate (GFR) drops below about 30-40 mL/min per 1.73 m², renal capacity to excrete calcium oxalate is significantly impaired. At this stage, calcium oxalate starts to deposit in extrarenal tissues. Calcium oxalate deposits may occur in the thyroid, breasts, kidneys, bones, bone marrow, myocardium, or cardiac conduction system. This leads to cardiomyopathy, heart block and other cardiac conduction defects, vascular diseases, retinopathy, synovitis, oxalate osteopathy and anemia that is noted to be resistant to treatment. The deposition of calcium oxalate mat be systemic or tissue specific.

Subjects having arthritis, sarcoidosis, end-stage renal disease are at risk of developing systemic calcium oxalate tissue deposition disease. Subjects at risk of developing tissue specific depositions in the kidney, for example, include subjects having medullary sponge kidney, nephrocalcinosis, renal tubular acidosis (RTA), and transplant recipients, e.g., kidney transplant recipients. In some embodiments, subjects at risk of developing tissue specific depositions include subjects having coronary artery disease or other vascular diseases, especially in patients with end-stage renal disease, HIV and other conditions where oxalate deposition occurs in plaques or in the vasculature.

In some embodiments, a non-primary hyperoxaluria disease or disorder is cutaneous oxalate deposition. Oxalate deposition in the skin can contribute to livedo reticularis, ulceration, and distal ischemia. In contrast to patients with primary hyperoxaluria, wherein oxalosis rarely occurs in the skin, patients with systemic oxalosis of chronic renal failure are more likely to present with extravascular calcified deposits of the skin, including dermal and subcutaneous nodules, tender subungual nodules, and skin-colored to yellow macules and papules usually in an acral distribution or on the face. In some embodiments, the non-primary hyperoxaluria disease or disorder is cutaneous oxalate deposition in the setting of dialysis.

In some embodiments, a non-primary hyperoxaluria disease or disorder is ethylene glycol poisoning. Ethylene glycol is an important cause of metabolic acidosis and subsequent acute renal failure, and the toxicity results from the depressant effects of ethylene glycol on the central nervous system. Specifically, metabolic acidosis and renal failure are caused by the conversion of ethylene glycol to noxious metabolites. Oxidative reactions convert ethylene glycol to glycolaldehyde, and then to glycolic acid, which is the major cause of metabolic acidosis. Both of these steps promote the production of lactate from pyruvate. The conversion of glycolic acid to glyoxylic acid proceeds slowly, further increasing the serum concentration of glycolic acid. Glyoxylic acid is eventually converted to oxalic acid and glycine. Oxalic acid does not contribute to the metabolic acidosis, but it is deposited as calcium oxalate crystals in many tissues.

As used herein, a “subject” is an animal, such as a mammal, including a primate (such as a human, a non-human primate, e.g., a monkey, and a chimpanzee), a non-primate (such as a cow, a pig, a camel, a llama, a horse, a goat, a rabbit, a sheep, a hamster, a guinea pig, a cat, a dog, a rat, a mouse, a horse, and a whale), or a bird (e.g., a duck or a goose). In one embodiment, a subject is a human subject

As used herein, the terms “treating” or “treatment” refer to a beneficial or desired result, such as inhibiting oxalate accumulation and/or lowering urinary excretion levels of oxalate in a subject. The terms “treating” or “treatment” also include, but are not limited to, alleviation or amelioration of one or more symptoms of a non-primary hyperoxaluria disease or disorder, such as, e.g., slowing the course of the disease; reducing the severity of later-developing disease; and/or preventing further oxalate tissue deposition. “Treatment” can also mean prolonging survival as compared to expected survival in the absence of treatment.

The term “lower” in the context of a disease marker or symptom refers to a statistically significant decrease in such level. The decrease can be, for example, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or more and is a decrease to a level accepted as within the range of normal for an individual without such disorder.

As used herein, “prevention” or “preventing,” when used in reference to a disease refers to a reduction in the likelihood that a subject will develop a symptom associated with such disease, disorder, or condition, e.g., oxalate accumulation or stone formation. The likelihood of, e.g., oxalate accumulation or stone formation, is reduced, for example, when an individual having one or more risk factors for stone formation either fails to develop stones or develops stones with less severity relative to a population having the same risk factors and not receiving treatment as described herein. The failure to develop a disease, or the reduction in the development of a symptom associated with such a disease, disorder or condition (e.g., by at least about 10% on a clinically accepted scale for that disease or disorder), or the exhibition of delayed symptoms delayed (e.g., by days, weeks, months or years) is considered effective prevention.

“Therapeutically effective amount,” as used herein, is intended to include the amount of an inhibitor that, when administered to a subject having a non-primary hyperoxaluria disease, is sufficient to effect treatment of the disease (e.g., by diminishing, ameliorating or maintaining the existing disease or one or more symptoms of disease). The “therapeutically effective amount” may vary depending on the inhibitor, how the inhibitor is administered, the disease and its severity and the history, age, weight, family history, genetic makeup, the types of preceding or concomitant treatments, if any, and other individual characteristics of the subject to be treated.

“Prophylactically effective amount,” as used herein, is intended to include the amount of an inhibitor that, when administered to a subject having a non-primary hyperoxaluria disease, is sufficient to prevent or ameliorate the disease or one or more symptoms of the disease. Ameliorating the disease includes slowing the course of the disease or reducing the severity of later-developing disease. The “prophylactically effective amount” may vary depending on the inhibitor, how the inhibitor is administered, the degree of risk of disease, and the history, age, weight, family history, genetic makeup, the types of preceding or concomitant treatments, if any, and other individual characteristics of the patient to be treated.

A “therapeutically-effective amount” or “prophylactically effective amount” also includes an amount of an inhibitor that produces some desired local or systemic effect at a reasonable benefit/risk ratio applicable to any treatment. Inhibitors employed in the methods of the present invention may be administered in a sufficient amount to produce a reasonable benefit/risk ratio applicable to such treatment.

In the methods of the invention which include administering to a subject a pharmaceutical composition comprising a first dsRNA agent targeting LDHA and a second dsRNA agent targeting HAO1, the therapeutically effective amount of the first dsRNA agent may be the same or different than the therapeutically effective amount of the second dsRNA agent. Similarly, in the methods of the invention which include administering to a subject a pharmaceutical composition comprising a first dsRNA agent targeting LDHA and a second dsRNA agent targeting HAO1, the prophylacticly effective amount of the first dsRNA agent may be the same or different than the prophylactically effective amount of the second dsRNA agent.

In addition, in the methods of the invention which include administering to a subject a pharmaceutical composition comprising a first single stranded antisense polynucleotide agent targeting LDHA and a second single stranded antisense polynucleotide agent targeting HAO1, the therapeutically effective amount of the first single stranded antisense polynucleotide agent may be the same or different than the therapeutically effective amount of the second single stranded antisense polynucleotide agent. Similarly, in the methods of the invention which include administering to a subject a pharmaceutical composition comprising a first single stranded antisense polynucleotide agent targeting LDHA and a second single stranded antisense polynucleotide agent targeting HAO1, the prophylacticly effective amount of the first single stranded antisense polynucleotide agent may be the same or different than the prophylactically effective amount of the second single stranded antisense polynucleotide agent.

As used herein, the term a “nucleic acid inhibitor” includes iRNA agents and antisense polynucleotide agents.

The terms “iRNA”, “RNAi agent,” “iRNA agent,” “RNA interference agent” as used interchangeably herein, refer to an agent that contains RNA as that term is defined herein, and which mediates the targeted cleavage of an RNA transcript via an RNA-induced silencing complex (RISC) pathway. RNA interference (RNAi) is a process that directs the sequence-specific degradation of mRNA. RNAi modulates, e.g., inhibits, the expression of LDHA, PRODH2 and/or HAO1 in a cell, e.g., a cell within a subject, such as a subject suffering from a non-primary hyperoxaluria disease or disorder.

In one embodiment, an RNAi agent of the disclosure includes a single stranded RNAi that interacts with a target RNA sequence, e.g., an LDHA, PRODH2, and/or HAO1 target mRNA sequence, to direct the cleavage of the target RNA. Without wishing to be bound by theory it is believed that long double stranded RNA introduced into cells is broken down into double-stranded short interfering RNAs (siRNAs) comprising a sense strand and an antisense strand by a Type III endonuclease known as Dicer (Sharp et al. (2001) Genes Dev. 15:485). Dicer, a ribonuclease-III-like enzyme, processes these dsRNA into 19-23 base pair short interfering RNAs with characteristic two base 3′ overhangs (Bernstein, et al., (2001) Nature 409:363). These siRNAs are then incorporated into an RNA-induced silencing complex (RISC) where one or more helicases unwind the siRNA duplex, enabling the complementary antisense strand to guide target recognition (Nykanen, et al., (2001) Cell 107:309). Upon binding to the appropriate target mRNA, one or more endonucleases within the RISC cleave the target to induce silencing (Elbashir, et al., (2001) Genes Dev. 15:188). Thus, in one aspect the disclosure relates to a single stranded RNA (ssRNA) (the antisense strand of a siRNA duplex) generated within a cell and which promotes the formation of a RISC complex to effect silencing of the target gene, i.e., an LDHA, PRODH2 and/or HAO1 gene. Accordingly, the term “siRNA” is also used herein to refer to an RNAi as described above.

In another embodiment, the RNAi agent may be a single-stranded RNA that is introduced into a cell or organism to inhibit a target mRNA. Single-stranded RNAi agents bind to the RISC endonuclease, Argonaute 2, which then cleaves the target mRNA. The single-stranded siRNAs are generally 15-30 nucleotides and are chemically modified. The design and testing of single-stranded RNAs are described in U.S. Pat. No. 8,101,348 and in Lima et al., (2012) Cell 150:883-894, the entire contents of each of which are hereby incorporated herein by reference. Any of the antisense nucleotide sequences described herein may be used as a single-stranded siRNA as described herein or as chemically modified by the methods described in Lima et al., (2012) Cell 150:883-894.

In another embodiment, a “RNAi agent” for use in the compositions and methods of the disclosure is a double stranded RNA and is referred to herein as a “double stranded RNAi agent,” “double stranded RNA (dsRNA) molecule,” “dsRNA agent,” or “dsRNA”. The term “dsRNA” refers to a complex of ribonucleic acid molecules, having a duplex structure comprising two anti-parallel and substantially complementary nucleic acid strands, referred to as having “sense” and “antisense” orientations with respect to a target RNA, i.e., an LDHA, PRODH2 and/or HAO1 gene. In some embodiments of the disclosure, a double stranded RNA (dsRNA) triggers the degradation of a target RNA, e.g., an mRNA, through a post-transcriptional gene-silencing mechanism referred to herein as RNA interference or RNAi.

In yet another embodiment, an “iRNA” for use in the compositions and methods of the invention is a “dual targeting RNAi agent.” The term “dual targeting RNAi agent” refers to a molecule comprising a first dsRNA agent comprising a complex of ribonucleic acid molecules, having a duplex structure comprising two anti-parallel and substantially complementary nucleic acid strands, referred to as having “sense” and “antisense” orientations with respect to a first target RNA, i.e., an LDHA gene, covalently attached to a molecule comprising a second dsRNA agent comprising a complex of ribonucleic acid molecules, having a duplex structure comprising two anti-parallel and substantially complementary nucleic acid strands, referred to as having “sense” and “antisense” orientations with respect to a second target RNA, i.e., an HAO1 gene. In some embodiments of the invention, a dual targeting RNAi agent triggers the degradation of the first and the second target RNAs, e.g., mRNAs, through a post-transcriptional gene-silencing mechanism referred to herein as RNA interference or RNAi.

The terms “polynucleotide agent,” “antisense polynucleotide agent” “antisense compound”, and “agent” as used interchangeably herein, refer to an agent comprising a single-stranded oligonucleotide that contains RNA as that term is defined herein, and which targets nucleic acid molecules encoding LDHA, PRODH2 and/or HAO1 (e.g., mRNA encoding LDHA, PRODH2 and/or HAO1). The antisense polynucleotide agents specifically bind to the target nucleic acid molecules via hydrogen bonding (e.g., Watson-Crick, Hoogsteen, or reversed Hoogsteen hydrogen bonding) and interfere with the normal function of the targeted nucleic acid (e.g., by an antisense mechanism of action). This interference with or modulation of the function of a target nucleic acid by the polynucleotide agents of the present invention is referred to as “antisense inhibition.” The functions of the target nucleic acid molecule to be interfered with may include functions such as, for example, translocation of the RNA to the site of protein translation, translation of protein from the RNA, splicing of the RNA to yield one or more mRNA species, and catalytic activity which may be engaged in or facilitated by the RNA.

As used herein, “target sequence” refers to a contiguous portion of the nucleotide sequence of an mRNA molecule formed during the transcription of an LDHA gene, a PRODH2 gene, or an HAO1 gene, including mRNA that is a product of RNA processing of a primary transcription product.

In one embodiment, the target portion of the sequence will be at least long enough to serve as a substrate for iRNA-directed cleavage at or near that portion of the nucleotide sequence of an mRNA molecule formed during the transcription of an LDHA gene. In another embodiment, the target portion of the sequence will be at least long enough to serve as a substrate for iRNA-directed cleavage at or near that portion of the nucleotide sequence of an mRNA molecule formed during the transcription of a PRODH2 gene. In another embodiment, the target portion of the sequence will be at least long enough to serve as a substrate for iRNA-directed cleavage at or near that portion of the nucleotide sequence of an mRNA molecule formed during the transcription of an HAO1 gene.

The target sequence of an LDHA gene, a PRODH2 gene or an HAO1 gene may be from about 19-36 nucleotides in length, e.g., about 19-30 nucleotides in length. For example, the target sequence can be about 19-30 nucleotides, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 nucleotides in length. In certain embodiments, the target sequence is 19-23 nucleotides in length, optionally 21-23 nucleotides in length. Ranges and lengths intermediate to the above recited ranges and lengths are also contemplated to be part of the disclosure.

In aspects in which a first dsRNA agent targeting LDHA and a second dsRNA agent targeting HAO1 are covalently attached (i.e., a dual targeting RNAi agent), the length of the LDHA target sequence may be the same as the HAO1 target sequence or different.

A target sequence may be from about 4-50 nucleotides in length, e.g., 8-45, 10-45, 10-40, 10-35, 10-30, 10-20, 11-45, 11-40, 11-35, 11-30, 11-20, 12-45, 12-40, 12-35, 12-30, 12-25, 12-20, 13-45, 13-40, 13-35, 13-30, 13-25, 13-20, 14-45, 14-40, 14-35, 14-30, 14-25, 14-20, 15-45, 15-40, 15-35, 15-30, 15-25, 15-20, 16-45, 16-40, 16-35, 16-30, 16-25, 16-20, 17-45, 17-40, 17-35, 17-30, 17-25, 17-20, 18-45, 18-40, 18-35, 18-30, 18-25, 18-20, 19-45, 19-40, 19-35, 19-30, 19-25, 19-20, e.g., 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 contiguous nucleotides of the nucleotide sequence of an mRNA molecule formed during the transcription of an LDHA gene and/or an HAO1 gene. Ranges and lengths intermediate to the above recited ranges and lengths are also contemplated to be part of the invention.

The terms “complementary,” “fully complementary” and “substantially complementary” are used herein with respect to the base matching between a nucleic acid inhibitor and a target sequence. The term“complementarity” refers to the capacity for pairing between nucleobases of a first nucleic acid and a second nucleic acid.

As used herein, a nucleic acid inhibitor that is “substantially complementary to at least part of” a messenger RNA (mRNA) refers to a nucleic acid inhibitor that is substantially complementary to a contiguous portion of the mRNA of interest (e.g., an mRNA encoding LDHA, an mRNA encoding PRODH2, and/or an mRNA encoding HAO1). For example, a polynucleotide is complementary to at least a part of an HAO1 mRNA if the sequence is substantially complementary to a non-interrupted portion of an mRNA encoding HAO1.

As used herein, the term “region of complementarity” refers to the region of the nucleic acid inhibito that is substantially complementary to a sequence, for example a target sequence, e.g., an LDHA nucleotide sequence, a PRODH2 nucleotide sequence and/or an HAO1 nucleotide sequence, as defined herein. Where the region of complementarity is not fully complementary to the target sequence, the mismatches can be in the internal or terminal regions of the molecule. Generally, the most tolerated mismatches are in the terminal regions, e.g., within 5, 4, 3, or 2 nucleotides of the 5′- and/or 3′-terminus of the polynucleotide.

As used herein, and unless otherwise indicated, the term “complementary,” when used to describe a first nucleotide sequence in relation to a second nucleotide sequence, refers to the ability of a polynucleotide comprising the first nucleotide sequence to hybridize and form a duplex structure under certain conditions with the second nucleotide sequence, as will be understood by the skilled person. Such conditions can, for example, be stringent conditions, where stringent conditions can include: 400 mM NaCl, 40 mM PIPES pH 6.4, 1 mM EDTA, 50° C. or 70° C. for 12-16 hours followed by washing (see, e.g., “Molecular Cloning: A Laboratory Manual, Sambrook, et al. (1989) Cold Spring Harbor Laboratory Press). Other conditions, such as physiologically relevant conditions as can be encountered inside an organism, can apply. The skilled person will be able to determine the set of conditions most appropriate for a test of complementarity of two sequences in accordance with the ultimate application of the nucleotides.

Complementary sequences include those nucleotide sequences of a nucleic acid inhibitor of the invention that base-pair to a second nucleotide sequence over the entire length of one or both nucleotide sequences. Such sequences can be referred to as “fully complementary” with respect to each other herein. However, where a first sequence is referred to as “substantially complementary” with respect to a second sequence herein, the two sequences can be fully complementary, or they can form one or more, but generally not more than 5, 4, 3 or 2 mismatched base pairs upon hybridization for a duplex up to 30 base pairs, while retaining the ability to hybridize under the conditions most relevant to their ultimate application, e.g., inhibition of target gene expression.

“Complementary” sequences, as used herein, can also include, or be formed entirely from, non-Watson-Crick base pairs and/or base pairs formed from non-natural and modified nucleotides, in so far as the above requirements with respect to their ability to hybridize are fulfilled. Such non-Watson-Crick base pairs include, but are not limited to, G:U Wobble or Hoogsteen base pairing. As used herein, the term “strand comprising a sequence” refers to an oligonucleotide comprising a chain of nucleotides that is described by the sequence referred to using the standard nucleotide nomenclature.

“G,” “C,” “A,” “T” and “U” each generally stand for a nucleotide that contains guanine, cytosine, adenine, thymidine and uracil as a base, respectively. However, it will be understood that the terms “deoxyribonucleotide”, “ribonucleotide” and “nucleotide” can also refer to a modified nucleotide, as further detailed below, or a surrogate replacement moiety (see, e.g., Table 1). The skilled person is well aware that guanine, cytosine, adenine, and uracil can be replaced by other moieties without substantially altering the base pairing properties of an oligonucleotide comprising a nucleotide bearing such replacement moiety. For example, without limitation, a nucleotide comprising inosine as its base can base pair with nucleotides containing adenine, cytosine, or uracil. Hence, nucleotides containing uracil, guanine, or adenine can be replaced in the nucleotide sequences of the agents featured in the invention by a nucleotide containing, for example, inosine. In another example, adenine and cytosine anywhere in the oligonucleotide can be replaced with guanine and uracil, respectively to form G-U Wobble base pairing with the target mRNA. Sequences containing such replacement moieties are suitable for the compositions and methods featured in the invention.

A “nucleoside” is a base-sugar combination. The “nucleobase” (also known as “base”) portion of the nucleoside is normally a heterocyclic base moiety. “Nucleotides” are nucleosides that further include a phosphate group covalently linked to the sugar portion of the nucleoside. For those nucleosides that include a pentofuranosyl sugar, the phosphate group can be linked to the 2′, 3′ or 5′ hydroxyl moiety of the sugar. “Polynucleotides,” also referred to as “oligonucleotides,” are formed through the covalent linkage of adjacent nucleosides to one another, to form a linear polymeric oligonucleotide. Within the polynucleotide structure, the phosphate groups are commonly referred to as forming the internucleoside linkages of the polynucleotide.

In general, the majority of nucleotides of the nucleic acid inhibitors are ribonucleotides, but as described in detail herein, the inhibitors may also include one or more non-ribonucleotides, e.g., a deoxyribonucleotide. In addition, as used in this specification, a “nucleic acid inhibitor” may include nucleotides (e.g., ribonucleotides or deoxyribonucleotides) with chemical modifications; a nucleic acid inhibitor may include substantial modifications at multiple nucleotides.

As used herein, the term “modified nucleotide” refers to a nucleotide having, independently, a modified sugar moiety, a modified internucleotide linkage, and/or modified nucleobase. Thus, the term modified nucleotide encompasses substitutions, additions or removal of, e.g., a functional group or atom, to internucleoside linkages, sugar moieties, or nucleobases. The modifications suitable for use in the nucleic acid inhibitors of the invention include all types of modifications disclosed herein or known in the art. Any such modifications, as used in nucleotides, are encompassed by “nucleic acid inhibitor” for the purposes of this specification and claims.

The term “LDHA” (used interchangeable herein with the term “Ldha”), also known as Cell Proliferation-Inducing Gene 19 Protein, Renal Carcinoma Antigen NY-REN-59, LDH Muscle Subunit, EC 1.1.1.27 4 61, LDH-A, LDH-M, Epididymis Secretory Sperm Binding Protein Li 133P, L-Lactate Dehydrogenase A Chain, Proliferation-Inducing Gene 19, Lactate Dehydrogenase M, HEL-S-133P, EC 1.1.1, GSD11, PIG19, and LDHM, refers to the well known gene encoding a lactate dehydrogenase A from any vertebrate or mammalian source, including, but not limited to, human, bovine, chicken, rodent, mouse, rat, porcine, ovine, primate, monkey, and guinea pig, unless specified otherwise.

The term also refers to fragments and variants of native LDHA that maintain at least one in vivo or in vitro activity of a native LDHA. The term encompasses full-length unprocessed precursor forms of LDHA as well as mature forms resulting from post-translational cleavage of the signal peptide and forms resulting from proteolytic processing.

The sequence of a human LDHA mRNA transcript can be found at, for example, GenBank Accession No. GI: 207028493 (NM_001135239.1; SEQ ID NO: 1), GenBank Accession No. GI: 260099722 (NM_001165414.1; SEQ ID NO:3), GenBank Accession No. GI: 260099724 (NM_001165415.1; SEQ ID NO: 5), GenBank Accession No. GI: 260099726 (NM_001165416.1; SEQ ID NO:7), GenBank Accession No. GI: 207028465 (NM_005566.3; SEQ ID NO:9); the sequence of a mouse LDHA mRNA transcript can be found at, for example, GenBank Accession No. GI: 257743038 (NM_001136069.2; SEQ ID NO: 11), GenBank Accession No. GI: 257743036 (NM_010699.2; SEQ ID NO: 13); the sequence of a rat LDHA mRNA transcript can be found at, for example, GenBank Accession No. GI: 8393705 (NM_017025.1; SEQ ID NO: 15); and the sequence of a monkey LDHA mRNA transcript can be found at, for example, GenBank Accession No. GI: 402766306 (NM_001257735.2; SEQ ID NO: 17), GenBank Accession No. GI: 545687102 (NM_001283551.1; SEQ ID NO:19).

Additional examples of LDHA mRNA sequences are readily available using publicly available databases, e.g., GenBank, UniProt, and OMIM.

The term “LDHA” as used herein also refers to a particular polypeptide expressed in a cell by naturally occurring DNA sequence variations of the LDHA gene, such as a single nucleotide polymorphism in the LDHA gene. Numerous SNPs within the LDHA gene have been identified and may be found at, for example, NCBI dbSNP (see, e.g., www.ncbi.nlm.nih.gov/snp).

As used herein, the term “HAO1” refers to the well known gene encoding the enzyme hydroxyacid oxidase 1 from any vertebrate or mammalian source, including, but not limited to, human, bovine, chicken, rodent, mouse, rat, porcine, ovine, primate, monkey, and guinea pig, unless specified otherwise. Other gene names include GO, GOX, GOX1, HAO, and HAOX1. The protein is also known as glycolate oxidase and (S)-2-hydroxy-acid oxidase.

The term also refers to fragments and variants of native HAO1 that maintain at least one in vivo or in vitro activity of a native HAO1. The term encompasses full-length unprocessed precursor forms of HAO1 as well as mature forms resulting from post-translational cleavage of the signal peptide and forms resulting from proteolytic processing. The sequence of a human HAO1 mRNA transcript can be found at, for example, GenBank Accession No. GI: 11184232 (NM_017545.2; SEQ ID NO:21); the sequence of a monkey HAO1 mRNA transcript can be found at, for example, GenBank Accession No. GI:544464345 (XM_005568381.1; SEQ ID NO:23); the sequence of a mouse HAO1 mRNA transcript can be found at, for example, GenBank Accession No. GI: 133893166 (NM_010403.2; SEQ ID NO:25); and the sequence of a rat HAO1 mRNA transcript can be found at, for example, GenBank Accession No. GI: 166157785 (NM_001107780.2; SEQ ID NO:27).

The term “HAO1,” as used herein, also refers to naturally occurring DNA sequence variations of the HAO1 gene, such as a single nucleotide polymorphism (SNP) in the HAO1 gene. Exemplary SNPs may be found in the NCBI dbSNP Short Genetic Variations database available at www.ncbi.nlm.nih.gov/projects/SNP.

As used herein, “proline dehydrogenase 2,” used interchangeably with the term “PRODH2,” refers to the enzyme which catalyzes the first step in the catabolism of trans-4-hydroxy-L-proline, an amino acid derivative obtained through food intake and collagen turnover. Glyoxylate is one of the downstream products of hydroxyproline catabolism, which in people with disorders of glyoxalate metabolism can lead to an increase in oxalate levels and the formation of calcium-oxalate kidney stones. PRODH2 is also known as proline dehydrogenase, HYPDH, HSPOX1, and hydroxyproline dehydrogenase.

The sequence of a human PRODH2 mRNA transcript can be found at, for example, GenBank Accession No. GI: 1818882103 (NM_021232.2; SEQ ID NO:4641; reverse complement, SEQ ID NO: 4642). The sequence of mouse PRODH2 mRNA can be found at, for example, GenBank Accession No. GI: 142372879 (NM_019546.5; SEQ ID NO:4643; reverse complement, SEQ ID NO: 4644). The sequence of rat PRODH2 mRNA can be found at, for example, GenBank Accession No. GI: 198278487 (NM_001038588.1; SEQ ID NO:4645; reverse complement, SEQ ID NO: 4646). The sequence of Macaca fascicularis PRODH2 mRNA can be found at, for example, GenBank Accession No. GI: 982316449 (XM_005588902.2; SEQ ID NO: 4647; reverse complement, SEQ ID NO: 4648). The sequence of Macaca mulatta PRODH2 mRNA can be found at, for example, GenBank Accession No. GI: 1622893613 (XM_015123711.2; SEQ ID NO: 4649; reverse complement, SEQ ID NO: 4650).

Additional examples of PRODH2 mRNA sequences are readily available through publicly available databases, e.g., GenBank, UniProt, OMIM, and the Macaca genome project web site.

Further information on PRODH2 can be found, for example, at www.ncbi.nlm.nih.gov/gene/?term=PRODH2.

The entire contents of each of the foregoing GenBank Accession numbers and the Gene database numbers are incorporated herein by reference as of the date of filing this application.

The term PRODH2, as used herein, also refers to variations of the PRODH2 gene including variants provided in the SNP database. Numerous sequence variations within the PRODH2 gene have been identified and may be found at, for example, NCBI dbSNP and UniProt (see, e.g., www.ncbi.nlm.nih.gov/snp/?term=PRODH2, the entire contents of which is incorporated herein by reference as of the date of filing this application.

### II. Methods of the Invention

The present invention provides a method for inhibiting the expression of hydroxyacid oxidase (HAO1) in a subject, e.g., a human subject, having a non-primary hyperoxaluria disease or disorder that would benefit from reduction in urinary oxalate. The present invention also provides a method for reducing urinary oxalate levels, e.g., urinary oxalate is urinary calcium oxalate, e.g., urinary calcium oxalate supersaturation in a subject, e.g., a human subject, having a non-primary hyperoxaluria disease or disorder that would benefit from reduction in urinary oxalate. In addition, the present invention provides a method for treating a subject, e.g., a human subject, having a non-primary hyperoxaluria disease or disorder that would benefit from reduction in oxalate. The methods include administering, e.g., subcutaneously administering, e.g., subcutaneous injection, to the subject a fixed dose of about 200 mg to about 600 mg, e.g., about 284 mg or about 567 mg, of a double stranded ribonucleic acid (dsRNA) agent, or salt thereof, which inhibits the expression of of HAO1, thereby inhibiting the expression of HAO1 in the subject.

In other aspects, the present invention also provides a method for treating a subject having a non-primary hyperoxaluria disease or disorder that would benefit from reduction in oxalate. The method includes administering to the subject a therapeutically effective amount of a nucleic acid inhibitor of hydroxyacid oxidase (HAO1) and/or a nucleic acid inhibitor of Proline Dehydrogenase 2 (PRODH2), thereby treating the subject having the non-primary hyperoxaluria disease or disorder that would benefit from reduction in oxalate.

In addition, the present invention also provides a method of treating a subject at risk of developing a non-primary hyperoxaluria disease or disorder that would benefit from reduction in oxalate. The method includes administering to the subject a therapeutically effective amount of a nucleic acid inhibitor of lactate dehydrogenase A (LDHA), a nucleic acid inhibitor of hydroxyacid oxidase (HAO1), and/or a nucleic acid inhibitor of Proline Dehydrogenase 2 (PRODH2), thereby treating the subject at risk of developing the non-primary hyperoxaluria disease or disorder that would benefit from reduction in oxalate.

Subjects having a non-primary hyperoxaluria disease or disorder that would benefit from reduction in oxalate include subjects having an elevated level of oxalate, e.g., a mild hyperoxaluria condition, i.e., a urinary calcium oxalate excretion level of about 40 to about 60 mg/day, or a high hyperoxaluria condition, i.e., a urinary calcium oxalate excretion level of greater than about 60 mg/day. In one embodiment, subjects having a high hyperoxaluria condition have a supersaturation level of calcium oxalate, e.g., calcium oxalate (i.e., the concentration in urine is above the solubility of oxalate that drives crystallization and kidney stone formation). In other embodiments, subjects having a high hyperoxaluria condition do not have a supersaturation level of calcium oxalate, e.g., calcium oxalate. In some embodiments, subjects at risk of developing a non-primary hyperoxaluria disease or disorder, are subjects having a normal level of urinary oxalate excretion, i.e., a urinary oxalate excretion level of <40 mg/day and would still benefit from a reduction in oxalate.

Such subjects include those who suffer from a secondary hyperoxaluria, e.g., enteric hyperoxaluria, dietary hyperoxaluria, or idiopathic hyperoxaluria, a kidney stone disease, chronic kidney disease (CKD), end-stage renal disease (ESRD), coronary artery disease, cutaneous oxalate deposition, or ethylene glycol poisoning. Such subjects also include those who are planning to undergo kidney transplantation or have undergone kidney transplantation.

In the methods of the present invention, subjects having a non-primary hyperoxaluria disease or disorder that would benefit from reduction in oxalate do not have primary hyperoxaluria (PH), i.e., PH1, PH2, or PH3.

In some embodiments, the non-primary hyperoxaluria disease or disorder is a kidney stone disease, e.g., calcium oxalate kidney stone disease, e.g., recurrent calcium oxalate kidney stone disease.

Administration of the dsRNA agent, or salt thereof, is to a subject may be repeated on a regular basis, for example, at an interval of once every three months, or once every six months.

In one embodiment, the dsRNA agent, or salt thereof, is administered to the subject at an interval of once every six months.

In other embodiment, the dsRNA agent, or salt thereof, is administered to the subject initially, at three months, and every six months thereafter.

Administration of the dsRNA, or salt thereof, to the subject may, e.g., reduce urinary oxalate levels, e.g., urinary calcium oxalate, urinary calcium oxalate supersaturation, e.g., by about ≥20% from baseline as assessed in a 24-hour urinary oxalate analysis, and/or reduce clinical and radiographic kidney stone events.

When the subject to be treated is a mammal such as a human, the nucleic acid inhibitor can be administered by any means known in the art including, but not limited to oral, intraperitoneal, or parenteral routes, including intracranial (e.g., intraventricular, intraparenchymal and intrathecal), intravenous, intramuscular, subcutaneous, transdermal, airway (aerosol), nasal, rectal, and topical (including buccal and sublingual) administration. In certain embodiments, the compositions are administered by intravenous infusion or injection. In certain embodiments, the compositions are administered by subcutaneous injection.

In some embodiments, the administration is via a depot injection. A depot injection may release the nucleic acid inhibitor in a consistent way over a prolonged time period. Thus, a depot injection may reduce the frequency of dosing needed to obtain a desired effect, e.g., a desired inhibition of LDHA or HAO1 or PRODH2, or a desired inhibition of both LDHA and HAO1, or a therapeutic or prophylactic effect. A depot injection may also provide more consistent serum concentrations. Depot injections may include subcutaneous injections or intramuscular injections. In certain embodiments, the depot injection is a subcutaneous injection.

In some embodiments, the administration is via a pump. The pump may be an external pump or a surgically implanted pump. In certain embodiments, the pump is a subcutaneously implanted osmotic pump. In other embodiments, the pump is an infusion pump. An infusion pump may be used for intravenous, subcutaneous, arterial, or epidural infusions. In certain embodiments, the infusion pump is a subcutaneous infusion pump. In other embodiments, the pump is a surgically implanted pump that delivers the nucleic acid inhibitor to the liver.

A nucleic acid inhibitor of the invention, e.g., the dsRNA agent, or salt thereof, may be present in a pharmaceutical composition, such as in a suitable buffer solution. The buffer solution may comprise acetate, citrate, prolamine, carbonate, or phosphate, or any combination thereof. In one embodiment, the buffer solution is phosphate buffered saline (PBS). The pH and osmolarity of the buffer solution containing the iRNA can be adjusted such that it is suitable for administering to a subject.

Alternatively, a nucleic acid inhibitor of the invention may be administered as a pharmaceutical composition, such as a dsRNA liposomal formulation.

The mode of administration may be chosen based upon whether local or systemic treatment is desired and based upon the area to be treated. The route and site of administration may be chosen to enhance targeting.

The methods (and uses) of the invention include administering to the subject, e.g., a human, a therapeutically effective amount of a nucleic acid inhibitor, e.g., a dsRNA agent, a dual targeting iRNA agent, a single stranded antisense polynucleotide agent, or a pharmaceutical composition comprising a nucleic acid inhibitor, e.g., a dsRNA, a pharmaceutical composition comprising a dual targeting RNAi agent, a pharmaceutical composition of the invention comprising a first dsRNA agent that inhibits expression of LDHA and a second dsRNA agent that inhibits expression of HAO1, or a pharmaceutical composition of the invention comprising a single stranded antisense polynucleotide agent.

Subjects that would benefit from the methods of the invention include subjects having or at risk of developing a non-primary hyperoxaluria disease.

In the methods (and uses) of the invention which comprise administering to a subject a first nucleic acid inhibitor, such as a dsRNA agent targeting LDHA and a second dsRNA agent targeting HAO1, the first and second nucleic acid inhibitor may be formulated in the same composition or different compositions and may administered to the subject in the same composition or in separate compositions.

The nucleic acid inhibitor may be administered to the subject at a dose of about 0.1 mg/kg to about 50 mg/kg. Typically, a suitable dose will be in the range of about 0.1 mg/kg to about 5.0 mg/kg, such as about 0.3 mg/kg and about 3.0 mg/kg.

In the methods (and uses) of the invention which comprise administering to a subject a first nucleic acid inhibitor, e.g., dsRNA agent targeting LDHA and a second dsRNA agent targeting HAO1, the first and second nucleic acid inhibitor may be administered to a subject at the same dose or different doses.

The nucleic acid inhibitor can be administered by intravenous infusion over a period of time, on a regular basis. In certain embodiments, after an initial treatment regimen, the treatments can be administered on a less frequent basis.

Administration of a nucleic acid inhibitor can reduce LDHA levels, e.g., in a cell, tissue, blood, urine or other compartment of the patient by at least about 5%, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 39, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or at least about 99% or more. In one embodiment, administration of the nucleic acid inhibitor can reduce LDHA levels, e.g., in a cell, tissue, blood, urine or other compartment of the patient by at least 20%.

Administration of a nucleic acid inhibitor can reduce HAO1 levels, e.g., in a cell, tissue, blood, urine or other compartment of the patient by at least about 5%, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 39, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or at least about 99% or more. In one embodiment, administration of the nucleic acid inhibitor can reduce HAO1 levels, e.g., in a cell, tissue, blood, urine or other compartment of the patient by at least 20%.

Administration of a nucleic acid inhibitor can reduce PRODH2 levels, e.g., in a cell, tissue, blood, urine or other compartment of the patient by at least about 5%, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 39, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or at least about 99% or more. In one embodiment, administration of the nucleic acid inhibitor can reduce PRODH2 levels, e.g., in a cell, tissue, blood, urine or other compartment of the patient by at least 20%.

In the methods (and uses) of the invention which comprise administering to a subject a first nucleic acid inhibitor, e.g., a dsRNA agent targeting LDHA and a second nucleic acid inhibitor, e.g., a dsRNA agent targeting HAO1, the level of inhibition of LDHA may be the same or different that the level of inhibition of HAO1.

In the methods (and uses) of the invention which comprise administering to a subject a dual targeting RNAi agent, the dual targeting RNAi agent may inhibit expression of the LDHA gene and the HAO1 gene to a level substantially the same as the level of inhibition of expression obtained by the contacting of a cell with both dsRNA agents individually, or the dual targeting RNAi agent may inhibit expression of the LDHA gene and the HAO1 gene to a level higher than the level of inhibition of expression obtained by the contacting of a cell with both dsRNA agents individually.

Before administration of a full dose of the nucleic acid inhibitor, patients can be administered a smaller dose, such as a 5% infusion reaction, and monitored for adverse effects, such as an allergic reaction. In another example, the patient can be monitored for unwanted immunostimulatory effects, such as increased cytokine (e.g., TNF-alpha or INF-alpha) levels.

Alternatively, the nucleic acid inhibitor can be administered subcutaneously, i.e., by subcutaneous injection. One or more injections may be used to deliver the desired dose of nucleic acid inhibitor to a subject. The injections may be repeated over a period of time. The administration may be repeated on a regular basis. In certain embodiments, after an initial treatment regimen, the treatments can be administered on a less frequent basis. A repeat-dose regimine may include administration of a therapeutic amount of nucleic acid inhibitor on a regular basis, such as every other day, on a monthly basis, or once a year. In certain embodiments, the nucleic acid inhibitor is administered about once per month to about once per quarter (i.e., about once every three months).

In one embodiment, the method includes administering a composition featured herein such that expression of the target LDHA gene, the target PRODH2 gene and/or the target HAO1 gene is decreased, such as for about 1, 2, 3, 4, 5, 6, 7, 8, 12, 16, 18, 24 hours, 28, 32, or about 36 hours. In one embodiment, expression of the target LDHA gene, the target PRODH2 gene and/or the HAO1 gene is decreased for an extended duration, e.g., at least about two, three, four days or more, e.g., about one week, two weeks, three weeks, or four weeks or longer.

In some embodiments, the nucleic acid inhibitors useful for the methods and compositions featured herein specifically target RNAs (primary or processed) of the target LDHA, PRODH2 and/or HAO1 genes. Compositions and methods for inhibiting the expression of these genes using iRNAs can be prepared and performed as described herein.

Administration of the nucleic acid inhibitors according to the methods of the invention may result in a reduction of the severity, signs, symptoms, and/or markers of such diseases or disorders in a patient with a kidney stone disease. By “reduction” in this context is meant a statistically significant decrease in such level. The reduction can be, for example, at least about 5%, 10%, 15%, 20%, 25%, 30%, 35%, 40%, 45%, 50%, 55%, 60%, 65%, 70%, 75%, 80%, 85%, 90%, 95%, or about 100%.

Efficacy of treatment or prevention of kidney stone disease can be assessed, for example by measuring disease progression, disease remission, symptom severity, reduction in pain, quality of life, dose of a medication required to sustain a treatment effect, level of a disease marker or any other measurable parameter appropriate for a given disease being treated or targeted for prevention. It is well within the ability of one skilled in the art to monitor efficacy of treatment or prevention by measuring any one of such parameters, or any combination of parameters. Comparisons of the later readings with the initial readings provide a physician an indication of whether the treatment is effective. It is well within the ability of one skilled in the art to monitor efficacy of treatment or prevention by measuring any one of such parameters, or any combination of parameters. In connection with the administration of a nucleic acid inhibitor or pharmaceutical composition thereof, “effective against” indicates that administration in a clinically appropriate manner results in a beneficial effect for at least a statistically significant fraction of patients, such as a improvement of symptoms, a cure, a reduction in disease, extension of life, improvement in quality of life, or other effect generally recognized as positive by medical doctors familiar with treating a kidney stone disease and the related causes.

A treatment or preventive effect is evident when there is a statistically significant improvement in one or more parameters of disease status, or by a failure to worsen or to develop symptoms where they would otherwise be anticipated. As an example, a favorable change of at least 10% in a measurable parameter of disease, and such as, at least 20%, 30%, 40%, 50% or more can be indicative of effective treatment. Efficacy for a given nucleic acid inhibitor or formulation of that nucleic acid inhibitor can also be judged using an experimental animal model for the given disease as known in the art, such as alanine-glyoxylate amino transferase deficient (Agxt knockout) mice (see, e.g., Salido, et al. (2006) Proc Natl Acad Sci USA 103:18249) and/or glyoxylate reductase/hydroxypyruvate reductase deficient (Grhpr knockout) mice (see, e.g., Knight, et al. (2011) Am J Physiol Renal Physiol 302:F688).

The invention further provides methods for the use of a nucleic acid inhibitor or a pharmaceutical composition of the invention, e.g., for treating a subject having or at risk of developing a non-primary hyperoxaluria disease that would benefit from reduction in oxalate, in combination with other pharmaceuticals and/or other therapeutic methods, e.g., with known pharmaceuticals and/or known therapeutic methods, such as, for example, those which are currently employed for treating these disorders. For example, in certain embodiments, a nucleic acid inhibitor or pharmaceutical composition of the invention is administered in combination with, e.g., pyridoxine, an ACE inhibitor (angiotensin converting enzyme inhibitors), e.g., benazepril (Lotensin); an angiotensin II receptor antagonist (ARB) (e.g., losartan potassium, such as Merck & Co.'s Cozaar®), e.g., Candesartan (Atacand); an HMG-CoA reductase inhibitor (e.g., a statin); dietary oxalate degrading compounds, e.g., Oxalate decarboxylase (Oxazyme); calcium binding agents, e.g., Sodium cellulose phosphate (Calcibind); diuretics, e.g., thiazide diuretics, such as hydrochlorothiazide (Microzide); phosphate binders, e.g., Sevelamer (Renagel); magnesium and Vitamin B6 supplements; potassium citrate; orthophosphates, bisphosphonates; oral phosphate and citrate solutions; high fluid intake, urinary tract endoscopy; extracorporeal shock wave lithotripsy; kidney dialysis; kidney stone removal (e.g., surgery); and kidney/liver transplant; or a combination of any of the foregoing.

### III. Nucleic Acid Inhibitors for Use in the Methods of the Invention

### A. Double Stranded Ribonucleic Acid Agents of the Invention

In one embodiment, a nucleic acid inhibitor for use in the methods of the invention is a dsRNA agent. In one embodiment, the dsRNA agent targets an LDHA gene. In one embodiment, the dsRNA agent targets a PRODH2 gene. In another embodiment, the dsRNA agent targets an HAO1 gene. In one embodiment, the dsRNA agent is a dual targeting dsRNA agent targeting an LDHA gene and an HAO1 gene.

Suitable dsRNA agents for use in the methods of the invention are known in the art and described in, for example, U.S. Patent Publication No. 20200113927 (Alnylam Pharmaceuticals, Inc.); U.S. Patent Publication Nos. 2017/0304446 (Lumasiran) (Alnylam Pharmaceuticals, Inc.), 2017/0306332 (Dicerna Pharmaceuticals), and 2019/0323014 (Dicerna Pharmaceuticals); U.S. Pat. No. 10,478,500 (Lumasiran) (Alnylam Pharmaceuticals, Inc.) and 10,351,854 (Dicerna Pharmaceuticals); and PCT Publication Nos. WO 2019/014530 (Attorney Docket No.: 121301-07520) and WO 2019/075419 (Dicerna Pharmaceuticals), the entire contents of each of which are incorporated herein by reference. Any of these agents may further comprise a ligand. In one embodiment, a suitable dsRNA agent is nedosiran (formerly referred to as DCR-PHXC) (Dicerna Pharmaceuticals).

In certain specific embodiments, a nucleic acid inhibitor of the present invention is a dsRNA agent which inhibits the expression of an LDHA gene and is selected from the group of agents listed in any one of Tables 2-3. In other embodiments, a nucleic acid inhibitor of the present invention is a dsRNA agent which inhibits the expression of an HAO1 gene and is selected from the group of agents listed in any one of Tables 4-12. In other embodiments, a nucleic acid inhibitor of the present invention is a dsRNA agent which inhibits the expression of a PRODH2 gene and is selected from the group of agents listed in any one of Tables 15-16. In yet other embodiments, nucleic acid inhibitor of the present invention is an dual targeting iRNA agent that inhibits the expression of an LDHA gene and an HAO1 gene, wherein the first dsRNA inhibits expression of an LDHA gene and is selected from the group of agents listed in any one of Tables 2-3, and the first dsRNA inhibits expression of an HAO1 gene and is selected from the group of agents listed in any one of Tables 4-12.

The dsRNAs of the invention targeting LDHA may include an RNA strand (the antisense strand) having a region which is about 30 nucleotides or less in length, e.g., 15-30, 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 nucleotides in length, which region is substantially complementary to at least part of an mRNA transcript of an LDHA gene.

The dsRNAs of the invention targeting HAO1 may include an RNA strand (the antisense strand) having a region which is about 30 nucleotides or less in length, e.g., 15-30, 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 nucleotides in length, which region is substantially complementary to at least part of an mRNA transcript of an HAO1 gene.

The dsRNAs of the invention targeting PRODH2 may include an RNA strand (the antisense strand) having a region which is about 30 nucleotides or less in length, e.g., 15-30, 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 nucleotides in length, which region is substantially complementary to at least part of an mRNA transcript of a PRODH2 gene.

When the dsRNA agent is a dual targeting agent, as described herein, the agent targeting LDHA may include an antisense strand comprising a region of complementarity to LDHA which is the same length or a different length from the region of complementarity of the antisense strand of the agent targeting HAO1.

In some embodiments, one or both of the strands of the double stranded RNAi agents of the invention is up to 66 nucleotides in length, e.g., 36-66, 26-36, 25-36, 31-60, 22-43, 27-53 nucleotides in length, with a region of at least 19 contiguous nucleotides that is substantially complementary to at least a part of an mRNA transcript of an LDHA gene. In some embodiments, such dsRNA agents having longer length antisense strands may include a second RNA strand (the sense strand) of 20-60 nucleotides in length wherein the sense and antisense strands form a duplex of 18-30 contiguous nucleotides.

In other embodiments, one or both of the strands of the double stranded RNAi agents of the invention is up to 66 nucleotides in length, e.g., 36-66, 26-36, 25-36, 31-60, 22-43, 27-53 nucleotides in length, with a region of at least 19 contiguous nucleotides that is substantially complementary to at least a part of an mRNA transcript of an HAO1 gene. In some embodiments, such dsRNA agents having longer length antisense strands may include a second RNA strand (the sense strand) of 20-60 nucleotides in length wherein the sense and antisense strands form a duplex of 18-30 contiguous nucleotides.

In embodiments in which a first dsRNA agent targeting LDHA and a second dsRNA agent targeting HAO1 are covalently attached, the duplex lengths of the first agent and the second agent may be the same or different.

The use of these dsRNA agents described herein enables the targeted degradation of mRNAs of an LDHA gene, a PRODH2 gene and/or an HAO1 gene in mammals.

The dsRNA includes an antisense strand having a region of complementarity which is complementary to at least a part of an mRNA formed in the expression of an LDHA gene or an HAO1 gene or a PRODH2 gene. The region of complementarity is about 30 nucleotides or less in length (e.g., about 30, 29, 28, 27, 26, 25, 24, 23, 22, 21, 20, 19, or 18 nucleotides or less in length). Upon contact with a cell expressing the target gene, the iRNA inhibits the expression of the target gene (e.g., a human, a primate, a non-primate, or a bird target gene) by at least about 10% as assayed by, for example, a PCR or branched DNA (bDNA)-based method, or by a protein-based method, such as by immunofluorescence analysis, using, for example, Western Blotting or flowcytometric techniques.

A dsRNA includes two RNA strands that are complementary and hybridize to form a duplex structure under conditions in which the dsRNA will be used. One strand of a dsRNA (the antisense strand) includes a region of complementarity that is substantially complementary, and generally fully complementary, to a target sequence. The target sequence can be derived from the sequence of an mRNA formed during the expression of an LDHA gene or an HAO1 gene or a PRODH2 gene. The other strand (the sense strand) includes a region that is complementary to the antisense strand, such that the two strands hybridize and form a duplex structure when combined under suitable conditions. As described elsewhere herein and as known in the art, the complementary sequences of a dsRNA can also be contained as self-complementary regions of a single nucleic acid molecule, as opposed to being on separate oligonucleotides.

Generally, the duplex structure is between 15 and 30 base pairs in length, e.g., between, 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 base pairs in length. Ranges and lengths intermediate to the above recited ranges and lengths are also contemplated to be part of the invention.

Similarly, the region of complementarity to the target sequence is between 15 and 30 nucleotides in length, e.g., between 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 nucleotides in length. Ranges and lengths intermediate to the above recited ranges and lengths are also contemplated to be part of the invention.

In some embodiments, the dsRNA is between about 15 and about 23 nucleotides in length, or between about 25 and about 30 nucleotides in length. In general, the dsRNA is long enough to serve as a substrate for the Dicer enzyme. For example, it is well known in the art that dsRNAs longer than about 21-23 nucleotides can serve as substrates for Dicer. As the ordinarily skilled person will also recognize, the region of an RNA targeted for cleavage will most often be part of a larger RNA molecule, often an mRNA molecule. Where relevant, a “part” of an mRNA target is a contiguous sequence of an mRNA target of sufficient length to allow it to be a substrate for RNAi-directed cleavage (i.e., cleavage through a RISC pathway).

One of skill in the art will also recognize that the duplex region is a primary functional portion of a dsRNA, e.g., a duplex region of about 9 to 36 base pairs, e.g., about 10-36, 11-36, 12-36, 13-36, 14-36, 15-36, 9-35, 10-35, 11-35, 12-35, 13-35, 14-35, 15-35, 9-34, 10-34, 11-34, 12-34, 13-34, 14-34, 15-34, 9-33, 10-33, 11-33, 12-33, 13-33, 14-33, 15-33, 9-32, 10-32, 11-32, 12-32, 13-32, 14-32, 15-32, 9-31, 10-31, 11-31, 12-31, 13-32, 14-31, 15-31, 15-30, 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 base pairs. Thus, in one embodiment, to the extent that it becomes processed to a functional duplex, of e.g., 15-30 base pairs, that targets a desired RNA for cleavage, an RNA molecule or complex of RNA molecules having a duplex region greater than 30 base pairs is a dsRNA. Thus, an ordinarily skilled artisan will recognize that in one embodiment, a miRNA is a dsRNA. In another embodiment, a dsRNA is not a naturally occurring miRNA. In another embodiment, an iRNA agent useful to target LDHA or HAO1 or PRODH2 expression or LDHA and HAO1 expression is not generated in the target cell by cleavage of a larger dsRNA.

A dsRNA as described herein can further include one or more single-stranded nucleotide overhangs e.g., 1, 2, 3, or 4 nucleotides. dsRNAs having at least one nucleotide overhang can have unexpectedly superior inhibitory properties relative to their blunt-ended counterparts. A nucleotide overhang can comprise or consist of a nucleotide/nucleoside analog, including a deoxynucleotide/nucleoside. The overhang(s) can be on the sense strand, the antisense strand or any combination thereof. Furthermore, the nucleotide(s) of an overhang can be present on the 5-end, 3′-end or both ends of either an antisense or sense strand of a dsRNA.

A dsRNA can be synthesized by standard methods known in the art as further discussed below, e.g., by use of an automated DNA synthesizer, such as are commercially available from, for example, Biosearch, Applied Biosystems, Inc.

A dsRNA of the invention may be prepared using a two-step procedure. First, the individual strands of the double-stranded RNA molecule are prepared separately. Then, the component strands are annealed. The individual strands of the siRNA compound can be prepared using solution-phase or solid-phase organic synthesis or both. Organic synthesis offers the advantage that the oligonucleotide strands comprising unnatural or modified nucleotides can be easily prepared. Single-stranded oligonucleotides of the invention can be prepared using solution-phase or solid-phase organic synthesis or both.

In one aspect, a dsRNA of the invention targets an LDHA gene and includes at least two nucleotide sequences, a sense sequence and an anti-sense sequence. The sense strand sequence is selected from the group of sequences provided in any one of Tables 2-3 and the corresponding nucleotide sequence of the antisense strand is selected from the group of sequences of any one of Tables 2-3. In this aspect, one of the two sequences is complementary to the other of the two sequences, with one of the sequences being substantially complementary to a sequence of an mRNA generated in the expression of an LDHA gene. As such, in this aspect, a dsRNA will include two oligonucleotides, where one oligonucleotide is described as the sense strand (passenger strand) in any one of Tables 2-3 and the second oligonucleotide is described as the corresponding antisense strand (guide strand) of the sense strand in any one of Tables 2-3. In one embodiment, the substantially complementary sequences of the dsRNA are contained on separate oligonucleotides. In another embodiment, the substantially complementary sequences of the dsRNA are contained on a single oligonucleotide.

In another aspect, a dsRNA of the invention targets an HAO1 gene and includes at least two nucleotide sequences, a sense sequence and an anti-sense sequence. The sense strand sequence is selected from the group of sequences provided in any one of Tables 4-14 and the corresponding nucleotide sequence of the antisense strand of the sense strand is selected from the group of sequences of any one of Tables 4-14. In this aspect, one of the two sequences is complementary to the other of the two sequences, with one of the sequences being substantially complementary to a sequence of an mRNA generated in the expression of an HAO1 gene. As such, in this aspect, a dsRNA will include two oligonucleotides, where one oligonucleotide is described as the sense strand (passenger strand) in any one of Tables 4-14 and the second oligonucleotide is described as the corresponding antisense strand (guide strand) of the sense strand in any one of Tables 4-14. In one embodiment, the substantially complementary sequences of the dsRNA are contained on separate oligonucleotides. In another embodiment, the substantially complementary sequences of the dsRNA are contained on a single oligonucleotide.

In yet another aspect, a dsRNA of the invention targets a PRODH2 gene and includes at least two nucleotide sequences, a sense sequence and an anti-sense sequence. The sense strand sequence is selected from the group of sequences provided in any one of Tables 15-16 and the corresponding nucleotide sequence of the antisense strand of the sense strand is selected from the group of sequences of any one of Tables 15-16. In this aspect, one of the two sequences is complementary to the other of the two sequences, with one of the sequences being substantially complementary to a sequence of an mRNA generated in the expression of a PRODH2 gene. As such, in this aspect, a dsRNA will include two oligonucleotides, where one oligonucleotide is described as the sense strand (passenger strand) in any one of Tables 15-16 and the second oligonucleotide is described as the corresponding antisense strand (guide strand) of the sense strand in any one of Tables 15-16. In one embodiment, the substantially complementary sequences of the dsRNA are contained on separate oligonucleotides. In another embodiment, the substantially complementary sequences of the dsRNA are contained on a single oligonucleotide.

It will be understood that, although the sequences in Tables 2-16 are described as modified, unmodified, unconjugated. and/or conjugated sequences, the RNA of the dsRNA of the invention e.g., a dsRNA of the invention, may comprise any one of the sequences set forth in any one of Table 2-16 that is un-modified, un-conjugated, and/or modified and/or conjugated differently than described therein.

The skilled person is well aware that dsRNAs having a duplex structure of between about 20 and 23 base pairs, e.g., 21, base pairs have been hailed as particularly effective in inducing RNA interference (Elbashir et al., (2001) EMBO J., 20:6877-6888). However, others have found that shorter or longer RNA duplex structures can also be effective (Chu and Rana (2007) RNA 14:1714-1719; Kim et al. (2005) Nat Biotech 23:222-226). In the embodiments described above, by virtue of the nature of the oligonucleotide sequences provided herein, dsRNAs described herein can include at least one strand of a length of minimally 21 nucleotides. It can be reasonably expected that shorter duplexes minus only a few nucleotides on one or both ends can be similarly effective as compared to the dsRNAs described above. Hence, dsRNAs having a sequence of at least 15, 16, 17, 18, 19, 20, or more contiguous nucleotides derived from one of the sequences provided herein, and differing in their ability to inhibit the expression of an LDHA gene or an HAO1 gene or a PRODH2 gene by not more than about 5, 10, 15, 20, 25, or 30% inhibition from a dsRNA comprising the full sequence, are contemplated to be within the scope of the present invention.

In addition, the RNAs described in any one of Tables 2-3 identify a site(s) in an LDHA transcript that is susceptible to RISC-mediated cleavage, the RNAs described in any one of Tables 4-14 identify a site(s) in an HAO1 transcript that is susceptible to RISC-mediated cleavage, and those RNAs described in any one of Tables 15-16 identify a site(s) in a PRODH2 transcript that is susceptible to RISC-mediated cleavage. As such, the present invention further features iRNAs that target within this site(s). As used herein, an iRNA is said to target within a particular site of an RNA transcript if the iRNA promotes cleavage of the transcript anywhere within that particular site. Such an iRNA will generally include at least about 15 contiguous nucleotides from one of the sequences provided herein coupled to additional nucleotide sequences taken from the region contiguous to the selected sequence in the gene.

While a target sequence is generally about 15-30 nucleotides in length, there is wide variation in the suitability of particular sequences in this range for directing cleavage of any given target RNA. Various software packages and the guidelines set out herein provide guidance for the identification of optimal target sequences for any given gene target, but an empirical approach can also be taken in which a “window” or “mask” of a given size (as a non-limiting example, 21 nucleotides) is literally or figuratively (including, e.g., in silico) placed on the target RNA sequence to identify sequences in the size range that can serve as target sequences. By moving the sequence “window” progressively one nucleotide upstream or downstream of an initial target sequence location, the next potential target sequence can be identified, until the complete set of possible sequences is identified for any given target size selected. This process, coupled with systematic synthesis and testing of the identified sequences (using assays as described herein or as known in the art) to identify those sequences that perform optimally can identify those RNA sequences that, when targeted with an iRNA agent, mediate the best inhibition of target gene expression. Thus, while the sequences identified herein represent effective target sequences, it is contemplated that further optimization of inhibition efficiency can be achieved by progressively “walking the window” one nucleotide upstream or downstream of the given sequences to identify sequences with equal or better inhibition characteristics.

Further, it is contemplated that for any sequence identified herein, further optimization could be achieved by systematically either adding or removing nucleotides to generate longer or shorter sequences and testing those sequences generated by walking a window of the longer or shorter size up or down the target RNA from that point. Again, coupling this approach to generating new candidate targets with testing for effectiveness of iRNAs based on those target sequences in an inhibition assay as known in the art and/or as described herein can lead to further improvements in the efficiency of inhibition. Further still, such optimized sequences can be adjusted by, e.g., the introduction of modified nucleotides as described herein or as known in the art, addition or changes in overhang, or other modifications as known in the art and/or discussed herein to further optimize the molecule (e.g., increasing serum stability or circulating half-life, increasing thermal stability, enhancing transmembrane delivery, targeting to a particular location or cell type, increasing interaction with silencing pathway enzymes, increasing release from endosomes) as an expression inhibitor.

A dsRNA agent as described herein can contain one or more mismatches to the target sequence. In one embodiment, an iRNA as described herein contains no more than 3 mismatches. If the antisense strand of the iRNA contains mismatches to a target sequence, it is preferable that the area of mismatch is not located in the center of the region of complementarity. If the antisense strand of the iRNA contains mismatches to the target sequence, it is preferable that the mismatch be restricted to be within the last 5 nucleotides from either the 5′- or 3′-end of the region of complementarity. For example, for a 23 nucleotide iRNA agent the strand which is complementary to a region of an LDHA gene or an HAO1 gene or a PRODH2 gene, generally does not contain any mismatch within the central 13 nucleotides. The methods described herein or methods known in the art can be used to determine whether an iRNA containing a mismatch to a target sequence is effective in inhibiting the expression of an LDHA gene, a PRODH2 gene and/or an HAO1 gene. Consideration of the efficacy of iRNAs with mismatches in inhibiting expression of an LDHA gene, a PRODH2 gene and/or an HAO1 gene is important, especially if the particular region of complementarity in an LDHA gene, a PRODH2 gene and/or HAO1 gene is known to have polymorphic sequence variation within the population.

The dual targeting RNAi agents of the invention, which include two dsRNA agents, are covalently attached via, e.g., a covalent linker. Covalent linkers are well known in the art and include, e.g., nucleic acid linkers, peptide linkers, carbohydrate linkers, and the like. The covalent linker can include RNA and/or DNA and/or a peptide. The linker can be single stranded, double stranded, partially single strands, or partially double stranded. Modified nucleotides or a mixture of nucleotides can also be present in a nucleic acid linker.

Suitable linkers for use in the dual targeting agent of the invention include those described in U.S. Pat. No. 9,187,746, the entire contents of which are incorporated herein by reference.

In some embodiments the linker includes a disulfide bond. The linker can be cleavable or non-cleavable.

The linker can be, e.g., dTsdTuu=(5′-2′deoxythymidyl-3′-thiophosphate-5′-2′deoxythymidyl-3′-phosphate-5′-uridyl-3′-phosphate-5′-uridyl-3′-phosphate); rUsrU (a thiophosphate linker: 5′-uridyl-3′-thiophosphate-5′-uridyl-3′-phosphate); an rUrU linker; dTsdTaa (aadTsdT, 5′-2′deoxythymidyl-3′-thiophosphate-5′-2′deoxythymidyl-3′-phosphate-5′-adenyl-3′-phosphate-5′-adenyl-3′-phosphate); dTsdT (5′-2′deoxythymidyl-3′-thiophosphate-5′-2′ deoxythymidyl-3′-phosphate); dTsdTuu=uudTsdT=5′-2′deoxythymidyl-3′-thiophosphate-5′-2′deoxythymidyl-3′-phosphate-5′-uridyl-3′-phosphate-5′-uridyl-3′-phosphate.

The linker can be a polyRNA, such as poly(5′-adenyl-3′-phosphate-AAAAAAAA) or poly(5′-cytidyl-3′-phosphate-5′-uridyl-3′-phosphate-CUCUCUCU)), e.g., Xn single stranded poly RNA linker wherein n is an integer from 2-50 inclusive, such as, 4-15 inclusive, or 7-8 inclusive. Modified nucleotides or a mixture of nucleotides can also be present in said polyRNA linker. The covalent linker can be a polyDNA, such as poly(5′-2′deoxythymidyl-3′-phosphate-TTTTTTTT), e.g., wherein n is an integer from 2-50 inclusive, such as 4-15 inclusive, or 7-8 inclusive. Modified nucleotides or a mixture of nucleotides can also be present in said polyDNA linker, a single stranded polyDNA linker wherein n is an integer from 2-50 inclusive, such as 4-15 inclusive, or 7-8 inclusive. Modified nucleotides or a mixture of nucleotides can also be present in said polyDNA linker.

The linker can include a disulfide bond, optionally a bis-hexyl-disulfide linker. In one embodiment, the disulfide linker is

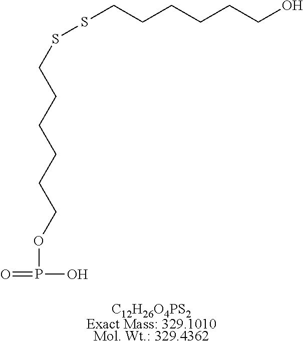

The linker can include a peptide bond, e.g., include amino acids. In one embodiment, the covalent linker is a 1-10 amino acid long linker, such as, comprising 4-5 amino acids, optionally X-Gly-Phe-Gly-Y wherein X and Y represent any amino acid.

The linker can include HEG, a hexaethyleneglycol linker.

The covalent linker can attach the sense strand of the first dsRNA agent to the sense strand of the second dsRNA agent; the antisense strand of the first dsRNA agent to the antisense strand of the second dsRNA agent; the sense strand of the first dsRNA agent to the antisense strand of the second dsRNA agent; or the antisense strand of the first dsRNA agent to the sense strand of the second dsRNA agent.

In some embodiments, the covalent linker further comprises at least one ligand, described below.

i. Modified dsRNA Agent of the Invention

In one embodiment, the nucleic acid, e.g., RNA, of a nucleic acid inhibitor of the invention is un-modified, and does not comprise, e.g., chemical modifications and/or conjugations known in the art and described herein. In another embodiment, the nucleic acid, e.g., RNA, of a nucleic acid inhibitor of the invention is chemically modified to enhance stability or other beneficial characteristics. In certain embodiments of the invention, substantially all of the nucleotides of a nucleic acid inhibitor of the invention are modified. In other embodiments of the invention, all of the nucleotides of a nucleic acid inhibitor of the invention are modified. Nucleic acid inhibitors of the invention in which “substantially all of the nucleotides are modified” are largely but not wholly modified and can include not more than 5, 4, 3, 2, or 1 unmodified nucleotides.

In embodiments in which a first nucleic acid inhibitor, e.g., dsRNA agent targeting LDHA, and a second nucleic acid inhibitor, e.g., dsRNA agent targeting HAO1, are covalently attached (i.e., a dual targeting RNAi agent), substantially all of the nucleotides of the first agent and substantially all of the nucleotides of the second agent may be independently modified; all of the nucleotides of the first agent may be modified and all of the nucleotides of the second agent may be independently modified; substantially all of the nucleotides of the first agent and all of the nucleotides of the second agent may be independently modified; or all of the nucleotides of the first agent may be modified and substantially all of the nucleotides of the second agent may be independently modified.

In some aspects of the invention, substantially all of the nucleotides of a nucleic acid inhibitor of the invention are modified and the nucleic acid inhibitors comprise no more than 10 nucleotides comprising 2′-fluoro modifications (e.g., no more than 9 2′-fluoro modifications, no more than 8 2′-fluoro modifications, no more than 7 2′-fluoro modifications, no more than 6 2′-fluoro modifications, no more than 5 2′-fluoro modifications, no more than 4 2′-fluoro modifications, no more than 5 2′-fluoro modifications, no more than 4 2′-fluoro modifications, no more than 3 2′-fluoro modifications, or no more than 2 2′-fluoro modifications). For example, in some embodiments, the sense strand comprises no more than 4 nucleotides comprising 2′-fluoro modifications (e.g., no more than 3 2′-fluoro modifications, or no more than 2 2′-fluoro modifications). In other embodiments, the antisense strand comprises no more than 6 nucleotides comprising 2′-fluoro modifications (e.g., no more than 5 2′-fluoro modifications, no more than 4 2′-fluoro modifications, no more than 4 2′-fluoro modifications, or no more than 2 2′-fluoro modifications).

In embodiments in which a first nucleic acid inhibitor, e.g., dsRNA agent targeting LDHA, and a second nucleic acid inhibitor, e.g., dsRNA agent targeting HAO1, are covalently attached (i.e., a dual targeting RNAi agent), substantially all of the nucleotides of the first agent and/or substantially all of the nucleotides of the second agent may be independently modified and the first and second agents may independently comprise no more than 10 nucleotides comprising 2′-fluoro modifications.

In other aspects of the invention, all of the nucleotides of a nucleic acid inhibitor of the invention are modified and the nucleic acid inhibitors comprise no more than 10 nucleotides comprising 2′-fluoro modifications (e.g., no more than 9 2′-fluoro modifications, no more than 8 2′-fluoro modifications, no more than 7 2′-fluoro modifications, no more than 6 2′-fluoro modifications, no more than 5 2′-fluoro modifications, no more than 4 2′-fluoro modifications, no more than 5 2′-fluoro modifications, no more than 4 2′-fluoro modifications, no more than 3 2′-fluoro modifications, or no more than 2 2′-fluoro modifications).

In embodiments in which a first nucleic acid inhibitor, e.g., dsRNA agent targeting LDHA, and a second nucleic acid inhibitor, e.g., dsRNA agent targeting HAO1, are covalently attached (i.e., a dual targeting RNAi agent), all of the nucleotides of the first agent and/or all of the nucleotides of the second agent may be independently modified and the first and second agents may independently comprise no more than 10 nucleotides comprising 2′-fluoro modifications.

In one embodiment, a nucleic acid inhibitor of the invention further comprises a 5′-phosphate or a 5′-phosphate mimic at the 5′ nucleotide of the antisense strand. In another embodiment, the double stranded RNAi agent further comprises a 5′-phosphate mimic at the 5′ nucleotide of the antisense strand. In a specific embodiment, the 5′-phosphate mimic is a 5′-vinyl phosphonate (5′-VP).

In embodiments in which a first nucleic acid inhibitor, e.g., dsRNA agent targeting LDHA, and a second nucleic acid inhibitor, e.g., dsRNA agent targeting HAO1, are covalently attached (i.e., a dual targeting RNAi agent), the first agent may further comprise a 5′-phosphate or a 5′-phosphate mimic at the 5′ nucleotide of the antisense strand; the second agent may further comprise a 5′-phosphate or a 5′-phosphate mimic at the 5′ nucleotide of the antisense strand; or the first agent and the second agent may further independently comprise a 5′-phosphate or a 5′-phosphate mimic at the 5′ nucleotide of the antisense strand.

The nucleic acids featured in the invention can be synthesized and/or modified by methods well established in the art, such as those described in “Current protocols in nucleic acid chemistry,” Beaucage, S. L. et al. (Edrs.), John Wiley & Sons, Inc., New York, NY, USA, which is hereby incorporated herein by reference. Modifications include, for example, end modifications, e.g., 5′-end modifications (phosphorylation, conjugation, inverted linkages) or 3′-end modifications (conjugation, DNA nucleotides, inverted linkages, etc.); base modifications, e.g., replacement with stabilizing bases, destabilizing bases, or bases that base pair with an expanded repertoire of partners, removal of bases (abasic nucleotides), or conjugated bases; sugar modifications (e.g., at the 2′-position or 4′-position) or replacement of the sugar; and/or backbone modifications, including modification or replacement of the phosphodiester linkages. Specific examples of nucleic acid inhibitor compounds useful in the embodiments described herein include, but are not limited to nucleic acid inhibitors containing modified backbones or no natural internucleoside linkages. Nucleic acid inhibitors having modified backbones include, among others, those that do not have a phosphorus atom in the backbone. For the purposes of this specification, and as sometimes referenced in the art, modified nucleic acid inhibitors that do not have a phosphorus atom in their internucleoside backbone can also be considered to be oligonucleosides. In some embodiments, a modified nucleic acid inhibitor will have a phosphorus atom in its internucleoside backbone.

Modified nucleic acid inhibitor backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′-linked analogs of these, and those having inverted polarity wherein the adjacent pairs of nucleoside units are linked 3′-5′ to 5′-3′ or 2′-5′ to 5′-2′. Various salts, mixed salts and free acid forms are also included.

Representative U.S. patents that teach the preparation of the above phosphorus-containing linkages include, but are not limited to, U.S. Pat. Nos. 3,687,808; 4,469,863; 4,476,301; 5,023,243; 5,177,195; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,316; 5,550,111; 5,563,253; 5,571,799; 5,587,361; 5,625,050; 6,028,188; 6,124,445; 6,160,109; 6,169,170; 6,172,209; 6,239,265; 6,277,603; 6,326,199; 6,346,614; 6,444,423; 6,531,590; 6,534,639; 6,608,035; 6,683,167; 6,858,715; 6,867,294; 6,878,805; 7,015,315; 7,041,816; 7,273,933; 7,321,029; and U.S. Pat. RE39464, the entire contents of each of which are hereby incorporated herein by reference.

Modified nucleic acid inhibitor backbones that do not include a phosphorus atom therein have backbones that are formed by short chain alkyl or cycloalkyl internucleoside linkages, mixed heteroatoms and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Representative U.S. patents that teach the preparation of the above oligonucleosides include, but are not limited to, U.S. Pat. Nos. 5,034,506; 5,166,315; 5,185,444; 5,214,134; 5,216,141; 5,235,033; 5,64,562; 5,264,564; 5,405,938; 5,434,257; 5,466,677; 5,470,967; 5,489,677; 5,541,307; 5,561,225; 5,596,086; 5,602,240; 5,608,046; 5,610,289; 5,618,704; 5,623,070; 5,663,312; 5,633,360; 5,677,437; and, 5,677,439, the entire contents of each of which are hereby incorporated herein by reference.

In other embodiments, suitable RNA mimetics are contemplated for use in nucleic acid inhibitors, in which both the sugar and the internucleoside linkage, i.e., the backbone, of the nucleotide units are replaced with novel groups. The base units are maintained for hybridization with an appropriate nucleic acid target compound. One such oligomeric compound, an RNA mimetic that has been shown to have excellent hybridization properties, is referred to as a peptide nucleic acid (PNA). In PNA compounds, the sugar backbone of an RNA is replaced with an amide containing backbone, in particular an aminoethylglycine backbone. The nucleobases are retained and are bound directly or indirectly to aza nitrogen atoms of the amide portion of the backbone. Representative U.S. patents that teach the preparation of PNA compounds include, but are not limited to, U.S. Pat. Nos. 5,539,082; 5,714,331; and 5,719,262, the entire contents of each of which are hereby incorporated herein by reference. Additional PNA compounds suitable for use in the iRNAs of the invention are described in, for example, in Nielsen et al., Science, 1991, 254, 1497-1500.

Some embodiments featured in the invention include nucleic acid inhibitors, e.g., RNAs, with phosphorothioate backbones and oligonucleosides with heteroatom backbones, and in particular —CH₂—NH—CH₂—, —CH₂—N(CH₃)—O—CH₂--[known as a methylene (methylimino) or MMI backbone], —CH₂—O—N(CH₃)—CH₂—, —CH₂—N(CH₃)—N(CH₃)—CH₂— and —N(CH₃)—CH₂—CH₂— of the above-referenced U.S. Pat. No. 5,489,677, and the amide backbones of the above-referenced U.S. Pat. No. 5,602,240. In some embodiments, the RNAs featured herein have morpholino backbone structures of the above-referenced U.S. Pat. No. 5,034,506. The native phosphodiester backbone can be represented as O—P(O)(OH)—OCH2-.

Modified nucleic acid inhibitors can also contain one or more substituted sugar moieties. The nucleic acid inhibitors, e.g., dsRNAs, featured herein can include one of the following at the 2′-position: OH; F; O-, S-, or N-alkyl; O-, S-, or N-alkenyl; O-, S- or N-alkynyl; or O-alkyl-O-alkyl, wherein the alkyl, alkenyl and alkynyl can be substituted or unsubstituted C₁ to C₁₀ alkyl or C₂ to C₁₀ alkenyl and alkynyl. Exemplary suitable modifications include O[(CH₂)ₙO]ₘCH₃, O(CH₂)·ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. In other embodiments, dsRNAs include one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an iRNA, or a group for improving the pharmacodynamic properties of a nucleic acid inhibitor, and other substituents having similar properties. In some embodiments, the modification includes a 2′-methoxyethoxy (2′-O—CH₂CH₂OCH₃, also known as 2′-O-(2-methoxyethyl) or 2′-MOE) (Martin et al., Helv. Chim. Acta, 1995, 78:486-504) i.e., an alkoxy-alkoxy group. Another exemplary modification is 2′-dimethylaminooxyethoxy, i.e., a O(CH₂)₂ON(CH₃)₂ group, also known as 2′-DMAOE, as described in examples herein below, and 2′-dimethylaminoethoxyethoxy (also known in the art as 2′-O-dimethylaminoethoxyethyl or 2′-DMAEOE), i.e., 2′-O—CH₂—O—CH₂—N(CH₃)₂. Further exemplary modifications include: 5′-Me-2′-F nucleotides, 5′-Me-2′-OMe nucleotides, 5′-Me-2′-deoxynucleotides, (both R and S isomers in these three families); 2′-alkoxyalkyl; and 2′-NMA (N-methylacetamide).

Other modifications include 2′-methoxy (2′-OCH₃), 2′-aminopropoxy (2′-OCH₂CH₂CH₂NH₂) and 2′-fluoro (2′-F). Similar modifications can also be made at other positions on the RNA of a nucleic acid inhibitor, particularly the 3′ position of the sugar on the 3′ terminal nucleotide or in 2′-5′ linked dsRNAs and the 5′ position of 5′ terminal nucleotide. Nucleic acid inhibitors can also have sugar mimetics such as cyclobutyl moieties in place of the pentofuranosyl sugar. Representative U.S. patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, certain of which are commonly owned with the instant application. The entire contents of each of the foregoing are hereby incorporated herein by reference.

Additional nucleotides having modified or substituted sugar moieties for use in the nucleic acid inhibitors of the invention include nucleotides comprising a bicyclic sugar. A “bicyclic sugar” is a furanosyl ring modified by the bridging of two atoms. A“bicyclic nucleoside” (“BNA”) is a nucleoside having a sugar moiety comprising a bridge connecting two carbon atoms of the sugar ring, thereby forming a bicyclic ring system. In certain embodiments, the bridge connects the 4′-carbon and the 2′-carbon of the sugar ring. Thus, in some embodiments a nucleic acid inhibitor may include one or more locked nucleic acids. A “locked nucleic acid” (“LNA”) is a nucleotide having a modified ribose moiety in which the ribose moiety comprises an extra bridge connecting the 2′ and 4′ carbons. In other words, an LNA is a nucleotide comprising a bicyclic sugar moiety comprising a 4′-CH₂—O-2′ bridge. This structure effectively “locks” the ribose in the 3′-endo structural conformation. The addition of locked nucleic acids to polynucleotide agents has been shown to increase polynucleotide agent stability in serum, and to reduce off-target effects (Elmen, J. et al., (2005) Nucleic Acids Research 33(1):439-447; Mook, O R. et al., (2007) Mol Canc Ther 6(3):833-843; Grunweller, A. et al., (2003) Nucleic Acids Research 31(12):3185-3193).

Examples of bicyclic nucleosides for use in the nucleic acid inhibitors of the invention include without limitation nucleosides comprising a bridge between the 4′ and the 2′ ribosyl ring atoms. In certain embodiments, the nucleic acid inhibitors of the invention include one or more bicyclic nucleosides comprising a 4′ to 2′ bridge. Examples of such 4′ to 2′ bridged bicyclic nucleosides, include but are not limited to 4′-(CH2)-O-2′ (LNA); 4′-(CH2)-S-2′; 4′-(CH2)2-O-2′ (ENA); 4′-CH(CH3)-O-2′ (also referred to as “constrained ethyl” or “cEt”) and 4′-CH(CH2OCH3)-O-2′ (and analogs thereof; see, e.g., U.S. Pat. No. 7,399,845); 4′-C(CH3)(CH3)-O-2′ (and analogs thereof; see e.g., U.S. Pat. No. 8,278,283); 4′-CH2-N(OCH3)-2′ (and analogs thereof; see e.g., U.S. Pat. No. 8,278,425); 4′-CH2-O—N(CH3)-2′ (see, e.g., U.S. Patent Publication No. 2004/0171570); 4′-CH2-N(R)—O-2′, wherein R is H, C1-C12 alkyl, or a protecting group (see, e.g., U.S. Pat. No. 7,427,672); 4′-CH2-C(H)(CH3)-2′ (see, e.g., Chattopadhyaya et al., J. Org. Chem., 2009, 74, 118-134); and 4′-CH2-C(═CH2)-2′ (and analogs thereof; see, e.g., U.S. Pat. No. 8,278,426). The entire contents of each of the foregoing are hereby incorporated herein by reference.

Additional representative U.S. patents and US Patent Publications that teach the preparation of locked nucleic acid nucleotides include, but are not limited to, the following: U.S. Pat. Nos. 6,268,490; 6,525,191; 6,670,461; 6,770,748; 6,794,499; 6,998,484; 7,053,207; 7,034,133; 7,084,125; 7,399,845; 7,427,672; 7,569,686; 7,741,457; 8,022,193; 8,030,467; 8,278,425; 8,278,426; 8,278,283; US 2008/0039618; and US 2009/0012281, the entire contents of each of which are hereby incorporated herein by reference.

Any of the foregoing bicyclic nucleosides can be prepared having one or more stereochemical sugar configurations including for example α-L-ribofuranose and β-D-ribofuranose (see WO 99/14226).

In one particular embodiment of the invention, a nucleic acid inhibitor can include one or more constrained ethyl nucleotides. As used herein, a “constrained ethyl nucleotide” or “cEt” is a locked nucleic acid comprising a bicyclic sugar moiety comprising a 4′-CH(CH₃)—O-2′ bridge. In one embodiment, a constrained ethyl nucleotide is in an S conformation and is referred to as an “S-constrained ethyl nucleotide” or “S-cEt.”

Modified nucleotides included in the nucleic acid inhibitors of the invention can also contain one or more sugar mimetics. For example, the nucleic acid inhibitor may include a “modified tetrahydropyran nucleotide” or “modified THP nucleotide.” A “modified tetrahydropyran nucleotide” has a six-membered tetrahydropyran “sugar” substituted in for the pentofuranosyl residue in normal nucleotides (a sugar surrogate). Modified THP nucleotides include, but are not limited to, what is referred to in the art as hexitol nucleic acid (HNA), anitol nucleic acid (ANA), manitol nucleic acid (MNA) (see, e.g., Leumann, Bioorg. Med. Chem., 2002, 10, 841-854), or fluoro HNA (F-HNA). In some embodiments of the invention, sugar surrogates comprise rings having more than 5 atoms and more than one heteroatom. For example nucleotides comprising morpholino sugar moieties and their use in oligomeric compounds has been reported (see for example: Braasch et al., Biochemistry, 2002, 41, 4503-4510; and U.S. Pat. Nos. 5,698,685; 5,166,315; 5,185,444; and 5,034,506). Morpholinos may be modified, for example by adding or altering various substituent groups from the above morpholino structure. Such sugar surrogates are referred to herein as “modified morpholinos.”

Combinations of modifications are also provided without limitation, such as 2′-F-5′-methyl substituted nucleosides (see PCT International Application WO 2008/101157 published on Aug. 21, 2008 for other disclosed 5′, 2′-bis substituted nucleosides) and replacement of the ribosyl ring oxygen atom with S and further substitution at the 2′-position (see published U.S. Patent Application US2005-0130923, published on Jun. 16, 2005) or alternatively 5′-substitution of a bicyclic nucleic acid (see PCT International Application WO 2007/134181, published on Nov. 22, 2007 wherein a 4′-CH2-0-2′ bicyclic nucleoside is further substituted at the 5′ position with a 5′-methyl or a 5′-vinyl group). The synthesis and preparation of carbocyclic bicyclic nucleosides along with their oligomerization and biochemical studies have also been described (see, e.g., Srivastava et al., J. Am. Chem. Soc. 2007, 129(26), 8362-8379).

In certain embodiments, a nucleic acid inhibitor comprises one or more modified cyclohexenyl nucleosides, which is a nucleoside having a six-membered cyclohexenyl in place of the pentofuranosyl residue in naturally occurring nucleosides. Modified cyclohexenyl nucleosides include, but are not limited to those described in the art (see for example commonly owned, published PCT Application WO 2010/036696, published on Apr. 10, 2010, Robeyns et al., J. Am. Chem. Soc., 2008, 130(6), 1979-1984; Horvath et al., Tetrahedron Letters, 2007, 48, 3621-3623; Nauwelaerts et al., J. Am. Chem. Soc., 2007, 129(30), 9340-9348; Gu et al., Nucleosides, Nucleotides & Nucleic Acids, 2005, 24(5-7), 993-998; Nauwelaerts et al., Nucleic Acids Research, 2005, 33(8), 2452-2463; Robeyns et al., Acta Crystallographica, Section F: Structural Biology and Crystallization Communications, 2005, F61(6), 585-586; Gu et al., Tetrahedron, 2004, 60(9), 2111-2123; Gu et al., Oligonucleotides, 2003, 13(6), 479-489; Wang et al., J. Org. Chem., 2003, 68, 4499-4505; Verbeure et al., Nucleic Acids Research, 2001, 29(24), 4941-4947; Wang et al., J. Org. Chem., 2001, 66, 8478-82; Wang et al., Nucleosides, Nucleotides & Nucleic Acids, 2001, 20(4-7), 785-788; Wang et al., J. Am. Chem., 2000, 122, 8595-8602; Published PCT application, WO 06/047842; and Published PCT Application WO 01/049687; the text of each is incorporated by reference herein, in their entirety).

A nucleic acid inhibitor of the invention can also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl anal other 8-substituted adenines and guanines, 5-halo, particularly 5-bromo, 5-trifluoromethyl and other 5-substituted uracils and cytosines, 7-methylguanine and 7-methyladenine, 8-azaguanine and 8-azaadenine, 7-deazaguanine and 7-daazaadenine and 3-deazaguanine and 3-deazaadenine. Further nucleobases include those disclosed in U.S. Pat. No. 3,687,808, those disclosed in Modified Nucleosides in Biochemistry, Biotechnology and Medicine, Herdewijn, P. ed. Wiley-VCH, 2008; those disclosed in The Concise Encyclopedia Of Polymer Science And Engineering, pages 858-859, Kroschwitz, J. L, ed. John Wiley & Sons, 1990, these disclosed by Englisch et al., (1991) Angewandte Chemie, International Edition, 30:613, and those disclosed by Sanghvi, Y S., Chapter 15, dsRNA Research and Applications, pages 289-302, Crooke, S. T. and Lebleu, B., Ed., CRC Press, 1993. Certain of these nucleobases are particularly useful for increasing the binding affinity of the oligomeric compounds featured in the invention. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., Eds., dsRNA Research and Applications, CRC Press, Boca Raton, 1993, pp. 276-278) and are exemplary base substitutions, even more particularly when combined with 2′-O-methoxyethyl sugar modifications.

Representative U.S. patents that teach the preparation of certain of the above noted modified nucleobases as well as other modified nucleobases include, but are not limited to, the above noted U.S. Pat. Nos. 3,687,808, 4,845,205; 5,130,30; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,681,941; 5,750,692; 6,015,886; 6,147,200; 6,166,197; 6,222,025; 6,235,887; 6,380,368; 6,528,640; 6,639,062; 6,617,438; 7,045,610; 7,427,672; and 7,495,088, the entire contents of each of which are hereby incorporated herein by reference.

A nucleic acid inhibitor of the invention can also be modified to include one or more locked nucleic acids (LNA). A locked nucleic acid is a nucleotide having a modified ribose moiety in which the ribose moiety comprises an extra bridge connecting the 2′ and 4′ carbons. This structure effectively “locks” the ribose in the 3′-endo structural conformation. The addition of locked nucleic acids to siRNAs has been shown to increase siRNA stability in serum, and to reduce off-target effects (Elmen, J. et al., (2005) Nucleic Acids Research 33(1):439-447; Mook, O R. et al., (2007) Mol Canc Ther 6(3):833-843; Grunweller, A. et al., (2003) Nucleic Acids Research 31(12):3185-3193).

A nucleic acid inhibitor of the invention can also be modified to include one or more bicyclic sugar moities. A “bicyclic sugar” is a furanosyl ring modified by a aring formed by the bridging of two carbons, whether adjacent or non-adjacent. A“bicyclic nucleoside” (“BNA”) is a nucleoside having a sugar moiety comprising a ring formed by bridging two carbon, whether adjacent or non-adjacent, of the sugar ring, thereby forming a bicyclic ring system. In certain embodiments, the bridge connects the 4′-carbon and the 2′-carbon of the sugar ring, optionally, via the 2′-acyclic oxygen atom. Thus, in some embodiments an agent of the invention may include one or more locked nucleic acids (LNA). A locked nucleic acid is a nucleotide having a modified ribose moiety in which the ribose moiety comprises an extra bridge connecting the 2′ and 4′ carbons. In other words, an LNA is a nucleotide comprising a bicyclic sugar moiety comprising a 4′-CH2-O-2′ bridge. This structure effectively “locks” the ribose in the 3′-endo structural conformation. The addition of locked nucleic acids to siRNAs has been shown to increase siRNA stability in serum, and to reduce off-target effects (Elmen, J. et al., (2005) Nucleic Acids Research 33(1):439-447; Mook, O R. et al., (2007) Mol Canc Ther 6(3):833-843; Grunweller, A. et al., (2003) Nucleic Acids Research 31(12):3185-3193). Examples of bicyclic nucleosides for use in the polynucleotides of the invention include without limitation nucleosides comprising a bridge between the 4′ and the 2′ ribosyl ring atoms. In certain embodiments, the antisense polynucleotide agents of the invention include one or more bicyclic nucleosides comprising a 4′ to 2′ bridge.

A locked nucleoside can be represented by the structure (omitting stereochemistry),

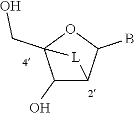

wherein B is a nucleobase or modified nucleobase and L is the linking group that joins the 2′-carbon to the 4′-carbon of the ribose ring. Examples of such 4′ to 2′ bridged bicyclic nucleosides, include but are not limited to 4′-(CH2)-O-2′ (LNA); 4′-(CH2)-S-2′; 4′-(CH2)2-O-2′ (ENA); 4′-CH(CH3)-O-2′ (also referred to as “constrained ethyl” or “cEt”) and 4′-CH(CH2OCH3)-O-2′ (and analogs thereof; see, e.g., U.S. Pat. No. 7,399,845); 4′-C(CH3)(CH3)-O-2′ (and analogs thereof; see e.g., U.S. Pat. No. 8,278,283); 4′-CH2-N(OCH3)-2′ (and analogs thereof; see e.g., U.S. Pat. No. 8,278,425); 4′-CH2-O—N(CH3)-2′ (see, e.g., U.S. Patent Publication No. 2004/0171570); 4′-CH2-N(R)—O-2′, wherein R is H, C1-C12 alkyl, or a nitrogen protecting group (see, e.g., U.S. Pat. No. 7,427,672); 4′-CH2-C(H)(CH3)-2′ (see, e.g., Chattopadhyaya et al., J. Org. Chem., 2009, 74, 118-134); and 4′-CH2-C(═CH2)-2′ (and analogs thereof; see, e.g., U.S. Pat. No. 8,278,426). The entire contents of each of the foregoing are hereby incorporated herein by reference.

Additional representative U.S. patents and US Patent Publications that teach the preparation of locked nucleic acid nucleotides include, but are not limited to, the following: U.S. Pat. Nos. 6,268,490; 6,525,191; 6,670,461; 6,770,748; 6,794,499; 6,998,484; 7,053,207; 7,034,133; 7,084,125; 7,399,845; 7,427,672; 7,569,686; 7,741,457; 8,022,193; 8,030,467; 8,278,425; 8,278,426; 8,278,283; US 2008/0039618; and US 2009/0012281, the entire contents of each of which are hereby incorporated herein by reference.

Any of the foregoing bicyclic nucleosides can be prepared having one or more stereochemical sugar configurations including for example α-L-ribofuranose and β-D-ribofuranose (see WO 99/14226).

A nucleic acid inhibitor of the invention can also be modified to include one or more constrained ethyl nucleotides. As used herein, a “constrained ethyl nucleotide” or “cEt” is a locked nucleic acid comprising a bicyclic sugar moiety comprising a 4′-CH(CH3)-0-2′ bridge (i.e., L in the preceding structure). In one embodiment, a constrained ethyl nucleotide is in the S conformation referred to herein as “S-cEt.”

A nucleic acid inhibitor of the invention may also include one or more “conformationally restricted nucleotides” (“CRN”). CRN are nucleotide analogs with a linker connecting the C2′ and C4′ carbons of ribose or the C3 and —C5′ carbons of ribose. CRN lock the ribose ring into a stable conformation and increase the hybridization affinity to mRNA. The linker is of sufficient length to place the oxygen in an optimal position for stability and affinity resulting in less ribose ring puckering.

Representative publications that teach the preparation of certain of the above noted CRN include, but are not limited to, US Patent Publication No. 2013/0190383; and PCT publication WO 2013/036868, the entire contents of each of which are hereby incorporated herein by reference.

In some embodiments, a nucleic acid inhibitor of the invention comprises one or more monomers that are UNA (unlocked nucleic acid) nucleotides. UNA is unlocked acyclic nucleic acid, wherein any of the bonds of the sugar has been removed, forming an unlocked “sugar” residue. In one example, UNA also encompasses monomer with bonds between C1′-C4′ have been removed (i.e. the covalent carbon-oxygen-carbon bond between the C1′ and C4′ carbons). In another example, the C2′-C3′ bond (i.e. the covalent carbon-carbon bond between the C2′ and C3′ carbons) of the sugar has been removed (see Nuc. Acids Symp. Series, 52, 133-134 (2008) and Fluiter et al., Mol. Biosyst., 2009, 10, 1039 hereby incorporated by reference).

Representative U.S. publications that teach the preparation of UNA include, but are not limited to, U.S. Pat. No. 8,314,227; and US Patent Publication Nos. 2013/0096289; 2013/0011922; and 2011/0313020, the entire contents of each of which are hereby incorporated herein by reference.

Potentially stabilizing modifications to the ends of nucleic acid inhibitors can include N-(acetylaminocaproyl)-4-hydroxyprolinol (Hyp-C6-NHAc), N-(caproyl-4-hydroxyprolinol (Hyp-C6), N-(acetyl-4-hydroxyprolinol (Hyp-NHAc), thymidine-2′-0-deoxythymidine (ether), N-(aminocaproyl)-4-hydroxyprolinol (Hyp-C6-amino), 2-docosanoyl-uridine-3″-phosphate, inverted base dT(idT) and others. Disclosure of this modification can be found in PCT Publication No. WO 2011/005861.

Other modifications of a nucleic acid inhibitor of the invention include a 5′ phosphate or 5′ phosphate mimic, e.g., a 5′-terminal phosphate or phosphate mimic on the antisense strand of an a nucleic acid inhibitor. Suitable phosphate mimics are disclosed in, for example US Patent Publication No. 2012/0157511, the entire contents of which are incorporated herein by reference.

Any of the nucleic acid inhibitors of the invention may be optionally conjugated with a ligand, such as a GalNAc derivative ligand, as described below.

As described in more detail below, a nucleic acid inhibitor that contains conjugations of one or more carbohydrate moieties to a nucleic acid inhibitor can optimize one or more properties of the inhibitor. In many cases, the carbohydrate moiety will be attached to a modified subunit of the nucleic acid inhibitor. For example, the ribose sugar of one or more ribonucleotide subunits of an inhibitor can be replaced with another moiety, e.g., a non-carbohydrate (such as, cyclic) carrier to which is attached a carbohydrate ligand. A ribonucleotide subunit in which the ribose sugar of the subunit has been so replaced is referred to herein as a ribose replacement modification subunit (RRMS). A cyclic carrier may be a carbocyclic ring system, i.e., all ring atoms are carbon atoms, or a heterocyclic ring system, i.e., one or more ring atoms may be a heteroatom, e.g., nitrogen, oxygen, sulfur. The cyclic carrier may be a monocyclic ring system, or may contain two or more rings, e.g. fused rings. The cyclic carrier may be a fully saturated ring system, or it may contain one or more double bonds.

The ligand may be attached to the nucleic acid inhibitor via a carrier. The carriers include (i) at least one “backbone attachment point,” such as, two “backbone attachment points” and (ii) at least one “tethering attachment point.” A “backbone attachment point” as used herein refers to a functional group, e.g. a hydroxyl group, or generally, a bond available for, and that is suitable for incorporation of the carrier into the backbone, e.g., the phosphate, or modified phosphate, e.g., sulfur containing, backbone, of a ribonucleic acid. A “tethering attachment point” (TAP) in some embodiments refers to a constituent ring atom of the cyclic carrier, e.g., a carbon atom or a heteroatom (distinct from an atom which provides a backbone attachment point), that connects a selected moiety. The moiety can be, e.g., a carbohydrate, e.g. monosaccharide, disaccharide, trisaccharide, tetrasaccharide, oligosaccharide and polysaccharide. Optionally, the selected moiety is connected by an intervening tether to the cyclic carrier. Thus, the cyclic carrier will often include a functional group, e.g., an amino group, or generally, provide a bond, that is suitable for incorporation or tethering of another chemical entity, e.g., a ligand to the constituent ring.

The nucleic acid inhibitors may be conjugated to a ligand via a carrier, wherein the carrier can be cyclic group or acyclic group; in some embodiments, the cyclic group is selected from pyrrolidinyl, pyrazolinyl, pyrazolidinyl, imidazolinyl, imidazolidinyl, piperidinyl, piperazinyl, [1,3]dioxolane, oxazolidinyl, isoxazolidinyl, morpholinyl, thiazolidinyl, isothiazolidinyl, quinoxalinyl, pyridazinonyl, tetrahydrofuryl and and decalin; in some embodiments, the acyclic group is selected from serinol backbone or diethanolamine backbone.

ii. Modified dsRNA Agents Comprising Motifs of the Invention

In certain aspects of the invention, the double stranded RNAi agents of the invention include agents with chemical modifications as disclosed, for example, in WO 2013/075035, filed on Nov. 16, 2012, the entire contents of which are incorporated herein by reference.

It is to be understood that, in embodiments in which a first dsRNA agent targeting LDHA and a second dsRNA agent targeting HAO1 are covalently attached (i.e., a dual targeting RNAi agent), the first agent may comprise any one or more of the motifs described below, the second agent may comprise any one or more of the motifs described below, or both the first agent and the second agent may independently comprise any one or more of the motifs described below.

Accordingly, the invention provides double stranded RNAi agents capable of inhibiting the expression of a target gene (i.e., an LDHA gene, an HAO1 gene, a PRODH2 gene, or both an LDHA gene and an HAO1 gene) in vivo. The RNAi agent comprises a sense strand and an antisense strand. Each strand of the RNAi agent may range from 12-30 nucleotides in length. For example, each strand may be between 14-30 nucleotides in length, 17-30 nucleotides in length, 25-30 nucleotides in length, 27-30 nucleotides in length, 17-23 nucleotides in length, 17-21 nucleotides in length, 17-19 nucleotides in length, 19-25 nucleotides in length, 19-23 nucleotides in length, 19-21 nucleotides in length, 21-25 nucleotides in length, or 21-23 nucleotides in length.

The sense strand and antisense strand typically form a duplex double stranded RNA (“dsRNA”), also referred to herein as an “RNAi agent.” The duplex region of an RNAi agent may be 12-30 nucleotide pairs in length. For example, the duplex region can be between 14-30 nucleotide pairs in length, 17-30 nucleotide pairs in length, 27-30 nucleotide pairs in length, 17-23 nucleotide pairs in length, 17-21 nucleotide pairs in length, 17-19 nucleotide pairs in length, 19-25 nucleotide pairs in length, 19-23 nucleotide pairs in length, 19-21 nucleotide pairs in length, 21-25 nucleotide pairs in length, or 21-23 nucleotide pairs in length. In another example, the duplex region is selected from 15, 16, 17, 18, 19, 20, 21, 22, 23, 24, 25, 26, and 27 nucleotides in length.

In one embodiment, the RNAi agent may contain one or more overhang regions and/or capping groups at the 3′-end, 5′-end, or both ends of one or both strands. The overhang can be 1-6 nucleotides in length, for instance 2-6 nucleotides in length, 1-5 nucleotides in length, 2-5 nucleotides in length, 1-4 nucleotides in length, 2-4 nucleotides in length, 1-3 nucleotides in length, 2-3 nucleotides in length, or 1-2 nucleotides in length. The overhangs can be the result of one strand being longer than the other, or the result of two strands of the same length being staggered. The overhang can form a mismatch with the target mRNA or it can be complementary to the gene sequences being targeted or can be another sequence. The first and second strands can also be joined, e.g., by additional bases to form a hairpin, or by other non-base linkers.

In one embodiment, the nucleotides in the overhang region of the RNAi agent can each independently be a modified or unmodified nucleotide including, but no limited to 2′-sugar modified, such as, 2-F, 2′-Omethyl, thymidine (T), 2′-O-methoxyethyl-5-methyluridine (Teo), 2′-O-methoxyethyladenosine (Aeo), 2′-O-methoxyethyl-5-methylcytidine (m5Ceo), and any combinations thereof. For example, TT can be an overhang sequence for either end on either strand. The overhang can form a mismatch with the target mRNA or it can be complementary to the gene sequences being targeted or can be another sequence.

The 5′- or 3′-overhangs at the sense strand, antisense strand or both strands of the RNAi agent may be phosphorylated. In some embodiments, the overhang region(s) contains two nucleotides having a phosphorothioate between the two nucleotides, where the two nucleotides can be the same or different. In one embodiment, the overhang is present at the 3′-end of the sense strand, antisense strand, or both strands. In one embodiment, this 3′-overhang is present in the antisense strand. In one embodiment, this 3′-overhang is present in the sense strand.

The RNAi agent may contain only a single overhang, which can strengthen the interference activity of the RNAi, without affecting its overall stability. For example, the single-stranded overhang may be located at the 3-terminal end of the sense strand or, alternatively, at the 3-terminal end of the antisense strand. The RNAi may also have a blunt end, located at the 5′-end of the antisense strand (or the 3′-end of the sense strand) or vice versa. Generally, the antisense strand of the RNAi has a nucleotide overhang at the 3′-end, and the 5′-end is blunt. While not wishing to be bound by theory, the asymmetric blunt end at the 5′-end of the antisense strand and 3′-end overhang of the antisense strand favor the guide strand loading into RISC process.

In one embodiment, the RNAi agent is a double blunt-ended of 19 nucleotides in length, wherein the sense strand contains at least one motif of three 2′-F modifications on three consecutive nucleotides at positions 7, 8, and 9 from the 5′end. The antisense strand contains at least one motif of three 2′-O-methyl modifications on three consecutive nucleotides at positions 11, 12, and 13 from the 5′end.

In another embodiment, the RNAi agent is a double blunt-ended of 20 nucleotides in length, wherein the sense strand contains at least one motif of three 2′-F modifications on three consecutive nucleotides at positions 8, 9, and 10 from the 5′end. The antisense strand contains at least one motif of three 2′-O-methyl modifications on three consecutive nucleotides at positions 11, 12, and 13 from the 5′end.

In yet another embodiment, the RNAi agent is a double blunt-ended of 21 nucleotides in length, wherein the sense strand contains at least one motif of three 2′-F modifications on three consecutive nucleotides at positions 9, 10, and 11 from the 5′end. The antisense strand contains at least one motif of three 2′-O-methyl modifications on three consecutive nucleotides at positions 11, 12, and 13 from the 5′end.

In one embodiment, the RNAi agent comprises a 21 nucleotide sense strand and a 23 nucleotide antisense strand, wherein the sense strand contains at least one motif of three 2′-F modifications on three consecutive nucleotides at positions 9, 10, and 11 from the 5′end; the antisense strand contains at least one motif of three 2′-O-methyl modifications on three consecutive nucleotides at positions 11, 12, and 13 from the 5′end, wherein one end of the RNAi agent is blunt, while the other end comprises a 2 nucleotide overhang. For example, the 2 nucleotide overhang is at the 3′-end of the antisense strand.

When the 2 nucleotide overhang is at the 3′-end of the antisense strand, there may be two phosphorothioate internucleotide linkages between the terminal three nucleotides, wherein two of the three nucleotides are the overhang nucleotides, and the third nucleotide is a paired nucleotide next to the overhang nucleotide. In one embodiment, the RNAi agent additionally has two phosphorothioate internucleotide linkages between the terminal three nucleotides at both the 5′-end of the sense strand and at the 5′-end of the antisense strand. In one embodiment, every nucleotide in the sense strand and the antisense strand of the RNAi agent, including the nucleotides that are part of the motifs are modified nucleotides. In one embodiment each residue is independently modified with a 2′-O-methyl or 2′-fluoro, e.g., in an alternating motif. Optionally, the RNAi agent further comprises a ligand (such as GalNAc₃).

In one embodiment, the RNAi agent comprises a sense and an antisense strand, wherein the sense strand is 25-30 nucleotide residues in length, wherein starting from the 5′ terminal nucleotide (position 1) positions 1 to 23 of the first strand comprise at least 8 ribonucleotides; the antisense strand is 36-66 nucleotide residues in length and, starting from the 3′ terminal nucleotide, comprises at least 8 ribonucleotides in the positions paired with positions 1-23 of sense strand to form a duplex; wherein at least the 3′ terminal nucleotide of antisense strand is unpaired with sense strand, and up to 6 consecutive 3′ terminal nucleotides are unpaired with sense strand, thereby forming a 3′ single stranded overhang of 1-6 nucleotides; wherein the 5′ terminus of antisense strand comprises from 10-30 consecutive nucleotides which are unpaired with sense strand, thereby forming a 10-30 nucleotide single stranded 5′ overhang; wherein at least the sense strand 5′ terminal and 3′ terminal nucleotides are base paired with nucleotides of antisense strand when sense and antisense strands are aligned for maximum complementarity, thereby forming a substantially duplexed region between sense and antisense strands; and antisense strand is sufficiently complementary to a target RNA along at least 19 ribonucleotides of antisense strand length to reduce target gene expression when the double stranded nucleic acid is introduced into a mammalian cell; and wherein the sense strand contains at least one motif of three 2′-F modifications on three consecutive nucleotides, where at least one of the motifs occurs at or near the cleavage site. The antisense strand contains at least one motif of three 2′-O-methyl modifications on three consecutive nucleotides at or near the cleavage site.

In one embodiment, the RNAi agent comprises sense and antisense strands, wherein the RNAi agent comprises a first strand having a length which is at least 25 and at most 29 nucleotides and a second strand having a length which is at most 30 nucleotides with at least one motif of three 2′-O-methyl modifications on three consecutive nucleotides at position 11, 12, and 13 from the 5′ end; wherein the 3′ end of the first strand and the 5′ end of the second strand form a blunt end and the second strand is 1-4 nucleotides longer at its 3′ end than the first strand, wherein the duplex region region which is at least 25 nucleotides in length, and the second strand is sufficiently complementary to a target mRNA along at least 19 nucleotide of the second strand length to reduce target gene expression when the RNAi agent is introduced into a mammalian cell, and wherein dicer cleavage of the RNAi agent results in an siRNA comprising the 3′ end of the second strand, thereby reducing expression of the target gene in the mammal. Optionally, the RNAi agent further comprises a ligand.

In one embodiment, the sense strand of the RNAi agent contains at least one motif of three identical modifications on three consecutive nucleotides, where one of the motifs occurs at the cleavage site in the sense strand.

In one embodiment, the antisense strand of the RNAi agent can also contain at least one motif of three identical modifications on three consecutive nucleotides, where one of the motifs occurs at or near the cleavage site in the antisense strand.

For an RNAi agent having a duplex region of 17-23 nucleotide in length, the cleavage site of the antisense strand is typically around the 10, 11 and 12 positions from the 5′-end. Thus the motifs of three identical modifications may occur at the 9, 10, and 11 positions; 10, 11, and 12 positions; 11, 12, and 13 positions; 12, 13, and 14 positions; or 13, 14, and 15 positions of the antisense strand, the count starting from the first nucleotide from the 5′-end of the antisense strand, or, the count starting from the first paired nucleotide within the duplex region from the 5′-end of the antisense strand. The cleavage site in the antisense strand may also change according to the length of the duplex region of the RNAi from the 5′-end.

The sense strand of the RNAi agent may contain at least one motif of three identical modifications on three consecutive nucleotides at the cleavage site of the strand; and the antisense strand may have at least one motif of three identical modifications on three consecutive nucleotides at or near the cleavage site of the strand. When the sense strand and the antisense strand form a dsRNA duplex, the sense strand and the antisense strand can be so aligned that one motif of the three nucleotides on the sense strand and one motif of the three nucleotides on the antisense strand have at least one nucleotide overlap, i.e., at least one of the three nucleotides of the motif in the sense strand forms a base pair with at least one of the three nucleotides of the motif in the antisense strand. Alternatively, at least two nucleotides may overlap, or all three nucleotides may overlap.

In one embodiment, the sense strand of the RNAi agent may contain more than one motif of three identical modifications on three consecutive nucleotides. The first motif may occur at or near the cleavage site of the strand and the other motifs may be a wing modification. The term “wing modification” herein refers to a motif occurring at another portion of the strand that is separated from the motif at or near the cleavage site of the same strand. The wing modification is either adjacent to the first motif or is separated by at least one or more nucleotides. When the motifs are immediately adjacent to each other then the chemistry of the motifs are distinct from each other and when the motifs are separated by one or more nucleotide than the chemistries can be the same or different. Two or more wing modifications may be present. For instance, when two wing modifications are present, each wing modification may occur at one end relative to the first motif which is at or near cleavage site or on either side of the lead motif.

Like the sense strand, the antisense strand of the RNAi agent may contain more than one motifs of three identical modifications on three consecutive nucleotides, with at least one of the motifs occurring at or near the cleavage site of the strand. This antisense strand may also contain one or more wing modifications in an alignment similar to the wing modifications that may be present on the sense strand.

In one embodiment, the wing modification on the sense strand or antisense strand of the RNAi agent typically does not include the first one or two terminal nucleotides at the 3′-end, 5′-end or both ends of the strand.

In another embodiment, the wing modification on the sense strand or antisense strand of the RNAi agent typically does not include the first one or two paired nucleotides within the duplex region at the 3′-end, 5′-end or both ends of the strand.

When the sense strand and the antisense strand of the RNAi agent each contain at least one wing modification, the wing modifications may fall on the same end of the duplex region, and have an overlap of one, two or three nucleotides.

When the sense strand and the antisense strand of the RNAi agent each contain at least two wing modifications, the sense strand and the antisense strand can be so aligned that two modifications each from one strand fall on one end of the duplex region, having an overlap of one, two or three nucleotides; two modifications each from one strand fall on the other end of the duplex region, having an overlap of one, two or three nucleotides; two modifications one strand fall on each side of the lead motif, having an overlap of one, two or three nucleotides in the duplex region.

In one embodiment, every nucleotide in the sense strand and antisense strand of the RNAi agent, including the nucleotides that are part of the motifs, may be modified. Each nucleotide may be modified with the same or different modification which can include one or more alteration of one or both of the non-linking phosphate oxygens and/or of one or more of the linking phosphate oxygens; alteration of a constituent of the ribose sugar, e.g., of the 2′ hydroxyl on the ribose sugar; wholesale replacement of the phosphate moiety with “dephospho” linkers; modification or replacement of a naturally occurring base; and replacement or modification of the ribose-phosphate backbone.

As nucleic acids are polymers of subunits, many of the modifications occur at a position which is repeated within a nucleic acid, e.g., a modification of a base, or a phosphate moiety, or a non-linking O of a phosphate moiety. In some cases the modification will occur at all of the subject positions in the nucleic acid but in many cases it will not. By way of example, a modification may only occur at a 3′ or 5′ terminal position, may only occur in a terminal region, e.g., at a position on a terminal nucleotide or in the last 2, 3, 4, 5, or 10 nucleotides of a strand. A modification may occur in a double strand region, a single strand region, or in both. A modification may occur only in the double strand region of a RNA or may only occur in a single strand region of a RNA. For example, a phosphorothioate modification at a non-linking O position may only occur at one or both termini, may only occur in a terminal region, e.g., at a position on a terminal nucleotide or in the last 2, 3, 4, 5, or 10 nucleotides of a strand, or may occur in double strand and single strand regions, particularly at termini. The 5′ end or ends can be phosphorylated.

It may be possible, e.g., to enhance stability, to include particular bases in overhangs, or to include modified nucleotides or nucleotide surrogates, in single strand overhangs, e.g., in a 5′ or 3′ overhang, or in both. For example, it can be desirable to include purine nucleotides in overhangs. In some embodiments all or some of the bases in a 3′ or 5′ overhang may be modified, e.g., with a modification described herein. Modifications can include, e.g., the use of modifications at the 2′ position of the ribose sugar with modifications that are known in the art, e.g., the use of deoxyribonucleotides, 2′-deoxy-2′-fluoro (2′-F) or 2′-O-methyl modified instead of the ribosugar of the nucleobase, and modifications in the phosphate group, e.g., phosphorothioate modifications. Overhangs need not be homologous with the target sequence.

In one embodiment, each residue of the sense strand and antisense strand is independently modified with LNA, CRN, cET, UNA, HNA, CeNA, 2′-methoxyethyl, 2′-O-methyl, 2′-O-allyl, 2′-C-allyl, 2′-deoxy, 2′-hydroxyl, or 2′-fluoro. The strands can contain more than one modification. In one embodiment, each residue of the sense strand and antisense strand is independently modified with 2′-O-methyl or 2′-fluoro.

At least two different modifications are typically present on the sense strand and antisense strand. Those two modifications may be the 2′-O-methyl or 2′-fluoro modifications, or others. In one embodiment, the Nₐ and/or N_{b} comprise modifications of an alternating pattern. The term “alternating motif” as used herein refers to a motif having one or more modifications, each modification occurring on alternating nucleotides of one strand. The alternating nucleotide may refer to one per every other nucleotide or one per every three nucleotides, or a similar pattern. For example, if A, B and C each represent one type of modification to the nucleotide, the alternating motif can be “ABABABABABAB . . . ,” “AABBAABBAABB . . . ,” “AABAABAABAAB . . . ,” “AAABAAABAAAB . . . ,” “AAABBBAAABBB . . . ,” or “ABCABCABCABC . . . ,” etc. The type of modifications contained in the alternating motif may be the same or different. For example, if A, B, C, D each represent one type of modification on the nucleotide, the alternating pattern, i.e., modifications on every other nucleotide, may be the same, but each of the sense strand or antisense strand can be selected from several possibilities of modifications within the alternating motif such as “ABABAB . . . ”, “ACACAC . . . ” “BDBDBD . . . ” or “CDCDCD . . . ,” etc.

In one embodiment, the RNAi agent of the invention comprises the modification pattern for the alternating motif on the sense strand relative to the modification pattern for the alternating motif on the antisense strand is shifted. The shift may be such that the modified group of nucleotides of the sense strand corresponds to a differently modified group of nucleotides of the antisense strand and vice versa. For example, the sense strand when paired with the antisense strand in the dsRNA duplex, the alternating motif in the sense strand may start with “ABABAB” from 5′-3′ of the strand and the alternating motif in the antisense strand may start with “BABABA” from 5′-3′ of the strand within the duplex region. As another example, the alternating motif in the sense strand may start with “AABBAABB” from 5′-3′ of the strand and the alternating motif in the antisense strand may start with “BBAABBAA” from 5′-3′ of the strand within the duplex region, so that there is a complete or partial shift of the modification patterns between the sense strand and the antisense strand.

In one embodiment, the RNAi agent comprises the pattern of the alternating motif of 2′-O-methyl modification and 2′-F modification on the sense strand initially has a shift relative to the pattern of the alternating motif of 2′-O-methyl modification and 2′-F modification on the antisense strand initially, i.e., the 2′-O-methyl modified nucleotide on the sense strand base pairs with a 2′-F modified nucleotide on the antisense strand and vice versa. The 1 position of the sense strand may start with the 2′-F modification, and the 1 position of the antisense strand may start with the 2′-O-methyl modification.

The introduction of one or more motifs of three identical modifications on three consecutive nucleotides to the sense strand and/or antisense strand interrupts the initial modification pattern present in the sense strand and/or antisense strand. This interruption of the modification pattern of the sense and/or antisense strand by introducing one or more motifs of three identical modifications on three consecutive nucleotides to the sense and/or antisense strand surprisingly enhances the gene silencing activity to the target gene.

In one embodiment, when the motif of three identical modifications on three consecutive nucleotides is introduced to any of the strands, the modification of the nucleotide next to the motif is a different modification than the modification of the motif. For example, the portion of the sequence containing the motif is “ . . . NₐYYYN_{b} . . . ,” where “Y” represents the modification of the motif of three identical modifications on three consecutive nucleotide, and “Nₐ” and “N_{b}” represent a modification to the nucleotide next to the motif “YYY” that is different than the modification of Y, and where Nₐ and N_{b} can be the same or different modifications. Alternatively, Nₐ and/or N_{b} may be present or absent when there is a wing modification present.

The RNAi agent may further comprise at least one phosphorothioate or methylphosphonate internucleotide linkage. The phosphorothioate or methylphosphonate internucleotide linkage modification may occur on any nucleotide of the sense strand or antisense strand or both strands in any position of the strand. For instance, the internucleotide linkage modification may occur on every nucleotide on the sense strand and/or antisense strand; each internucleotide linkage modification may occur in an alternating pattern on the sense strand and/or antisense strand; or the sense strand or antisense strand may contain both internucleotide linkage modifications in an alternating pattern. The alternating pattern of the internucleotide linkage modification on the sense strand may be the same or different from the antisense strand, and the alternating pattern of the internucleotide linkage modification on the sense strand may have a shift relative to the alternating pattern of the internucleotide linkage modification on the antisense strand. In one embodiment, a double-stranded RNAi agent comprises 6-8 phosphorothioate internucleotide linkages. In one embodiment, the antisense strand comprises two phosphorothioate internucleotide linkages at the 5′-terminus and two phosphorothioate internucleotide linkages at the 3′-terminus, and the sense strand comprises at least two phosphorothioate internucleotide linkages at either the 5′-terminus or the 3′-terminus.

In one embodiment, the RNAi comprises a phosphorothioate or methylphosphonate internucleotide linkage modification in the overhang region. For example, the overhang region may contain two nucleotides having a phosphorothioate or methylphosphonate internucleotide linkage between the two nucleotides. Internucleotide linkage modifications also may be made to link the overhang nucleotides with the terminal paired nucleotides within the duplex region. For example, at least 2, 3, 4, or all the overhang nucleotides may be linked through phosphorothioate or methylphosphonate internucleotide linkage, and optionally, there may be additional phosphorothioate or methylphosphonate internucleotide linkages linking the overhang nucleotide with a paired nucleotide that is next to the overhang nucleotide. For instance, there may be at least two phosphorothioate internucleotide linkages between the terminal three nucleotides, in which two of the three nucleotides are overhang nucleotides, and the third is a paired nucleotide next to the overhang nucleotide. These terminal three nucleotides may be at the 3′-end of the antisense strand, the 3′-end of the sense strand, the 5′-end of the antisense strand, and/or the 5′end of the antisense strand.

In one embodiment, the 2 nucleotide overhang is at the 3′-end of the antisense strand, and there are two phosphorothioate internucleotide linkages between the terminal three nucleotides, wherein two of the three nucleotides are the overhang nucleotides, and the third nucleotide is a paired nucleotide next to the overhang nucleotide. Optionally, the RNAi agent may additionally have two phosphorothioate internucleotide linkages between the terminal three nucleotides at both the 5′-end of the sense strand and at the 5′-end of the antisense strand.

In one embodiment, the RNAi agent comprises mismatch(es) with the target, within the duplex, or combinations thereof. The mismatch may occur in the overhang region or the duplex region. The base pair may be ranked on the basis of their propensity to promote dissociation or melting (e.g., on the free energy of association or dissociation of a particular pairing, the simplest approach is to examine the pairs on an individual pair basis, though next neighbor or similar analysis can also be used). In terms of promoting dissociation: A:U is preferred over G:C; G:U is preferred over G:C; and I:C is preferred over G:C (I=inosine). Mismatches, e.g., non-canonical or other than canonical pairings (as described elsewhere herein) are preferred over canonical (A:T, A:U, G:C) pairings; and pairings which include a universal base are preferred over canonical pairings.

In one embodiment, the RNAi agent comprises at least one of the first 1, 2, 3, 4, or 5 base pairs within the duplex regions from the 5′-end of the antisense strand independently selected from the group of: A:U, G:U, I:C, and mismatched pairs, e.g., non-canonical or other than canonical pairings or pairings which include a universal base, to promote the dissociation of the antisense strand at the 5′-end of the duplex.

In one embodiment, the nucleotide at the 1 position within the duplex region from the 5′-end in the antisense strand is selected from the group consisting of A, dA, dU, U, and dT. Alternatively, at least one of the first 1, 2 or 3 base pair within the duplex region from the 5′-end of the antisense strand is an AU base pair. For example, the first base pair within the duplex region from the 5′-end of the antisense strand is an AU base pair.

In another embodiment, the nucleotide at the 3′-end of the sense strand is deoxythimidine (dT). In another embodiment, the nucleotide at the 3′-end of the antisense strand is deoxythimidine (dT). In one embodiment, there is a short sequence of deoxy-thymine nucleotides, for example, two dT nucleotides on the 3′-end of the sense and/or antisense strand.

In one embodiment, the sense strand sequence may be represented by formula (I):

(I)
5′ nₚ-Nₐ-(X X X)ᵢ-N_{b}-Y Y Y-N_{b}-(Z Z Z)ⱼ-Nₐ-n_{q} 3′

wherein:
i and j are each independently 0 or 1;
p and q are each independently 0-6;
each Nₐ independently represents an oligonucleotide sequence comprising 0-25 modified nucleotides, each sequence comprising at least two differently modified nucleotides;
each N_{b} independently represents an oligonucleotide sequence comprising 0-10 modified nucleotides;
each nₚ and n_{q} independently represent an overhang nucleotide;
wherein Nb and Y do not have the same modification; and
XXX, YYY and ZZZ each independently represent one motif of three identical modifications on three consecutive nucleotides. In some embodiments, YYY is all 2′-F modified nucleotides.
   In one embodiment, the Nₐ and/or N_{b} comprise modifications of alternating pattern.

In one embodiment, the YYY motif occurs at or near the cleavage site of the sense strand. For example, when the RNAi agent has a duplex region of 17-23 nucleotides in length, the YYY motif can occur at or the vicinity of the cleavage site (e.g.: can occur at positions 6, 7, 8, 7, 8, 9, 8, 9, 10, 9, 10, 11, 10, 11, 12 or 11, 12, 13) of—the sense strand, the count starting from the 1^{st} nucleotide, from the 5′-end; or optionally, the count starting at the 1^{st} paired nucleotide within the duplex region, from the 5′-end.

In one embodiment, i is 1 and j is 0, or i is 0 and j is 1, or both i and j are 1. The sense strand can therefore be represented by the following formulas:

(Ib)
5′ nₚ-Nₐ-YYY-N_{b}-ZZZ-Nₐ-n_{q} 3′;
(Ic)
5′ nₚ-Nₐ-XXX-N_{b}-YYY-Nₐ-n_{q} 3′;
or
(Id)
5′ nₚ-Nₐ-XXX-N_{b}-YYY-N_{b}-ZZZ-Nₐ-n_{q} 3′.

When the sense strand is represented by formula (Ib), N_{b} represents an oligonucleotide sequence comprising 0-10, 0-7, 0-5, 0-4, 0-2 or 0 modified nucleotides. Each Nₐ independently can represent an oligonucleotide sequence comprising 2-20, 2-15, or 2-10 modified nucleotides.

When the sense strand is represented as formula (Ic), N_{b} represents an oligonucleotide sequence comprising 0-10, 0-7, 0-10, 0-7, 0-5, 0-4, 0-2 or 0 modified nucleotides. Each Nₐ can independently represent an oligonucleotide sequence comprising 2-20, 2-15, or 2-10 modified nucleotides.

When the sense strand is represented as formula (Id), each N_{b} independently represents an oligonucleotide sequence comprising 0-10, 0-7, 0-5, 0-4, 0-2 or 0 modified nucleotides. In some embodiment, N_{b} is 0, 1, 2, 3, 4, 5 or 6. Each Nₐ can independently represent an oligonucleotide sequence comprising 2-20, 2-15, or 2-10 modified nucleotides.

Each of X, Y and Z may be the same or different from each other.

In other embodiments, i is 0 and j is 0, and the sense strand may be represented by the formula:

(Ia)
5′ nₚ-Nₐ-YYY-Nₐ-n_{q} 3′.

When the sense strand is represented by formula (Ia), each Nₐ independently can represent an oligonucleotide sequence comprising 2-20, 2-15, or 2-10 modified nucleotides.

In one embodiment, the antisense strand sequence of the RNAi may be represented by formula (II):

(II)
5′ n_{q′}-Nₐ′-(Z′Z′Z′)ₖ-N_{b}′-Y′Y′Y′-N_{b}′-(X′X′X′)ₗ-N′ₐ-
nₚ′ 3′

wherein:
k and l are each independently 0 or 1;
p′ and q′ are each independently 0-6;

each Nₐ′ independently represents an oligonucleotide sequence comprising 0-25 modified nucleotides, each sequence comprising at least two differently modified nucleotides;

each N_{b}′ independently represents an oligonucleotide sequence comprising 0-10 modified nucleotides;

each nₚ′ and n_{q}′ independently represent an overhang nucleotide;

wherein N_{b}′ and Y′ do not have the same modification; and

X′X′X′, Y′Y′Y′ and Z′Z′Z′ each independently represent one motif of three identical modifications on three consecutive nucleotides.

In one embodiment, the Nₐ′ and/or N_{b}′ comprise modifications of alternating pattern.

The Y′Y′Y′ motif occurs at or near the cleavage site of the antisense strand. For example, when the RNAi agent has a duplex region of 17-23 nucleotide in length, the Y′Y′Y′ motif can occur at positions 9, 10, 11; 10, 11, 12; 11, 12, 13; 12, 13, 14; or 13, 14, 15 of the antisense strand, with the count starting from the 1^{st} nucleotide, from the 5′-end; or optionally, the count starting at the 1^{st} paired nucleotide within the duplex region, from the 5′-end. In some embodiments, the Y′Y′Y′ motif occurs at positions 11, 12, 13.

In one embodiment, Y′Y′Y′ motif is all 2′-OMe modified nucleotides.

In one embodiment, k is 1 and l is 0, or k is 0 and l is 1, or both k and l are 1.

The antisense strand can therefore be represented by the following formulas:

(IIb)
5′ n_{q′}-Nₐ′-Z′Z′Z′-N_{b}′-Y′Y′Y′-Nₐ′-n_{p′} 3′;
(IIc)
5′ n_{q′}-Nₐ′-Y′Y′Y′-N_{b}′-X′X′X′-n_{p′} 3′;
or
(IId)
5′ n_{q′}-Nₐ′-Z′Z′Z′-N_{b}′-Y′Y′Y′-N_{b}′-X′X′X′-Nₐ′-n_{p′} 3′.

When the antisense strand is represented by formula (IIb), N_{b}′ represents an oligonucleotide sequence comprising 0-10, 0-7, 0-10, 0-7, 0-5, 0-4, 0-2 or 0 modified nucleotides. Each Nₐ′ independently represents an oligonucleotide sequence comprising 2-20, 2-15, or 2-10 modified nucleotides.

When the antisense strand is represented as formula (IIc), N_{b}′ represents an oligonucleotide sequence comprising 0-10, 0-7, 0-10, 0-7, 0-5, 0-4, 0-2 or 0 modified nucleotides. Each Nₐ′ independently represents an oligonucleotide sequence comprising 2-20, 2-15, or 2-10 modified nucleotides.

When the antisense strand is represented as formula (IId), each N_{b}′ independently represents an oligonucleotide sequence comprising 0-10, 0-7, 0-10, 0-7, 0-5, 0-4, 0-2 or 0 modified nucleotides. Each Nₐ′ independently represents an oligonucleotide sequence comprising 2-20, 2-15, or 2-10 modified nucleotides. In some embodiments, N_{b} is 0, 1, 2, 3, 4, 5 or 6.

In other embodiments, k is 0 and l is 0 and the antisense strand may be represented by the formula:

(Ia)
5′ n_{p′}-N_{a′}-Y′Y′Y′-N_{a′}-n_{q′} 3′.

When the antisense strand is represented as formula (IIa), each Nₐ′ independently represents an oligonucleotide sequence comprising 2-20, 2-15, or 2-10 modified nucleotides.

Each of X′, Y′ and Z′ may be the same or different from each other.
Each nucleotide of the sense strand and antisense strand may be independently modified with LNA, CRN, UNA, cEt, HNA, CeNA, 2′-methoxyethyl, 2′-O-methyl, 2′-O-allyl, 2′-C-allyl, 2′-hydroxyl, or 2′-fluoro. For example, each nucleotide of the sense strand and antisense strand is independently modified with 2′-O-methyl or 2′-fluoro. Each X, Y, Z, X′, Y′ and Z′, in particular, may represent a 2′-O-methyl modification or a 2′-fluoro modification.

In one embodiment, the sense strand of the RNAi agent may contain YYY motif occurring at 9, 10 and 11 positions of the strand when the duplex region is 21 nt, the count starting from the 1^{st} nucleotide from the 5′-end, or optionally, the count starting at the 1^{st} paired nucleotide within the duplex region, from the 5′-end; and Y represents 2′-F modification. The sense strand may additionally contain XXX motif or ZZZ motifs as wing modifications at the opposite end of the duplex region; and XXX and ZZZ each independently represents a 2′-OMe modification or 2′-F modification.

In one embodiment the antisense strand may contain Y′Y′Y′ motif occurring at positions 11, 12, 13 of the strand, the count starting from the 1st nucleotide from the 5′ end, or optionally, the count starting at the 1st paired nucleotide within the duplex region, from the 5′-end; and Y′ represents 2′-O-methyl modification. The antisense strand may additionally contain X′X′X′ motif or Z′Z′Z′ motifs as wing modifications at the opposite end of the duplex region; and X′X′X′ and Z′Z′Z′ each independently represents a 2′-OMe modification or 2′-F modification.

The sense strand represented by any one of the above formulas (Ia), (Ib), (Ic), and (Id) forms a duplex with a antisense strand being represented by any one of formulas (IIa), (IIb), (IIc), and (IId), respectively.

Accordingly, the RNAi agents for use in the methods of the invention may comprise a sense strand and an antisense strand, each strand having 14 to 30 nucleotides, the RNAi duplex represented by formula (III):

(III)
sense:
5′ np-Na-(X X X)i-Nb-Y Y Y-Nb-(Z Z Z)j-Na-nq 3′
antisense:
3′ np′-Na′-(X′X′X′)k-Nb′-Y′Y′Y′-Nb′-(Z′Z′Z′)l-Na′-
nq′ 5′

wherein:
i, j, k, and l are each independently 0 or 1;
p, p′, q, and q′ are each independently 0-6;
   each Na and Na′ independently represents an oligonucleotide sequence comprising 0-25 modified nucleotides, each sequence comprising at least two differently modified nucleotides;
   each Nb and Nb′ independently represents an oligonucleotide sequence comprising 0-10 modified nucleotides;
   wherein each np′, np, nq′, and nq, each of which may or may not be present, independently represents an overhang nucleotide; and
   XXX, YYY, ZZZ, X′X′X′, Y′Y′Y′, and Z′Z′Z′ each independently represent one motif of three identical modifications on three consecutive nucleotides.

In one embodiment, i is 0 and j is 0; or i is 1 and j is 0; or i is 0 and j is 1; or both i and j are 0; or both i and j are 1. In another embodiment, k is 0 and l is 0; or k is 1 and l is 0; k is 0 and l is 1; or both k and l are 0; or both k and l are 1.

Exemplary combinations of the sense strand and antisense strand forming a RNAi duplex include the formulas below:

(IIIa)
5′ np-Na-Y Y Y-Na-nq 3′
3′ np′-Na′-Y′Y′Y′-Na′nq′ 5′
(IIIb)
5′ np-Na-Y Y Y-Nb-Z Z Z-Na-nq 3′
3′ np′-Na′-Y′Y′Y′-Nb′-Z′Z′Z′-Na′nq′ 5′
(IIIc)
5′ np-Na-X X X-Nb-Y Y Y-Na-nq 3′
3′ np′-Na′-X′X′X′-Nb′-Y′Y′Y′-Na′-nq′ 5′
(IIId)
5′ np-Na-X X X-Nb-Y Y Y-Nb-Z Z Z-Na-nq 3′
3′ np′-Na′-X′X′X′-Nb′-Y′Y′Y′-Nb′-Z′Z′Z′-Na-nq′ 5′

When the RNAi agent is represented by formula (IIIa), each Na independently represents an oligonucleotide sequence comprising 2-20, 2-15, or 2-10 modified nucleotides.

When the RNAi agent is represented by formula (IIIb), each Nb independently represents an oligonucleotide sequence comprising 1-10, 1-7, 1-5 or 1-4 modified nucleotides. Each Na independently represents an oligonucleotide sequence comprising 2-20, 2-15, or 2-10 modified nucleotides.

When the RNAi agent is represented as formula (IIIc), each Nb, Nb′ independently represents an oligonucleotide sequence comprising 0-10, 0-7, 0-10, 0-7, 0-5, 0-4, 0-2 or 0 modified nucleotides. Each Na independently represents an oligonucleotide sequence comprising 2-20, 2-15, or 2-10 modified nucleotides.

When the RNAi agent is represented as formula (IIId), each Nb, Nb′ independently represents an oligonucleotide sequence comprising 0-10, 0-7, 0-10, 0-7, 0-5, 0-4, 0-2 or 0 modified nucleotides. Each Na, Na′ independently represents an oligonucleotide sequence comprising 2-20, 2-15, or 2-10 modified nucleotides. Each of Na, Na′, Nb and Nb′ independently comprises modifications of alternating pattern.

Each of X, Y and Z in formulas (III), (IIIa), (IIIb), (IIIc), and (IIId) may be the same or different from each other.

When the RNAi agent is represented by formula (III), (IIIa), (IIIb), (IIIc), and (IIId), at least one of the Y nucleotides may form a base pair with one of the Y′ nucleotides. Alternatively, at least two of the Y nucleotides form base pairs with the corresponding Y′ nucleotides; or all three of the Y nucleotides all form base pairs with the corresponding Y′ nucleotides.

When the RNAi agent is represented by formula (IIIb) or (IIId), at least one of the Z nucleotides may form a base pair with one of the Z′ nucleotides. Alternatively, at least two of the Z nucleotides form base pairs with the corresponding Z′ nucleotides; or all three of the Z nucleotides all form base pairs with the corresponding Z′ nucleotides.

When the RNAi agent is represented as formula (IIIc) or (IIId), at least one of the X nucleotides may form a base pair with one of the X′ nucleotides. Alternatively, at least two of the X nucleotides form base pairs with the corresponding X′ nucleotides; or all three of the X nucleotides all form base pairs with the corresponding X′ nucleotides.

In one embodiment, the modification on the Y nucleotide is different than the modification on the Y′ nucleotide, the modification on the Z nucleotide is different than the modification on the Z′ nucleotide, and/or the modification on the X nucleotide is different than the modification on the X′ nucleotide.

In one embodiment, when the RNAi agent is represented by formula (IIId), the Na modifications are 2′-O-methyl or 2′-fluoro modifications. In another embodiment, when the RNAi agent is represented by formula (IIId), the Na modifications are 2′-O-methyl or 2′-fluoro modifications and np′>0 and at least one np′ is linked to a neighboring nucleotide a via phosphorothioate linkage. In yet another embodiment, when the RNAi agent is represented by formula (IIId), the Na modifications are 2′-O-methyl or 2′-fluoro modifications, np′>0 and at least one np′ is linked to a neighboring nucleotide via phosphorothioate linkage, and the sense strand is conjugated to one or more GalNAc derivatives attached through a bivalent or trivalent branched linker (described below). In another embodiment, when the RNAi agent is represented by formula (IIId), the Na modifications are 2′-O-methyl or 2′-fluoro modifications, np′>0 and at least one np′ is linked to a neighboring nucleotide via phosphorothioate linkage, the sense strand comprises at least one phosphorothioate linkage, and the sense strand is conjugated to one or more GalNAc derivatives attached through a bivalent or trivalent branched linker.

In one embodiment, when the RNAi agent is represented by formula (IIIa), the Na modifications are 2′-O-methyl or 2′-fluoro modifications, np′>0 and at least one np′ is linked to a neighboring nucleotide via phosphorothioate linkage, the sense strand comprises at least one phosphorothioate linkage, and the sense strand is conjugated to one or more GalNAc derivatives attached through a bivalent or trivalent branched linker.

In one embodiment, the RNAi agent is a multimer containing at least two duplexes represented by formula (III), (IIIa), (IIIb), (IIIc), and (IIId), wherein the duplexes are connected by a linker. The linker can be cleavable or non-cleavable. Optionally, the multimer further comprises a ligand. Each of the duplexes can target the same gene or two different genes; or each of the duplexes can target same gene at two different target sites.

In one embodiment, the RNAi agent is a multimer containing three, four, five, six or more duplexes represented by formula (III), (IIIa), (IIIb), (IIIc), and (IIId), wherein the duplexes are connected by a linker. The linker can be cleavable or non-cleavable. Optionally, the multimer further comprises a ligand. Each of the duplexes can target the same gene or two different genes; or each of the duplexes can target same gene at two different target sites.

In one embodiment, two RNAi agents represented by formula (III), (IIIa), (IIIb), (IIIc), and (IIId) are linked to each other at the 5′ end, and one or both of the 3′ ends and are optionally conjugated to to a ligand. Each of the agents can target the same gene or two different genes; or each of the agents can target same gene at two different target sites.

In certain embodiments, an RNAi agent of the invention may contain a low number of nucleotides containing a 2′-fluoro modification, e.g., 10 or fewer nucleotides with 2′-fluoro modification. For example, the RNAi agent may contain 10, 9, 8, 7, 6, 5, 4, 3, 2, 1 or 0 nucleotides with a 2′-fluoro modification. In a specific embodiment, the RNAi agent of the invention contains 10 nucleotides with a 2′-fluoro modification, e.g., 4 nucleotides with a 2′-fluoro modification in the sense strand and 6 nucleotides with a 2′-fluoro modification in the antisense strand. In another specific embodiment, the RNAi agent of the invention contains 6 nucleotides with a 2′-fluoro modification, e.g., 4 nucleotides with a 2′-fluoro modification in the sense strand and 2 nucleotides with a 2′-fluoro modification in the antisense strand.

In other embodiments, an RNAi agent of the invention may contain an ultra low number of nucleotides containing a 2′-fluoro modification, e.g., 2 or fewer nucleotides containing a 2′-fluoro modification. For example, the RNAi agent may contain 2, 1 of 0 nucleotides with a 2′-fluoro modification. In a specific embodiment, the RNAi agent may contain 2 nucleotides with a 2′-fluoro modification, e.g., 0 nucleotides with a 2-fluoro modification in the sense strand and 2 nucleotides with a 2′-fluoro modification in the antisense strand.

Various publications describe multimeric RNAi agents that can be used in the methods of the invention. Such publications include WO2007/091269, U.S. Pat. No. 7,858,769, WO2010/141511, WO2007/117686, WO2009/014887 and WO2011/031520 the entire contents of each of which are hereby incorporated herein by reference.

In certain embodiments, the compositions and methods of the disclosure include a vinyl phosphonate (VP) modification of an RNAi agent as described herein. In exemplary embodiments, a 5′-vinyl phosphonate modified nucleotide of the disclosure has the structure:

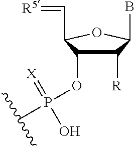

wherein X is O or S;
R is hydrogen, hydroxy, fluoro, or C₁₋₂₀alkoxy (e.g., methoxy or n-hexadecyloxy);
R^{5′} is ═C(H)—P(O)(OH)₂ and the double bond between the C5′ carbon and R^{5′} is in the E or Z orientation (e.g., E orientation); and
B is a nucleobase or a modified nucleobase, optionally where B is adenine, guanine, cytosine, thymine, or uracil.

A vinyl phosphonate of the instant disclosure may be attached to either the antisense or the sense strand of a dsRNA of the disclosure. In certain embodiments, a vinyl phosphonate of the instant disclosure is attached to the antisense strand of a dsRNA, optionally at the 5′ end of the antisense strand of the dsRNA.

Vinyl phosphonate modifications are also contemplated for the compositions and methods of the instant disclosure. An exemplary vinyl phosphonate structure includes the preceding structure, where R^{5′} is ═C(H)—OP(O)(OH)₂ and the double bond between the C5′ carbon and R^{5′} is in the E or Z orientation (e.g., E orientation).

As described in more detail below, the RNAi agent that contains conjugations of one or more carbohydrate moieties to a RNAi agent can optimize one or more properties of the RNAi agent. In many cases, the carbohydrate moiety will be attached to a modified subunit of the RNAi agent. For example, the ribose sugar of one or more ribonucleotide subunits of a dsRNA agent can be replaced with another moiety, e.g., a non-carbohydrate (such as, cyclic) carrier to which is attached a carbohydrate ligand. A ribonucleotide subunit in which the ribose sugar of the subunit has been so replaced is referred to herein as a ribose replacement modification subunit (RRMS). A cyclic carrier may be a carbocyclic ring system, i.e., all ring atoms are carbon atoms, or a heterocyclic ring system, i.e., one or more ring atoms may be a heteroatom, e.g., nitrogen, oxygen, sulfur. The cyclic carrier may be a monocyclic ring system, or may contain two or more rings, e.g. fused rings. The cyclic carrier may be a fully saturated ring system, or it may contain one or more double bonds.

The ligand may be attached to the polynucleotide via a carrier. The carriers include (i) at least one “backbone attachment point,” such as, two “backbone attachment points” and (ii) at least one “tethering attachment point.” A “backbone attachment point” as used herein refers to a functional group, e.g. a hydroxyl group, or generally, a bond available for, and that is suitable for incorporation of the carrier into the backbone, e.g., the phosphate, or modified phosphate, e.g., sulfur containing, backbone, of a ribonucleic acid. A “tethering attachment point” (TAP) in some embodiments refers to a constituent ring atom of the cyclic carrier, e.g., a carbon atom or a heteroatom (distinct from an atom which provides a backbone attachment point), that connects a selected moiety. The moiety can be, e.g., a carbohydrate, e.g. monosaccharide, disaccharide, trisaccharide, tetrasaccharide, oligosaccharide and polysaccharide. Optionally, the selected moiety is connected by an intervening tether to the cyclic carrier. Thus, the cyclic carrier will often include a functional group, e.g., an amino group, or generally, provide a bond, that is suitable for incorporation or tethering of another chemical entity, e.g., a ligand to the constituent ring.

The RNAi agents may be conjugated to a ligand via a carrier, wherein the carrier can be cyclic group or acyclic group; in some embodiments, the cyclic group is selected from pyrrolidinyl, pyrazolinyl, pyrazolidinyl, imidazolinyl, imidazolidinyl, piperidinyl, piperazinyl, [1,3]dioxolane, oxazolidinyl, isoxazolidinyl, morpholinyl, thiazolidinyl, isothiazolidinyl, quinoxalinyl, pyridazinonyl, tetrahydrofuryl and and decalin; in some embodiments, the acyclic group is selected from serinol backbone or diethanolamine backbone.

iii. Thermally Destabilizing Modifications

In certain embodiments, a nucleic acid inhibitor molecule can be optimized for RNA interference by incorporating thermally destabilizing modifications in the seed region of the antisense strand. As used herein “seed region” means at positions 2-9 of the 5′-end of the referenced strand. For example, thermally destabilizing modifications can be incorporated in the seed region of the antisense strand to reduce or inhibit off-target gene silencing.

The term “thermally destabilizing modification(s)” includes modification(s) that would result with a dsRNA with a lower overall melting temperature (Tₘ) than the Tₘ of the dsRNA without having such modification(s). For example, the thermally destabilizing modification(s) can decrease the Tₘ of the dsRNA by 1-4° C., such as one, two, three or four degrees Celcius. And, the term “thermally destabilizing nucleotide” refers to a nucleotide containing one or more thermally destabilizing modifications.

It has been discovered that dsRNAs with an antisense strand comprising at least one thermally destabilizing modification of the duplex within the first 9 nucleotide positions, counting from the 5′ end, of the antisense strand have reduced off-target gene silencing activity. Accordingly, in some embodiments, the antisense strand comprises at least one (e.g., one, two, three, four, five or more) thermally destabilizing modification of the duplex within the first 9 nucleotide positions of the 5′ region of the antisense strand. In some embodiments, one or more thermally destabilizing modification(s) of the duplex is/are located in positions 2-9, such as, positions 4-8, from the 5′-end of the antisense strand. In some further embodiments, the thermally destabilizing modification(s) of the duplex is/are located at position 6, 7 or 8 from the 5′-end of the antisense strand. In still some further embodiments, the thermally destabilizing modification of the duplex is located at position 7 from the 5′-end of the antisense strand. In some embodiments, the thermally destabilizing modification of the duplex is located at position 2, 3, 4, 5 or 9 from the 5′-end of the antisense strand.

In another embodiment of the invention, an iRNA agent comprises a sense strand and an antisense strand, each strand having 14 to 40 nucleotides. The RNAi agent may be represented by formula (L):

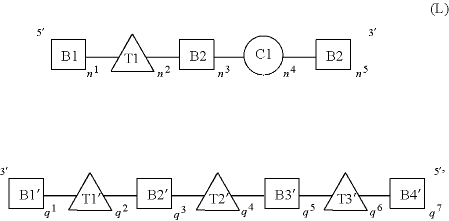

In formula (L), B1, B2, B3, B1′, B2′, B3′, and B4′ each are independently a nucleotide containing a modification selected from the group consisting of 2′-O-alkyl, 2′-substituted alkoxy, 2′-substituted alkyl, 2′-halo, ENA, and BNA/LNA. In one embodiment, B1, B2, B3, B1′, B2′, B3′, and B4′ each contain 2′-OMe modifications. In one embodiment, B1, B2, B3, B1′, B2′, B3′, and B4′ each contain 2′-OMe or 2′-F modifications. In one embodiment, at least one of B1, B2, B3, B1′, B2′, B3′, and B4′ contain 2′-O—N-methylacetamido (2′-O-NMA) modification.

C1 is a thermally destabilizing nucleotide placed at a site opposite to the seed region of the antisense strand (i.e., at positions 2-8 of the 5′-end of the antisense strand). For example, C1 is at a position of the sense strand that pairs with a nucleotide at positions 2-8 of the 5′-end of the antisense strand. In one example, C1 is at position 15 from the 5′-end of the sense strand. C1 nucleotide bears the thermally destabilizing modification which can include abasic modification; mismatch with the opposing nucleotide in the duplex; and sugar modification such as 2′-deoxy modification or acyclic nucleotide e.g., unlocked nucleic acids (UNA) or glycerol nucleic acid (GNA). In one embodiment, C1 has thermally destabilizing modification selected from the group consisting of: i) mismatch with the opposing nucleotide in the antisense strand; ii) abasic modification selected from the group consisting of:

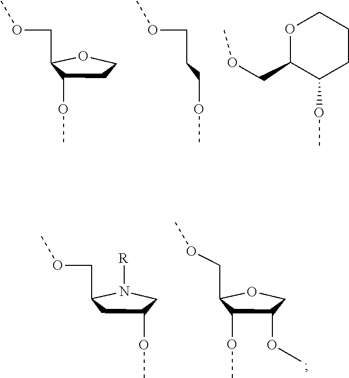

and iii) sugar modification selected from the group consisting of:

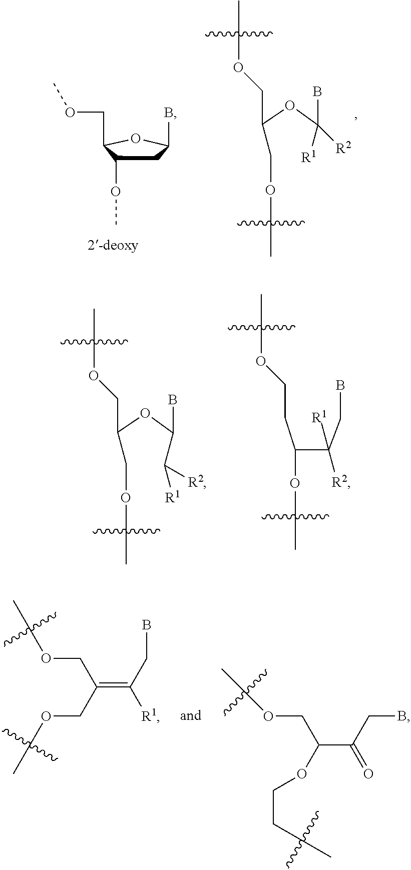

wherein B is a modified or unmodified nucleobase, R¹ and R² independently are H, halogen, OR₃, or alkyl; and R₃ is H, alkyl, cycloalkyl, aryl, aralkyl, heteroaryl or sugar. In one embodiment, the thermally destabilizing modification in C1 is a mismatch selected from the group consisting of G:G, G:A, G:U, G:T, A:A, A:C, C:C, C:U, C:T, U:U, T:T, and U:T; and optionally, at least one nucleobase in the mismatch pair is a 2′-deoxy nucleobase. In one example, the thermally destabilizing modification in C1 is GNA or

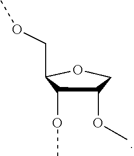

T1, T1′, T2′, and T3′ each independently represent a nucleotide comprising a modification providing the nucleotide a steric bulk that is less or equal to the steric bulk of a 2′-OMe modification. A steric bulk refers to the sum of steric effects of a modification. Methods for determining steric effects of a modification of a nucleotide are known to one skilled in the art. The modification can be at the 2′ position of a ribose sugar of the nucleotide, or a modification to a non-ribose nucleotide, acyclic nucleotide, or the backbone of the nucleotide that is similar or equivalent to the 2′ position of the ribose sugar, and provides the nucleotide a steric bulk that is less than or equal to the steric bulk of a 2′-OMe modification. For example, T1, T1′, T2′, and T3′ are each independently selected from DNA, RNA, LNA, 2′-F, and 2′-F-5′-methyl. In one embodiment, T1 is DNA. In one embodiment, T1′ is DNA, RNA or LNA. In one embodiment, T2′ is DNA or RNA. In one embodiment, T3′ is DNA or RNA.

n¹, n³, and q¹ are independently 4 to 15 nucleotides in length.

n⁵, q³, and q⁷ are independently 1-6 nucleotide(s) in length.

n⁴, q², and q⁶ are independently 1-3 nucleotide(s) in length; alternatively, n⁴ is 0.

q⁵ is independently 0-10 nucleotide(s) in length.

n² and q⁴ are independently 0-3 nucleotide(s) in length.

Alternatively, n⁴ is 0-3 nucleotide(s) in length.

In one embodiment, n⁴ can be 0. In one example, n⁴ is 0, and q² and q⁶ are 1. In another example, n⁴ is 0, and q² and q⁶ are 1, with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

In one embodiment, n⁴, q², and q⁶ are each 1.

In one embodiment, n², n⁴, q² q⁴, and q⁶ are each 1.

In one embodiment, C1 is at position 14-17 of the 5′-end of the sense strand, when the sense strand is 19-22 nucleotides in length, and n⁴ is 1. In one embodiment, C1 is at position 15 of the 5′-end of the sense strand

In one embodiment, T3′ starts at position 2 from the 5′ end of the antisense strand. In one example, T3′ is at position 2 from the 5′ end of the antisense strand and q⁶ is equal to 1.

In one embodiment, T1′ starts at position 14 from the 5′ end of the antisense strand. In one example, T1′ is at position 14 from the 5′ end of the antisense strand and q² is equal to 1.

In an exemplary embodiment, T3′ starts from position 2 from the 5′ end of the antisense strand and T1′ starts from position 14 from the 5′ end of the antisense strand. In one example, T3′ starts from position 2 from the 5′ end of the antisense strand and q⁶ is equal to 1 and T1′ starts from position 14 from the 5′ end of the antisense strand and q² is equal to 1.

In one embodiment, T1′ and T3′ are separated by 11 nucleotides in length (i.e. not counting the T1′ and T3′ nucleotides).

In one embodiment, T1′ is at position 14 from the 5′ end of the antisense strand. In one example, T1′ is at position 14 from the 5′ end of the antisense strand and q² is equal to 1, and the modification at the 2′ position or positions in a non-ribose, acyclic or backbone that provide less steric bulk than a 2′-OMe ribose.

In one embodiment, T3′ is at position 2 from the 5′ end of the antisense strand. In one example, T3′ is at position 2 from the 5′ end of the antisense strand and q⁶ is equal to 1, and the modification at the 2′ position or positions in a non-ribose, acyclic or backbone that provide less than or equal to steric bulk than a 2′-OMe ribose.

In one embodiment, T1 is at the cleavage site of the sense strand. In one example, T1 is at position 11 from the 5′ end of the sense strand, when the sense strand is 19-22 nucleotides in length, and n² is 1. In an exemplary embodiment, T1 is at the cleavage site of the sense strand at position 11 from the 5′ end of the sense strand, when the sense strand is 19-22 nucleotides in length, and n² is 1,

In one embodiment, T2′ starts at position 6 from the 5′ end of the antisense strand. In one example, T2′ is at positions 6-10 from the 5′ end of the antisense strand, and q⁴ is 1.

In an exemplary embodiment, T1 is at the cleavage site of the sense strand, for instance, at position 11 from the 5′ end of the sense strand, when the sense strand is 19-22 nucleotides in length, and n² is 1; T1′ is at position 14 from the 5′ end of the antisense strand, and q² is equal to 1, and the modification to T1′ is at the 2′ position of a ribose sugar or at positions in a non-ribose, acyclic or backbone that provide less steric bulk than a 2′-OMe ribose; T2′ is at positions 6-10 from the 5′ end of the antisense strand, and q⁴ is 1; and T3′ is at position 2 from the 5′ end of the antisense strand, and q⁶ is equal to 1, and the modification to T3′ is at the 2′ position or at positions in a non-ribose, acyclic or backbone that provide less than or equal to steric bulk than a 2′-OMe ribose.

In one embodiment, T2′ starts at position 8 from the 5′ end of the antisense strand. In one example, T2′ starts at position 8 from the 5′ end of the antisense strand, and q⁴ is 2.

In one embodiment, T2′ starts at position 9 from the 5′ end of the antisense strand. In one example, T2′ is at position 9 from the 5′ end of the antisense strand, and q⁴ is 1.

In one embodiment, B1′ is 2′-OMe or 2′-F, q¹ is 9, T1′ is 2′-F, q² is 1, B2′ is 2′-OMe or 2′-F, q³ is 4, T2′ is 2′-F, q⁴ is 1, B3′ is 2′-OMe or 2′-F, q⁵ is 6, T3′ is 2′-F, q⁶ is 1, B4′ is 2′-OMe, and q⁷ is 1; with two phosphorothioate internucleotide linkage modifications within positions 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

In one embodiment, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 1, B3′ is 2′-OMe or 2′-F, q⁵ is 6, T3′ is 2′-F, q⁶ is 1, B4′ is 2′-OMe, and q⁷ is 1; with two phosphorothioate internucleotide linkage modifications within positions 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within positions 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

In one embodiment, B1 is 2′-OMe or 2′-F, n¹ is 6, T1 is 2′F, n² is 3, B2 is 2′-OMe, n³ is 7, n⁴ is 0, B3 is 2′OMe, n⁵ is 3, B1′ is 2′-OMe or 2′-F, q¹ is 7, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1.

In one embodiment, B1 is 2′-OMe or 2′-F, n¹ is 6, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 7, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within positions 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 1, B3′ is 2′-OMe or 2′-F, q⁵ is 6, T3′ is 2′-F, q⁶ is 1, B4′ is 2′-OMe, and q⁷ is 1.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 1, B3′ is 2′-OMe or 2′-F, q⁵ is 6, T3′ is 2′-F, q⁶ is 1, B4′ is 2′-OMe, and q⁷ is 1; with two phosphorothioate internucleotide linkage modifications within positions 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 5, T2′ is 2′-F, q⁴ is 1, B3′ is 2′-OMe or 2′-F, q⁵ is 5, T3′ is 2′-F, q⁶ is 1, B4′ is 2′-OMe, and q⁷ is 1; optionally with at least 2 additional TT at the 3′-end of the antisense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 5, T2′ is 2′-F, q⁴ is 1, B3′ is 2′-OMe or 2′-F, q⁵ is 5, T3′ is 2′-F, q⁶ is 1, B4′ is 2′-OMe, and q⁷ is 1; optionally with at least 2 additional TT at the 3′-end of the antisense strand; with two phosphorothioate internucleotide linkage modifications within positions 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1; with two phosphorothioate internucleotide linkage modifications within positions 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end).
In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within positions 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within positions 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

The dsRNA agent can comprise a phosphorus-containing group at the 5′-end of the sense strand or antisense strand. The 5′-end phosphorus-containing group can be 5′-end phosphate (5′-P), 5′-end phosphorothioate (5′-PS), 5′-end phosphorodithioate (5′-PS₂), 5′-end vinylphosphonate (5′-VP), 5′-end methylphosphonate (MePhos), or 5′-deoxy-5′-C-malonyl

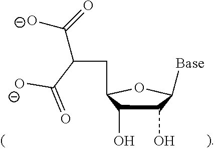

When the 5′-end phosphorus-containing group is 5′-end vinylphosphonate (5′-VP), the 5′-VP can be either 5′-E-VP isomer

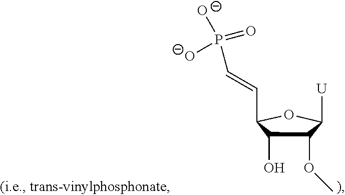

5′-Z-VP isomer

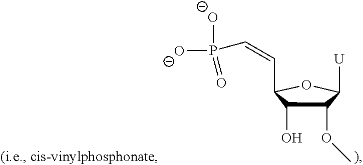

or mixtures thereof.

In one embodiment, the RNAi agent comprises a phosphorus-containing group at the 5′-end of the sense strand. In one embodiment, the RNAi agent comprises a phosphorus-containing group at the 5′-end of the antisense strand.

In one embodiment, the RNAi agent comprises a 5′-P. In one embodiment, the RNAi agent comprises a 5′-P in the antisense strand.

In one embodiment, the RNAi agent comprises a 5′-PS. In one embodiment, the RNAi agent comprises a 5′-PS in the antisense strand.

In one embodiment, the RNAi agent comprises a 5′-VP. In one embodiment, the RNAi agent comprises a 5′-VP in the antisense strand. In one embodiment, the RNAi agent comprises a 5′-E-VP in the antisense strand. In one embodiment, the RNAi agent comprises a 5′-Z-VP in the antisense strand.

In one embodiment, the RNAi agent comprises a 5′-PS₂. In one embodiment, the RNAi agent comprises a 5′-PS₂ in the antisense strand.

In one embodiment, the RNAi agent comprises a 5′-PS₂. In one embodiment, the RNAi agent comprises a 5′-deoxy-5′-C-malonyl in the antisense strand.

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1. The RNAi agent also comprises a 5′-PS.

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1. The RNAi agent also comprises a 5′-P.

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1. The RNAi agent also comprises a 5′-VP. The 5′-VP may be 5′-E-VP, 5′-Z-VP, or combination thereof.

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1. The RNAi agent also comprises a 5′-PS₂.

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1. The RNAi agent also comprises a 5′-deoxy-5′-C-malonyl.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-P.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-PS.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-VP. The 5′-VP may be 5′-E-VP, 5′-Z-VP, or combination thereof.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-PS₂.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-deoxy-5′-C-malonyl.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1. The RNAi agent also comprises a 5′-P.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1. The dsRNA agent also comprises a 5′-PS.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1. The RNAi agent also comprises a 5′-VP. The 5′-VP may be 5′-E-VP, 5′-Z-VP, or combination thereof.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1. The RNAi agent also comprises a 5′-PS₂.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1. The RNAi agent also comprises a 5′-deoxy-5′-C-malonyl.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The RNAi agent also comprises a 5′-P.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The RNAi agent also comprises a 5′-PS.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The RNAi agent also comprises a 5′-VP. The 5′-VP may be 5′-E-VP, 5′-Z-VP, or combination thereof.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The RNAi agent also comprises a 5′-PS₂.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The RNAi agent also comprises a 5′-deoxy-5′-C-malonyl.

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1. The RNAi agent also comprises a 5′-P.

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1. The RNAi agent also comprises a 5′-PS.

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1. The RNAi agent also comprises a 5′-VP. The 5′-VP may be 5′-E-VP, 5′-Z-VP, or combination thereof.

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1. The dsRNAi RNA agent also comprises a 5′-PS₂.

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1. The RNAi agent also comprises a 5′-deoxy-5′-C-malonyl.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-P.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-PS.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-VP. The 5′-VP may be 5′-E-VP, 5′-Z-VP, or combination thereof.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-PS₂.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-deoxy-5′-C-malonyl.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1. The RNAi agent also comprises a 5′-P.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1. The RNAi agent also comprises a 5′-PS.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1. The RNAi agent also comprises a 5′-VP. The 5′-VP may be 5′-E-VP, 5′-Z-VP, or combination thereof.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1. The RNAi agent also comprises a 5′-PS₂.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1. The RNAi agent also comprises a 5′-deoxy-5′-C-malonyl.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-P.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-PS.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-VP. The 5′-VP may be 5′-E-VP, 5′-Z-VP, or combination thereof.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-PS₂.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-deoxy-5′-C-malonyl.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-P and a targeting ligand. In one embodiment, the 5′-P is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-PS and a targeting ligand. In one embodiment, the 5′-PS is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-VP (e.g., a 5′-E-VP, 5′-Z-VP, or combination thereof), and a targeting ligand. In one embodiment, the 5′-VP is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-PS₂ and a targeting ligand. In one embodiment, the 5′-PS₂ is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-deoxy-5′-C-malonyl and a targeting ligand. In one embodiment, the 5′-deoxy-5′-C-malonyl is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The RNAi agent also comprises a 5′-P and a targeting ligand. In one embodiment, the 5′-P is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The RNAi agent also comprises a 5′-PS and a targeting ligand. In one embodiment, the 5′-PS is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The RNAi agent also comprises a 5′-VP (e.g., a 5′-E-VP, 5′-Z-VP, or combination thereof) and a targeting ligand. In one embodiment, the 5′-VP is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The RNAi agent also comprises a 5′-PS₂ and a targeting ligand. In one embodiment, the 5′-PS₂ is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The RNAi agent also comprises a 5′-deoxy-5′-C-malonyl and a targeting ligand. In one embodiment, the 5′-deoxy-5′-C-malonyl is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-P and a targeting ligand. In one embodiment, the 5′-P is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-PS and a targeting ligand. In one embodiment, the 5′-PS is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-VP (e.g., a 5′-E-VP, 5′-Z-VP, or combination thereof) and a targeting ligand. In one embodiment, the 5′-VP is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-PS₂ and a targeting ligand. In one embodiment, the 5′-PS₂ is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-deoxy-5′-C-malonyl and a targeting ligand. In one embodiment, the 5′-deoxy-5′-C-malonyl is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-P and a targeting ligand. In one embodiment, the 5′-P is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-PS and a targeting ligand. In one embodiment, the 5′-PS is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-VP (e.g., a 5′-E-VP, 5′-Z-VP, or combination thereof) and a targeting ligand. In one embodiment, the 5′-VP is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-PS₂ and a targeting ligand. In one embodiment, the 5′-PS₂ is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-deoxy-5′-C-malonyl and a targeting ligand. In one embodiment, the 5′-deoxy-5′-C-malonyl is at the 5′-end of the antisense strand, and the targeting ligand is at the 3′-end of the sense strand.

In a particular embodiment, an RNAi agent of the present invention comprises:
(a) a sense strand having:
   (i) a length of 21 nucleotides;
   (ii) an ASGPR ligand attached to the 3′-end, wherein said ASGPR ligand comprises three GalNAc derivatives attached through a trivalent branched linker; and
   (iii) 2′-F modifications at positions 1, 3, 5, 7, 9 to 11, 13, 17, 19, and 21, and 2′-OMe modifications at positions 2, 4, 6, 8, 12, 14 to 16, 18, and 20 (counting from the 5′ end);
   and
(b) an antisense strand having:
   (i) a length of 23 nucleotides;
   (ii) 2′-OMe modifications at positions 1, 3, 5, 9, 11 to 13, 15, 17, 19, 21, and 23, and 2′F modifications at positions 2, 4, 6 to 8, 10, 14, 16, 18, 20, and 22 (counting from the 5′ end); and
   (iii) phosphorothioate internucleotide linkages between nucleotide positions 21 and 22, and between nucleotide positions 22 and 23 (counting from the 5′ end);
   wherein the dsRNA agents have a two nucleotide overhang at the 3′-end of the antisense strand, and a blunt end at the 5′-end of the antisense strand.

In another particular embodiment, an RNAi agent of the present invention comprises:
(a) a sense strand having:
   (i) a length of 21 nucleotides;
   (ii) an ASGPR ligand attached to the 3′-end, wherein said ASGPR ligand comprises three GalNAc derivatives attached through a trivalent branched linker;
   (iii) 2′-F modifications at positions 1, 3, 5, 7, 9 to 11, 13, 15, 17, 19, and 21, and 2′-OMe modifications at positions 2, 4, 6, 8, 12, 14, 16, 18, and 20 (counting from the 5′ end); and
   (iv) phosphorothioate internucleotide linkages between nucleotide positions 1 and 2, and between nucleotide positions 2 and 3 (counting from the 5′ end);
   and
(b) an antisense strand having:
   (i) a length of 23 nucleotides;
   (ii) 2′-OMe modifications at positions 1, 3, 5, 7, 9, 11 to 13, 15, 17, 19, and 21 to 23, and 2′F modifications at positions 2, 4, 6, 8, 10, 14, 16, 18, and 20 (counting from the 5′ end); and
   (iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
wherein the RNAi agents have a two nucleotide overhang at the 3′-end of the antisense strand, and a blunt end at the 5′-end of the antisense strand.

In another particular embodiment, a RNAi agent of the present invention comprises:
(a) a sense strand having:
   (i) a length of 21 nucleotides;
   (ii) an ASGPR ligand attached to the 3′-end, wherein said ASGPR ligand comprises three GalNAc derivatives attached through a trivalent branched linker;
   (iii) 2′-OMe modifications at positions 1 to 6, 8, 10, and 12 to 21, 2′-F modifications at positions 7, and 9, and a desoxy-nucleotide (e.g. dT) at position 11 (counting from the 5′ end); and
   (iv) phosphorothioate internucleotide linkages between nucleotide positions 1 and 2, and between nucleotide positions 2 and 3 (counting from the 5′ end);
   and
(b) an antisense strand having:
   (i) a length of 23 nucleotides;
   (ii) 2′-OMe modifications at positions 1, 3, 7, 9, 11, 13, 15, 17, and 19 to 23, and 2′-F modifications at positions 2, 4 to 6, 8, 10, 12, 14, 16, and 18 (counting from the 5′ end); and
   (iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
wherein the RNAi agents have a two nucleotide overhang at the 3′-end of the antisense strand, and a blunt end at the 5′-end of the antisense strand.

In another particular embodiment, aRNAi agent of the present invention comprises:
(a) a sense strand having:
   (i) a length of 21 nucleotides;
   (ii) an ASGPR ligand attached to the 3′-end, wherein said ASGPR ligand comprises three GalNAc derivatives attached through a trivalent branched linker;
   (iii) 2′-OMe modifications at positions 1 to 6, 8, 10, 12, 14, and 16 to 21, and 2′-F modifications at positions 7, 9, 11, 13, and 15; and
   (iv) phosphorothioate internucleotide linkages between nucleotide positions 1 and 2, and between nucleotide positions 2 and 3 (counting from the 5′ end);
   and
(b) an antisense strand having:
   (i) a length of 23 nucleotides;
   (ii) 2′-OMe modifications at positions 1, 5, 7, 9, 11, 13, 15, 17, 19, and 21 to 23, and 2′-F modifications at positions 2 to 4, 6, 8, 10, 12, 14, 16, 18, and 20 (counting from the 5′ end); and
   (iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
wherein the RNAi agents have a two nucleotide overhang at the 3′-end of the antisense strand, and a blunt end at the 5′-end of the antisense strand.

In another particular embodiment, a RNAi agent of the present invention comprises:
(a) a sense strand having:
   (i) a length of 21 nucleotides;
   (ii) an ASGPR ligand attached to the 3′-end, wherein said ASGPR ligand comprises three GalNAc derivatives attached through a trivalent branched linker;
   (iii) 2′-OMe modifications at positions 1 to 9, and 12 to 21, and 2′-F modifications at positions 10, and 11; and
   (iv) phosphorothioate internucleotide linkages between nucleotide positions 1 and 2, and between nucleotide positions 2 and 3 (counting from the 5′ end);
   and
(b) an antisense strand having:
   (i) a length of 23 nucleotides;
   (ii) 2′-OMe modifications at positions 1, 3, 5, 7, 9, 11 to 13, 15, 17, 19, and 21 to 23, and 2′-F modifications at positions 2, 4, 6, 8, 10, 14, 16, 18, and 20 (counting from the 5′ end); and
   (iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
wherein the RNAi agents have a two nucleotide overhang at the 3′-end of the antisense strand, and a blunt end at the 5′-end of the antisense strand.

In another particular embodiment, a RNAi agent of the present invention comprises:
(a) a sense strand having:
   (i) a length of 21 nucleotides;
   (ii) an ASGPR ligand attached to the 3′-end, wherein said ASGPR ligand comprises three GalNAc derivatives attached through a trivalent branched linker;
   (iii) 2′-F modifications at positions 1, 3, 5, 7, 9 to 11, and 13, and 2′-OMe modifications at positions 2, 4, 6, 8, 12, and 14 to 21; and
   (iv) phosphorothioate internucleotide linkages between nucleotide positions 1 and 2, and between nucleotide positions 2 and 3 (counting from the 5′ end);
   and
(b) an antisense strand having:
   (i) a length of 23 nucleotides;
   (ii) 2′-OMe modifications at positions 1, 3, 5 to 7, 9, 11 to 13, 15, 17 to 19, and 21 to 23, and 2′-F modifications at positions 2, 4, 8, 10, 14, 16, and 20 (counting from the 5′ end); and
   (iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
wherein the RNAi agents have a two nucleotide overhang at the 3′-end of the antisense strand, and a blunt end at the 5′-end of the antisense strand.

In another particular embodiment, a RNAi agents of the present invention comprises:
(a) a sense strand having:
   (i) a length of 21 nucleotides;
   (ii) an ASGPR ligand attached to the 3′-end, wherein said ASGPR ligand comprises three GalNAc derivatives attached through a trivalent branched linker;
   (iii) 2′-OMe modifications at positions 1, 2, 4, 6, 8, 12, 14, 15, 17, and 19 to 21, and 2′-F modifications at positions 3, 5, 7, 9 to 11, 13, 16, and 18; and
   (iv) phosphorothioate internucleotide linkages between nucleotide positions 1 and 2, and between nucleotide positions 2 and 3 (counting from the 5′ end);
   and
(b) an antisense strand having:
   (i) a length of 25 nucleotides;
   (ii) 2′-OMe modifications at positions 1, 4, 6, 7, 9, 11 to 13, 15, 17, and 19 to 23, 2′-F modifications at positions 2, 3, 5, 8, 10, 14, 16, and 18, and desoxy-nucleotides (e.g. dT) at positions 24 and 25 (counting from the 5′ end); and
   (iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
wherein the RNAi agents have a four nucleotide overhang at the 3′-end of the antisense strand, and a blunt end at the 5′-end of the antisense strand.

In another particular embodiment, a RNAi agent of the present invention comprises:
(a) a sense strand having:
   (i) a length of 21 nucleotides;
   (ii) an ASGPR ligand attached to the 3′-end, wherein said ASGPR ligand comprises three GalNAc derivatives attached through a trivalent branched linker;
   (iii) 2′-OMe modifications at positions 1 to 6, 8, and 12 to 21, and 2′-F modifications at positions 7, and 9 to 11; and
   (iv) phosphorothioate internucleotide linkages between nucleotide positions 1 and 2, and between nucleotide positions 2 and 3 (counting from the 5′ end);
   and
(b) an antisense strand having:
   (i) a length of 23 nucleotides;
   (ii) 2′-OMe modifications at positions 1, 3 to 5, 7, 8, 10 to 13, 15, and 17 to 23, and 2′-F modifications at positions 2, 6, 9, 14, and 16 (counting from the 5′ end); and
   (iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
wherein the RNAi agents have a two nucleotide overhang at the 3′-end of the antisense strand, and a blunt end at the 5′-end of the antisense strand.

In another particular embodiment, a RNAi agent of the present invention comprises:
(a) a sense strand having:
   (i) a length of 21 nucleotides;
   (ii) an ASGPR ligand attached to the 3′-end, wherein said ASGPR ligand comprises three GalNAc derivatives attached through a trivalent branched linker;
   (iii) 2′-OMe modifications at positions 1 to 6, 8, and 12 to 21, and 2′-F modifications at positions 7, and 9 to 11; and
   (iv) phosphorothioate internucleotide linkages between nucleotide positions 1 and 2, and between nucleotide positions 2 and 3 (counting from the 5′ end);
   and
(b) an antisense strand having:
   (i) a length of 23 nucleotides;
   (ii) 2′-OMe modifications at positions 1, 3 to 5, 7, 10 to 13, 15, and 17 to 23, and 2′-F modifications at positions 2, 6, 8, 9, 14, and 16 (counting from the 5′ end); and
(iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
wherein the RNAi agents have a two nucleotide overhang at the 3′-end of the antisense strand, and a blunt end at the 5′-end of the antisense strand.

In another particular embodiment, a RNAi agent of the present invention comprises:
(a) a sense strand having:
   (i) a length of 19 nucleotides;
   (ii) an ASGPR ligand attached to the 3′-end, wherein said ASGPR ligand comprises three GalNAc derivatives attached through a trivalent branched linker;
   (iii) 2′-OMe modifications at positions 1 to 4, 6, and 10 to 19, and 2′-F modifications at positions 5, and 7 to 9; and
   (iv) phosphorothioate internucleotide linkages between nucleotide positions 1 and 2, and between nucleotide positions 2 and 3 (counting from the 5′ end);
   and
(b) an antisense strand having:
   (i) a length of 21 nucleotides;
   (ii) 2′-OMe modifications at positions 1, 3 to 5, 7, 10 to 13, 15, and 17 to 21, and 2′-F modifications at positions 2, 6, 8, 9, 14, and 16 (counting from the 5′ end); and
   (iii) phosphorothioate internucleotide linkages between nucleotide positions 1 and 2, between nucleotide positions 2 and 3, between nucleotide positions 19 and 20, and between nucleotide positions 20 and 21 (counting from the 5′ end);
wherein the RNAi agents have a two nucleotide overhang at the 3′-end of the antisense strand, and a blunt end at the 5′-end of the antisense strand.

### B. Single Stranded Antisense Polynucleotide Agents of the Invention

In one embodiment, a nucleic acid inhibitor for use in the methods of the invention is a single stranded antisense polynucleotide agent that targets LDHA, a single stranded antisense polynucleotide agent that targets PRODH2, and/or a single stranded antisense polynucleotide agent that targets HAO1.

Suitable antisense polynucleotide agent for use in the methods of the invention are known in the art and described in, for example, U.S. Patent Publication No. 2018/0092990 (Attorney Docket No. 121301-03602), the entire contents of which are incorporated herein by reference.

In certain specific embodiments, a nucleic acid inhibitor of the present invention is a single stranded antisense polynucleotide agent which inhibits the expression of an LDHA gene and is selected from the group of antisense sequence listed in any one of Tables 2-3. In some embodiments, a nucleic acid inhibitor of the present invention is a single stranded antisense polynucleotide agent which inhibits the expression of an HAO1 gene and is selected from the group of antisense sequence listed in any one of Tables 4-14. In some embodiments, a nucleic acid inhibitor of the present invention is a single stranded antisense polynucleotide agent which inhibits the expression of a PRODH2 gene and is selected from the group of antisense sequence listed in any one of Tables 15-16. Any of these agents may further comprise a ligand.

The polynucleotide agents of the invention include a nucleotide sequence which is about 4 to about 50 nucleotides or less in length and which is about 80% complementary to at least part of an mRNA transcript of an LDHA gene, a PRODH2 gene and/or HAO1 gene. The use of these polynucleotide agents enables the targeted inhibition of RNA expression and/or activity of a corresponding gene in subjects, such as human subjects.

The polynucleotide agents, e.g., antisense polynucleotide agents, and compositions comprising such agents, of the invention target an LDHA gene, a PRODH2 gene and/or an HAO1 gene and inhibit the expression of the gene. In one embodiment, the polynucleotide agents inhibit the expression of the gene in a cell, such as a cell within a subject, e.g., a mammal, such as a human having or at risk of developing a non-primary hyperoxaluria disease or disorder.

The polynucleotide agents of the invention include a region of complementarity which is complementary to at least a part of an mRNA formed in the expression of an LDHA gene, a PRODH2 gene and/or an HAO1 gene. The region of complementarity may be about 50 nucleotides or less in length (e.g., about 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, or 4 nucleotides or less in length). Upon contact with a cell expressing the gene, the polynucleotide agent inhibits the expression of the gene (e.g., a human, a primate, a non-primate, or a bird LDHA gene, PRODH2 gene and/or HAO1 gene) by at least about 10% as assayed by, for example, a PCR or branched DNA (bDNA)-based method, or by a protein-based method, such as by immunofluorescence analysis, using, for example, western Blotting or flow cytometric techniques.

The region of complementarity between a polynucleotide agent and a target sequence may be substantially complementary (e.g., there is a sufficient degree of complementarity between the polynucleotide agent and a target nucleic acid to so that they specifically hybridize and induce a desired effect), but is generally fully complementary to the target sequence. The target sequence can be derived from the sequence of an mRNA formed during the expression of an LDHA gene, a PRODH2 gene and/or an HAO1 gene.

In one aspect, an antisense polynucleotide agent, specifically hybridizes to a target nucleic acid molecule, such as the mRNA encoding LDHA, and comprises a contiguous nucleotide sequence which corresponds to the reverse complement of a nucleotide sequence of SEQ ID NOs:1, 3, 5, 7, or 9, or a fragment of SEQ ID NOs:1, 3, 5, 7, or 9.

In one aspect, an antisense polynucleotide agent, specifically hybridizes to a target nucleic acid molecule, such as the mRNA encoding HAO1, and comprises a contiguous nucleotide sequence which corresponds to the reverse complement of a nucleotide sequence of SEQ ID NO:21, or a fragment of SEQ ID NO:21.

In another aspect, an antisense polynucleotide agent, specifically hybridizes to a target nucleic acid molecule, such as the mRNA encoding PRODH2, and comprises a contiguous nucleotide sequence which corresponds to the reverse complement of a nucleotide sequence of SEQ ID NO:4641, or a fragment of SEQ ID NO:4641.

In some embodiments, the polynucleotide agents of the invention may be substantially complementary to the target sequence. For example, a polynucleotide agent that is substantially complementary to the target sequence may include a contiguous nucleotide sequence comprising no more than 5 mismatches (e.g., no more than 1, no more than 2, no more than 3, no more than 4, or no more than 5 mismatches) when hybridizing to a target sequence, such as to the corresponding region of a nucleic acid which encodes a mammalian LDHA mRNA, a mammalian PRODH2 mRNA, and/or a mammalian HAO1 mRNA. In some embodiments, the contiguous nucleotide sequence comprises no more than a single mismatch when hybridizing to the target sequence, such as the corresponding region of a nucleic acid which encodes a mammalian LDHA mRNA, a mammalian PRODH2 mRNA, and/or a mammalian HAO1 mRNA.

In some embodiments, the polynucleotide agents of the invention that are substantially complementary to the target sequence comprise a contiguous nucleotide sequence which is at least about 80% complementary over its entire length to the equivalent region of the nucleotide sequence of SEQ ID NOs:1, 3, 5, 7, or 9, or a fragment of SEQ ID NOs:1, 3, 5, 7, or 9, such as about 85%, about 86%, about 87%, about 88%, about 89%, about 90%, about % 91%, about 92%, about 93%, about 94%, about 95%, about 96%, about 97%, about 98%, or about 99% complementary.

In some embodiments, a polynucleotide agent comprises a contiguous nucleotide sequence which is fully complementary over its entire length to the equivalent region of the nucleotide sequence of SEQ ID NOs:1, 3, 5, 7, or 9 (or a fragment of SEQ ID NOs:1, 3, 5, 7, or 9).

In some embodiments, the polynucleotide agents of the invention that are substantially complementary to the target sequence comprise a contiguous nucleotide sequence which is at least about 80% complementary over its entire length to the equivalent region of the nucleotide sequence of SEQ ID NO:21, or a fragment of SEQ ID NO:21, such as about 85%, about 86%, about 87%, about 88%, about 89%, about 90%, about % 91%, about 92%, about 93%, about 94%, about 95%, about 96%, about 97%, about 98%, or about 99% complementary.

In some embodiments, a polynucleotide agent comprises a contiguous nucleotide sequence which is fully complementary over its entire length to the equivalent region of the nucleotide sequence of SEQ ID NO:21 (or a fragment of SEQ ID NO:21).

In some embodiments, the polynucleotide agents of the invention that are substantially complementary to the target sequence comprise a contiguous nucleotide sequence which is at least about 80% complementary over its entire length to the equivalent region of the nucleotide sequence of SEQ ID NO:4641, or a fragment of SEQ ID NO:4641, such as about 85%, about 86%, about 87%, about 88%, about 89%, about 90%, about % 91%, about 92%, about 93%, about 94%, about 95%, about 96%, about 97%, about 98%, or about 99% complementary.

In some embodiments, a polynucleotide agent comprises a contiguous nucleotide sequence which is fully complementary over its entire length to the equivalent region of the nucleotide sequence of SEQ ID NO:4641 (or a fragment of SEQ ID NO:4641).

A polynucleotide agent may comprise a contiguous nucleotide sequence of about 4 to about 50 nucleotides in length, e.g., 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length.

In some embodiments, a polynucleotide agent may comprise a contiguous nucleotide sequence of no more than 22 nucleotides, such as no more than 21 nucleotides, 20 nucleotides, 19 nucleotides, or no more than 18 nucleotides. In some embodiments the polynucleotide agents of the invention comprises less than 20 nucleotides. In other embodiments, the polynucleotide agents of the invention comprise 20 nucleotides.

In certain aspects, a polynucleotide agent of the invention targeting LDHA includes a sequence selected from the group of antisense sequences provided in any one of Tables 2-3.

In certain aspects, a polynucleotide agent of the invention targeting HAO1 includes a sequence selected from the group of antisense sequences provided many one of Tables 4-14.

In certain aspects, a polynucleotide agent of the invention targeting PRODH2 includes a sequence selected from the group of antisense sequences provided in any one of Tables 15-16.

It will be understood that, although some of the antisense sequences in Tables 2-16 are described as modified and/or conjugated sequences, a polynucleotide agent of the invention, may also comprise any one of the sequences set forth in Tables 2-16 that is un-modified, un-conjugated, and/or modified and/or conjugated differently than described therein.

By virtue of the nature of the nucleotide sequences provided in Tables 2-16, polynucleotide agents of the invention may include one of the sequences of Tables 2-16 minus only a few nucleotides on one or both ends and yet remain similarly effective as compared to the polynucleotide agents described above. Hence, polynucleotide agents having a sequence of at least 4, 5, 6, 7, 8, 9, 10, 11, 12, 13, 14, 15, 16, 17, 18, 19, 20, or more contiguous nucleotides derived from one of the sequences of Tables 2-14 and differing in their ability to inhibit the expression of the corresponding gene by not more than about 5, 10, 15, 20, 25, or 30% inhibition from an polynucleotide agent comprising the full sequence, are contemplated to be within the scope of the present invention.

In addition, the polynucleotide agents provided in Tables 2-16 identify a region(s) in an LDHA transcript, a PRODH2 transcript and/or an HAO1 transcript that is susceptible to antisense inhibition (e.g., the regions in SEQ ID NO: 1 or SEQ ID NO:21 or SEQ ID NO: 4641 which the polynucleotide agents may target). As such, the present invention further features polynucleotide agents that target within one of these sites. As used herein, a polynucleotide agent is said to target within a particular site of an RNA transcript if the polynucleotide agent promotes antisense inhibition of the target at that site. Such a polynucleotide agent will generally include at least about 15 contiguous nucleotides from one of the sequences provided in Tables 2-16 coupled to additional nucleotide sequences taken from the region contiguous to the selected sequence in the target gene.

While a target sequence is generally about 4-50 nucleotides in length, there is wide variation in the suitability of particular sequences in this range for directing antisense inhibition of any given target RNA. Various software packages and the guidelines set out herein provide guidance for the identification of optimal target sequences for any given gene target, but an empirical approach can also be taken in which a “window” or “mask” of a given size (as a non-limiting example, 20 nucleotides) is literally or figuratively (including, e.g., in silico) placed on the target RNA sequence to identify sequences in the size range that can serve as target sequences. By moving the sequence “window” progressively one nucleotide upstream or downstream of an initial target sequence location, the next potential target sequence can be identified, until the complete set of possible sequences is identified for any given target size selected. This process, coupled with systematic synthesis and testing of the identified sequences (using assays as described herein or as known in the art) to identify those sequences that perform optimally can identify those RNA sequences that, when targeted with a polynucleotide agent, mediate the best inhibition of target gene expression. Thus, while the sequences identified, for example, in Tables 2-16, represent effective target sequences, it is contemplated that further optimization of antisense inhibition efficiency can be achieved by progressively “walking the window” one nucleotide upstream or downstream of the given sequences to identify sequences with equal or better inhibition characteristics.

Further, it is contemplated that for any sequence identified, e.g., in Tables 2-16, further optimization could be achieved by systematically either adding or removing nucleotides to generate longer or shorter sequences and testing those sequences generated by walking a window of the longer or shorter size up or down the target RNA from that point. Again, coupling this approach to generating new candidate targets with testing for effectiveness of polynucleotide agents based on those target sequences in an inhibition assay as known in the art and/or as described herein can lead to further improvements in the efficiency of inhibition. Further still, such optimized sequences can be adjusted by, e.g., the introduction of modified nucleotides as described herein or as known in the art, addition or changes in length, or other modifications as known in the art and/or discussed herein to further optimize the molecule (e.g., increasing serum stability or circulating half-life, increasing thermal stability, enhancing transmembrane delivery, targeting to a particular location or cell type, increasing interaction with silencing pathway enzymes, increasing release from endosomes) as an expression inhibitor.

i. Single Stranded Polynucleotide Agents Comprising Motifs

In certain embodiments of the invention, at least one of the contiguous nucleotides of the antisense polynucleotide agents of the invention may be a modified nucleotide. Suitable nucleotide modifications for use in the single stranded antisense polynucleotide agents of the invention are described in Section A(ii), above. In one embodiment, the modified nucleotide comprises one or more modified sugars. In other embodiments, the modified nucleotide comprises one or more modified nucleobases. In yet other embodiments, the modified nucleotide comprises one or more modified internucleoside linkages. In some embodiments, the modifications (sugar modifications, nucleobase modifications, and/or linkage modifications) define a pattern or motif. In one embodiment, the patterns of modifications of sugar moieties, internucleoside linkages, and nucleobases are each independent of one another.

Polynucleotide agents having modified oligonucleotides arranged in patterns, or motifs may, for example, confer to the agents properties such as enhanced inhibitory activity, increased binding affinity for a target nucleic acid, or resistance to degradation by in vivo nucleases. For example, such agents may contain at least one region modified so as to confer increased resistance to nuclease degradation, increased cellular uptake, increased binding affinity for the target nucleic acid, and/or increased inhibitory activity. A second region of such agents may optionally serve as a substrate for the cellular endonuclease RNase H, which cleaves the RNA strand of an RNA:DNA duplex.

An exemplary polynucleotide agent having modified oligonucleotides arranged in patterns, or motifs is a gapmer. In a “gapmer”, an internal region or “gap” having a plurality of linked nucleotides that supports RNaseH cleavage is positioned between two external flanking regions or “wings” having a plurality of linked nucleotides that are chemically distinct from the linked nucleotides of the internal region. The gap segment generally serves as the substrate for endonuclease cleavage, while the wing segments comprise modified nucleotides.

The three regions of a gapmer motif (the 5′-wing, the gap, and the 3′-wing) form a contiguous sequence of nucleotides and may be described as “X-Y-Z”, wherein “X” represents the length of the 5-wing, “Y” represents the length of the gap, and “Z” represents the length of the 3′-wing. In one embodiment, a gapmer described as “X-Y-Z” has a configuration such that the gap segment is positioned immediately adjacent to each of the 5′ wing segment and the 3′ wing segment. Thus, no intervening nucleotides exist between the 5′ wing segment and gap segment, or the gap segment and the 3′ wing segment. Any of the compounds, e.g., antisense compounds, described herein can have a gapmer motif. In some embodiments, X and Z are the same, in other embodiments they are different.

In certain embodiments, the regions of a gapmer are differentiated by the types of modified nucleotides in the region. The types of modified nucleotides that may be used to differentiate the regions of a gapmer, in some embodiments, include β-D-ribonucleotides, β-D-deoxyribonucleotides, 2′-modified nucleotides, e.g., 2′-modified nucleotides (e.g., 2′-MOE, and 2′-O—CH3), and bicyclic sugar modified nucleotides (e.g., those having a 4′-(CH2)n-O-2′ bridge, where n=1 or n=2). In one embodiment, at least some of the modified nucleotides of each of the wings may differ from at least some of the modified nucleotides of the gap. For example, at least some of the modified nucleotides of each wing that are closest to the gap (the 3′-most nucleotide of the 5′-wing and the 5′-most nucleotide of the 3-wing) differ from the modified nucleotides of the neighboring gap nucleotides, thus defining the boundary between the wings and the gap. In certain embodiments, the modified nucleotides within the gap are the same as one another. In certain embodiments, the gap includes one or more modified nucleotides that differ from the modified nucleotides of one or more other nucleotides of the gap.

The length of the 5′-wing (X) of a gapmer may be 1 to 6 nucleotides in length, e.g., 2 to 6, 2 to 5, 3 to 6, 3 to 5, 1 to 5, 1 to 4, 1 to 3, 2 to 4 nucleotides in length, e.g., 1, 2, 3, 4, 5, or 6 nucleotides in length.

The length of the 3′-wing (Z) of a gapmer may be 1 to 6 nucleotides in length, e.g., 2 to 6, 2-5, 3 to 6, 3 to 5, 1 to 5, 1 to 4, 1 to 3, 2 to 4 nucleotides in length, e.g., 1, 2, 3, 4, 5, or 6 nucleotides in length.

The length of the gap (Y) of a gapmer may be 5 to 14 nucleotides in length, e.g., 5 to 13, 5 to 12, 5 to 11, 5 to 10, 5 to 9, 5 to 8, 5 to 7, 5 to 6, 6 to 14, 6 to 13, 6 to 12, 6 to 11, 6 to 10, 6 to 9, 6 to 8, 6 to 7, 7 to 14, 7 to 13, 7 to 12, 7 to 11, 7 to 10, 7 to 9, 7 to 8, 8 to 14, 8 to 13, 8 to 12, 8 to 11, 8 to 10, 8 to 9, 9 to 14, 9 to 13, 9 to 12, 9 to 11, 9 to 10, 10 to 14, 10 to 13, 10 to 12, 10 to 11, 11 to 14, 11 to 13, 11 to 12, 12 to 14, 12 to 13, or 13 to 14 nucleotides in length, e.g., 5, 6, 7, 8, 9, 10, 11, 12, 13, or 14 nucleotides in length.

In some embodiments of the invention X consists of 2, 3, 4, 5 or 6 nucleotides, Y consists of 7, 8, 9, 10, 11, or 12 nucleotides, and Z consists of 2, 3, 4, 5 or 6 nucleotides. Such gapmers include (X-Y-Z) 2-7-2, 2-7-3, 2-7-4, 2-7-5, 2-7-6, 3-7-2, 3-7-3, 3-7-4, 3-7-5, 3-7-6, 4-7-3, 4-7-4, 4-7-5, 4-7-6, 5-7-3, 5-7-4, 5-7-5, 5-7-6, 6-7-3, 6-7-4, 6-7-5, 6-7-6, 3-7-3, 3-7-4, 3-7-5, 3-7-6, 4-7-3, 4-7-4, 4-7-5, 4-7-6, 5-7-3, 5-7-4, 5-7-5, 5-7-6, 6-7-3, 6-7-4, 6-7-5, 6-7-6, 2-8-2, 2-8-3, 2-8-4, 2-8-5, 2-8-6, 3-8-2, 3-8-3, 3-8-4, 3-8-5, 3-8-6, 4-8-3, 4-8-4, 4-8-5, 4-8-6, 5-8-3, 5-8-4, 5-8-5, 5-8-6, 6-8-3, 6-8-4, 6-8-5, 6-8-6, 2-9-2, 2-9-3, 2-9-4, 2-9-5, 2-9-6, 3-9-2, 3-9-3, 3-9-4, 3-9-5, 3-9-6, 4-9-3, 4-9-4, 4-9-5, 4-9-6, 5-9-3, 5-9-4, 5-9-5, 5-9-6, 6-9-3, 6-9-4, 6-9-5, 6-9-6, 2-10-2, 2-10-3, 2-10-4, 2-10-5, 2-10-6, 3-10-2, 3-10-3, 3-10-4, 3-10-5, 3-10-6, 4-10-3, 4-10-4, 4-10-5, 4-10-6, 5-10-3, 5-10-4, 5-10-5, 5-10-6, 6-10-3, 6-10-4, 6-10-5, 6-10-6, 2-11-2, 2-11-3, 2-11-4, 2-11-5, 2-11-6, 3-11-2, 3-11-3, 3-11-4, 3-11-5, 3-11-6, 4-11-3, 4-11-4, 4-11-5, 4-11-6, 5-11-3, 5-11-4, 5-11-5, 5-11-6, 6-11-3, 6-11-4, 6-11-5, 6-11-6, 2-12-2, 2-12-3, 2-12-4, 2-12-5, 2-12-6, 3-12-2, 3-12-3, 3-12-4, 3-12-5, 3-12-6, 4-12-3, 4-12-4, 4-12-5, 4-12-6, 5-12-3, 5-12-4, 5-12-5, 5-12-6, 6-12-3, 6-12-4, 6-12-5, or 6-12-6.

In some embodiments of the invention, polynucleotide agents of the invention include a 5-10-5 gapmer motif. In other embodiments of the invention, polynucleotide agents of the invention include a 4-10-4 gapmer motif. In another embodiment of the invention, polynucleotide agents of the invention include a 3-10-3 gapmer motif. In yet other embodiments of the invention, polynucleotide agents of the invention include a 2-10-2 gapmer motif.

The 5′-wing and/or 3′-wing of a gapmer may independently include 1-6 modified nucleotides, e.g., 1, 2, 3, 4, 5, or 6 modified nucleotides.

In some embodiment, the 5′-wing of a gapmer includes at least one modified nucleotide. In one embodiment, the 5′-wing of a gapmer comprises at least two modified nucleotides. In another embodiment, the 5′-wing of a gapmer comprises at least three modified nucleotides. In yet another embodiment, the 5′-wing of a gapmer comprises at least four modified nucleotides. In another embodiment, the 5′-wing of a gapmer comprises at least five modified nucleotides. In certain embodiments, each nucleotide of the 5′-wing of a gapmer is a modified nucleotide.

In some embodiments, the 3′-wing of a gapmer includes at least one modified nucleotide. In one embodiment, the 3′-wing of a gapmer comprises at least two modified nucleotides. In another embodiment, the 3′-wing of a gapmer comprises at least three modified nucleotides. In yet another embodiment, the 3′-wing of a gapmer comprises at least four modified nucleotides. In another embodiment, the 3′-wing of a gapmer comprises at least five modified nucleotides. In certain embodiments, each nucleotide of the 3′-wing of a gapmer is a modified nucleotide.

In certain embodiments, the regions of a gapmer are differentiated by the types of sugar moieties of the nucleotides. In one embodiment, the nucleotides of each distinct region comprise uniform sugar moieties. In other embodiments, the nucleotides of each distinct region comprise different sugar moieties. In certain embodiments, the sugar nucleotide modification motifs of the two wings are the same as one another. In certain embodiments, the sugar nucleotide modification motifs of the 5′-wing differs from the sugar nucleotide modification motif of the 3′-wing.

The 5′-wing of a gapmer may include 1-6 modified nucleotides, e.g., 1, 2, 3, 4, 5, or 6 modified nucleotides.

In one embodiment, at least one modified nucleotide of the 5′-wing of a gapmer is a bicyclic nucleotide, such as a constrained ethyl nucleotide, or an LNA. In another embodiment, the 5′-wing of a gapmer includes 2, 3, 4, or 5 bicyclic nucleotides. In some embodiments, each nucleotide of the 5′-wing of a gapmer is a bicyclic nucleotide.

In one embodiment, the 5′-wing of a gapmer includes at least 1, 2, 3, 4, or 5 constrained ethyl nucleotides. In some embodiments, each nucleotide of the 5′-wing of a gapmer is a constrained ethyl nucleotide.

In one embodiment, the 5′-wing of a gapmer comprises at least one LNA nucleotide. In another embodiment, the 5′-wing of a gapmer includes 2, 3, 4, or 5 LNA nucleotides. In other embodiments, each nucleotide of the 5′-wing of a gapmer is an LNA nucleotide.

In certain embodiments, at least one modified nucleotide of the 5′-wing of a gapmer is a non-bicyclic modified nucleotide, e.g., a 2′-substituted nucleotide. A “2′-substituted nucleotide” is a nucleotide comprising a modification at the 2′-position which is other than H or OH, such as a 2′-OMe nucleotide, or a 2′-MOE nucleotide. In one embodiment, the 5′-wing of a gapmer comprises 2, 3, 4, or 5 2′-substituted nucleotides. In one embodiment, each nucleotide of the 5′-wing of a gapmer is a 2′-substituted nucleotide.

In one embodiment, the 5′-wing of a gapmer comprises at least one 2′-OMe nucleotide. In one embodiment, the 5′-wing of a gapmer comprises at least 2, 3, 4, or 5 2′-OMe nucleotides. In one embodiment, each of the nucleotides of the 5′-wing of a gapmer comprises a 2′-OMe nucleotide. In one embodiment, the 5′-wing of a gapmer comprises at least one 2′-MOE nucleotide. In one embodiment, the 5′-wing of a gapmer comprises at least 2, 3, 4, or 5 2′-MOE nucleotides. In one embodiment, each of the nucleotides of the 5′-wing of a gapmer comprises a 2′-MOE nucleotide. In certain embodiments, the 5′-wing of a gapmer comprises at least one 2′-deoxynucleotide. In certain embodiments, each nucleotide of the 5′-wing of a gapmer is a 2′-deoxynucleotide. In a certain embodiments, the 5′-wing of a gapmer comprises at least one ribonucleotide. In certain embodiments, each nucleotide of the 5′-wing of a gapmer is a ribonucleotide.

The 3′-wing of a gapmer may include 1-6 modified nucleotides, e.g., 1, 2, 3, 4, 5, or 6 modified nucleotides.

In one embodiment, at least one modified nucleotide of the 3′-wing of a gapmer is a bicyclic nucleotide, such as a constrained ethyl nucleotide, or an LNA. In another embodiment, the 3′-wing of a gapmer includes 2, 3, 4, or 5 bicyclic nucleotides. In some embodiments, each nucleotide of the 3′-wing of a gapmer is a bicyclic nucleotide.

In one embodiment, the 3′-wing of a gapmer includes at least one constrained ethyl nucleotide. In another embodiment, the 3′-wing of a gapmer includes 2, 3, 4, or 5 constrained ethyl nucleotides. In some embodiments, each nucleotide of the 3′-wing of a gapmer is a constrained ethyl nucleotide.

In one embodiment, the 3′-wing of a gapmer comprises at least one LNA nucleotide. In another embodiment, the 3′-wing of a gapmer includes 2, 3, 4, or 5 LNA nucleotides. In other embodiments, each nucleotide of the 3′-wing of a gapmer is an LNA nucleotide.

In certain embodiments, at least one modified nucleotide of the 3′-wing of a gapmer is a non-bicyclic modified nucleotide, e.g., a 2′-substituted nucleotide. In one embodiment, the 3′-wing of a gapmer comprises 2, 3, 4, or 5 2′-substituted nucleotides. In one embodiment, each nucleotide of the 3′-wing of a gapmer is a 2′-substituted nucleotide.

In one embodiment, the 3′-wing of a gapmer comprises at least one 2′-OMe nucleotide. In one embodiment, the 3′-wing of a gapmer comprises at least 2, 3, 4, or 5 2′-OMe nucleotides. In one embodiment, each of the nucleotides of the 3′-wing of a gapmer comprises a 2′-OMe nucleotide.

In one embodiment, the 3′-wing of a gapmer comprises at least one 2′-MOE nucleotide. In one embodiment, the 3′-wing of a gapmer comprises at least 2, 3, 4, or 5 2′-MOE nucleotides. In one embodiment, each of the nucleotides of the 3′-wing of a gapmer comprises a 2′-MOE nucleotide. In certain embodiments, the 3′-wing of a gapmer comprises at least one 2′-deoxynucleotide. In certain embodiments, each nucleotide of the 3′-wing of a gapmer is a 2′-deoxynucleotide. In a certain embodiments, the 3′-wing of a gapmer comprises at least one ribonucleotide. In certain embodiments, each nucleotide of the 3′-wing of a gapmer is a ribonucleotide.

The gap of a gapmer may include 5-14 modified nucleotides, e.g., 5, 6, 7, 8, 9, 10, 11, 12, 13, or 14 modified nucleotides.

In one embodiment, the gap of a gapmer comprises at least one 5-methylcytosine. In one embodiment, the gap of a gapmer comprises at least 2, 3, 4, 5, 6, 7, 8, 9, 10, 11, 12, or 13 5-methylcytosines. In one embodiment, all of the nucleotides of the the gap of a gapmer are 5-methylcytosines.

In one embodiment, the gap of a gapmer comprises at least one 2′-deoxynucleotide. In one embodiment, the gap of a gapmer comprises at least 2, 3, 4, 5, 6, 7, 8, 9, 10, 11, 12, or 13 2′-deoxynucleotides. In one embodiment, all of the nucleotides of the the gap of a gapmer are 2′-deoxynucleotides.

A gapmer may include one or more modified internucleotide linkages. In some embodiments, a gapmer includes one or more phosphodiester internucleotide linkages. In other embodiments, a gapmer includes one or more phosphorothioate internucleotide linkages.

In one embodiment, each nucleotide of a 5′-wing of a gapmer are linked via a phosphorothioate internucleotide linkage. In another embodiment, each nucleotide of a 3′-wing of a gapmer are linked via a phosphorothioate internucleotide linkage. In yet another embodiment, each nucleotide of a gap segment of a gapmer is linked via a phosphorothioate internucleotide linkage. In one embodiment, all of the nucleotides in a gapmer are linked via phosphorothioate internucleotide linkages.

In one embodiment, a polynucleotide agent comprises a gap segment of ten 2′-deoxyribonucleotides positioned immediately adjacent to and between a 5′-wing segment comprising five nucleotides and a 3′-wing segment comprising 5 nucleotides.

In another embodiment, a polynucleotide agent comprises a gap segment of ten 2′-deoxyribonucleotides positioned immediately adjacent to and between a 5′-wing segment comprising four nucleotides and a 3′-wing segment comprising four nucleotides.

In another embodiment, a polynucleotide agent comprises a gap segment of ten 2′-deoxyribonucleotides positioned immediately adjacent to and between a 5′-wing segment comprising three nucleotides and a 3′-wing segment comprising three nucleotides.

In another embodiment, a polynucleotide agent comprises a gap segment of ten 2′-deoxyribonucleotides positioned immediately adjacent to and between a 5′-wing segment comprising two nucleotides and a 3′-wing segment comprising two nucleotides.

In one embodiment, each nucleotide of a 5-wing flanking a gap segment of 10 2′-deoxyribonucleotides comprises a modified nucleotide. In another embodiment, each nucleotide of a 3-wing flanking a gap segment of 10 2′-deoxyribonucleotides comprises a modified nucleotide. In one embodiment, each of the modified 5′-wing nucleotides and each of the modified 3′-wing nucleotides comprise a 2′-sugar modification. In one embodiment, the 2′-sugar modification is a 2′-OMe modification. In another embodiment, the 2′-sugar modification is a 2′-MOE modification. In one embodiment, each of the modified 5′-wing nucleotides and each of the modified 3′-wing nucleotides comprise a bicyclic nucleotide. In one embodiment, the bicyclic nucleotide is a constrained ethyl nucleotide. In another embodiment, the bicyclic nucleotide is an LNA nucleotide.

In one embodiment, each cytosine in a polynucleotide agent is a 5-methylcytosine.

In one embodiment, a polynucleotide agent comprises a gap segment of ten 2′-deoxyribonucleotides positioned immediately adjacent to and between a 5′-wing segment comprising five nucleotides comprising a 2′OMe modification and a 3′-wing segment comprising five nucleotides comprising a 2′OMe modification, wherein each internucleotide linkage of the agent is a phosphorothioate linkage. In one embodiment, each cytosine of the agent is a 5-methylcytosine. In one embodiment, the agent further comprises a ligand.

In one embodiment, a polynucleotide agent of the invention comprises a gap segment of ten 2′-deoxyribonucleotides positioned immediately adjacent to and between a 5′-wing segment comprising five nucleotides comprising a 2′MOE modification and a 3′-wing segment comprising five nucleotides comprising a 2′MOE modification, wherein each internucleotide linkage of the agent is a phosphorothioate linkage. In one embodiment, each cytosine of the agent is a 5-methylcytosine. In one embodiment, the agent further comprises a ligand.

In one embodiment, a polynucleotide agent of the invention comprises a gap segment of ten 2′-deoxyribonucleotides positioned immediately adjacent to and between a 5′-wing segment comprising five constrained ethyl nucleotides and a 3′-wing segment comprising five constrained ethyl nucleotides, wherein each internucleotide linkage of the agent is a phosphorothioate linkage. In one embodiment, each cytosine of the agent is a 5-methylcytosine.

In one embodiment, a polynucleotide agent of the invention comprises a gap segment of ten 2′-deoxyribonucleotides positioned immediately adjacent to and between a 5′-wing segment comprising five LNA nucleotides and a 3′-wing segment comprising five LNA nucleotides, wherein each internucleotide linkage of the agent is a phosphorothioate linkage. In one embodiment, each cytosine of the agent is a 5-methylcytosine.

In one embodiment, a polynucleotide agent of the invention comprises a gap segment of ten 2′-deoxyribonucleotides positioned immediately adjacent to and between a 5′-wing segment comprising four nucleotides comprising a 2′OMe modification and a 3′-wing segment comprising four nucleotides comprising a 2′OMe modification, wherein each internucleotide linkage of the agent is a phosphorothioate linkage. In one embodiment, each cytosine of the agent is a 5-methylcytosine. In one embodiment, a polynucleotide agent tof the invention comprises a gap segment of ten 2′-deoxyribonucleotides positioned immediately adjacent to and between a 5′-wing segment comprising four nucleotides comprising a 2′MOE modification and a 3′-wing segment comprising four nucleotides comprising a 2′MOE modification, wherein each internucleotide linkage of the agent is a phosphorothioate linkage. In one embodiment, each cytosine of the agent is a 5-methylcytosine.

In one embodiment, a polynucleotide agent of the invention comprises a gap segment of ten 2′-deoxyribonucleotides positioned immediately adjacent to and between a 5′-wing segment comprising four constrained ethyl nucleotides and a 3′-wing segment comprising four constrained ethyl nucleotides, wherein each internucleotide linkage of the agent is a phosphorothioate linkage. In one embodiment, each cytosine of the agent is a 5-methylcytosine.

In one embodiment, a polynucleotide agent of the invention comprises a gap segment of ten 2′-deoxyribonucleotides positioned immediately adjacent to and between a 5′-wing segment comprising four LNA nucleotides and a 3′-wing segment comprising four LNA nucleotides, wherein each internucleotide linkage of the agent is a phosphorothioate linkage. In one embodiment, each cytosine of the agent is a 5-methylcytosine.

In one embodiment, a polynucleotide agent of the invention comprises a gap segment of ten 2′-deoxyribonucleotides positioned immediately adjacent to and between a 5′-wing segment comprising three nucleotides comprising a 2′OMe modification and a 3′-wing segment comprising three nucleotides comprising a 2′OMe modification, wherein each internucleotide linkage of the agent is a phosphorothioate linkage. In one embodiment, each cytosine of the agent is a 5-methylcytosine.

In one embodiment, a polynucleotide agent of the invention comprises a gap segment of ten 2′-deoxyribonucleotides positioned immediately adjacent to and between a 5′-wing segment comprising three nucleotides comprising a 2′MOE modification and a 3′-wing segment comprising three nucleotides comprising a 2′MOE modification, wherein each internucleotide linkage of the agent is a phosphorothioate linkage. In one embodiment, each cytosine of the agent is a 5-methylcytosine.

In one embodiment, a polynucleotide agent of the invention comprises a gap segment of ten 2′-deoxyribonucleotides positioned immediately adjacent to and between a 5′-wing segment comprising three constrained ethyl nucleotides and a 3′-wing segment comprising three constrained ethyl nucleotides, wherein each internucleotide linkage of the agent is a phosphorothioate linkage. In one embodiment, each cytosine of the agent is a 5-methylcytosine.

In one embodiment, a polynucleotide agent of the invention comprises a gap segment of ten 2′-deoxyribonucleotides positioned immediately adjacent to and between a 5′-wing segment comprising three LNA nucleotides and a 3′-wing segment comprising three LNA nucleotides, wherein each internucleotide linkage of the agent is a phosphorothioate linkage. In one embodiment, each cytosine of the agent is a 5-methylcytosine.

In one embodiment, a polynucleotide agent of the invention comprises a gap segment of ten 2′-deoxyribonucleotides positioned immediately adjacent to and between a 5′-wing segment comprising two nucleotides comprising a 2′OMe modification and a 3′-wing segment comprising two nucleotides comprising a 2′OMe modification, wherein each internucleotide linkage of the agent is a phosphorothioate linkage. In one embodiment, each cytosine of the agent is a 5-methylcytosine.

In one embodiment, a polynucleotide agent of the invention comprises a gap segment of ten 2′-deoxyribonucleotides positioned immediately adjacent to and between a 5′-wing segment comprising two nucleotides comprising a 2′MOE modification and a 3′-wing segment comprising two nucleotides comprising a 2′MOE modification, wherein each internucleotide linkage of the agent is a phosphorothioate linkage. In one embodiment, each cytosine of the agent is a 5-methylcytosine.

In one embodiment, a polynucleotide agent of the invention comprises a gap segment of ten 2′-deoxyribonucleotides positioned immediately adjacent to and between a 5′-wing segment comprising two constrained ethyl nucleotides and a 3′-wing segment comprising two constrained ethyl nucleotides, wherein each internucleotide linkage of the agent is a phosphorothioate linkage. In one embodiment, each cytosine of the agent is a 5-methylcytosine.

In one embodiment, a polynucleotide agent of the invention comprises a gap segment of ten 2′-deoxyribonucleotides positioned immediately adjacent to and between a 5′-wing segment comprising two LNA nucleotides and a 3′-wing segment comprising two LNA nucleotides, wherein each internucleotide linkage of the agent is a phosphorothioate linkage. In one embodiment, each cytosine of the agent is a 5-methylcytosine.

Further gapmer designs suitable for use in the agents, compositions, and methods of the invention are disclosed in, for example, U.S. Pat. Nos. 7,687,617 and 8,580,756; U.S. Patent Publication Nos. 20060128646, 20090209748, 20140128586, 20140128591, 20100210712, and 20080015162A1; and International Publication No. WO 2013/159108, the entire content of each of which are incorporated herein by reference.

### C. Nucleic Acid Inhibitors Conjugated to Ligands

Another modification of a nucleic acid inhibitor of the invention involves chemically linking to the nucleic acid inhibitor one or more ligands, moieties or conjugates that enhance the activity, cellular distribution or cellular uptake of the nucleic acid inhibitor. Such moieties include but are not limited to lipid moieties such as a cholesterol moiety (Letsinger et al., (1989) Proc. Natl. Acid. Sci. USA, 86: 6553-6556), cholic acid (Manoharan et al., (1994) Biorg. Med. Chem. Let., 4:1053-1060), a thioether, e.g., beryl-S-tritylthiol (Manoharan et al., (1992) Ann. N.Y. Acad. Sci., 660:306-309; Manoharan et al., (1993) Biorg. Med. Chem. Let., 3:2765-2770), a thiocholesterol (Oberhauser et al., (1992) Nucl. Acids Res., 20:533-538), an aliphatic chain, e.g., dodecandiol or undecyl residues (Saison-Behmoaras et al., (1991) EMBO J, 10:1111-1118; Kabanov et al., (1990) FEBS Lett., 259:327-330; Svinarchuk et al., (1993) Biochimie, 75:49-54), a phospholipid, e.g., di-hexadecyl-rac-glycerol or triethyl-ammonium 1,2-di-O-hexadecyl-rac-glycero-3-phosphonate (Manoharan et al., (1995) Tetrahedron Lett., 36:3651-3654; Shea et al., (1990) Nucl. Acids Res., 18:3777-3783), a polyamine or a polyethylene glycol chain (Manoharan et al., (1995) Nucleosides & Nucleotides, 14:969-973), or adamantane acetic acid (Manoharan et al., (1995) Tetrahedron Lett., 36:3651-3654), a palmityl moiety (Mishra et al., (1995) Biochim. Biophys. Acta, 1264:229-237), or an octadecylamine or hexylamino-carbonyloxycholesterol moiety (Crooke et al., (1996) J. Pharmacol. Exp. Ther., 277:923-937).

In embodiments in which a first dsRNA agent targeting LDHA and a second dsRNA agent targeting HAO1 are covalently attached (i.e., a dual targeting RNAi agent described herein), one or both of the dsRNA agents may independently comprise one or more ligands.

In one embodiment, a ligand alters the distribution, targeting or lifetime of a nucleic acid inhibitor into which it is incorporated. In certain embodiments a ligand provides an enhanced affinity for a selected target, e.g., molecule, cell or cell type, compartment, e.g., a cellular or organ compartment, tissue, organ or region of the body, as, e.g., compared to a species absent such a ligand. In some embodiments, ligands will not take part in duplex pairing in a duplexed nucleic acid.

Ligands can include a naturally occurring substance, such as a protein (e.g., human serum albumin (HSA), low-density lipoprotein (LDL), or globulin); carbohydrate (e.g., a dextran, pullulan, chitin, chitosan, inulin, cyclodextrin, N-acetylglucosamine, N-acetylgalactosamine or hyaluronic acid); or a lipid. The ligand can also be a recombinant or synthetic molecule, such as a synthetic polymer, e.g., a synthetic polyamino acid. Examples of polyamino acids include polyamino acid is a polylysine (PLL), poly L-aspartic acid, poly L-glutamic acid, styrene-maleic acid anhydride copolymer, poly(L-lactide-co-glycolide) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacryllic acid), N-isopropylacrylamide polymers, or polyphosphazine. Example of polyamines include: polyethylenimine, polylysine (PLL), spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, or an alpha helical peptide.

Ligands can also include targeting groups, e.g., a cell or tissue targeting agent, e.g., a lectin, glycoprotein, lipid or protein, e.g., an antibody, that binds to a specified cell type such as a kidney cell. A targeting group can be a thyrotropin, melanotropin, lectin, glycoprotein, surfactant protein A, Mucin carbohydrate, multivalent lactose, multivalent galactose, N-acetyl-galactosamine, N-acetyl-glucosamine multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, vitamin A, biotin, or an RGD peptide or RGD peptide mimetic.

Other examples of ligands include dyes, intercalating agents (e.g. acridines), cross-linkers (e.g. psoralene, mitomycin C), porphyrins (TPPC4, texaphyrin, Sapphyrin), polycyclic aromatic hydrocarbons (e.g., phenazine, dihydrophenazine), artificial endonucleases (e.g. EDTA), lipophilic molecules, e.g., cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid,O3-(oleoyl)lithocholic acid, O3-(oleoyl)cholenic acid, dimethoxytrityl, or phenoxazine) and peptide conjugates (e.g., antennapedia peptide, Tat peptide), alkylating agents, phosphate, amino, mercapto, PEG (e.g., PEG-40K), MPEG, [MPEG]₂, polyamino, alkyl, substituted alkyl, radiolabeled markers, enzymes, haptens (e.g. biotin), transport/absorption facilitators (e.g., aspirin, vitamin E, folic acid), synthetic ribonucleases (e.g., imidazole, bisimidazole, histamine, imidazole clusters, acridine-imidazole conjugates, Eu3+ complexes of tetraazamacrocycles), dinitrophenyl, HRP, or AP.

Ligands can be proteins, e.g., glycoproteins, or peptides, e.g., molecules having a specific affinity for a co-ligand, or antibodies e.g., an antibody, that binds to a specified cell type such as a hepatic cell. Ligands can also include hormones and hormone receptors. They can also include non-peptidic species, such as lipids, lectins, carbohydrates, vitamins, cofactors, multivalent lactose, multivalent galactose, N-acetyl-galactosamine, N-acetyl-glucosamine multivalent mannose, or multivalent fucose. The ligand can be, for example, a lipopolysaccharide, an activator of p38 MAP kinase, or an activator of NF-κB.

The ligand can be a substance, e.g., a drug, which can increase the uptake of the nucleic acid inhibitor into the cell, for example, by disrupting the cell's cytoskeleton, e.g., by disrupting the cell's microtubules, microfilaments, and/or intermediate filaments. The drug can be, for example, taxon, vincristine, vinblastine, cytochalasin, nocodazole, japlakinolide, latrunculin A, phalloidin, swinholide A, indanocine, or myoservin.

In some embodiments, a ligand attached to a nucleic acid inhibitor as described herein acts as a pharmacokinetic modulator (PK modulator). PK modulators include lipophiles, bile acids, steroids, phospholipid analogues, peptides, protein binding agents, PEG, vitamins etc. Exemplary PK modulators include, but are not limited to, cholesterol, fatty acids, cholic acid, lithocholic acid, dialkylglycerides, diacylglyceride, phospholipids, sphingolipids, naproxen, ibuprofen, vitamin E, biotin etc. Oligonucleotides that comprise a number of phosphorothioate linkages are also known to bind to serum protein, thus short oligonucleotides, e.g., oligonucleotides of about 5 bases, 10 bases, 15 bases or 20 bases, comprising multiple of phosphorothioate linkages in the backbone are also amenable to the present invention as ligands (e.g. as PK modulating ligands). In addition, aptamers that bind serum components (e.g. serum proteins) are also suitable for use as PK modulating ligands in the embodiments described herein.

Ligand-conjugated nucleic acid inhibitors of the invention may be synthesized by the use of an oligonucleotide that bears a pendant reactive functionality, such as that derived from the attachment of a linking molecule onto the oligonucleotide (described below). This reactive oligonucleotide may be reacted directly with commercially-available ligands, ligands that are synthesized bearing any of a variety of protecting groups, or ligands that have a linking moiety attached thereto.

The oligonucleotides used in the conjugates of the present invention may be conveniently and routinely made through the well-known technique of solid-phase synthesis. Equipment for such synthesis is sold by several vendors including, for example, Applied Biosystems (Foster City, Calif.). Any other means for such synthesis known in the art may additionally or alternatively be employed. It is also known to use similar techniques to prepare other oligonucleotides, such as the phosphorothioates and alkylated derivatives.

In the ligand-conjugated oligonucleotides and ligand-molecule bearing sequence-specific linked nucleosides of the present invention, the oligonucleotides and oligonucleosides may be assembled on a suitable DNA synthesizer utilizing standard nucleotide or nucleoside precursors, or nucleotide or nucleoside conjugate precursors that already bear the linking moiety, ligand-nucleotide or nucleoside-conjugate precursors that already bear the ligand molecule, or non-nucleoside ligand-bearing building blocks.

When using nucleotide-conjugate precursors that already bear a linking moiety, the synthesis of the sequence-specific linked nucleosides is typically completed, and the ligand molecule is then reacted with the linking moiety to form the ligand-conjugated oligonucleotide. In some embodiments, the oligonucleotides or linked nucleosides of the present invention are synthesized by an automated synthesizer using phosphoramidites derived from ligand-nucleoside conjugates in addition to the standard phosphoramidites and non-standard phosphoramidites that are commercially available and routinely used in oligonucleotide synthesis.

i. Lipid Conjugates

In one embodiment, the ligand or conjugate is a lipid or lipid-based molecule. In one embodiment, such a lipid or lipid-based molecule binds a serum protein, e.g., human serum albumin (HSA). An HSA binding ligand allows for distribution of the conjugate to a target tissue, e.g., a non-kidney target tissue of the body. For example, the target tissue can be the liver, including parenchymal cells of the liver. Other molecules that can bind HSA can also be used as ligands. For example, neproxin or aspirin can be used. A lipid or lipid-based ligand can (a) increase resistance to degradation of the conjugate, (b) increase targeting or transport into a target cell or cell membrane, and/or (c) can be used to adjust binding to a serum protein, e.g., HSA.

A lipid based ligand can be used to inhibit, e.g., control the binding of the conjugate to a target tissue. For example, a lipid or lipid-based ligand that binds to HSA more strongly will be less likely to be targeted to the kidney and therefore less likely to be cleared from the body. A lipid or lipid-based ligand that binds to HSA less strongly can be used to target the conjugate to the kidney.

In one embodiment, the lipid based ligand binds HSA. For example, it binds HSA with a sufficient affinity such that the conjugate will be distributed to a non-kidney tissue. However, it is preferred that the affinity not be so strong that the HSA-ligand binding cannot be reversed.

In another embodiment, the lipid based ligand binds HSA weakly or not at all, such that the conjugate will be distributed to the kidney. Other moieties that target to kidney cells can also be used in place of or in addition to the lipid based ligand.

In another aspect, the ligand is a moiety, e.g., a vitamin, which is taken up by a target cell, e.g., a proliferating cell. These are particularly useful for treating disorders characterized by unwanted cell proliferation, e.g., of the malignant or non-malignant type, e.g., cancer cells. Exemplary vitamins include vitamin A, E, and K. Other exemplary vitamins include are B vitamin, e.g., folic acid, B12, riboflavin, biotin, pyridoxal or other vitamins or nutrients taken up by target cells such as liver cells. Also included are HSA and low density lipoprotein (LDL).

ii. Cell Permeation Agents

In another aspect, the ligand is a cell-permeation agent, such as, a helical cell-permeation agent. In one embodiment, the agent is amphipathic. An exemplary agent is a peptide such as tat or antennopedia. If the agent is a peptide, it can be modified, including a peptidylmimetic, invertomers, non-peptide or pseudo-peptide linkages, and use of D-amino acids. In some embodiments, the helical agent is an alpha-helical agent, which may have a lipophilic and a lipophobic phase.

The ligand can be a peptide or peptidomimetic. A peptidomimetic (also referred to herein as an oligopeptidomimetic) is a molecule capable of folding into a defined three-dimensional structure similar to a natural peptide. The attachment of peptide and peptidomimetics to nucleic acid inhibitors can affect pharmacokinetic distribution of the nucleic acid inhibitor, such as by enhancing cellular recognition and absorption. The peptide or peptidomimetic moiety can be about 5-50 amino acids long, e.g., about 5, 10, 15, 20, 25, 30, 35, 40, 45, or 50 amino acids long.

A peptide or peptidomimetic can be, for example, a cell permeation peptide, cationic peptide, amphipathic peptide, or hydrophobic peptide (e.g., consisting primarily of Tyr, Trp or Phe). The peptide moiety can be a dendrimer peptide, constrained peptide or crosslinked peptide. In another alternative, the peptide moiety can include a hydrophobic membrane translocation sequence (MTS). An exemplary hydrophobic MTS-containing peptide is RFGF having the amino acid sequence AAVALLPAVLLALLAP (SEQ ID NO: 4154). An RFGF analogue (e.g., amino acid sequence AALLPVLLAAP (SEQ ID NO: 4151) containing a hydrophobic MTS can also be a targeting moiety. The peptide moiety can be a “delivery” peptide, which can carry large polar molecules including peptides, oligonucleotides, and protein across cell membranes. For example, sequences from the HIV Tat protein (GRKKRRQRRRPPQ (SEQ ID NO: 4152) and the Drosophila Antennapedia protein (RQIKIWFQNRRMKWKK (SEQ ID NO: 4153) have been found to be capable of functioning as delivery peptides. A peptide or peptidomimetic can be encoded by a random sequence of DNA, such as a peptide identified from a phage-display library, or one-bead-one-compound (OBOC) combinatorial library (Lam et al., Nature, 354:82-84, 1991). Examples of a peptide or peptidomimetic tethered to a nucleic acid inhibitor via an incorporated monomer unit for cell targeting purposes is an arginine-glycine-aspartic acid (RGD)-peptide, or RGD mimic. A peptide moiety can range in length from about 5 amino acids to about 40 amino acids. The peptide moieties can have a structural modification, such as to increase stability or direct conformational properties. Any of the structural modifications described below can be utilized.

An RGD peptide for use in the compositions and methods of the invention may be linear or cyclic, and may be modified, e.g., glycosylated or methylated, to facilitate targeting to a specific tissue(s). RGD-containing peptides and peptidomimetics may include D-amino acids, as well as synthetic RGD mimics. In addition to RGD, one can use other moieties that target the integrin ligand. Exemplary conjugates of this ligand target PECAM-1 or VEGF.

A “cell permeation peptide” is capable of permeating a cell, e.g., a microbial cell, such as a bacterial or fungal cell, or a mammalian cell, such as a human cell. A microbial cell-permeating peptide can be, for example, a α-helical linear peptide (e.g., LL-37 or Ceropin P1), a disulfide bond-containing peptide (e.g., α-defensin, β-defensin or bactenecin), or a peptide containing only one or two dominating amino acids (e.g., PR-39 or indolicidin). A cell permeation peptide can also include a nuclear localization signal (NLS). For example, a cell permeation peptide can be a bipartite amphipathic peptide, such as MPG, which is derived from the fusion peptide domain of HIV-1 gp41 and the NLS of SV40 large T antigen (Simeoni et al., Nucl. Acids Res. 31:2717-2724, 2003).

iii. Carbohydrate Conjugates

In some embodiments of the compositions and methods of the invention, a nucleic acid inhibitor further comprises a carbohydrate. The carbohydrate conjugated nucleic acid inhibitors are advantageous for the in vivo delivery of nucleic acids, as well as compositions suitable for in vivo therapeutic use, as described herein. As used herein, “carbohydrate” refers to a compound which is either a carbohydrate per se made up of one or more monosaccharide units having at least 6 carbon atoms (which can be linear, branched or cyclic) with an oxygen, nitrogen or sulfur atom bonded to each carbon atom; or a compound having as a part thereof a carbohydrate moiety made up of one or more monosaccharide units each having at least six carbon atoms (which can be linear, branched or cyclic), with an oxygen, nitrogen or sulfur atom bonded to each carbon atom. Representative carbohydrates include the sugars (mono-, di-, tri- and oligosaccharides containing from about 4, 5, 6, 7, 8, or 9 monosaccharide units), and polysaccharides such as starches, glycogen, cellulose and polysaccharide gums. Specific monosaccharides include C5 and above (e.g., C5, C6, C7, or C8) sugars; di- and trisaccharides include sugars having two or three monosaccharide units (e.g., C5, C6, C7, or C8).

In embodiments in which a first dsRNA agent targeting LDHA and a second dsRNA agent targeting HAO1 are covalently attached (i.e., a dual targeting RNAi agent), one or both of the dsRNA agents may independently comprise one or more carbohydrate ligands.

In one embodiment, a carbohydrate conjugate for use in the compositions and In certain embodiments, a carbohydrate conjugate comprises a monosaccharide.

In certain embodiments, the monosaccharide is an N-acetylgalactosamine (GalNAc). GalNAc conjugates, which comprise one or more N-acetylgalactosamine (GalNAc) derivatives, are described, for example, in U.S. Pat. No. 8,106,022, the entire content of which is hereby incorporated herein by reference. In some embodiments, the GalNAc conjugate serves as a ligand that targets the nucleic acid inhibitor to particular cells. In some embodiments, the GalNAc conjugate targets the nucleic acid inhibitor to liver cells, e.g., by serving as a ligand for the asialoglycoprotein receptor of liver cells (e.g., hepatocytes).

In some embodiments, the carbohydrate conjugate comprises one or more GalNAc derivatives. The GalNAc derivatives may be attached via a linker, e.g., a bivalent or trivalent branched linker. In some embodiments the GalNAc conjugate is conjugated to the 3′ end of the sense strand. In some embodiments, the GalNAc conjugate is conjugated to the nucleic acid inhibitor (e.g., to the 3′ end of the sense strand) via a linker, e.g., a linker as described herein. In some embodiments the GalNAc conjugate is conjugated to the 5′ end of the sense strand. In some embodiments, the GalNAc conjugate is conjugated to the nucleic acid inhibitor (e.g., to the 5′ end of the sense strand) via a linker, e.g., a linker as described herein.

In certain embodiments of the invention, the GalNAc or GalNAc derivative is attached to a nucleic acid inhibitor of the invention via a monovalent linker. In some embodiments, the GalNAc or GalNAc derivative is attached to a nucleic acid inhibitor of the invention via a bivalent linker. In yet other embodiments of the invention, the GalNAc or GalNAc derivative is attached to a nucleic acid inhibitor of the invention via a trivalent linker. In other embodiments of the invention, the GalNAc or GalNAc derivative is attached to a nucleic acid inhibitor of the invention via a tetravalent linker.

In certain embodiments, the nucleic acid inhibitors of the invention comprise one GalNAc or GalNAc derivative attached to the nucleic acid inhibitor. In certain embodiments, the nucleic acid inhibitors of the invention comprise a plurality (e.g., 2, 3, 4, 5, or 6) GalNAc or GalNAc derivatives, each independently attached to a plurality of nucleotides of the nucleic acid inhibitor through a plurality of monovalent linkers.

In some embodiments, for example, when two strands of a nucleic acid inhibitor of the invention are part of one larger molecule connected by an uninterrupted chain of nucleotides between the 3′-end of one strand and the 5′-end of the respective other strand forming a hairpin loop comprising, a plurality of unpaired nucleotides, each unpaired nucleotide within the hairpin loop may independently comprise a GalNAc or GalNAc derivative attached via a monovalent linker. The hairpin loop may also be formed by an extended overhang in one strand of the duplex.

In some embodiments, for example, when the two strands of a nucleic acid inhibitor of the invention are part of one larger molecule connected by an uninterrupted chain of nucleotides between the 3′-end of one strand and the 5′-end of the respective other strand forming a hairpin loop comprising, a plurality of unpaired nucleotides, each unpaired nucleotide within the hairpin loop may independently comprise a GalNAc or GalNAc derivative attached via a monovalent linker. The hairpin loop may also be formed by an extended overhang in one strand of the duplex.

In some embodiments, the GalNAc conjugate is

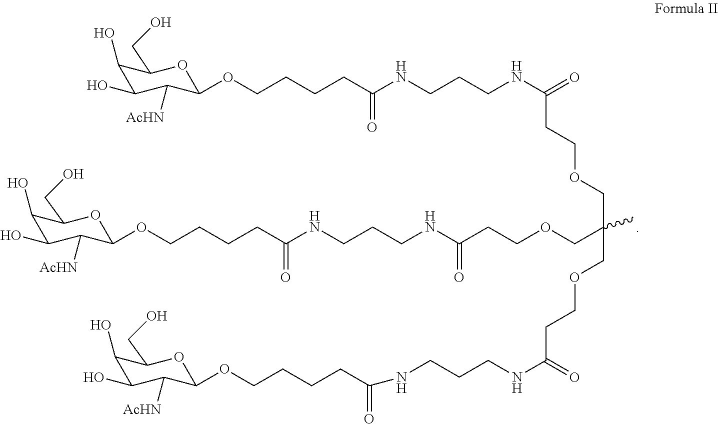 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

In some embodiments, the RNAi agent is attached to the carbohydrate conjugate via a linker as shown in the following schematic, wherein X is O or S

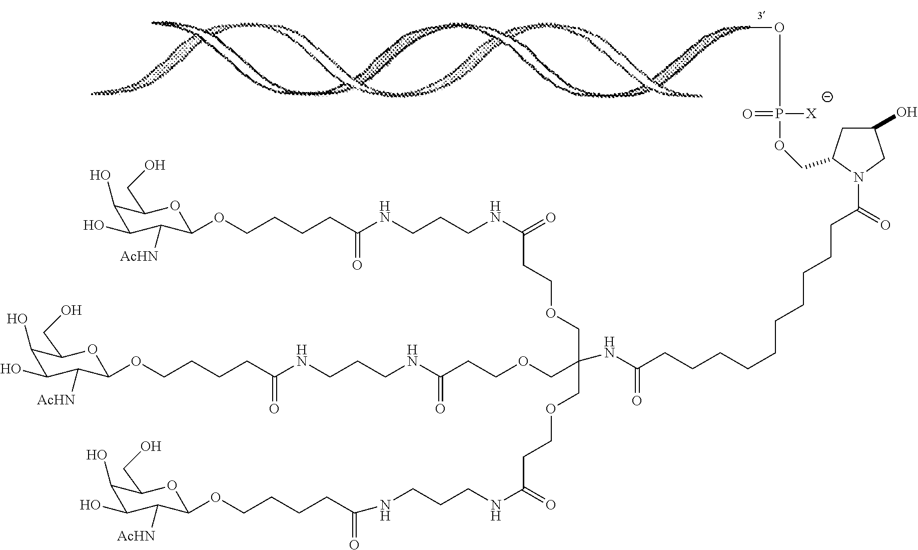

In some embodiments, the RNAi agent is conjugated to L96 as defined in Table 1 and shown below:

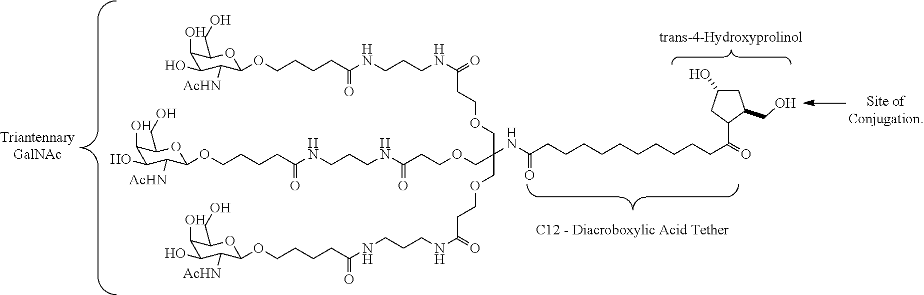

In certain embodiments, a carbohydrate conjugate for use in the compositions and methods of the invention is selected from the group consisting of:

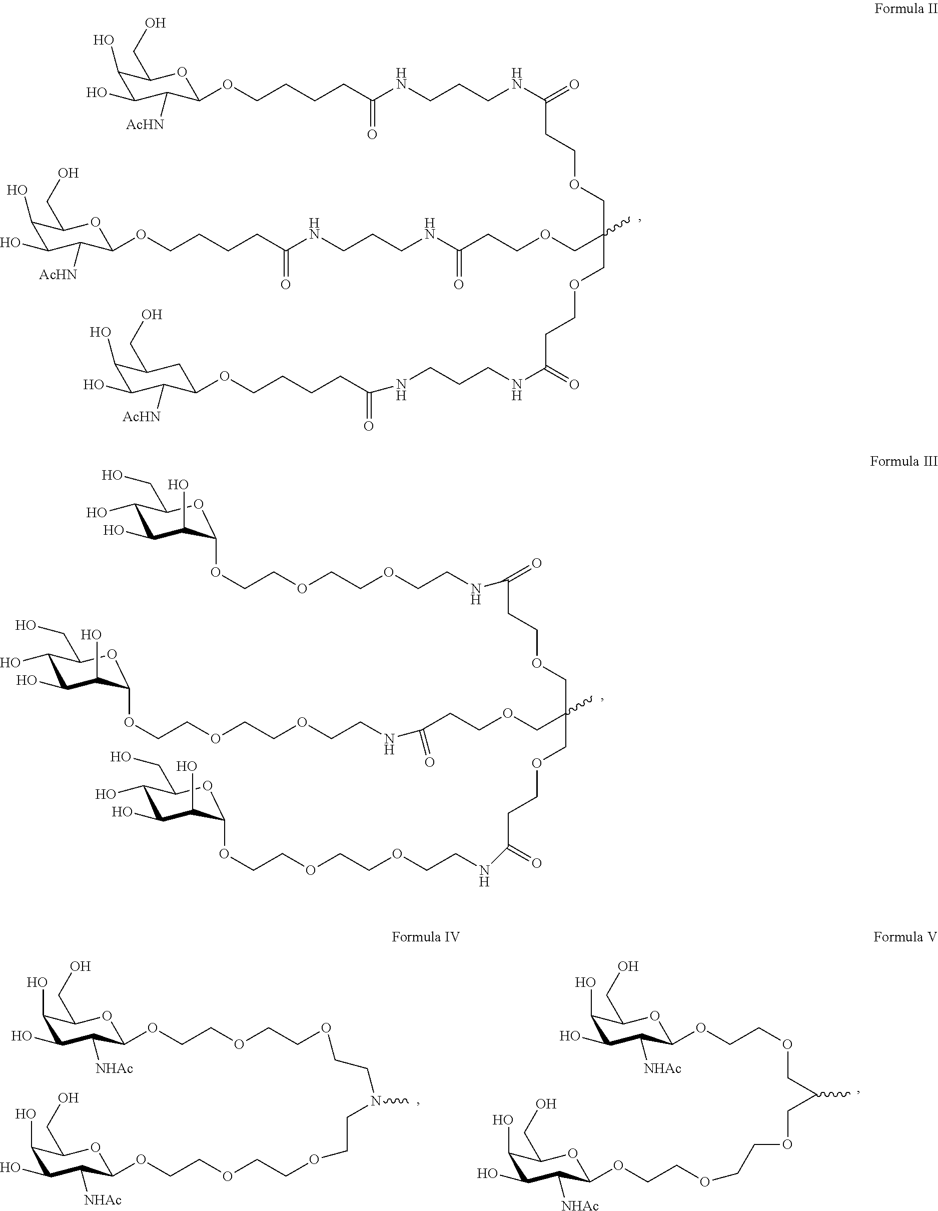

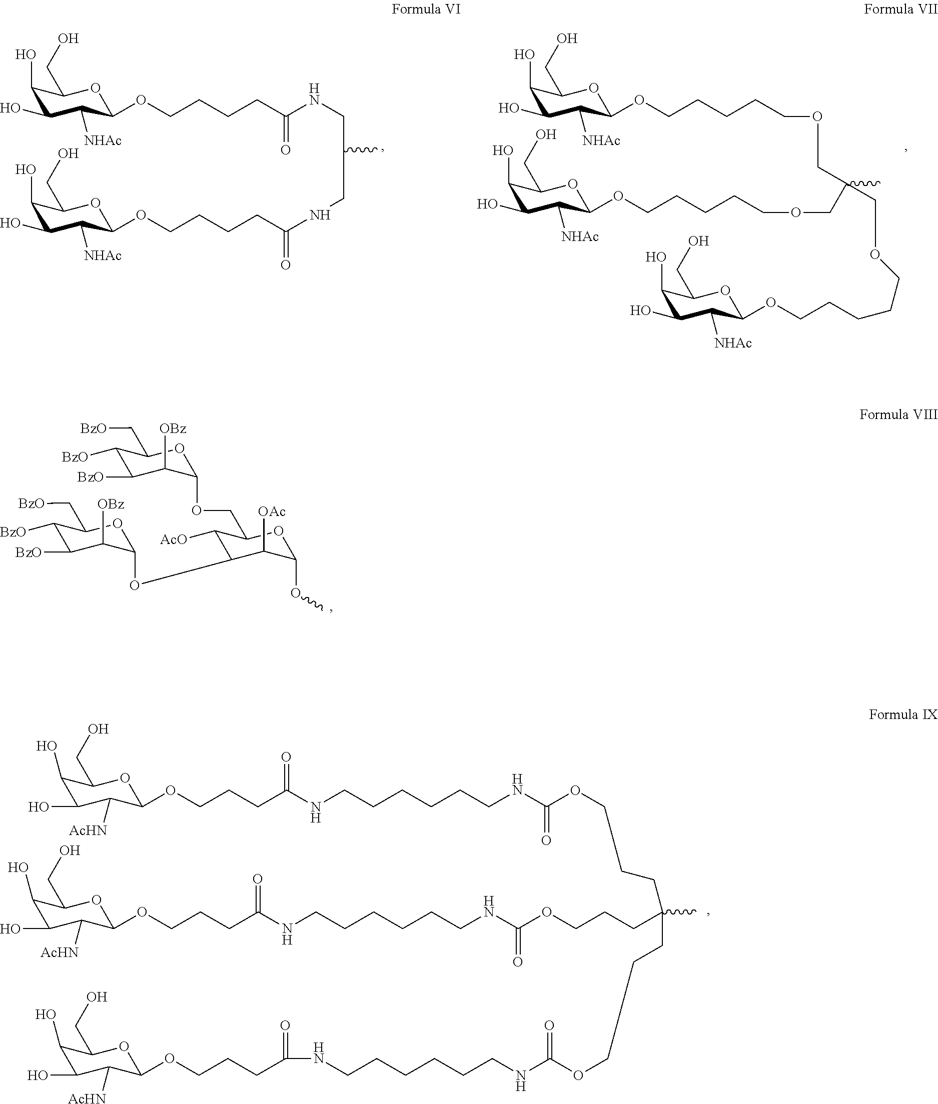

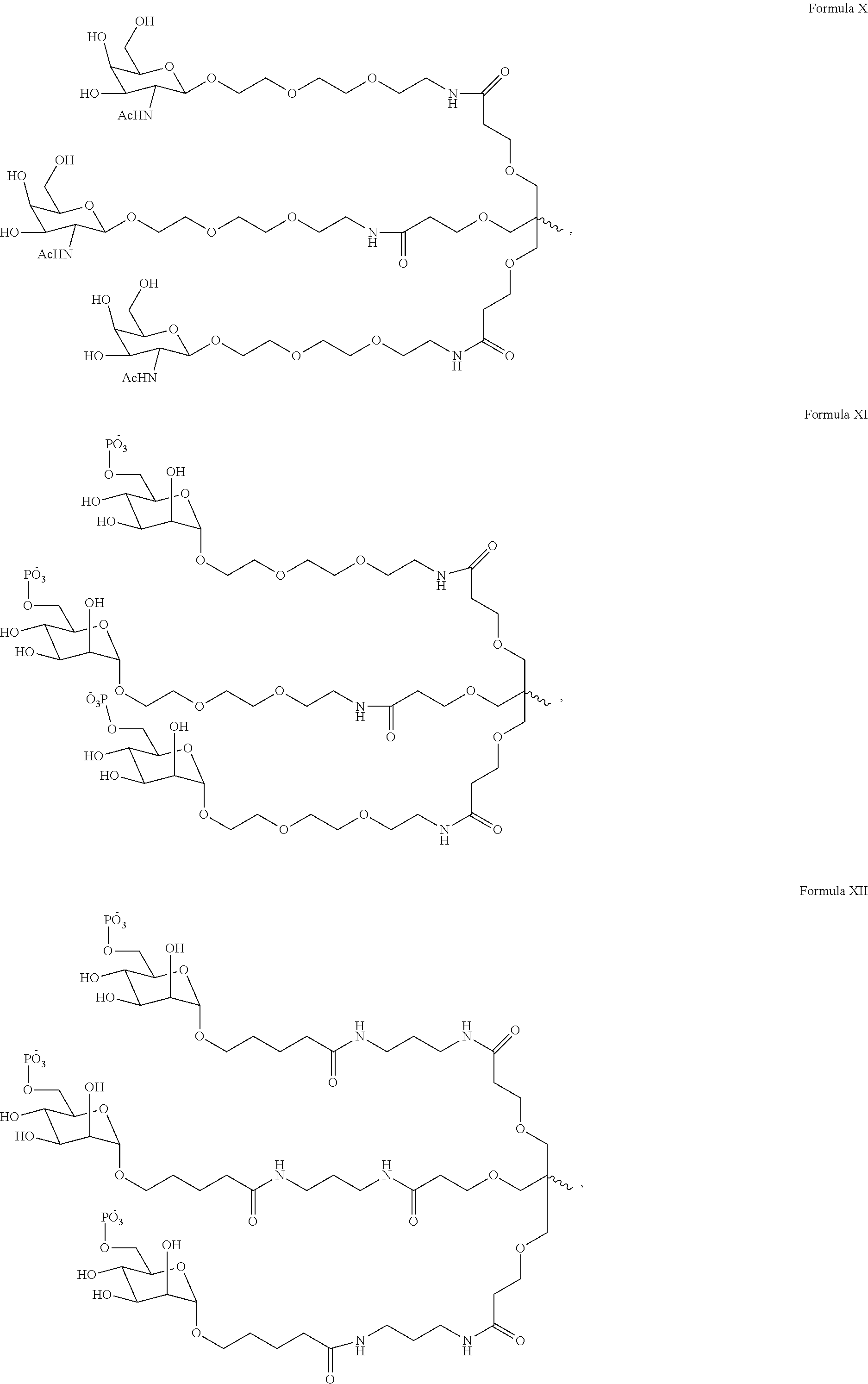

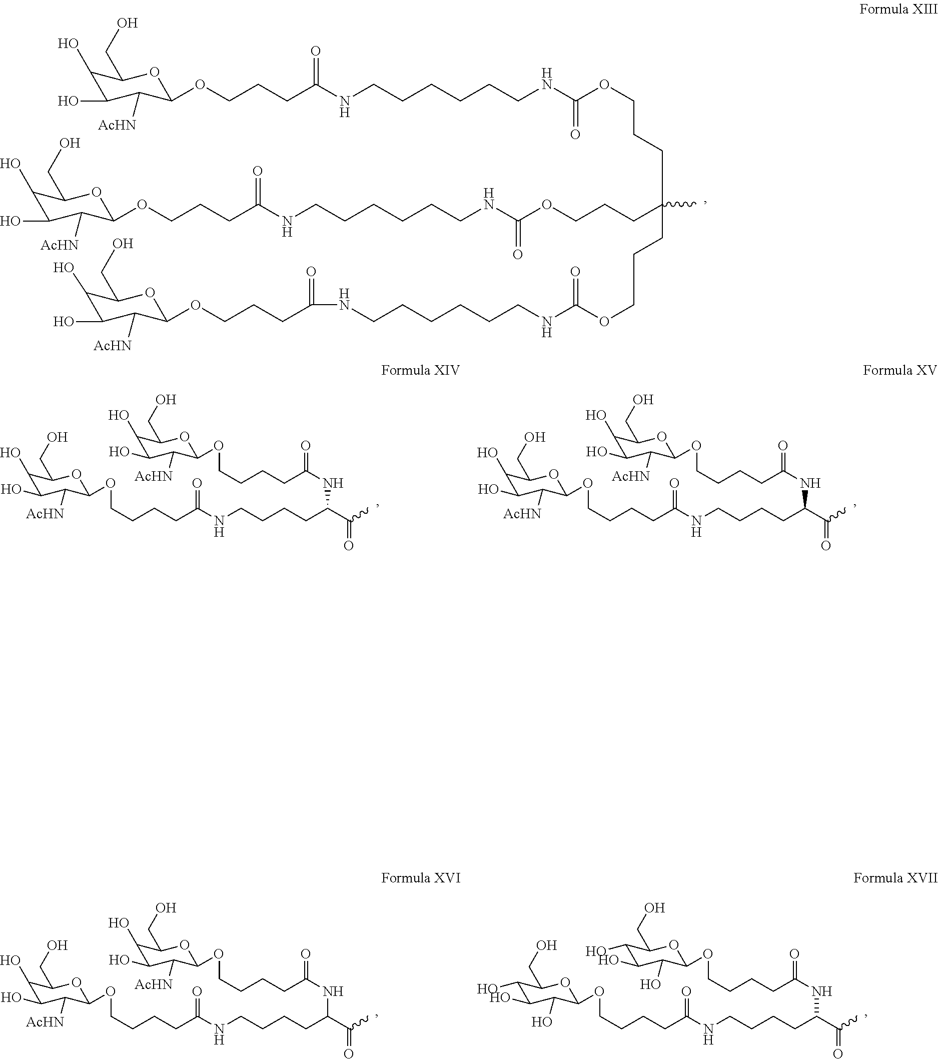

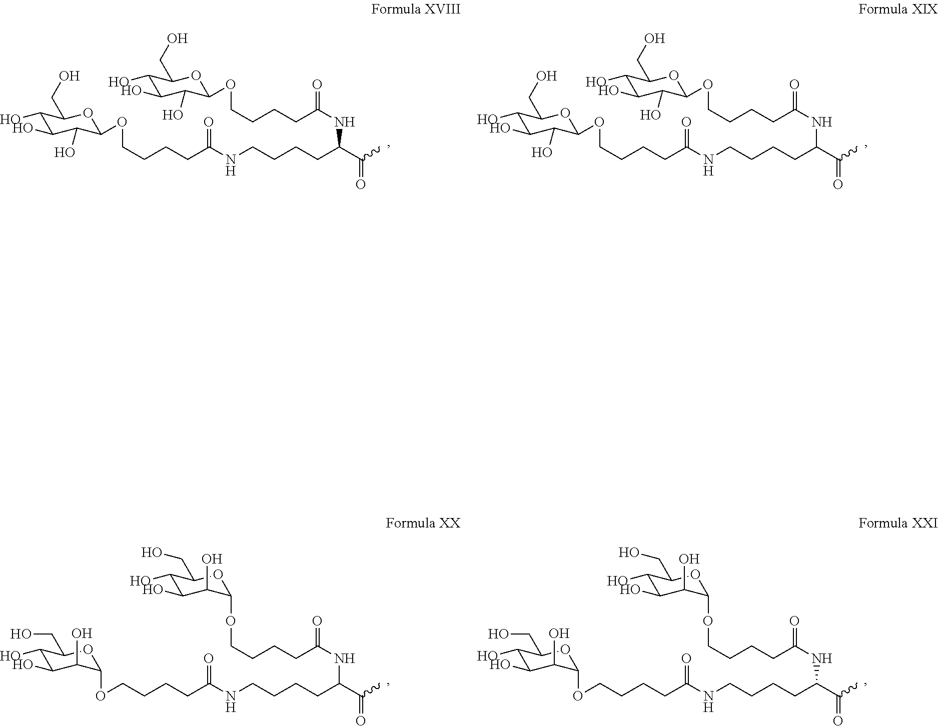

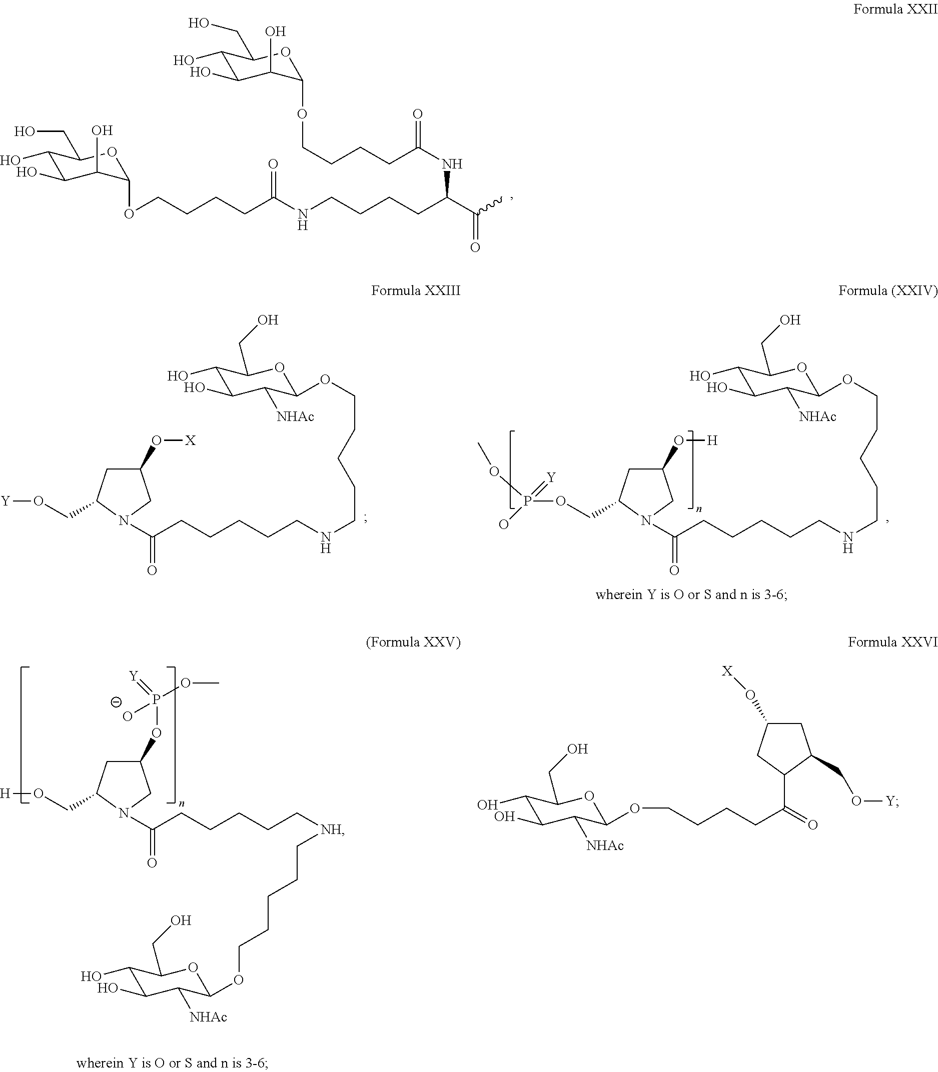

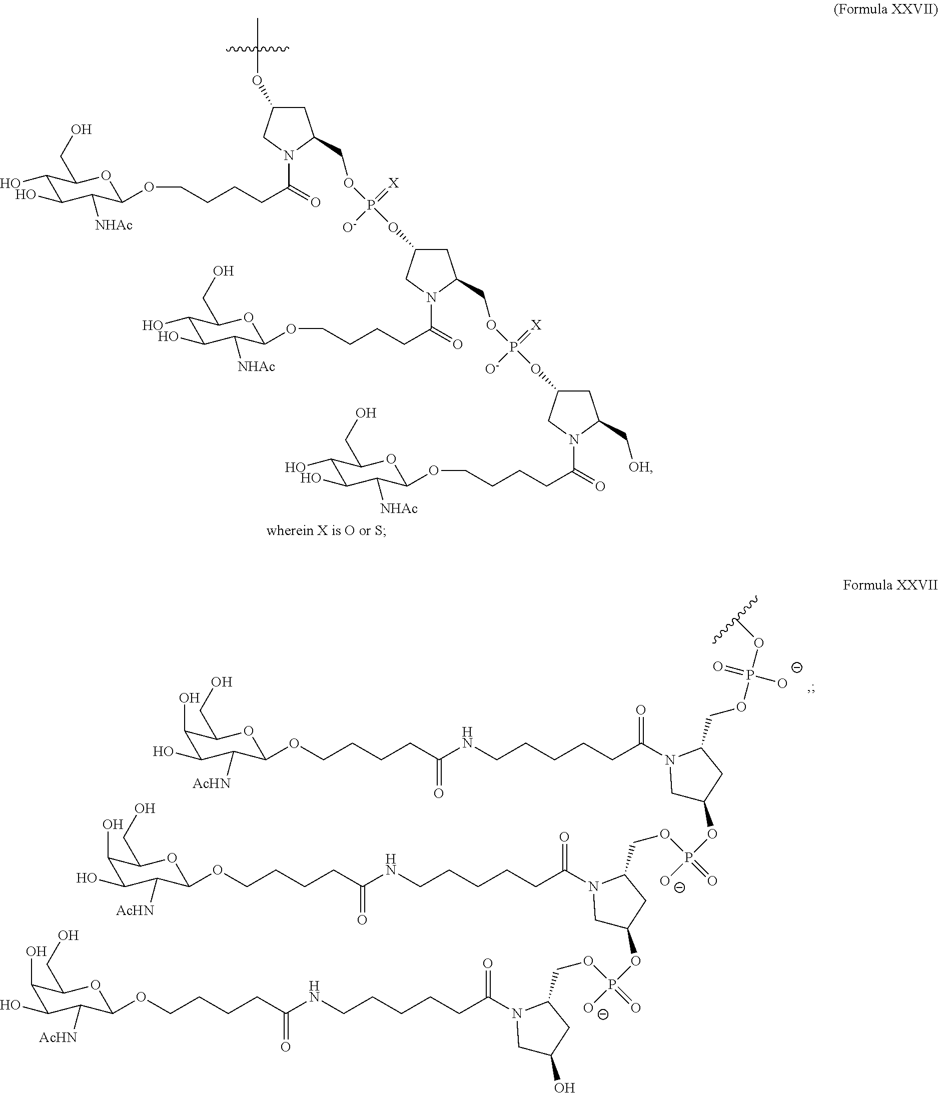

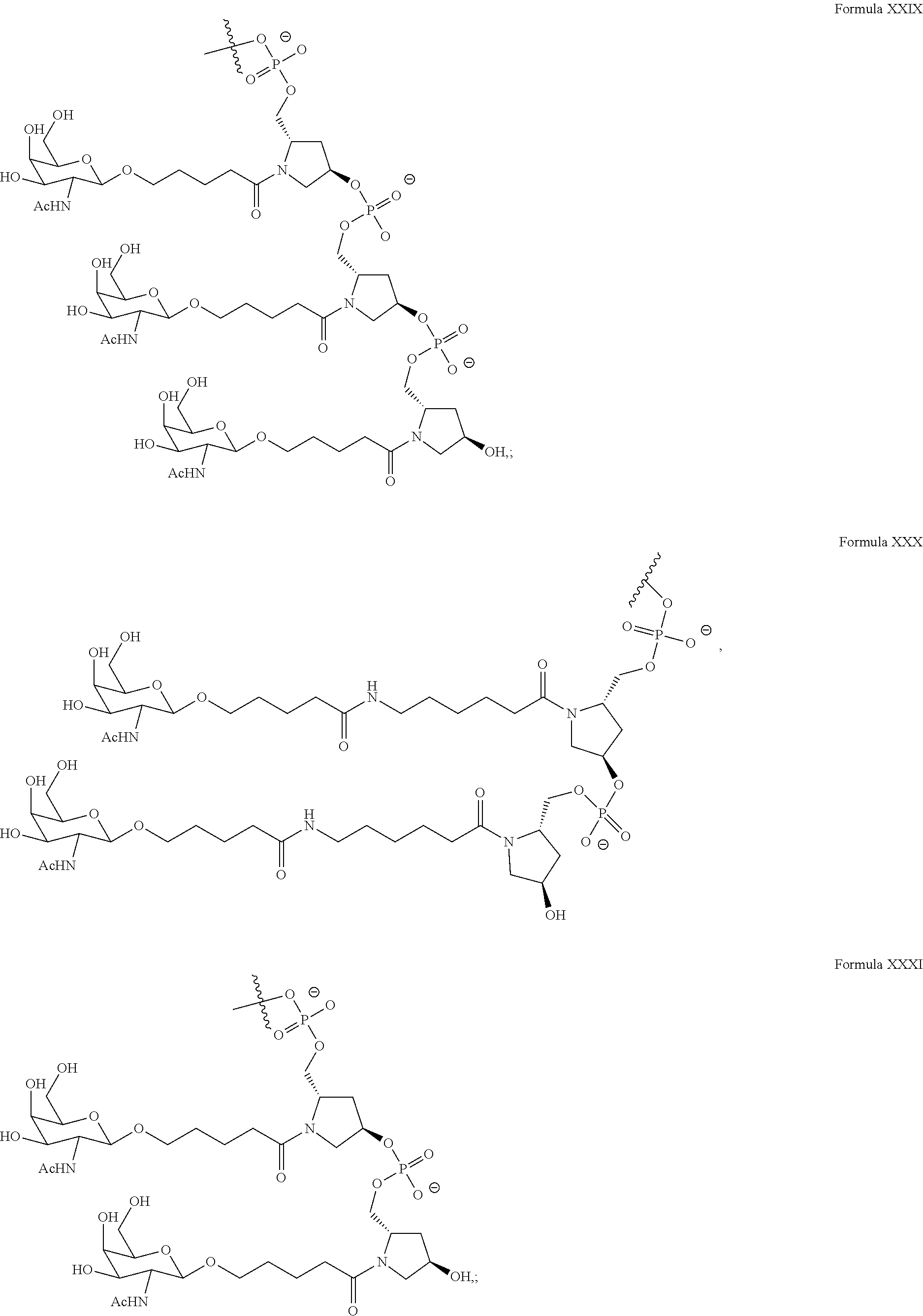

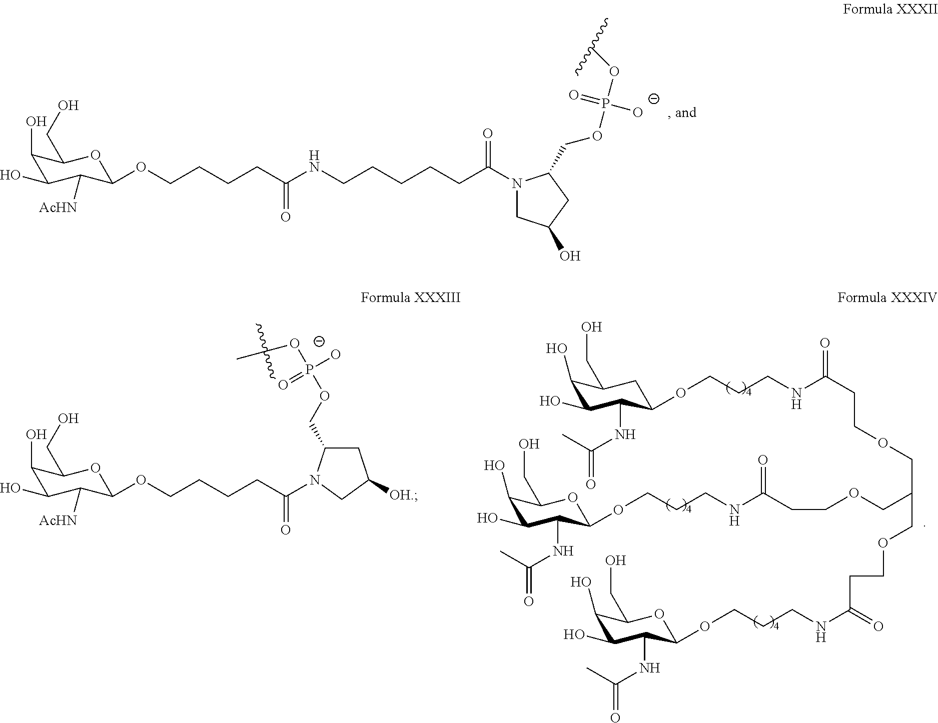

In certain embodiments, a carbohydrate conjugate for use in the compositions and methods of the invention is a monosaccharide. In certain embodiments, the monosaccharide is an N-acetylgalactosamine, such as

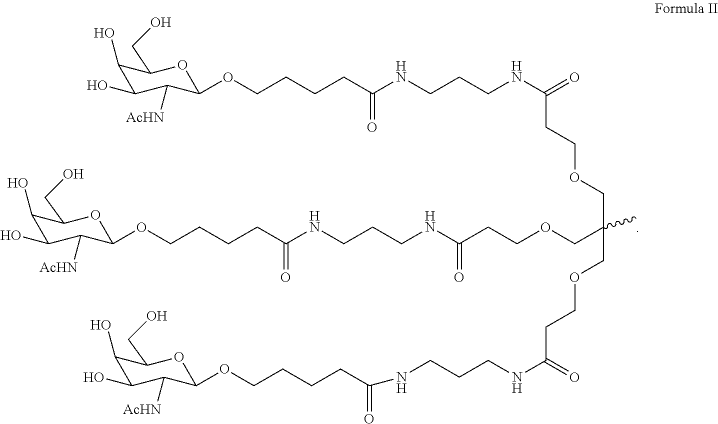 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

Another representative carbohydrate conjugate for use in the embodiments described herein includes, but is not limited to,

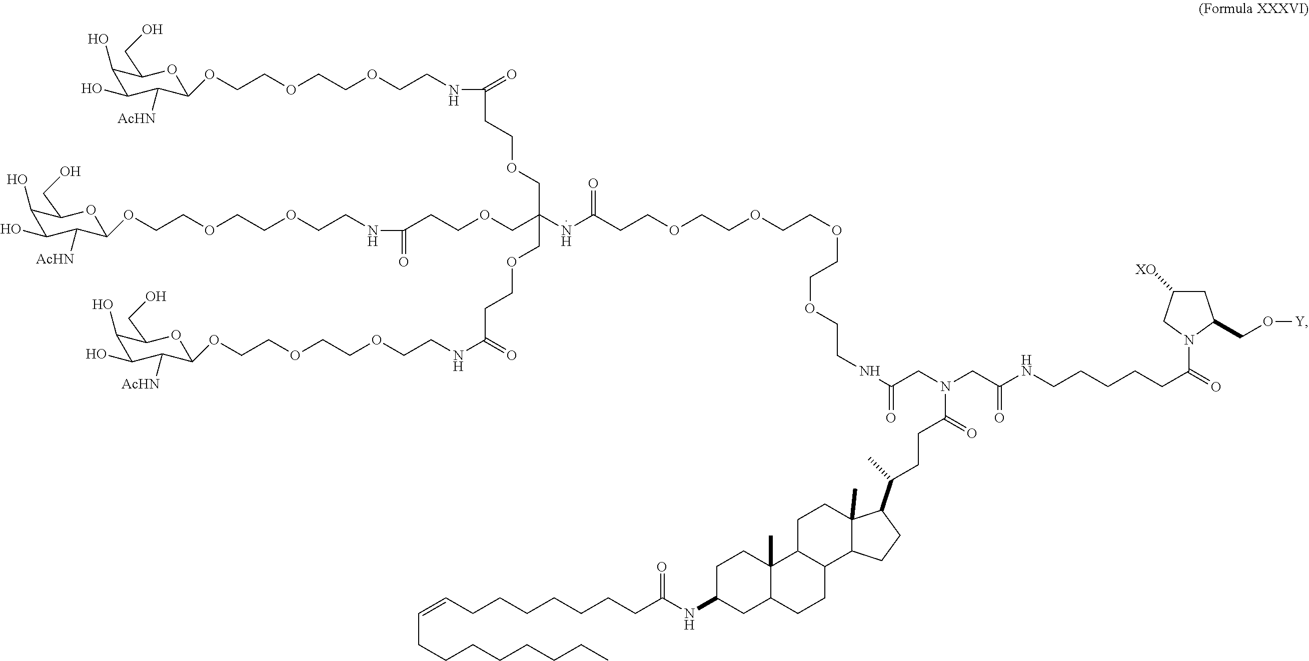

when one of X or Y is an oligonucleotide, the other is a hydrogen.

In some embodiments, a suitable ligand is a ligand disclosed in WO 2019/055633, the entire contents of which are incorporated herein by reference. In one embodiment the ligand comprises the structure below:

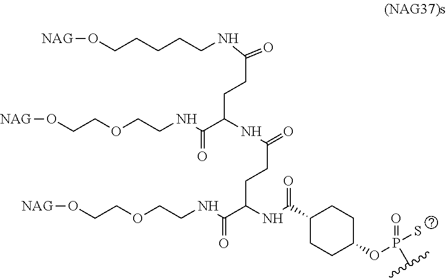

In certain embodiments, the nucleic acid inhibitors of the disclosure may include GalNAc ligands, even if such GalNAc ligands are currently projected to be of limited value for the intrathecal/CNS delivery route(s) of the instant disclosure.

In some embodiments, the carbohydrate conjugate further comprises one or more additional ligands as described above, such as, but not limited to, a PK modulator or a cell permeation peptide.

Additional carbohydrate conjugates and linkers suitable for use in the present invention include those described in WO 2014/179620 and WO 2014/179627, the entire contents of each of which are incorporated herein by reference.

In embodiments in which a first dsRNA agent targeting LDHA and a second dsRNA agent targeting HAO1 are covalently attached (i.e., a dual targeting RNAi agent), one or both of the dsRNA agents may independently comprise a GalNAc or GalNAc derivative ligand.

iv. Linkers

In some embodiments, the conjugate or ligand described herein can be attached to a nucleic acid inhibitor with various linkers that can be cleavable or non cleavable.

The term “linker” or “linking group” means an organic moiety that connects two parts of a compound, e.g., covalently attaches two parts of a compound. Linkers typically comprise a direct bond or an atom such as oxygen or sulfur, a unit such as NR8, C(O), C(O)NH, SO, SO₂, SO₂NH or a chain of atoms, such as, but not limited to, substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, alkynylhereroaryl, which one or more methylenes can be interrupted or terminated by O, S, S(O), SO₂, N(R8), C(O), substituted or unsubstituted aryl, substituted or unsubstituted heteroaryl, substituted or unsubstituted heterocyclic; where R8 is hydrogen, acyl, aliphatic or substituted aliphatic. In one embodiment, the linker is between about 1-24 atoms, 2-24, 3-24, 4-24, 5-24, 6-24, 6-18, 7-18, 8-18 atoms, 7-17, 8-17, 6-16, 7-17, or 8-16 atoms.

A cleavable linking group is one which is sufficiently stable outside the cell, but which upon entry into a target cell is cleaved to release the two parts the linker is holding together. In one embodiment, the cleavable linking group is cleaved at least about 10 times, 20, times, 30 times, 40 times, 50 times, 60 times, 70 times, 80 times, 90 times or more, or at least about 100 times faster in a target cell or under a first reference condition (which can, e.g., be selected to mimic or represent intracellular conditions) than in the blood of a subject, or under a second reference condition (which can, e.g., be selected to mimic or represent conditions found in the blood or serum).

Cleavable linking groups are susceptible to cleavage agents, e.g., pH, redox potential or the presence of degradative molecules. Generally, cleavage agents are more prevalent or found at higher levels or activities inside cells than in serum or blood. Examples of such degradative agents include: redox agents which are selected for particular substrates or which have no substrate specificity, including, e.g., oxidative or reductive enzymes or reductive agents such as mercaptans, present in cells, that can degrade a redox cleavable linking group by reduction; esterases; endosomes or agents that can create an acidic environment, e.g., those that result in a pH of five or lower; enzymes that can hydrolyze or degrade an acid cleavable linking group by acting as a general acid, peptidases (which can be substrate specific), and phosphatases.

A cleavable linkage group, such as a disulfide bond can be susceptible to pH. The pH of human serum is 7.4, while the average intracellular pH is slightly lower, ranging from about 7.1-7.3. Endosomes have a more acidic pH, in the range of 5.5-6.0, and lysosomes have an even more acidic pH at around 5.0. Some linkers will have a cleavable linking group that is cleaved at a certain pH, thereby releasing a cationic lipid from the ligand inside the cell, or into the desired compartment of the cell.

A linker can include a cleavable linking group that is cleavable by a particular enzyme. The type of cleavable linking group incorporated into a linker can depend on the cell to be targeted. For example, a liver-targeting ligand can be linked to a cationic lipid through a linker that includes an ester group. Liver cells are rich in esterases, and therefore the linker will be cleaved more efficiently in liver cells than in cell types that are not esterase-rich. Other cell-types rich in esterases include cells of the lung, renal cortex, and testis.

Linkers that contain peptide bonds can be used when targeting cell types rich in peptidases, such as liver cells and synoviocytes.

In general, the suitability of a candidate cleavable linking group can be evaluated by testing the ability of a degradative agent (or condition) to cleave the candidate linking group. It will also be desirable to also test the candidate cleavable linking group for the ability to resist cleavage in the blood or when in contact with other non-target tissue. Thus, one can determine the relative susceptibility to cleavage between a first and a second condition, where the first is selected to be indicative of cleavage in a target cell and the second is selected to be indicative of cleavage in other tissues or biological fluids, e.g., blood or serum. The evaluations can be carried out in cell free systems, in cells, in cell culture, in organ or tissue culture, or in whole animals. It can be useful to make initial evaluations in cell-free or culture conditions and to confirm by further evaluations in whole animals. In certain embodiments, useful candidate compounds are cleaved at least about 2, 4, 10, 20, 30, 40, 50, 60, 70, 80, 90, or about 100 times faster in the cell (or under in vitro conditions selected to mimic intracellular conditions) as compared to blood or serum (or under in vitro conditions selected to mimic extracellular conditions).

a. Redox Cleavable Linking Groups

In one embodiment, a cleavable linking group is a redox cleavable linking group that is cleaved upon reduction or oxidation. An example of reductively cleavable linking group is a disulphide linking group (—S—S—). To determine if a candidate cleavable linking group is a suitable “reductively cleavable linking group,” or for example is suitable for use with a particular nucleic acid inhibitor and particular targeting agent one can look to methods described herein. For example, a candidate can be evaluated by incubation with dithiothreitol (DTT), or other reducing agent using reagents know in the art, which mimic the rate of cleavage which would be observed in a cell, e.g., a target cell. The candidates can also be evaluated under conditions which are selected to mimic blood or serum conditions. In one, candidate compounds are cleaved by at most about 10% in the blood. In other embodiments, useful candidate compounds are degraded at least about 2, 4, 10, 20, 30, 40, 50, 60, 70, 80, 90, or about 100 times faster in the cell (or under in vitro conditions selected to mimic intracellular conditions) as compared to blood (or under in vitro conditions selected to mimic extracellular conditions). The rate of cleavage of candidate compounds can be determined using standard enzyme kinetics assays under conditions chosen to mimic intracellular media and compared to conditions chosen to mimic extracellular media.

b. Phosphate-Based Cleavable Linking Groups

In other embodiments, a cleavable linker comprises a phosphate-based cleavable linking group. A phosphate-based cleavable linking group is cleaved by agents that degrade or hydrolyze the phosphate group. An example of an agent that cleaves phosphate groups in cells are enzymes such as phosphatases in cells. Examples of phosphate-based linking groups are —O—P(O)(ORk)-O—, —O—P(S)(ORk)-O—, —O—P(S)(SRk)-O—, —S—P(O)(ORk)-O—, —O—P(O)(ORk)-S—, —S—P(O)(ORk)-S—, —O—P(S)(ORk)-S—, —S—P(S)(ORk)-O—, —O—P(O)(Rk)-O—, —O—P(S)(Rk)-O—, —S—P(O)(Rk)-O—, —S—P(S)(Rk)-O—, —S—P(O)(Rk)-S—, —O—P(S)(Rk)-S—, wherein Rk at each occurrence can be, independently, C1-C20 alkyl, C1-C20 haloalkyl, C6-C10 aryl, or C7-C12 aralkyl. Exemplary embodiments include —O—P(O)(OH)—O—, —O—P(S)(OH)—O—, —O—P(S)(SH)—O—, —S—P(O)(OH)—O—, —O—P(O)(OH)—S—, —S—P(O)(OH)—S—, —O—P(S)(OH)—S—, —S—P(S)(OH)—O—, —O—P(O)(H)—O—, —O—P(S)(H)—O—, —S—P(O)(H)—O, —S—P(S)(H)—O—, —S—P(O)(H)—S—, and —O—P(S)(H)—S—. In certain embodiments a phosphate-based linking group is —O—P(O)(OH)—O—. These candidates can be evaluated using methods analogous to those described above.

c. Acid Cleavable Linking Groups

In another embodiment, a cleavable linker comprises an acid cleavable linking group. An acid cleavable linking group is a linking group that is cleaved under acidic conditions. In one embodiments acid cleavable linking groups are cleaved in an acidic environment with a pH of about 6.5 or lower (e.g., about 6.0, 5.75, 5.5, 5.25, 5.0, or lower), or by agents such as enzymes that can act as a general acid. In a cell, specific low pH organelles, such as endosomes and lysosomes can provide a cleaving environment for acid cleavable linking groups. Examples of acid cleavable linking groups include but are not limited to hydrazones, esters, and esters of amino acids. Acid cleavable groups can have the general formula —C═NN—, C(O)O, or —OC(O). An exemplary embodiment is when the carbon attached to the oxygen of the ester (the alkoxy group) is an aryl group, substituted alkyl group, or tertiary alkyl group such as dimethyl pentyl or t-butyl. These candidates can be evaluated using methods analogous to those described above.

d. Ester-Based Linking Groups

In another embodiment, a cleavable linker comprises an ester-based cleavable linking group. An ester-based cleavable linking group is cleaved by enzymes such as esterases and amidases in cells. Examples of ester-based cleavable linking groups include but are not limited to esters of alkylene, alkenylene and alkynylene groups. Ester cleavable linking groups have the general formula —C(O)O—, or —OC(O)—. These candidates can be evaluated using methods analogous to those described above.

e. Peptide-Based Cleaving Groups

In yet another embodiment, a cleavable linker comprises a peptide-based cleavable linking group. A peptide-based cleavable linking group is cleaved by enzymes such as peptidases and proteases in cells. Peptide-based cleavable linking groups are peptide bonds formed between amino acids to yield oligopeptides (e.g., dipeptides, tripeptides etc.) and polypeptides. Peptide-based cleavable groups do not include the amide group (—C(O)NH—). The amide group can be formed between any alkylene, alkenylene or alkynelene. A peptide bond is a special type of amide bond formed between amino acids to yield peptides and proteins. The peptide based cleavage group is generally limited to the peptide bond (i.e., the amide bond) formed between amino acids yielding peptides and proteins and does not include the entire amide functional group. Peptide-based cleavable linking groups have the general formula —NHCHRAC(O)NHCHRBC(O)—, where RA and RB are the R groups of the two adjacent amino acids. These candidates can be evaluated using methods analogous to those described above.

In one embodiment, a nucleic acid inhibitor of the invention is conjugated to a carbohydrate through a linker. Non-limiting examples of carbohydrate conjugates with linkers of the compositions and methods of the invention include, but are not limited to,

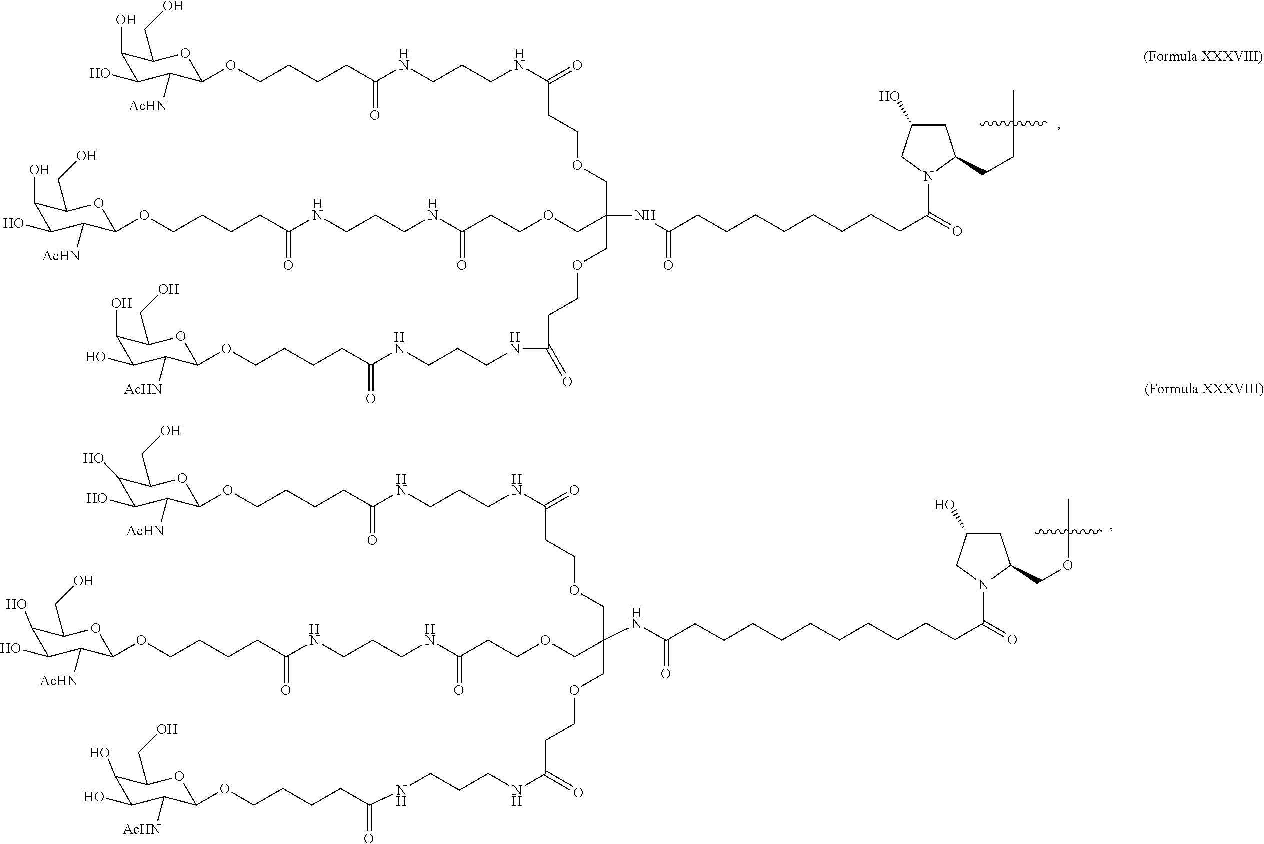

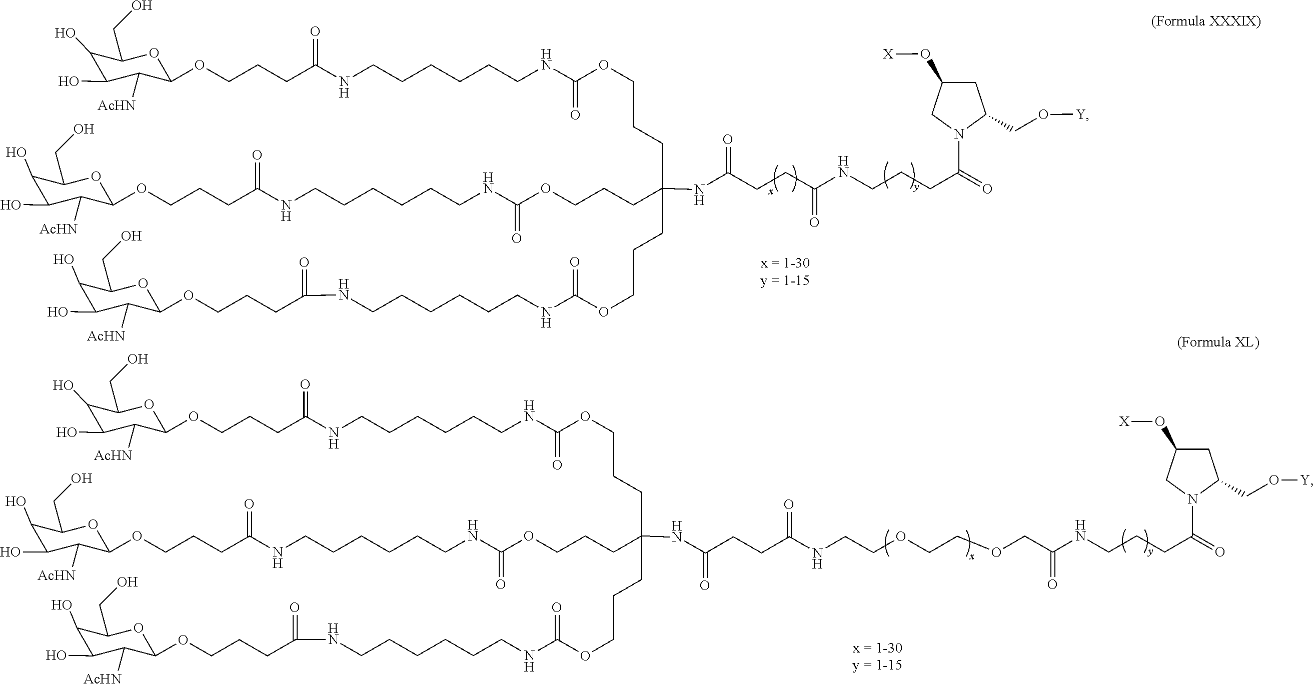

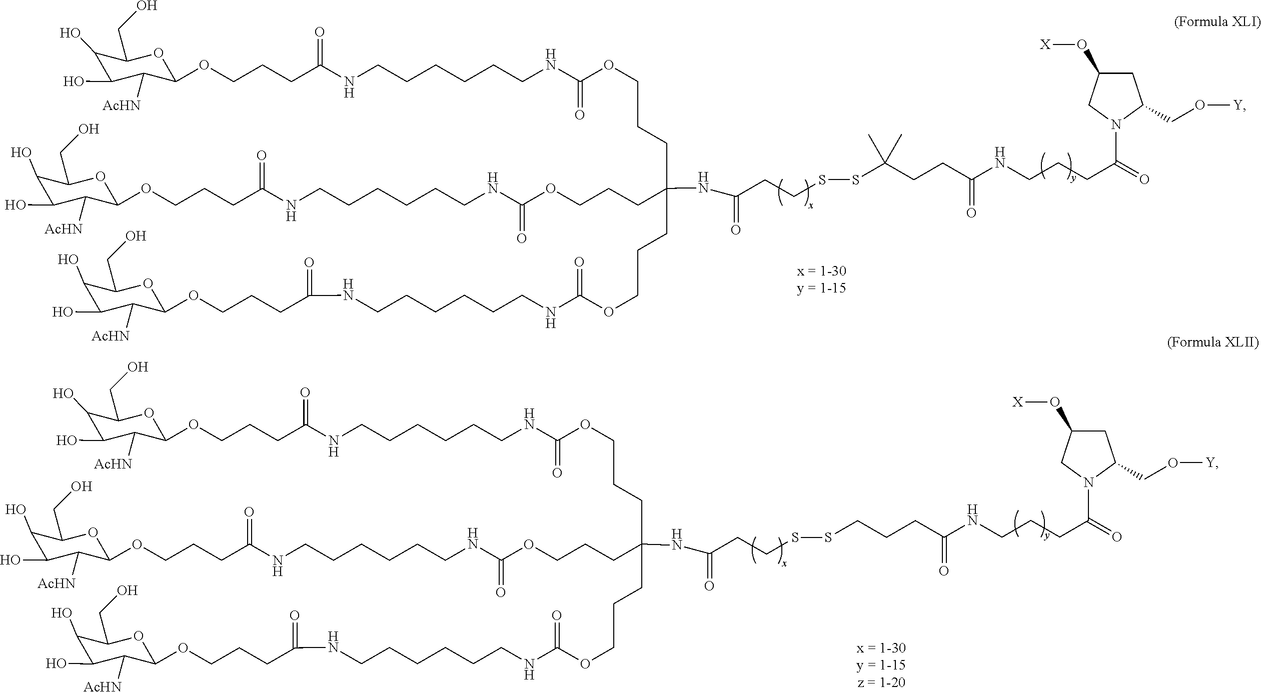

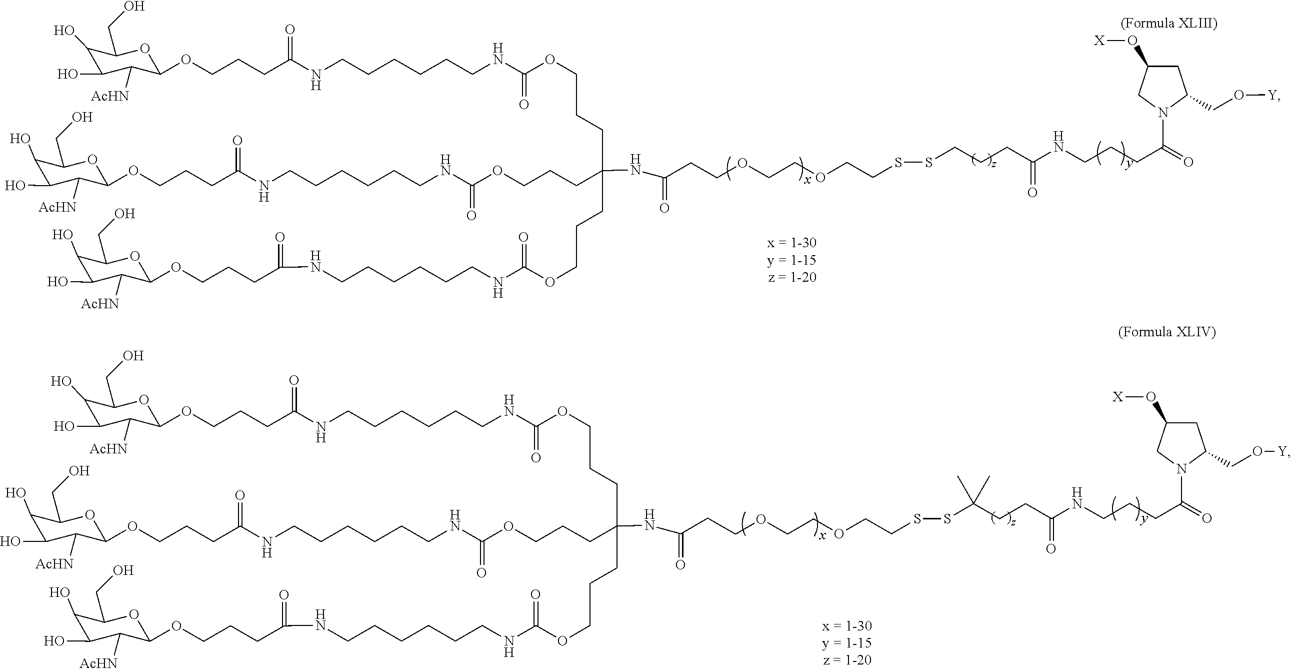

when one of X or Y is an oligonucleotide, the other is a hydrogen.

In certain embodiments of the compositions and methods of the invention, a ligand is one or more GalNAc (N-acetylgalactosamine) derivatives attached through a bivalent or trivalent branched linker.

In embodiments in which a first dsRNA agent targeting LDHA and a second dsRNA agent targeting HAO1 are covalently attached (i.e., a dual targeting RNAi agent), one or both of the dsRNA agents may independently a ligand comprising one or more GalNAc (N-acetylgalactosamine) derivatives attached through a bivalent or trivalent branched linker.

In one embodiment, a nucleic acid inhibitor of the invention is conjugated to a bivalent or trivalent branched linker selected from the group of structures shown in any of formula (XLV)-(XLVIII):

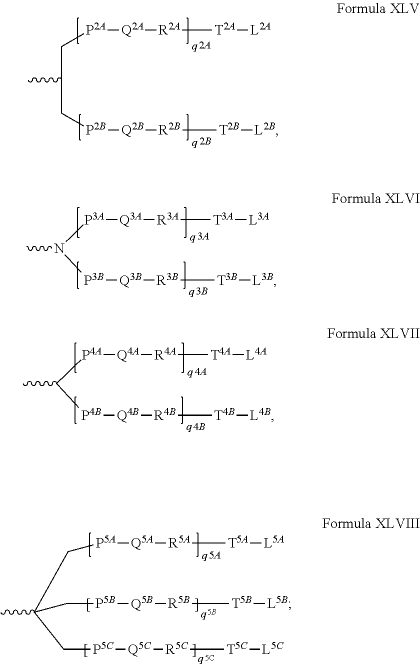

wherein:
q2A, q2B, q3A, q3B, q4A, q4B, q5A, q5B and q5C represent independently for each occurrence 0-20 and wherein the repeating unit can be the same or different;
P^{2A}, P^{2B}, P^{3A}, P^{3B}, P^{4A}, P^{4B}, P^{5A}, P^{5B}, P^{5C}, T^{2A}, T^{2B}, T^{3A}, T^{3B}, T^{4A}, T^{4B}, T^{4A}, T^{5B}, T^{5C} are each independently for each occurrence absent, CO, NH, O, S, OC(O), NHC(O), CH₂, CH₂NH or CH₂O;
Q^{2A}, Q^{2B}, Q^{3A}, Q^{3B}, Q^{4A}, Q^{4B}, Q^{5A}, Q^{5B}, Q^{5C} are independently for each occurrence absent, alkylene, substituted alkylene wherein one or more methylenes can be interrupted or terminated by one or more of O, S, S(O), SO₂, N(R^{N}), C(R′)═C(R″), C≡C or C(O);
R^{2A}, R^{2B}, R^{3A}, R^{3B}, R^{4A}, R^{4B}, R^{5A}, R^{5B}, R^{5C} are each independently for each occurrence absent, NH, O, S, CH₂, C(O)O, C(O)NH, NHCH(R^{a})C(O), —C(O)—CH(R^{a})—NH—, CO, CH═N—O,

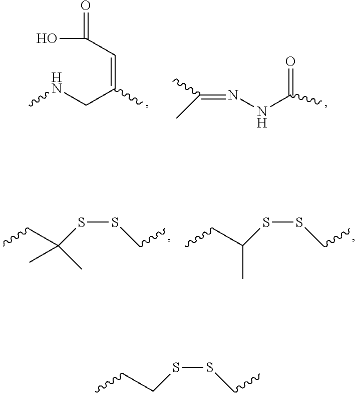

or heterocyclyl;

L^{2A}, L^{2B}, L^{3A}, L^{3B}, L^{4A}, L^{4B}, L^{5A}, L^{5B} and L^{5C} represent the ligand; i.e. each independently for each occurrence a monosaccharide (such as GalNAc), disaccharide, trisaccharide, tetrasaccharide, oligosaccharide, or polysaccharide; and R^{a} is H or amino acid side chain. Trivalent conjugating GalNAc derivatives are particularly useful for use with RNAi agents for inhibiting the expression of a target gene, such as those of formula (XLIX):

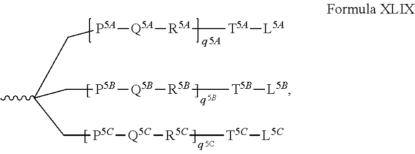

wherein L^{5A}, L^{5B} and L^{5C} represent a monosaccharide, such as GalNAc derivative.

Examples of suitable bivalent and trivalent branched linker groups conjugating GalNAc derivatives include, but are not limited to, the structures recited above as formulas II, VII, XI, X, and XIII.

Representative U.S. patents that teach the preparation of conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941; 6,294,664; 6,320,017; 6,576,752; 6,783,931; 6,900,297; 7,037,646; 8,106,022, the entire contents of each of which are hereby incorporated herein by reference.

It is not necessary for all positions in a given compound to be uniformly modified, and in fact more than one of the aforementioned modifications can be incorporated in a single compound or even at a single nucleoside within a nucleic acid inhibitor. The present invention also includes nucleic acid inhibitors that are chimeric compounds.

“Chimeric” iRNA compounds or “chimeras,” in the context of this invention, are nucleic acid inhibitors which contain two or more chemically distinct regions, each made up of at least one monomer unit, i.e., a nucleotide in the case of a dsRNA compound. These nucleic acid inhibitors typically contain at least one region wherein the RNA is modified so as to confer upon the nucleic acid inhibitor increased resistance to nuclease degradation, increased cellular uptake, and/or increased binding affinity for the target nucleic acid. An additional region of the nucleic acid inhibitor can serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of iRNA inhibition of gene expression. Consequently, comparable results can often be obtained with shorter iRNAs when chimeric dsRNAs are used, compared to phosphorothioate deoxy dsRNAs hybridizing to the same target region. Cleavage of the RNA target can be routinely detected by gel electrophoresis and, if necessary, associated nucleic acid hybridization techniques known in the art.

In certain instances, the RNA of a nucleic acid inhibitor can be modified by a non-ligand group. A number of non-ligand molecules have been conjugated to iRNAs in order to enhance the activity, cellular distribution or cellular uptake of the iRNA, and procedures for performing such conjugations are available in the scientific literature. Such non-ligand moieties have included lipid moieties, such as cholesterol (Kubo, T. et al., Biochem. Biophys. Res. Comm., 2007, 365(1):54-61; Letsinger et al., Proc. Natl. Acad. Sci. USA, 1989, 86:6553), cholic acid (Manoharan et al., Bioorg. Med. Chem. Lett., 1994, 4:1053), a thioether, e.g., hexyl-S-tritylthiol (Manoharan et al., Ann. N.Y. Acad. Sci., 1992, 660:306; Manoharan et al., Bioorg. Med. Chem. Let., 1993, 3:2765), a thiocholesterol (Oberhauser et al., Nucl. Acids Res., 1992, 20:533), an aliphatic chain, e.g., dodecandiol or undecyl residues (Saison-Behmoaras et al., EMBO J., 1991, 10:111; Kabanov et al., FEBS Lett., 1990, 259:327; Svinarchuk et al., Biochimie, 1993, 75:49), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36:3651; Shea et al., Nucl. Acids Res., 1990, 18:3777), a polyamine or a polyethylene glycol chain (Manoharan et al., Nucleosides & Nucleotides, 1995, 14:969), or adamantane acetic acid (Manoharan et al., Tetrahedron Lett., 1995, 36:3651), a palmityl moiety (Mishra et al., Biochim. Biophys. Acta, 1995, 1264:229), or an octadecylamine or hexylamino-carbonyl-oxycholesterol moiety (Crooke et al., J. Pharmacol. Exp. Ther., 1996, 277:923). Representative United States patents that teach the preparation of such RNA conjugates have been listed above. Typical conjugation protocols involve the synthesis of an RNAs bearing an aminolinker at one or more positions of the sequence. The amino group is then reacted with the molecule being conjugated using appropriate coupling or activating reagents. The conjugation reaction can be performed either with the RNA still bound to the solid support or following cleavage of the RNA, in solution phase. Purification of the RNA conjugate by HPLC typically affords the pure conjugate.

### III. Delivery of a Nucleic Acid Inhibitor of the Invention

The delivery of a nucleic acid inhibitor of the invention to a cell e.g., a cell within a subject, such as a human subject (e.g., a subject in need thereof, such as a subject having or at risk of developing a non-primary hyperoxaluria disease or disorder) can be achieved in a number of different ways. For example, delivery may be performed by contacting a cell with a nucleic acid inhibitor of the invention either in vitro or in vivo. In vivo delivery may also be performed directly by administering a composition comprising a nucleic acid inhibitor, e.g., a dsRNA, to a subject.

Alternatively, in vivo delivery may be performed indirectly by administering one or more vectors that encode and direct the expression of the nucleic acid inhibitor. These alternatives are discussed further below.

In the methods of the invention which include a first dsRNA agent targeting LDHA and a second dsRNA agent targeting HAO1 which are covalently attached (i.e., a dual targeting RNAi agent), the delivery of the first agent may be the same or different than the delivery of the second agent.

In general, any method of delivering a nucleic acid molecule (in vitro or in vivo) can be adapted for use with a nucleic acid inhibitor of the invention (see e.g., Akhtar S. and Julian R L., (1992) Trends Cell. Biol. 2(5):139-144 and WO94/02595, which are incorporated herein by reference in their entireties). For in vivo delivery, factors to consider in order to deliver a nucleic acid inhibitor include, for example, biological stability of the delivered molecule, prevention of non-specific effects, and accumulation of the delivered molecule in the target tissue. The non-specific effects of a nucleic acid inhibitor can be minimized by local administration, for example, by direct injection or implantation into a tissue or topically administering the preparation. Local administration to a treatment site maximizes local concentration of the agent, limits the exposure of the agent to systemic tissues that can otherwise be harmed by the agent or that can degrade the agent, and permits a lower total dose of the nucleic acid inhibitor to be administered. Several studies have shown successful knockdown of gene products when a nucleic acid inhibitor is administered locally. For example, intraocular delivery of a VEGF dsRNA by intravitreal injection in cynomolgus monkeys (Tolentino, M J. et al., (2004) Retina 24:132-138) and subretinal injections in mice (Reich, S J. et al. (2003) Mol. Vis. 9:210-216) were both shown to prevent neovascularization in an experimental model of age-related macular degeneration. In addition, direct intratumoral injection of a dsRNA in mice reduces tumor volume (Pille, J. et al. (2005) Mol. Ther. 11:267-274) and can prolong survival of tumor-bearing mice (Kim, W J. et al., (2006) Mol. Ther. 14:343-350; Li, S. et al., (2007) Mol. Ther. 15:515-523). RNA interference has also shown success with local delivery to the CNS by direct injection (Dorn, G. et al., (2004) Nucleic Acids 32:e49; Tan, P H. et al. (2005) Gene Ther. 12:59-66; Makimura, H. et a.l (2002) BMC Neurosci. 3:18; Shishkina, G T., et al. (2004) Neuroscience 129:521-528; Thakker, E R., et al. (2004) Proc. Natl. Acad. Sci. U.S.A. 101:17270-17275; Akaneya, Y., et al. (2005) J. Neurophysiol. 93:594-602) and to the lungs by intranasal administration (Howard, K A. et al., (2006) Mol. Ther. 14:476-484; Zhang, X. et al., (2004) J. Biol. Chem. 279:10677-10684; Bitko, V. et al., (2005) Nat. Med. 11:50-55). For administering a nucleic acid inhibitor systemically for the treatment of a disease, the nucleic acid inhibitor can be modified or alternatively delivered using a drug delivery system; both methods act to prevent the rapid degradation of the nucleic acid inhibitor by endo- and exo-nucleases in vivo. Modification of the nucleic acid inhibitor or the pharmaceutical carrier can also permit targeting of the nucleic acid inhibitor to the target tissue and avoid undesirable off-target effects. Nucleic acid inhibitors can be modified by chemical conjugation to lipophilic groups such as cholesterol to enhance cellular uptake and prevent degradation. For example, a nucleic acid inhibitor directed against ApoB conjugated to a lipophilic cholesterol moiety was injected systemically into mice and resulted in knockdown of apoB mRNA in both the liver and jejunum (Soutschek, J. et al., (2004) Nature 432:173-178). Conjugation of an nucleic acid inhibitor to an aptamer has been shown to inhibit tumor growth and mediate tumor regression in a mouse model of prostate cancer (McNamara, J O. et al., (2006) Nat. Biotechnol. 24:1005-1015). In an alternative embodiment, the nucleic acid inhibitor can be delivered using drug delivery systems such as a nanoparticle, a dendrimer, a polymer, liposomes, or a cationic delivery system. Positively charged cationic delivery systems facilitate binding of a nucleic acid inhibitor (negatively charged) and also enhance interactions at the negatively charged cell membrane to permit efficient uptake of a nucleic acid inhibitor by the cell. Cationic lipids, dendrimers, or polymers can either be bound to a nucleic acid inhibitor, or induced to form a vesicle or micelle (see e.g., Kim S H. et al., (2008) Journal of Controlled Release 129(2):107-116) that encases a nucleic acid inhibitor. The formation of vesicles or micelles further prevents degradation of the iRNA when administered systemically. Methods for making and administering cationic-nucleic acid inhibitor complexes are well within the abilities of one skilled in the art (see e.g., Sorensen, D R., et al. (2003) J. Mol. Biol 327:761-766; Verma, U N. et al., (2003) Clin. Cancer Res. 9:1291-1300; Arnold, A S et al., (2007) J. Hypertens. 25:197-205, which are incorporated herein by reference in their entirety). Some non-limiting examples of drug delivery systems useful for systemic delivery of nucleic acid inhibitors include DOTAP (Sorensen, D R., et al (2003), supra; Verma, U N. et al., (2003), supra), Oligofectamine, “solid nucleic acid lipid particles” (Zimmermann, T S. et al., (2006) Nature 441:111-114), cardiolipin (Chien, P Y. et al., (2005) Cancer Gene Ther. 12:321-328; Pal, A. et al., (2005) Int J. Oncol. 26:1087-1091), polyethyleneimine (Bonnet M E. et al., (2008) Pharm. Res. August 16 Epub ahead of print; Aigner, A. (2006) J. Biomed. Biotechnol. 71659), Arg-Gly-Asp (RGD) peptides (Liu, S. (2006) Mol. Pharm. 3:472-487), and polyamidoamines (Tomalia, D A. et al., (2007) Biochem. Soc. Trans. 35:61-67; Yoo, H. et al., (1999) Pharm. Res. 16:1799-1804). In some embodiments, a nucleic acid inhibitor forms a complex with cyclodextrin for systemic administration. Methods for administration and pharmaceutical compositions of nucleic acid inhibitors and cyclodextrins can be found in U.S. Pat. No. 7,427,605, which is herein incorporated by reference in its entirety.

A. Vector Encoded iRNAs of the Invention

Nucleic acid inhibitors targeting the LDHA gene, nucleic acid inhibitor targeting the HAO1 gene, nucleic acid inhibitor targeting the PRODH2 gene, and nucleic acid inhibitors targeting LDHA and HAO1 can be expressed from transcription units inserted into DNA or RNA vectors (see, e.g. Couture, A, et al., TIG. (1996), 12:5-10; Skillern, A., et al., International PCT Publication No. WO 00/22113, Conrad, International PCT Publication No. WO 00/22114, and Conrad, U.S. Pat. No. 6,054,299). Expression can be transient (on the order of hours to weeks) or sustained (weeks to months or longer), depending upon the specific construct used and the target tissue or cell type. These transgenes can be introduced as a linear construct, a circular plasmid, or a viral vector, which can be an integrating or non-integrating vector. The transgene can also be constructed to permit it to be inherited as an extrachromosomal plasmid (Gassmann, et al., (1995) Proc. Natl. Acad. Sci. USA 92:1292).

The individual strand or strands of a nucleic acid inhibitor can be transcribed from a promoter on an expression vector. Where two separate strands are to be expressed to generate, for example, a dsRNA, two separate expression vectors can be co-introduced (e.g., by transfection or infection) into a target cell. Alternatively each individual strand of a nucleic acid inhibitor can be transcribed by promoters both of which are located on the same expression plasmid. In one embodiment, a dsRNA is expressed as inverted repeat polynucleotides joined by a linker polynucleotide sequence such that the dsRNA has a stem and loop structure.

Nucleic acid inhibitor expression vectors are generally DNA plasmids or viral vectors. Expression vectors compatible with eukaryotic cells, such as those compatible with vertebrate cells, can be used to produce recombinant constructs for the expression of a nucleic acid inhibitor as described herein. Eukaryotic cell expression vectors are well known in the art and are available from a number of commercial sources. Typically, such vectors are provided containing convenient restriction sites for insertion of the desired nucleic acid segment. Delivery of nucleic acid inhibitor expressing vectors can be systemic, such as by intravenous or intramuscular administration, by administration to target cells ex-planted from the patient followed by reintroduction into the patient, or by any other means that allows for introduction into a desired target cell.

Viral vector systems which can be utilized with the methods and compositions described herein include, but are not limited to, (a) adenovirus vectors; (b) retrovirus vectors, including but not limited to lentiviral vectors, moloney murine leukemia virus, etc.; (c) adeno-associated virus vectors; (d) herpes simplex virus vectors; (e) SV 40 vectors; (f) polyoma virus vectors; (g) papilloma virus vectors; (h) picornavirus vectors; (i) pox virus vectors such as an orthopox, e.g., vaccinia virus vectors or avipox, e.g. canary pox or fowl pox; and (j) a helper-dependent or gutless adenovirus. Replication-defective viruses can also be advantageous. Different vectors will or will not become incorporated into the cells' genome. The constructs can include viral sequences for transfection, if desired. Alternatively, the construct can be incorporated into vectors capable of episomal replication, e.g. EPV and EBV vectors. Constructs for the recombinant expression of an iRNA will generally require regulatory elements, e.g., promoters, enhancers, etc., to ensure the expression of the iRNA in target cells. Other aspects to consider for vectors and constructs are known in the art.

### IV. Pharmaceutical Compositions of the Invention

The present invention also includes pharmaceutical compositions and formulations which include the nucleic acid inhibitors of the invention. Accordingly, in one embodiment, provided herein are pharmaceutical compositions comprising a nucleic acid inhibitor, such as a double stranded ribonucleic acid (dsRNA) agent or a single stranded antisense polynucleotide agent that inhibits expression of LDHA1 in a cell, such as a liver cell; and a pharmaceutically acceptable carrier.

In another embodiment, provided herein are pharmaceutical compositions comprising a nucleic acid inhibitor, such as a double stranded ribonucleic acid (dsRNA) agent or a single stranded antisense polynucleotide agent that inhibits expression of HAO1 in a cell, such as a liver cell; and a pharmaceutically acceptable carrier.

In another embodiment, provided herein are pharmaceutical compositions comprising a nucleic acid inhibitor, such as a double stranded ribonucleic acid (dsRNA) agent or a single stranded antisense polynucleotide agent that inhibits expression of PRODH2 in a cell, such as a liver cell; and a pharmaceutically acceptable carrier.

In one embodiment, provided herein are pharmaceutical compositions comprising a first nucleic acid inhibitor, such as a double stranded ribonucleic acid (dsRNA) agent or a single stranded antisense polynucleotide agent, that inhibits expression of lactic acid dehydrogenase A (LDHA) in a cell, such as a liver cell, and a second nucleic acid inhibitor, such as a double stranded ribonucleic acid (dsRNA) agent or a single stranded antisense polynucleotide agent, that inhibits expression of hydroxyacid oxidase 1 (glycolate oxidase) (HAO1) in a cell, such as a liver cell; and a pharmaceutically acceptable carrier.

In yet another embodiment, the present invention provides pharmaceutical compositions and formulations comprising a nucleic acid inhibitor, such as a dual targeting RNAi agent of the invention, and a pharmaceutically acceptable carrier.

The pharmaceutical compositions containing the iRNA of the invention are useful for treating a subject having or at risk of developing a non-primary hyperoxaluria disease or disorder.

Such pharmaceutical compositions are formulated based on the mode of delivery. One example is compositions that are formulated for systemic administration via parenteral delivery, e.g., by intravenous (IV) or for subcutaneous delivery. Another example is compositions that are formulated for direct delivery into the liver, e.g., by infusion into the liver, such as by continuous pump infusion.

The pharmaceutical compositions of the invention may be administered in dosages sufficient to inhibit expression of an LDHA gene, an HAO1 gene, a PRODH2 gene, or both an LDHA gene and an HAO1 gene. In general, a suitable dose of a nucleic acid inhibitor of the invention will be in the range of about 0.001 to about 200.0 milligrams per kilogram body weight of the recipient per day, generally in the range of about 1 to 50 mg per kilogram body weight per day. Typically, a suitable dose of a nucleic acid inhibitor of the invention will be in the range of about 0.1 mg/kg to about 5.0 mg/kg, such as, about 0.3 mg/kg and about 3.0 mg/kg.

In the methods of the invention which include a first nucleic acid inhibitor targeting LDHA and a second nucleic acid inhibitor targeting HAO1, the first inhibitor and the second inhibitor may be present in the same pharmaceutical formulation or separate pharmaceutical formulations.

A repeat-dose regimine may include administration of a therapeutic amount of nucleic acid inhibitor on a regular basis, such as every other day to once a year. In certain embodiments, the nucleic acid inhibitor is administered about once per month to about once per quarter (i.e., about once every three months).

After an initial treatment regimen, the treatments can be administered on a less frequent basis. The skilled artisan will appreciate that certain factors can influence the dosage and timing required to effectively treat a subject, including but not limited to the severity of the disease or disorder, previous treatments, the general health and/or age of the subject, and other diseases present. Moreover, treatment of a subject with a therapeutically effective amount of a composition can include a single treatment or a series of treatments. Estimates of effective dosages and in vivo half-lives for the individual nucleic acid inhibitors encompassed by the invention can be made using conventional methodologies or on the basis of in vivo testing using an appropriate animal model.

The pharmaceutical compositions of the present invention can be administered in a number of ways depending upon whether local or systemic treatment is desired and upon the area to be treated. Administration can be topical (e.g., by a transdermal patch), pulmonary, e.g., by inhalation or insufflation of powders or aerosols, including by nebulizer; intratracheal, intranasal, epidermal and transdermal, oral or parenteral. Parenteral administration includes intravenous, intraarterial, subcutaneous, intraperitoneal or intramuscular injection or infusion; subdermal, e.g., via an implanted device; or intracranial, e.g., by intraparenchymal, intrathecal or intraventricular, administration.

The nucleic acid inhibitor can be delivered in a manner to target a particular cell or tissue, such as the liver (e.g., the hepatocytes of the liver).

Pharmaceutical compositions and formulations for topical administration can include transdermal patches, ointments, lotions, creams, gels, drops, suppositories, sprays, liquids and powders. Conventional pharmaceutical carriers, aqueous, powder or oily bases, thickeners and the like can be necessary or desirable. Coated condoms, gloves and the like can also be useful. Suitable topical formulations include those in which the iRNAs featured in the invention are in admixture with a topical delivery agent such as lipids, liposomes, fatty acids, fatty acid esters, steroids, chelating agents and surfactants. Suitable lipids and liposomes include neutral (e.g., dioleoylphosphatidyl DOPE ethanolamine, dimyristoylphosphatidyl choline DMPC, distearolyphosphatidyl choline) negative (e.g., dimyristoylphosphatidyl glycerol DMPG) and cationic (e.g., dioleoyltetramethylaminopropyl DOTAP and dioleoylphosphatidyl ethanolamine DOTMA). Nucleic acid inhibitors featured in the invention can be encapsulated within liposomes or can form complexes thereto, in particular to cationic liposomes. Alternatively, nucleic acid inhibitors can be complexed to lipids, in particular to cationic lipids. Suitable fatty acids and esters include but are not limited to arachidonic acid, oleic acid, eicosanoic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a C₁₋₂₀ alkyl ester (e.g., isopropylmyristate IPM), monoglyceride, diglyceride or pharmaceutically acceptable salt thereof. Topical formulations are described in detail in U.S. Pat. No. 6,747,014, which is incorporated herein by reference.

Compositions and formulations for oral administration include powders or granules, microparticulates, nanoparticulates, suspensions or solutions in water or non-aqueous media, capsules, gel capsules, sachets, tablets or minitablets. Thickeners, flavoring agents, diluents, emulsifiers, dispersing aids or binders can be desirable. In some embodiments, oral formulations are those in which nucleic acid inhibitors featured in the invention are administered in conjunction with one or more penetration enhancer surfactants and chelators. Suitable surfactants include fatty acids and/or esters or salts thereof, bile acids and/or salts thereof. Suitable bile acids/salts include chenodeoxycholic acid (CDCA) and ursodeoxychenodeoxycholic acid (UDCA), cholic acid, dehydrocholic acid, deoxycholic acid, glucholic acid, glycholic acid, glycodeoxycholic acid, taurocholic acid, taurodeoxycholic acid, sodium tauro-24,25-dihydro-fusidate and sodium glycodihydrofusidate. Suitable fatty acids include arachidonic acid, undecanoic acid, oleic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a monoglyceride, a diglyceride or a pharmaceutically acceptable salt thereof (e.g., sodium). In some embodiments, combinations of penetration enhancers are used, for example, fatty acids/salts in combination with bile acids/salts. One exemplary combination is the sodium salt of lauric acid, capric acid and UDCA. Further penetration enhancers include polyoxyethylene-9-lauryl ether, polyoxyethylene-20-cetyl ether. DsRNAs featured in the invention can be delivered orally, in granular form including sprayed dried particles, or complexed to form micro or nanoparticles. DsRNA complexing agents include poly-amino acids; polyimines; polyacrylates; polyalkylacrylates, polyoxethanes, polyalkylcyanoacrylates; cationized gelatins, albumins, starches, acrylates, polyethyleneglycols (PEG) and starches; polyalkylcyanoacrylates; DEAE-derivatized polyimines, pollulans, celluloses and starches. Suitable complexing agents include chitosan, N-trimethylchitosan, poly-L-lysine, polyhistidine, polyornithine, polyspermines, protamine, polyvinylpyridine, polythiodiethylaminomethylethylene P(TDAE), polyaminostyrene (e.g., p-amino), poly(methylcyanoacrylate), poly(ethylcyanoacrylate), poly(butylcyanoacrylate), poly(isobutylcyanoacrylate), poly(isohexylcynaoacrylate), DEAE-methacrylate, DEAE-hexylacrylate, DEAE-acrylamide, DEAE-albumin and DEAE-dextran, polymethylacrylate, polyhexylacrylate, poly(D,L-lactic acid), poly(DL-lactic-co-glycolic acid (PLGA), alginate, and polyethyleneglycol (PEG). Oral formulations for dsRNAs and their preparation are described in detail in U.S. Pat. No. 6,887,906, US Publn. No. 20030027780, and U.S. Pat. No. 6,747,014, each of which is incorporated herein by reference.

Compositions and formulations for parenteral, intraparenchymal (into the brain), intrathecal, intraventricular or intrahepatic administration can include sterile aqueous solutions which can also contain buffers, diluents and other suitable additives such as, but not limited to, penetration enhancers, carrier compounds and other pharmaceutically acceptable carriers or excipients.

Pharmaceutical compositions of the present invention include, but are not limited to, solutions, emulsions, and liposome-containing formulations. These compositions can be generated from a variety of components that include, but are not limited to, preformed liquids, self-emulsifying solids and self-emulsifying semisolids.

The pharmaceutical formulations of the present invention, which can conveniently be presented in unit dosage form, can be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers or finely divided solid carriers or both, and then, if necessary, shaping the product.

The compositions of the present invention can be formulated into any of many possible dosage forms such as, but not limited to, tablets, capsules, gel capsules, liquid syrups, soft gels, suppositories, and enemas. The compositions of the present invention can also be formulated as suspensions in aqueous, non-aqueous or mixed media. Aqueous suspensions can further contain substances which increase the viscosity of the suspension including, for example, sodium carboxymethylcellulose, sorbitol and/or dextran. The suspension can also contain stabilizers.

### A. Additional Formulations

i. Emulsions

The compositions of the present invention can be prepared and formulated as emulsions. Emulsions are typically heterogeneous systems of one liquid dispersed in another in the form of droplets usually exceeding 0.1 μm in diameter (see e.g., Ansel's Pharmaceutical Dosage Forms and Drug Delivery Systems, Allen, L V., Popovich N G., and Ansel H C., 2004, Lippincott Williams & Wilkins (8th ed.), New York, NY; Idson, in Pharmaceutical Dosage Forms, Lieberman, Rieger and Banker (Eds.), 1988, Marcel Dekker, Inc., New York, N.Y., volume 1, p. 199; Rosoff, in Pharmaceutical Dosage Forms, Lieberman, Rieger and Banker (Eds.), 1988, Marcel Dekker, Inc., New York, N.Y., Volume 1, p. 245; Block in Pharmaceutical Dosage Forms, Lieberman, Rieger and Banker (Eds.), 1988, Marcel Dekker, Inc., New York, N.Y., volume 2, p. 335; Higuchi et al., in Remington's Pharmaceutical Sciences, Mack Publishing Co., Easton, Pa., 1985, p. 301). Emulsions are often biphasic systems comprising two immiscible liquid phases intimately mixed and dispersed with each other. In general, emulsions can be of either the water-in-oil (w/o) or the oil-in-water (o/w) variety. When an aqueous phase is finely divided into and dispersed as minute droplets into a bulk oily phase, the resulting composition is called a water-in-oil (w/o) emulsion. Alternatively, when an oily phase is finely divided into and dispersed as minute droplets into a bulk aqueous phase, the resulting composition is called an oil-in-water (o/w) emulsion. Emulsions can contain additional components in addition to the dispersed phases, and the active drug which can be present as a solution in either aqueous phase, oily phase or itself as a separate phase. Pharmaceutical excipients such as emulsifiers, stabilizers, dyes, and anti-oxidants can also be present in emulsions as needed.

Pharmaceutical emulsions can also be multiple emulsions that are comprised of more than two phases such as, for example, in the case of oil-in-water-in-oil (o/w/o) and water-in-oil-in-water (w/o/w) emulsions. Such complex formulations often provide certain advantages that simple binary emulsions do not. Multiple emulsions in which individual oil droplets of an o/w emulsion enclose small water droplets constitute a w/o/w emulsion. Likewise a system of oil droplets enclosed in globules of water stabilized in an oily continuous phase provides an o/w/o emulsion.

Emulsions are characterized by little or no thermodynamic stability. Often, the dispersed or discontinuous phase of the emulsion is well dispersed into the external or continuous phase and maintained in this form through the means of emulsifiers or the viscosity of the formulation. Either of the phases of the emulsion can be a semisolid or a solid, as is the case of emulsion-style ointment bases and creams. Other means of stabilizing emulsions entail the use of emulsifiers that can be incorporated into either phase of the emulsion. Emulsifiers can broadly be classified into four categories: synthetic surfactants, naturally occurring emulsifiers, absorption bases, and finely dispersed solids (see e.g., Ansel's Pharmaceutical Dosage Forms and Drug Delivery Systems, Allen, L V., Popovich N G., and Ansel H C., 2004, Lippincott Williams & Wilkins (8th ed.), New York, NY; Idson, in Pharmaceutical Dosage Forms, Lieberman, Rieger and Banker (Eds.), 1988, Marcel Dekker, Inc., New York, N.Y., volume 1, p. 199).

Synthetic surfactants, also known as surface active agents, have found wide applicability in the formulation of emulsions and have been reviewed in the literature (see e.g., Ansel's Pharmaceutical Dosage Forms and Drug Delivery Systems, Allen, L V., Popovich N G., and Ansel H C., 2004, Lippincott Williams & Wilkins (8th ed.), New York, NY; Rieger, in Pharmaceutical Dosage Forms, Lieberman, Rieger and Banker (Eds.), 1988, Marcel Dekker, Inc., New York, N.Y., volume 1, p. 285; Idson, in Pharmaceutical Dosage Forms, Lieberman, Rieger and Banker (Eds.), Marcel Dekker, Inc., New York, N.Y., 1988, volume 1, p. 199). Surfactants are typically amphiphilic and comprise a hydrophilic and a hydrophobic portion. The ratio of the hydrophilic to the hydrophobic nature of the surfactant has been termed the hydrophile/lipophile balance (HLB) and is a valuable tool in categorizing and selecting surfactants in the preparation of formulations. Surfactants can be classified into different classes based on the nature of the hydrophilic group: nonionic, anionic, cationic and amphoteric (see e.g., Ansel's Pharmaceutical Dosage Forms and Drug Delivery Systems, Allen, L V., Popovich N G., and Ansel H C., 2004, Lippincott Williams & Wilkins (8th ed.), New York, NY Rieger, in Pharmaceutical Dosage Forms, Lieberman, Rieger and Banker (Eds.), 1988, Marcel Dekker, Inc., New York, N.Y., volume 1, p. 285).

Naturally occurring emulsifiers used in emulsion formulations include lanolin, beeswax, phosphatides, lecithin and acacia. Absorption bases possess hydrophilic properties such that they can soak up water to form w/o emulsions yet retain their semisolid consistencies, such as anhydrous lanolin and hydrophilic petrolatum. Finely divided solids have also been used as good emulsifiers especially in combination with surfactants and in viscous preparations. These include polar inorganic solids, such as heavy metal hydroxides, nonswelling clays such as bentonite, attapulgite, hectorite, kaolin, montmorillonite, colloidal aluminum silicate and colloidal magnesium aluminum silicate, pigments and nonpolar solids such as carbon or glyceryl tristearate.

A large variety of non-emulsifying materials are also included in emulsion formulations and contribute to the properties of emulsions. These include fats, oils, waxes, fatty acids, fatty alcohols, fatty esters, humectants, hydrophilic colloids, preservatives and antioxidants (Block, in Pharmaceutical Dosage Forms, Lieberman, Rieger and Banker (Eds.), 1988, Marcel Dekker, Inc., New York, N.Y., volume 1, p. 335; Idson, in Pharmaceutical Dosage Forms, Lieberman, Rieger and Banker (Eds.), 1988, Marcel Dekker, Inc., New York, N.Y., volume 1, p. 199).

Hydrophilic colloids or hydrocolloids include naturally occurring gums and synthetic polymers such as polysaccharides (for example, acacia, agar, alginic acid, carrageenan, guar gum, karaya gum, and tragacanth), cellulose derivatives (for example, carboxymethylcellulose and carboxypropylcellulose), and synthetic polymers (for example, carbomers, cellulose ethers, and carboxyvinyl polymers). These disperse or swell in water to form colloidal solutions that stabilize emulsions by forming strong interfacial films around the dispersed-phase droplets and by increasing the viscosity of the external phase.

Since emulsions often contain a number of ingredients such as carbohydrates, proteins, sterols and phosphatides that can readily support the growth of microbes, these formulations often incorporate preservatives. Commonly used preservatives included in emulsion formulations include methyl paraben, propyl paraben, quaternary ammonium salts, benzalkonium chloride, esters of p-hydroxybenzoic acid, and boric acid. Antioxidants are also commonly added to emulsion formulations to prevent deterioration of the formulation. Antioxidants used can be free radical scavengers such as tocopherols, alkyl gallates, butylated hydroxyanisole, butylated hydroxytoluene, or reducing agents such as ascorbic acid and sodium metabisulfite, and antioxidant synergists such as citric acid, tartaric acid, and lecithin.

The application of emulsion formulations via dermatological, oral and parenteral routes and methods for their manufacture have been reviewed in the literature (see e.g., Ansel's Pharmaceutical Dosage Forms and Drug Delivery Systems, Allen, L V., Popovich N G., and Ansel H C., 2004, Lippincott Williams & Wilkins (8th ed.), New York, NY; Idson, in Pharmaceutical Dosage Forms, Lieberman, Rieger and Banker (Eds.), 1988, Marcel Dekker, Inc., New York, N.Y., volume 1, p. 199). Emulsion formulations for oral delivery have been very widely used because of ease of formulation, as well as efficacy from an absorption and bioavailability standpoint (see e.g., Ansel's Pharmaceutical Dosage Forms and Drug Delivery Systems, Allen, L V., Popovich N G., and Ansel H C., 2004, Lippincott Williams & Wilkins (8th ed.), New York, NY; Rosoff, in Pharmaceutical Dosage Forms, Lieberman, Rieger and Banker (Eds.), 1988, Marcel Dekker, Inc., New York, N.Y., volume 1, p. 245; Idson, in Pharmaceutical Dosage Forms, Lieberman, Rieger and Banker (Eds.), 1988, Marcel Dekker, Inc., New York, N.Y., volume 1, p. 199). Mineral-oil base laxatives, oil-soluble vitamins and high fat nutritive preparations are among the materials that have commonly been administered orally as o/w emulsions.

ii. Microemulsions

In one embodiment of the present invention, the compositions of iRNAs and nucleic acids are formulated as microemulsions. A microemulsion can be defined as a system of water, oil and amphiphile which is a single optically isotropic and thermodynamically stable liquid solution (see e.g., Ansel's Pharmaceutical Dosage Forms and Drug Delivery Systems, Allen, L V., Popovich N G., and Ansel H C., 2004, Lippincott Williams & Wilkins (8th ed.), New York, NY; Rosoff, in Pharmaceutical Dosage Forms, Lieberman, Rieger and Banker (Eds.), 1988, Marcel Dekker, Inc., New York, N.Y., volume 1, p. 245). Typically microemulsions are systems that are prepared by first dispersing an oil in an aqueous surfactant solution and then adding a sufficient amount of a fourth component, generally an intermediate chain-length alcohol to form a transparent system. Therefore, microemulsions have also been described as thermodynamically stable, isotropically clear dispersions of two immiscible liquids that are stabilized by interfacial films of surface-active molecules (Leung and Shah, in: Controlled Release of Drugs: Polymers and Aggregate Systems, Rosoff, M., Ed., 1989, VCH Publishers, New York, pages 185-215). Microemulsions commonly are prepared via a combination of three to five components that include oil, water, surfactant, cosurfactant and electrolyte. Whether the microemulsion is of the water-in-oil (w/o) or an oil-in-water (o/w) type is dependent on the properties of the oil and surfactant used and on the structure and geometric packing of the polar heads and hydrocarbon tails of the surfactant molecules (Schott, in Remington's Pharmaceutical Sciences, Mack Publishing Co., Easton, Pa., 1985, p. 271).

The phenomenological approach utilizing phase diagrams has been extensively studied and has yielded a comprehensive knowledge, to one skilled in the art, of how to formulate microemulsions (see e.g., Ansel's Pharmaceutical Dosage Forms and Drug Delivery Systems, Allen, L V., Popovich N G., and Ansel H C., 2004, Lippincott Williams & Wilkins (8th ed.), New York, NY; Rosoff, in Pharmaceutical Dosage Forms, Lieberman, Rieger and Banker (Eds.), 1988, Marcel Dekker, Inc., New York, N.Y., volume 1, p. 245; Block, in Pharmaceutical Dosage Forms, Lieberman, Rieger and Banker (Eds.), 1988, Marcel Dekker, Inc., New York, N.Y., volume 1, p. 335). Compared to conventional emulsions, microemulsions offer the advantage of solubilizing water-insoluble drugs in a formulation of thermodynamically stable droplets that are formed spontaneously.

Surfactants used in the preparation of microemulsions include, but are not limited to, ionic surfactants, non-ionic surfactants, Brij 96, polyoxyethylene oleyl ethers, polyglycerol fatty acid esters, tetraglycerol monolaurate (ML310), tetraglycerol monooleate (MO310), hexaglycerol monooleate (PO310), hexaglycerol pentaoleate (PO500), decaglycerol monocaprate (MCA750), decaglycerol monooleate (MO750), decaglycerol sesquioleate (SO750), decaglycerol decaoleate (DAO750), alone or in combination with cosurfactants. The cosurfactant, usually a short-chain alcohol such as ethanol, 1-propanol, and 1-butanol, serves to increase the interfacial fluidity by penetrating into the surfactant film and consequently creating a disordered film because of the void space generated among surfactant molecules. Microemulsions can, however, be prepared without the use of cosurfactants and alcohol-free self-emulsifying microemulsion systems are known in the art. The aqueous phase can typically be, but is not limited to, water, an aqueous solution of the drug, glycerol, PEG300, PEG400, polyglycerols, propylene glycols, and derivatives of ethylene glycol. The oil phase can include, but is not limited to, materials such as Captex 300, Captex 355, Capmul MCM, fatty acid esters, medium chain (C8-C12) mono, di, and tri-glycerides, polyoxyethylated glyceryl fatty acid esters, fatty alcohols, polyglycolized glycerides, saturated polyglycolized C8-C10 glycerides, vegetable oils and silicone oil.

Microemulsions are particularly of interest from the standpoint of drug solubilization and the enhanced absorption of drugs. Lipid based microemulsions (both o/w and w/o) have been proposed to enhance the oral bioavailability of drugs, including peptides (see e.g., U.S. Pat. Nos. 6,191,105; 7,063,860; 7,070,802; 7,157,099; Constantinides et al., Pharmaceutical Research, 1994, 11, 1385-1390; Ritschel, Meth. Find. Exp. Clin. Pharmacol., 1993, 13, 205). Microemulsions afford advantages of improved drug solubilization, protection of drug from enzymatic hydrolysis, possible enhancement of drug absorption due to surfactant-induced alterations in membrane fluidity and permeability, ease of preparation, ease of oral administration over solid dosage forms, improved clinical potency, and decreased toxicity (see e.g., U.S. Pat. Nos. 6,191,105; 7,063,860; 7,070,802; 7,157,099; Constantinides et al., Pharmaceutical Research, 1994, 11, 1385; Ho et al., J. Pharm. Sci., 1996, 85, 138-143). Often microemulsions can form spontaneously when their components are brought together at ambient temperature. This can be particularly advantageous when formulating thermolabile drugs, peptides or iRNAs. Microemulsions have also been effective in the transdermal delivery of active components in both cosmetic and pharmaceutical applications. It is expected that the microemulsion compositions and formulations of the present invention will facilitate the increased systemic absorption of iRNAs and nucleic acids from the gastrointestinal tract, as well as improve the local cellular uptake of iRNAs and nucleic acids.

Microemulsions of the present invention can also contain additional components and additives such as sorbitan monostearate (Grill 3), Labrasol, and penetration enhancers to improve the properties of the formulation and to enhance the absorption of the iRNAs and nucleic acids of the present invention. Penetration enhancers used in the microemulsions of the present invention can be classified as belonging to one of five broad categories—surfactants, fatty acids, bile salts, chelating agents, and non-chelating non-surfactants (Lee et al., Critical Reviews in Therapeutic Drug Carrier Systems, 1991, p. 92). Each of these classes has been discussed above.

iii. Microparticles

A nucleic acid inhibitor of the invention may be incorporated into a particle, e.g., a microparticle. Microparticles can be produced by spray-drying, but may also be produced by other methods including lyophilization, evaporation, fluid bed drying, vacuum drying, or a combination of these techniques.

iv. Penetration Enhancers

In one embodiment, the present invention employs various penetration enhancers to effect the efficient delivery of nucleic acids, particularly iRNAs, to the skin of animals. Most drugs are present in solution in both ionized and nonionized forms. However, usually only lipid soluble or lipophilic drugs readily cross cell membranes. It has been discovered that even non-lipophilic drugs can cross cell membranes if the membrane to be crossed is treated with a penetration enhancer. In addition to aiding the diffusion of non-lipophilic drugs across cell membranes, penetration enhancers also enhance the permeability of lipophilic drugs.

Penetration enhancers can be classified as belonging to one of five broad categories, i.e., surfactants, fatty acids, bile salts, chelating agents, and non-chelating non-surfactants (see e.g., Malmsten, M. Surfactants and polymers in drug delivery, Informa Health Care, New York, NY, 2002; Lee et al., Critical Reviews in Therapeutic Drug Carrier Systems, 1991, p. 92). Each of the above mentioned classes of penetration enhancers are described below in greater detail.

Surfactants (or “surface-active agents”) are chemical entities which, when dissolved in an aqueous solution, reduce the surface tension of the solution or the interfacial tension between the aqueous solution and another liquid, with the result that absorption of iRNAs through the mucosa is enhanced. In addition to bile salts and fatty acids, these penetration enhancers include, for example, sodium lauryl sulfate, polyoxyethylene-9-lauryl ether and polyoxyethylene-20-cetyl ether) (see e.g., Malmsten, M. Surfactants and polymers in drug delivery, Informa Health Care, New York, NY, 2002; Lee et al., Critical Reviews in Therapeutic Drug Carrier Systems, 1991, p. 92); and perfluorochemical emulsions, such as FC-43. Takahashi et al., J. Pharm. Pharmacol., 1988, 40, 252).

Various fatty acids and their derivatives which act as penetration enhancers include, for example, oleic acid, lauric acid, capric acid (n-decanoic acid), myristic acid, palmitic acid, stearic acid, linoleic acid, linolenic acid, dicaprate, tricaprate, monoolein (1-monooleoyl-rac-glycerol), dilaurin, caprylic acid, arachidonic acid, glycerol 1-monocaprate, 1-dodecylazacycloheptan-2-one, acylcarnitines, acylcholines, C₁₋₂₀ alkyl esters thereof (e.g., methyl, isopropyl and t-butyl), and mono- and di-glycerides thereof (i.e., oleate, laurate, caprate, myristate, palmitate, stearate, linoleate, etc.) (see e.g., Touitou, E., et al. Enhancement in Drug Delivery, CRC Press, Danvers, M A, 2006; Lee et al., Critical Reviews in Therapeutic Drug Carrier Systems, 1991, p. 92; Muranishi, Critical Reviews in Therapeutic Drug Carrier Systems, 1990, 7, 1-33; El Hariri et al., J. Pharm. Pharmacol., 1992, 44, 651-654).

The physiological role of bile includes the facilitation of dispersion and absorption of lipids and fat-soluble vitamins (see e.g., Malmsten, M. Surfactants and polymers in drug delivery, Informa Health Care, New York, NY, 2002; Brunton, Chapter 38 in: Goodman & Gilman's The Pharmacological Basis of Therapeutics, 9th Ed., Hardman et al. Eds., McGraw-Hill, New York, 1996, pp. 934-935). Various natural bile salts, and their synthetic derivatives, act as penetration enhancers. Thus the term “bile salts” includes any of the naturally occurring components of bile as well as any of their synthetic derivatives. Suitable bile salts include, for example, cholic acid (or its pharmaceutically acceptable sodium salt, sodium cholate), dehydrocholic acid (sodium dehydrocholate), deoxycholic acid (sodium deoxycholate), glucholic acid (sodium glucholate), glycholic acid (sodium glycocholate), glycodeoxycholic acid (sodium glycodeoxycholate), taurocholic acid (sodium taurocholate), taurodeoxycholic acid (sodium taurodeoxycholate), chenodeoxycholic acid (sodium chenodeoxycholate), ursodeoxycholic acid (UDCA), sodium tauro-24,25-dihydro-fusidate (STDHF), sodium glycodihydrofusidate and polyoxyethylene-9-lauryl ether (POE) (see e.g., Malmsten, M. Surfactants and polymers in drug delivery, Informa Health Care, New York, NY, 2002; Lee et al., Critical Reviews in Therapeutic Drug Carrier Systems, 1991, page 92; Swinyard, Chapter 39 In: Remington's Pharmaceutical Sciences, 18th Ed., Gennaro, ed., Mack Publishing Co., Easton, Pa., 1990, pages 782-783; Muranishi, Critical Reviews in Therapeutic Drug Carrier Systems, 1990, 7, 1-33; Yamamoto et al., J. Pharm. Exp. Ther., 1992, 263, 25; Yamashita et al., J. Pharm. Sci., 1990, 79, 579-583).

Chelating agents, as used in connection with the present invention, can be defined as compounds that remove metallic ions from solution by forming complexes therewith, with the result that absorption of nucleic acid inhibitors s through the mucosa is enhanced. With regards to their use as penetration enhancers in the present invention, chelating agents have the added advantage of also serving as DNase inhibitors, as most characterized DNA nucleases require a divalent metal ion for catalysis and are thus inhibited by chelating agents (Jarrett, J. Chromatogr., 1993, 618, 315-339). Suitable chelating agents include but are not limited to disodium ethylenediaminetetraacetate (EDTA), citric acid, salicylates (e.g., sodium salicylate, 5-methoxysalicylate and homovanilate), N-acyl derivatives of collagen, laureth-9 and N-amino acyl derivatives of beta-diketones (enamines)(see e.g., Katdare, A. et al., Excipient development for pharmaceutical, biotechnology, and drug delivery, CRC Press, Danvers, M A, 2006; Lee et al., Critical Reviews in Therapeutic Drug Carrier Systems, 1991, page 92; Muranishi, Critical Reviews in Therapeutic Drug Carrier Systems, 1990, 7, 1-33; Buur et al., J. Control Rel., 1990, 14, 43-51).

As used herein, non-chelating non-surfactant penetration enhancing compounds can be defined as compounds that demonstrate insignificant activity as chelating agents or as surfactants but that nonetheless enhance absorption of iRNAs through the alimentary mucosa (see e.g., Muranishi, Critical Reviews in Therapeutic Drug Carrier Systems, 1990, 7, 1-33). This class of penetration enhancers includes, for example, unsaturated cyclic ureas, 1-alkyl- and 1-alkenylazacyclo-alkanone derivatives (Lee et al., Critical Reviews in Therapeutic Drug Carrier Systems, 1991, page 92); and non-steroidal anti-inflammatory agents such as diclofenac sodium, indomethacin and phenylbutazone (Yamashita et al., J. Pharm. Pharmacol., 1987, 39, 621-626).

Agents that enhance uptake of nucleic acid inhibitors at the cellular level can also be added to the pharmaceutical and other compositions of the present invention. For example, cationic lipids, such as lipofectin (Junichi et al, U.S. Pat. No. 5,705,188), cationic glycerol derivatives, and polycationic molecules, such as polylysine (Lollo et al., PCT Application WO 97/30731), are also known to enhance the cellular uptake of dsRNAs. Examples of commercially available transfection reagents include, for example Lipofectamine™ (Invitrogen; Carlsbad, CA), Lipofectamine 2000™ (Invitrogen; Carlsbad, CA), 293Fectin™ (Invitrogen; Carlsbad, CA), Cellfectin™ (Invitrogen; Carlsbad, CA), DMRIE-C™ (Invitrogen; Carlsbad, CA), FreeStyle™ MAX (Invitrogen; Carlsbad, CA), Lipofectamine™ 2000 CD (Invitrogen; Carlsbad, CA), Lipofectamine™ (Invitrogen; Carlsbad, CA), RNAiMAX (Invitrogen; Carlsbad, CA), Oligofectamine™ (Invitrogen; Carlsbad, CA), Optifect™ (Invitrogen; Carlsbad, CA), X-tremeGENE Q2 Transfection Reagent (Roche; Grenzacherstrasse, Switzerland), DOTAP Liposomal Transfection Reagent (Grenzacherstrasse, Switzerland), DOSPER Liposomal Transfection Reagent (Grenzacherstrasse, Switzerland), or Fugene (Grenzacherstrasse, Switzerland), Transfectam® Reagent (Promega; Madison, WI), TransFast™ Transfection Reagent (Promega; Madison, WI), Tfx™-20 Reagent (Promega; Madison, WI), Tfx™-50 Reagent (Promega; Madison, WI), DreamFect™ (OZ Biosciences; Marseille, France), EcoTransfect (OZ Biosciences; Marseille, France), TransPass^{a} D1 Transfection Reagent (New England Biolabs; Ipswich, MA, USA), LyoVec™/LipoGen™ (Invitrogen; San Diego, CA, USA), PerFectin Transfection Reagent (Genlantis; San Diego, CA, USA), NeuroPORTER Transfection Reagent (Genlantis; San Diego, CA, USA), GenePORTER Transfection reagent (Genlantis; San Diego, CA, USA), GenePORTER 2 Transfection reagent (Genlantis; San Diego, CA, USA), Cytofectin Transfection Reagent (Genlantis; San Diego, CA, USA), BaculoPORTER Transfection Reagent (Genlantis; San Diego, CA, USA), TroganPORTER™ transfection Reagent (Genlantis; San Diego, CA, USA), RiboFect (Bioline; Taunton, MA, USA), PlasFect (Bioline; Taunton, MA, USA), UniFECTOR (B-Bridge International; Mountain View, CA, USA), SureFECTOR (B-Bridge International; Mountain View, CA, USA), or HiFect™ (B-Bridge International, Mountain View, CA, USA), among others.

Other agents can be utilized to enhance the penetration of the administered nucleic acids, including glycols such as ethylene glycol and propylene glycol, pyrrols such as 2-pyrrol, azones, and terpenes such as limonene and menthone.

v. Carriers

Certain compositions of the present invention also incorporate carrier compounds in the formulation. As used herein, “carrier compound” or “carrier” can refer to a nucleic acid, or analog thereof, which is inert (i.e., does not possess biological activity per se) but is recognized as a nucleic acid by in vivo processes that reduce the bioavailability of a nucleic acid having biological activity by, for example, degrading the biologically active nucleic acid or promoting its removal from circulation. The coadministration of a nucleic acid and a carrier compound, typically with an excess of the latter substance, can result in a substantial reduction of the amount of nucleic acid recovered in the liver, kidney or other extracirculatory reservoirs, presumably due to competition between the carrier compound and the nucleic acid for a common receptor. For example, the recovery of a partially phosphorothioate dsRNA in hepatic tissue can be reduced when it is coadministered with polyinosinic acid, dextran sulfate, polycytidic acid or 4-acetamido-4′isothiocyano-stilbene-2,2′-disulfonic acid (Miyao et al., DsRNA Res. Dev., 1995, 5, 115-121; Takakura et al., DsRNA & Nucl. Acid Drug Dev., 1996, 6, 177-183.

vi. Excipients

In contrast to a carrier compound, a “pharmaceutical carrier” or “excipient” is a pharmaceutically acceptable solvent, suspending agent or any other pharmacologically inert vehicle for delivering one or more nucleic acids to an animal. The excipient can be liquid or solid and is selected, with the planned manner of administration in mind, so as to provide for the desired bulk, consistency, etc., when combined with a nucleic acid and the other components of a given pharmaceutical composition. Typical pharmaceutical carriers include, but are not limited to, binding agents (e.g., pregelatinized maize starch, polyvinylpyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrants (e.g., starch, sodium starch glycolate, etc.); and wetting agents (e.g., sodium lauryl sulphate, etc). Pharmaceutically acceptable organic or inorganic excipients suitable for non-parenteral administration which do not deleteriously react with nucleic acids can also be used to formulate the compositions of the present invention. Suitable pharmaceutically acceptable carriers include, but are not limited to, water, salt solutions, alcohols, polyethylene glycols, gelatin, lactose, amylose, magnesium stearate, talc, silicic acid, viscous paraffin, hydroxymethylcellulose, polyvinylpyrrolidone and the like.

Formulations for topical administration of nucleic acid inhibitors can include sterile and non-sterile aqueous solutions, non-aqueous solutions in common solvents such as alcohols, or solutions of the nucleic acids in liquid or solid oil bases. The solutions can also contain buffers, diluents and other suitable additives. Pharmaceutically acceptable organic or inorganic excipients suitable for non-parenteral administration which do not deleteriously react with nucleic acids can be used. Suitable pharmaceutically acceptable excipients include, but are not limited to, water, salt solutions, alcohol, polyethylene glycols, gelatin, lactose, amylose, magnesium stearate, talc, silicic acid, viscous paraffin, hydroxymethylcellulose, polyvinylpyrrolidone and the like.

vii. Other Components

The compositions of the present invention can additionally contain other adjunct components conventionally found in pharmaceutical compositions, at their art-established usage levels. Thus, for example, the compositions can contain additional, compatible, pharmaceutically-active materials such as, for example, antipruritics, astringents, local anesthetics or anti-inflammatory agents, or can contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Aqueous suspensions can contain substances which increase the viscosity of the suspension including, for example, sodium carboxymethylcellulose, sorbitol and/or dextran. The suspension can also contain stabilizers.

In some embodiments, pharmaceutical compositions featured in the invention include (a) one or more nucleic acid inhibitors and (b) one or more agents which function by a non-RNAi mechanism and which are useful in treating a kidney stone disease. Examples of such agents include, but are not limited to pyridoxine, an ACE inhibitor (angiotensin converting enzyme inhibitors), e.g., benazepril (Lotensin); an angiotensin II receptor antagonist (ARB) (e.g., losartan potassium, such as Merck & Co.'s Cozaar®), e.g., Candesartan (Atacand); an HMG-CoA reductase inhibitor (e.g., a statin); dietary oxalate degrading compounds, e.g., Oxalate decarboxylase (Oxazyme); calcium binding agents, e.g., Sodium cellulose phosphate (Calcibind); diuretics, e.g., thiazide diuretics, such as hydrochlorothiazide (Microzide); phosphate binders, e.g., Sevelamer (Renagel); magnesium and Vitamin B6 supplements; potassium citrate; orthophosphates, bisphosphonates; oral phosphate and citrate solutions; high fluid intake, urinary tract endoscopy; extracorporeal shock wave lithotripsy; kidney dialysis; kidney stone removal (e.g., surgery); and kidney/liver transplant; or a combination of any of the foregoing.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit high therapeutic indices are preferred. The data obtained from cell culture assays and animal studies can be used in formulating a range of dosage for use in humans. The dosage of compositions featured herein in the invention lies generally within a range of circulating concentrations that include the ED₅₀ with little or no toxicity. The dosage can vary within this range depending upon the dosage form employed and the route of administration utilized. For any compound used in the methods featured in the invention, the therapeutically effective dose can be estimated initially from cell culture assays. A dose can be formulated in animal models to achieve a circulating plasma concentration range of the compound or, when appropriate, of the polypeptide product of a target sequence (e.g., achieving a decreased concentration of the polypeptide) that includes the IC₅₀ (i.e., the concentration of the test compound which achieves a half-maximal inhibition of symptoms) as determined in cell culture. Such information can be used to more accurately determine useful doses in humans. Levels in plasma can be measured, for example, by high performance liquid chromatography.

### VII. Kits of the Invention

In certain aspects, the instant disclosure provides kits that include a suitable container containing a pharmaceutical formulation of a nucleic acid inhibitor. In certain embodiments the individual components of the pharmaceutical formulation may be provided in one container. Alternatively, it may be desirable to provide the components of the pharmaceutical formulation separately in two or more containers, e.g., one container for a nucleic acid inhibitor preparation, and at least another for a carrier compound. The kit may be packaged in a number of different configurations such as one or more containers in a single box. The different components can be combined, e.g., according to instructions provided with the kit. The components can be combined according to a method described herein, e.g., to prepare and administer a pharmaceutical composition. The kit can also include a delivery device.

Unless otherwise defined, all technical and scientific terms used herein have the same meaning as commonly understood by one of ordinary skill in the art to which this invention belongs. Although methods and materials similar or equivalent to those described herein can be used in the practice or testing of the RNAi agents and methods featured in the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

A Sequence Listing is filed herewith and forms part of the specification as filed. A supplemental informal sequence listing is also included as part of the specification.

### EXAMPLES

Example 1. Treating Subjects Having or at Risk of Developing a Non-Primary Hyperoxaluria Disease with a Nucleic Acid Inhibitor of LDHA, a Nucleic Acid Inhibitor of PRODH2 and/or a Nucleic Acid Inhibitor of HAO1

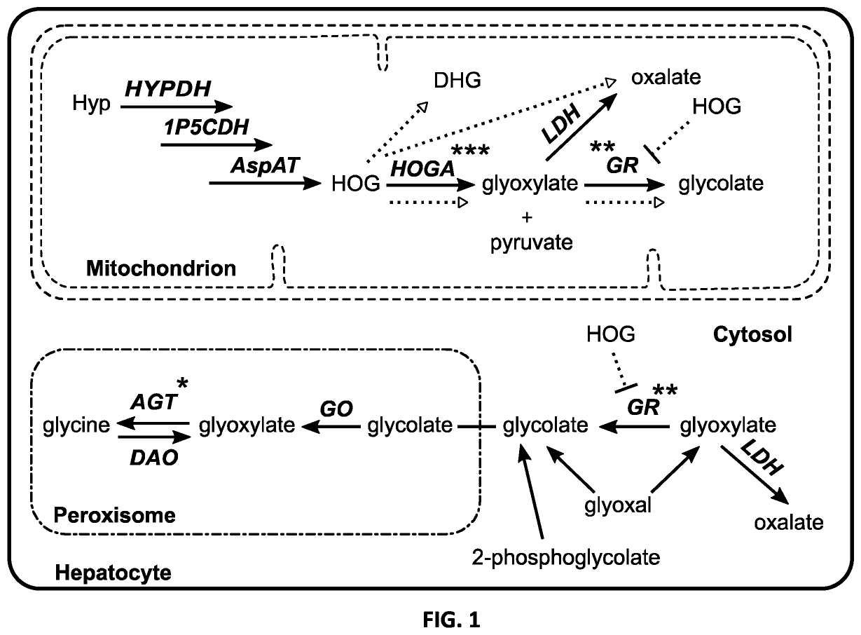

Primary hyperoxaluria is a well-known disease associated with high levels of oxalate. Specifically, primary hyperoxaluria is characterized by impaired glyoxylate metabolism resulting in overproduction and accumulation of oxalate throughout the body, typically manifesting as kidney and bladder stones. There are three major types of primary hyperoxaluria that differ in their severity and genetic cause. Autosomal recessive mutations in the AGXT gene cause primary hyperoxaluria type 1 (PH1); autosomal recessive mutations in the GRHPR gene cause primary hyperoxaluria type 2 (PH2); and autosomal recessive mutations in the HOGA1 gene cause primary hyperoxaluria type 3 (PH3) (see, FIG. 1).

Therapeutics that reduce oxalate levels have entered the clinic for the treatment of subjects having PH1 and PH2. Specifically, Lumasiran, an RNA interference (RNAi) therapeutic targeting glycolate oxidase (GO) for treatment of PH1 is currently being evaluated in a Phase III clinical trail (see, e.g., NCT03681184), and DCR-PHXC, an RNA interference (RNAi) therapeutic targeting LDHA for the treatment of PH1 and PH2 has entered Phase II clinical trials (see, e.g., NCT03847909).

However, there are also a significant number of subjects that do not have primary hyperoxaluria, e.g., PH1, PH2, or PH3, but yet still would benefit from treatment with agents that reduce oxalate. For example, subjects having or at risk of developing a non-primary hyperoxaluria disease or disorder, as described herein, would benefit from treatment with agents that reduce oxalate.

Specifically, subjects having a non-primary hyperoxaluria disease or disorder that would benefit from reduction in oxalate include subjects having elevated levels of oxalate who suffer from enteric hyperoxaluria, dietary hyperoxaluria, idiopathic hyperoxaluria, a kidney stone disease, chronic kidney disease (CKD), end-stage renal disease (ESRD), coronary artery disease, cutaneous oxalate deposition, or ethylene glycol poisoning, or those who are planning to undergo kidney transplantation or have undergone kidney transplantation. Subjects having a non-primary hyperoxaluria disease or disorder do not have primary hyperoxaluria (PH), i.e., PH1, PH2, or PH3.

Subjects at risk of developing a non-primary hyperoxaluria disease or disorder that would benefit from reduction in oxalate include subjects having normal levels of oxalate who suffer from a kidney stone disease, end-stage renal disease (ESRD), coronary artery disease, diabetes, cutaneous oxalate deposition, or ethylene glycol poisoning. Subjects at risk of developing a non-primary hyperoxaluria disease or disorder do not have primary hyperoxaluria (PH), i.e., PH1, PH2, or PH3.

Accordingly, the present invention provides methods for treating subjects having or at risk of developing a non-primary hyperoxaluria disease or disorder that would benefit from reduction in oxalate using nucleic acid inhibitors, e.g., double stranded ribonucleic acid (dsRNA) agents or single stranded antisense polynucleotide agents targeting lactate dehydrogenase A (LDHA), hydroxyacid oxidase (HAO1) and/or proline dehydrogenase 2 (PRODH2), as described herein.

Tables 2-16 provide exemplary nucleic acid inhibitors for LDHA, HAO1 and/or PRODH2 for use in the methods of the present invention.

TABLE 1
Abbreviations of nucleotide monomers used in nucleic acid sequence representation.
It will be understood that these monomers, when present in an oligonucleotide, are mutually linked by
5′-3′-phosphodiester bonds; and it is understood that when the nucleotide contains a 2′-fluoro
modification, then the fluoro replaces the hydroxy at that position in the parent nucleotide (i.e., it is a
2′-deoxy-2′-fluoronucleotide).

| Abbreviation | Nucleotide(s) |
|---|---|
| A | Adenosine-3′-phosphate |
| Ab | beta-L-adenosine-3′-phosphate |
| Abs | beta-L-adenosine-3′-phosphorothioate |
| Af | 2′-fluoroadenosine-3′-phosphate |
| Afs | 2′-fluoroadenosine-3′-phosphorothioate |
| As | adenosine-3′-phosphorothioate |
| C | cytidine-3′-phosphate |
| Cb | beta-L-cytidine-3′-phosphate |
| Cbs | beta-L-cytidine-3′-phosphorothioate |
| Cf | 2′-fluorocytidine-3′-phosphate |
| Cfs | 2′-fluorocytidine-3′-phosphorothioate |
| Cs | cytidine-3′-phosphorothioate |
| G | guanosine-3′-phosphate |
| Gb | beta-L-guanosine-3′-phosphate |
| Gbs | beta-L-guanosine-3′-phosphorothioate |
| Gf | 2′-fluoroguanosine-3′-phosphate |
| Gfs | 2′-fluoroguanosine-3′-phosphorothioate |
| Gs | guanosine-3′-phosphorothioate |
| T | 5′-methyluridine-3′-phosphate |
| Tf | 2′-fluoro-5-methyluridine-3′-phosphate |
| Tfs | 2′-fluoro-5-methyluridine-3′-phosphorothioate |
| Ts | 5-methyluridine-3′-phosphorothioate |
| U | Uridine-3′-phosphate |
| Uf | 2′-fluorouridine-3′-phosphate |
| Ufs | 2′-fluorouridine-3′-phosphorothioate |
| Us | uridine-3′-phosphorothioate |
| N | any nucleotide, modified or unmodified |
| a | 2′-O-methyladenosine-3′-phosphate |
| as | 2′-O-methyladenosine-3′-phosphorothioate |
| c | 2′-O-methylcytidine-3′-phosphate |
| cs | 2′-O-methylcytidine-3′-phosphorothioate |
| g | 2′-O-methylguanosine-3′-phosphate |
| gs | 2′-O-methylguanosine-3′-phosphorothioate |
| t | 2′-O-methyl-5-methyluridine-3′-phosphate |
| ts | 2′-O-methyl-5-methyluridine-3′-phosphorothioate |
| u | 2′-O-methyluridine-3′-phosphate |
| us | 2′-O-methyluridine-3′-phosphorothioate |
| s | phosphorothioate linkage |
| L96 | N-[tris(GalNAc-alkyl)-amidodecanoyl)]-4-hydroxyprolinol |
| | (Hyp-(GalNAc-alkyl)3) |
| | 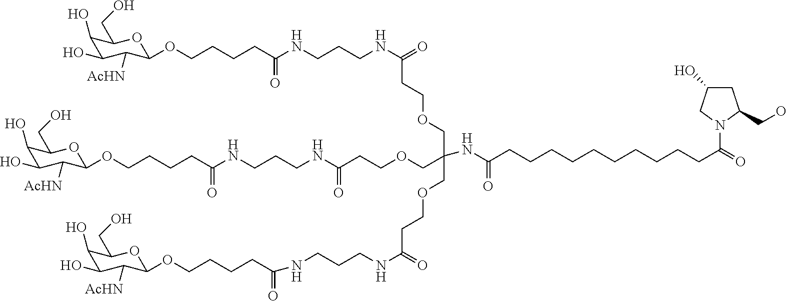 [SMILES: CC(=O)NC1C(OCCCCC(=O)NCCCNC(=O)CCOCC(COCCC(=O)NCCCNC(=O)CCCCOC2OC(CO)C(O)C(O)C2NC(C)=O)(COCCC(=O)NCCCNC(=O)CCCCOC2OC(CO)C(O)C(O)C2NC(C)=O)NC(=O)CCCCCCCCCCC(=O)N2C[C@H](O)C[C@H]2CO)OC(CO)C(O)C1O] |
| Y34 | 2-hydroxymethyl-tetrahydrofurane-4-methoxy-3-phosphate (abasic 2′-OMe |
| | furanose) |
| | 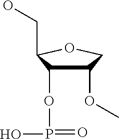 |
| Y44 | inverted abasic DNA (2-hydroxymethyl-tetrahydrofurane-5-phosphate) |
| | 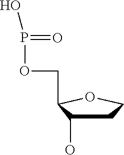 |
| L10 | N-(cholesterylcarboxamidocaproyl)-4-hydroxyprolinol (Hyp-C6-Chol) |
| | 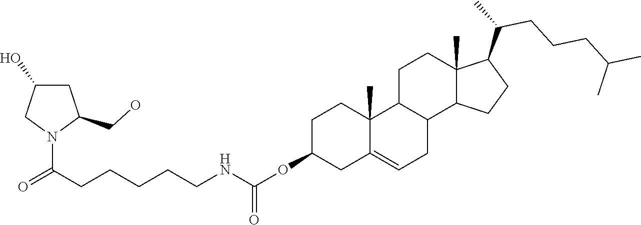 [SMILES: CC(C)CCC[C@@H](C)[C@H]1CCC2C3CC=C4C[C@@H](OC(=O)NCCCCCC(=O)N5C[C@H](O)C[C@H]5CO)CC[C@]4(C)C3CC[C@@]21C] |
| (Agn) | Adenosine-glycol nucleic acid (GNA) S-Isomer |
| (Cgn) | Cytidine-glycol nucleic acid (GNA) S-Isomer |
| (Ggn) | Guanosine-glycol nucleic acid (GNA) S-Isomer |
| (Tgn) | Thymidine-glycol nucleic acid (GNA) S-Isomer |
| P | Phosphate |
| VP | Vinyl-phosphonate |
| dA | 2′-deoxyadenosine-3′-phosphate |
| dAs | 2′-deoxyadenosine-3′-phosphorothioate |
| dC | 2′-deoxycytidine-3′-phosphate |
| dCs | 2′-deoxycytidine-3′-phosphorothioate |
| dG | 2′-deoxyguanosine-3′-phosphate |
| dGs | 2′-deoxyguanosine-3′-phosphorothioate |
| dT | 2′-deoxythymidine-3′-phosphate |
| dTs | 2′-deoxythymidine-3′-phosphorothioate |
| dU | 2′-deoxyuridine |
| dUs | 2′-deoxyuridine-3′-phosphorothioate |
| (C2p) | cytidine-2′-phosphate |
| (G2p) | guanosine-2′-phosphate |
| (U2p) | uridine-2′-phosphate |
| (A2p) | adenosine-2′-phosphate |
| (Ahd) | 2′-O-hexadecyl-adenosine-3′-phosphate |
| (Ahds) | 2′-O-hexadecyl-adenosine-3′-phosphorothioate |
| (Chd) | 2′-O-hexadecyl-cytidine-3′-phosphate |
| (Chds) | 2′-O-hexadecyl-cytidine-3′-phosphorothioate |
| (Ghd) | 2′-O-hexadecyl-guanosine-3′-phosphate |
| (Ghds) | 2′-O-hexadecyl-guanosine-3′-phosphorothioate |
| (Uhd) | 2′-O-hexadecyl-uridine-3′-phosphate |
| (Uhds) | 2′-O-hexadecyl-uridine-3′-phosphorothioate |
| s | phosphorothioate |

TABLE 2
UNMODIFIED HUMAN/CYNOMOLGUS CROSS-REACTIVE LDHA IRNA SEQUENCES

| | | SEQ | Position | | SEQ | Position |
|---|---|---|---|---|---|---|
| Duplex | Sense Sequence | ID | in NM_ | Antisense Sequence | ID | in NM_ |
| Name | 5′ to 3′ | NO | 005566.3 | 5′ to 3′ | NO | 005566.3 |
| AD-159469 | UUUAUCUGAUCUGUGAUUAAA | 3210 | 1347-1367 | UUUAAUCACAGAUCAGAUAAAAA | 3396 | 1345-1367 |
| AD-159607 | ACUGGUUAGUGUGAAAUAGUU | 3211 | 1489-1509 | AACUAUUUCACACUAACCAGUUG | 3397 | 1487-1509 |
| AD-159713 | AACAUGCCUAGUCCAACAUUU | 3212 | 1615-1635 | AAAUGUUGGACUAGGCAUGUUCA | 3398 | 1613-1635 |
| AD-158504 | CAAGUCCAAUAUGGCAACUCU | 3213 | 263-283 | AGAGUUGCCAUAUUGGACUUGGA | 3399 | 261-283 |
| AD-159233 | UCCACCAUGAUUAAGGGUCUU | 3214 | 1092-1112 | AAGACCCUUAAUCAUGGUGGAAA | 3400 | 1090-1112 |
| AD-159411 | UCAUUUCACUGUCUAGGCUAA | 3215 | 1289-1309 | UUAGCCUAGACAGUGAAAUGAUA | 3401 | 1287-1309 |
| AD-159462 | UGUCCUUUUUAUCUGAUCUGU | 3216 | 1340-1360 | ACAGAUCAGAUAAAAAGGACAAC | 3402 | 1338-1360 |
| AD-159742 | CCAGUGUAUAAAUCCAAUAUA | 3217 | 1662-1682 | UAUAUUGGAUUUAUACACUGGAU | 3403 | 1660-1682 |
| AD-159863 | UCCAAGUGUUAUACCAACUAA | 3218 | 1791-1811 | UUAGUUGGUAUAACACUUGGAUA | 3404 | 1789-1811 |
| AD-158626 | GUCAUCGAAGACAAAUUGAAA | 3219 | 429-449 | UUUCAAUUUGUCUUCGAUGACAU | 3405 | 427-449 |
| AD-158687 | GAACACCAAAGAUUGUCUCUA | 3220 | 490-510 | UAGAGACAAUCUUUGGUGUUCUA | 3406 | 488-510 |
| AD-158688 | AACACCAAAGAUUGUCUCUGA | 3221 | 491-511 | UCAGAGACAAUCUUUGGUGUUCU | 3407 | 489-511 |
| AD-159458 | AUGUUGUCCUUUUUAUCUGAU | 3222 | 1336-1356 | AUCAGAUAAAAAGGACAACAUGC | 3408 | 1334-1356 |
| AD-159519 | UCAACUCCUGAAGUUAGAAAU | 3223 | 1401-1421 | AUUUCUAACUUCAGGAGUUGAUG | 3409 | 1399-1421 |
| AD-159858 | AACUAUCCAAGUGUUAUACCA | 3224 | 1786-1806 | UGGUAUAACACUUGGAUAGUUGG | 3410 | 1784-1806 |
| AD-158681 | UCCUUAGAACACCAAAGAUUA | 3225 | 484-504 | UAAUCUUUGGUGUUCUAAGGAAA | 3411 | 482-504 |
| AD-159583 | GGUAUUAAUCUUGUGUAGUCU | 3226 | 1465-1485 | AGACUACACAAGAUUAAUACCAU | 3412 | 1463-1485 |
| AD-159700 | GGCUCCUUCACUGAACAUGCA | 3227 | 1602-1622 | UGCAUGUUCAGUGAAGGAGCCAG | 3413 | 1600-1622 |
| AD-159807 | UAUCAGUAGUGUACAUUACCA | 3228 | 1728-1748 | UGGUAAUGUACACUACUGAUAUA | 3414 | 1726-1748 |
| AD-158673 | CAGCCUUUUCCUUAGAACACA | 3229 | 476-496 | UGUGUUCUAAGGAAAAGGCUGCC | 3415 | 474-496 |
| AD-159608 | CUGGUUAGUGUGAAAUAGUUA | 3230 | 1490-1510 | UAACUAUUUCACACUAACCAGUU | 3416 | 1488-1510 |
| AD-159803 | ACUAUAUCAGUAGUGUACAUU | 3231 | 1724-1744 | AAUGUACACUACUGAUAUAGUUC | 3417 | 1722-1744 |
| AD-159805 | UAUAUCAGUAGUGUACAUUAA | 3232 | 1726-1746 | UUAAUGUACACUACUGAUAUAGU | 3418 | 1724-1746 |
| AD-159489 | GUAAUAUUUUAAGAUGGACUA | 3233 | 1371-1391 | UAGUCCAUCUUAAAAUAUUACUG | 3419 | 1369-1391 |
| AD-159495 | UUUUAAGAUGGACUGGGAAAA | 3234 | 1377-1397 | UUUUCCCAGUCCAUCUUAAAAUA | 3420 | 1375-1397 |
| AD-159609 | UGGUUAGUGUGAAAUAGUUCU | 3235 | 1491-1511 | AGAACUAUUUCACACUAACCAGU | 3421 | 1489-1511 |
| AD-159706 | UUCACUGAACAUGCCUAGUCA | 3236 | 1608-1628 | UGACUAGGCAUGUUCAGUGAAGG | 3422 | 1606-1628 |
| AD-159855 | ACCAACUAUCCAAGUGUUAUA | 3237 | 1783-1803 | UAUAACACUUGGAUAGUUGGUUG | 3423 | 1781-1803 |
| AD-159864 | CCAAGUGUUAUACCAACUAAA | 3238 | 1792-1812 | UUUAGUUGGUAUAACACUUGGAU | 3424 | 1790-1812 |
| AD-158491 | UUCCUUUUGGUUCCAAGUCCA | 3239 | 250-270 | UGGACUUGGAACCAAAAGGAAUC | 3425 | 248-270 |
| AD-158672 | GCAGCCUUUUCCUUAGAACAA | 3240 | 475-495 | UUGUUCUAAGGAAAAGGCUGCCA | 3426 | 473-495 |
| AD-159488 | AGUAAUAUUUUAAGAUGGACU | 3241 | 1370-1390 | AGUCCAUCUUAAAAUAUUACUGC | 3427 | 1368-1390 |
| AD-159553 | AAAAUCCACAGCUAUAUCCUA | 3242 | 1435-1455 | UAGGAUAUAGCUGUGGAUUUUAC | 3428 | 1433-1455 |
| AD-159703 | UCCUUCACUGAACAUGCCUAA | 3243 | 1605-1625 | UUAGGCAUGUUCAGUGAAGGAGC | 3429 | 1603-1625 |
| AD-159708 | CACUGAACAUGCCUAGUCCAA | 3244 | 1610-1630 | UUGGACUAGGCAUGUUCAGUGAA | 3430 | 1608-1630 |
| AD-159866 | AAGUGUUAUACCAACUAAAAC | 3245 | 1794-1814 | GUUUUAGUUGGUAUAACACUUGG | 3431 | 1792-1814 |
| AD-159232 | UUCCACCAUGAUUAAGGGUCU | 3246 | 1091-1111 | AGACCCUUAAUCAUGGUGGAAAC | 3432 | 1089-1111 |
| AD-159712 | GAACAUGCCUAGUCCAACAUU | 3247 | 1614-1634 | AAUGUUGGACUAGGCAUGUUCAG | 3433 | 1612-1634 |
| AD-159808 | AUCAGUAGUGUACAUUACCAU | 3248 | 1729-1749 | AUGGUAAUGUACACUACUGAUAU | 3434 | 1727-1749 |
| AD-159862 | AUCCAAGUGUUAUACCAACUA | 3249 | 1790-1810 | UAGUUGGUAUAACACUUGGAUAG | 3435 | 1788-1810 |
| AD-158503 | CCAAGUCCAAUAUGGCAACUA | 3250 | 262-282 | UAGUUGCCAUAUUGGACUUGGAA | 3436 | 260-282 |
| AD-159311 | AUCUCAGACCUUGUGAAGGUA | 3251 | 1170-1190 | UACCUUCACAAGGUCUGAGAUUC | 3437 | 1168-1190 |
| AD-159412 | CAUUUCACUGUCUAGGCUACA | 3252 | 1290-1310 | UGUAGCCUAGACAGUGAAAUGAU | 3438 | 1288-1310 |
| AD-159558 | CCACAGCUAUAUCCUGAUGCU | 3253 | 1440-1460 | AGCAUCAGGAUAUAGCUGUGGAU | 3439 | 1438-1460 |
| AD-159705 | CUUCACUGAACAUGCCUAGUA | 3254 | 1607-1627 | UACUAGGCAUGUUCAGUGAAGGA | 3440 | 1605-1627 |
| AD-159113 | GUGGUUGAGAGUGCUUAUGAA | 3255 | 972-992 | UUCAUAAGCACUCUCAACCACCU | 3441 | 970-992 |
| AD-159139 | CAAACUCAAAGGCUACACAUA | 3256 | 998-1018 | UAUGUGUAGCCUUUGAGUUUGAU | 3442 | 996-1018 |
| AD-159806 | AUAUCAGUAGUGUACAUUACA | 3257 | 1727-1747 | UGUAAUGUACACUACUGAUAUAG | 3443 | 1725-1747 |
| AD-159853 | CAACCAACUAUCCAAGUGUUA | 3258 | 1781-1801 | UAACACUUGGAUAGUUGGUUGCA | 3444 | 1779-1801 |
| AD-158627 | UCAUCGAAGACAAAUUGAAGA | 3259 | 430-450 | UCUUCAAUUUGUCUUCGAUGACA | 3445 | 428-450 |
| AD-159182 | GCAGAUUUGGCAGAGAGUAUA | 3260 | 1041-1061 | UAUACUCUCUGCCAAAUCUGCUA | 3446 | 1039-1061 |
| AD-159702 | CUCCUUCACUGAACAUGCCUA | 3261 | 1604-1624 | UAGGCAUGUUCAGUGAAGGAGCC | 3447 | 1602-1624 |
| AD-159715 | CAUGCCUAGUCCAACAUUUUU | 3262 | 1617-1637 | AAAAAUGUUGGACUAGGCAUGUU | 3448 | 1615-1637 |
| AD-158575 | UGCCAUCAGUAUCUUAAUGAA | 3263 | 377-397 | UUCAUUAAGAUACUGAUGGCACA | 3449 | 375-397 |
| AD-158576 | GCCAUCAGUAUCUUAAUGAAA | 3264 | 378-398 | UUUCAUUAAGAUACUGAUGGCAC | 3450 | 376-398 |
| AD-158684 | UUAGAACACCAAAGAUUGUCU | 3265 | 487-507 | AGACAAUCUUUGGUGUUCUAAGG | 3451 | 485-507 |
| AD-159410 | AUCAUUUCACUGUCUAGGCUA | 3266 | 1288-1308 | UAGCCUAGACAGUGAAAUGAUAU | 3452 | 1286-1308 |
| AD-159416 | UCACUGUCUAGGCUACAACAA | 3267 | 1294-1314 | UUGUUGUAGCCUAGACAGUGAAA | 3453 | 1292-1314 |
| AD-159738 | GGAUCCAGUGUAUAAAUCCAA | 3268 | 1658-1678 | UUGGAUUUAUACACUGGAUCCCA | 3454 | 1656-1678 |
| AD-159857 | CAACUAUCCAAGUGUUAUACA | 3269 | 1785-1805 | UGUAUAACACUUGGAUAGUUGGU | 3455 | 1783-1805 |
| AD-158497 | UUGGUUCCAAGUCCAAUAUGA | 3270 | 256-276 | UCAUAUUGGACUUGGAACCAAAA | 3456 | 254-276 |
| AD-159124 | UGCUUAUGAGGUGAUCAAACU | 3271 | 983-1003 | AGUUUGAUCACCUCAUAAGCACU | 3457 | 981-1003 |
| AD-159140 | AAACUCAAAGGCUACACAUCA | 3272 | 999-1019 | UGAUGUGUAGCCUUUGAGUUUGA | 3458 | 997-1019 |
| AD-159312 | UCUCAGACCUUGUGAAGGUGA | 3273 | 1171-1191 | UCACCUUCACAAGGUCUGAGAUU | 3459 | 1169-1191 |
| AD-159552 | UAAAAUCCACAGCUAUAUCCU | 3274 | 1434-1454 | AGGAUAUAGCUGUGGAUUUUACA | 3460 | 1432-1454 |
| AD-159704 | CCUUCACUGAACAUGCCUAGU | 3275 | 1606-1626 | ACUAGGCAUGUUCAGUGAAGGAG | 3461 | 1604-1626 |
| AD-159737 | GGGAUCCAGUGUAUAAAUCCA | 3276 | 1657-1677 | UGGAUUUAUACACUGGAUCCCAG | 3462 | 1655-1677 |
| AD-159869 | CAAUAAACCUUGAACAGUGAA | 3277 | 1818-1838 | UUCACUGUUCAAGGUUUAUUGGG | 3463 | 1816-1838 |
| AD-158570 | GGCCUGUGCCAUCAGUAUCUU | 3278 | 371-391 | AAGAUACUGAUGGCACAGGCCAU | 3464 | 369-391 |
| AD-158618 | UUGUUGAUGUCAUCGAAGACA | 3279 | 421-441 | UGUCUUCGAUGACAUCAACAAGA | 3465 | 419-441 |
| AD-159788 | GGAUCUUAUUUUGUGAACUAU | 3280 | 1708-1728 | AUAGUUCACAAAAUAAGAUCCUU | 3466 | 1706-1728 |
| AD-159786 | AAGGAUCUUAUUUUGUGAACU | 3281 | 1706-1726 | AGUUCACAAAAUAAGAUCCUUUG | 3467 | 1704-1726 |
| AD-159760 | AUCAUGUCUUGUGCAUAAUUA | 3282 | 1680-1700 | UAAUUAUGCACAAGACAUGAUAU | 3468 | 1678-1700 |
| AD-159404 | UGUCAUAUCAUUUCACUGUCU | 3283 | 1282-1302 | AGACAGUGAAAUGAUAUGACAUC | 3469 | 1280-1302 |
| AD-159406 | UCAUAUCAUUUCACUGUCUAA | 3284 | 1284-1304 | UUAGACAGUGAAAUGAUAUGACA | 3470 | 1282-1304 |
| AD-158536 | AUUUAUAAUCUUCUAAAGGAA | 3285 | 297-317 | UUCCUUUAGAAGAUUAUAAAUCA | 3471 | 295-317 |
| AD-159545 | UGGUUUGUAAAAUCCACAGCU | 3286 | 1427-1447 | AGCUGUGGAUUUUACAAACCAUU | 3472 | 1425-1447 |
| AD-159574 | AUGCUGGAUGGUAUUAAUCUU | 3287 | 1456-1476 | AAGAUUAAUACCAUCCAGCAUCA | 3473 | 1454-1476 |
| AD-159802 | AACUAUAUCAGUAGUGUACAU | 3288 | 1723-1743 | AUGUACACUACUGAUAUAGUUCA | 3474 | 1721-1743 |
| AD-159518 | AUCAACUCCUGAAGUUAGAAA | 3289 | 1400-1420 | UUUCUAACUUCAGGAGUUGAUGU | 3475 | 1398-1420 |
| AD-159577 | CUGGAUGGUAUUAAUCUUGUA | 3290 | 1459-1479 | UACAAGAUUAAUACCAUCCAGCA | 3476 | 1457-1479 |
| AD-159409 | UAUCAUUUCACUGUCUAGGCU | 3291 | 1287-1307 | AGCCUAGACAGUGAAAUGAUAUG | 3477 | 1285-1307 |
| AD-159551 | GUAAAAUCCACAGCUAUAUCA | 3292 | 1433-1453 | UGAUAUAGCUGUGGAUUUUACAA | 3478 | 1431-1453 |
| AD-159276 | UCCUUAGUGUUCCUUGCAUUU | 3293 | 1135-1155 | AAAUGCAAGGAACACUAAGGAAG | 3479 | 1133-1155 |
| AD-159407 | CAUAUCAUUUCACUGUCUAGA | 3294 | 1285-1305 | UCUAGACAGUGAAAUGAUAUGAC | 3480 | 1283-1305 |
| AD-159515 | AACAUCAACUCCUGAAGUUAA | 3295 | 1397-1417 | UUAACUUCAGGAGUUGAUGUUUU | 3481 | 1395-1417 |
| AD-159570 | CCUGAUGCUGGAUGGUAUUAA | 3296 | 1452-1472 | UUAAUACCAUCCAGCAUCAGGAU | 3482 | 1450-1472 |
| AD-159849 | AAUGCAACCAACUAUCCAAGU | 3297 | 1777-1797 | ACUUGGAUAGUUGGUUGCAUUGU | 3483 | 1775-1797 |
| AD-159252 | UUUACGGAAUAAAGGAUGAUA | 3298 | 1111-1131 | UAUCAUCCUUUAUUCCGUAAAGA | 3484 | 1109-1131 |
| AD-159275 | UUCCUUAGUGUUCCUUGCAUU | 3299 | 1134-1154 | AAUGCAAGGAACACUAAGGAAGA | 3485 | 1132-1154 |
| AD-159848 | CAAUGCAACCAACUAUCCAAA | 3300 | 1776-1796 | UUUGGAUAGUUGGUUGCAUUGUU | 3486 | 1774-1796 |
| AD-159184 | AGAUUUGGCAGAGAGUAUAAU | 3301 | 1043-1063 | AUUAUACUCUCUGCCAAAUCUGC | 3487 | 1041-1063 |
| AD-159231 | UUUCCACCAUGAUUAAGGGUA | 3302 | 1090-1110 | UACCCUUAAUCAUGGUGGAAACU | 3488 | 1088-1110 |
| AD-159607 | ACUGGUUAGUGUGAAAUAGUU | 3303 | 1489-1509 | AACUAUUUCACACUAACCAGUUG | 3489 | 1487-1509 |
| AD-158504 | CAAGUCCAAUAUGGCAACUCU | 3304 | 263-283 | AGAGUUGCCAUAUUGGACUUGGA | 3490 | 261-283 |
| AD-159233 | UCCACCAUGAUUAAGGGUCUU | 3305 | 1092-1112 | AAGACCCUUAAUCAUGGUGGAAA | 3491 | 1090-1112 |
| AD-159411 | UCAUUUCACUGUCUAGGCUAA | 3306 | 1289-1309 | UUAGCCUAGACAGUGAAAUGAUA | 3492 | 1287-1309 |
| AD-159462 | UGUCCUUUUUAUCUGAUCUGU | 3307 | 1340-1360 | ACAGAUCAGAUAAAAAGGACAAC | 3493 | 1338-1360 |
| AD-159742 | CCAGUGUAUAAAUCCAAUAUA | 3308 | 1662-1682 | UAUAUUGGAUUUAUACACUGGAU | 3494 | 1660-1682 |
| AD-159863 | UCCAAGUGUUAUACCAACUAA | 3309 | 1791-1811 | UUAGUUGGUAUAACACUUGGAUA | 3495 | 1789-1811 |
| AD-158687 | GAACACCAAAGAUUGUCUCUA | 3310 | 490-510 | UAGAGACAAUCUUUGGUGUUCUA | 3496 | 488-510 |
| AD-158688 | AACACCAAAGAUUGUCUCUGA | 3311 | 491-511 | UCAGAGACAAUCUUUGGUGUUCU | 3497 | 489-511 |
| AD-159458 | AUGUUGUCCUUUUUAUCUGAU | 3312 | 1336-1356 | AUCAGAUAAAAAGGACAACAUGC | 3498 | 1334-1356 |
| AD-159519 | UCAACUCCUGAAGUUAGAAAU | 3313 | 1401-1421 | AUUUCUAACUUCAGGAGUUGAUG | 3499 | 1399-1421 |
| AD-159858 | AACUAUCCAAGUGUUAUACCA | 3314 | 1786-1806 | UGGUAUAACACUUGGAUAGUUGG | 3500 | 1784-1806 |
| AD-159583 | GGUAUUAAUCUUGUGUAGUCU | 3315 | 1465-1485 | AGACUACACAAGAUUAAUACCAU | 3501 | 1463-1485 |
| AD-159700 | GGCUCCUUCACUGAACAUGCA | 3316 | 1602-1622 | UGCAUGUUCAGUGAAGGAGCCAG | 3502 | 1600-1622 |
| AD-159807 | UAUCAGUAGUGUACAUUACCA | 3317 | 1728-1748 | UGGUAAUGUACACUACUGAUAUA | 3503 | 1726-1748 |
| AD-158673 | CAGCCUUUUCCUUAGAACACA | 3318 | 476-496 | UGUGUUCUAAGGAAAAGGCUGCC | 3504 | 474-496 |
| AD-159608 | CUGGUUAGUGUGAAAUAGUUA | 3319 | 1490-1510 | UAACUAUUUCACACUAACCAGUU | 3505 | 1488-1510 |
| AD-159803 | ACUAUAUCAGUAGUGUACAUU | 3320 | 1724-1744 | AAUGUACACUACUGAUAUAGUUC | 3506 | 1722-1744 |
| AD-159805 | UAUAUCAGUAGUGUACAUUAA | 3321 | 1726-1746 | UUAAUGUACACUACUGAUAUAGU | 3507 | 1724-1746 |
| AD-159489 | GUAAUAUUUUAAGAUGGACUA | 3322 | 1371-1391 | UAGUCCAUCUUAAAAUAUUACUG | 3508 | 1369-1391 |
| AD-159495 | UUUUAAGAUGGACUGGGAAAA | 3323 | 1377-1397 | UUUUCCCAGUCCAUCUUAAAAUA | 3509 | 1375-1397 |
| AD-159706 | UUCACUGAACAUGCCUAGUCA | 3324 | 1608-1628 | UGACUAGGCAUGUUCAGUGAAGG | 3510 | 1606-1628 |
| AD-159855 | ACCAACUAUCCAAGUGUUAUA | 3325 | 1783-1803 | UAUAACACUUGGAUAGUUGGUUG | 3511 | 1781-1803 |
| AD-159864 | CCAAGUGUUAUACCAACUAAA | 3326 | 1792-1812 | UUUAGUUGGUAUAACACUUGGAU | 3512 | 1790-1812 |
| AD-159488 | AGUAAUAUUUUAAGAUGGACU | 3327 | 1370-1390 | AGUCCAUCUUAAAAUAUUACUGC | 3513 | 1368-1390 |
| AD-159553 | AAAAUCCACAGCUAUAUCCUA | 3328 | 1435-1455 | UAGGAUAUAGCUGUGGAUUUUAC | 3514 | 1433-1455 |
| AD-159703 | UCCUUCACUGAACAUGCCUAA | 3329 | 1605-1625 | UUAGGCAUGUUCAGUGAAGGAGC | 3515 | 1603-1625 |
| AD-159708 | CACUGAACAUGCCUAGUCCAA | 3330 | 1610-1630 | UUGGACUAGGCAUGUUCAGUGAA | 3516 | 1608-1630 |
| AD-159866 | AAGUGUUAUACCAACUAAAAC | 3331 | 1794-1814 | GUUUUAGUUGGUAUAACACUUGG | 3517 | 1792-1814 |
| AD-159232 | UUCCACCAUGAUUAAGGGUCU | 3332 | 1091-1111 | AGACCCUUAAUCAUGGUGGAAAC | 3518 | 1089-1111 |
| AD-159712 | GAACAUGCCUAGUCCAACAUU | 3333 | 1614-1634 | AAUGUUGGACUAGGCAUGUUCAG | 3519 | 1612-1634 |
| AD-159808 | AUCAGUAGUGUACAUUACCAU | 3334 | 1729-1749 | AUGGUAAUGUACACUACUGAUAU | 3520 | 1727-1749 |
| AD-159862 | AUCCAAGUGUUAUACCAACUA | 3335 | 1790-1810 | UAGUUGGUAUAACACUUGGAUAG | 3521 | 1788-1810 |
| AD-158503 | CCAAGUCCAAUAUGGCAACUA | 3336 | 262-282 | UAGUUGCCAUAUUGGACUUGGAA | 3522 | 260-282 |
| AD-159412 | CAUUUCACUGUCUAGGCUACA | 3337 | 1290-1310 | UGUAGCCUAGACAGUGAAAUGAU | 3523 | 1288-1310 |
| AD-159558 | CCACAGCUAUAUCCUGAUGCU | 3338 | 1440-1460 | AGCAUCAGGAUAUAGCUGUGGAU | 3524 | 1438-1460 |
| AD-159705 | CUUCACUGAACAUGCCUAGUA | 3339 | 1607-1627 | UACUAGGCAUGUUCAGUGAAGGA | 3525 | 1605-1627 |
| AD-159113 | GUGGUUGAGAGUGCUUAUGAA | 3340 | 972-992 | UUCAUAAGCACUCUCAACCACCU | 3526 | 970-992 |
| AD-159806 | AUAUCAGUAGUGUACAUUACA | 3341 | 1727-1747 | UGUAAUGUACACUACUGAUAUAG | 3527 | 1725-1747 |
| AD-159853 | CAACCAACUAUCCAAGUGUUA | 3342 | 1781-1801 | UAACACUUGGAUAGUUGGUUGCA | 3528 | 1779-1801 |
| AD-159182 | GCAGAUUUGGCAGAGAGUAUA | 3343 | 1041-1061 | UAUACUCUCUGCCAAAUCUGCUA | 3529 | 1039-1061 |
| AD-159702 | CUCCUUCACUGAACAUGCCUA | 3344 | 1604-1624 | UAGGCAUGUUCAGUGAAGGAGCC | 3530 | 1602-1624 |
| AD-159715 | CAUGCCUAGUCCAACAUUUUU | 3345 | 1617-1637 | AAAAAUGUUGGACUAGGCAUGUU | 3531 | 1615-1637 |
| AD-158575 | UGCCAUCAGUAUCUUAAUGAA | 3346 | 377-397 | UUCAUUAAGAUACUGAUGGCACA | 3532 | 375-397 |
| AD-158576 | GCCAUCAGUAUCUUAAUGAAA | 3347 | 378-398 | UUUCAUUAAGAUACUGAUGGCAC | 3533 | 376-398 |
| AD-158684 | UUAGAACACCAAAGAUUGUCU | 3348 | 487-507 | AGACAAUCUUUGGUGUUCUAAGG | 3534 | 485-507 |
| AD-159410 | AUCAUUUCACUGUCUAGGCUA | 3349 | 1288-1308 | UAGCCUAGACAGUGAAAUGAUAU | 3535 | 1286-1308 |
| AD-159416 | UCACUGUCUAGGCUACAACAA | 3350 | 1294-1314 | UUGUUGUAGCCUAGACAGUGAAA | 3536 | 1292-1314 |
| AD-159857 | CAACUAUCCAAGUGUUAUACA | 3351 | 1785-1805 | UGUAUAACACUUGGAUAGUUGGU | 3537 | 1783-1805 |
| AD-158497 | UUGGUUCCAAGUCCAAUAUGA | 3352 | 256-276 | UCAUAUUGGACUUGGAACCAAAA | 3538 | 254-276 |
| AD-159124 | UGCUUAUGAGGUGAUCAAACU | 3353 | 983-1003 | AGUUUGAUCACCUCAUAAGCACU | 3539 | 981-1003 |
| AD-159312 | UCUCAGACCUUGUGAAGGUGA | 3354 | 1171-1191 | UCACCUUCACAAGGUCUGAGAUU | 3540 | 1169-1191 |
| AD-159552 | UAAAAUCCACAGCUAUAUCCU | 3355 | 1434-1454 | AGGAUAUAGCUGUGGAUUUUACA | 3541 | 1432-1454 |
| AD-159704 | CCUUCACUGAACAUGCCUAGU | 3356 | 1606-1626 | ACUAGGCAUGUUCAGUGAAGGAG | 3542 | 1604-1626 |
| AD-159737 | GGGAUCCAGUGUAUAAAUCCA | 3357 | 1657-1677 | UGGAUUUAUACACUGGAUCCCAG | 3543 | 1655-1677 |
| AD-159869 | CAAUAAACCUUGAACAGUGAA | 3358 | 1818-1838 | UUCACUGUUCAAGGUUUAUUGGG | 3544 | 1816-1838 |
| AD-158570 | GGCCUGUGCCAUCAGUAUCUU | 3359 | 371-391 | AAGAUACUGAUGGCACAGGCCAU | 3545 | 369-391 |
| AD-158618 | UUGUUGAUGUCAUCGAAGACA | 3360 | 421-441 | UGUCUUCGAUGACAUCAACAAGA | 3546 | 419-441 |
| AD-159184 | AGAUUUGGCAGAGAGUAUAAU | 3361 | 1043-1063 | AUUAUACUCUCUGCCAAAUCUGC | 3547 | 1041-1063 |
| AD-159231 | UUUCCACCAUGAUUAAGGGUA | 3362 | 1090-1110 | UACCCUUAAUCAUGGUGGAAACU | 3548 | 1088-1110 |
| AD-159423 | CUAGGCUACAACAGGAUUCUA | 3363 | 1301-1321 | UAGAAUCCUGUUGUAGCCUAGAC | 3549 | 1299-1321 |
| AD-159446 | UGGAGGUUGUGCAUGUUGUCA | 3364 | 1324-1344 | UGACAACAUGCACAACCUCCACC | 3550 | 1322-1344 |
| AD-159701 | GCUCCUUCACUGAACAUGCCU | 3365 | 1603-1623 | AGGCAUGUUCAGUGAAGGAGCCA | 3551 | 1601-1623 |
| AD-158494 | CUUUUGGUUCCAAGUCCAAUA | 3366 | 253-273 | UAUUGGACUUGGAACCAAAAGGA | 3552 | 251-273 |
| AD-158571 | GCCUGUGCCAUCAGUAUCUUA | 3367 | 372-392 | UAAGAUACUGAUGGCACAGGCCA | 3553 | 370-392 |
| AD-159125 | GCUUAUGAGGUGAUCAAACUA | 3368 | 984-1004 | UAGUUUGAUCACCUCAUAAGCAC | 3554 | 982-1004 |
| AD-159126 | CUUAUGAGGUGAUCAAACUCA | 3369 | 985-1005 | UGAGUUUGAUCACCUCAUAAGCA | 3555 | 983-1005 |
| AD-159287 | CCUUGCAUUUUGGGACAGAAU | 3370 | 1146-1166 | AUUCUGUCCCAAAAUGCAAGGAA | 3556 | 1144-1166 |
| AD-158499 | GGUUCCAAGUCCAAUAUGGCA | 3371 | 258-278 | UGCCAUAUUGGACUUGGAACCAA | 3557 | 256-278 |
| AD-159417 | CACUGUCUAGGCUACAACAGA | 3372 | 1295-1315 | UCUGUUGUAGCCUAGACAGUGAA | 3558 | 1293-1315 |
| AD-159418 | ACUGUCUAGGCUACAACAGGA | 3373 | 1296-1316 | UCCUGUUGUAGCCUAGACAGUGA | 3559 | 1294-1316 |
| AD-158550 | AAUAAGAUUACAGUUGUUGGA | 3374 | 333-353 | UCCAACAACUGUAAUCUUAUUCU | 3560 | 331-353 |
| AD-159116 | GUUGAGAGUGCUUAUGAGGUA | 3375 | 975-995 | UACCUCAUAAGCACUCUCAACCA | 3561 | 973-995 |
| AD-159421 | GUCUAGGCUACAACAGGAUUA | 3376 | 1299-1319 | UAAUCCUGUUGUAGCCUAGACAG | 3562 | 1297-1319 |
| AD-159422 | UCUAGGCUACAACAGGAUUCU | 3377 | 1300-1320 | AGAAUCCUGUUGUAGCCUAGACA | 3563 | 1298-1320 |
| AD-159445 | GUGGAGGUUGUGCAUGUUGUA | 3378 | 1323-1343 | UACAACAUGCACAACCUCCACCU | 3564 | 1321-1343 |
| AD-159130 | UGAGGUGAUCAAACUCAAAGA | 3379 | 989-1009 | UCUUUGAGUUUGAUCACCUCAUA | 3565 | 987-1009 |
| AD-159134 | GUGAUCAAACUCAAAGGCUAA | 3380 | 993-1013 | UUAGCCUUUGAGUUUGAUCACCU | 3566 | 991-1013 |
| AD-159343 | UGAGGAAGAGGCCCGUUUGAA | 3381 | 1202-1222 | UUCAAACGGGCCUCUUCCUCAGA | 3567 | 1200-1222 |
| AD-159105 | ACAAGCAGGUGGUUGAGAGUA | 3382 | 964-984 | UACUCUCAACCACCUGCUUGUGA | 3568 | 962-984 |
| AD-159183 | CAGAUUUGGCAGAGAGUAUAA | 3383 | 1042-1062 | UUAUACUCUCUGCCAAAUCUGCU | 3569 | 1040-1062 |
| AD-159123 | GUGCUUAUGAGGUGAUCAAAC | 3384 | 982-1002 | GUUUGAUCACCUCAUAAGCACUC | 3570 | 980-1002 |
| AD-159181 | AGCAGAUUUGGCAGAGAGUAU | 3385 | 1040-1060 | AUACUCUCUGCCAAAUCUGCUAC | 3571 | 1038-1060 |
| AD-159186 | AUUUGGCAGAGAGUAUAAUGA | 3386 | 1045-1065 | UCAUUAUACUCUCUGCCAAAUCU | 3572 | 1043-1065 |
| AD-159187 | UUUGGCAGAGAGUAUAAUGAA | 3387 | 1046-1066 | UUCAUUAUACUCUCUGCCAAAUC | 3573 | 1044-1066 |
| AD-159288 | CUUGCAUUUUGGGACAGAAUA | 3388 | 1147-1167 | UAUUCUGUCCCAAAAUGCAAGGA | 3574 | 1145-1167 |
| AD-159306 | AUGGAAUCUCAGACCUUGUGA | 3389 | 1165-1185 | UCACAAGGUCUGAGAUUCCAUUC | 3575 | 1163-1185 |
| AD-159559 | CACAGCUAUAUCCUGAUGCUA | 3390 | 1441-1461 | UAGCAUCAGGAUAUAGCUGUGGA | 3576 | 1439-1461 |
| AD-159344 | GAGGAAGAGGCCCGUUUGAAA | 3391 | 1203-1223 | UUUCAAACGGGCCUCUUCCUCAG | 3577 | 1201-1223 |
| AD-159341 | UCUGAGGAAGAGGCCCGUUUA | 3392 | 1200-1220 | UAAACGGGCCUCUUCCUCAGAAG | 3578 | 1198-1220 |
| AD-159729 | CACAUCCUGGGAUCCAGUGUA | 3393 | 1649-1669 | UACACUGGAUCCCAGGAUGUGAC | 3579 | 1647-1669 |
| AD-158674 | AGCCUUUUCCUUAGAACACCA | 3394 | 477-497 | UGGUGUUCUAAGGAAAAGGCUGC | 3580 | 475-497 |
| AD-159604 | UCAACUGGUUAGUGUGAAAUA | 3395 | 1486-1506 | UAUUUCACACUAACCAGUUGAAG | 3581 | 1484-1506 |

TABLE 3
MODIFIED HUMAN/CYNOMOLGUS CROSS-REACTIVE LDHA IRNA SEQUENCES

| Duplex | | SEQ ID | | SEQ ID | | SEQ ID |
|---|---|---|---|---|---|---|
| Name | Sense Sequence 5′ to 3′ | NO | Antisense Sequence 5′ to 3′ | NO | mRNA target sequence | NO |
| AD-159469 | ususuaucUfgAfUfCfugugauuaaaL96 | 3582 | usUfsuaaUfcAfCfagauCfaGfauaaasasa | 3768 | UUUUUAUCUGAUCUGUGAUUAAA | 3954 |
| AD-159607 | ascsugguUfaGfUfGfugaaauaguuL96 | 3583 | asAfscuaUfuUfCfacacUfaAfccagususg | 3769 | CAACUGGUUAGUGUGAAAUAGUU | 3955 |
| AD-159713 | asascaugCfcUfAfGfuccaacauuuL96 | 3584 | asAfsaugUfuGfGfacuaGfgCfauguuscsa | 3770 | UGAACAUGCCUAGUCCAACAUUU | 3956 |
| AD-158504 | csasagucCfaAfUfAfuggcaacucuL96 | 3585 | asGfsaguUfgCfCfauauUfgGfacuugsgsa | 3771 | UCCAAGUCCAAUAUGGCAACUCU | 3957 |
| AD-159233 | uscscaccAfuGfAfUfuaagggucuuL96 | 3586 | asAfsgacCfcUfUfaaucAfuGfguggasasa | 3772 | UUUCCACCAUGAUUAAGGGUCUU | 3958 |
| AD-159411 | uscsauuuCfaCfUfGfucuaggcuaaL96 | 3587 | usUfsagcCfuAfGfacagUfgAfaaugasusa | 3773 | UAUCAUUUCACUGUCUAGGCUAC | 3959 |
| AD-159462 | usgsuccuUfuUfUfAfucugaucuguL96 | 3588 | asCfsagaUfcAfGfauaaAfaAfggacasasc | 3774 | GUUGUCCUUUUUAUCUGAUCUGU | 3960 |
| AD-159742 | cscsagugUfaUfAfAfauccaauauaL96 | 3589 | usAfsuauUfgGfAfuuuaUfaCfacuggsasu | 3775 | AUCCAGUGUAUAAAUCCAAUAUC | 3961 |
| AD-159863 | uscscaagUfgUfUfAfuaccaacuaaL96 | 3590 | usUfsaguUfgGfUfauaaCfaCfuuggasusa | 3776 | UAUCCAAGUGUUAUACCAACUAA | 3962 |
| AD-158626 | gsuscaucGfaAfGfAfcaaauugaaaL96 | 3591 | usUfsucaAfuUfUfgucuUfcGfaugacsasu | 3777 | AUGUCAUCGAAGACAAAUUGAAG | 3963 |
| AD-158687 | gsasacacCfaAfAfGfauugucucuaL96 | 3592 | usAfsgagAfcAfAfucuuUfgGfuguucsusa | 3778 | UAGAACACCAAAGAUUGUCUCUG | 3964 |
| AD-158688 | asascaccAfaAfGfAfuugucucugaL96 | 3593 | usCfsagaGfaCfAfaucuUfuGfguguuscsu | 3779 | AGAACACCAAAGAUUGUCUCUGG | 3965 |
| AD-159458 | asusguugUfcCfUfUfuuuaucugauL96 | 3594 | asUfscagAfuAfAfaaagGfaCfaacausgsc | 3780 | GCAUGUUGUCCUUUUUAUCUGAU | 3966 |
| AD-159519 | uscsaacuCfcUfGfAfaguuagaaauL96 | 3595 | asUfsuucUfaAfCfuucaGfgAfguugasusg | 3781 | CAUCAACUCCUGAAGUUAGAAAU | 3967 |
| AD-159858 | asascuauCfcAfAfGfuguuauaccaL96 | 3596 | usGfsguaUfaAfCfacuuGfgAfuaguusgsg | 3782 | CCAACUAUCCAAGUGUUAUACCA | 3968 |
| AD-158681 | uscscuuaGfaAfCfAfccaaagauuaL96 | 3597 | usAfsaucUfuUfGfguguUfcUfaaggasasa | 3783 | UUUCCUUAGAACACCAAAGAUUG | 3969 |
| AD-159583 | gsgsuauuAfaUfCfUfuguguagucuL96 | 3598 | asGfsacuAfcAfCfaagaUfuAfauaccsasu | 3784 | AUGGUAUUAAUCUUGUGUAGUCU | 3970 |
| AD-159700 | gsgscuccUfuCfAfCfugaacaugcaL96 | 3599 | usGfscauGfuUfCfagugAfaGfgagccsasg | 3785 | CUGGCUCCUUCACUGAACAUGCC | 3971 |
| AD-159807 | usasucagUfaGfUfGfuacauuaccaL96 | 3600 | usGfsguaAfuGfUfacacUfaCfugauasusa | 3786 | UAUAUCAGUAGUGUACAUUACCA | 3972 |
| AD-158673 | csasgccuUfuUfCfCfuuagaacacaL96 | 3601 | usGfsuguUfcUfAfaggaAfaAfggcugscsc | 3787 | GGCAGCCUUUUCCUUAGAACACC | 3973 |
| AD-159608 | csusgguuAfgUfGfUfgaaauaguuaL96 | 3602 | usAfsacuAfuUfUfcacaCfuAfaccagsusu | 3788 | AACUGGUUAGUGUGAAAUAGUUC | 3974 |
| AD-159803 | ascsuauaUfcAfGfUfaguguacauuL96 | 3603 | asAfsuguAfcAfCfuacuGfaUfauagususc | 3789 | GAACUAUAUCAGUAGUGUACAUU | 3975 |
| AD-159805 | usasuaucAfgUfAfGfuguacauuaaL96 | 3604 | usUfsaauGfuAfCfacuaCfuGfauauasgsu | 3790 | ACUAUAUCAGUAGUGUACAUUAC | 3976 |
| AD-159489 | gsusaauaUfuUfUfAfagauggacuaL96 | 3605 | usAfsgucCfaUfCfuuaaAfaUfauuacsusg | 3791 | CAGUAAUAUUUUAAGAUGGACUG | 3977 |
| AD-159495 | ususuuaaGfaUfGfGfacugggaaaaL96 | 3606 | usUfsuucCfcAfGfuccaUfcUfuaaaasusa | 3792 | UAUUUUAAGAUGGACUGGGAAAA | 3978 |
| AD-159609 | usgsguuaGfuGfUfGfaaauaguucuL96 | 3607 | asGfsaacUfaUfUfucacAfcUfaaccasgsu | 3793 | ACUGGUUAGUGUGAAAUAGUUCU | 3979 |
| AD-159706 | ususcacuGfaAfCfAfugccuagucaL96 | 3608 | usGfsacuAfgGfCfauguUfcAfgugaasgsg | 3794 | CCUUCACUGAACAUGCCUAGUCC | 3980 |
| AD-159855 | ascscaacUfaUfCfCfaaguguuauaL96 | 3609 | usAfsuaaCfaCfUfuggaUfaGfuuggususg | 3795 | CAACCAACUAUCCAAGUGUUAUA | 3981 |
| AD-159864 | cscsaaguGfuUfAfUfaccaacuaaaL96 | 3610 | usUfsuagUfuGfGfuauaAfcAfcuuggsasu | 3796 | AUCCAAGUGUUAUACCAACUAAA | 3982 |
| AD-158491 | ususccuuUfuGfGfUfuccaaguccaL96 | 3611 | usGfsgacUfuGfGfaaccAfaAfaggaasusc | 3797 | GAUUCCUUUUGGUUCCAAGUCCA | 3983 |
| AD-158672 | gscsagccUfuUfUfCfcuuagaacaaL96 | 3612 | usUfsguuCfuAfAfggaaAfaGfgcugcscsa | 3798 | UGGCAGCCUUUUCCUUAGAACAC | 3984 |
| AD-159488 | asgsuaauAfuUfUfUfaagauggacuL96 | 3613 | asGfsuccAfuCfUfuaaaAfuAfuuacusgsc | 3799 | GCAGUAAUAUUUUAAGAUGGACU | 3985 |
| AD-159553 | asasaaucCfaCfAfGfcuauauccuaL96 | 3614 | usAfsggaUfaUfAfgcugUfgGfauuuusasc | 3800 | GUAAAAUCCACAGCUAUAUCCUG | 3986 |
| AD-159703 | uscscuucAfcUfGfAfacaugccuaaL96 | 3615 | usUfsaggCfaUfGfuucaGfuGfaaggasgsc | 3801 | GCUCCUUCACUGAACAUGCCUAG | 3987 |
| AD-159708 | csascugaAfcAfUfGfccuaguccaaL96 | 3616 | usUfsggaCfuAfGfgcauGfuUfcagugsasa | 3802 | UUCACUGAACAUGCCUAGUCCAA | 3988 |
| AD-159866 | asasguguUfaUfAfCfcaacuaaaacL96 | 3617 | gsUfsuuuAfgUfUfgguaUfaAfcacuusgsg | 3803 | CCAAGUGUUAUACCAACUAAAAC | 3989 |
| AD-159232 | ususccacCfaUfGfAfuuaagggucuL96 | 3618 | asGfsaccCfuUfAfaucaUfgGfuggaasasc | 3804 | GUUUCCACCAUGAUUAAGGGUCU | 3990 |
| AD-159712 | gsasacauGfcCfUfAfguccaacauuL96 | 3619 | asAfsuguUfgGfAfcuagGfcAfuguucsasg | 3805 | CUGAACAUGCCUAGUCCAACAUU | 3991 |
| AD-159808 | asuscaguAfgUfGfUfacauuaccauL96 | 3620 | asUfsgguAfaUfGfuacaCfuAfcugausasu | 3806 | AUAUCAGUAGUGUACAUUACCAU | 3992 |
| AD-159862 | asusccaaGfuGfUfUfauaccaacuaL96 | 3621 | usAfsguuGfgUfAfuaacAfcUfuggausasg | 3807 | CUAUCCAAGUGUUAUACCAACUA | 3993 |
| AD-158503 | cscsaaguCfcAfAfUfauggcaacuaL96 | 3622 | usAfsguuGfcCfAfuauuGfgAfcuuggsasa | 3808 | UUCCAAGUCCAAUAUGGCAACUC | 3994 |
| AD-159311 | asuscucaGfaCfCfUfugugaagguaL96 | 3623 | usAfsccuUfcAfCfaaggUfcUfgagaususc | 3809 | GAAUCUCAGACCUUGUGAAGGUG | 3995 |
| AD-159412 | csasuuucAfcUfGfUfcuaggcuacaL96 | 3624 | usGfsuagCfcUfAfgacaGfuGfaaaugsasu | 3810 | AUCAUUUCACUGUCUAGGCUACA | 3996 |
| AD-159558 | cscsacagCfuAfUfAfuccugaugcuL96 | 3625 | asGfscauCfaGfGfauauAfgCfuguggsasu | 3811 | AUCCACAGCUAUAUCCUGAUGCU | 3997 |
| AD-159705 | csusucacUfgAfAfCfaugccuaguaL96 | 3626 | usAfscuaGfgCfAfuguuCfaGfugaagsgsa | 3812 | UCCUUCACUGAACAUGCCUAGUC | 3998 |
| AD-159113 | gsusgguuGfaGfAfGfugcuuaugaaL96 | 3627 | usUfscauAfaGfCfacucUfcAfaccacscsu | 3813 | AGGUGGUUGAGAGUGCUUAUGAG | 3999 |
| AD-159139 | csasaacuCfaAfAfGfgcuacacauaL96 | 3628 | usAfsuguGfuAfGfccuuUfgAfguuugsasu | 3814 | AUCAAACUCAAAGGCUACACAUC | 4000 |
| AD-159806 | asusaucaGfuAfGfUfguacauuacaL96 | 3629 | usGfsuaaUfgUfAfcacuAfcUfgauausasg | 3815 | CUAUAUCAGUAGUGUACAUUACC | 4001 |
| AD-159853 | csasaccaAfcUfAfUfccaaguguuaL96 | 3630 | usAfsacaCfuUfGfgauaGfuUfgguugscsa | 3816 | UGCAACCAACUAUCCAAGUGUUA | 4002 |
| AD-158627 | uscsaucgAfaGfAfCfaaauugaagaL96 | 3631 | usCfsuucAfaUfUfugucUfuCfgaugascsa | 3817 | UGUCAUCGAAGACAAAUUGAAGG | 4003 |
| AD-159182 | gscsagauUfuGfGfCfagagaguauaL96 | 3632 | usAfsuacUfcUfCfugccAfaAfucugcsusa | 3818 | UAGCAGAUUUGGCAGAGAGUAUA | 4004 |
| AD-159702 | csusccuuCfaCfUfGfaacaugccuaL96 | 3633 | usAfsggcAfuGfUfucagUfgAfaggagscsc | 3819 | GGCUCCUUCACUGAACAUGCCUA | 4005 |
| AD-159715 | csasugccUfaGfUfCfcaacauuuuuL96 | 3634 | asAfsaaaUfgUfUfggacUfaGfgcaugsusu | 3820 | AACAUGCCUAGUCCAACAUUUUU | 4006 |
| AD-158575 | usgsccauCfaGfUfAfucuuaaugaaL96 | 3635 | usUfscauUfaAfGfauacUfgAfuggcascsa | 3821 | UGUGCCAUCAGUAUCUUAAUGAA | 4007 |
| AD-158576 | gscscaucAfgUfAfUfcuuaaugaaaL96 | 3636 | usUfsucaUfuAfAfgauaCfuGfauggcsasc | 3822 | GUGCCAUCAGUAUCUUAAUGAAG | 4008 |
| AD-158684 | ususagaaCfaCfCfAfaagauugucuL96 | 3637 | asGfsacaAfuCfUfuuggUfgUfucuaasgsg | 3823 | CCUUAGAACACCAAAGAUUGUCU | 4009 |
| AD-159410 | asuscauuUfcAfCfUfgucuaggcuaL96 | 3638 | usAfsgccUfaGfAfcaguGfaAfaugausasu | 3824 | AUAUCAUUUCACUGUCUAGGCUA | 4010 |
| AD-159416 | uscsacugUfcUfAfGfgcuacaacaaL96 | 3639 | usUfsguuGfuAfGfccuaGfaCfagugasasa | 3825 | UUUCACUGUCUAGGCUACAACAG | 4011 |
| AD-159738 | gsgsauccAfgUfGfUfauaaauccaaL96 | 3640 | usUfsggaUfuUfAfuacaCfuGfgauccscsa | 3826 | UGGGAUCCAGUGUAUAAAUCCAA | 4012 |
| AD-159857 | csasacuaUfcCfAfAfguguuauacaL96 | 3641 | usGfsuauAfaCfAfcuugGfaUfaguugsgsu | 3827 | ACCAACUAUCCAAGUGUUAUACC | 4013 |
| AD-158497 | ususgguuCfcAfAfGfuccaauaugaL96 | 3642 | usCfsauaUfuGfGfacuuGfgAfaccaasasa | 3828 | UUUUGGUUCCAAGUCCAAUAUGG | 4014 |
| AD-159124 | usgscuuaUfgAfGfGfugaucaaacuL96 | 3643 | asGfsuuuGfaUfCfaccuCfaUfaagcascsu | 3829 | AGUGCUUAUGAGGUGAUCAAACU | 4015 |
| AD-159140 | asasacucAfaAfGfGfcuacacaucaL96 | 3644 | usGfsaugUfgUfAfgccuUfuGfaguuusgsa | 3830 | UCAAACUCAAAGGCUACACAUCC | 4016 |
| AD-159312 | uscsucagAfcCfUfUfgugaaggugaL96 | 3645 | usCfsaccUfuCfAfcaagGfuCfugagasusu | 3831 | AAUCUCAGACCUUGUGAAGGUGA | 4017 |
| AD-159552 | usasaaauCfcAfCfAfgcuauauccuL96 | 3646 | asGfsgauAfuAfGfcuguGfgAfuuuuascsa | 3832 | UGUAAAAUCCACAGCUAUAUCCU | 4018 |
| AD-159704 | cscsuucaCfuGfAfAfcaugccuaguL96 | 3647 | asCfsuagGfcAfUfguucAfgUfgaaggsasg | 3833 | CUCCUUCACUGAACAUGCCUAGU | 4019 |
| AD-159737 | gsgsgaucCfaGfUfGfuauaaauccaL96 | 3648 | usGfsgauUfuAfUfacacUfgGfaucccsasg | 3834 | CUGGGAUCCAGUGUAUAAAUCCA | 4020 |
| AD-159869 | csasauaaAfcCfUfUfgaacagugaaL96 | 3649 | usUfscacUfgUfUfcaagGfuUfuauugsgsg | 3835 | CCCAAUAAACCUUGAACAGUGAC | 4021 |
| AD-158570 | gsgsccugUfgCfCfAfucaguaucuuL96 | 3650 | asAfsgauAfcUfGfauggCfaCfaggccsasu | 3836 | AUGGCCUGUGCCAUCAGUAUCUU | 4022 |
| AD-158618 | ususguugAfuGfUfCfaucgaagacaL96 | 3651 | usGfsucuUfcGfAfugacAfuCfaacaasgsa | 3837 | UCUUGUUGAUGUCAUCGAAGACA | 4023 |
| AD-159788 | gsgsaucuUfaUfUfUfugugaacuauL96 | 3652 | asUfsaguUfcAfCfaaaaUfaAfgauccsusu | 3838 | AAGGAUCUUAUUUUGUGAACUAU | 4024 |
| AD-159786 | asasggauCfuUfAfUfuuugugaacuL96 | 3653 | asGfsuucAfcAfAfaauaAfgAfuccuususg | 3839 | CAAAGGAUCUUAUUUUGUGAACU | 4025 |
| AD-159760 | asuscaugUfcUfUfGfugcauaauuaL96 | 3654 | usAfsauuAfuGfCfacaaGfaCfaugausasu | 3840 | AUAUCAUGUCUUGUGCAUAAUUC | 4026 |
| AD-159404 | usgsucauAfuCfAfUfuucacugucuL96 | 3655 | asGfsacaGfuGfAfaaugAfuAfugacasusc | 3841 | GAUGUCAUAUCAUUUCACUGUCU | 4027 |
| AD-159406 | uscsauauCfaUfUfUfcacugucuaaL96 | 3656 | usUfsagaCfaGfUfgaaaUfgAfuaugascsa | 3842 | UGUCAUAUCAUUUCACUGUCUAG | 4028 |
| AD-158536 | asusuuauAfaUfCfUfucuaaaggaaL96 | 3657 | usUfsccuUfuAfGfaagaUfuAfuaaauscsa | 3843 | UGAUUUAUAAUCUUCUAAAGGAA | 4029 |
| AD-159545 | usgsguuuGfuAfAfAfauccacagcuL96 | 3658 | asGfscugUfgGfAfuuuuAfcAfaaccasusu | 3844 | AAUGGUUUGUAAAAUCCACAGCU | 4030 |
| AD-159574 | asusgcugGfaUfGfGfuauuaaucuuL96 | 3659 | asAfsgauUfaAfUfaccaUfcCfagcauscsa | 3845 | UGAUGCUGGAUGGUAUUAAUCUU | 4031 |
| AD-159802 | asascuauAfuCfAfGfuaguguacauL96 | 3660 | asUfsguaCfaCfUfacugAfuAfuaguuscsa | 3846 | UGAACUAUAUCAGUAGUGUACAU | 4032 |
| AD-159518 | asuscaacUfcCfUfGfaaguuagaaaL96 | 3661 | usUfsucuAfaCfUfucagGfaGfuugausgsu | 3847 | ACAUCAACUCCUGAAGUUAGAAA | 4033 |
| AD-159577 | csusggauGfgUfAfUfuaaucuuguaL96 | 3662 | usAfscaaGfaUfUfaauaCfcAfuccagscsa | 3848 | UGCUGGAUGGUAUUAAUCUUGUG | 4034 |
| AD-159409 | usasucauUfuCfAfCfugucuaggcuL96 | 3663 | asGfsccuAfgAfCfagugAfaAfugauasusg | 3849 | CAUAUCAUUUCACUGUCUAGGCU | 4035 |
| AD-159551 | gsusaaaaUfcCfAfCfagcuauaucaL96 | 3664 | usGfsauaUfaGfCfugugGfaUfuuuacsasa | 3850 | UUGUAAAAUCCACAGCUAUAUCC | 4036 |
| AD-159276 | uscscuuaGfuGfUfUfccuugcauuuL96 | 3665 | asAfsaugCfaAfGfgaacAfcUfaaggasasg | 3851 | CUUCCUUAGUGUUCCUUGCAUUU | 4037 |
| AD-159407 | csasuaucAfuUfUfCfacugucuagaL96 | 3666 | usCfsuagAfcAfGfugaaAfuGfauaugsasc | 3852 | GUCAUAUCAUUUCACUGUCUAGG | 4038 |
| AD-159515 | asascaucAfaCfUfCfcugaaguuaaL96 | 3667 | usUfsaacUfuCfAfggagUfuGfauguususu | 3853 | AAAACAUCAACUCCUGAAGUUAG | 4039 |
| AD-159570 | cscsugauGfcUfGfGfaugguauuaaL96 | 3668 | usUfsaauAfcCfAfuccaGfcAfucaggsasu | 3854 | AUCCUGAUGCUGGAUGGUAUUAA | 4040 |
| AD-159849 | asasugcaAfcCfAfAfcuauccaaguL96 | 3669 | asCfsuugGfaUfAfguugGfuUfgcauusgsu | 3855 | ACAAUGCAACCAACUAUCCAAGU | 4041 |
| AD-159252 | ususuacgGfaAfUfAfaaggaugauaL96 | 3670 | usAfsucaUfcCfUfuuauUfcCfguaaasgsa | 3856 | UCUUUACGGAAUAAAGGAUGAUG | 4042 |
| AD-159275 | ususccuuAfgUfGfUfuccuugcauuL96 | 3671 | asAfsugcAfaGfGfaacaCfuAfaggaasgsa | 3857 | UCUUCCUUAGUGUUCCUUGCAUU | 4043 |
| AD-159848 | csasaugcAfaCfCfAfacuauccaaaL96 | 3672 | usUfsuggAfuAfGfuuggUfuGfcauugsusu | 3858 | AACAAUGCAACCAACUAUCCAAG | 4044 |
| AD-159184 | asgsauuuGfgCfAfGfagaguauaauL96 | 3673 | asUfsuauAfcUfCfucugCfcAfaaucusgsc | 3859 | GCAGAUUUGGCAGAGAGUAUAAU | 4045 |
| AD-159231 | ususuccaCfcAfUfGfauuaaggguaL96 | 3674 | usAfscccUfuAfAfucauGfgUfggaaascsu | 3860 | AGUUUCCACCAUGAUUAAGGGUC | 4046 |
| AD-159607 | ascsugguUfaGfUfGfugaaauaguuL96 | 3675 | asAfscuaUfuUfCfacacUfaAfccagususg | 3861 | CAACUGGUUAGUGUGAAAUAGUU | 4047 |
| AD-158504 | csasagucCfaAfUfAfuggcaacucuL96 | 3676 | asGfsaguUfgCfCfauauUfgGfacuugsgsa | 3862 | UCCAAGUCCAAUAUGGCAACUCU | 4048 |
| AD-159233 | uscscaccAfuGfAfUfuaagggucuuL96 | 3677 | asAfsgacCfcUfUfaaucAfuGfguggasasa | 3863 | UUUCCACCAUGAUUAAGGGUCUU | 4049 |
| AD-159411 | uscsauuuCfaCfUfGfucuaggcuaaL96 | 3678 | usUfsagcCfuAfGfacagUfgAfaaugasusa | 3864 | UAUCAUUUCACUGUCUAGGCUAC | 4050 |
| AD-159462 | usgsuccuUfuUfUfAfucugaucuguL96 | 3679 | asCfsagaUfcAfGfauaaAfaAfggacasasc | 3865 | GUUGUCCUUUUUAUCUGAUCUGU | 4051 |
| AD-159742 | cscsagugUfaUfAfAfauccaauauaL96 | 3680 | usAfsuauUfgGfAfuuuaUfaCfacuggsasu | 3866 | AUCCAGUGUAUAAAUCCAAUAUC | 4052 |
| AD-159863 | uscscaagUfgUfUfAfuaccaacuaaL96 | 3681 | usUfsaguUfgGfUfauaaCfaCfuuggasusa | 3867 | UAUCCAAGUGUUAUACCAACUAA | 4053 |
| AD-158687 | gsasacacCfaAfAfGfauugucucuaL96 | 3682 | usAfsgagAfcAfAfucuuUfgGfuguucsusa | 3868 | UAGAACACCAAAGAUUGUCUCUG | 4054 |
| AD-158688 | asascaccAfaAfGfAfuugucucugaL96 | 3683 | usCfsagaGfaCfAfaucuUfuGfguguuscsu | 3869 | AGAACACCAAAGAUUGUCUCUGG | 4055 |
| AD-159458 | asusguugUfcCfUfUfuuuaucugauL96 | 3684 | asUfscagAfuAfAfaaagGfaCfaacausgsc | 3870 | GCAUGUUGUCCUUUUUAUCUGAU | 4056 |
| AD-159519 | uscsaacuCfcUfGfAfaguuagaaauL96 | 3685 | asUfsuucUfaAfCfuucaGfgAfguugasusg | 3871 | CAUCAACUCCUGAAGUUAGAAAU | 4057 |
| AD-159858 | asascuauCfcAfAfGfuguuauaccaL96 | 3686 | usGfsguaUfaAfCfacuuGfgAfuaguusgsg | 3872 | CCAACUAUCCAAGUGUUAUACCA | 4058 |
| AD-159583 | gsgsuauuAfaUfCfUfuguguagucuL96 | 3687 | asGfsacuAfcAfCfaagaUfuAfauaccsasu | 3873 | AUGGUAUUAAUCUUGUGUAGUCU | 4059 |
| AD-159700 | gsgscuccUfuCfAfCfugaacaugcaL96 | 3688 | usGfscauGfuUfCfagugAfaGfgagccsasg | 3874 | CUGGCUCCUUCACUGAACAUGCC | 4060 |
| AD-159807 | usasucagUfaGfUfGfuacauuaccaL96 | 3689 | usGfsguaAfuGfUfacacUfaCfugauasusa | 3875 | UAUAUCAGUAGUGUACAUUACCA | 4061 |
| AD-158673 | csasgccuUfuUfCfCfuuagaacacaL96 | 3690 | usGfsuguUfcUfAfaggaAfaAfggcugscsc | 3876 | GGCAGCCUUUUCCUUAGAACACC | 4062 |
| AD-159608 | csusgguuAfgUfGfUfgaaauaguuaL96 | 3691 | usAfsacuAfuUfUfcacaCfuAfaccagsusu | 3877 | AACUGGUUAGUGUGAAAUAGUUC | 4063 |
| AD-159803 | ascsuauaUfcAfGfUfaguguacauuL96 | 3692 | asAfsuguAfcAfCfuacuGfaUfauagususc | 3878 | GAACUAUAUCAGUAGUGUACAUU | 4064 |
| AD-159805 | usasuaucAfgUfAfGfuguacauuaaL96 | 3693 | usUfsaauGfuAfCfacuaCfuGfauauasgsu | 3879 | ACUAUAUCAGUAGUGUACAUUAC | 4065 |
| AD-159489 | gsusaauaUfuUfUfAfagauggacuaL96 | 3694 | usAfsgucCfaUfCfuuaaAfaUfauuacsusg | 3880 | CAGUAAUAUUUUAAGAUGGACUG | 4066 |
| AD-159495 | ususuuaaGfaUfGfGfacugggaaaaL96 | 3695 | usUfsuucCfcAfGfuccaUfcUfuaaaasusa | 3881 | UAUUUUAAGAUGGACUGGGAAAA | 4067 |
| AD-159706 | ususcacuGfaAfCfAfugccuagucaL96 | 3696 | usGfsacuAfgGfCfauguUfcAfgugaasgsg | 3882 | CCUUCACUGAACAUGCCUAGUCC | 4068 |
| AD-159855 | ascscaacUfaUfCfCfaaguguuauaL96 | 3697 | usAfsuaaCfaCfUfuggaUfaGfuuggususg | 3883 | CAACCAACUAUCCAAGUGUUAUA | 4069 |
| AD-159864 | cscsaaguGfuUfAfUfaccaacuaaaL96 | 3698 | usUfsuagUfuGfGfuauaAfcAfcuuggsasu | 3884 | AUCCAAGUGUUAUACCAACUAAA | 4070 |
| AD-159488 | asgsuaauAfuUfUfUfaagauggacuL96 | 3699 | asGfsuccAfuCfUfuaaaAfuAfuuacusgsc | 3885 | GCAGUAAUAUUUUAAGAUGGACU | 4071 |
| AD-159553 | asasaaucCfaCfAfGfcuauauccuaL96 | 3700 | usAfsggaUfaUfAfgcugUfgGfauuuusasc | 3886 | GUAAAAUCCACAGCUAUAUCCUG | 4072 |
| AD-159703 | uscscuucAfcUfGfAfacaugccuaaL96 | 3701 | usUfsaggCfaUfGfuucaGfuGfaaggasgsc | 3887 | GCUCCUUCACUGAACAUGCCUAG | 4073 |
| AD-159708 | csascugaAfcAfUfGfccuaguccaaL96 | 3702 | usUfsggaCfuAfGfgcauGfuUfcagugsasa | 3888 | UUCACUGAACAUGCCUAGUCCAA | 4074 |
| AD-159866 | asasguguUfaUfAfCfcaacuaaaacL96 | 3703 | gsUfsuuuAfgUfUfgguaUfaAfcacuusgsg | 3889 | CCAAGUGUUAUACCAACUAAAAC | 4075 |
| AD-159232 | ususccacCfaUfGfAfuuaagggucuL96 | 3704 | asGfsaccCfuUfAfaucaUfgGfuggaasasc | 3890 | GUUUCCACCAUGAUUAAGGGUCU | 4076 |
| AD-159712 | gsasacauGfcCfUfAfguccaacauuL96 | 3705 | asAfsuguUfgGfAfcuagGfcAfuguucsasg | 3891 | CUGAACAUGCCUAGUCCAACAUU | 4077 |
| AD-159808 | asuscaguAfgUfGfUfacauuaccauL96 | 3706 | asUfsgguAfaUfGfuacaCfuAfcugausasu | 3892 | AUAUCAGUAGUGUACAUUACCAU | 4078 |
| AD-159862 | asusccaaGfuGfUfUfauaccaacuaL96 | 3707 | usAfsguuGfgUfAfuaacAfcUfuggausasg | 3893 | CUAUCCAAGUGUUAUACCAACUA | 4079 |
| AD-158503 | cscsaaguCfcAfAfUfauggcaacuaL96 | 3708 | usAfsguuGfcCfAfuauuGfgAfcuuggsasa | 3894 | UUCCAAGUCCAAUAUGGCAACUC | 4080 |
| AD-159412 | csasuuucAfcUfGfUfcuaggcuacaL96 | 3709 | usGfsuagCfcUfAfgacaGfuGfaaaugsasu | 3895 | AUCAUUUCACUGUCUAGGCUACA | 4081 |
| AD-159558 | cscsacagCfuAfUfAfuccugaugcuL96 | 3710 | asGfscauCfaGfGfauauAfgCfuguggsasu | 3896 | AUCCACAGCUAUAUCCUGAUGCU | 4082 |
| AD-159705 | csusucacUfgAfAfCfaugccuaguaL96 | 3711 | usAfscuaGfgCfAfuguuCfaGfugaagsgsa | 3897 | UCCUUCACUGAACAUGCCUAGUC | 4083 |
| AD-159113 | gsusgguuGfaGfAfGfugcuuaugaaL96 | 3712 | usUfscauAfaGfCfacucUfcAfaccacscsu | 3898 | AGGUGGUUGAGAGUGCUUAUGAG | 4084 |
| AD-159806 | asusaucaGfuAfGfUfguacauuacaL96 | 3713 | usGfsuaaUfgUfAfcacuAfcUfgauausasg | 3899 | CUAUAUCAGUAGUGUACAUUACC | 4085 |
| AD-159853 | csasaccaAfcUfAfUfccaaguguuaL96 | 3714 | usAfsacaCfuUfGfgauaGfuUfgguugscsa | 3900 | UGCAACCAACUAUCCAAGUGUUA | 4086 |
| AD-159182 | gscsagauUfuGfGfCfagagaguauaL96 | 3715 | usAfsuacUfcUfCfugccAfaAfucugcsusa | 3901 | UAGCAGAUUUGGCAGAGAGUAUA | 4087 |
| AD-159702 | csusccuuCfaCfUfGfaacaugccuaL96 | 3716 | usAfsggcAfuGfUfucagUfgAfaggagscsc | 3902 | GGCUCCUUCACUGAACAUGCCUA | 4088 |
| AD-159715 | csasugccUfaGfUfCfcaacauuuuuL96 | 3717 | asAfsaaaUfgUfUfggacUfaGfgcaugsusu | 3903 | AACAUGCCUAGUCCAACAUUUUU | 4089 |
| AD-158575 | usgsccauCfaGfUfAfucuuaaugaaL96 | 3718 | usUfscauUfaAfGfauacUfgAfuggcascsa | 3904 | UGUGCCAUCAGUAUCUUAAUGAA | 4090 |
| AD-158576 | gscscaucAfgUfAfUfcuuaaugaaaL96 | 3719 | usUfsucaUfuAfAfgauaCfuGfauggcsasc | 3905 | GUGCCAUCAGUAUCUUAAUGAAG | 4091 |
| AD-158684 | ususagaaCfaCfCfAfaagauugucuL96 | 3720 | asGfsacaAfuCfUfuuggUfgUfucuaasgsg | 3906 | CCUUAGAACACCAAAGAUUGUCU | 4092 |
| AD-159410 | asuscauuUfcAfCfUfgucuaggcuaL96 | 3721 | usAfsgccUfaGfAfcaguGfaAfaugausasu | 3907 | AUAUCAUUUCACUGUCUAGGCUA | 4093 |
| AD-159416 | uscsacugUfcUfAfGfgcuacaacaaL96 | 3722 | usUfsguuGfuAfGfccuaGfaCfagugasasa | 3908 | UUUCACUGUCUAGGCUACAACAG | 4094 |
| AD-159857 | csasacuaUfcCfAfAfguguuauacaL96 | 3723 | usGfsuauAfaCfAfcuugGfaUfaguugsgsu | 3909 | ACCAACUAUCCAAGUGUUAUACC | 4095 |
| AD-158497 | ususgguuCfcAfAfGfuccaauaugaL96 | 3724 | usCfsauaUfuGfGfacuuGfgAfaccaasasa | 3910 | UUUUGGUUCCAAGUCCAAUAUGG | 4096 |
| AD-159124 | usgscuuaUfgAfGfGfugaucaaacuL96 | 3725 | asGfsuuuGfaUfCfaccuCfaUfaagcascsu | 3911 | AGUGCUUAUGAGGUGAUCAAACU | 4097 |
| AD-159312 | uscsucagAfcCfUfUfgugaaggugaL96 | 3726 | usCfsaccUfuCfAfcaagGfuCfugagasusu | 3912 | AAUCUCAGACCUUGUGAAGGUGA | 4098 |
| AD-159552 | usasaaauCfcAfCfAfgcuauauccuL96 | 3727 | asGfsgauAfuAfGfcuguGfgAfuuuuascsa | 3913 | UGUAAAAUCCACAGCUAUAUCCU | 4099 |
| AD-159704 | cscsuucaCfuGfAfAfcaugccuaguL96 | 3728 | asCfsuagGfcAfUfguucAfgUfgaaggsasg | 3914 | CUCCUUCACUGAACAUGCCUAGU | 4100 |
| AD-159737 | gsgsgaucCfaGfUfGfuauaaauccaL96 | 3729 | usGfsgauUfuAfUfacacUfgGfaucccsasg | 3915 | CUGGGAUCCAGUGUAUAAAUCCA | 4101 |
| AD-159869 | csasauaaAfcCfUfUfgaacagugaaL96 | 3730 | usUfscacUfgUfUfcaagGfuUfuauugsgsg | 3916 | CCCAAUAAACCUUGAACAGUGAC | 4102 |
| AD-158570 | gsgsccugUfgCfCfAfucaguaucuuL96 | 3731 | asAfsgauAfcUfGfauggCfaCfaggccsasu | 3917 | AUGGCCUGUGCCAUCAGUAUCUU | 4103 |
| AD-158618 | ususguugAfuGfUfCfaucgaagacaL96 | 3732 | usGfsucuUfcGfAfugacAfuCfaacaasgsa | 3918 | UCUUGUUGAUGUCAUCGAAGACA | 4104 |
| AD-159184 | asgsauuuGfgCfAfGfagaguauaauL96 | 3733 | asUfsuauAfcUfCfucugCfcAfaaucusgsc | 3919 | GCAGAUUUGGCAGAGAGUAUAAU | 4105 |
| AD-159231 | ususuccaCfcAfUfGfauuaaggguaL96 | 3734 | usAfscccUfuAfAfucauGfgUfggaaascsu | 3920 | AGUUUCCACCAUGAUUAAGGGUC | 4106 |
| AD-159423 | csusaggcUfaCfAfAfcaggauucuaL96 | 3735 | usAfsgaaUfcCfUfguugUfaGfccuagsasc | 3921 | GUCUAGGCUACAACAGGAUUCUA | 4107 |
| AD-159446 | usgsgaggUfuGfUfGfcauguugucaL96 | 3736 | usGfsacaAfcAfUfgcacAfaCfcuccascsc | 3922 | GGUGGAGGUUGUGCAUGUUGUCC | 4108 |
| AD-159701 | gscsuccuUfcAfCfUfgaacaugccuL96 | 3737 | asGfsgcaUfgUfUfcaguGfaAfggagcscsa | 3923 | UGGCUCCUUCACUGAACAUGCCU | 4109 |
| AD-158494 | csusuuugGfuUfCfCfaaguccaauaL96 | 3738 | usAfsuugGfaCfUfuggaAfcCfaaaagsgsa | 3924 | UCCUUUUGGUUCCAAGUCCAAUA | 4110 |
| AD-158571 | gscscuguGfcCfAfUfcaguaucuuaL96 | 3739 | usAfsagaUfaCfUfgaugGfcAfcaggcscsa | 3925 | UGGCCUGUGCCAUCAGUAUCUUA | 4111 |
| AD-159125 | gscsuuauGfaGfGfUfgaucaaacuaL96 | 3740 | usAfsguuUfgAfUfcaccUfcAfuaagcsasc | 3926 | GUGCUUAUGAGGUGAUCAAACUC | 4112 |
| AD-159126 | csusuaugAfgGfUfGfaucaaacucaL96 | 3741 | usGfsaguUfuGfAfucacCfuCfauaagscsa | 3927 | UGCUUAUGAGGUGAUCAAACUCA | 4113 |
| AD-159287 | cscsuugcAfuUfUfUfgggacagaauL96 | 3742 | asUfsucuGfuCfCfcaaaAfuGfcaaggsasa | 3928 | UUCCUUGCAUUUUGGGACAGAAU | 4114 |
| AD-158499 | gsgsuuccAfaGfUfCfcaauauggcaL96 | 3743 | usGfsccaUfaUfUfggacUfuGfgaaccsasa | 3929 | UUGGUUCCAAGUCCAAUAUGGCA | 4115 |
| AD-159417 | csascuguCfuAfGfGfcuacaacagaL96 | 3744 | usCfsuguUfgUfAfgccuAfgAfcagugsasa | 3930 | UUCACUGUCUAGGCUACAACAGG | 4116 |
| AD-159418 | ascsugucUfaGfGfCfuacaacaggaL96 | 3745 | usCfscugUfuGfUfagccUfaGfacagusgsa | 3931 | UCACUGUCUAGGCUACAACAGGA | 4117 |
| AD-158550 | asasuaagAfuUfAfCfaguuguuggaL96 | 3746 | usCfscaaCfaAfCfuguaAfuCfuuauuscsu | 3932 | AGAAUAAGAUUACAGUUGUUGGG | 4118 |
| AD-159116 | gsusugagAfgUfGfCfuuaugagguaL96 | 3747 | usAfsccuCfaUfAfagcaCfuCfucaacscsa | 3933 | UGGUUGAGAGUGCUUAUGAGGUG | 4119 |
| AD-159421 | gsuscuagGfcUfAfCfaacaggauuaL96 | 3748 | usAfsaucCfuGfUfuguaGfcCfuagacsasg | 3934 | CUGUCUAGGCUACAACAGGAUUC | 4120 |
| AD-159422 | uscsuaggCfuAfCfAfacaggauucuL96 | 3749 | asGfsaauCfcUfGfuuguAfgCfcuagascsa | 3935 | UGUCUAGGCUACAACAGGAUUCU | 4121 |
| AD-159445 | gsusggagGfuUfGfUfgcauguuguaL96 | 3750 | usAfscaaCfaUfGfcacaAfcCfuccacscsu | 3936 | AGGUGGAGGUUGUGCAUGUUGUC | 4122 |
| AD-159130 | usgsagguGfaUfCfAfaacucaaagaL96 | 3751 | usCfsuuuGfaGfUfuugaUfcAfccucasusa | 3937 | UAUGAGGUGAUCAAACUCAAAGG | 4123 |
| AD-159134 | gsusgaucAfaAfCfUfcaaaggcuaaL96 | 3752 | usUfsagcCfuUfUfgaguUfuGfaucacscsu | 3938 | AGGUGAUCAAACUCAAAGGCUAC | 4124 |
| AD-159343 | usgsaggaAfgAfGfGfcccguuugaaL96 | 3753 | usUfscaaAfcGfGfgccuCfuUfccucasgsa | 3939 | UCUGAGGAAGAGGCCCGUUUGAA | 4125 |
| AD-159105 | ascsaagcAfgGfUfGfguugagaguaL96 | 3754 | usAfscucUfcAfAfccacCfuGfcuugusgsa | 3940 | UCACAAGCAGGUGGUUGAGAGUG | 4126 |
| AD-159183 | csasgauuUfgGfCfAfgagaguauaaL96 | 3755 | usUfsauaCfuCfUfcugcCfaAfaucugscsu | 3941 | AGCAGAUUUGGCAGAGAGUAUAA | 4127 |
| AD-159123 | gsusgcuuAfuGfAfGfgugaucaaacL96 | 3756 | gsUfsuugAfuCfAfccucAfuAfagcacsusc | 3942 | GAGUGCUUAUGAGGUGAUCAAAC | 4128 |
| AD-159181 | asgscagaUfuUfGfGfcagagaguauL96 | 3757 | asUfsacuCfuCfUfgccaAfaUfcugcusasc | 3943 | GUAGCAGAUUUGGCAGAGAGUAU | 4129 |
| AD-159186 | asusuuggCfaGfAfGfaguauaaugaL96 | 3758 | usCfsauuAfuAfCfucucUfgCfcaaauscsu | 3944 | AGAUUUGGCAGAGAGUAUAAUGA | 4130 |
| AD-159187 | ususuggcAfgAfGfAfguauaaugaaL96 | 3759 | usUfscauUfaUfAfcucuCfuGfccaaasusc | 3945 | GAUUUGGCAGAGAGUAUAAUGAA | 4131 |
| AD-159288 | csusugcaUfuUfUfGfggacagaauaL96 | 3760 | usAfsuucUfgUfCfccaaAfaUfgcaagsgsa | 3946 | UCCUUGCAUUUUGGGACAGAAUG | 4132 |
| AD-159306 | asusggaaUfcUfCfAfgaccuugugaL96 | 3761 | usCfsacaAfgGfUfcugaGfaUfuccaususc | 3947 | GAAUGGAAUCUCAGACCUUGUGA | 4133 |
| AD-159559 | csascagcUfaUfAfUfccugaugcuaL96 | 3762 | usAfsgcaUfcAfGfgauaUfaGfcugugsgsa | 3948 | UCCACAGCUAUAUCCUGAUGCUG | 4134 |
| AD-159344 | gsasggaaGfaGfGfCfccguuugaaaL96 | 3763 | usUfsucaAfaCfGfggccUfcUfuccucsasg | 3949 | CUGAGGAAGAGGCCCGUUUGAAG | 4135 |
| AD-159341 | uscsugagGfaAfGfAfggcccguuuaL96 | 3764 | usAfsaacGfgGfCfcucuUfcCfucagasasg | 3950 | CUUCUGAGGAAGAGGCCCGUUUG | 4136 |
| AD-159729 | csascaucCfuGfGfGfauccaguguaL96 | 3765 | usAfscacUfgGfAfucccAfgGfaugugsasc | 3951 | GUCACAUCCUGGGAUCCAGUGUA | 4137 |
| AD-158674 | asgsccuuUfuCfCfUfuagaacaccaL96 | 3766 | usGfsgugUfuCfUfaaggAfaAfaggcusgsc | 3952 | GCAGCCUUUUCCUUAGAACACCA | 4138 |
| AD-159604 | uscsaacuGfgUfUfAfgugugaaauaL96 | 3767 | usAfsuuuCfaCfAfcuaaCfcAfguugasasg | 3953 | CUUCAACUGGUUAGUGUGAAAUA | 4139 |

TABLE 4
Modified Human/Mouse/Cyno/Rat, Mouse, Mouse/Rat, and Human/Cyno Cross-Reactive HAO1 iRNA Sequences

| Duplex | | | | | |
|---|---|---|---|---|---|
| Name | Sense Strand Sequence 5′ to 3′ | SEQ ID NO: | Antisense Strand Sequence 5′ to 3′ | SEQ ID NO: | Species |
| AD-62933 | GfsasAfuGfuGfaAfAfGfuCfaUfcGfaCfaAfL96 | 4140 | usUfsgUfcGfaUfgAfcuuUfcAfcAfuUfcsusg | 89 | Hs/Mm |
| AD-62939 | UfsusUfuCfaAfuGfGfGfuGfuCfcUfaGfgAfL96 | 4141 | usCfscUfaGfgAfcAfcccAfuUfgAfaAfasgsu | 90 | Hs/Mm |
| AD-62944 | GfsasAfaGfuCfaUfCfGfaCfaAfgAfcAfuUfL96 | 4142 | asAfsuGfuCfuUfgUfcgaUfgAfcUfuUfcsasc | 91 | Hs/Mm |
| AD-62949 | UfscsAfuCfgAfcAfAfGfaCfaUfuGfgUfgAfL96 | 4143 | usCfsaCfcAfaUfgUfcuuGfuCfgAfuGfascsu | 92 | Hs/Mm |
| AD-62954 | UfsusUfcAfaUfgGfGfUfgUfcCfuAfgGfaAfL96 | 4144 | usUfscCfuAfgGfaCfaccCfaUfuGfaAfasasg | 93 | Hs/Mm |
| AD-62959 | AfsasUfgGfgUfgUfCfCfuAfgGfaAfcCfuUfL96 | 4145 | asAfsgGfuUfcCfuAfggaCfaCfcCfaUfusgsa | 94 | Hs/Mm |
| AD-62964 | GfsasCfaGfuGfcAfCfAfaUfaUfuUfuCfcAfL96 | 4146 | usGfsgAfaAfaUfaUfuguGfcAfcUfgUfcsasg | 95 | Hs/Mm |
| AD-62969 | AfscsUfuUfuCfaAfUfGfgGfuGfuCfcUfaAfL96 | 4147 | usUfsaGfgAfcAfcCfcauUfgAfaAfaGfuscsa | 96 | Hs/Mm |
| AD-62934 | AfsasGfuCfaUfcGfAfCfaAfgAfcAfuUfgAfL96 | 4148 | usCfsaAfuGfuCfuUfgucGfaUfgAfcUfususc | 97 | Hs/Mm |
| AD-62940 | AfsusCfgAfcAfaGfAfCfaUfuGfgUfgAfgAfL96 | 4149 | usCfsuCfaCfcAfaUfgucUfuGfuCfgAfusgsa | 98 | Hs/Mm |
| AD-62945 | GfsgsGfaGfaAfaGfGfUfgUfuCfaAfgAfuAfL96 | 4150 | usAfsuCfuUfgAfaCfaccUfuUfcUfcCfcscsc | 99 | Hs/Mm |
| AD-62950 | CfsusUfuUfcAfaUfGfGfgUfgUfcCfuAfgAfL96 | 4311 | usCfsuAfgGfaCfaCfccaUfuGfaAfaAfgsusc | 100 | Hs/Mm |
| AD-62955 | UfscsAfaUfgGfgUfGfUfcCfuAfgGfaAfcAfL96 | 4312 | usGfsuUfcCfuAfgGfacaCfcCfaUfuGfasasa | 101 | Hs/Mm |
| AD-62960 | UfsusGfaCfuUfuUfCfAfaUfgGfgUfgUfcAfL96 | 4313 | usGfsaCfaCfcCfaUfugaAfaAfgUfcAfasasa | 102 | Hs/Mm |
| AD-62965 | AfsasAfgUfcAfuCfGfAfcAfaGfaCfaUfuAfL96 | 4314 | usAfsaUfgUfcUfuGfucgAfuGfaCfuUfuscsa | 103 | Hs/Mm |
| AD-62970 | CfsasGfgGfgGfaGfAfAfaGfgUfgUfuCfaAfL96 | 4315 | usUfsgAfaCfaCfcUfuucUfcCfcCfcUfgsgsa | 104 | Hs/Mm |
| AD-62935 | CfsasUfuGfgUfgAfGfGfaAfaAfaUfcCfuUfL96 | 4316 | asAfsgGfaUfuUfuUfccuCfaCfcAfaUfgsusc | 105 | Hs/Mm |
| AD-62941 | AfscsAfuUfgGfuGfAfGfgAfaAfaAfuCfcUfL96 | 4317 | asGfsgAfuUfuUfuCfcucAfcCfaAfuGfuscsu | 106 | Hs/Mm |
| AD-62946 | AfsgsGfgGfgAfgAfAfAfgGfuGfuUfcAfaAfL96 | 4318 | usUfsuGfaAfcAfcCfuuuCfuCfcCfcCfusgsg | 107 | Hs/Mm |
| AD-62951 | AfsusGfgUfgGfuAfAfUfuUfgUfgAfuUfuUfL96 | 37 | asAfsaAfuCfaCfaAfauuAfcCfaCfcAfuscsc | 108 | Hs |
| AD-62956 | GfsasCfuUfgCfaUfCfCfuGfgAfaAfuAfuAfL96 | 38 | usAfsuAfuUfuCfcAfggaUfgCfaAfgUfcscsa | 109 | Hs |
| AD-62961 | GfsgsAfaGfgGfaAfGfGfuAfgAfaGfuCfuUfL96 | 39 | asAfsgAfcUfuCfuAfccuUfcCfcUfuCfcsasc | 110 | Hs |
| AD-62966 | UfsgsUfcUfuCfuGfUfUfuAfgAfuUfuCfcUfL96 | 40 | asGfsgAfaAfuCfuAfaacAfgAfaGfaCfasgsg | 111 | Hs |
| AD-62971 | CfsusUfuGfgCfuGfUfUfuCfcAfaGfaUfcUfL96 | 41 | asGfsaUfcUfuGfgAfaacAfgCfcAfaAfgsgsa | 112 | Hs |
| AD-62936 | AfsasUfgUfgUfuUfGfGfgCfaAfcGfuCfaUfL96 | 42 | asUfsgAfcGfuUfgCfccaAfaCfaCfaUfususu | 113 | Hs |
| AD-62942 | UfsgsUfgAfcUfgUfGfGfaCfaCfcCfcUfuAfL96 | 43 | usAfsaGfgGfgUfgUfccaCfaGfuCfaCfasasa | 114 | Hs |
| AD-62947 | GfsasUfgGfgGfuGfCfCfaGfcUfaCfuAfuUfL96 | 44 | asAfsuAfgUfaGfcUfggcAfcCfcCfaUfcscsa | 115 | Hs |
| AD-62952 | GfsasAfaAfuGfuGfUfUfuGfgGfcAfaCfgUfL96 | 45 | asCfsgUfuGfcCfcAfaacAfcAfuUfuUfcsasa | 116 | Hs |
| AD-62957 | GfsgsCfuGfuUfuCfCfAfaGfaUfcUfgAfcAfL96 | 46 | usGfsuCfaGfaUfcUfuggAfaAfcAfgCfcsasa | 117 | Hs |
| AD-62962 | UfscsCfaAfcAfaAfAfUfaGfcCfaCfcCfcUfL96 | 47 | asGfsgGfgUfgGfcUfauuUfuGfuUfgGfasasa | 118 | Hs |
| AD-62967 | GfsusCfuUfcUfgUfUfUfaGfaUfuUfcCfuUfL96 | 48 | asAfsgGfaAfaUfcUfaaaCfaGfaAfgAfcsasg | 119 | Hs |
| AD-62972 | UfsgsGfaAfgGfgAfAfGfgUfaGfaAfgUfcUfL96 | 49 | asGfsaCfuUfcUfaCfcuuCfcCfuUfcCfascsa | 120 | Hs |
| AD-62937 | UfscsCfuUfuGfgCfUfGfuUfuCfcAfaGfaUfL96 | 50 | asUfscUfuGfgAfaAfcagCfcAfaAfgGfasusu | 121 | Hs |
| AD-62943 | CfsasUfcUfcUfcAfGfCfuGfgGfaUfgAfuAfL96 | 51 | usAfsuCfaUfcCfcAfgcuGfaGfaGfaUfgsgsg | 122 | Hs |
| AD-62948 | GfsgsGfgUfgCfcAfGfCfuAfcUfaUfuGfaUfL96 | 52 | asUfscAfaUfaGfuAfgcuGfgCfaCfcCfcsasu | 123 | Hs |
| AD-62953 | AfsusGfuGfuUfuGfGfGfcAfaCfgUfcAfuAfL96 | 53 | usAfsuGfaCfgUfuGfcccAfaAfcAfcAfususu | 124 | Hs |
| AD-62958 | CfsusGfuUfuAfgAfUfUfuCfcUfuAfaGfaAfL96 | 54 | usUfscUfuAfaGfgAfaauCfuAfaAfcAfgsasa | 125 | Hs |
| AD-62963 | AfsgsAfaAfgAfaAfUfGfgAfcUfuGfcAfuAfL96 | 55 | usAfsuGfcAfaGfuCfcauUfuCfuUfuCfusasg | 126 | Hs |
| AD-62968 | GfscsAfuCfcUfgGfAfAfaUfaUfaUfuAfaAfL96 | 56 | usUfsuAfaUfaUfaUfuucCfaGfgAfuGfcsasa | 127 | Hs |
| AD-62973 | CfscsUfgUfcAfgAfCfCfaUfgGfgAfaCfuAfL96 | 57 | usAfsgUfuCfcCfaUfgguCfuGfaCfaGfgscsu | 128 | Hs |
| AD-62938 | AfsasAfcAfuGfgUfGfUfgGfaUfgGfgAfuAfL96 | 58 | usAfsuCfcCfaUfcCfacaCfcAfuGfuUfusasa | 129 | Hs |
| AD-62974 | CfsusCfaGfgAfuGfAfAfaAfaUfuUfuGfaAfL96 | 59 | usUfscAfaAfaUfuUfuucAfuCfcUfgAfgsusu | 130 | Hs |
| AD-62978 | CfsasGfcAfuGfuAfUfUfaCfuUfgAfcAfaAfL96 | 60 | usUfsuGfuCfaAfgUfaauAfcAfuGfcUfgsasa | 131 | Hs |
| AD-62982 | UfsasUfgAfaCfaAfCfAfuGfcUfaAfaUfcAfL96 | 61 | usGfsaUfuUfaGfcAfuguUfgUfuCfaUfasasu | 132 | Hs |
| AD-62986 | AfsusAfuAfuCfcAfAfAfuGfuUfuUfaGfgAfL96 | 62 | usCfscUfaAfaAfcAfuuuGfgAfuAfuAfususc | 133 | Hs |
| AD-62990 | CfscsAfgAfuGfgAfAfGfcUfgUfaUfcCfaAfL96 | 63 | usUfsgGfaUfaCfaGfcuuCfcAfuCfuGfgsasa | 134 | Hs |
| AD-62994 | GfsasCfuUfuCfaUfCfCfuGfgAfaAfuAfuAfL96 | 64 | usAfsuAfuUfuCfcAfggaUfgAfaAfgUfcscsa | 135 | Hs |
| AD-62998 | CfscsCfcGfgCfuAfAfUfuUfgUfaUfcAfaUfL96 | 65 | asUfsuGfaUfaCfaAfauuAfgCfcGfgGfgsgsa | 136 | Hs |
| AD-63002 | UfsusAfaAfcAfuGfGfCfuUfgAfaUfgGfgAfL96 | 66 | usCfscCfaUfuCfaAfgccAfuGfuUfuAfascsa | 137 | Hs |
| AD-62975 | AfsasUfgUfgUfuUfAfGfaCfaAfcGfuCfaUfL96 | 67 | asUfsgAfcGfuUfgUfcuaAfaCfaCfaUfususu | 138 | Mm |
| AD-62979 | AfscsUfaAfaGfgAfAfGfaAfuUfcCfgGfuUfL96 | 68 | asAfscCfgGfaAfuUfcuuCfcUfuUfaGfusasu | 139 | Mm |
| AD-62983 | UfsasUfaUfcCfaAfAfUfgUfuUfuAfgGfaUfL96 | 69 | asUfscCfuAfaAfaCfauuUfgGfaUfaUfasusu | 140 | Mm |
| AD-62987 | GfsusGfcGfgAfaAfGfGfcAfcUfgAfuGfuUfL96 | 70 | asAfscAfuCfaGfuGfccuUfuCfcGfcAfcsasc | 141 | Mm |
| AD-62991 | UfsasAfaAfcAfgUfGfGfuUfcUfuAfaAfuUfL96 | 71 | asAfsuUfuAfaGfaAfccaCfuGfuUfuUfasasa | 142 | Mm |
| AD-62995 | AfsusGfaAfaAfaUfUfUfuGfaAfaCfcAfgUfL96 | 72 | asCfsuGfgUfuUfcAfaaaUfuUfuUfcAfuscsc | 143 | Mm |
| AD-62999 | AfsasCfaAfaAfuAfGfCfaAfuCfcCfuUfuUfL96 | 73 | asAfsaAfgGfgAfuUfgcuAfuUfuUfgUfusgsg | 144 | Mm |
| AD-63003 | CfsusGfaAfaCfaGfAfUfcUfgUfcGfaCfuUfL96 | 74 | asAfsgUfcGfaCfaGfaucUfgUfuUfcAfgscsa | 145 | Mm |
| AD-62976 | UfsusGfuUfgCfaAfAfGfgGfcAfuUfuUfgAfL96 | 75 | usCfsaAfaAfuGfcCfcuuUfgCfaAfcAfasusu | 146 | Mm |
| AD-62980 | CfsusCfaUfuGfuUfUfAfuUfaAfcCfuGfuAfL96 | 76 | usAfscAfgGfuUfaAfuaaAfcAfaUfgAfgsasu | 147 | Mm |
| AD-62984 | CfsasAfcAfaAfaUfAfGfcAfaUfcCfcUfuUfL96 | 77 | asAfsaGfgGfaUfuGfcuaUfuUfuGfuUfgsgsa | 148 | Mm |
| AD-62992 | CfsasUfuGfuUfuAfUfUfaAfcCfuGfuAfuUfL96 | 78 | asAfsuAfcAfgGfuUfaauAfaAfcAfaUfgsasg | 149 | Mm |
| AD-62996 | UfsasUfcAfgCfuGfGfGfaAfgAfuAfuCfaAfL96 | 79 | usUfsgAfuAfuCfuUfcccAfgCfuGfaUfasgsa | 150 | Mm |
| AD-63000 | UfsgsUfcCfuAfgGfAfAfcCfuUfuUfaGfaAfL96 | 80 | usUfscUfaAfaAfgGfuucCfuAfgGfaCfascsc | 151 | Mm |
| AD-63004 | UfscsCfaAfcAfaAfAfUfaGfcAfaUfcCfcUfL96 | 81 | asGfsgGfaUfuGfcUfauuUfuGfuUfgGfasasa | 152 | Mm |
| AD-62977 | GfsgsUfgUfgCfgGfAfAfaGfgCfaCfuGfaUfL96 | 82 | asUfscAfgUfgCfcUfuucCfgCfaCfaCfcscsc | 153 | Mm |
| AD-62981 | UfsusGfaAfaCfcAfGfUfaCfuUfuAfuCfaUfL96 | 83 | asUfsgAfuAfaAfgUfacuGfgUfuUfcAfasasa | 154 | Mm |
| AD-62985 | UfsasCfuUfcCfaAfAfGfuCfuAfuAfuAfuAfL96 | 84 | usAfsuAfuAfuAfgAfcuuUfgGfaAfgUfascsu | 155 | Mm |
| AD-62989 | UfscsCfuAfgGfaAfCfCfuUfuUfaGfaAfaUfL96 | 85 | asUfsuUfcUfaAfaAfgguUfcCfuAfgGfascsa | 156 | Mm |
| AD-62993 | CfsusCfcUfgAfgGfAfAfaAfuUfuUfgGfaAfL96 | 86 | usUfscCfaAfaAfuUfuucCfuCfaGfgAfgsasa | 157 | Mm |
| AD-62997 | GfscsUfcCfgGfaAfUfGfuUfgCfuGfaAfaUfL96 | 87 | asUfsuUfcAfgCfaAfcauUfcCfgGfaGfcsasu | 158 | Mm |
| AD-63001 | GfsusGfuUfuGfuGfGfGfgAfgAfcCfaAfuAfL96 | 88 | usAfsuUfgGfuCfuCfcccAfcAfaAfcAfcsasg | 159 | Mm |

TABLE 5
Additional Modified Human/Mouse/Cyno/Rat, Human/Mouse/Rat, Human/Mouse/Cyno, Mouse, Mouse/Rat, and
Human/Cyno Cross-Reactive HAO1 iRNA Sequences

| | | SEQ | | SEQ | |
|---|---|---|---|---|---|
| Duplex | | ID | | ID | |
| Name | Sense Strand Sequence 5′ to 3′ | NO: | Antisense Strand Sequence 5′ to 3′ | NO: | Species |
| AD-62933.2 | GfsasAfuGfuGfaAfAfGfuCfaUfcGfaCfaAfL96 | 4140 | usUfsgUfcGfaUfgAfcuuUfcAfcAfuUfcsusg | 89 | Hs/Mm |
| AD-62939.2 | UfsusUfuCfaAfuGfGfGfuGfuCfcUfaGfgAfL96 | 4141 | usCfscUfaGfgAfcAfcccAfuUfgAfaAfasgsu | 90 | Hs/Mm |
| AD-62944.2 | GfsasAfaGfuCfaUfCfGfaCfaAfgAfcAfuUfL96 | 4142 | asAfsuGfuCfuUfgUfcgaUfgAfcUfuUfcsasc | 91 | Hs/Mm |
| AD-62949.2 | UfscsAfuCfgAfcAfAfGfaCfaUfuGfgUfgAfL96 | 4143 | usCfsaCfcAfaUfgUfcuuGfuCfgAfuGfascsu | 92 | Hs/Mm |
| AD-62954.2 | UfsusUfcAfaUfgGfGfUfgUfcCfuAfgGfaAfL96 | 4144 | usUfscCfuAfgGfaCfaccCfaUfuGfaAfasasg | 93 | Hs/Mm |
| AD-62959.2 | AfsasUfgGfgUfgUfCfCfuAfgGfaAfcCfuUfL96 | 4145 | asAfsgGfuUfcCfuAfggaCfaCfcCfaUfusgsa | 94 | Hs/Mm |
| AD-62964.2 | GfsasCfaGfuGfcAfCfAfaUfaUfuUfuCfcAfL96 | 4146 | usGfsgAfaAfaUfaUfuguGfcAfcUfgUfcsasg | 95 | Hs/Mm |
| AD-62969.2 | AfscsUfuUfuCfaAfUfGfgGfuGfuCfcUfaAfL96 | 4147 | usUfsaGfgAfcAfcCfcauUfgAfaAfaGfuscsa | 96 | Hs/Mm |
| AD-62934.2 | AfsasGfuCfaUfcGfAfCfaAfgAfcAfuUfgAfL96 | 4148 | usCfsaAfuGfuCfuUfgucGfaUfgAfcUfususc | 97 | Hs/Mm |
| AD-62940.2 | AfsusCfgAfcAfaGfAfCfaUfuGfgUfgAfgAfL96 | 4149 | usCfsuCfaCfcAfaUfgucUfuGfuCfgAfusgsa | 98 | Hs/Mm |
| AD-62945.2 | GfsgsGfaGfaAfaGfGfUfgUfuCfaAfgAfuAfL96 | 4150 | usAfsuCfuUfgAfaCfaccUfuUfcUfcCfcscsc | 99 | Hs/Mm |
| AD-62950.2 | CfsusUfuUfcAfaUfGfGfgUfgUfcCfuAfgAfL96 | 4311 | usCfsuAfgGfaCfaCfccaUfuGfaAfaAfgsusc | 100 | Hs/Mm |
| AD-62955.2 | UfscsAfaUfgGfgUfGfUfcCfuAfgGfaAfcAfL96 | 4312 | usGfsuUfcCfuAfgGfacaCfcCfaUfuGfasasa | 101 | Hs/Mm |
| AD-62960.2 | UfsusGfaCfuUfuUfCfAfaUfgGfgUfgUfcAfL96 | 4313 | usGfsaCfaCfcCfaUfugaAfaAfgUfcAfasasa | 102 | Hs/Mm |
| AD-62965.2 | AfsasAfgUfcAfuCfGfAfcAfaGfaCfaUfuAfL96 | 4314 | usAfsaUfgUfcUfuGfucgAfuGfaCfuUfuscsa | 103 | Hs/Mm |
| AD-62970.2 | CfsasGfgGfgGfaGfAfAfaGfgUfgUfuCfaAfL96 | 4315 | usUfsgAfaCfaCfcUfuucUfcCfcCfcUfgsgsa | 104 | Hs/Mm |
| AD-62935.2 | CfsasUfuGfgUfgAfGfGfaAfaAfaUfcCfuUfL96 | 4316 | asAfsgGfaUfuUfuUfccuCfaCfcAfaUfgsusc | 105 | Hs/Mm |
| AD-62941.2 | AfscsAfuUfgGfuGfAfGfgAfaAfaAfuCfcUfL96 | 4317 | asGfsgAfuUfuUfuCfcucAfcCfaAfuGfuscsu | 106 | Hs/Mm |
| AD-62946.2 | AfsgsGfgGfgAfgAfAfAfgGfuGfuUfcAfaAfL96 | 4318 | usUfsuGfaAfcAfcCfuuuCfuCfcCfcCfusgsg | 107 | Hs/Mm |
| AD-62951.2 | AfsusGfgUfgGfuAfAfUfuUfgUfgAfuUfuUfL96 | 37 | asAfsaAfuCfaCfaAfauuAfcCfaCfcAfuscsc | 108 | Hs |
| AD-62956.2 | GfsasCfuUfgCfaUfCfCfuGfgAfaAfuAfuAfL96 | 38 | usAfsuAfuUfuCfcAfggaUfgCfaAfgUfcscsa | 109 | Hs |
| AD-62961.2 | GfsgsAfaGfgGfaAfGfGfuAfgAfaGfuCfuUfL96 | 39 | asAfsgAfcUfuCfuAfccuUfcCfcUfuCfcsasc | 110 | Hs |
| AD-62966.2 | UfsgsUfcUfuCfuGfUfUfuAfgAfuUfuCfcUfL96 | 40 | asGfsgAfaAfuCfuAfaacAfgAfaGfaCfasgsg | 111 | Hs |
| AD-62971.2 | CfsusUfuGfgCfuGfUfUfuCfcAfaGfaUfcUfL96 | 41 | asGfsaUfcUfuGfgAfaacAfgCfcAfaAfgsgsa | 112 | Hs |
| AD-62936.2 | AfsasUfgUfgUfuUfGfGfgCfaAfcGfuCfaUfL96 | 42 | asUfsgAfcGfuUfgCfccaAfaCfaCfaUfususu | 113 | Hs |
| AD-62942.2 | UfsgsUfgAfcUfgUfGfGfaCfaCfcCfcUfuAfL96 | 43 | usAfsaGfgGfgUfgUfccaCfaGfuCfaCfasasa | 114 | Hs |
| AD-62947.2 | GfsasUfgGfgGfuGfCfCfaGfcUfaCfuAfuUfL96 | 44 | asAfsuAfgUfaGfcUfggcAfcCfcCfaUfcscsa | 115 | Hs |
| AD-62952.2 | GfsasAfaAfuGfuGfUfUfuGfgGfcAfaCfgUfL96 | 45 | asCfsgUfuGfcCfcAfaacAfcAfuUfuUfcsasa | 116 | Hs |
| AD-62957.2 | GfsgsCfuGfuUfuCfCfAfaGfaUfcUfgAfcAfL96 | 46 | usGfsuCfaGfaUfcUfuggAfaAfcAfgCfcsasa | 117 | Hs |
| AD-62962.2 | UfscsCfaAfcAfaAfAfUfaGfcCfaCfcCfcUfL96 | 47 | asGfsgGfgUfgGfcUfauuUfuGfuUfgGfasasa | 118 | Hs |
| AD-62967.2 | GfsusCfuUfcUfgUfUfUfaGfaUfuUfcCfuUfL96 | 48 | asAfsgGfaAfaUfcUfaaaCfaGfaAfgAfcsasg | 119 | Hs |
| AD-62972.2 | UfsgsGfaAfgGfgAfAfGfgUfaGfaAfgUfcUfL96 | 49 | asGfsaCfuUfcUfaCfcuuCfcCfuUfcCfascsa | 120 | Hs |
| AD-62937.2 | UfscsCfuUfuGfgCfUfGfuUfuCfcAfaGfaUfL96 | 50 | asUfscUfuGfgAfaAfcagCfcAfaAfgGfasusu | 121 | Hs |
| AD-62943.2 | CfsasUfcUfcUfcAfGfCfuGfgGfaUfgAfuAfL96 | 51 | usAfsuCfaUfcCfcAfgcuGfaGfaGfaUfgsgsg | 122 | Hs |
| AD-62948.2 | GfsgsGfgUfgCfcAfGfCfuAfcUfaUfuGfaUfL96 | 52 | asUfscAfaUfaGfuAfgcuGfgCfaCfcCfcsasu | 123 | Hs |
| AD-62953.2 | AfsusGfuGfuUfuGfGfGfcAfaCfgUfcAfuAfL96 | 53 | usAfsuGfaCfgUfuGfcccAfaAfcAfcAfususu | 124 | Hs |
| AD-62958.2 | CfsusGfuUfuAfgAfUfUfuCfcUfuAfaGfaAfL96 | 54 | usUfscUfuAfaGfgAfaauCfuAfaAfcAfgsasa | 125 | Hs |
| AD-62963.2 | AfsgsAfaAfgAfaAfUfGfgAfcUfuGfcAfuAfL96 | 55 | usAfsuGfcAfaGfuCfcauUfuCfuUfuCfusasg | 126 | Hs |
| AD-62968.2 | GfscsAfuCfcUfgGfAfAfaUfaUfaUfuAfaAfL96 | 56 | usUfsuAfaUfaUfaUfuucCfaGfgAfuGfcsasa | 127 | Hs |
| AD-62973.2 | CfscsUfgUfcAfgAfCfCfaUfgGfgAfaCfuAfL96 | 57 | usAfsgUfuCfcCfaUfgguCfuGfaCfaGfgscsu | 128 | Hs |
| AD-62938.2 | AfsasAfcAfuGfgUfGfUfgGfaUfgGfgAfuAfL96 | 58 | usAfsuCfcCfaUfcCfacaCfcAfuGfuUfusasa | 129 | Hs |
| AD-62974.2 | CfsusCfaGfgAfuGfAfAfaAfaUfuUfuGfaAfL96 | 59 | usUfscAfaAfaUfuUfuucAfuCfcUfgAfgsusu | 130 | Hs |
| AD-62978.2 | CfsasGfcAfuGfuAfUfUfaCfuUfgAfcAfaAfL96 | 60 | usUfsuGfuCfaAfgUfaauAfcAfuGfcUfgsasa | 131 | Hs |
| AD-62982.2 | UfsasUfgAfaCfaAfCfAfuGfcUfaAfaUfcAfL96 | 61 | usGfsaUfuUfaGfcAfuguUfgUfuCfaUfasasu | 132 | Hs |
| AD-62986.2 | AfsusAfuAfuCfcAfAfAfuGfuUfuUfaGfgAfL96 | 62 | usCfscUfaAfaAfcAfuuuGfgAfuAfuAfususc | 133 | Hs |
| AD-62990.2 | CfscsAfgAfuGfgAfAfGfcUfgUfaUfcCfaAfL96 | 63 | usUfsgGfaUfaCfaGfcuuCfcAfuCfuGfgsasa | 134 | Hs |
| AD-62994.2 | GfsasCfuUfuCfaUfCfCfuGfgAfaAfuAfuAfL96 | 64 | usAfsuAfuUfuCfcAfggaUfgAfaAfgUfcscsa | 135 | Hs |
| AD-62998.2 | CfscsCfcGfgCfuAfAfUfuUfgUfaUfcAfaUfL96 | 65 | asUfsuGfaUfaCfaAfauuAfgCfcGfgGfgsgsa | 136 | Hs |
| AD-63002.2 | UfsusAfaAfcAfuGfGfCfuUfgAfaUfgGfgAfL96 | 66 | usCfscCfaUfuCfaAfgccAfuGfuUfuAfascsa | 137 | Hs |
| AD-62975.2 | AfsasUfgUfgUfuUfAfGfaCfaAfcGfuCfaUfL96 | 67 | asUfsgAfcGfuUfgUfcuaAfaCfaCfaUfususu | 138 | Mm |
| AD-62979.2 | AfscsUfaAfaGfgAfAfGfaAfuUfcCfgGfuUfL96 | 68 | asAfscCfgGfaAfuUfcuuCfcUfuUfaGfusasu | 139 | Mm |
| AD-62983.2 | UfsasUfaUfcCfaAfAfUfgUfuUfuAfgGfaUfL96 | 69 | asUfscCfuAfaAfaCfauuUfgGfaUfaUfasusu | 140 | Mm |
| AD-62987.2 | GfsusGfcGfgAfaAfGfGfcAfcUfgAfuGfuUfL96 | 70 | asAfscAfuCfaGfuGfccuUfuCfcGfcAfcsasc | 141 | Mm |
| AD-62991.2 | UfsasAfaAfcAfgUfGfGfuUfcUfuAfaAfuUfL96 | 71 | asAfsuUfuAfaGfaAfccaCfuGfuUfuUfasasa | 142 | Mm |
| AD-62995.2 | AfsusGfaAfaAfaUfUfUfuGfaAfaCfcAfgUfL96 | 72 | asCfsuGfgUfuUfcAfaaaUfuUfuUfcAfuscsc | 143 | Mm |
| AD-62999.2 | AfsasCfaAfaAfuAfGfCfaAfuCfcCfuUfuUfL96 | 73 | asAfsaAfgGfgAfuUfgcuAfuUfuUfgUfusgsg | 144 | Mm |
| AD-63003.2 | CfsusGfaAfaCfaGfAfUfcUfgUfcGfaCfuUfL96 | 74 | asAfsgUfcGfaCfaGfaucUfgUfuUfcAfgscsa | 145 | Mm |
| AD-62976.2 | UfsusGfuUfgCfaAfAfGfgGfcAfuUfuUfgAfL96 | 75 | usCfsaAfaAfuGfcCfcuuUfgCfaAfcAfasusu | 146 | Mm |
| AD-62980.2 | CfsusCfaUfuGfuUfUfAfuUfaAfcCfuGfuAfL96 | 76 | usAfscAfgGfuUfaAfuaaAfcAfaUfgAfgsasu | 147 | Mm |
| AD-62984.2 | CfsasAfcAfaAfaUfAfGfcAfaUfcCfcUfuUfL96 | 77 | asAfsaGfgGfaUfuGfcuaUfuUfuGfuUfgsgsa | 148 | Mm |
| AD-62992.2 | CfsasUfuGfuUfuAfUfUfaAfcCfuGfuAfuUfL96 | 78 | asAfsuAfcAfgGfuUfaauAfaAfcAfaUfgsasg | 149 | Mm |
| AD-62996.2 | UfsasUfcAfgCfuGfGfGfaAfgAfuAfuCfaAfL96 | 79 | usUfsgAfuAfuCfuUfcccAfgCfuGfaUfasgsa | 150 | Mm |
| AD-63000.2 | UfsgsUfcCfuAfgGfAfAfcCfuUfuUfaGfaAfL96 | 80 | usUfscUfaAfaAfgGfuucCfuAfgGfaCfascsc | 151 | Mm |
| AD-63004.2 | UfscsCfaAfcAfaAfAfUfaGfcAfaUfcCfcUfL96 | 81 | asGfsgGfaUfuGfcUfauuUfuGfuUfgGfasasa | 152 | Mm |
| AD-62977.2 | GfsgsUfgUfgCfgGfAfAfaGfgCfaCfuGfaUfL96 | 82 | asUfscAfgUfgCfcUfuucCfgCfaCfaCfcscsc | 153 | Mm |
| AD-62981.2 | UfsusGfaAfaCfcAfGfUfaCfuUfuAfuCfaUfL96 | 83 | asUfsgAfuAfaAfgUfacuGfgUfuUfcAfasasa | 154 | Mm |
| AD-62985.2 | UfsasCfuUfcCfaAfAfGfuCfuAfuAfuAfuAfL96 | 84 | usAfsuAfuAfuAfgAfcuuUfgGfaAfgUfascsu | 155 | Mm |
| AD-62989.2 | UfscsCfuAfgGfaAfCfCfuUfuUfaGfaAfaUfL96 | 85 | asUfsuUfcUfaAfaAfgguUfcCfuAfgGfascsa | 156 | Mm |
| AD-62993.2 | CfsusCfcUfgAfgGfAfAfaAfuUfuUfgGfaAfL96 | 86 | usUfscCfaAfaAfuUfuucCfuCfaGfgAfgsasa | 157 | Mm |
| AD-62997.2 | GfscsUfcCfgGfaAfUfGfuUfgCfuGfaAfaUfL96 | 87 | asUfsuUfcAfgCfaAfcauUfcCfgGfaGfcsasu | 158 | Mm |
| AD-63001.2 | GfsusGfuUfuGfuGfGfGfgAfgAfcCfaAfuAfL96 | 88 | usAfsuUfgGfuCfuCfcccAfcAfaAfcAfcsasg | 159 | Mm |
| AD-62933.1 | GfsasAfuGfuGfaAfAfGfuCfaUfcGfaCfaAfL96 | 160 | usUfsgUfcGfaUfgAfcuuUfcAfcAfuUfcsusg | 277 | |
| AD-65630.1 | Y44gsasAfuGfuGfaAfAfGfuCfaUfcGfaCfaAfL96 | 161 | PusUfsgUfcGfaUfgAfcuuUfcAfcAfuUfcsusg | 278 | |
| AD-65636.1 | gsasauguGfaAfAfGfucauCfgacaaL96 | 162 | usUfsgUfcGfaUfgAfcuuUfcAfcAfuUfcsusg | 279 | |
| AD-65642.1 | gsasauguGfaAfAfGfucaucgacaaL96 | 163 | usUfsgUfcGfaUfgAfcuuUfcAfcAfuUfcsusg | 280 | |
| AD-65647.1 | gsasauguGfaaAfGfucaucgacaaL96 | 164 | usUfsgUfcGfaUfgAfcuuUfcAfcAfuUfcsusg | 281 | |
| AD-65652.1 | gsasauguGfaaaGfucaucGfacaaL96 | 165 | usUfsgUfcGfaUfgAfcuuUfcAfcAfuUfcsusg | 282 | |
| AD-65657.1 | gsasaugugaaaGfucaucGfacaaL96 | 166 | usUfsgUfcGfaUfgAfcuuUfcAfcAfuUfcsusg | 283 | |
| AD-65662.1 | gsasauguGfaaaGfucaucgacaaL96 | 167 | usUfsgUfcGfaUfgAfcuuUfcAfcAfuUfcsusg | 284 | |
| AD-65625.1 | AfsusGfuGfaAfAfGfuCfaUfcGfaCfaAfL96 | 168 | usUfsgUfcGfaUfgAfcuuUfcAfcAfususc | 285 | |
| AD-65631.1 | asusguGfaAfAfGfucaucgacaaL96 | 169 | usUfsgucGfaugacuuUfcAfcaususc | 286 | |
| AD-65637.1 | GfsasAfuGfuGfaAfAfGfuCfaUfcGfaCfaAfL96 | 170 | usUfsgucGfaUfgAfcuuUfcAfcauucsusg | 287 | |
| AD-65643.1 | GfsasAfuGfuGfaAfAfGfuCfaUfcGfaCfaAfL96 | 171 | usUfsgucGfaUfGfacuuUfcAfcauucsusg | 288 | |
| AD-65648.1 | GfsasAfuGfuGfaAfAfGfuCfaUfcGfaCfaAfL96 | 172 | usUfsgucGfaugacuuUfcAfcauucsusg | 289 | |
| AD-65653.1 | GfsasAfuGfuGfaAfAfGfuCfaUfcGfaCfaAfL96 | 173 | usUfsgucGfaugacuuUfcacauucsusg | 290 | |
| AD-65658.1 | GfsasAfuGfuGfaAfAfGfuCfaUfcGfaCfaAfL96 | 174 | usUfsgucgaugacuuUfcacauucsusg | 291 | |
| AD-65663.1 | gsasauguGfaAfAfGfucaucgacaaL96 | 175 | usUfsgucGfaUfgAfcuuUfcAfcauucsusg | 292 | |
| AD-65626.1 | gsasauguGfaAfAfGfucaucgacaaL96 | 176 | usUfsgucGfaUfGfacuuUfcAfcauucsusg | 293 | |
| AD-65638.1 | gsasauguGfaaAfGfucaucgacaaL96 | 177 | usUfsgucGfaUfgAfcuuUfcAfcauucsusg | 294 | |
| AD-65644.1 | gsasauguGfaaAfGfucaucgacaaL96 | 178 | usUfsgucGfaUfGfacuuUfcAfcauucsusg | 295 | |
| AD-65649.1 | gsasauguGfaaAfGfucaucgacaaL96 | 179 | usUfsgucGfaugacuuUfcAfcauucsusg | 296 | |
| AD-65654.1 | gsasaugugaaagucau(Cgn)gacaaL96 | 180 | usUfsgUfcGfaUfgAfcuuUfcAfcAfuUfcsusg | 297 | |
| AD-65659.1 | gsasaugdTgaaagucau(Cgn)gacaaL96 | 181 | usUfsgUfcGfaUfgAfcuuUfcAfcAfuUfcsusg | 298 | |
| AD-65627.1 | gsasaudGugaaadGucau(Cgn)gacaaL96 | 182 | usUfsgUfcGfaUfgAfcuuUfcAfcAfuUfcsusg | 299 | |
| AD-65633.1 | gsasaugdTgaaadGucau(Cgn)gacaaL96 | 183 | usUfsgUfcGfaUfgAfcuuUfcAfcAfuUfcsusg | 300 | |
| AD-65639.1 | gsasaugudGaaadGucau(Cgn)gacaaL96 | 184 | usUfsgUfcGfaUfgAfcuuUfcAfcAfuUfcsusg | 301 | |
| AD-65645.1 | gsasaugugaaadGucaucdGacaaL96 | 185 | usUfsgUfcGfaUfgAfcuuUfcAfcAfuUfcsusg | 302 | |
| AD-65650.1 | gsasaugugaaadGucaucdTacaaL96 | 186 | usUfsgUfcGfaUfgAfcuuUfcAfcAfuUfcsusg | 303 | |
| AD-65655.1 | gsasaugugaaadGucaucY34acaaL96 | 187 | usUfsgUfcGfaUfgAfcuuUfcAfcAfuUfcsusg | 304 | |
| AD-65660.1 | gsasaugugaaadGucadTcdTacaaL96 | 188 | usUfsgUfcGfaUfgAfcuuUfcAfcAfuUfcsusg | 305 | |
| AD-65665.1 | gsasaugugaaadGucaucdGadCaaL96 | 189 | usUfsgUfcGfaUfgAfcuuUfcAfcAfuUfcsusg | 306 | |
| AD-65628.1 | gsasaugugaaadGucaucdTadCaaL96 | 190 | usUfsgUfcGfaUfgAfcuuUfcAfcAfuUfcsusg | 307 | |
| AD-65634.1 | gsasaugugaaadGucaucY34adCaaL96 | 191 | usUfsgUfcGfaUfgAfcuuUfcAfcAfuUfcsusg | 308 | |
| AD-65646.1 | GfsasAfuGfuGfaAfAfGfuCfaUfcGfaCfaAfL96 | 192 | usdTsgucgaugdAcuudTcacauucsusg | 309 | |
| AD-65656.1 | GfsasAfuGfuGfaAfAfGfuCfaUfcGfaCfaAfL96 | 193 | usUsgucgaugacuudTcacauucsusg | 310 | |
| AD-65661.1 | GfsasAfuGfuGfaAfAfGfuCfaUfcGfaCfaAfL96 | 194 | usdTsgucdGaugacuudTcacauucsusg | 311 | |
| AD-65666.1 | GfsasAfuGfuGfaAfAfGfuCfaUfcGfaCfaAfL96 | 195 | usUsgucdGaugacuudTcacauucsusg | 312 | |
| AD-65629.1 | GfsasAfuGfuGfaAfAfGfuCfaUfcGfaCfaAfL96 | 196 | usdTsgucgaugacuudTcdAcauucsusg | 313 | |
| AD-65635.1 | GfsasAfuGfuGfaAfAfGfuCfaUfcGfaCfaAfL96 | 197 | usdTsgucdGaugacuudTcdAcauucsusg | 314 | |
| AD-65641.1 | gsasaugugaaadGucau(Cgn)gacaaL96 | 198 | usdTsgucgaugdAcuudTcacauucsusg | 315 | |
| AD-62994.1 | GfsasCfuUfuCfaUfCfCfuGfgAfaAfuAfuAfL96 | 199 | usAfsuAfuUfuCfcAfggaUfgAfaAfgUfcscsa | 316 | |
| AD-65595.1 | gsascuuuCfaUfCfCfuggaAfauauaL96 | 200 | usAfsuAfuUfuCfcAfggaUfgAfaAfgUfcscsa | 317 | |
| AD-65600.1 | gsascuuuCfaUfCfCfuggaaauauaL96 | 201 | usAfsuAfuUfuCfcAfggaUfgAfaAfgUfcscsa | 318 | |
| AD-65610.1 | gsascuuuCfaucCfuggaaAfuauaL96 | 202 | usAfsuAfuUfuCfcAfggaUfgAfaAfgUfcscsa | 319 | |
| AD-65615.1 | gsascuuucaucCfuggaaAfuauaL96 | 203 | usAfsuAfuUfuCfcAfggaUfgAfaAfgUfcscsa | 320 | |
| AD-65620.1 | gsascuuuCfaucCfuggaaauauaL96 | 204 | usAfsuAfuUfuCfcAfggaUfgAfaAfgUfcscsa | 321 | |
| AD-65584.1 | CfsusUfuCfaUfCfCfuGfgAfaAfuAfuAfL96 | 205 | usAfsuAfuUfuCfcAfggaUfgAfaAfgsusc | 322 | |
| AD-65590.1 | csusuuCfaUfCfCfuggaaauauaL96 | 206 | usAfsuauUfuccaggaUfgAfaagsusc | 323 | |
| AD-65596.1 | GfsasCfuUfuCfaUfCfCfuGfgAfaAfuAfuAfL96 | 207 | usAfsuauUfuCfcAfggaUfgAfaagucscsa | 324 | |
| AD-65601.1 | GfsasCfuUfuCfaUfCfCfuGfgAfaAfuAfuAfL96 | 208 | usAfsuauUfuCfCfaggaUfgAfaagucscsa | 325 | |
| AD-65606.1 | GfsasCfuUfuCfaUfCfCfuGfgAfaAfuAfuAfL96 | 209 | usAfsuauUfuccaggaUfgAfaagucscsa | 326 | |
| AD-65611.1 | GfsasCfuUfuCfaUfCfCfuGfgAfaAfuAfuAfL96 | 210 | usAfsuauUfuccaggaUfgaaagucscsa | 327 | |
| AD-65616.1 | GfsasCfuUfuCfaUfCfCfuGfgAfaAfuAfuAfL96 | 211 | usAfsuauuuccaggaUfgaaagucscsa | 328 | |
| AD-65621.1 | gsascuuuCfaUfCfCfuggaaauauaL96 | 212 | usAfsuauUfuCfcAfggaUfgAfaagucscsa | 329 | |
| AD-65585.1 | gsascuuuCfaUfCfCfuggaaauauaL96 | 213 | usAfsuauUfuCfCfaggaUfgAfaagucscsa | 330 | |
| AD-65591.1 | gsascuuuCfaUfCfCfuggaaauauaL96 | 214 | usAfsuauUfuccaggaUfgAfaagucscsa | 331 | |
| AD-65597.1 | gsascuuuCfauCfCfuggaaauauaL96 | 215 | usAfsuauUfuCfcAfggaUfgAfaagucscsa | 332 | |
| AD-65602.1 | gsascuuuCfauCfCfuggaaauauaL96 | 216 | usAfsuauUfuCfCfaggaUfgAfaagucscsa | 333 | |
| AD-65607.1 | gsascuuuCfauCfCfuggaaauauaL96 | 217 | usAfsuauUfuccaggaUfgAfaagucscsa | 334 | |
| AD-65612.1 | gsascuuucauccuggaa(Agn)uauaL96 | 218 | usAfsuAfuUfuCfcAfggaUfgAfaAfgUfcscsa | 335 | |
| AD-65622.1 | gsascuuucaucdCuggaa(Agn)uauaL96 | 219 | usAfsuAfuUfuCfcAfggaUfgAfaAfgUfcscsa | 336 | |
| AD-65586.1 | gsascudTucaucdCuggaa(Agn)uauaL96 | 220 | usAfsuAfuUfuCfcAfggaUfgAfaAfgUfcscsa | 337 | |
| AD-65592.1 | gsascuudTcaucdCuggaa(Agn)uauaL96 | 221 | usAfsuAfuUfuCfcAfggaUfgAfaAfgUfcscsa | 338 | |
| AD-65598.1 | gsascuuudCaucdCuggaa(Agn)uauaL96 | 222 | usAfsuAfuUfuCfcAfggaUfgAfaAfgUfcscsa | 339 | |
| AD-65603.1 | gsascuuucaucdCuggaadAuauaL96 | 223 | usAfsuAfuUfuCfcAfggaUfgAfaAfgUfcscsa | 340 | |
| AD-65608.1 | gsascuuucaucdCuggaadTuauaL96 | 224 | usAfsuAfuUfuCfcAfggaUfgAfaAfgUfcscsa | 341 | |
| AD-65613.1 | gsascuuucaucdCuggaaY34uauaL96 | 225 | usAfsuAfuUfuCfcAfggaUfgAfaAfgUfcscsa | 342 | |
| AD-65618.1 | gsascuuucaucdCuggdAadTuauaL96 | 226 | usAfsuAfuUfuCfcAfggaUfgAfaAfgUfcscsa | 343 | |
| AD-65623.1 | gsascuuucaucdCuggaadTudAuaL96 | 227 | usAfsuAfuUfuCfcAfggaUfgAfaAfgUfcscsa | 344 | |
| AD-65587.1 | gsascuuucaucdCuggaa(Agn)udAuaL96 | 228 | usAfsuAfuUfuCfcAfggaUfgAfaAfgUfcscsa | 345 | |
| AD-65593.1 | gsascuudTcaucdCuggaadAudAuaL96 | 229 | usAfsuAfuUfuCfcAfggaUfgAfaAfgUfcscsa | 346 | |
| AD-65599.1 | GfsasCfuUfuCfaUfCfCfuGfgAfaAfuAfuAfL96 | 230 | usdAsuauuuccdAggadTgaaagucscsa | 347 | |
| AD-65604.1 | GfsasCfuUfuCfaUfCfCfuGfgAfaAfuAfuAfL96 | 231 | usdAsuauuuccaggadTgaaagucscsa | 348 | |
| AD-65609.1 | GfsasCfuUfuCfaUfCfCfuGfgAfaAfuAfuAfL96 | 232 | usAsuauuuccaggadTgaaagucscsa | 349 | |
| AD-65614.1 | GfsasCfuUfuCfaUfCfCfuGfgAfaAfuAfuAfL96 | 233 | usdAsuaudTuccaggadTgaaagucscsa | 350 | |
| AD-65619.1 | GfsasCfuUfuCfaUfCfCfuGfgAfaAfuAfuAfL96 | 234 | usAsuaudTuccaggadTgaaagucscsa | 351 | |
| AD-65624.1 | GfsasCfuUfuCfaUfCfCfuGfgAfaAfuAfuAfL96 | 235 | usdAsuauuuccaggadTgdAaagucscsa | 352 | |
| AD-65588.1 | GfsasCfuUfuCfaUfCfCfuGfgAfaAfuAfuAfL96 | 236 | usdAsuaudTuccaggadTgdAaagucscsa | 353 | |
| AD-65594.1 | gsascuuucaucdCuggaa(Agn)uauaL96 | 237 | usdAsuauuuccdAggadTgaaagucscsa | 354 | |
| AD-68309.1 | asgsaaagGfuGfUfUfcaagaugucaL96 | 238 | usGfsacaUfcUfUfgaacAfcCfuuucuscsc | 355 | |
| AD-68303.1 | csasuccuGfgAfAfAfuauauuaacuL96 | 239 | asGfsuuaAfuAfUfauuuCfcAfggaugsasa | 356 | |
| AD-65626.5 | gsasauguGfaAfAfGfucaucgacaaL96 | 240 | usUfsgucGfaUfGfacuuUfcAfcauucsusg | 357 | |
| AD-68295.1 | asgsugcaCfaAfUfAfuuuucccauaL96 | 241 | usAfsuggGfaAfAfauauUfgUfgcacusgsu | 358 | |
| AD-68273.1 | gsasaaguCfaUfCfGfacaagacauuL96 | 242 | asAfsuguCfuUfGfucgaUfgAfcuuucsasc | 359 | |
| AD-68297.1 | asasugugAfaAfGfUfcaucgacaaaL96 | 243 | usUfsuguCfgAfUfgacuUfuCfacauuscsu | 360 | |
| AD-68287.1 | csusggaaAfuAfUfAfuuaacuguuaL96 | 244 | usAfsacaGfuUfAfauauAfuUfuccagsgsa | 361 | |
| AD-68300.1 | asusuuucCfcAfUfCfuguauuauuuL96 | 245 | asAfsauaAfuAfCfagauGfgGfaaaausasu | 362 | |
| AD-68306.1 | usgsucguUfcUfUfUfuccaacaaaaL96 | 246 | usUfsuugUfuGfGfaaaaGfaAfcgacascsc | 363 | |
| AD-68292.1 | asusccugGfaAfAfUfauauuaacuaL96 | 247 | usAfsguuAfaUfAfuauuUfcCfaggausgsa | 364 | |
| AD-68298.1 | gscsauuuUfgAfGfAfggugaugauaL96 | 248 | usAfsucaUfcAfCfcucuCfaAfaaugescsc | 365 | |
| AD-68277.1 | csasggggGfaGfAfAfagguguucaaL96 | 249 | usUfsgaaCfaCfCfuuucUfcCfcccugsgsa | 366 | |
| AD-68289.1 | gsgsaaauAfuAfUfUfaacuguuaaaL96 | 250 | usUfsuaaCfaGfUfuaauAfuAfuuuccsasg | 367 | |
| AD-68272.1 | csasuuggUfgAfGfGfaaaaauccuuL96 | 251 | asAfsggaUfuUfUfuccuCfaCfcaaugsusc | 368 | |
| AD-68282.1 | gsgsgagaAfaGfGfUfguucaagauaL96 | 252 | usAfsucuUfgAfAfcaccUfuUfcucccscsc | 369 | |
| AD-68285.1 | gsgscauuUfuGfAfGfaggugaugauL96 | 253 | asUfscauCfaCfCfucucAfaAfaugccscsu | 370 | |
| AD-68290.1 | usascaaaGfgGfUfGfucguucuuuuL96 | 254 | asAfsaagAfaCfGfacacCfcUfuuguasusu | 371 | |
| AD-68296.1 | usgsggauCfuUfGfGfugucgaaucaL96 | 255 | usGfsauuCfgAfCfaccaAfgAfucccasusu | 372 | |
| AD-68288.1 | csusgacaGfuGfCfAfcaauauuuuaL96 | 256 | usAfsaaaUfaUfUfgugcAfcUfgucagsasu | 373 | |
| AD-68299.1 | csasgugcAfcAfAfUfauuuucccauL96 | 257 | asUfsgggAfaAfAfuauuGfuGfcacugsusc | 374 | |
| AD-68275.1 | ascsuuuuCfaAfUfGfgguguccuaaL96 | 258 | usUfsaggAfcAfCfccauUfgAfaaaguscsa | 375 | |
| AD-68274.1 | ascsauugGfuGfAfGfgaaaaauccuL96 | 259 | asGfsgauUfuUfUfccucAfcCfaauguscsu | 376 | |
| AD-68294.1 | ususgcuuUfuGfAfCfuuuucaaugaL96 | 260 | usCfsauuGfaAfAfagucAfaAfagcaasusg | 377 | |
| AD-68302.1 | csasuuuuGfaGfAfGfgugaugaugaL96 | 261 | usCfsaucAfuCfAfccucUfcAfaaaugscsc | 378 | |
| AD-68279.1 | ususgacuUfuUfCfAfaugggugucaL96 | 262 | usGfsacaCfcCfAfuugaAfaAfgucaasasa | 379 | |
| AD-68304.1 | csgsacuuCfuGfUfUfuuaggacagaL96 | 263 | usCfsuguCfcUfAfaaacAfgAfagucgsasc | 380 | |
| AD-68286.1 | csuscugaGfuGfGfGfugccagaauaL96 | 264 | usAfsuucUfgGfCfacccAfcUfcagagscsc | 381 | |
| AD-68291.1 | gsgsgugcCfaGfAfAfugugaaaguaL96 | 265 | usAfscuuUfcAfCfauucUfgGfcacccsasc | 382 | |
| AD-68283.1 | uscsaaugGfgUfGfUfccuaggaacaL96 | 266 | usGfsuucCfuAfGfgacaCfcCfauugasasa | 383 | |
| AD-68280.1 | asasagucAfuCfGfAfcaagacauuaL96 | 267 | usAfsaugUfcUfUfgucgAfuGfacuuuscsa | 384 | |
| AD-68293.1 | asusuuugAfgAfGfGfugaugaugcaL96 | 268 | usGfscauCfaUfCfaccuCfuCfaaaausgsc | 385 | |
| AD-68276.1 | asuscgacAfaGfAfCfauuggugagaL96 | 269 | usCfsucaCfcAfAfugucUfuGfucgausgsa | 386 | |
| AD-68308.1 | gsgsugccAfgAfAfUfgugaaagucaL96 | 270 | usGfsacuUfuCfAfcauuCfuGfgcaccscsa | 387 | |
| AD-68278.1 | gsascaguGfcAfCfAfauauuuuccaL96 | 271 | usGfsgaaAfaUfAfuuguGfcAfcugucsasg | 388 | |
| AD-68307.1 | ascsaaagAfgAfCfAfcugugcagaaL96 | 272 | usUfscugCfaCfAfguguCfuCfuuuguscsa | 389 | |
| AD-68284.1 | ususuucaAfuGfGfGfuguccuaggaL96 | 273 | usCfscuaGfgAfCfacccAfuUfgaaaasgsu | 390 | |
| AD-68301.1 | cscsguuuCfcAfAfGfaucugacaguL96 | 274 | asCfsuguCfaGfAfucuuGfgAfaacggscsc | 391 | |
| AD-68281.1 | asgsggggAfgAfAfAfgguguucaaaL96 | 275 | usUfsugaAfcAfCfcuuuCfuCfccccusgsg | 392 | |
| AD-68305.1 | asgsucauCfgAfCfAfagacauugguL96 | 276 | asCfscaaUfgUfCfuuguCfgAfugacususu | 393 | |

TABLE 6
Unmodified Human/Mouse/Cyno/Rat, Human/Mouse/Cyno, and Human/Cyno Cross-Reactive HAO1 iRNA Sequences

| | SEQ | | SEQ | | |
|---|---|---|---|---|---|
| Duplex | ID | | ID | | Position in |
| Name | NO: | Sense Strand Sequence 5′ to 3′ | NO: | Antisense Strand Sequence 5′ to 3′ | NM_017545.2 |
| AD-62933 | 394 | GAAUGUGAAAGUCAUCGACAA | 443 | UUGUCGAUGACUUUCACAUUCUG | 1072-1094 |
| AD-62939 | 395 | UUUUCAAUGGGUGUCCUAGGA | 444 | UCCUAGGACACCCAUUGAAAAGU | 1302-1324 |
| AD-62944 | 396 | GAAAGUCAUCGACAAGACAUU | 445 | AAUGUCUUGUCGAUGACUUUCAC | 1078-1100 |
| AD-62949 | 397 | UCAUCGACAAGACAUUGGUGA | 446 | UCACCAAUGUCUUGUCGAUGACU | 1083-1105 |
| AD-62954 | 398 | UUUCAAUGGGUGUCCUAGGAA | 447 | UUCCUAGGACACCCAUUGAAAAG | 1303-1325 |
| AD-62959 | 399 | AAUGGGUGUCCUAGGAACCUU | 448 | AAGGUUCCUAGGACACCCAUUGA | 1307-1329 |
| AD-62964 | 400 | GACAGUGCACAAUAUUUUCCA | 449 | UGGAAAAUAUUGUGCACUGUCAG | 1134-1156_C21A |
| AD-62969 | 401 | ACUUUUCAAUGGGUGUCCUAA | 450 | UUAGGACACCCAUUGAAAAGUCA | 1300-1322_G21A |
| AD-62934 | 402 | AAGUCAUCGACAAGACAUUGA | 451 | UCAAUGUCUUGUCGAUGACUUUC | 1080-1102_G21A |
| AD-62940 | 403 | AUCGACAAGACAUUGGUGAGA | 452 | UCUCACCAAUGUCUUGUCGAUGA | 1085-1107_G21A |
| AD-62945 | 404 | GGGAGAAAGGUGUUCAAGAUA | 453 | UAUCUUGAACACCUUUCUCCCCC | 996-1018_G21A |
| AD-62950 | 405 | CUUUUCAAUGGGUGUCCUAGA | 454 | UCUAGGACACCCAUUGAAAAGUC | 1301-1323_G21A |
| AD-62955 | 406 | UCAAUGGGUGUCCUAGGAACA | 455 | UGUUCCUAGGACACCCAUUGAAA | 1305-1327_C21A |
| AD-62960 | 407 | UUGACUUUUCAAUGGGUGUCA | 456 | UGACACCCAUUGAAAAGUCAAAA | 1297-1319_C21A |
| AD-62965 | 408 | AAAGUCAUCGACAAGACAUUA | 457 | UAAUGUCUUGUCGAUGACUUUCA | 1079-1101_G21A |
| AD-62970 | 409 | CAGGGGGAGAAAGGUGUUCAA | 458 | UUGAACACCUUUCUCCCCCUGGA | 992-1014 |
| AD-62935 | 410 | CAUUGGUGAGGAAAAAUCCUU | 459 | AAGGAUUUUUCCUCACCAAUGUC | 1095-1117 |
| AD-62941 | 411 | ACAUUGGUGAGGAAAAAUCCU | 460 | AGGAUUUUUCCUCACCAAUGUCU | 1094-1116 |
| AD-62946 | 412 | AGGGGGAGAAAGGUGUUCAAA | 461 | UUUGAACACCUUUCUCCCCCUGG | 993-1015_G21A |
| AD-62974 | 413 | CUCAGGAUGAAAAAUUUUGAA | 462 | UUCAAAAUUUUUCAUCCUGAGUU | 563-585 |
| AD-62978 | 414 | CAGCAUGUAUUACUUGACAAA | 463 | UUUGUCAAGUAAUACAUGCUGAA | 1173-1195 |
| AD-62982 | 415 | UAUGAACAACAUGCUAAAUCA | 464 | UGAUUUAGCAUGUUGUUCAUAAU | 53-75 |
| AD-62986 | 416 | AUAUAUCCAAAUGUUUUAGGA | 465 | UCCUAAAACAUUUGGAUAUAUUC | 1679-1701 |
| AD-62990 | 417 | CCAGAUGGAAGCUGUAUCCAA | 466 | UUGGAUACAGCUUCCAUCUGGAA | 156-178 |
| AD-62994 | 418 | GACUUUCAUCCUGGAAAUAUA | 467 | UAUAUUUCCAGGAUGAAAGUCCA | 1341-1363 |
| AD-62998 | 419 | CCCCGGCUAAUUUGUAUCAAU | 468 | AUUGAUACAAAUUAGCCGGGGGA | 29-51 |
| AD-63002 | 420 | UUAAACAUGGCUUGAAUGGGA | 469 | UCCCAUUCAAGCCAUGUUUAACA | 765-787 |
| AD-62975 | 421 | AAUGUGUUUAGACAACGUCAU | 470 | AUGACGUUGUCUAAACACAUUUU | 1388-1410 |
| AD-62979 | 422 | ACUAAAGGAAGAAUUCCGGUU | 471 | AACCGGAAUUCUUCCUUUAGUAU | 1027-1049 |
| AD-62983 | 423 | UAUAUCCAAAUGUUUUAGGAU | 472 | AUCCUAAAACAUUUGGAUAUAUU | 1680-1702 |
| AD-62987 | 424 | GUGCGGAAAGGCACUGAUGUU | 473 | AACAUCAGUGCCUUUCCGCACAC | 902-924 |
| AD-62991 | 425 | UAAAACAGUGGUUCUUAAAUU | 474 | AAUUUAAGAACCACUGUUUUAAA | 1521-1543 |
| AD-62995 | 426 | AUGAAAAAUUUUGAAACCAGU | 475 | ACUGGUUUCAAAAUUUUUCAUCC | 569-591 |
| AD-62999 | 427 | AACAAAAUAGCAAUCCCUUUU | 476 | AAAAGGGAUUGCUAUUUUGUUGG | 1264-1286 |
| AD-63003 | 428 | CUGAAACAGAUCUGUCGACUU | 477 | AAGUCGACAGAUCUGUUUCAGCA | 195-217 |
| AD-62976 | 429 | UUGUUGCAAAGGGCAUUUUGA | 478 | UCAAAAUGCCCUUUGCAACAAUU | 720-742 |
| AD-62980 | 430 | CUCAUUGUUUAUUAACCUGUA | 479 | UACAGGUUAAUAAACAAUGAGAU | 1483-1505 |
| AD-62984 | 431 | CAACAAAAUAGCAAUCCCUUU | 480 | AAAGGGAUUGCUAUUUUGUUGGA | 1263-1285 |
| AD-62992 | 432 | CAUUGUUUAUUAACCUGUAUU | 481 | AAUACAGGUUAAUAAACAAUGAG | 1485-1507 |
| AD-62996 | 433 | UAUCAGCUGGGAAGAUAUCAA | 482 | UUGAUAUCUUCCCAGCUGAUAGA | 670-692 |
| AD-63000 | 434 | UGUCCUAGGAACCUUUUAGAA | 483 | UUCUAAAAGGUUCCUAGGACACC | 1313-1335 |
| AD-63004 | 435 | UCCAACAAAAUAGCAAUCCCU | 484 | AGGGAUUGCUAUUUUGUUGGAAA | 1261-1283 |
| AD-62977 | 436 | GGUGUGCGGAAAGGCACUGAU | 485 | AUCAGUGCCUUUCCGCACACCCC | 899-921 |
| AD-62981 | 437 | UUGAAACCAGUACUUUAUCAU | 486 | AUGAUAAAGUACUGGUUUCAAAA | 579-601 |
| AD-62985 | 438 | UACUUCCAAAGUCUAUAUAUA | 487 | UAUAUAUAGACUUUGGAAGUACU | 75-97_G21A |
| AD-62989 | 439 | UCCUAGGAACCUUUUAGAAAU | 488 | AUUUCUAAAAGGUUCCUAGGACA | 1315-1337_G21U |
| AD-62993 | 440 | CUCCUGAGGAAAAUUUUGGAA | 489 | UUCCAAAAUUUUCCUCAGGAGAA | 603-625_G21A |
| AD-62997 | 441 | GCUCCGGAAUGUUGCUGAAAU | 490 | AUUUCAGCAACAUUCCGGAGCAU | 181-203_C21U |
| AD-63001 | 442 | GUGUUUGUGGGGAGACCAAUA | 491 | UAUUGGUCUCCCCACAAACACAG | 953-975_C21A |

TABLE 7
Additional Unmodified Human/Cyno/Mouse/Rat, Human/Mouse/Cyno, Human/Cyno, and Mouse/Rat HAO1 iRNA
Sequences

| | SEQ ID | Sense strand sequence | SEQ ID | Antisense strand sequence | Position in |
|---|---|---|---|---|---|
| Duplex Name | NO: | 5′ to 3′ | NO: | 5′ to 3′ | NM 017545.2 |
| AD-62933.2 | 394 | GAAUGUGAAAGUCAUCGACAA | 443 | UUGUCGAUGACUUUCACAUUCUG | 1072-1094 |
| AD-62939.2 | 395 | UUUUCAAUGGGUGUCCUAGGA | 444 | UCCUAGGACACCCAUUGAAAAGU | 1302-1324 |
| AD-62944.2 | 396 | GAAAGUCAUCGACAAGACAUU | 445 | AAUGUCUUGUCGAUGACUUUCAC | 1078-1100 |
| AD-62949.2 | 397 | UCAUCGACAAGACAUUGGUGA | 446 | UCACCAAUGUCUUGUCGAUGACU | 1083-1105 |
| AD-62954.2 | 398 | UUUCAAUGGGUGUCCUAGGAA | 447 | UUCCUAGGACACCCAUUGAAAAG | 1303-1325 |
| AD-62959.2 | 399 | AAUGGGUGUCCUAGGAACCUU | 448 | AAGGUUCCUAGGACACCCAUUGA | 1307-1329 |
| AD-62964.2 | 400 | GACAGUGCACAAUAUUUUCCA | 449 | UGGAAAAUAUUGUGCACUGUCAG | 1134-1156_C21A |
| AD-62969.2 | 401 | ACUUUUCAAUGGGUGUCCUAA | 450 | UUAGGACACCCAUUGAAAAGUCA | 1300-1322_G21A |
| AD-62934.2 | 402 | AAGUCAUCGACAAGACAUUGA | 451 | UCAAUGUCUUGUCGAUGACUUUC | 1080-1102_G21A |
| AD-62940.2 | 403 | AUCGACAAGACAUUGGUGAGA | 452 | UCUCACCAAUGUCUUGUCGAUGA | 1085-1107_G21A |
| AD-62945.2 | 404 | GGGAGAAAGGUGUUCAAGAUA | 453 | UAUCUUGAACACCUUUCUCCCCC | 996-1018_G21A |
| AD-62950.2 | 405 | CUUUUCAAUGGGUGUCCUAGA | 454 | UCUAGGACACCCAUUGAAAAGUC | 1301-1323_G21A |
| AD-62955.2 | 406 | UCAAUGGGUGUCCUAGGAACA | 455 | UGUUCCUAGGACACCCAUUGAAA | 1305-1327_C21A |
| AD-62960.2 | 407 | UUGACUUUUCAAUGGGUGUCA | 456 | UGACACCCAUUGAAAAGUCAAAA | 1297-1319_C21A |
| AD-62965.2 | 408 | AAAGUCAUCGACAAGACAUUA | 457 | UAAUGUCUUGUCGAUGACUUUCA | 1079-1101_G21A |
| AD-62970.2 | 409 | CAGGGGGAGAAAGGUGUUCAA | 458 | UUGAACACCUUUCUCCCCCUGGA | 992-1014 |
| AD-62935.2 | 410 | CAUUGGUGAGGAAAAAUCCUU | 459 | AAGGAUUUUUCCUCACCAAUGUC | 1095-1117 |
| AD-62941.2 | 411 | ACAUUGGUGAGGAAAAAUCCU | 460 | AGGAUUUUUCCUCACCAAUGUCU | 1094-1116 |
| AD-62946.2 | 412 | AGGGGGAGAAAGGUGUUCAAA | 461 | UUUGAACACCUUUCUCCCCCUGG | 993-1015_G21A |
| AD-62974.2 | 413 | CUCAGGAUGAAAAAUUUUGAA | 462 | UUCAAAAUUUUUCAUCCUGAGUU | 563-585 |
| AD-62978.2 | 414 | CAGCAUGUAUUACUUGACAAA | 463 | UUUGUCAAGUAAUACAUGCUGAA | 1173-1195 |
| AD-62982.2 | 415 | UAUGAACAACAUGCUAAAUCA | 464 | UGAUUUAGCAUGUUGUUCAUAAU | 53-75 |
| AD-62986.2 | 416 | AUAUAUCCAAAUGUUUUAGGA | 465 | UCCUAAAACAUUUGGAUAUAUUC | 1679-1701 |
| AD-62990.2 | 417 | CCAGAUGGAAGCUGUAUCCAA | 466 | UUGGAUACAGCUUCCAUCUGGAA | 156-178 |
| AD-62994.2 | 418 | GACUUUCAUCCUGGAAAUAUA | 467 | UAUAUUUCCAGGAUGAAAGUCCA | 1341-1363 |
| AD-62998.2 | 419 | CCCCGGCUAAUUUGUAUCAAU | 468 | AUUGAUACAAAUUAGCCGGGGGA | 29-51 |
| AD-63002.2 | 420 | UUAAACAUGGCUUGAAUGGGA | 469 | UCCCAUUCAAGCCAUGUUUAACA | 765-787 |
| AD-62975.2 | 421 | AAUGUGUUUAGACAACGUCAU | 470 | AUGACGUUGUCUAAACACAUUUU | 1388-1410 |
| AD-62979.2 | 422 | ACUAAAGGAAGAAUUCCGGUU | 471 | AACCGGAAUUCUUCCUUUAGUAU | 1027-1049 |
| AD-62983.2 | 423 | UAUAUCCAAAUGUUUUAGGAU | 472 | AUCCUAAAACAUUUGGAUAUAUU | 1680-1702 |
| AD-62987.2 | 424 | GUGCGGAAAGGCACUGAUGUU | 473 | AACAUCAGUGCCUUUCCGCACAC | 902-924 |
| AD-62991.2 | 425 | UAAAACAGUGGUUCUUAAAUU | 474 | AAUUUAAGAACCACUGUUUUAAA | 1521-1543 |
| AD-62995.2 | 426 | AUGAAAAAUUUUGAAACCAGU | 475 | ACUGGUUUCAAAAUUUUUCAUCC | 569-591 |
| AD-62999.2 | 427 | AACAAAAUAGCAAUCCCUUUU | 476 | AAAAGGGAUUGCUAUUUUGUUGG | 1264-1286 |
| AD-63003.2 | 428 | CUGAAACAGAUCUGUCGACUU | 477 | AAGUCGACAGAUCUGUUUCAGCA | 195-217 |
| AD-62976.2 | 429 | UUGUUGCAAAGGGCAUUUUGA | 478 | UCAAAAUGCCCUUUGCAACAAUU | 720-742 |
| AD-62980.2 | 430 | CUCAUUGUUUAUUAACCUGUA | 479 | UACAGGUUAAUAAACAAUGAGAU | 1483-1505 |
| AD-62984.2 | 431 | CAACAAAAUAGCAAUCCCUUU | 480 | AAAGGGAUUGCUAUUUUGUUGGA | 1263-1285 |
| AD-62992.2 | 432 | CAUUGUUUAUUAACCUGUAUU | 481 | AAUACAGGUUAAUAAACAAUGAG | 1485-1507 |
| AD-62996.2 | 433 | UAUCAGCUGGGAAGAUAUCAA | 482 | UUGAUAUCUUCCCAGCUGAUAGA | 670-692 |
| AD-63000.2 | 434 | UGUCCUAGGAACCUUUUAGAA | 483 | UUCUAAAAGGUUCCUAGGACACC | 1313-1335 |
| AD-63004.2 | 435 | UCCAACAAAAUAGCAAUCCCU | 484 | AGGGAUUGCUAUUUUGUUGGAAA | 1261-1283 |
| AD-62977.2 | 436 | GGUGUGCGGAAAGGCACUGAU | 485 | AUCAGUGCCUUUCCGCACACCCC | 899-921 |
| AD-62981.2 | 437 | UUGAAACCAGUACUUUAUCAU | 486 | AUGAUAAAGUACUGGUUUCAAAA | 579-601 |
| AD-62985.2 | 438 | UACUUCCAAAGUCUAUAUAUA | 487 | UAUAUAUAGACUUUGGAAGUACU | 75-97_G21A |
| AD-62989.2 | 439 | UCCUAGGAACCUUUUAGAAAU | 488 | AUUUCUAAAAGGUUCCUAGGACA | 1315-1337_G21U |
| AD-62993.2 | 440 | CUCCUGAGGAAAAUUUUGGAA | 489 | UUCCAAAAUUUUCCUCAGGAGAA | 603-625_G21A |
| AD-62997.2 | 441 | GCUCCGGAAUGUUGCUGAAAU | 490 | AUUUCAGCAACAUUCCGGAGCAU | 181-203_C21U |
| AD-63001.2 | 442 | GUGUUUGUGGGGAGACCAAUA | 491 | UAUUGGUCUCCCCACAAACACAG | 953-975_C21A |
| AD-62951.2 | 492 | AUGGUGGUAAUUUGUGAUUUU | 514 | AAAAUCACAAAUUACCACCAUCC | 1642-1664 |
| AD-62956.2 | 493 | GACUUGCAUCCUGGAAAUAUA | 515 | UAUAUUUCCAGGAUGCAAGUCCA | 1338-1360 |
| AD-62961.2 | 494 | GGAAGGGAAGGUAGAAGUCUU | 516 | AAGACUUCUACCUUCCCUUCCAC | 864-886 |
| AD-62966.2 | 495 | UGUCUUCUGUUUAGAUUUCCU | 517 | AGGAAAUCUAAACAGAAGACAGG | 1506-1528 |
| AD-62971.2 | 496 | CUUUGGCUGUUUCCAAGAUCU | 518 | AGAUCUUGGAAACAGCCAAAGGA | 1109-1131 |
| AD-62936.2 | 497 | AAUGUGUUUGGGCAACGUCAU | 519 | AUGACGUUGCCCAAACACAUUUU | 1385-1407 |
| AD-62942.2 | 498 | UGUGACUGUGGACACCCCUUA | 520 | UAAGGGGUGUCCACAGUCACAAA | 486-508 |
| AD-62947.2 | 499 | GAUGGGGUGCCAGCUACUAUU | 521 | AAUAGUAGCUGGCACCCCAUCCA | 814-836 |
| AD-62952.2 | 500 | GAAAAUGUGUUUGGGCAACGU | 522 | ACGUUGCCCAAACACAUUUUCAA | 1382-1404 |
| AD-62957.2 | 501 | GGCUGUUUCCAAGAUCUGACA | 523 | UGUCAGAUCUUGGAAACAGCCAA | 1113-1135 |
| AD-62962.2 | 502 | UCCAACAAAAUAGCCACCCCU | 524 | AGGGGUGGCUAUUUUGUUGGAAA | 1258-1280 |
| AD-62967.2 | 503 | GUCUUCUGUUUAGAUUUCCUU | 525 | AAGGAAAUCUAAACAGAAGACAG | 1507-1529 |
| AD-62972.2 | 504 | UGGAAGGGAAGGUAGAAGUCU | 526 | AGACUUCUACCUUCCCUUCCACA | 863-885 |
| AD-62937.2 | 505 | UCCUUUGGCUGUUUCCAAGAU | 527 | AUCUUGGAAACAGCCAAAGGAUU | 1107-1129 |
| AD-62943.2 | 506 | CAUCUCUCAGCUGGGAUGAUA | 528 | UAUCAUCCCAGCUGAGAGAUGGG | 662-684 |
| AD-62948.2 | 507 | GGGGUGCCAGCUACUAUUGAU | 529 | AUCAAUAGUAGCUGGCACCCCAU | 817-839 |
| AD-62953.2 | 508 | AUGUGUUUGGGCAACGUCAUA | 530 | UAUGACGUUGCCCAAACACAUUU | 1386-1408_C21A |
| AD-62958.2 | 509 | CUGUUUAGAUUUCCUUAAGAA | 531 | UUCUUAAGGAAAUCUAAACAGAA | 1512-1534_C21A |
| AD-62963.2 | 510 | AGAAAGAAAUGGACUUGCAUA | 532 | UAUGCAAGUCCAUUUCUUUCUAG | 1327-1349_C21A |
| AD-62968.2 | 511 | GCAUCCUGGAAAUAUAUUAAA | 533 | UUUAAUAUAUUUCCAGGAUGCAA | 1343-1365_C21A |
| AD-62973.2 | 512 | CCUGUCAGACCAUGGGAACUA | 534 | UAGUUCCCAUGGUCUGACAGGCU | 308-330_G21A |
| AD-62938.2 | 513 | AAACAUGGUGUGGAUGGGAUA | 535 | UAUCCCAUCCACACCAUGUUUAA | 763-785_C21A |
| AD-62933.1 | 536 | GAAUGUGAAAGUCAUCGACAA | 653 | UUGUCGAUGACUUUCACAUUCUG | 1072-1094 |
| AD-65630.1 | 537 | GAAUGUGAAAGUCAUCGACAA | 654 | UUGUCGAUGACUUUCACAUUCUG | 1072-1094 |
| AD-65636.1 | 538 | GAAUGUGAAAGUCAUCGACAA | 655 | UUGUCGAUGACUUUCACAUUCUG | 1072-1094 |
| AD-65642.1 | 539 | GAAUGUGAAAGUCAUCGACAA | 656 | UUGUCGAUGACUUUCACAUUCUG | 1072-1094 |
| AD-65647.1 | 540 | GAAUGUGAAAGUCAUCGACAA | 657 | UUGUCGAUGACUUUCACAUUCUG | 1072-1094 |
| AD-65652.1 | 541 | GAAUGUGAAAGUCAUCGACAA | 658 | UUGUCGAUGACUUUCACAUUCUG | 1072-1094 |
| AD-65657.1 | 542 | GAAUGUGAAAGUCAUCGACAA | 659 | UUGUCGAUGACUUUCACAUUCUG | 1072-1094 |
| AD-65662.1 | 543 | GAAUGUGAAAGUCAUCGACAA | 660 | UUGUCGAUGACUUUCACAUUCUG | 1072-1094 |
| AD-65625.1 | 544 | AUGUGAAAGUCAUCGACAA | 661 | UUGUCGAUGACUUUCACAUUC | 1072-1094 |
| AD-65631.1 | 545 | AUGUGAAAGUCAUCGACAA | 662 | UUGUCGAUGACUUUCACAUUC | 1072-1094 |
| AD-65637.1 | 546 | GAAUGUGAAAGUCAUCGACAA | 663 | UUGUCGAUGACUUUCACAUUCUG | 1072-1094 |
| AD-65643.1 | 547 | GAAUGUGAAAGUCAUCGACAA | 664 | UUGUCGAUGACUUUCACAUUCUG | 1072-1094 |
| AD-65648.1 | 548 | GAAUGUGAAAGUCAUCGACAA | 665 | UUGUCGAUGACUUUCACAUUCUG | 1072-1094 |
| AD-65653.1 | 549 | GAAUGUGAAAGUCAUCGACAA | 666 | UUGUCGAUGACUUUCACAUUCUG | 1072-1094 |
| AD-65658.1 | 550 | GAAUGUGAAAGUCAUCGACAA | 667 | UUGUCGAUGACUUUCACAUUCUG | 1072-1094 |
| AD-65663.1 | 551 | GAAUGUGAAAGUCAUCGACAA | 668 | UUGUCGAUGACUUUCACAUUCUG | 1072-1094 |
| AD-65626.1 | 552 | GAAUGUGAAAGUCAUCGACAA | 669 | UUGUCGAUGACUUUCACAUUCUG | 1072-1094 |
| AD-65638.1 | 553 | GAAUGUGAAAGUCAUCGACAA | 670 | UUGUCGAUGACUUUCACAUUCUG | 1072-1094 |
| AD-65644.1 | 554 | GAAUGUGAAAGUCAUCGACAA | 671 | UUGUCGAUGACUUUCACAUUCUG | 1072-1094 |
| AD-65649.1 | 555 | GAAUGUGAAAGUCAUCGACAA | 672 | UUGUCGAUGACUUUCACAUUCUG | 1072-1094 |
| AD-65654.1 | 556 | GAAUGUGAAAGUCAUCGACAA | 673 | UUGUCGAUGACUUUCACAUUCUG | 1072-1094 |
| AD-65659.1 | 557 | GAAUGTGAAAGUCAUCGACAA | 674 | UUGUCGAUGACUUUCACAUUCUG | 1072-1094 |
| AD-65627.1 | 558 | GAAUGUGAAAGUCAUCGACAA | 675 | UUGUCGAUGACUUUCACAUUCUG | 1072-1094 |
| AD-65633.1 | 559 | GAAUGTGAAAGUCAUCGACAA | 676 | UUGUCGAUGACUUUCACAUUCUG | 1072-1094 |
| AD-65639.1 | 560 | GAAUGUGAAAGUCAUCGACAA | 677 | UUGUCGAUGACUUUCACAUUCUG | 1072-1094 |
| AD-65645.1 | 561 | GAAUGUGAAAGUCAUCGACAA | 678 | UUGUCGAUGACUUUCACAUUCUG | 1072-1094 |
| AD-65650.1 | 562 | GAAUGUGAAAGUCAUCTACAA | 679 | UUGUCGAUGACUUUCACAUUCUG | 1072-1094 |
| AD-65655.1 | 563 | GAAUGUGAAAGUCAUCACAA | 680 | UUGUCGAUGACUUUCACAUUCUG | 1072-1094 |
| AD-65660.1 | 564 | GAAUGUGAAAGUCATCTACAA | 681 | UUGUCGAUGACUUUCACAUUCUG | 1072-1094 |
| AD-65665.1 | 565 | GAAUGUGAAAGUCAUCGACAA | 682 | UUGUCGAUGACUUUCACAUUCUG | 1072-1094 |
| AD-65628.1 | 566 | GAAUGUGAAAGUCAUCTACAA | 683 | UUGUCGAUGACUUUCACAUUCUG | 1072-1094 |
| AD-65634.1 | 567 | GAAUGUGAAAGUCAUCACAA | 684 | UUGUCGAUGACUUUCACAUUCUG | 1072-1094 |
| AD-65646.1 | 568 | GAAUGUGAAAGUCAUCGACAA | 685 | UTGUCGAUGACUUTCACAUUCUG | 1072-1094 |
| AD-65656.1 | 569 | GAAUGUGAAAGUCAUCGACAA | 686 | UUGUCGAUGACUUTCACAUUCUG | 1072-1094 |
| AD-65661.1 | 570 | GAAUGUGAAAGUCAUCGACAA | 687 | UTGUCGAUGACUUTCACAUUCUG | 1072-1094 |
| AD-65666.1 | 571 | GAAUGUGAAAGUCAUCGACAA | 688 | UUGUCGAUGACUUTCACAUUCUG | 1072-1094 |
| AD-65629.1 | 572 | GAAUGUGAAAGUCAUCGACAA | 689 | UTGUCGAUGACUUTCACAUUCUG | 1072-1094 |
| AD-65635.1 | 573 | GAAUGUGAAAGUCAUCGACAA | 690 | UTGUCGAUGACUUTCACAUUCUG | 1072-1094 |
| AD-65641.1 | 574 | GAAUGUGAAAGUCAUCGACAA | 691 | UTGUCGAUGACUUTCACAUUCUG | 1072-1094 |
| AD-62994.1 | 575 | GACUUUCAUCCUGGAAAUAUA | 692 | UAUAUUUCCAGGAUGAAAGUCCA | 1341-1363 |
| AD-65595.1 | 576 | GACUUUCAUCCUGGAAAUAUA | 693 | UAUAUUUCCAGGAUGAAAGUCCA | 1341-1363 |
| AD-65600.1 | 577 | GACUUUCAUCCUGGAAAUAUA | 694 | UAUAUUUCCAGGAUGAAAGUCCA | 1341-1363 |
| AD-65610.1 | 578 | GACUUUCAUCCUGGAAAUAUA | 695 | UAUAUUUCCAGGAUGAAAGUCCA | 1341-1363 |
| AD-65615.1 | 579 | GACUUUCAUCCUGGAAAUAUA | 696 | UAUAUUUCCAGGAUGAAAGUCCA | 1341-1363 |
| AD-65620.1 | 580 | GACUUUCAUCCUGGAAAUAUA | 697 | UAUAUUUCCAGGAUGAAAGUCCA | 1341-1363 |
| AD-65584.1 | 581 | CUUUCAUCCUGGAAAUAUA | 698 | UAUAUUUCCAGGAUGAAAGUC | 1341-1361 |
| AD-65590.1 | 582 | CUUUCAUCCUGGAAAUAUA | 699 | UAUAUUUCCAGGAUGAAAGUC | 1341-1361 |
| AD-65596.1 | 583 | GACUUUCAUCCUGGAAAUAUA | 700 | UAUAUUUCCAGGAUGAAAGUCCA | 1341-1363 |
| AD-65601.1 | 584 | GACUUUCAUCCUGGAAAUAUA | 701 | UAUAUUUCCAGGAUGAAAGUCCA | 1341-1363 |
| AD-65606.1 | 585 | GACUUUCAUCCUGGAAAUAUA | 702 | UAUAUUUCCAGGAUGAAAGUCCA | 1341-1363 |
| AD-65611.1 | 586 | GACUUUCAUCCUGGAAAUAUA | 703 | UAUAUUUCCAGGAUGAAAGUCCA | 1341-1363 |
| AD-65616.1 | 587 | GACUUUCAUCCUGGAAAUAUA | 704 | UAUAUUUCCAGGAUGAAAGUCCA | 1341-1363 |
| AD-65621.1 | 588 | GACUUUCAUCCUGGAAAUAUA | 705 | UAUAUUUCCAGGAUGAAAGUCCA | 1341-1363 |
| AD-65585.1 | 589 | GACUUUCAUCCUGGAAAUAUA | 706 | UAUAUUUCCAGGAUGAAAGUCCA | 1341-1363 |
| AD-65591.1 | 590 | GACUUUCAUCCUGGAAAUAUA | 707 | UAUAUUUCCAGGAUGAAAGUCCA | 1341-1363 |
| AD-65597.1 | 591 | GACUUUCAUCCUGGAAAUAUA | 708 | UAUAUUUCCAGGAUGAAAGUCCA | 1341-1363 |
| AD-65602.1 | 592 | GACUUUCAUCCUGGAAAUAUA | 709 | UAUAUUUCCAGGAUGAAAGUCCA | 1341-1363 |
| AD-65607.1 | 593 | GACUUUCAUCCUGGAAAUAUA | 710 | UAUAUUUCCAGGAUGAAAGUCCA | 1341-1363 |
| AD-65612.1 | 594 | GACUUUCAUCCUGGAAAUAUA | 711 | UAUAUUUCCAGGAUGAAAGUCCA | 1341-1363 |
| AD-65622.1 | 595 | GACUUUCAUCCUGGAAAUAUA | 712 | UAUAUUUCCAGGAUGAAAGUCCA | 1341-1363 |
| AD-65586.1 | 596 | GACUTUCAUCCUGGAAAUAUA | 713 | UAUAUUUCCAGGAUGAAAGUCCA | 1341-1363 |
| AD-65592.1 | 597 | GACUUTCAUCCUGGAAAUAUA | 714 | UAUAUUUCCAGGAUGAAAGUCCA | 1341-1363 |
| AD-65598.1 | 598 | GACUUUCAUCCUGGAAAUAUA | 715 | UAUAUUUCCAGGAUGAAAGUCCA | 1341-1363 |
| AD-65603.1 | 599 | GACUUUCAUCCUGGAAAUAUA | 716 | UAUAUUUCCAGGAUGAAAGUCCA | 1341-1363 |
| AD-65608.1 | 600 | GACUUUCAUCCUGGAATUAUA | 717 | UAUAUUUCCAGGAUGAAAGUCCA | 1341-1363 |
| AD-65613.1 | 601 | GACUUUCAUCCUGGAAUAUA | 718 | UAUAUUUCCAGGAUGAAAGUCCA | 1341-1363 |
| AD-65618.1 | 602 | GACUUUCAUCCUGGAATUAUA | 719 | UAUAUUUCCAGGAUGAAAGUCCA | 1341-1363 |
| AD-65623.1 | 603 | GACUUUCAUCCUGGAATUAUA | 720 | UAUAUUUCCAGGAUGAAAGUCCA | 1341-1363 |
| AD-65587.1 | 604 | GACUUUCAUCCUGGAAAUAUA | 721 | UAUAUUUCCAGGAUGAAAGUCCA | 1341-1363 |
| AD-65593.1 | 605 | GACUUTCAUCCUGGAAAUAUA | 722 | UAUAUUUCCAGGAUGAAAGUCCA | 1341-1363 |
| AD-65599.1 | 606 | GACUUUCAUCCUGGAAAUAUA | 723 | UAUAUUUCCAGGATGAAAGUCCA | 1341-1363 |
| AD-65604.1 | 607 | GACUUUCAUCCUGGAAAUAUA | 724 | UAUAUUUCCAGGATGAAAGUCCA | 1341-1363 |
| AD-65609.1 | 608 | GACUUUCAUCCUGGAAAUAUA | 725 | UAUAUUUCCAGGATGAAAGUCCA | 1341-1363 |
| AD-65614.1 | 609 | GACUUUCAUCCUGGAAAUAUA | 726 | UAUAUTUCCAGGATGAAAGUCCA | 1341-1363 |
| AD-65619.1 | 610 | GACUUUCAUCCUGGAAAUAUA | 727 | UAUAUTUCCAGGATGAAAGUCCA | 1341-1363 |
| AD-65624.1 | 611 | GACUUUCAUCCUGGAAAUAUA | 728 | UAUAUUUCCAGGATGAAAGUCCA | 1341-1363 |
| AD-65588.1 | 612 | GACUUUCAUCCUGGAAAUAUA | 729 | UAUAUTUCCAGGATGAAAGUCCA | 1341-1363 |
| AD-65594.1 | 613 | GACUUUCAUCCUGGAAAUAUA | 730 | UAUAUUUCCAGGATGAAAGUCCA | 1341-1363 |
| AD-68309.1 | 614 | AGAAAGGUGUUCAAGAUGUCA | 731 | UGACAUCUUGAACACCUUUCUCC | 1001-1022_C21A |
| AD-68303.1 | 615 | CAUCCUGGAAAUAUAUUAACU | 732 | AGUUAAUAUAUUUCCAGGAUGAA | 1349-1370 |
| AD-65626.5 | 616 | GAAUGUGAAAGUCAUCGACAA | 733 | UUGUCGAUGACUUUCACAUUCUG | 1072-1094 |
| AD-68295.1 | 617 | AGUGCACAAUAUUUUCCCAUA | 734 | UAUGGGAAAAUAUUGUGCACUGU | 1139-1160_C21A |
| AD-68273.1 | 618 | GAAAGUCAUCGACAAGACAUU | 735 | AAUGUCUUGUCGAUGACUUUCAC | 1080-1100 |
| AD-68297.1 | 619 | AAUGUGAAAGUCAUCGACAAA | 736 | UUUGUCGAUGACUUUCACAUUCU | 1075-1096_G21A |
| AD-68287.1 | 620 | CUGGAAAUAUAUUAACUGUUA | 737 | UAACAGUUAAUAUAUUUCCAGGA | 1353-1374 |
| AD-68300.1 | 621 | AUUUUCCCAUCUGUAUUAUUU | 738 | AAAUAAUACAGAUGGGAAAAUAU | 1149-1170 |
| AD-68306.1 | 622 | UGUCGUUCUUUUCCAACAAAA | 739 | UUUUGUUGGAAAAGAACGACACC | 1252-1273 |
| AD-68292.1 | 623 | AUCCUGGAAAUAUAUUAACUA | 740 | UAGUUAAUAUAUUUCCAGGAUGA | 1350-1371_G21A |
| AD-68298.1 | 624 | GCAUUUUGAGAGGUGAUGAUA | 741 | UAUCAUCACCUCUCAAAAUGCCC | 734-755_G21A |
| AD-68277.1 | 625 | CAGGGGGAGAAAGGUGUUCAA | 742 | UUGAACACCUUUCUCCCCCUGGA | 994-1014 |
| AD-68289.1 | 626 | GGAAAUAUAUUAACUGUUAAA | 743 | UUUAACAGUUAAUAUAUUUCCAG | 1355-1376 |
| AD-68272.1 | 627 | CAUUGGUGAGGAAAAAUCCUU | 744 | AAGGAUUUUUCCUCACCAAUGUC | 1097-1117 |
| AD-68282.1 | 628 | GGGAGAAAGGUGUUCAAGAUA | 745 | UAUCUUGAACACCUUUCUCCCCC | 998-1018_G21A |
| AD-68285.1 | 629 | GGCAUUUUGAGAGGUGAUGAU | 746 | AUCAUCACCUCUCAAAAUGCCCU | 733-754 |
| AD-68290.1 | 630 | UACAAAGGGUGUCGUUCUUUU | 747 | AAAAGAACGACACCCUUUGUAUU | 1243-1264 |
| AD-68296.1 | 631 | UGGGAUCUUGGUGUCGAAUCA | 748 | UGAUUCGACACCAAGAUCCCAUU | 783-804 |
| AD-68288.1 | 632 | CUGACAGUGCACAAUAUUUUA | 749 | UAAAAUAUUGUGCACUGUCAGAU | 1134-1155_C21A |
| AD-68299.1 | 633 | CAGUGCACAAUAUUUUCCCAU | 750 | AUGGGAAAAUAUUGUGCACUGUC | 1138-1159 |
| AD-68275.1 | 634 | ACUUUUCAAUGGGUGUCCUAA | 751 | UUAGGACACCCAUUGAAAAGUCA | 1302-1322_G21A |
| AD-68274.1 | 635 | ACAUUGGUGAGGAAAAAUCCU | 752 | AGGAUUUUUCCUCACCAAUGUCU | 1096-1116 |
| AD-68294.1 | 636 | UUGCUUUUGACUUUUCAAUGA | 753 | UCAUUGAAAAGUCAAAAGCAAUG | 1293-1314_G21A |
| AD-68302.1 | 637 | CAUUUUGAGAGGUGAUGAUGA | 754 | UCAUCAUCACCUCUCAAAAUGCC | 735-756_C21A |
| AD-68279.1 | 638 | UUGACUUUUCAAUGGGUGUCA | 755 | UGACACCCAUUGAAAAGUCAAAA | 1299-1319_C21A |
| AD-68304.1 | 639 | CGACUUCUGUUUUAGGACAGA | 756 | UCUGUCCUAAAACAGAAGUCGAC | 212-233 |
| AD-68286.1 | 640 | CUCUGAGUGGGUGCCAGAAUA | 757 | UAUUCUGGCACCCACUCAGAGCC | 1058-1079_G21A |
| AD-68291.1 | 641 | GGGUGCCAGAAUGUGAAAGUA | 758 | UACUUUCACAUUCUGGCACCCAC | 1066-1087_C21A |
| AD-68283.1 | 642 | UCAAUGGGUGUCCUAGGAACA | 759 | UGUUCCUAGGACACCCAUUGAAA | 1307-1327_C21A |
| AD-68280.1 | 643 | AAAGUCAUCGACAAGACAUUA | 760 | UAAUGUCUUGUCGAUGACUUUCA | 1081-1101_G21A |
| AD-68293.1 | 644 | AUUUUGAGAGGUGAUGAUGCA | 761 | UGCAUCAUCACCUCUCAAAAUGC | 736-757_C21A |
| AD-68276.1 | 645 | AUCGACAAGACAUUGGUGAGA | 762 | UCUCACCAAUGUCUUGUCGAUGA | 1087-1107_G21A |
| AD-68308.1 | 646 | GGUGCCAGAAUGUGAAAGUCA | 763 | UGACUUUCACAUUCUGGCACCCA | 1067-1088 |
| AD-68278.1 | 647 | GACAGUGCACAAUAUUUUCCA | 764 | UGGAAAAUAUUGUGCACUGUCAG | 1136-1156_C21A |
| AD-68307.1 | 648 | ACAAAGAGACACUGUGCAGAA | 765 | UUCUGCACAGUGUCUCUUUGUCA | 1191-1212_G21A |
| AD-68284.1 | 649 | UUUUCAAUGGGUGUCCUAGGA | 766 | UCCUAGGACACCCAUUGAAAAGU | 1304-1324 |
| AD-68301.1 | 650 | CCGUUUCCAAGAUCUGACAGU | 767 | ACUGUCAGAUCUUGGAAACGGCC | 1121-1142 |
| AD-68281.1 | 651 | AGGGGGAGAAAGGUGUUCAAA | 768 | UUUGAACACCUUUCUCCCCCUGG | 995-1015_G21A |
| AD-68305.1 | 652 | AGUCAUCGACAAGACAUUGGU | 769 | ACCAAUGUCUUGUCGAUGACUUU | 1083-1104 |

TABLE 8
Additional Human/Mouse/Cyno HAO1 Modified and Unmodified Sense Strand iRNA Sequences

| | | Unmodified sense strand sequence | |
|---|---|---|---|
| Duplex Name | Modified sense strand sequence 5′ to 3′ | 5′ to 3′ | SEQ ID NO: |
| AD-40257.1 | uucAAuGGGuGuccuAGGAdTsdT | UUCAAUGGGUGUCCUAGGA | 770 & 771 |
| AD-40257.2 | uucAAuGGGuGuccuAGGAdTsdT | UUCAAUGGGUGUCCUAGGA | 770 & 771 |
| AD-63102.1 | AcAAcuGGAGGGAcAucGudTsdT | ACAACUGGAGGGACAUCGU | 772 & 773 |
| AD-63102.2 | AcAAcuGGAGGGAcAucGudTsdT | ACAACUGGAGGGACAUCGU | 772 & 773 |
| AD-63102.3 | AcAAcuGGAGGGAcAucGudTsdT | ACAACUGGAGGGACAUCGU | 772 & 773 |

TABLE 9
Additional Human/Mouse/Cyno HAO1 Modified and Unmodified Antisense Strand iRNA Sequences

| | Modified antisense strand sequence 5′ | Unmodified antisense strand | |
|---|---|---|---|
| Duplex Name | to 3′ | sequence 5′ to 3′ | SEQ ID NO: |
| AD-40257.1 | UCCuAGGAcACCcAUUGAAdTsdT | UCCUAGGACACCCAUUGAA | 774 & 775 |
| AD-40257.2 | UCCuAGGAcACCcAUUGAAdTsdT | UCCUAGGACACCCAUUGAA | 774 & 775 |
| AD-63102.1 | ACGAUGUCCCUCcAGUUGUdTsdT | ACGAUGUCCCUCCAGUUGU | 776 & 777 |
| AD-63102.2 | ACGAUGUCCCUCcAGUUGUdTsdT | ACGAUGUCCCUCCAGUUGU | 776 & 777 |
| AD-63102.3 | ACGAUGUCCCUCcAGUUGUdTsdT | ACGAUGUCCCUCCAGUUGU | 776 & 777 |

TABLE 10
Additional Human/Cyno/Mouse/Rat and Human/Cyno/Rat HAO1 Modified Sense Strand iRNA Sequences

| Duplex Name | Modified sense strand sequence | SEQ ID NO: |
|---|---|---|
| AD-62989.2 | UfscsCfuAfgGfaAfCfCfuUfuUfaGfaAfaUfL96 | 778 |
| AD-62994.2 | GfsasCfuUfuCfaUfCfCfuGfgAfaAfuAfuAfL96 | 779 |
| AD-62933.2 | GfsasAfuGfuGfaAfAfGfuCfaUfcGfaCfaAfL96 | 780 |
| AD-62935.2 | CfsasUfuGfgUfgAfGfGfaAfaAfaUfcCfuUfL96 | 781 |
| AD-62940.2 | AfsusCfgAfcAfaGfAfCfaUfuGfgUfgAfgAfL96 | 782 |
| AD-62941.2 | AfscsAfuUfgGfuGfAfGfgAfaAfaAfuCfcUfL96 | 783 |
| AD-62944.2 | GfsasAfaGfuCfaUfCfGfaCfaAfgAfcAfuUfL96 | 784 |
| AD-62965.2 | AfsasAfgUfcAfuCfGfAfcAfaGfaCfaUfuAfL96 | 785 |

TABLE 11
Additional Human/Cyno/Mouse/Rat and Human/Cyno/Rat HA01 Modified Antisense Strand iRNA Sequences

| Duplex Name | Modified antisense strand | SEQ ID NO: |
|---|---|---|
| AD-62989.2 | asUfsuUfcUfaAfaAfgguUfcCfuAfgGfascsa | 786 |
| AD-62994.2 | usAfsuAfuUfuCfcAfggaUfgAfaAfgUfcscsa | 787 |
| AD-62933.2 | usUfsgUfcGfaUfgAfcuuUfcAfcAfuUfcsusg | 788 |
| AD-62935.2 | asAfsgGfaUfuUfuUfccuCfaCfcAfaUfgsusc | 789 |
| AD-62940.2 | usCfsuCfaCfcAfaUfgucUfuGfuCfgAfusgsa | 790 |
| AD-62941.2 | asGfsgAfuUfuUfuCfcucAfcCfaAfuGfuscsu | 791 |
| AD-62944.2 | asAfsuGfuCfuUfgUfcgaUfgAfcUfuUfcsasc | 792 |
| AD-62965.2 | usAfsaUfgUfcUfuGfucgAfuGfaCfuUfuscsa | 793 |

TABLE 12
Additional Human Unmodified and Modifieded Sense and Antisense Strand HAO1 iRNA Sequences Targeting
NM_017545.2

| | SEQ ID | | SEQ ID | | |
|---|---|---|---|---|---|
| Unmodified sequence 5′ to 3′ | NO: | Modified sequence 5′ to 3′ | NO: | Strand | Length |
| AUGUAUGUUACUUCUUAGAGA | 794 | asusguauGfuUfAfCfuucuuagagaL96 | 1890 | sense | 21 |
| UCUCUAAGAAGUAACAUACAUCC | 795 | usCfsucuAfaGfAfaguaAfcAfuacauscsc | 1891 | antisense | 23 |
| UGUAUGUUACUUCUUAGAGAG | 796 | usgsuaugUfuAfCfUfucuuagagagL96 | 1892 | sense | 21 |
| CUCUCUAAGAAGUAACAUACAUC | 797 | csUfscucUfaAfGfaaguAfaCfauacasusc | 1893 | antisense | 23 |
| UAGGAUGUAUGUUACUUCUUA | 798 | usasggauGfuAfUfGfuuacuucuuaL96 | 1894 | sense | 21 |
| UAAGAAGUAACAUACAUCCUAAA | 799 | usAfsagaAfgUfAfacauAfcAfuccuasasa | 1895 | antisense | 23 |
| UUAGGAUGUAUGUUACUUCUU | 800 | ususaggaUfgUfAfUfguuacuucuuL96 | 1896 | sense | 21 |
| AAGAAGUAACAUACAUCCUAAAA | 801 | asAfsgaaGfuAfAfcauaCfaUfccuaasasa | 1897 | antisense | 23 |
| AGAAAGGUGUUCAAGAUGUCC | 802 | asgsaaagGfuGfUfUfcaagauguccL96 | 1898 | sense | 21 |
| GGACAUCUUGAACACCUUUCUCC | 803 | gsGfsacaUfcUfUfgaacAfcCfuuucuscsc | 1899 | antisense | 23 |
| GAAAGGUGUUCAAGAUGUCCU | 804 | gsasaaggUfgUfUfCfaagauguccuL96 | 1900 | sense | 21 |
| AGGACAUCUUGAACACCUUUCUC | 805 | asGfsgacAfuCfUfugaaCfaCfcuuucsusc | 1901 | antisense | 23 |
| GGGGAGAAAGGUGUUCAAGAU | 806 | gsgsggagAfaAfGfGfuguucaagauL96 | 1902 | sense | 21 |
| AUCUUGAACACCUUUCUCCCCCU | 807 | asUfscuuGfaAfCfaccuUfuCfuccccscsu | 1903 | antisense | 23 |
| GGGGGAGAAAGGUGUUCAAGA | 808 | gsgsgggaGfaAfAfGfguguucaagaL96 | 1904 | sense | 21 |
| UCUUGAACACCUUUCUCCCCCUG | 809 | usCfsuugAfaCfAfccuuUfcUfcccccsusg | 1905 | antisense | 23 |
| AGAAACUUUGGCUGAUAAUAU | 810 | asgsaaacUfuUfGfGfcugauaauauL96 | 1906 | sense | 21 |
| AUAUUAUCAGCCAAAGUUUCUUC | 811 | asUfsauuAfuCfAfgccaAfaGfuuucususc | 1907 | antisense | 23 |
| GAAACUUUGGCUGAUAAUAUU | 812 | gsasaacuUfuGfGfCfugauaauauuL96 | 1908 | sense | 21 |
| AAUAUUAUCAGCCAAAGUUUCUU | 813 | asAfsuauUfaUfCfagccAfaAfguuucsusu | 1909 | antisense | 23 |
| AUGAAGAAACUUUGGCUGAUA | 814 | asusgaagAfaAfCfUfuuggcugauaL96 | 1910 | sense | 21 |
| UAUCAGCCAAAGUUUCUUCAUCA | 815 | usAfsucaGfcCfAfaaguUfuCfuucauscsa | 1911 | antisense | 23 |
| GAUGAAGAAACUUUGGCUGAU | 816 | gsasugaaGfaAfAfCfuuuggcugauL96 | 1912 | sense | 21 |
| AUCAGCCAAAGUUUCUUCAUCAU | 817 | asUfscagCfcAfAfaguuUfcUfucaucsasu | 1913 | antisense | 23 |
| AAGGCACUGAUGUUCUGAAAG | 818 | asasggcaCfuGfAfUfguucugaaagL96 | 1914 | sense | 21 |
| CUUUCAGAACAUCAGUGCCUUUC | 819 | csUfsuucAfgAfAfcaucAfgUfgccuususc | 1915 | antisense | 23 |
| AGGCACUGAUGUUCUGAAAGC | 820 | asgsgcacUfgAfUfGfuucugaaagcL96 | 1916 | sense | 21 |
| GCUUUCAGAACAUCAGUGCCUUU | 821 | gsCfsuuuCfaGfAfacauCfaGfugccususu | 1917 | antisense | 23 |
| CGGAAAGGCACUGAUGUUCUG | 822 | csgsgaaaGfgCfAfCfugauguucugL96 | 1918 | sense | 21 |
| CAGAACAUCAGUGCCUUUCCGCA | 823 | csAfsgaaCfaUfCfagugCfcUfuuccgscsa | 1919 | antisense | 23 |
| GCGGAAAGGCACUGAUGUUCU | 824 | gscsggaaAfgGfCfAfcugauguucuL96 | 1920 | sense | 21 |
| AGAACAUCAGUGCCUUUCCGCAC | 825 | asGfsaacAfuCfAfgugcCfuUfuccgcsasc | 1921 | antisense | 23 |
| AGAAGACUGACAUCAUUGCCA | 826 | asgsaagaCfuGfAfCfaucauugccaL96 | 1922 | sense | 21 |
| UGGCAAUGAUGUCAGUCUUCUCA | 827 | usGfsgcaAfuGfAfugucAfgUfcuucuscsa | 1923 | antisense | 23 |
| GAAGACUGACAUCAUUGCCAA | 828 | gsasagacUfgAfCfAfucauugccaaL96 | 1924 | sense | 21 |
| UUGGCAAUGAUGUCAGUCUUCUC | 829 | usUfsggcAfaUfGfauguCfaGfucuucsusc | 1925 | antisense | 23 |
| GCUGAGAAGACUGACAUCAUU | 830 | gscsugagAfaGfAfCfugacaucauuL96 | 1926 | sense | 21 |
| AAUGAUGUCAGUCUUCUCAGCCA | 831 | asAfsugaUfgUfCfagucUfuCfucagcscsa | 1927 | antisense | 23 |
| GGCUGAGAAGACUGACAUCAU | 832 | gsgscugaGfaAfGfAfcugacaucauL96 | 1928 | sense | 21 |
| AUGAUGUCAGUCUUCUCAGCCAU | 833 | asUfsgauGfuCfAfgucuUfcUfcagccsasu | 1929 | antisense | 23 |
| UAAUGCCUGAUUCACAACUUU | 834 | usasaugcCfuGfAfUfucacaacuuuL96 | 1930 | sense | 21 |
| AAAGUUGUGAAUCAGGCAUUACC | 835 | asAfsaguUfgUfGfaaucAfgGfcauuascsc | 1931 | antisense | 23 |
| AAUGCCUGAUUCACAACUUUG | 836 | asasugccUfgAfUfUfcacaacuuugL96 | 1932 | sense | 21 |
| CAAAGUUGUGAAUCAGGCAUUAC | 837 | csAfsaagUfuGfUfgaauCfaGfgcauusasc | 1933 | antisense | 23 |
| UUGGUAAUGCCUGAUUCACAA | 838 | ususgguaAfuGfCfCfugauucacaaL96 | 1934 | sense | 21 |
| UUGUGAAUCAGGCAUUACCAACA | 839 | usUfsgugAfaUfCfaggcAfuUfaccaascsa | 1935 | antisense | 23 |
| GUUGGUAAUGCCUGAUUCACA | 840 | gsusugguAfaUfGfCfcugauucacaL96 | 1936 | sense | 21 |
| UGUGAAUCAGGCAUUACCAACAC | 841 | usGfsugaAfuCfAfggcaUfuAfccaacsasc | 1937 | antisense | 23 |
| UAUCAAAUGGCUGAGAAGACU | 842 | usasucaaAfuGfGfCfugagaagacuL96 | 1938 | sense | 21 |
| AGUCUUCUCAGCCAUUUGAUAUC | 843 | asGfsucuUfcUfCfagccAfuUfugauasusc | 1939 | antisense | 23 |
| AUCAAAUGGCUGAGAAGACUG | 844 | asuscaaaUfgGfCfUfgagaagacugL96 | 1940 | sense | 21 |
| CAGUCUUCUCAGCCAUUUGAUAU | 845 | csAfsgucUfuCfUfcagcCfaUfuugausasu | 1941 | antisense | 23 |
| AAGAUAUCAAAUGGCUGAGAA | 846 | asasgauaUfcAfAfAfuggcugagaaL96 | 1942 | sense | 21 |
| UUCUCAGCCAUUUGAUAUCUUCC | 847 | usUfscucAfgCfCfauuuGfaUfaucuuscsc | 1943 | antisense | 23 |
| GAAGAUAUCAAAUGGCUGAGA | 848 | gsasagauAfuCfAfAfauggcugagaL96 | 1944 | sense | 21 |
| UCUCAGCCAUUUGAUAUCUUCCC | 849 | usCfsucaGfcCfAfuuugAfuAfucuucscsc | 1945 | antisense | 23 |
| UCUGACAGUGCACAAUAUUUU | 850 | uscsugacAfgUfGfCfacaauauuuuL96 | 1946 | sense | 21 |
| AAAAUAUUGUGCACUGUCAGAUC | 851 | asAfsaauAfuUfGfugcaCfuGfucagasusc | 1947 | antisense | 23 |
| CUGACAGUGCACAAUAUUUUC | 852 | csusgacaGfuGfCfAfcaauauuuucL96 | 1948 | sense | 21 |
| GAAAAUAUUGUGCACUGUCAGAU | 853 | gsAfsaaaUfaUfUfgugcAfcUfgucagsasu | 1949 | antisense | 23 |
| AAGAUCUGACAGUGCACAAUA | 854 | asasgaucUfgAfCfAfgugcacaauaL96 | 1950 | sense | 21 |
| UAUUGUGCACUGUCAGAUCUUGG | 855 | usAfsuugUfgCfAfcuguCfaGfaucuusgsg | 1951 | antisense | 23 |
| CAAGAUCUGACAGUGCACAAU | 856 | csasagauCfuGfAfCfagugcacaauL96 | 1952 | sense | 21 |
| AUUGUGCACUGUCAGAUCUUGGA | 857 | asUfsuguGfcAfCfugucAfgAfucuugsgsa | 1953 | antisense | 23 |
| ACUGAUGUUCUGAAAGCUCUG | 858 | ascsugauGfuUfCfUfgaaagcucugL96 | 1954 | sense | 21 |
| CAGAGCUUUCAGAACAUCAGUGC | 859 | csAfsgagCfuUfUfcagaAfcAfucagusgsc | 1955 | antisense | 23 |
| CUGAUGUUCUGAAAGCUCUGG | 860 | csusgaugUfuCfUfGfaaagcucuggL96 | 1956 | sense | 21 |
| CCAGAGCUUUCAGAACAUCAGUG | 861 | csCfsagaGfcUfUfucagAfaCfaucagsusg | 1957 | antisense | 23 |
| AGGCACUGAUGUUCUGAAAGC | 862 | asgsgcacUfgAfUfGfuucugaaagcL96 | 1958 | sense | 21 |
| GCUUUCAGAACAUCAGUGCCUUU | 863 | gsCfsuuuCfaGfAfacauCfaGfugccususu | 1959 | antisense | 23 |
| AAGGCACUGAUGUUCUGAAAG | 864 | asasggcaCfuGfAfUfguucugaaagL96 | 1960 | sense | 21 |
| CUUUCAGAACAUCAGUGCCUUUC | 865 | csUfsuucAfgAfAfcaucAfgUfgccuususc | 1961 | antisense | 23 |
| AACAACAUGCUAAAUCAGUAC | 866 | asascaacAfuGfCfUfaaaucaguacL96 | 1962 | sense | 21 |
| GUACUGAUUUAGCAUGUUGUUCA | 867 | gsUfsacuGfaUfUfuagcAfuGfuuguuscsa | 1963 | antisense | 23 |
| ACAACAUGCUAAAUCAGUACU | 868 | ascsaacaUfgCfUfAfaaucaguacuL96 | 1964 | sense | 21 |
| AGUACUGAUUUAGCAUGUUGUUC | 869 | asGfsuacUfgAfUfuuagCfaUfguugususc | 1965 | antisense | 23 |
| UAUGAACAACAUGCUAAAUCA | 870 | usasugaaCfaAfCfAfugcuaaaucaL96 | 1966 | sense | 21 |
| UGAUUUAGCAUGUUGUUCAUAAU | 871 | usGfsauuUfaGfCfauguUfgUfucauasasu | 1967 | antisense | 23 |
| UUAUGAACAACAUGCUAAAUC | 872 | ususaugaAfcAfAfCfaugcuaaaucL96 | 1968 | sense | 21 |
| GAUUUAGCAUGUUGUUCAUAAUC | 873 | gsAfsuuuAfgCfAfuguuGfuUfcauaasusc | 1969 | antisense | 23 |
| UCUUUAGUGUCUGAAUAUAUC | 874 | uscsuuuaGfuGfUfCfugaauauaucL96 | 1970 | sense | 21 |
| GAUAUAUUCAGACACUAAAGAUG | 875 | gsAfsuauAfuUfCfagacAfcUfaaagasusg | 1971 | antisense | 23 |
| CUUUAGUGUCUGAAUAUAUCC | 876 | csusuuagUfgUfCfUfgaauauauccL96 | 1972 | sense | 21 |
| GGAUAUAUUCAGACACUAAAGAU | 877 | gsGfsauaUfaUfUfcagaCfaCfuaaagsasu | 1973 | antisense | 23 |
| CACAUCUUUAGUGUCUGAAUA | 878 | csascaucUfuUfAfGfugucugaauaL96 | 1974 | sense | 21 |
| UAUUCAGACACUAAAGAUGUGAU | 879 | usAfsuucAfgAfCfacuaAfaGfaugugsasu | 1975 | antisense | 23 |
| UCACAUCUUUAGUGUCUGAAU | 880 | uscsacauCfuUfUfAfgugucugaauL96 | 1976 | sense | 21 |
| AUUCAGACACUAAAGAUGUGAUU | 881 | asUfsucaGfaCfAfcuaaAfgAfugugasusu | 1977 | antisense | 23 |
| UGAUACUUCUUUGAAUGUAGA | 882 | usgsauacUfuCfUfUfugaauguagaL96 | 1978 | sense | 21 |
| UCUACAUUCAAAGAAGUAUCACC | 883 | usCfsuacAfuUfCfaaagAfaGfuaucascsc | 1979 | antisense | 23 |
| GAUACUUCUUUGAAUGUAGAU | 884 | gsasuacuUfcUfUfUfgaauguagauL96 | 1980 | sense | 21 |
| AUCUACAUUCAAAGAAGUAUCAC | 885 | asUfscuaCfaUfUfcaaaGfaAfguaucsasc | 1981 | antisense | 23 |
| UUGGUGAUACUUCUUUGAAUG | 886 | ususggugAfuAfCfUfucuuugaaugL96 | 1982 | sense | 21 |
| CAUUCAAAGAAGUAUCACCAAUU | 887 | csAfsuucAfaAfGfaaguAfuCfaccaasusu | 1983 | antisense | 23 |
| AUUGGUGAUACUUCUUUGAAU | 888 | asusugguGfaUfAfCfuucuuugaauL96 | 1984 | sense | 21 |
| AUUCAAAGAAGUAUCACCAAUUA | 889 | asUfsucaAfaGfAfaguaUfcAfccaaususa | 1985 | antisense | 23 |
| AAUAACCUGUGAAAAUGCUCC | 890 | asasuaacCfuGfUfGfaaaaugcuccL96 | 1986 | sense | 21 |
| GGAGCAUUUUCACAGGUUAUUGC | 891 | gsGfsagcAfuUfUfucacAfgGfuuauusgsc | 1987 | antisense | 23 |
| AUAACCUGUGAAAAUGCUCCC | 892 | asusaaccUfgUfGfAfaaaugcucccL96 | 1988 | sense | 21 |
| GGGAGCAUUUUCACAGGUUAUUG | 893 | gsGfsgagCfaUfUfuucaCfaGfguuaususg | 1989 | antisense | 23 |
| UAGCAAUAACCUGUGAAAAUG | 894 | usasgcaaUfaAfCfCfugugaaaaugL96 | 1990 | sense | 21 |
| CAUUUUCACAGGUUAUUGCUAUC | 895 | csAfsuuuUfcAfCfagguUfaUfugcuasusc | 1991 | antisense | 23 |
| AUAGCAAUAACCUGUGAAAAU | 896 | asusagcaAfuAfAfCfcugugaaaauL96 | 1992 | sense | 21 |
| AUUUUCACAGGUUAUUGCUAUCC | 897 | asUfsuuuCfaCfAfgguuAfuUfgcuauscsc | 1993 | antisense | 23 |
| AAUCACAUCUUUAGUGUCUGA | 898 | asasucacAfuCfUfUfuagugucugaL96 | 1994 | sense | 21 |
| UCAGACACUAAAGAUGUGAUUGG | 899 | usCfsagaCfaCfUfaaagAfuGfugauusgsg | 1995 | antisense | 23 |
| AUCACAUCUUUAGUGUCUGAA | 900 | asuscacaUfcUfUfUfagugucugaaL96 | 1996 | sense | 21 |
| UUCAGACACUAAAGAUGUGAUUG | 901 | usUfscagAfcAfCfuaaaGfaUfgugaususg | 1997 | antisense | 23 |
| UUCCAAUCACAUCUUUAGUGU | 902 | ususccaaUfcAfCfAfucuuuaguguL96 | 1998 | sense | 21 |
| ACACUAAAGAUGUGAUUGGAAAU | 903 | asCfsacuAfaAfGfauguGfaUfuggaasasu | 1999 | antisense | 23 |
| UUUCCAAUCACAUCUUUAGUG | 904 | ususuccaAfuCfAfCfaucuuuagugL96 | 2000 | sense | 21 |
| CACUAAAGAUGUGAUUGGAAAUC | 905 | csAfscuaAfaGfAfugugAfuUfggaaasusc | 2001 | antisense | 23 |
| ACGGGCAUGAUGUUGAGUUCC | 906 | ascsgggcAfuGfAfUfguugaguuccL96 | 2002 | sense | 21 |
| GGAACUCAACAUCAUGCCCGUUC | 907 | gsGfsaacUfcAfAfcaucAfuGfcccgususc | 2003 | antisense | 23 |
| CGGGCAUGAUGUUGAGUUCCU | 908 | csgsggcaUfgAfUfGfuugaguuccuL96 | 2004 | sense | 21 |
| AGGAACUCAACAUCAUGCCCGUU | 909 | asGfsgaaCfuCfAfacauCfaUfgcccgsusu | 2005 | antisense | 23 |
| GGGAACGGGCAUGAUGUUGAG | 910 | gsgsgaacGfgGfCfAfugauguugagL96 | 2006 | sense | 21 |
| CUCAACAUCAUGCCCGUUCCCAG | 911 | csUfscaaCfaUfCfaugcCfcGfuucccsasg | 2007 | antisense | 23 |
| UGGGAACGGGCAUGAUGUUGA | 912 | usgsggaaCfgGfGfCfaugauguugaL96 | 2008 | sense | 21 |
| UCAACAUCAUGCCCGUUCCCAGG | 913 | usCfsaacAfuCfAfugccCfgUfucccasgsg | 2009 | antisense | 23 |
| ACUAAGGUGAAAAGAUAAUGA | 914 | ascsuaagGfuGfAfAfaagauaaugaL96 | 2010 | sense | 21 |
| UCAUUAUCUUUUCACCUUAGUGU | 915 | usCfsauuAfuCfUfuuucAfcCfuuagusgsu | 2011 | antisense | 23 |
| CUAAGGUGAAAAGAUAAUGAU | 916 | csusaaggUfgAfAfAfagauaaugauL96 | 2012 | sense | 21 |
| AUCAUUAUCUUUUCACCUUAGUG | 917 | asUfscauUfaUfCfuuuuCfaCfcuuagsusg | 2013 | antisense | 23 |
| AAACACUAAGGUGAAAAGAUA | 918 | asasacacUfaAfGfGfugaaaagauaL96 | 2014 | sense | 21 |
| UAUCUUUUCACCUUAGUGUUUGC | 919 | usAfsucuUfuUfCfaccuUfaGfuguuusgsc | 2015 | antisense | 23 |
| CAAACACUAAGGUGAAAAGAU | 920 | csasaacaCfuAfAfGfgugaaaagauL96 | 2016 | sense | 21 |
| AUCUUUUCACCUUAGUGUUUGCU | 921 | asUfscuuUfuCfAfccuuAfgUfguuugscsu | 2017 | antisense | 23 |
| AGGUAGCACUGGAGAGAAUUG | 922 | asgsguagCfaCfUfGfgagagaauugL96 | 2018 | sense | 21 |
| CAAUUCUCUCCAGUGCUACCUUC | 923 | csAfsauuCfuCfUfccagUfgCfuaccususc | 2019 | antisense | 23 |
| GGUAGCACUGGAGAGAAUUGG | 924 | gsgsuagcAfcUfGfGfagagaauuggL96 | 2020 | sense | 21 |
| CCAAUUCUCUCCAGUGCUACCUU | 925 | csCfsaauUfcUfCfuccaGfuGfcuaccsusu | 2021 | antisense | 23 |
| GAGAAGGUAGCACUGGAGAGA | 926 | gsasgaagGfuAfGfCfacuggagagaL96 | 2022 | sense | 21 |
| UCUCUCCAGUGCUACCUUCUCAA | 927 | usCfsucuCfcAfGfugcuAfcCfuucucsasa | 2023 | antisense | 23 |
| UGAGAAGGUAGCACUGGAGAG | 928 | usgsagaaGfgUfAfGfcacuggagagL96 | 2024 | sense | 21 |
| CUCUCCAGUGCUACCUUCUCAAA | 929 | csUfscucCfaGfUfgcuaCfcUfucucasasa | 2025 | antisense | 23 |
| AGUGGACUUGCUGCAUAUGUG | 930 | asgsuggaCfuUfGfCfugcauaugugL96 | 2026 | sense | 21 |
| CACAUAUGCAGCAAGUCCACUGU | 931 | csAfscauAfuGfCfagcaAfgUfccacusgsu | 2027 | antisense | 23 |
| GUGGACUUGCUGCAUAUGUGG | 932 | gsusggacUfuGfCfUfgcauauguggL96 | 2028 | sense | 21 |
| CCACAUAUGCAGCAAGUCCACUG | 933 | csCfsacaUfaUfGfcagcAfaGfuccacsusg | 2029 | antisense | 23 |
| CGACAGUGGACUUGCUGCAUA | 934 | csgsacagUfgGfAfCfuugcugcauaL96 | 2030 | sense | 21 |
| UAUGCAGCAAGUCCACUGUCGUC | 935 | usAfsugcAfgCfAfagucCfaCfugucgsusc | 2031 | antisense | 23 |
| ACGACAGUGGACUUGCUGCAU | 936 | ascsgacaGfuGfGfAfcuugcugcauL96 | 2032 | sense | 21 |
| AUGCAGCAAGUCCACUGUCGUCU | 937 | asUfsgcaGfcAfAfguccAfcUfgucguscsu | 2033 | antisense | 23 |
| AAGGUGUUCAAGAUGUCCUCG | 938 | asasggugUfuCfAfAfgauguccucgL96 | 2034 | sense | 21 |
| CGAGGACAUCUUGAACACCUUUC | 939 | csGfsaggAfcAfUfcuugAfaCfaccuususc | 2035 | antisense | 23 |
| AGGUGUUCAAGAUGUCCUCGA | 940 | asgsguguUfcAfAfGfauguccucgaL96 | 2036 | sense | 21 |
| UCGAGGACAUCUUGAACACCUUU | 941 | usCfsgagGfaCfAfucuuGfaAfcaccususu | 2037 | antisense | 23 |
| GAGAAAGGUGUUCAAGAUGUC | 942 | gsasgaaaGfgUfGfUfucaagaugucL96 | 2038 | sense | 21 |
| GACAUCUUGAACACCUUUCUCCC | 943 | gsAfscauCfuUfGfaacaCfcUfuucucscsc | 2039 | antisense | 23 |
| GGAGAAAGGUGUUCAAGAUGU | 944 | gsgsagaaAfgGfUfGfuucaagauguL96 | 2040 | sense | 21 |
| ACAUCUUGAACACCUUUCUCCCC | 945 | asCfsaucUfuGfAfacacCfuUfucuccscsc | 2041 | antisense | 23 |
| AACCGUCUGGAUGAUGUGCGU | 946 | asasccguCfuGfGfAfugaugugcguL96 | 2042 | sense | 21 |
| ACGCACAUCAUCCAGACGGUUGC | 947 | asCfsgcaCfaUfCfauccAfgAfcgguusgsc | 2043 | antisense | 23 |
| ACCGUCUGGAUGAUGUGCGUA | 948 | ascscgucUfgGfAfUfgaugugcguaL96 | 2044 | sense | 21 |
| UACGCACAUCAUCCAGACGGUUG | 949 | usAfscgcAfcAfUfcaucCfaGfacggususg | 2045 | antisense | 23 |
| GGGCAACCGUCUGGAUGAUGU | 950 | gsgsgcaaCfcGfUfCfuggaugauguL96 | 2046 | sense | 21 |
| ACAUCAUCCAGACGGUUGCCCAG | 951 | asCfsaucAfuCfCfagacGfgUfugcccsasg | 2047 | antisense | 23 |
| UGGGCAACCGUCUGGAUGAUG | 952 | usgsggcaAfcCfGfUfcuggaugaugL96 | 2048 | sense | 21 |
| CAUCAUCCAGACGGUUGCCCAGG | 953 | csAfsucaUfcCfAfgacgGfuUfgcccasgsg | 2049 | antisense | 23 |
| GAAACUUUGGCUGAUAAUAUU | 954 | gsasaacuUfuGfGfCfugauaauauuL96 | 2050 | sense | 21 |
| AAUAUUAUCAGCCAAAGUUUCUU | 955 | asAfsuauUfaUfCfagccAfaAfguuucsusu | 2051 | antisense | 23 |
| AAACUUUGGCUGAUAAUAUUG | 956 | asasacuuUfgGfCfUfgauaauauugL96 | 2052 | sense | 21 |
| CAAUAUUAUCAGCCAAAGUUUCU | 957 | csAfsauaUfuAfUfcagcCfaAfaguuuscsu | 2053 | antisense | 23 |
| UGAAGAAACUUUGGCUGAUAA | 958 | usgsaagaAfaCfUfUfuggcugauaaL96 | 2054 | sense | 21 |
| UUAUCAGCCAAAGUUUCUUCAUC | 959 | usUfsaucAfgCfCfaaagUfuUfcuucasusc | 2055 | antisense | 23 |
| AUGAAGAAACUUUGGCUGAUA | 960 | asusgaagAfaAfCfUfuuggcugauaL96 | 2056 | sense | 21 |
| UAUCAGCCAAAGUUUCUUCAUCA | 961 | usAfsucaGfcCfAfaaguUfuCfuucauscsa | 2057 | antisense | 23 |
| AAAGGUGUUCAAGAUGUCCUC | 962 | asasagguGfuUfCfAfagauguccucL96 | 2058 | sense | 21 |
| GAGGACAUCUUGAACACCUUUCU | 963 | gsAfsggaCfaUfCfuugaAfcAfccuuuscsu | 2059 | antisense | 23 |
| AAGGUGUUCAAGAUGUCCUCG | 964 | asasggugUfuCfAfAfgauguccucgL96 | 2060 | sense | 21 |
| CGAGGACAUCUUGAACACCUUUC | 965 | csGfsaggAfcAfUfcuugAfaCfaccuususc | 2061 | antisense | 23 |
| GGAGAAAGGUGUUCAAGAUGU | 966 | gsgsagaaAfgGfUfGfuucaagauguL96 | 2062 | sense | 21 |
| ACAUCUUGAACACCUUUCUCCCC | 967 | asCfsaucUfuGfAfacacCfuUfucuccscsc | 2063 | antisense | 23 |
| GGGAGAAAGGUGUUCAAGAUG | 968 | gsgsgagaAfaGfGfUfguucaagaugL96 | 2064 | sense | 21 |
| CAUCUUGAACACCUUUCUCCCCC | 969 | csAfsucuUfgAfAfcaccUfuUfcucccscsc | 2065 | antisense | 23 |
| AAAUCAGUACUUCCAAAGUCU | 970 | asasaucaGfuAfCfUfuccaaagucuL96 | 2066 | sense | 21 |
| AGACUUUGGAAGUACUGAUUUAG | 971 | asGfsacuUfuGfGfaaguAfcUfgauuusasg | 2067 | antisense | 23 |
| AAUCAGUACUUCCAAAGUCUA | 972 | asasucagUfaCfUfUfccaaagucuaL96 | 2068 | sense | 21 |
| UAGACUUUGGAAGUACUGAUUUA | 973 | usAfsgacUfuUfGfgaagUfaCfugauususa | 2069 | antisense | 23 |
| UGCUAAAUCAGUACUUCCAAA | 974 | usgscuaaAfuCfAfGfuacuuccaaaL96 | 2070 | sense | 21 |
| UUUGGAAGUACUGAUUUAGCAUG | 975 | usUfsuggAfaGfUfacugAfuUfuagcasusg | 2071 | antisense | 23 |
| AUGCUAAAUCAGUACUUCCAA | 976 | asusgcuaAfaUfCfAfguacuuccaaL96 | 2072 | sense | 21 |
| UUGGAAGUACUGAUUUAGCAUGU | 977 | usUfsggaAfgUfAfcugaUfuUfagcausgsu | 2073 | antisense | 23 |
| ACAUCUUUAGUGUCUGAAUAU | 978 | ascsaucuUfuAfGfUfgucugaauauL96 | 2074 | sense | 21 |
| AUAUUCAGACACUAAAGAUGUGA | 979 | asUfsauuCfaGfAfcacuAfaAfgaugusgsa | 2075 | antisense | 23 |
| CAUCUUUAGUGUCUGAAUAUA | 980 | csasucuuUfaGfUfGfucugaauauaL96 | 2076 | sense | 21 |
| UAUAUUCAGACACUAAAGAUGUG | 981 | usAfsuauUfcAfGfacacUfaAfagaugsusg | 2077 | antisense | 23 |
| AAUCACAUCUUUAGUGUCUGA | 982 | asasucacAfuCfUfUfuagugucugaL96 | 2078 | sense | 21 |
| UCAGACACUAAAGAUGUGAUUGG | 983 | usCfsagaCfaCfUfaaagAfuGfugauusgsg | 2079 | antisense | 23 |
| CAAUCACAUCUUUAGUGUCUG | 984 | csasaucaCfaUfCfUfuuagugucugL96 | 2080 | sense | 21 |
| CAGACACUAAAGAUGUGAUUGGA | 985 | csAfsgacAfcUfAfaagaUfgUfgauugsgsa | 2081 | antisense | 23 |
| GCAUGUAUUACUUGACAAAGA | 986 | gscsauguAfuUfAfCfuugacaaagaL96 | 2082 | sense | 21 |
| UCUUUGUCAAGUAAUACAUGCUG | 987 | usCfsuuuGfuCfAfaguaAfuAfcaugcsusg | 2083 | antisense | 23 |
| CAUGUAUUACUUGACAAAGAG | 988 | csasuguaUfuAfCfUfugacaaagagL96 | 2084 | sense | 21 |
| CUCUUUGUCAAGUAAUACAUGCU | 989 | csUfscuuUfgUfCfaaguAfaUfacaugscsu | 2085 | antisense | 23 |
| UUCAGCAUGUAUUACUUGACA | 990 | ususcagcAfuGfUfAfuuacuugacaL96 | 2086 | sense | 21 |
| UGUCAAGUAAUACAUGCUGAAAA | 991 | usGfsucaAfgUfAfauacAfuGfcugaasasa | 2087 | antisense | 23 |
| UUUCAGCAUGUAUUACUUGAC | 992 | ususucagCfaUfGfUfauuacuugacL96 | 2088 | sense | 21 |
| GUCAAGUAAUACAUGCUGAAAAA | 993 | gsUfscaaGfuAfAfuacaUfgCfugaaasasa | 2089 | antisense | 23 |
| AUGUUACUUCUUAGAGAGAAA | 994 | asusguuaCfuUfCfUfuagagagaaaL96 | 2090 | sense | 21 |
| UUUCUCUCUAAGAAGUAACAUAC | 995 | usUfsucuCfuCfUfaagaAfgUfaacausasc | 2091 | antisense | 23 |
| UGUUACUUCUUAGAGAGAAAU | 996 | usgsuuacUfuCfUfUfagagagaaauL96 | 2092 | sense | 21 |
| AUUUCUCUCUAAGAAGUAACAUA | 997 | asUfsuucUfcUfCfuaagAfaGfuaacasusa | 2093 | antisense | 23 |
| AUGUAUGUUACUUCUUAGAGA | 998 | asusguauGfuUfAfCfuucuuagagaL96 | 2094 | sense | 21 |
| UCUCUAAGAAGUAACAUACAUCC | 999 | usCfsucuAfaGfAfaguaAfcAfuacauscsc | 2095 | antisense | 23 |
| GAUGUAUGUUACUUCUUAGAG | 1000 | gsasuguaUfgUfUfAfcuucuuagagL96 | 2096 | sense | 21 |
| CUCUAAGAAGUAACAUACAUCCU | 1001 | csUfscuaAfgAfAfguaaCfaUfacaucscsu | 2097 | antisense | 23 |
| ACAACUUUGAGAAGGUAGCAC | 1002 | ascsaacuUfuGfAfGfaagguagcacL96 | 2098 | sense | 21 |
| GUGCUACCUUCUCAAAGUUGUGA | 1003 | gsUfsgcuAfcCfUfucucAfaAfguugusgsa | 2099 | antisense | 23 |
| CAACUUUGAGAAGGUAGCACU | 1004 | csasacuuUfgAfGfAfagguagcacuL96 | 2100 | sense | 21 |
| AGUGCUACCUUCUCAAAGUUGUG | 1005 | asGfsugcUfaCfCfuucuCfaAfaguugsusg | 2101 | antisense | 23 |
| AUUCACAACUUUGAGAAGGUA | 1006 | asusucacAfaCfUfUfugagaagguaL96 | 2102 | sense | 21 |
| UACCUUCUCAAAGUUGUGAAUCA | 1007 | usAfsccuUfcUfCfaaagUfuGfugaauscsa | 2103 | antisense | 23 |
| GAUUCACAACUUUGAGAAGGU | 1008 | gsasuucaCfaAfCfUfuugagaagguL96 | 2104 | sense | 21 |
| ACCUUCUCAAAGUUGUGAAUCAG | 1009 | asCfscuuCfuCfAfaaguUfgUfgaaucsasg | 2105 | antisense | 23 |
| AACAUGCUAAAUCAGUACUUC | 1010 | asascaugCfuAfAfAfucaguacuucL96 | 2106 | sense | 21 |
| GAAGUACUGAUUUAGCAUGUUGU | 1011 | gsAfsaguAfcUfGfauuuAfgCfauguusgsu | 2107 | antisense | 23 |
| ACAUGCUAAAUCAGUACUUCC | 1012 | ascsaugcUfaAfAfUfcaguacuuccL96 | 2108 | sense | 21 |
| GGAAGUACUGAUUUAGCAUGUUG | 1013 | gsGfsaagUfaCfUfgauuUfaGfcaugususg | 2109 | antisense | 23 |
| GAACAACAUGCUAAAUCAGUA | 1014 | gsasacaaCfaUfGfCfuaaaucaguaL96 | 2110 | sense | 21 |
| UACUGAUUUAGCAUGUUGUUCAU | 1015 | usAfscugAfuUfUfagcaUfgUfuguucsasu | 2111 | antisense | 23 |
| UGAACAACAUGCUAAAUCAGU | 1016 | usgsaacaAfcAfUfGfcuaaaucaguL96 | 2112 | sense | 21 |
| ACUGAUUUAGCAUGUUGUUCAUA | 1017 | asCfsugaUfuUfAfgcauGfuUfguucasusa | 2113 | antisense | 23 |
| AAACCAGUACUUUAUCAUUUU | 1018 | asasaccaGfuAfCfUfuuaucauuuuL96 | 2114 | sense | 21 |
| AAAAUGAUAAAGUACUGGUUUCA | 1019 | asAfsaauGfaUfAfaaguAfcUfgguuuscsa | 2115 | antisense | 23 |
| AACCAGUACUUUAUCAUUUUC | 1020 | asasccagUfaCfUfUfuaucauuuucL96 | 2116 | sense | 21 |
| GAAAAUGAUAAAGUACUGGUUUC | 1021 | gsAfsaaaUfgAfUfaaagUfaCfugguususc | 2117 | antisense | 23 |
| UUUGAAACCAGUACUUUAUCA | 1022 | ususugaaAfcCfAfGfuacuuuaucaL96 | 2118 | sense | 21 |
| UGAUAAAGUACUGGUUUCAAAAU | 1023 | usGfsauaAfaGfUfacugGfuUfucaaasasu | 2119 | antisense | 23 |
| UUUUGAAACCAGUACUUUAUC | 1024 | ususuugaAfaCfCfAfguacuuuaucL96 | 2120 | sense | 21 |
| GAUAAAGUACUGGUUUCAAAAUU | 1025 | gsAfsuaaAfgUfAfcuggUfuUfcaaaasusu | 2121 | antisense | 23 |
| GAGAAGAUGGGCUACAAGGCC | 1026 | gsasgaagAfuGfGfGfcuacaaggccL96 | 2122 | sense | 21 |
| GGCCUUGUAGCCCAUCUUCUCUG | 1027 | gsGfsccuUfgUfAfgcccAfuCfuucucsusg | 2123 | antisense | 23 |
| AGAAGAUGGGCUACAAGGCCA | 1028 | asgsaagaUfgGfGfCfuacaaggccaL96 | 2124 | sense | 21 |
| UGGCCUUGUAGCCCAUCUUCUCU | 1029 | usGfsgccUfuGfUfagccCfaUfcuucuscsu | 2125 | antisense | 23 |
| GGCAGAGAAGAUGGGCUACAA | 1030 | gsgscagaGfaAfGfAfugggcuacaaL96 | 2126 | sense | 21 |
| UUGUAGCCCAUCUUCUCUGCCUG | 1031 | usUfsguaGfcCfCfaucuUfcUfcugccsusg | 2127 | antisense | 23 |
| AGGCAGAGAAGAUGGGCUACA | 1032 | asgsgcagAfgAfAfGfaugggcuacaL96 | 2128 | sense | 21 |
| UGUAGCCCAUCUUCUCUGCCUGC | 1033 | usGfsuagCfcCfAfucuuCfuCfugccusgsc | 2129 | antisense | 23 |
| AACGGGCAUGAUGUUGAGUUC | 1034 | asascgggCfaUfGfAfuguugaguucL96 | 2130 | sense | 21 |
| GAACUCAACAUCAUGCCCGUUCC | 1035 | gsAfsacuCfaAfCfaucaUfgCfccguuscsc | 2131 | antisense | 23 |
| ACGGGCAUGAUGUUGAGUUCC | 1036 | ascsgggcAfuGfAfUfguugaguuccL96 | 2132 | sense | 21 |
| GGAACUCAACAUCAUGCCCGUUC | 1037 | gsGfsaacUfcAfAfcaucAfuGfcccgususc | 2133 | antisense | 23 |
| UGGGAACGGGCAUGAUGUUGA | 1038 | usgsggaaCfgGfGfCfaugauguugaL96 | 2134 | sense | 21 |
| UCAACAUCAUGCCCGUUCCCAGG | 1039 | usCfsaacAfuCfAfugccCfgUfucccasgsg | 2135 | antisense | 23 |
| CUGGGAACGGGCAUGAUGUUG | 1040 | csusgggaAfcGfGfGfcaugauguugL96 | 2136 | sense | 21 |
| CAACAUCAUGCCCGUUCCCAGGG | 1041 | csAfsacaUfcAfUfgcccGfuUfcccagsgsg | 2137 | antisense | 23 |
| AUGUGGCUAAAGCAAUAGACC | 1042 | asusguggCfuAfAfAfgcaauagaccL96 | 2138 | sense | 21 |
| GGUCUAUUGCUUUAGCCACAUAU | 1043 | gsGfsucuAfuUfGfcuuuAfgCfcacausasu | 2139 | antisense | 23 |
| UGUGGCUAAAGCAAUAGACCC | 1044 | usgsuggcUfaAfAfGfcaauagacccL96 | 2140 | sense | 21 |
| GGGUCUAUUGCUUUAGCCACAUA | 1045 | gsGfsgucUfaUfUfgcuuUfaGfccacasusa | 2141 | antisense | 23 |
| GCAUAUGUGGCUAAAGCAAUA | 1046 | gscsauauGfuGfGfCfuaaagcaauaL96 | 2142 | sense | 21 |
| UAUUGCUUUAGCCACAUAUGCAG | 1047 | usAfsuugCfuUfUfagccAfcAfuaugcsasg | 2143 | antisense | 23 |
| UGCAUAUGUGGCUAAAGCAAU | 1048 | usgscauaUfgUfGfGfcuaaagcaauL96 | 2144 | sense | 21 |
| AUUGCUUUAGCCACAUAUGCAGC | 1049 | asUfsugcUfuUfAfgccaCfaUfaugcasgsc | 2145 | antisense | 23 |
| AGGAUGCUCCGGAAUGUUGCU | 1050 | asgsgaugCfuCfCfGfgaauguugcuL96 | 2146 | sense | 21 |
| AGCAACAUUCCGGAGCAUCCUUG | 1051 | asGfscaaCfaUfUfccggAfgCfauccususg | 2147 | antisense | 23 |
| GGAUGCUCCGGAAUGUUGCUG | 1052 | gsgsaugcUfcCfGfGfaauguugcugL96 | 2148 | sense | 21 |
| CAGCAACAUUCCGGAGCAUCCUU | 1053 | csAfsgcaAfcAfUfuccgGfaGfcauccsusu | 2149 | antisense | 23 |
| UCCAAGGAUGCUCCGGAAUGU | 1054 | uscscaagGfaUfGfCfuccggaauguL96 | 2150 | sense | 21 |
| ACAUUCCGGAGCAUCCUUGGAUA | 1055 | asCfsauuCfcGfGfagcaUfcCfuuggasusa | 2151 | antisense | 23 |
| AUCCAAGGAUGCUCCGGAAUG | 1056 | asusccaaGfgAfUfGfcuccggaaugL96 | 2152 | sense | 21 |
| CAUUCCGGAGCAUCCUUGGAUAC | 1057 | csAfsuucCfgGfAfgcauCfcUfuggausasc | 2153 | antisense | 23 |
| UCACAUCUUUAGUGUCUGAAU | 1058 | uscsacauCfuUfUfAfgugucugaauL96 | 2154 | sense | 21 |
| AUUCAGACACUAAAGAUGUGAUU | 1059 | asUfsucaGfaCfAfcuaaAfgAfugugasusu | 2155 | antisense | 23 |
| CACAUCUUUAGUGUCUGAAUA | 1060 | csascaucUfuUfAfGfugucugaauaL96 | 2156 | sense | 21 |
| UAUUCAGACACUAAAGAUGUGAU | 1061 | usAfsuucAfgAfCfacuaAfaGfaugugsasu | 2157 | antisense | 23 |
| CCAAUCACAUCUUUAGUGUCU | 1062 | cscsaaucAfcAfUfCfuuuagugucuL96 | 2158 | sense | 21 |
| AGACACUAAAGAUGUGAUUGGAA | 1063 | asGfsacaCfuAfAfagauGfuGfauuggsasa | 2159 | antisense | 23 |
| UCCAAUCACAUCUUUAGUGUC | 1064 | uscscaauCfaCfAfUfcuuuagugucL96 | 2160 | sense | 21 |
| GACACUAAAGAUGUGAUUGGAAA | 1065 | gsAfscacUfaAfAfgaugUfgAfuuggasasa | 2161 | antisense | 23 |
| AAAUGUGUUUAGACAACGUCA | 1066 | asasauguGfuUfUfAfgacaacgucaL96 | 2162 | sense | 21 |
| UGACGUUGUCUAAACACAUUUUC | 1067 | usGfsacgUfuGfUfcuaaAfcAfcauuususc | 2163 | antisense | 23 |
| AAUGUGUUUAGACAACGUCAU | 1068 | asasugugUfuUfAfGfacaacgucauL96 | 2164 | sense | 21 |
| AUGACGUUGUCUAAACACAUUUU | 1069 | asUfsgacGfuUfGfucuaAfaCfacauususu | 2165 | antisense | 23 |
| UUGAAAAUGUGUUUAGACAAC | 1070 | ususgaaaAfuGfUfGfuuuagacaacL96 | 2166 | sense | 21 |
| GUUGUCUAAACACAUUUUCAAUG | 1071 | gsUfsuguCfuAfAfacacAfuUfuucaasusg | 2167 | antisense | 23 |
| AUUGAAAAUGUGUUUAGACAA | 1072 | asusugaaAfaUfGfUfguuuagacaaL96 | 2168 | sense | 21 |
| UUGUCUAAACACAUUUUCAAUGU | 1073 | usUfsgucUfaAfAfcacaUfuUfucaausgsu | 2169 | antisense | 23 |
| UACUAAAGGAAGAAUUCCGGU | 1074 | usascuaaAfgGfAfAfgaauuccgguL96 | 2170 | sense | 21 |
| ACCGGAAUUCUUCCUUUAGUAUC | 1075 | asCfscggAfaUfUfcuucCfuUfuaguasusc | 2171 | antisense | 23 |
| ACUAAAGGAAGAAUUCCGGUU | 1076 | ascsuaaaGfgAfAfGfaauuccgguuL96 | 2172 | sense | 21 |
| AACCGGAAUUCUUCCUUUAGUAU | 1077 | asAfsccgGfaAfUfucuuCfcUfuuagusasu | 2173 | antisense | 23 |
| GAGAUACUAAAGGAAGAAUUC | 1078 | gsasgauaCfuAfAfAfggaagaauucL96 | 2174 | sense | 21 |
| GAAUUCUUCCUUUAGUAUCUCGA | 1079 | gsAfsauuCfuUfCfcuuuAfgUfaucucsgsa | 2175 | antisense | 23 |
| CGAGAUACUAAAGGAAGAAUU | 1080 | csgsagauAfcUfAfAfaggaagaauuL96 | 2176 | sense | 21 |
| AAUUCUUCCUUUAGUAUCUCGAG | 1081 | asAfsuucUfuCfCfuuuaGfuAfucucgsasg | 2177 | antisense | 23 |
| AACUUUGGCUGAUAAUAUUGC | 1082 | asascuuuGfgCfUfGfauaauauugcL96 | 2178 | sense | 21 |
| GCAAUAUUAUCAGCCAAAGUUUC | 1083 | gsCfsaauAfuUfAfucagCfcAfaaguususc | 2179 | antisense | 23 |
| ACUUUGGCUGAUAAUAUUGCA | 1084 | ascsuuugGfcUfGfAfuaauauugcaL96 | 2180 | sense | 21 |
| UGCAAUAUUAUCAGCCAAAGUUU | 1085 | usGfscaaUfaUfUfaucaGfcCfaaagususu | 2181 | antisense | 23 |
| AAGAAACUUUGGCUGAUAAUA | 1086 | asasgaaaCfuUfUfGfgcugauaauaL96 | 2182 | sense | 21 |
| UAUUAUCAGCCAAAGUUUCUUCA | 1087 | usAfsuuaUfcAfGfccaaAfgUfuucuuscsa | 2183 | antisense | 23 |
| GAAGAAACUUUGGCUGAUAAU | 1088 | gsasagaaAfcUfUfUfggcugauaauL96 | 2184 | sense | 21 |
| AUUAUCAGCCAAAGUUUCUUCAU | 1089 | asUfsuauCfaGfCfcaaaGfuUfucuucsasu | 2185 | antisense | 23 |
| AAAUGGCUGAGAAGACUGACA | 1090 | asasauggCfuGfAfGfaagacugacaL96 | 2186 | sense | 21 |
| UGUCAGUCUUCUCAGCCAUUUGA | 1091 | usGfsucaGfuCfUfucucAfgCfcauuusgsa | 2187 | antisense | 23 |
| AAUGGCUGAGAAGACUGACAU | 1092 | asasuggcUfgAfGfAfagacugacauL96 | 2188 | sense | 21 |
| AUGUCAGUCUUCUCAGCCAUUUG | 1093 | asUfsgucAfgUfCfuucuCfaGfccauususg | 2189 | antisense | 23 |
| UAUCAAAUGGCUGAGAAGACU | 1094 | usasucaaAfuGfGfCfugagaagacuL96 | 2190 | sense | 21 |
| AGUCUUCUCAGCCAUUUGAUAUC | 1095 | asGfsucuUfcUfCfagccAfuUfugauasusc | 2191 | antisense | 23 |
| AUAUCAAAUGGCUGAGAAGAC | 1096 | asusaucaAfaUfGfGfcugagaagacL96 | 2192 | sense | 21 |
| GUCUUCUCAGCCAUUUGAUAUCU | 1097 | gsUfscuuCfuCfAfgccaUfuUfgauauscsu | 2193 | antisense | 23 |
| GUGGUUCUUAAAUUGUAAGCU | 1098 | gsusgguuCfuUfAfAfauuguaagcuL96 | 2194 | sense | 21 |
| AGCUUACAAUUUAAGAACCACUG | 1099 | asGfscuuAfcAfAfuuuaAfgAfaccacsusg | 2195 | antisense | 23 |
| UGGUUCUUAAAUUGUAAGCUC | 1100 | usgsguucUfuAfAfAfuuguaagcucL96 | 2196 | sense | 21 |
| GAGCUUACAAUUUAAGAACCACU | 1101 | gsAfsgcuUfaCfAfauuuAfaGfaaccascsu | 2197 | antisense | 23 |
| AACAGUGGUUCUUAAAUUGUA | 1102 | asascaguGfgUfUfCfuuaaauuguaL96 | 2198 | sense | 21 |
| UACAAUUUAAGAACCACUGUUUU | 1103 | usAfscaaUfuUfAfagaaCfcAfcuguususu | 2199 | antisense | 23 |
| AAACAGUGGUUCUUAAAUUGU | 1104 | asasacagUfgGfUfUfcuuaaauuguL96 | 2200 | sense | 21 |
| ACAAUUUAAGAACCACUGUUUUA | 1105 | asCfsaauUfuAfAfgaacCfaCfuguuususa | 2201 | antisense | 23 |
| AAGUCAUCGACAAGACAUUGG | 1106 | asasgucaUfcGfAfCfaagacauuggL96 | 2202 | sense | 21 |
| CCAAUGUCUUGUCGAUGACUUUC | 1107 | csCfsaauGfuCfUfugucGfaUfgacuususc | 2203 | antisense | 23 |
| AGUCAUCGACAAGACAUUGGU | 1108 | asgsucauCfgAfCfAfagacauugguL96 | 2204 | sense | 21 |
| ACCAAUGUCUUGUCGAUGACUUU | 1109 | asCfscaaUfgUfCfuuguCfgAfugacususu | 2205 | antisense | 23 |
| GUGAAAGUCAUCGACAAGACA | 1110 | gsusgaaaGfuCfAfUfcgacaagacaL96 | 2206 | sense | 21 |
| UGUCUUGUCGAUGACUUUCACAU | 1111 | usGfsucuUfgUfCfgaugAfcUfuucacsasu | 2207 | antisense | 23 |
| UGUGAAAGUCAUCGACAAGAC | 1112 | usgsugaaAfgUfCfAfucgacaagacL96 | 2208 | sense | 21 |
| GUCUUGUCGAUGACUUUCACAUU | 1113 | gsUfscuuGfuCfGfaugaCfuUfucacasusu | 2209 | antisense | 23 |
| GAUAAUAUUGCAGCAUUUUCC | 1114 | gsasuaauAfuUfGfCfagcauuuuccL96 | 2210 | sense | 21 |
| GGAAAAUGCUGCAAUAUUAUCAG | 1115 | gsGfsaaaAfuGfCfugcaAfuAfuuaucsasg | 2211 | antisense | 23 |
| AUAAUAUUGCAGCAUUUUCCA | 1116 | asusaauaUfuGfCfAfgcauuuuccaL96 | 2212 | sense | 21 |
| UGGAAAAUGCUGCAAUAUUAUCA | 1117 | usGfsgaaAfaUfGfcugcAfaUfauuauscsa | 2213 | antisense | 23 |
| GGCUGAUAAUAUUGCAGCAUU | 1118 | gsgscugaUfaAfUfAfuugcagcauuL96 | 2214 | sense | 21 |
| AAUGCUGCAAUAUUAUCAGCCAA | 1119 | asAfsugcUfgCfAfauauUfaUfcagccsasa | 2215 | antisense | 23 |
| UGGCUGAUAAUAUUGCAGCAU | 1120 | usgsgcugAfuAfAfUfauugcagcauL96 | 2216 | sense | 21 |
| AUGCUGCAAUAUUAUCAGCCAAA | 1121 | asUfsgcuGfcAfAfuauuAfuCfagccasasa | 2217 | antisense | 23 |
| GCUAAUUUGUAUCAAUGAUUA | 1122 | gscsuaauUfuGfUfAfucaaugauuaL96 | 2218 | sense | 21 |
| UAAUCAUUGAUACAAAUUAGCCG | 1123 | usAfsaucAfuUfGfauacAfaAfuuagcscsg | 2219 | antisense | 23 |
| CUAAUUUGUAUCAAUGAUUAU | 1124 | csusaauuUfgUfAfUfcaaugauuauL96 | 2220 | sense | 21 |
| AUAAUCAUUGAUACAAAUUAGCC | 1125 | asUfsaauCfaUfUfgauaCfaAfauuagscsc | 2221 | antisense | 23 |
| CCCGGCUAAUUUGUAUCAAUG | 1126 | cscscggcUfaAfUfUfuguaucaaugL96 | 2222 | sense | 21 |
| CAUUGAUACAAAUUAGCCGGGGG | 1127 | csAfsuugAfuAfCfaaauUfaGfccgggsgsg | 2223 | antisense | 23 |
| CCCCGGCUAAUUUGUAUCAAU | 1128 | cscsccggCfuAfAfUfuuguaucaauL96 | 2224 | sense | 21 |
| AUUGAUACAAAUUAGCCGGGGGA | 1129 | asUfsugaUfaCfAfaauuAfgCfcggggsgsa | 2225 | antisense | 23 |
| UAAUUGGUGAUACUUCUUUGA | 1130 | usasauugGfuGfAfUfacuucuuugaL96 | 2226 | sense | 21 |
| UCAAAGAAGUAUCACCAAUUACC | 1131 | usCfsaaaGfaAfGfuaucAfcCfaauuascsc | 2227 | antisense | 23 |
| AAUUGGUGAUACUUCUUUGAA | 1132 | asasuuggUfgAfUfAfcuucuuugaaL96 | 2228 | sense | 21 |
| UUCAAAGAAGUAUCACCAAUUAC | 1133 | usUfscaaAfgAfAfguauCfaCfcaauusasc | 2229 | antisense | 23 |
| GCGGUAAUUGGUGAUACUUCU | 1134 | gscsgguaAfuUfGfGfugauacuucuL96 | 2230 | sense | 21 |
| AGAAGUAUCACCAAUUACCGCCA | 1135 | asGfsaagUfaUfCfaccaAfuUfaccgcscsa | 2231 | antisense | 23 |
| GGCGGUAAUUGGUGAUACUUC | 1136 | gsgscgguAfaUfUfGfgugauacuucL96 | 2232 | sense | 21 |
| GAAGUAUCACCAAUUACCGCCAC | 1137 | gsAfsaguAfuCfAfccaaUfuAfccgccsasc | 2233 | antisense | 23 |
| CAGUGGUUCUUAAAUUGUAAG | 1138 | csasguggUfuCfUfUfaaauuguaagL96 | 2234 | sense | 21 |
| CUUACAAUUUAAGAACCACUGUU | 1139 | csUfsuacAfaUfUfuaagAfaCfcacugsusu | 2235 | antisense | 23 |
| AGUGGUUCUUAAAUUGUAAGC | 1140 | asgsugguUfcUfUfAfaauuguaagcL96 | 2236 | sense | 21 |
| GCUUACAAUUUAAGAACCACUGU | 1141 | gsCfsuuaCfaAfUfuuaaGfaAfccacusgsu | 2237 | antisense | 23 |
| AAAACAGUGGUUCUUAAAUUG | 1142 | asasaacaGfuGfGfUfucuuaaauugL96 | 2238 | sense | 21 |
| CAAUUUAAGAACCACUGUUUUAA | 1143 | csAfsauuUfaAfGfaaccAfcUfguuuusasa | 2239 | antisense | 23 |
| UAAAACAGUGGUUCUUAAAUU | 1144 | usasaaacAfgUfGfGfuucuuaaauuL96 | 2240 | sense | 21 |
| AAUUUAAGAACCACUGUUUUAAA | 1145 | asAfsuuuAfaGfAfaccaCfuGfuuuuasasa | 2241 | antisense | 23 |
| ACCUGUAUUCUGUUUACAUGU | 1146 | ascscuguAfuUfCfUfguuuacauguL96 | 2242 | sense | 21 |
| ACAUGUAAACAGAAUACAGGUUA | 1147 | asCfsaugUfaAfAfcagaAfuAfcaggususa | 2243 | antisense | 23 |
| CCUGUAUUCUGUUUACAUGUC | 1148 | cscsuguaUfuCfUfGfuuuacaugucL96 | 2244 | sense | 21 |
| GACAUGUAAACAGAAUACAGGUU | 1149 | gsAfscauGfuAfAfacagAfaUfacaggsusu | 2245 | antisense | 23 |
| AUUAACCUGUAUUCUGUUUAC | 1150 | asusuaacCfuGfUfAfuucuguuuacL96 | 2246 | sense | 21 |
| GUAAACAGAAUACAGGUUAAUAA | 1151 | gsUfsaaaCfaGfAfauacAfgGfuuaausasa | 2247 | antisense | 23 |
| UAUUAACCUGUAUUCUGUUUA | 1152 | usasuuaaCfcUfGfUfauucuguuuaL96 | 2248 | sense | 21 |
| UAAACAGAAUACAGGUUAAUAAA | 1153 | usAfsaacAfgAfAfuacaGfgUfuaauasasa | 2249 | antisense | 23 |
| AAGAAACUUUGGCUGAUAAUA | 1154 | asasgaaaCfuUfUfGfgcugauaauaL96 | 2250 | sense | 21 |
| UAUUAUCAGCCAAAGUUUCUUCA | 1155 | usAfsuuaUfcAfGfccaaAfgUfuucuuscsa | 2251 | antisense | 23 |
| AGAAACUUUGGCUGAUAAUAU | 1156 | asgsaaacUfuUfGfGfcugauaauauL96 | 2252 | sense | 21 |
| AUAUUAUCAGCCAAAGUUUCUUC | 1157 | asUfsauuAfuCfAfgccaAfaGfuuucususc | 2253 | antisense | 23 |
| GAUGAAGAAACUUUGGCUGAU | 1158 | gsasugaaGfaAfAfCfuuuggcugauL96 | 2254 | sense | 21 |
| AUCAGCCAAAGUUUCUUCAUCAU | 1159 | asUfscagCfcAfAfaguuUfcUfucaucsasu | 2255 | antisense | 23 |
| UGAUGAAGAAACUUUGGCUGA | 1160 | usgsaugaAfgAfAfAfcuuuggcugaL96 | 2256 | sense | 21 |
| UCAGCCAAAGUUUCUUCAUCAUU | 1161 | usCfsagcCfaAfAfguuuCfuUfcaucasusu | 2257 | antisense | 23 |
| GAAAGGUGUUCAAGAUGUCCU | 1162 | gsasaaggUfgUfUfCfaagauguccuL96 | 2258 | sense | 21 |
| AGGACAUCUUGAACACCUUUCUC | 1163 | asGfsgacAfuCfUfugaaCfaCfcuuucsusc | 2259 | antisense | 23 |
| AAAGGUGUUCAAGAUGUCCUC | 1164 | asasagguGfuUfCfAfagauguccucL96 | 2260 | sense | 21 |
| GAGGACAUCUUGAACACCUUUCU | 1165 | gsAfsggaCfaUfCfuugaAfcAfccuuuscsu | 2261 | antisense | 23 |
| GGGAGAAAGGUGUUCAAGAUG | 1166 | gsgsgagaAfaGfGfUfguucaagaugL96 | 2262 | sense | 21 |
| CAUCUUGAACACCUUUCUCCCCC | 1167 | csAfsucuUfgAfAfcaccUfuUfcucccscsc | 2263 | antisense | 23 |
| GGGGAGAAAGGUGUUCAAGAU | 1168 | gsgsggagAfaAfGfGfuguucaagauL96 | 2264 | sense | 21 |
| AUCUUGAACACCUUUCUCCCCCU | 1169 | asUfscuuGfaAfCfaccuUfuCfuccccscsu | 2265 | antisense | 23 |
| AUCUUGGUGUCGAAUCAUGGG | 1170 | asuscuugGfuGfUfCfgaaucaugggL96 | 2266 | sense | 21 |
| CCCAUGAUUCGACACCAAGAUCC | 1171 | csCfscauGfaUfUfcgacAfcCfaagauscsc | 2267 | antisense | 23 |
| UCUUGGUGUCGAAUCAUGGGG | 1172 | uscsuuggUfgUfCfGfaaucauggggL96 | 2268 | sense | 21 |
| CCCCAUGAUUCGACACCAAGAUC | 1173 | csCfsccaUfgAfUfucgaCfaCfcaagasusc | 2269 | antisense | 23 |
| UGGGAUCUUGGUGUCGAAUCA | 1174 | usgsggauCfuUfGfGfugucgaaucaL96 | 2270 | sense | 21 |
| UGAUUCGACACCAAGAUCCCAUU | 1175 | usGfsauuCfgAfCfaccaAfgAfucccasusu | 2271 | antisense | 23 |
| AUGGGAUCUUGGUGUCGAAUC | 1176 | asusgggaUfcUfUfGfgugucgaaucL96 | 2272 | sense | 21 |
| GAUUCGACACCAAGAUCCCAUUC | 1177 | gsAfsuucGfaCfAfccaaGfaUfcccaususc | 2273 | antisense | 23 |
| GCUACAAGGCCAUAUUUGUGA | 1178 | gscsuacaAfgGfCfCfauauuugugaL96 | 2274 | sense | 21 |
| UCACAAAUAUGGCCUUGUAGCCC | 1179 | usCfsacaAfaUfAfuggcCfuUfguagescsc | 2275 | antisense | 23 |
| CUACAAGGCCAUAUUUGUGAC | 1180 | csusacaaGfgCfCfAfuauuugugacL96 | 2276 | sense | 21 |
| GUCACAAAUAUGGCCUUGUAGCC | 1181 | gsUfscacAfaAfUfauggCfcUfuguagscsc | 2277 | antisense | 23 |
| AUGGGCUACAAGGCCAUAUUU | 1182 | asusgggcUfaCfAfAfggccauauuuL96 | 2278 | sense | 21 |
| AAAUAUGGCCUUGUAGCCCAUCU | 1183 | asAfsauaUfgGfCfcuugUfaGfcccauscsu | 2279 | antisense | 23 |
| GAUGGGCUACAAGGCCAUAUU | 1184 | gsasugggCfuAfCfAfaggccauauuL96 | 2280 | sense | 21 |
| AAUAUGGCCUUGUAGCCCAUCUU | 1185 | asAfsuauGfgCfCfuuguAfgCfccaucsusu | 2281 | antisense | 23 |
| ACUGGAGAGAAUUGGAAUGGG | 1186 | ascsuggaGfaGfAfAfuuggaaugggL96 | 2282 | sense | 21 |
| CCCAUUCCAAUUCUCUCCAGUGC | 1187 | csCfscauUfcCfAfauucUfcUfccagusgsc | 2283 | antisense | 23 |
| CUGGAGAGAAUUGGAAUGGGU | 1188 | csusggagAfgAfAfUfuggaauggguL96 | 2284 | sense | 21 |
| ACCCAUUCCAAUUCUCUCCAGUG | 1189 | asCfsccaUfuCfCfaauuCfuCfuccagsusg | 2285 | antisense | 23 |
| UAGCACUGGAGAGAAUUGGAA | 1190 | usasgcacUfgGfAfGfagaauuggaaL96 | 2286 | sense | 21 |
| UUCCAAUUCUCUCCAGUGCUACC | 1191 | usUfsccaAfuUfCfucucCfaGfugcuascsc | 2287 | antisense | 23 |
| GUAGCACUGGAGAGAAUUGGA | 1192 | gsusagcaCfuGfGfAfgagaauuggaL96 | 2288 | sense | 21 |
| UCCAAUUCUCUCCAGUGCUACCU | 1193 | usCfscaaUfuCfUfcuccAfgUfgcuacscsu | 2289 | antisense | 23 |
| ACAGUGGACACACCUUACCUG | 1194 | ascsagugGfaCfAfCfaccuuaccugL96 | 2290 | sense | 21 |
| CAGGUAAGGUGUGUCCACUGUCA | 1195 | csAfsgguAfaGfGfugugUfcCfacuguscsa | 2291 | antisense | 23 |
| CAGUGGACACACCUUACCUGG | 1196 | csasguggAfcAfCfAfccuuaccuggL96 | 2292 | sense | 21 |
| CCAGGUAAGGUGUGUCCACUGUC | 1197 | csCfsaggUfaAfGfguguGfuCfcacugsusc | 2293 | antisense | 23 |
| UGUGACAGUGGACACACCUUA | 1198 | usgsugacAfgUfGfGfacacaccuuaL96 | 2294 | sense | 21 |
| UAAGGUGUGUCCACUGUCACAAA | 1199 | usAfsaggUfgUfGfuccaCfuGfucacasasa | 2295 | antisense | 23 |
| UUGUGACAGUGGACACACCUU | 1200 | ususgugaCfaGfUfGfgacacaccuuL96 | 2296 | sense | 21 |
| AAGGUGUGUCCACUGUCACAAAU | 1201 | asAfsgguGfuGfUfccacUfgUfcacaasasu | 2297 | antisense | 23 |
| GAAGACUGACAUCAUUGCCAA | 1202 | gsasagacUfgAfCfAfucauugccaaL96 | 2298 | sense | 21 |
| UUGGCAAUGAUGUCAGUCUUCUC | 1203 | usUfsggcAfaUfGfauguCfaGfucuucsusc | 2299 | antisense | 23 |
| AAGACUGACAUCAUUGCCAAU | 1204 | asasgacuGfaCfAfUfcauugccaauL96 | 2300 | sense | 21 |
| AUUGGCAAUGAUGUCAGUCUUCU | 1205 | asUfsuggCfaAfUfgaugUfcAfgucuuscsu | 2301 | antisense | 23 |
| CUGAGAAGACUGACAUCAUUG | 1206 | csusgagaAfgAfCfUfgacaucauugL96 | 2302 | sense | 21 |
| CAAUGAUGUCAGUCUUCUCAGCC | 1207 | csAfsaugAfuGfUfcaguCfuUfcucagscsc | 2303 | antisense | 23 |
| GCUGAGAAGACUGACAUCAUU | 1208 | gscsugagAfaGfAfCfugacaucauuL96 | 2304 | sense | 21 |
| AAUGAUGUCAGUCUUCUCAGCCA | 1209 | asAfsugaUfgUfCfagucUfuCfucagcscsa | 2305 | antisense | 23 |
| GCUCAGGUUCAAAGUGUUGGU | 1210 | gscsucagGfuUfCfAfaaguguugguL96 | 2306 | sense | 21 |
| ACCAACACUUUGAACCUGAGCUU | 1211 | asCfscaaCfaCfUfuugaAfcCfugagcsusu | 2307 | antisense | 23 |
| CUCAGGUUCAAAGUGUUGGUA | 1212 | csuscaggUfuCfAfAfaguguugguaL96 | 2308 | sense | 21 |
| UACCAACACUUUGAACCUGAGCU | 1213 | usAfsccaAfcAfCfuuugAfaCfcugagscsu | 2309 | antisense | 23 |
| GUAAGCUCAGGUUCAAAGUGU | 1214 | gsusaagcUfcAfGfGfuucaaaguguL96 | 2310 | sense | 21 |
| ACACUUUGAACCUGAGCUUACAA | 1215 | asCfsacuUfuGfAfaccuGfaGfcuuacsasa | 2311 | antisense | 23 |
| UGUAAGCUCAGGUUCAAAGUG | 1216 | usgsuaagCfuCfAfGfguucaaagugL96 | 2312 | sense | 21 |
| CACUUUGAACCUGAGCUUACAAU | 1217 | csAfscuuUfgAfAfccugAfgCfuuacasasu | 2313 | antisense | 23 |
| AUGUAUUACUUGACAAAGAGA | 1218 | asusguauUfaCfUfUfgacaaagagaL96 | 2314 | sense | 21 |
| UCUCUUUGUCAAGUAAUACAUGC | 1219 | usCfsucuUfuGfUfcaagUfaAfuacausgsc | 2315 | antisense | 23 |
| UGUAUUACUUGACAAAGAGAC | 1220 | usgsuauuAfcUfUfGfacaaagagacL96 | 2316 | sense | 21 |
| GUCUCUUUGUCAAGUAAUACAUG | 1221 | gsUfscucUfuUfGfucaaGfuAfauacasusg | 2317 | antisense | 23 |
| CAGCAUGUAUUACUUGACAAA | 1222 | csasgcauGfuAfUfUfacuugacaaaL96 | 2318 | sense | 21 |
| UUUGUCAAGUAAUACAUGCUGAA | 1223 | usUfsuguCfaAfGfuaauAfcAfugcugsasa | 2319 | antisense | 23 |
| UCAGCAUGUAUUACUUGACAA | 1224 | uscsagcaUfgUfAfUfuacuugacaaL96 | 2320 | sense | 21 |
| UUGUCAAGUAAUACAUGCUGAAA | 1225 | usUfsgucAfaGfUfaauaCfaUfgcugasasa | 2321 | antisense | 23 |
| CUGCAACUGUAUAUCUACAAG | 1226 | csusgcaaCfuGfUfAfuaucuacaagL96 | 2322 | sense | 21 |
| CUUGUAGAUAUACAGUUGCAGCC | 1227 | csUfsuguAfgAfUfauacAfgUfugcagscsc | 2323 | antisense | 23 |
| UGCAACUGUAUAUCUACAAGG | 1228 | usgscaacUfgUfAfUfaucuacaaggL96 | 2324 | sense | 21 |
| CCUUGUAGAUAUACAGUUGCAGC | 1229 | csCfsuugUfaGfAfuauaCfaGfuugcasgsc | 2325 | antisense | 23 |
| UUGGCUGCAACUGUAUAUCUA | 1230 | ususggcuGfcAfAfCfuguauaucuaL96 | 2326 | sense | 21 |
| UAGAUAUACAGUUGCAGCCAACG | 1231 | usAfsgauAfuAfCfaguuGfcAfgccaascsg | 2327 | antisense | 23 |
| GUUGGCUGCAACUGUAUAUCU | 1232 | gsusuggcUfgCfAfAfcuguauaucuL96 | 2328 | sense | 21 |
| AGAUAUACAGUUGCAGCCAACGA | 1233 | asGfsauaUfaCfAfguugCfaGfccaacsgsa | 2329 | antisense | 23 |
| CAAAUGAUGAAGAAACUUUGG | 1234 | csasaaugAfuGfAfAfgaaacuuuggL96 | 2330 | sense | 21 |
| CCAAAGUUUCUUCAUCAUUUGCC | 1235 | csCfsaaaGfuUfUfcuucAfuCfauuugscsc | 2331 | antisense | 23 |
| AAAUGAUGAAGAAACUUUGGC | 1236 | asasaugaUfgAfAfGfaaacuuuggcL96 | 2332 | sense | 21 |
| GCCAAAGUUUCUUCAUCAUUUGC | 1237 | gsCfscaaAfgUfUfucuuCfaUfcauuusgsc | 2333 | antisense | 23 |
| GGGGCAAAUGAUGAAGAAACU | 1238 | gsgsggcaAfaUfGfAfugaagaaacuL96 | 2334 | sense | 21 |
| AGUUUCUUCAUCAUUUGCCCCAG | 1239 | asGfsuuuCfuUfCfaucaUfuUfgccccsasg | 2335 | antisense | 23 |
| UGGGGCAAAUGAUGAAGAAAC | 1240 | usgsgggcAfaAfUfGfaugaagaaacL96 | 2336 | sense | 21 |
| GUUUCUUCAUCAUUUGCCCCAGA | 1241 | gsUfsuucUfuCfAfucauUfuGfccccasgsa | 2337 | antisense | 23 |
| CAAAGGGUGUCGUUCUUUUCC | 1242 | csasaaggGfuGfUfCfguucuuuuccL96 | 2338 | sense | 21 |
| GGAAAAGAACGACACCCUUUGUA | 1243 | gsGfsaaaAfgAfAfcgacAfcCfcuuugsusa | 2339 | antisense | 23 |
| AAAGGGUGUCGUUCUUUUCCA | 1244 | asasagggUfgUfCfGfuucuuuuccaL96 | 2340 | sense | 21 |
| UGGAAAAGAACGACACCCUUUGU | 1245 | usGfsgaaAfaGfAfacgaCfaCfccuuusgsu | 2341 | antisense | 23 |
| AAUACAAAGGGUGUCGUUCUU | 1246 | asasuacaAfaGfGfGfugucguucuuL96 | 2342 | sense | 21 |
| AAGAACGACACCCUUUGUAUUGA | 1247 | asAfsgaaCfgAfCfacccUfuUfguauusgsa | 2343 | antisense | 23 |
| CAAUACAAAGGGUGUCGUUCU | 1248 | csasauacAfaAfGfGfgugucguucuL96 | 2344 | sense | 21 |
| AGAACGACACCCUUUGUAUUGAA | 1249 | asGfsaacGfaCfAfcccuUfuGfuauugsasa | 2345 | antisense | 23 |
| AAAGGCACUGAUGUUCUGAAA | 1250 | asasaggcAfcUfGfAfuguucugaaaL96 | 2346 | sense | 21 |
| UUUCAGAACAUCAGUGCCUUUCC | 1251 | usUfsucaGfaAfCfaucaGfuGfccuuuscsc | 2347 | antisense | 23 |
| AAGGCACUGAUGUUCUGAAAG | 1252 | asasggcaCfuGfAfUfguucugaaagL96 | 2348 | sense | 21 |
| CUUUCAGAACAUCAGUGCCUUUC | 1253 | csUfsuucAfgAfAfcaucAfgUfgccuususc | 2349 | antisense | 23 |
| GCGGAAAGGCACUGAUGUUCU | 1254 | gscsggaaAfgGfCfAfcugauguucuL96 | 2350 | sense | 21 |
| AGAACAUCAGUGCCUUUCCGCAC | 1255 | asGfsaacAfuCfAfgugcCfuUfuccgcsasc | 2351 | antisense | 23 |
| UGCGGAAAGGCACUGAUGUUC | 1256 | usgscggaAfaGfGfCfacugauguucL96 | 2352 | sense | 21 |
| GAACAUCAGUGCCUUUCCGCACA | 1257 | gsAfsacaUfcAfGfugccUfuUfccgcascsa | 2353 | antisense | 23 |
| AAGGAUGCUCCGGAAUGUUGC | 1258 | asasggauGfcUfCfCfggaauguugcL96 | 2354 | sense | 21 |
| GCAACAUUCCGGAGCAUCCUUGG | 1259 | gsCfsaacAfuUfCfcggaGfcAfuccuusgsg | 2355 | antisense | 23 |
| AGGAUGCUCCGGAAUGUUGCU | 1260 | asgsgaugCfuCfCfGfgaauguugcuL96 | 2356 | sense | 21 |
| AGCAACAUUCCGGAGCAUCCUUG | 1261 | asGfscaaCfaUfUfccggAfgCfauccususg | 2357 | antisense | 23 |
| AUCCAAGGAUGCUCCGGAAUG | 1262 | asusccaaGfgAfUfGfcuccggaaugL96 | 2358 | sense | 21 |
| CAUUCCGGAGCAUCCUUGGAUAC | 1263 | csAfsuucCfgGfAfgcauCfcUfuggausasc | 2359 | antisense | 23 |
| UAUCCAAGGAUGCUCCGGAAU | 1264 | usasuccaAfgGfAfUfgcuccggaauL96 | 2360 | sense | 21 |
| AUUCCGGAGCAUCCUUGGAUACA | 1265 | asUfsuccGfgAfGfcaucCfuUfggauascsa | 2361 | antisense | 23 |
| AAUGGGUGGCGGUAAUUGGUG | 1266 | asasugggUfgGfCfGfguaauuggugL96 | 2362 | sense | 21 |
| CACCAAUUACCGCCACCCAUUCC | 1267 | csAfsccaAfuUfAfccgcCfaCfccauuscsc | 2363 | antisense | 23 |
| AUGGGUGGCGGUAAUUGGUGA | 1268 | asusggguGfgCfGfGfuaauuggugaL96 | 2364 | sense | 21 |
| UCACCAAUUACCGCCACCCAUUC | 1269 | usCfsaccAfaUfUfaccgCfcAfcccaususc | 2365 | antisense | 23 |
| UUGGAAUGGGUGGCGGUAAUU | 1270 | ususggaaUfgGfGfUfggcgguaauuL96 | 2366 | sense | 21 |
| AAUUACCGCCACCCAUUCCAAUU | 1271 | asAfsuuaCfcGfCfcaccCfaUfuccaasusu | 2367 | antisense | 23 |
| AUUGGAAUGGGUGGCGGUAAU | 1272 | asusuggaAfuGfGfGfuggcgguaauL96 | 2368 | sense | 21 |
| AUUACCGCCACCCAUUCCAAUUC | 1273 | asUfsuacCfgCfCfacccAfuUfccaaususc | 2369 | antisense | 23 |
| GGAAAGGCACUGAUGUUCUGA | 1274 | gsgsaaagGfcAfCfUfgauguucugaL96 | 2370 | sense | 21 |
| UCAGAACAUCAGUGCCUUUCCGC | 1275 | usCfsagaAfcAfUfcaguGfcCfuuuccsgsc | 2371 | antisense | 23 |
| GAAAGGCACUGAUGUUCUGAA | 1276 | gsasaaggCfaCfUfGfauguucugaaL96 | 2372 | sense | 21 |
| UUCAGAACAUCAGUGCCUUUCCG | 1277 | usUfscagAfaCfAfucagUfgCfcuuucscsg | 2373 | antisense | 23 |
| GUGCGGAAAGGCACUGAUGUU | 1278 | gsusgcggAfaAfGfGfcacugauguuL96 | 2374 | sense | 21 |
| AACAUCAGUGCCUUUCCGCACAC | 1279 | asAfscauCfaGfUfgccuUfuCfcgcacsasc | 2375 | antisense | 23 |
| UGUGCGGAAAGGCACUGAUGU | 1280 | usgsugcgGfaAfAfGfgcacugauguL96 | 2376 | sense | 21 |
| ACAUCAGUGCCUUUCCGCACACC | 1281 | asCfsaucAfgUfGfccuuUfcCfgcacascsc | 2377 | antisense | 23 |
| AAUUGUAAGCUCAGGUUCAAA | 1282 | asasuuguAfaGfCfUfcagguucaaaL96 | 2378 | sense | 21 |
| UUUGAACCUGAGCUUACAAUUUA | 1283 | usUfsugaAfcCfUfgagcUfuAfcaauususa | 2379 | antisense | 23 |
| AUUGUAAGCUCAGGUUCAAAG | 1284 | asusuguaAfgCfUfCfagguucaaagL96 | 2380 | sense | 21 |
| CUUUGAACCUGAGCUUACAAUUU | 1285 | csUfsuugAfaCfCfugagCfuUfacaaususu | 2381 | antisense | 23 |
| CUUAAAUUGUAAGCUCAGGUU | 1286 | csusuaaaUfuGfUfAfagcucagguuL96 | 2382 | sense | 21 |
| AACCUGAGCUUACAAUUUAAGAA | 1287 | asAfsccuGfaGfCfuuacAfaUfuuaagsasa | 2383 | antisense | 23 |
| UCUUAAAUUGUAAGCUCAGGU | 1288 | uscsuuaaAfuUfGfUfaagcucagguL96 | 2384 | sense | 21 |
| ACCUGAGCUUACAAUUUAAGAAC | 1289 | asCfscugAfgCfUfuacaAfuUfuaagasasc | 2385 | antisense | 23 |
| GCAAACACUAAGGUGAAAAGA | 1290 | gscsaaacAfcUfAfAfggugaaaagaL96 | 2386 | sense | 21 |
| UCUUUUCACCUUAGUGUUUGCUA | 1291 | usCfsuuuUfcAfCfcuuaGfuGfuuugcsusa | 2387 | antisense | 23 |
| CAAACACUAAGGUGAAAAGAU | 1292 | csasaacaCfuAfAfGfgugaaaagauL96 | 2388 | sense | 21 |
| AUCUUUUCACCUUAGUGUUUGCU | 1293 | asUfscuuUfuCfAfccuuAfgUfguuugscsu | 2389 | antisense | 23 |
| GGUAGCAAACACUAAGGUGAA | 1294 | gsgsuagcAfaAfCfAfcuaaggugaaL96 | 2390 | sense | 21 |
| UUCACCUUAGUGUUUGCUACCUC | 1295 | usUfscacCfuUfAfguguUfuGfcuaccsusc | 2391 | antisense | 23 |
| AGGUAGCAAACACUAAGGUGA | 1296 | asgsguagCfaAfAfCfacuaaggugaL96 | 2392 | sense | 21 |
| UCACCUUAGUGUUUGCUACCUCC | 1297 | usCfsaccUfuAfGfuguuUfgCfuaccuscsc | 2393 | antisense | 23 |
| AGGUAGCAAACACUAAGGUGA | 1298 | asgsguagCfaAfAfCfacuaaggugaL96 | 2394 | sense | 21 |
| UCACCUUAGUGUUUGCUACCUCC | 1299 | usCfsaccUfuAfGfuguuUfgCfuaccuscsc | 2395 | antisense | 23 |
| GGUAGCAAACACUAAGGUGAA | 1300 | gsgsuagcAfaAfCfAfcuaaggugaaL96 | 2396 | sense | 21 |
| UUCACCUUAGUGUUUGCUACCUC | 1301 | usUfscacCfuUfAfguguUfuGfcuaccsusc | 2397 | antisense | 23 |
| UUGGAGGUAGCAAACACUAAG | 1302 | ususggagGfuAfGfCfaaacacuaagL96 | 2398 | sense | 21 |
| CUUAGUGUUUGCUACCUCCAAUU | 1303 | csUfsuagUfgUfUfugcuAfcCfuccaasusu | 2399 | antisense | 23 |
| AUUGGAGGUAGCAAACACUAA | 1304 | asusuggaGfgUfAfGfcaaacacuaaL96 | 2400 | sense | 21 |
| UUAGUGUUUGCUACCUCCAAUUU | 1305 | usUfsaguGfuUfUfgcuaCfcUfccaaususu | 2401 | antisense | 23 |
| UAAAGUGCUGUAUCCUUUAGU | 1306 | usasaaguGfcUfGfUfauccuuuaguL96 | 2402 | sense | 21 |
| ACUAAAGGAUACAGCACUUUAGC | 1307 | asCfsuaaAfgGfAfuacaGfcAfcuuuasgsc | 2403 | antisense | 23 |
| AAAGUGCUGUAUCCUUUAGUA | 1308 | asasagugCfuGfUfAfuccuuuaguaL96 | 2404 | sense | 21 |
| UACUAAAGGAUACAGCACUUUAG | 1309 | usAfscuaAfaGfGfauacAfgCfacuuusasg | 2405 | antisense | 23 |
| AGGCUAAAGUGCUGUAUCCUU | 1310 | asgsgcuaAfaGfUfGfcuguauccuuL96 | 2406 | sense | 21 |
| AAGGAUACAGCACUUUAGCCUGC | 1311 | asAfsggaUfaCfAfgcacUfuUfagccusgsc | 2407 | antisense | 23 |
| CAGGCUAAAGUGCUGUAUCCU | 1312 | csasggcuAfaAfGfUfgcuguauccuL96 | 2408 | sense | 21 |
| AGGAUACAGCACUUUAGCCUGCC | 1313 | asGfsgauAfcAfGfcacuUfuAfgccugscsc | 2409 | antisense | 23 |
| AAGACAUUGGUGAGGAAAAAU | 1314 | asasgacaUfuGfGfUfgaggaaaaauL96 | 2410 | sense | 21 |
| AUUUUUCCUCACCAAUGUCUUGU | 1315 | asUfsuuuUfcCfUfcaccAfaUfgucuusgsu | 2411 | antisense | 23 |
| AGACAUUGGUGAGGAAAAAUC | 1316 | asgsacauUfgGfUfGfaggaaaaaucL96 | 2412 | sense | 21 |
| GAUUUUUCCUCACCAAUGUCUUG | 1317 | gsAfsuuuUfuCfCfucacCfaAfugucususg | 2413 | antisense | 23 |
| CGACAAGACAUUGGUGAGGAA | 1318 | csgsacaaGfaCfAfUfuggugaggaaL96 | 2414 | sense | 21 |
| UUCCUCACCAAUGUCUUGUCGAU | 1319 | usUfsccuCfaCfCfaaugUfcUfugucgsasu | 2415 | antisense | 23 |
| UCGACAAGACAUUGGUGAGGA | 1320 | uscsgacaAfgAfCfAfuuggugaggaL96 | 2416 | sense | 21 |
| UCCUCACCAAUGUCUUGUCGAUG | 1321 | usCfscucAfcCfAfauguCfuUfgucgasusg | 2417 | antisense | 23 |
| AAGAUGUCCUCGAGAUACUAA | 1322 | asasgaugUfcCfUfCfgagauacuaaL96 | 2418 | sense | 21 |
| UUAGUAUCUCGAGGACAUCUUGA | 1323 | usUfsaguAfuCfUfcgagGfaCfaucuusgsa | 2419 | antisense | 23 |
| AGAUGUCCUCGAGAUACUAAA | 1324 | asgsauguCfcUfCfGfagauacuaaaL96 | 2420 | sense | 21 |
| UUUAGUAUCUCGAGGACAUCUUG | 1325 | usUfsuagUfaUfCfucgaGfgAfcaucususg | 2421 | antisense | 23 |
| GUUCAAGAUGUCCUCGAGAUA | 1326 | gsusucaaGfaUfGfUfccucgagauaL96 | 2422 | sense | 21 |
| UAUCUCGAGGACAUCUUGAACAC | 1327 | usAfsucuCfgAfGfgacaUfcUfugaacsasc | 2423 | antisense | 23 |
| UGUUCAAGAUGUCCUCGAGAU | 1328 | usgsuucaAfgAfUfGfuccucgagauL96 | 2424 | sense | 21 |
| AUCUCGAGGACAUCUUGAACACC | 1329 | asUfscucGfaGfGfacauCfuUfgaacascsc | 2425 | antisense | 23 |
| GAGAAAGGUGUUCAAGAUGUC | 1330 | gsasgaaaGfgUfGfUfucaagaugucL96 | 2426 | sense | 21 |
| GACAUCUUGAACACCUUUCUCCC | 1331 | gsAfscauCfuUfGfaacaCfcUfuucucscsc | 2427 | antisense | 23 |
| AGAAAGGUGUUCAAGAUGUCC | 1332 | asgsaaagGfuGfUfUfcaagauguccL96 | 2428 | sense | 21 |
| GGACAUCUUGAACACCUUUCUCC | 1333 | gsGfsacaUfcUfUfgaacAfcCfuuucuscsc | 2429 | antisense | 23 |
| GGGGGAGAAAGGUGUUCAAGA | 1334 | gsgsgggaGfaAfAfGfguguucaagaL96 | 2430 | sense | 21 |
| UCUUGAACACCUUUCUCCCCCUG | 1335 | usCfsuugAfaCfAfccuuUfcUfcccccsusg | 2431 | antisense | 23 |
| AGGGGGAGAAAGGUGUUCAAG | 1336 | asgsggggAfgAfAfAfgguguucaagL96 | 2432 | sense | 21 |
| CUUGAACACCUUUCUCCCCCUGG | 1337 | csUfsugaAfcAfCfcuuuCfuCfccccusgsg | 2433 | antisense | 23 |
| GCUGGGAAGAUAUCAAAUGGC | 1338 | gscsugggAfaGfAfUfaucaaauggcL96 | 2434 | sense | 21 |
| GCCAUUUGAUAUCUUCCCAGCUG | 1339 | gsCfscauUfuGfAfuaucUfuCfccagcsusg | 2435 | antisense | 23 |
| CUGGGAAGAUAUCAAAUGGCU | 1340 | csusgggaAfgAfUfAfucaaauggcuL96 | 2436 | sense | 21 |
| AGCCAUUUGAUAUCUUCCCAGCU | 1341 | asGfsccaUfuUfGfauauCfuUfcccagscsu | 2437 | antisense | 23 |
| AUCAGCUGGGAAGAUAUCAAA | 1342 | asuscagcUfgGfGfAfagauaucaaaL96 | 2438 | sense | 21 |
| UUUGAUAUCUUCCCAGCUGAUAG | 1343 | usUfsugaUfaUfCfuuccCfaGfcugausasg | 2439 | antisense | 23 |
| UAUCAGCUGGGAAGAUAUCAA | 1344 | usasucagCfuGfGfGfaagauaucaaL96 | 2440 | sense | 21 |
| UUGAUAUCUUCCCAGCUGAUAGA | 1345 | usUfsgauAfuCfUfucccAfgCfugauasgsa | 2441 | antisense | 23 |
| UCUGUCGACUUCUGUUUUAGG | 1346 | uscsugucGfaCfUfUfcuguuuuaggL96 | 2442 | sense | 21 |
| CCUAAAACAGAAGUCGACAGAUC | 1347 | csCfsuaaAfaCfAfgaagUfcGfacagasusc | 2443 | antisense | 23 |
| CUGUCGACUUCUGUUUUAGGA | 1348 | csusgucgAfcUfUfCfuguuuuaggaL96 | 2444 | sense | 21 |
| UCCUAAAACAGAAGUCGACAGAU | 1349 | usCfscuaAfaAfCfagaaGfuCfgacagsasu | 2445 | antisense | 23 |
| CAGAUCUGUCGACUUCUGUUU | 1350 | csasgaucUfgUfCfGfacuucuguuuL96 | 2446 | sense | 21 |
| AAACAGAAGUCGACAGAUCUGUU | 1351 | asAfsacaGfaAfGfucgaCfaGfaucugsusu | 2447 | antisense | 23 |
| ACAGAUCUGUCGACUUCUGUU | 1352 | ascsagauCfuGfUfCfgacuucuguuL96 | 2448 | sense | 21 |
| AACAGAAGUCGACAGAUCUGUUU | 1353 | asAfscagAfaGfUfcgacAfgAfucugususu | 2449 | antisense | 23 |
| UACUUCUUUGAAUGUAGAUUU | 1354 | usascuucUfuUfGfAfauguagauuuL96 | 2450 | sense | 21 |
| AAAUCUACAUUCAAAGAAGUAUC | 1355 | asAfsaucUfaCfAfuucaAfaGfaaguasusc | 2451 | antisense | 23 |
| ACUUCUUUGAAUGUAGAUUUC | 1356 | ascsuucuUfuGfAfAfuguagauuucL96 | 2452 | sense | 21 |
| GAAAUCUACAUUCAAAGAAGUAU | 1357 | gsAfsaauCfuAfCfauucAfaAfgaagusasu | 2453 | antisense | 23 |
| GUGAUACUUCUUUGAAUGUAG | 1358 | gsusgauaCfuUfCfUfuugaauguagL96 | 2454 | sense | 21 |
| CUACAUUCAAAGAAGUAUCACCA | 1359 | csUfsacaUfuCfAfaagaAfgUfaucacscsa | 2455 | antisense | 23 |
| GGUGAUACUUCUUUGAAUGUA | 1360 | gsgsugauAfcUfUfCfuuugaauguaL96 | 2456 | sense | 21 |
| UACAUUCAAAGAAGUAUCACCAA | 1361 | usAfscauUfcAfAfagaaGfuAfucaccsasa | 2457 | antisense | 23 |
| UGGGAAGAUAUCAAAUGGCUG | 1362 | usgsggaaGfaUfAfUfcaaauggcugL96 | 2458 | sense | 21 |
| CAGCCAUUUGAUAUCUUCCCAGC | 1363 | csAfsgccAfuUfUfgauaUfcUfucccasgsc | 2459 | antisense | 23 |
| GGGAAGAUAUCAAAUGGCUGA | 1364 | gsgsgaagAfuAfUfCfaaauggcugaL96 | 2460 | sense | 21 |
| UCAGCCAUUUGAUAUCUUCCCAG | 1365 | usCfsagcCfaUfUfugauAfuCfuucccsasg | 2461 | antisense | 23 |
| CAGCUGGGAAGAUAUCAAAUG | 1366 | csasgcugGfgAfAfGfauaucaaaugL96 | 2462 | sense | 21 |
| CAUUUGAUAUCUUCCCAGCUGAU | 1367 | csAfsuuuGfaUfAfucuuCfcCfagcugsasu | 2463 | antisense | 23 |
| UCAGCUGGGAAGAUAUCAAAU | 1368 | uscsagcuGfgGfAfAfgauaucaaauL96 | 2464 | sense | 21 |
| AUUUGAUAUCUUCCCAGCUGAUA | 1369 | asUfsuugAfuAfUfcuucCfcAfgcugasusa | 2465 | antisense | 23 |
| UCCAAAGUCUAUAUAUGACUA | 1370 | uscscaaaGfuCfUfAfuauaugacuaL96 | 2466 | sense | 21 |
| UAGUCAUAUAUAGACUUUGGAAG | 1371 | usAfsgucAfuAfUfauagAfcUfuuggasasg | 2467 | antisense | 23 |
| CCAAAGUCUAUAUAUGACUAU | 1372 | cscsaaagUfcUfAfUfauaugacuauL96 | 2468 | sense | 21 |
| AUAGUCAUAUAUAGACUUUGGAA | 1373 | asUfsaguCfaUfAfuauaGfaCfuuuggsasa | 2469 | antisense | 23 |
| UACUUCCAAAGUCUAUAUAUG | 1374 | usascuucCfaAfAfGfucuauauaugL96 | 2470 | sense | 21 |
| CAUAUAUAGACUUUGGAAGUACU | 1375 | csAfsuauAfuAfGfacuuUfgGfaaguascsu | 2471 | antisense | 23 |
| GUACUUCCAAAGUCUAUAUAU | 1376 | gsusacuuCfcAfAfAfgucuauauauL96 | 2472 | sense | 21 |
| AUAUAUAGACUUUGGAAGUACUG | 1377 | asUfsauaUfaGfAfcuuuGfgAfaguacsusg | 2473 | antisense | 23 |
| UUAUGAACAACAUGCUAAAUC | 1378 | ususaugaAfcAfAfCfaugcuaaaucL96 | 2474 | sense | 21 |
| GAUUUAGCAUGUUGUUCAUAAUC | 1379 | gsAfsuuuAfgCfAfuguuGfuUfcauaasusc | 2475 | antisense | 23 |
| UAUGAACAACAUGCUAAAUCA | 1380 | usasugaaCfaAfCfAfugcuaaaucaL96 | 2476 | sense | 21 |
| UGAUUUAGCAUGUUGUUCAUAAU | 1381 | usGfsauuUfaGfCfauguUfgUfucauasasu | 2477 | antisense | 23 |
| AUGAUUAUGAACAACAUGCUA | 1382 | asusgauuAfuGfAfAfcaacaugcuaL96 | 2478 | sense | 21 |
| UAGCAUGUUGUUCAUAAUCAUUG | 1383 | usAfsgcaUfgUfUfguucAfuAfaucaususg | 2479 | antisense | 23 |
| AAUGAUUAUGAACAACAUGCU | 1384 | asasugauUfaUfGfAfacaacaugcuL96 | 2480 | sense | 21 |
| AGCAUGUUGUUCAUAAUCAUUGA | 1385 | asGfscauGfuUfGfuucaUfaAfucauusgsa | 2481 | antisense | 23 |
| AAUUCCCCACUUCAAUACAAA | 1386 | asasuuccCfcAfCfUfucaauacaaaL96 | 2482 | sense | 21 |
| UUUGUAUUGAAGUGGGGAAUUAC | 1387 | usUfsuguAfuUfGfaaguGfgGfgaauusasc | 2483 | antisense | 23 |
| AUUCCCCACUUCAAUACAAAG | 1388 | asusucccCfaCfUfUfcaauacaaagL96 | 2484 | sense | 21 |
| CUUUGUAUUGAAGUGGGGAAUUA | 1389 | csUfsuugUfaUfUfgaagUfgGfggaaususa | 2485 | antisense | 23 |
| CUGUAAUUCCCCACUUCAAUA | 1390 | csusguaaUfuCfCfCfcacuucaauaL96 | 2486 | sense | 21 |
| UAUUGAAGUGGGGAAUUACAGAC | 1391 | usAfsuugAfaGfUfggggAfaUfuacagsasc | 2487 | antisense | 23 |
| UCUGUAAUUCCCCACUUCAAU | 1392 | uscsuguaAfuUfCfCfccacuucaauL96 | 2488 | sense | 21 |
| AUUGAAGUGGGGAAUUACAGACU | 1393 | asUfsugaAfgUfGfgggaAfuUfacagascsu | 2489 | antisense | 23 |
| UGAUGUGCGUAACAGAUUCAA | 1394 | usgsauguGfcGfUfAfacagauucaaL96 | 2490 | sense | 21 |
| UUGAAUCUGUUACGCACAUCAUC | 1395 | usUfsgaaUfcUfGfuuacGfcAfcaucasusc | 2491 | antisense | 23 |
| GAUGUGCGUAACAGAUUCAAA | 1396 | gsasugugCfgUfAfAfcagauucaaaL96 | 2492 | sense | 21 |
| UUUGAAUCUGUUACGCACAUCAU | 1397 | usUfsugaAfuCfUfguuaCfgCfacaucsasu | 2493 | antisense | 23 |
| UGGAUGAUGUGCGUAACAGAU | 1398 | usgsgaugAfuGfUfGfcguaacagauL96 | 2494 | sense | 21 |
| AUCUGUUACGCACAUCAUCCAGA | 1399 | asUfscugUfuAfCfgcacAfuCfauccasgsa | 2495 | antisense | 23 |
| CUGGAUGAUGUGCGUAACAGA | 1400 | csusggauGfaUfGfUfgcguaacagaL96 | 2496 | sense | 21 |
| UCUGUUACGCACAUCAUCCAGAC | 1401 | usCfsuguUfaCfGfcacaUfcAfuccagsasc | 2497 | antisense | 23 |
| GAAUGGGUGGCGGUAAUUGGU | 1402 | gsasauggGfuGfGfCfgguaauugguL96 | 2498 | sense | 21 |
| ACCAAUUACCGCCACCCAUUCCA | 1403 | asCfscaaUfuAfCfcgccAfcCfcauucscsa | 2499 | antisense | 23 |
| AAUGGGUGGCGGUAAUUGGUG | 1404 | asasugggUfgGfCfGfguaauuggugL96 | 2500 | sense | 21 |
| CACCAAUUACCGCCACCCAUUCC | 1405 | csAfsccaAfuUfAfccgcCfaCfccauuscsc | 2501 | antisense | 23 |
| AUUGGAAUGGGUGGCGGUAAU | 1406 | asusuggaAfuGfGfGfuggcgguaauL96 | 2502 | sense | 21 |
| AUUACCGCCACCCAUUCCAAUUC | 1407 | asUfsuacCfgCfCfacccAfuUfccaaususc | 2503 | antisense | 23 |
| AAUUGGAAUGGGUGGCGGUAA | 1408 | asasuuggAfaUfGfGfguggcgguaaL96 | 2504 | sense | 21 |
| UUACCGCCACCCAUUCCAAUUCU | 1409 | usUfsaccGfcCfAfcccaUfuCfcaauuscsu | 2505 | antisense | 23 |
| UCCGGAAUGUUGCUGAAACAG | 1410 | uscscggaAfuGfUfUfgcugaaacagL96 | 2506 | sense | 21 |
| CUGUUUCAGCAACAUUCCGGAGC | 1411 | csUfsguuUfcAfGfcaacAfuUfccggasgsc | 2507 | antisense | 23 |
| CCGGAAUGUUGCUGAAACAGA | 1412 | cscsggaaUfgUfUfGfcugaaacagaL96 | 2508 | sense | 21 |
| UCUGUUUCAGCAACAUUCCGGAG | 1413 | usCfsuguUfuCfAfgcaaCfaUfuccggsasg | 2509 | antisense | 23 |
| AUGCUCCGGAAUGUUGCUGAA | 1414 | asusgcucCfgGfAfAfuguugcugaaL96 | 2510 | sense | 21 |
| UUCAGCAACAUUCCGGAGCAUCC | 1415 | usUfscagCfaAfCfauucCfgGfagcauscsc | 2511 | antisense | 23 |
| GAUGCUCCGGAAUGUUGCUGA | 1416 | gsasugcuCfcGfGfAfauguugcugaL96 | 2512 | sense | 21 |
| UCAGCAACAUUCCGGAGCAUCCU | 1417 | usCfsagcAfaCfAfuuccGfgAfgcaucscsu | 2513 | antisense | 23 |
| UGUCCUCGAGAUACUAAAGGA | 1418 | usgsuccuCfgAfGfAfuacuaaaggaL96 | 2514 | sense | 21 |
| UCCUUUAGUAUCUCGAGGACAUC | 1419 | usCfscuuUfaGfUfaucuCfgAfggacasusc | 2515 | antisense | 23 |
| GUCCUCGAGAUACUAAAGGAA | 1420 | gsusccucGfaGfAfUfacuaaaggaaL96 | 2516 | sense | 21 |
| UUCCUUUAGUAUCUCGAGGACAU | 1421 | usUfsccuUfuAfGfuaucUfcGfaggacsasu | 2517 | antisense | 23 |
| AAGAUGUCCUCGAGAUACUAA | 1422 | asasgaugUfcCfUfCfgagauacuaaL96 | 2518 | sense | 21 |
| UUAGUAUCUCGAGGACAUCUUGA | 1423 | usUfsaguAfuCfUfcgagGfaCfaucuusgsa | 2519 | antisense | 23 |
| CAAGAUGUCCUCGAGAUACUA | 1424 | csasagauGfuCfCfUfcgagauacuaL96 | 2520 | sense | 21 |
| UAGUAUCUCGAGGACAUCUUGAA | 1425 | usAfsguaUfcUfCfgaggAfcAfucuugsasa | 2521 | antisense | 23 |
| ACAACAUGCUAAAUCAGUACU | 1426 | ascsaacaUfgCfUfAfaaucaguacuL96 | 2522 | sense | 21 |
| AGUACUGAUUUAGCAUGUUGUUC | 1427 | asGfsuacUfgAfUfuuagCfaUfguugususc | 2523 | antisense | 23 |
| CAACAUGCUAAAUCAGUACUU | 1428 | csasacauGfcUfAfAfaucaguacuuL96 | 2524 | sense | 21 |
| AAGUACUGAUUUAGCAUGUUGUU | 1429 | asAfsguaCfuGfAfuuuaGfcAfuguugsusu | 2525 | antisense | 23 |
| AUGAACAACAUGCUAAAUCAG | 1430 | asusgaacAfaCfAfUfgcuaaaucagL96 | 2526 | sense | 21 |
| CUGAUUUAGCAUGUUGUUCAUAA | 1431 | csUfsgauUfuAfGfcaugUfuGfuucausasa | 2527 | antisense | 23 |
| UAUGAACAACAUGCUAAAUCA | 1432 | usasugaaCfaAfCfAfugcuaaaucaL96 | 2528 | sense | 21 |
| UGAUUUAGCAUGUUGUUCAUAAU | 1433 | usGfsauuUfaGfCfauguUfgUfucauasasu | 2529 | antisense | 23 |
| GCCAAGGCUGUGUUUGUGGGG | 1434 | gscscaagGfcUfGfUfguuuguggggL96 | 2530 | sense | 21 |
| CCCCACAAACACAGCCUUGGCGC | 1435 | csCfsccaCfaAfAfcacaGfcCfuuggcsgsc | 2531 | antisense | 23 |
| CCAAGGCUGUGUUUGUGGGGA | 1436 | cscsaaggCfuGfUfGfuuuguggggaL96 | 2532 | sense | 21 |
| UCCCCACAAACACAGCCUUGGCG | 1437 | usCfscccAfcAfAfacacAfgCfcuuggscsg | 2533 | antisense | 23 |
| UGGCGCCAAGGCUGUGUUUGU | 1438 | usgsgcgcCfaAfGfGfcuguguuuguL96 | 2534 | sense | 21 |
| ACAAACACAGCCUUGGCGCCAAG | 1439 | asCfsaaaCfaCfAfgccuUfgGfcgccasasg | 2535 | antisense | 23 |
| UUGGCGCCAAGGCUGUGUUUG | 1440 | ususggcgCfcAfAfGfgcuguguuugL96 | 2536 | sense | 21 |
| CAAACACAGCCUUGGCGCCAAGA | 1441 | csAfsaacAfcAfGfccuuGfgCfgccaasgsa | 2537 | antisense | 23 |
| UGAAAGCUCUGGCUCUUGGCG | 1442 | usgsaaagCfuCfUfGfgcucuuggcgL96 | 2538 | sense | 21 |
| CGCCAAGAGCCAGAGCUUUCAGA | 1443 | csGfsccaAfgAfGfccagAfgCfuuucasgsa | 2539 | antisense | 23 |
| GAAAGCUCUGGCUCUUGGCGC | 1444 | gsasaagcUfcUfGfGfcucuuggcgcL96 | 2540 | sense | 21 |
| GCGCCAAGAGCCAGAGCUUUCAG | 1445 | gsCfsgccAfaGfAfgccaGfaGfcuuucsasg | 2541 | antisense | 23 |
| GUUCUGAAAGCUCUGGCUCUU | 1446 | gsusucugAfaAfGfCfucuggcucuuL96 | 2542 | sense | 21 |
| AAGAGCCAGAGCUUUCAGAACAU | 1447 | asAfsgagCfcAfGfagcuUfuCfagaacsasu | 2543 | antisense | 23 |
| UGUUCUGAAAGCUCUGGCUCU | 1448 | usgsuucuGfaAfAfGfcucuggcucuL96 | 2544 | sense | 21 |
| AGAGCCAGAGCUUUCAGAACAUC | 1449 | asGfsagcCfaGfAfgcuuUfcAfgaacasusc | 2545 | antisense | 23 |
| CAGCCACUAUUGAUGUUCUGC | 1450 | csasgccaCfuAfUfUfgauguucugcL96 | 2546 | sense | 21 |
| GCAGAACAUCAAUAGUGGCUGGC | 1451 | gsCfsagaAfcAfUfcaauAfgUfggcugsgsc | 2547 | antisense | 23 |
| AGCCACUAUUGAUGUUCUGCC | 1452 | asgsccacUfaUfUfGfauguucugccL96 | 2548 | sense | 21 |
| GGCAGAACAUCAAUAGUGGCUGG | 1453 | gsGfscagAfaCfAfucaaUfaGfuggcusgsg | 2549 | antisense | 23 |
| GUGCCAGCCACUAUUGAUGUU | 1454 | gsusgccaGfcCfAfCfuauugauguuL96 | 2550 | sense | 21 |
| AACAUCAAUAGUGGCUGGCACCC | 1455 | asAfscauCfaAfUfagugGfcUfggcacscsc | 2551 | antisense | 23 |
| GGUGCCAGCCACUAUUGAUGU | 1456 | gsgsugccAfgCfCfAfcuauugauguL96 | 2552 | sense | 21 |
| ACAUCAAUAGUGGCUGGCACCCC | 1457 | asCfsaucAfaUfAfguggCfuGfgcaccscsc | 2553 | antisense | 23 |
| ACAAGGACCGAGAAGUCACCA | 1458 | ascsaaggAfcCfGfAfgaagucaccaL96 | 2554 | sense | 21 |
| UGGUGACUUCUCGGUCCUUGUAG | 1459 | usGfsgugAfcUfUfcucgGfuCfcuugusasg | 2555 | antisense | 23 |
| CAAGGACCGAGAAGUCACCAA | 1460 | csasaggaCfcGfAfGfaagucaccaaL96 | 2556 | sense | 21 |
| UUGGUGACUUCUCGGUCCUUGUA | 1461 | usUfsgguGfaCfUfucucGfgUfccuugsusa | 2557 | antisense | 23 |
| AUCUACAAGGACCGAGAAGUC | 1462 | asuscuacAfaGfGfAfccgagaagucL96 | 2558 | sense | 21 |
| GACUUCUCGGUCCUUGUAGAUAU | 1463 | gsAfscuuCfuCfGfguccUfuGfuagausasu | 2559 | antisense | 23 |
| UAUCUACAAGGACCGAGAAGU | 1464 | usasucuaCfaAfGfGfaccgagaaguL96 | 2560 | sense | 21 |
| ACUUCUCGGUCCUUGUAGAUAUA | 1465 | asCfsuucUfcGfGfuccuUfgUfagauasusa | 2561 | antisense | 23 |
| CAGAAUGUGAAAGUCAUCGAC | 1466 | csasgaauGfuGfAfAfagucaucgacL96 | 2562 | sense | 21 |
| GUCGAUGACUUUCACAUUCUGGC | 1467 | gsUfscgaUfgAfCfuuucAfcAfuucugsgsc | 2563 | antisense | 23 |
| AGAAUGUGAAAGUCAUCGACA | 1468 | asgsaaugUfgAfAfAfgucaucgacaL96 | 2564 | sense | 21 |
| UGUCGAUGACUUUCACAUUCUGG | 1469 | usGfsucgAfuGfAfcuuuCfaCfauucusgsg | 2565 | antisense | 23 |
| GUGCCAGAAUGUGAAAGUCAU | 1470 | gsusgccaGfaAfUfGfugaaagucauL96 | 2566 | sense | 21 |
| AUGACUUUCACAUUCUGGCACCC | 1471 | asUfsgacUfuUfCfacauUfcUfggcacscsc | 2567 | antisense | 23 |
| GGUGCCAGAAUGUGAAAGUCA | 1472 | gsgsugccAfgAfAfUfgugaaagucaL96 | 2568 | sense | 21 |
| UGACUUUCACAUUCUGGCACCCA | 1473 | usGfsacuUfuCfAfcauuCfuGfgcaccscsa | 2569 | antisense | 23 |
| AGAUGUCCUCGAGAUACUAAA | 1474 | asgsauguCfcUfCfGfagauacuaaaL96 | 2570 | sense | 21 |
| UUUAGUAUCUCGAGGACAUCUUG | 1475 | usUfsuagUfaUfCfucgaGfgAfcaucususg | 2571 | antisense | 23 |
| GAUGUCCUCGAGAUACUAAAG | 1476 | gsasugucCfuCfGfAfgauacuaaagL96 | 2572 | sense | 21 |
| CUUUAGUAUCUCGAGGACAUCUU | 1477 | csUfsuuaGfuAfUfcucgAfgGfacaucsusu | 2573 | antisense | 23 |
| UUCAAGAUGUCCUCGAGAUAC | 1478 | ususcaagAfuGfUfCfcucgagauacL96 | 2574 | sense | 21 |
| GUAUCUCGAGGACAUCUUGAACA | 1479 | gsUfsaucUfcGfAfggacAfuCfuugaascsa | 2575 | antisense | 23 |
| GUUCAAGAUGUCCUCGAGAUA | 1480 | gsusucaaGfaUfGfUfccucgagauaL96 | 2576 | sense | 21 |
| UAUCUCGAGGACAUCUUGAACAC | 1481 | usAfsucuCfgAfGfgacaUfcUfugaacsasc | 2577 | antisense | 23 |
| GUGGACUUGCUGCAUAUGUGG | 1482 | gsusggacUfuGfCfUfgcauauguggL96 | 2578 | sense | 21 |
| CCACAUAUGCAGCAAGUCCACUG | 1483 | csCfsacaUfaUfGfcagcAfaGfuccacsusg | 2579 | antisense | 23 |
| UGGACUUGCUGCAUAUGUGGC | 1484 | usgsgacuUfgCfUfGfcauauguggcL96 | 2580 | sense | 21 |
| GCCACAUAUGCAGCAAGUCCACU | 1485 | gsCfscacAfuAfUfgcagCfaAfguccascsu | 2581 | antisense | 23 |
| GACAGUGGACUUGCUGCAUAU | 1486 | gsascaguGfgAfCfUfugcugcauauL96 | 2582 | sense | 21 |
| AUAUGCAGCAAGUCCACUGUCGU | 1487 | asUfsaugCfaGfCfaaguCfcAfcugucsgsu | 2583 | antisense | 23 |
| CGACAGUGGACUUGCUGCAUA | 1488 | csgsacagUfgGfAfCfuugcugcauaL96 | 2584 | sense | 21 |
| UAUGCAGCAAGUCCACUGUCGUC | 1489 | usAfsugcAfgCfAfagucCfaCfugucgsusc | 2585 | antisense | 23 |
| AACCAGUACUUUAUCAUUUUC | 1490 | asasccagUfaCfUfUfuaucauuuucL96 | 2586 | sense | 21 |
| GAAAAUGAUAAAGUACUGGUUUC | 1491 | gsAfsaaaUfgAfUfaaagUfaCfugguususc | 2587 | antisense | 23 |
| ACCAGUACUUUAUCAUUUUCU | 1492 | ascscaguAfcUfUfUfaucauuuucuL96 | 2588 | sense | 21 |
| AGAAAAUGAUAAAGUACUGGUUU | 1493 | asGfsaaaAfuGfAfuaaaGfuAfcuggususu | 2589 | antisense | 23 |
| UUGAAACCAGUACUUUAUCAU | 1494 | ususgaaaCfcAfGfUfacuuuaucauL96 | 2590 | sense | 21 |
| AUGAUAAAGUACUGGUUUCAAAA | 1495 | asUfsgauAfaAfGfuacuGfgUfuucaasasa | 2591 | antisense | 23 |
| UUUGAAACCAGUACUUUAUCA | 1496 | ususugaaAfcCfAfGfuacuuuaucaL96 | 2592 | sense | 21 |
| UGAUAAAGUACUGGUUUCAAAAU | 1497 | usGfsauaAfaGfUfacugGfuUfucaaasasu | 2593 | antisense | 23 |
| CGAGAAGUCACCAAGAAGCUA | 1498 | csgsagaaGfuCfAfCfcaagaagcuaL96 | 2594 | sense | 21 |
| UAGCUUCUUGGUGACUUCUCGGU | 1499 | usAfsgcuUfcUfUfggugAfcUfucucgsgsu | 2595 | antisense | 23 |
| GAGAAGUCACCAAGAAGCUAG | 1500 | gsasgaagUfcAfCfCfaagaagcuagL96 | 2596 | sense | 21 |
| CUAGCUUCUUGGUGACUUCUCGG | 1501 | csUfsagcUfuCfUfugguGfaCfuucucsgsg | 2597 | antisense | 23 |
| GGACCGAGAAGUCACCAAGAA | 1502 | gsgsaccgAfgAfAfGfucaccaagaaL96 | 2598 | sense | 21 |
| UUCUUGGUGACUUCUCGGUCCUU | 1503 | usUfscuuGfgUfGfacuuCfuCfgguccsusu | 2599 | antisense | 23 |
| AGGACCGAGAAGUCACCAAGA | 1504 | asgsgaccGfaGfAfAfgucaccaagaL96 | 2600 | sense | 21 |
| UCUUGGUGACUUCUCGGUCCUUG | 1505 | usCfsuugGfuGfAfcuucUfcGfguccususg | 2601 | antisense | 23 |
| UCAAAGUGUUGGUAAUGCCUG | 1506 | uscsaaagUfgUfUfGfguaaugccugL96 | 2602 | sense | 21 |
| CAGGCAUUACCAACACUUUGAAC | 1507 | csAfsggcAfuUfAfccaaCfaCfuuugasasc | 2603 | antisense | 23 |
| CAAAGUGUUGGUAAUGCCUGA | 1508 | csasaaguGfuUfGfGfuaaugccugaL96 | 2604 | sense | 21 |
| UCAGGCAUUACCAACACUUUGAA | 1509 | usCfsaggCfaUfUfaccaAfcAfcuuugsasa | 2605 | antisense | 23 |
| AGGUUCAAAGUGUUGGUAAUG | 1510 | asgsguucAfaAfGfUfguugguaaugL96 | 2606 | sense | 21 |
| CAUUACCAACACUUUGAACCUGA | 1511 | csAfsuuaCfcAfAfcacuUfuGfaaccusgsa | 2607 | antisense | 23 |
| CAGGUUCAAAGUGUUGGUAAU | 1512 | csasgguuCfaAfAfGfuguugguaauL96 | 2608 | sense | 21 |
| AUUACCAACACUUUGAACCUGAG | 1513 | asUfsuacCfaAfCfacuuUfgAfaccugsasg | 2609 | antisense | 23 |
| UAUUACUUGACAAAGAGACAC | 1514 | usasuuacUfuGfAfCfaaagagacacL96 | 2610 | sense | 21 |
| GUGUCUCUUUGUCAAGUAAUACA | 1515 | gsUfsgucUfcUfUfugucAfaGfuaauascsa | 2611 | antisense | 23 |
| AUUACUUGACAAAGAGACACU | 1516 | asusuacuUfgAfCfAfaagagacacuL96 | 2612 | sense | 21 |
| AGUGUCUCUUUGUCAAGUAAUAC | 1517 | asGfsuguCfuCfUfuuguCfaAfguaausasc | 2613 | antisense | 23 |
| CAUGUAUUACUUGACAAAGAG | 1518 | csasuguaUfuAfCfUfugacaaagagL96 | 2614 | sense | 21 |
| CUCUUUGUCAAGUAAUACAUGCU | 1519 | csUfscuuUfgUfCfaaguAfaUfacaugscsu | 2615 | antisense | 23 |
| GCAUGUAUUACUUGACAAAGA | 1520 | gscsauguAfuUfAfCfuugacaaagaL96 | 2616 | sense | 21 |
| UCUUUGUCAAGUAAUACAUGCUG | 1521 | usCfsuuuGfuCfAfaguaAfuAfcaugcsusg | 2617 | antisense | 23 |
| AAAGUCAUCGACAAGACAUUG | 1522 | asasagucAfuCfGfAfcaagacauugL96 | 2618 | sense | 21 |
| CAAUGUCUUGUCGAUGACUUUCA | 1523 | csAfsaugUfcUfUfgucgAfuGfacuuuscsa | 2619 | antisense | 23 |
| AAGUCAUCGACAAGACAUUGG | 1524 | asasgucaUfcGfAfCfaagacauuggL96 | 2620 | sense | 21 |
| CCAAUGUCUUGUCGAUGACUUUC | 1525 | csCfsaauGfuCfUfugucGfaUfgacuususc | 2621 | antisense | 23 |
| UGUGAAAGUCAUCGACAAGAC | 1526 | usgsugaaAfgUfCfAfucgacaagacL96 | 2622 | sense | 21 |
| GUCUUGUCGAUGACUUUCACAUU | 1527 | gsUfscuuGfuCfGfaugaCfuUfucacasusu | 2623 | antisense | 23 |
| AUGUGAAAGUCAUCGACAAGA | 1528 | asusgugaAfaGfUfCfaucgacaagaL96 | 2624 | sense | 21 |
| UCUUGUCGAUGACUUUCACAUUC | 1529 | usCfsuugUfcGfAfugacUfuUfcacaususc | 2625 | antisense | 23 |
| AUAUGUGGCUAAAGCAAUAGA | 1530 | asusauguGfgCfUfAfaagcaauagaL96 | 2626 | sense | 21 |
| UCUAUUGCUUUAGCCACAUAUGC | 1531 | usCfsuauUfgCfUfuuagCfcAfcauausgsc | 2627 | antisense | 23 |
| UAUGUGGCUAAAGCAAUAGAC | 1532 | usasugugGfcUfAfAfagcaauagacL96 | 2628 | sense | 21 |
| GUCUAUUGCUUUAGCCACAUAUG | 1533 | gsUfscuaUfuGfCfuuuaGfcCfacauasusg | 2629 | antisense | 23 |
| CUGCAUAUGUGGCUAAAGCAA | 1534 | csusgcauAfuGfUfGfgcuaaagcaaL96 | 2630 | sense | 21 |
| UUGCUUUAGCCACAUAUGCAGCA | 1535 | usUfsgcuUfuAfGfccacAfuAfugcagscsa | 2631 | antisense | 23 |
| GCUGCAUAUGUGGCUAAAGCA | 1536 | gscsugcaUfaUfGfUfggcuaaagcaL96 | 2632 | sense | 21 |
| UGCUUUAGCCACAUAUGCAGCAA | 1537 | usGfscuuUfaGfCfcacaUfaUfgcagcsasa | 2633 | antisense | 23 |
| AGACGACAGUGGACUUGCUGC | 1538 | asgsacgaCfaGfUfGfgacuugcugcL96 | 2634 | sense | 21 |
| GCAGCAAGUCCACUGUCGUCUCC | 1539 | gsCfsagcAfaGfUfccacUfgUfcgucuscsc | 2635 | antisense | 23 |
| GACGACAGUGGACUUGCUGCA | 1540 | gsascgacAfgUfGfGfacuugcugcaL96 | 2636 | sense | 21 |
| UGCAGCAAGUCCACUGUCGUCUC | 1541 | usGfscagCfaAfGfuccaCfuGfucgucsusc | 2637 | antisense | 23 |
| UUGGAGACGACAGUGGACUUG | 1542 | ususggagAfcGfAfCfaguggacuugL96 | 2638 | sense | 21 |
| CAAGUCCACUGUCGUCUCCAAAA | 1543 | csAfsaguCfcAfCfugucGfuCfuccaasasa | 2639 | antisense | 23 |
| UUUGGAGACGACAGUGGACUU | 1544 | ususuggaGfaCfGfAfcaguggacuuL96 | 2640 | sense | 21 |
| AAGUCCACUGUCGUCUCCAAAAU | 1545 | asAfsgucCfaCfUfgucgUfcUfccaaasasu | 2641 | antisense | 23 |
| GGCCACCUCCUCAAUUGAAGA | 1546 | gsgsccacCfuCfCfUfcaauugaagaL96 | 2642 | sense | 21 |
| UCUUCAAUUGAGGAGGUGGCCCA | 1547 | usCfsuucAfaUfUfgaggAfgGfuggccscsa | 2643 | antisense | 23 |
| GCCACCUCCUCAAUUGAAGAA | 1548 | gscscaccUfcCfUfCfaauugaagaaL96 | 2644 | sense | 21 |
| UUCUUCAAUUGAGGAGGUGGCCC | 1549 | usUfscuuCfaAfUfugagGfaGfguggcscsc | 2645 | antisense | 23 |
| CCUGGGCCACCUCCUCAAUUG | 1550 | cscsugggCfcAfCfCfuccucaauugL96 | 2646 | sense | 21 |
| CAAUUGAGGAGGUGGCCCAGGAA | 1551 | csAfsauuGfaGfGfagguGfgCfccaggsasa | 2647 | antisense | 23 |
| UCCUGGGCCACCUCCUCAAUU | 1552 | uscscuggGfcCfAfCfcuccucaauuL96 | 2648 | sense | 21 |
| AAUUGAGGAGGUGGCCCAGGAAC | 1553 | asAfsuugAfgGfAfggugGfcCfcaggasasc | 2649 | antisense | 23 |
| UGUAUGUUACUUCUUAGAGAG | 1554 | usgsuaugUfuAfCfUfucuuagagagL96 | 2650 | sense | 21 |
| CUCUCUAAGAAGUAACAUACAUC | 1555 | csUfscucUfaAfGfaaguAfaCfauacasusc | 2651 | antisense | 23 |
| GUAUGUUACUUCUUAGAGAGA | 1556 | gsusauguUfaCfUfUfcuuagagagaL96 | 2652 | sense | 21 |
| UCUCUCUAAGAAGUAACAUACAU | 1557 | usCfsucuCfuAfAfgaagUfaAfcauacsasu | 2653 | antisense | 23 |
| AGGAUGUAUGUUACUUCUUAG | 1558 | asgsgaugUfaUfGfUfuacuucuuagL96 | 2654 | sense | 21 |
| CUAAGAAGUAACAUACAUCCUAA | 1559 | csUfsaagAfaGfUfaacaUfaCfauccusasa | 2655 | antisense | 23 |
| UAGGAUGUAUGUUACUUCUUA | 1560 | usasggauGfuAfUfGfuuacuucuuaL96 | 2656 | sense | 21 |
| UAAGAAGUAACAUACAUCCUAAA | 1561 | usAfsagaAfgUfAfacauAfcAfuccuasasa | 2657 | antisense | 23 |
| AAAUGUUUUAGGAUGUAUGUU | 1562 | asasauguUfuUfAfGfgauguauguuL96 | 2658 | sense | 21 |
| AACAUACAUCCUAAAACAUUUGG | 1563 | asAfscauAfcAfUfccuaAfaAfcauuusgsg | 2659 | antisense | 23 |
| AAUGUUUUAGGAUGUAUGUUA | 1564 | asasuguuUfuAfGfGfauguauguuaL96 | 2660 | sense | 21 |
| UAACAUACAUCCUAAAACAUUUG | 1565 | usAfsacaUfaCfAfuccuAfaAfacauususg | 2661 | antisense | 23 |
| AUCCAAAUGUUUUAGGAUGUA | 1566 | asusccaaAfuGfUfUfuuaggauguaL96 | 2662 | sense | 21 |
| UACAUCCUAAAACAUUUGGAUAU | 1567 | usAfscauCfcUfAfaaacAfuUfuggausasu | 2663 | antisense | 23 |
| UAUCCAAAUGUUUUAGGAUGU | 1568 | usasuccaAfaUfGfUfuuuaggauguL96 | 2664 | sense | 21 |
| ACAUCCUAAAACAUUUGGAUAUA | 1569 | asCfsaucCfuAfAfaacaUfuUfggauasusa | 2665 | antisense | 23 |
| AUGGGUGGCGGUAAUUGGUGA | 1570 | asusggguGfgCfGfGfuaauuggugaL96 | 2666 | sense | 21 |
| UCACCAAUUACCGCCACCCAUUC | 1571 | usCfsaccAfaUfUfaccgCfcAfcccaususc | 2667 | antisense | 23 |
| UGGGUGGCGGUAAUUGGUGAU | 1572 | usgsggugGfcGfGfUfaauuggugauL96 | 2668 | sense | 21 |
| AUCACCAAUUACCGCCACCCAUU | 1573 | asUfscacCfaAfUfuaccGfcCfacccasusu | 2669 | antisense | 23 |
| UGGAAUGGGUGGCGGUAAUUG | 1574 | usgsgaauGfgGfUfGfgcgguaauugL96 | 2670 | sense | 21 |
| CAAUUACCGCCACCCAUUCCAAU | 1575 | csAfsauuAfcCfGfccacCfcAfuuccasasu | 2671 | antisense | 23 |
| UUGGAAUGGGUGGCGGUAAUU | 1576 | ususggaaUfgGfGfUfggcgguaauuL96 | 2672 | sense | 21 |
| AAUUACCGCCACCCAUUCCAAUU | 1577 | asAfsuuaCfcGfCfcaccCfaUfuccaasusu | 2673 | antisense | 23 |
| UUCAAAGUGUUGGUAAUGCCU | 1578 | ususcaaaGfuGfUfUfgguaaugccuL96 | 2674 | sense | 21 |
| AGGCAUUACCAACACUUUGAACC | 1579 | asGfsgcaUfuAfCfcaacAfcUfuugaascsc | 2675 | antisense | 23 |
| UCAAAGUGUUGGUAAUGCCUG | 1580 | uscsaaagUfgUfUfGfguaaugccugL96 | 2676 | sense | 21 |
| CAGGCAUUACCAACACUUUGAAC | 1581 | csAfsggcAfuUfAfccaaCfaCfuuugasasc | 2677 | antisense | 23 |
| CAGGUUCAAAGUGUUGGUAAU | 1582 | csasgguuCfaAfAfGfuguugguaauL96 | 2678 | sense | 21 |
| AUUACCAACACUUUGAACCUGAG | 1583 | asUfsuacCfaAfCfacuuUfgAfaccugsasg | 2679 | antisense | 23 |
| UCAGGUUCAAAGUGUUGGUAA | 1584 | uscsagguUfcAfAfAfguguugguaaL96 | 2680 | sense | 21 |
| UUACCAACACUUUGAACCUGAGC | 1585 | usUfsaccAfaCfAfcuuuGfaAfccugasgsc | 2681 | antisense | 23 |
| CCACCUCCUCAAUUGAAGAAG | 1586 | cscsaccuCfcUfCfAfauugaagaagL96 | 2682 | sense | 21 |
| CUUCUUCAAUUGAGGAGGUGGCC | 1587 | csUfsucuUfcAfAfuugaGfgAfgguggscsc | 2683 | antisense | 23 |
| CACCUCCUCAAUUGAAGAAGU | 1588 | csasccucCfuCfAfAfuugaagaaguL96 | 2684 | sense | 21 |
| ACUUCUUCAAUUGAGGAGGUGGC | 1589 | asCfsuucUfuCfAfauugAfgGfaggugsgsc | 2685 | antisense | 23 |
| UGGGCCACCUCCUCAAUUGAA | 1590 | usgsggccAfcCfUfCfcucaauugaaL96 | 2686 | sense | 21 |
| UUCAAUUGAGGAGGUGGCCCAGG | 1591 | usUfscaaUfuGfAfggagGfuGfgcccasgsg | 2687 | antisense | 23 |
| CUGGGCCACCUCCUCAAUUGA | 1592 | csusgggcCfaCfCfUfccucaauugaL96 | 2688 | sense | 21 |
| UCAAUUGAGGAGGUGGCCCAGGA | 1593 | usCfsaauUfgAfGfgaggUfgGfcccagsgsa | 2689 | antisense | 23 |
| GAGUGGGUGCCAGAAUGUGAA | 1594 | gsasguggGfuGfCfCfagaaugugaaL96 | 2690 | sense | 21 |
| UUCACAUUCUGGCACCCACUCAG | 1595 | usUfscacAfuUfCfuggcAfcCfcacucsasg | 2691 | antisense | 23 |
| AGUGGGUGCCAGAAUGUGAAA | 1596 | asgsugggUfgCfCfAfgaaugugaaaL96 | 2692 | sense | 21 |
| UUUCACAUUCUGGCACCCACUCA | 1597 | usUfsucaCfaUfUfcuggCfaCfccacuscsa | 2693 | antisense | 23 |
| CUCUGAGUGGGUGCCAGAAUG | 1598 | csuscugaGfuGfGfGfugccagaaugL96 | 2694 | sense | 21 |
| CAUUCUGGCACCCACUCAGAGCC | 1599 | csAfsuucUfgGfCfacccAfcUfcagagscsc | 2695 | antisense | 23 |
| GCUCUGAGUGGGUGCCAGAAU | 1600 | gscsucugAfgUfGfGfgugccagaauL96 | 2696 | sense | 21 |
| AUUCUGGCACCCACUCAGAGCCA | 1601 | asUfsucuGfgCfAfcccaCfuCfagagescsa | 2697 | antisense | 23 |
| GCACUGAUGUUCUGAAAGCUC | 1602 | gscsacugAfuGfUfUfcugaaagcucL96 | 2698 | sense | 21 |
| GAGCUUUCAGAACAUCAGUGCCU | 1603 | gsAfsgcuUfuCfAfgaacAfuCfagugcscsu | 2699 | antisense | 23 |
| CACUGAUGUUCUGAAAGCUCU | 1604 | csascugaUfgUfUfCfugaaagcucuL96 | 2700 | sense | 21 |
| AGAGCUUUCAGAACAUCAGUGCC | 1605 | asGfsagcUfuUfCfagaaCfaUfcagugscsc | 2701 | antisense | 23 |
| AAAGGCACUGAUGUUCUGAAA | 1606 | asasaggcAfcUfGfAfuguucugaaaL96 | 2702 | sense | 21 |
| UUUCAGAACAUCAGUGCCUUUCC | 1607 | usUfsucaGfaAfCfaucaGfuGfccuuuscsc | 2703 | antisense | 23 |
| GAAAGGCACUGAUGUUCUGAA | 1608 | gsasaaggCfaCfUfGfauguucugaaL96 | 2704 | sense | 21 |
| UUCAGAACAUCAGUGCCUUUCCG | 1609 | usUfscagAfaCfAfucagUfgCfcuuucscsg | 2705 | antisense | 23 |
| GGGAAGGUGGAAGUCUUCCUG | 1610 | gsgsgaagGfuGfGfAfagucuuccugL96 | 2706 | sense | 21 |
| CAGGAAGACUUCCACCUUCCCUU | 1611 | csAfsggaAfgAfCfuuccAfcCfuucccsusu | 2707 | antisense | 23 |
| GGAAGGUGGAAGUCUUCCUGG | 1612 | gsgsaaggUfgGfAfAfgucuuccuggL96 | 2708 | sense | 21 |
| CCAGGAAGACUUCCACCUUCCCU | 1613 | csCfsaggAfaGfAfcuucCfaCfcuuccscsu | 2709 | antisense | 23 |
| GGAAGGGAAGGUGGAAGUCUU | 1614 | gsgsaaggGfaAfGfGfuggaagucuuL96 | 2710 | sense | 21 |
| AAGACUUCCACCUUCCCUUCCAC | 1615 | asAfsgacUfuCfCfaccuUfcCfcuuccsasc | 2711 | antisense | 23 |
| UGGAAGGGAAGGUGGAAGUCU | 1616 | usgsgaagGfgAfAfGfguggaagucuL96 | 2712 | sense | 21 |
| AGACUUCCACCUUCCCUUCCACA | 1617 | asGfsacuUfcCfAfccuuCfcCfuuccascsa | 2713 | antisense | 23 |
| UGCUAAAUCAGUACUUCCAAA | 1618 | usgscuaaAfuCfAfGfuacuuccaaaL96 | 2714 | sense | 21 |
| UUUGGAAGUACUGAUUUAGCAUG | 1619 | usUfsuggAfaGfUfacugAfuUfuagcasusg | 2715 | antisense | 23 |
| GCUAAAUCAGUACUUCCAAAG | 1620 | gscsuaaaUfcAfGfUfacuuccaaagL96 | 2716 | sense | 21 |
| CUUUGGAAGUACUGAUUUAGCAU | 1621 | csUfsuugGfaAfGfuacuGfaUfuuagcsasu | 2717 | antisense | 23 |
| AACAUGCUAAAUCAGUACUUC | 1622 | asascaugCfuAfAfAfucaguacuucL96 | 2718 | sense | 21 |
| GAAGUACUGAUUUAGCAUGUUGU | 1623 | gsAfsaguAfcUfGfauuuAfgCfauguusgsu | 2719 | antisense | 23 |
| CAACAUGCUAAAUCAGUACUU | 1624 | csasacauGfcUfAfAfaucaguacuuL96 | 2720 | sense | 21 |
| AAGUACUGAUUUAGCAUGUUGUU | 1625 | asAfsguaCfuGfAfuuuaGfcAfuguugsusu | 2721 | antisense | 23 |
| CCACAACUCAGGAUGAAAAAU | 1626 | cscsacaaCfuCfAfGfgaugaaaaauL96 | 2722 | sense | 21 |
| AUUUUUCAUCCUGAGUUGUGGCG | 1627 | asUfsuuuUfcAfUfccugAfgUfuguggscsg | 2723 | antisense | 23 |
| CACAACUCAGGAUGAAAAAUU | 1628 | csascaacUfcAfGfGfaugaaaaauuL96 | 2724 | sense | 21 |
| AAUUUUUCAUCCUGAGUUGUGGC | 1629 | asAfsuuuUfuCfAfuccuGfaGfuugugsgsc | 2725 | antisense | 23 |
| GCCGCCACAACUCAGGAUGAA | 1630 | gscscgccAfcAfAfCfucaggaugaaL96 | 2726 | sense | 21 |
| UUCAUCCUGAGUUGUGGCGGCAG | 1631 | usUfscauCfcUfGfaguuGfuGfgcggcsasg | 2727 | antisense | 23 |
| UGCCGCCACAACUCAGGAUGA | 1632 | usgsccgcCfaCfAfAfcucaggaugaL96 | 2728 | sense | 21 |
| UCAUCCUGAGUUGUGGCGGCAGU | 1633 | usCfsaucCfuGfAfguugUfgGfcggcasgsu | 2729 | antisense | 23 |
| GCAACCGUCUGGAUGAUGUGC | 1634 | gscsaaccGfuCfUfGfgaugaugugcL96 | 2730 | sense | 21 |
| GCACAUCAUCCAGACGGUUGCCC | 1635 | gsCfsacaUfcAfUfccagAfcGfguugcscsc | 2731 | antisense | 23 |
| CAACCGUCUGGAUGAUGUGCG | 1636 | csasaccgUfcUfGfGfaugaugugcgL96 | 2732 | sense | 21 |
| CGCACAUCAUCCAGACGGUUGCC | 1637 | csGfscacAfuCfAfuccaGfaCfgguugscsc | 2733 | antisense | 23 |
| CUGGGCAACCGUCUGGAUGAU | 1638 | csusgggcAfaCfCfGfucuggaugauL96 | 2734 | sense | 21 |
| AUCAUCCAGACGGUUGCCCAGGU | 1639 | asUfscauCfcAfGfacggUfuGfcccagsgsu | 2735 | antisense | 23 |
| CCUGGGCAACCGUCUGGAUGA | 1640 | cscsugggCfaAfCfCfgucuggaugaL96 | 2736 | sense | 21 |
| UCAUCCAGACGGUUGCCCAGGUA | 1641 | usCfsaucCfaGfAfcgguUfgCfccaggsusa | 2737 | antisense | 23 |
| GCAAAUGAUGAAGAAACUUUG | 1642 | gscsaaauGfaUfGfAfagaaacuuugL96 | 2738 | sense | 21 |
| CAAAGUUUCUUCAUCAUUUGCCC | 1643 | csAfsaagUfuUfCfuucaUfcAfuuugcscsc | 2739 | antisense | 23 |
| CAAAUGAUGAAGAAACUUUGG | 1644 | csasaaugAfuGfAfAfgaaacuuuggL96 | 2740 | sense | 21 |
| CCAAAGUUUCUUCAUCAUUUGCC | 1645 | csCfsaaaGfuUfUfcuucAfuCfauuugscsc | 2741 | antisense | 23 |
| UGGGGCAAAUGAUGAAGAAAC | 1646 | usgsgggcAfaAfUfGfaugaagaaacL96 | 2742 | sense | 21 |
| GUUUCUUCAUCAUUUGCCCCAGA | 1647 | gsUfsuucUfuCfAfucauUfuGfccccasgsa | 2743 | antisense | 23 |
| CUGGGGCAAAUGAUGAAGAAA | 1648 | csusggggCfaAfAfUfgaugaagaaaL96 | 2744 | sense | 21 |
| UUUCUUCAUCAUUUGCCCCAGAC | 1649 | usUfsucuUfcAfUfcauuUfgCfcccagsasc | 2745 | antisense | 23 |
| CCAAGGCUGUGUUUGUGGGGA | 1650 | cscsaaggCfuGfUfGfuuuguggggaL96 | 2746 | sense | 21 |
| UCCCCACAAACACAGCCUUGGCG | 1651 | usCfscccAfcAfAfacacAfgCfcuuggscsg | 2747 | antisense | 23 |
| CAAGGCUGUGUUUGUGGGGAG | 1652 | csasaggcUfgUfGfUfuuguggggagL96 | 2748 | sense | 21 |
| CUCCCCACAAACACAGCCUUGGC | 1653 | csUfscccCfaCfAfaacaCfaGfccuugsgsc | 2749 | antisense | 23 |
| GGCGCCAAGGCUGUGUUUGUG | 1654 | gsgscgccAfaGfGfCfuguguuugugL96 | 2750 | sense | 21 |
| CACAAACACAGCCUUGGCGCCAA | 1655 | csAfscaaAfcAfCfagccUfuGfgcgccsasa | 2751 | antisense | 23 |
| UGGCGCCAAGGCUGUGUUUGU | 1656 | usgsgcgcCfaAfGfGfcuguguuuguL96 | 2752 | sense | 21 |
| ACAAACACAGCCUUGGCGCCAAG | 1657 | asCfsaaaCfaCfAfgccuUfgGfcgccasasg | 2753 | antisense | 23 |
| ACUGCCGCCACAACUCAGGAU | 1658 | ascsugccGfcCfAfCfaacucaggauL96 | 2754 | sense | 21 |
| AUCCUGAGUUGUGGCGGCAGUUU | 1659 | asUfsccuGfaGfUfugugGfcGfgcagususu | 2755 | antisense | 23 |
| CUGCCGCCACAACUCAGGAUG | 1660 | csusgccgCfcAfCfAfacucaggaugL96 | 2756 | sense | 21 |
| CAUCCUGAGUUGUGGCGGCAGUU | 1661 | csAfsuccUfgAfGfuuguGfgCfggcagsusu | 2757 | antisense | 23 |
| UCAAACUGCCGCCACAACUCA | 1662 | uscsaaacUfgCfCfGfccacaacucaL96 | 2758 | sense | 21 |
| UGAGUUGUGGCGGCAGUUUGAAU | 1663 | usGfsaguUfgUfGfgcggCfaGfuuugasasu | 2759 | antisense | 23 |
| UUCAAACUGCCGCCACAACUC | 1664 | ususcaaaCfuGfCfCfgccacaacucL96 | 2760 | sense | 21 |
| GAGUUGUGGCGGCAGUUUGAAUC | 1665 | gsAfsguuGfuGfGfcggcAfgUfuugaasusc | 2761 | antisense | 23 |
| GGGAAGAUAUCAAAUGGCUGA | 1666 | gsgsgaagAfuAfUfCfaaauggcugaL96 | 2762 | sense | 21 |
| UCAGCCAUUUGAUAUCUUCCCAG | 1667 | usCfsagcCfaUfUfugauAfuCfuucccsasg | 2763 | antisense | 23 |
| GGAAGAUAUCAAAUGGCUGAG | 1668 | gsgsaagaUfaUfCfAfaauggcugagL96 | 2764 | sense | 21 |
| CUCAGCCAUUUGAUAUCUUCCCA | 1669 | csUfscagCfcAfUfuugaUfaUfcuuccscsa | 2765 | antisense | 23 |
| AGCUGGGAAGAUAUCAAAUGG | 1670 | asgscuggGfaAfGfAfuaucaaauggL96 | 2766 | sense | 21 |
| CCAUUUGAUAUCUUCCCAGCUGA | 1671 | csCfsauuUfgAfUfaucuUfcCfcagcusgsa | 2767 | antisense | 23 |
| CAGCUGGGAAGAUAUCAAAUG | 1672 | csasgcugGfgAfAfGfauaucaaaugL96 | 2768 | sense | 21 |
| CAUUUGAUAUCUUCCCAGCUGAU | 1673 | csAfsuuuGfaUfAfucuuCfcCfagcugsasu | 2769 | antisense | 23 |
| AAUCAGUACUUCCAAAGUCUA | 1674 | asasucagUfaCfUfUfccaaagucuaL96 | 2770 | sense | 21 |
| UAGACUUUGGAAGUACUGAUUUA | 1675 | usAfsgacUfuUfGfgaagUfaCfugauususa | 2771 | antisense | 23 |
| AUCAGUACUUCCAAAGUCUAU | 1676 | asuscaguAfcUfUfCfcaaagucuauL96 | 2772 | sense | 21 |
| AUAGACUUUGGAAGUACUGAUUU | 1677 | asUfsagaCfuUfUfggaaGfuAfcugaususu | 2773 | antisense | 23 |
| GCUAAAUCAGUACUUCCAAAG | 1678 | gscsuaaaUfcAfGfUfacuuccaaagL96 | 2774 | sense | 21 |
| CUUUGGAAGUACUGAUUUAGCAU | 1679 | csUfsuugGfaAfGfuacuGfaUfuuagcsasu | 2775 | antisense | 23 |
| UGCUAAAUCAGUACUUCCAAA | 1680 | usgscuaaAfuCfAfGfuacuuccaaaL96 | 2776 | sense | 21 |
| UUUGGAAGUACUGAUUUAGCAUG | 1681 | usUfsuggAfaGfUfacugAfuUfuagcasusg | 2777 | antisense | 23 |
| UCAGCAUGCCAAUAUGUGUGG | 1682 | uscsagcaUfgCfCfAfauauguguggL96 | 2778 | sense | 21 |
| CCACACAUAUUGGCAUGCUGACC | 1683 | csCfsacaCfaUfAfuuggCfaUfgcugascsc | 2779 | antisense | 23 |
| CAGCAUGCCAAUAUGUGUGGG | 1684 | csasgcauGfcCfAfAfuaugugugggL96 | 2780 | sense | 21 |
| CCCACACAUAUUGGCAUGCUGAC | 1685 | csCfscacAfcAfUfauugGfcAfugcugsasc | 2781 | antisense | 23 |
| AGGGUCAGCAUGCCAAUAUGU | 1686 | asgsggucAfgCfAfUfgccaauauguL96 | 2782 | sense | 21 |
| ACAUAUUGGCAUGCUGACCCUCU | 1687 | asCfsauaUfuGfGfcaugCfuGfacccuscsu | 2783 | antisense | 23 |
| GAGGGUCAGCAUGCCAAUAUG | 1688 | gsasggguCfaGfCfAfugccaauaugL96 | 2784 | sense | 21 |
| CAUAUUGGCAUGCUGACCCUCUG | 1689 | csAfsuauUfgGfCfaugcUfgAfcccucsusg | 2785 | antisense | 23 |
| GCAUAUGUGGCUAAAGCAAUA | 1690 | gscsauauGfuGfGfCfuaaagcaauaL96 | 2786 | sense | 21 |
| UAUUGCUUUAGCCACAUAUGCAG | 1691 | usAfsuugCfuUfUfagccAfcAfuaugcsasg | 2787 | antisense | 23 |
| CAUAUGUGGCUAAAGCAAUAG | 1692 | csasuaugUfgGfCfUfaaagcaauagL96 | 2788 | sense | 21 |
| CUAUUGCUUUAGCCACAUAUGCA | 1693 | csUfsauuGfcUfUfuagcCfaCfauaugscsa | 2789 | antisense | 23 |
| UGCUGCAUAUGUGGCUAAAGC | 1694 | usgscugcAfuAfUfGfuggcuaaagcL96 | 2790 | sense | 21 |
| GCUUUAGCCACAUAUGCAGCAAG | 1695 | gsCfsuuuAfgCfCfacauAfuGfcagcasasg | 2791 | antisense | 23 |
| UUGCUGCAUAUGUGGCUAAAG | 1696 | ususgcugCfaUfAfUfguggcuaaagL96 | 2792 | sense | 21 |
| CUUUAGCCACAUAUGCAGCAAGU | 1697 | csUfsuuaGfcCfAfcauaUfgCfagcaasgsu | 2793 | antisense | 23 |
| AAAUGAUGAAGAAACUUUGGC | 1698 | asasaugaUfgAfAfGfaaacuuuggcL96 | 2794 | sense | 21 |
| GCCAAAGUUUCUUCAUCAUUUGC | 1699 | gsCfscaaAfgUfUfucuuCfaUfcauuusgsc | 2795 | antisense | 23 |
| AAUGAUGAAGAAACUUUGGCU | 1700 | asasugauGfaAfGfAfaacuuuggcuL96 | 2796 | sense | 21 |
| AGCCAAAGUUUCUUCAUCAUUUG | 1701 | asGfsccaAfaGfUfuucuUfcAfucauususg | 2797 | antisense | 23 |
| GGGCAAAUGAUGAAGAAACUU | 1702 | gsgsgcaaAfuGfAfUfgaagaaacuuL96 | 2798 | sense | 21 |
| AAGUUUCUUCAUCAUUUGCCCCA | 1703 | asAfsguuUfcUfUfcaucAfuUfugcccscsa | 2799 | antisense | 23 |
| GGGGCAAAUGAUGAAGAAACU | 1704 | gsgsggcaAfaUfGfAfugaagaaacuL96 | 2800 | sense | 21 |
| AGUUUCUUCAUCAUUUGCCCCAG | 1705 | asGfsuuuCfuUfCfaucaUfuUfgccccsasg | 2801 | antisense | 23 |
| GAGAUACUAAAGGAAGAAUUC | 1706 | gsasgauaCfuAfAfAfggaagaauucL96 | 2802 | sense | 21 |
| GAAUUCUUCCUUUAGUAUCUCGA | 1707 | gsAfsauuCfuUfCfcuuuAfgUfaucucsgsa | 2803 | antisense | 23 |
| AGAUACUAAAGGAAGAAUUCC | 1708 | asgsauacUfaAfAfGfgaagaauuccL96 | 2804 | sense | 21 |
| GGAAUUCUUCCUUUAGUAUCUCG | 1709 | gsGfsaauUfcUfUfccuuUfaGfuaucuscsg | 2805 | antisense | 23 |
| CCUCGAGAUACUAAAGGAAGA | 1710 | cscsucgaGfaUfAfCfuaaaggaagaL96 | 2806 | sense | 21 |
| UCUUCCUUUAGUAUCUCGAGGAC | 1711 | usCfsuucCfuUfUfaguaUfcUfcgaggsasc | 2807 | antisense | 23 |
| UCCUCGAGAUACUAAAGGAAG | 1712 | uscscucgAfgAfUfAfcuaaaggaagL96 | 2808 | sense | 21 |
| CUUCCUUUAGUAUCUCGAGGACA | 1713 | csUfsuccUfuUfAfguauCfuCfgaggascsa | 2809 | antisense | 23 |
| ACAACUCAGGAUGAAAAAUUU | 1714 | ascsaacuCfaGfGfAfugaaaaauuuL96 | 2810 | sense | 21 |
| AAAUUUUUCAUCCUGAGUUGUGG | 1715 | asAfsauuUfuUfCfauccUfgAfguugusgsg | 2811 | antisense | 23 |
| CAACUCAGGAUGAAAAAUUUU | 1716 | csasacucAfgGfAfUfgaaaaauuuuL96 | 2812 | sense | 21 |
| AAAAUUUUUCAUCCUGAGUUGUG | 1717 | asAfsaauUfuUfUfcaucCfuGfaguugsusg | 2813 | antisense | 23 |
| CGCCACAACUCAGGAUGAAAA | 1718 | csgsccacAfaCfUfCfaggaugaaaaL96 | 2814 | sense | 21 |
| UUUUCAUCCUGAGUUGUGGCGGC | 1719 | usUfsuucAfuCfCfugagUfuGfuggcgsgsc | 2815 | antisense | 23 |
| CCGCCACAACUCAGGAUGAAA | 1720 | cscsgccaCfaAfCfUfcaggaugaaaL96 | 2816 | sense | 21 |
| UUUCAUCCUGAGUUGUGGCGGCA | 1721 | usUfsucaUfcCfUfgaguUfgUfggcggscsa | 2817 | antisense | 23 |
| AGGGAAGGUGGAAGUCUUCCU | 1722 | asgsggaaGfgUfGfGfaagucuuccuL96 | 2818 | sense | 21 |
| AGGAAGACUUCCACCUUCCCUUC | 1723 | asGfsgaaGfaCfUfuccaCfcUfucccususc | 2819 | antisense | 23 |
| GGGAAGGUGGAAGUCUUCCUG | 1724 | gsgsgaagGfuGfGfAfagucuuccugL96 | 2820 | sense | 21 |
| CAGGAAGACUUCCACCUUCCCUU | 1725 | csAfsggaAfgAfCfuuccAfcCfuucccsusu | 2821 | antisense | 23 |
| UGGAAGGGAAGGUGGAAGUCU | 1726 | usgsgaagGfgAfAfGfguggaagucuL96 | 2822 | sense | 21 |
| AGACUUCCACCUUCCCUUCCACA | 1727 | asGfsacuUfcCfAfccuuCfcCfuuccascsa | 2823 | antisense | 23 |
| GUGGAAGGGAAGGUGGAAGUC | 1728 | gsusggaaGfgGfAfAfgguggaagucL96 | 2824 | sense | 21 |
| GACUUCCACCUUCCCUUCCACAG | 1729 | gsAfscuuCfcAfCfcuucCfcUfuccacsasg | 2825 | antisense | 23 |
| GGCGAGCUUGCCACUGUGAGA | 1730 | gsgscgagCfuUfGfCfcacugugagaL96 | 2826 | sense | 21 |
| UCUCACAGUGGCAAGCUCGCCGU | 1731 | usCfsucaCfaGfUfggcaAfgCfucgccsgsu | 2827 | antisense | 23 |
| GCGAGCUUGCCACUGUGAGAG | 1732 | gscsgagcUfuGfCfCfacugugagagL96 | 2828 | sense | 21 |
| CUCUCACAGUGGCAAGCUCGCCG | 1733 | csUfscucAfcAfGfuggcAfaGfcucgcscsg | 2829 | antisense | 23 |
| GGACGGCGAGCUUGCCACUGU | 1734 | gsgsacggCfgAfGfCfuugccacuguL96 | 2830 | sense | 21 |
| ACAGUGGCAAGCUCGCCGUCCAC | 1735 | asCfsaguGfgCfAfagcuCfgCfcguccsasc | 2831 | antisense | 23 |
| UGGACGGCGAGCUUGCCACUG | 1736 | usgsgacgGfcGfAfGfcuugccacugL96 | 2832 | sense | 21 |
| CAGUGGCAAGCUCGCCGUCCACA | 1737 | csAfsgugGfcAfAfgcucGfcCfguccascsa | 2833 | antisense | 23 |
| AUGUGCGUAACAGAUUCAAAC | 1738 | asusgugcGfuAfAfCfagauucaaacL96 | 2834 | sense | 21 |
| GUUUGAAUCUGUUACGCACAUCA | 1739 | gsUfsuugAfaUfCfuguuAfcGfcacauscsa | 2835 | antisense | 23 |
| UGUGCGUAACAGAUUCAAACU | 1740 | usgsugcgUfaAfCfAfgauucaaacuL96 | 2836 | sense | 21 |
| AGUUUGAAUCUGUUACGCACAUC | 1741 | asGfsuuuGfaAfUfcuguUfaCfgcacasusc | 2837 | antisense | 23 |
| GAUGAUGUGCGUAACAGAUUC | 1742 | gsasugauGfuGfCfGfuaacagauucL96 | 2838 | sense | 21 |
| GAAUCUGUUACGCACAUCAUCCA | 1743 | gsAfsaucUfgUfUfacgcAfcAfucaucscsa | 2839 | antisense | 23 |
| GGAUGAUGUGCGUAACAGAUU | 1744 | gsgsaugaUfgUfGfCfguaacagauuL96 | 2840 | sense | 21 |
| AAUCUGUUACGCACAUCAUCCAG | 1745 | asAfsucuGfuUfAfcgcaCfaUfcauccsasg | 2841 | antisense | 23 |
| GGGUCAGCAUGCCAAUAUGUG | 1746 | gsgsgucaGfcAfUfGfccaauaugugL96 | 2842 | sense | 21 |
| CACAUAUUGGCAUGCUGACCCUC | 1747 | csAfscauAfuUfGfgcauGfcUfgacccsusc | 2843 | antisense | 23 |
| GGUCAGCAUGCCAAUAUGUGU | 1748 | gsgsucagCfaUfGfCfcaauauguguL96 | 2844 | sense | 21 |
| ACACAUAUUGGCAUGCUGACCCU | 1749 | asCfsacaUfaUfUfggcaUfgCfugaccscsu | 2845 | antisense | 23 |
| CAGAGGGUCAGCAUGCCAAUA | 1750 | csasgaggGfuCfAfGfcaugccaauaL96 | 2846 | sense | 21 |
| UAUUGGCAUGCUGACCCUCUGUC | 1751 | usAfsuugGfcAfUfgcugAfcCfcucugsusc | 2847 | antisense | 23 |
| ACAGAGGGUCAGCAUGCCAAU | 1752 | ascsagagGfgUfCfAfgcaugccaauL96 | 2848 | sense | 21 |
| AUUGGCAUGCUGACCCUCUGUCC | 1753 | asUfsuggCfaUfGfcugaCfcCfucuguscsc | 2849 | antisense | 23 |
| GCUUGAAUGGGAUCUUGGUGU | 1754 | gscsuugaAfuGfGfGfaucuugguguL96 | 2850 | sense | 21 |
| ACACCAAGAUCCCAUUCAAGCCA | 1755 | asCfsaccAfaGfAfucccAfuUfcaagcscsa | 2851 | antisense | 23 |
| CUUGAAUGGGAUCUUGGUGUC | 1756 | csusugaaUfgGfGfAfucuuggugucL96 | 2852 | sense | 21 |
| GACACCAAGAUCCCAUUCAAGCC | 1757 | gsAfscacCfaAfGfauccCfaUfucaagscsc | 2853 | antisense | 23 |
| CAUGGCUUGAAUGGGAUCUUG | 1758 | csasuggcUfuGfAfAfugggaucuugL96 | 2854 | sense | 21 |
| CAAGAUCCCAUUCAAGCCAUGUU | 1759 | csAfsagaUfcCfCfauucAfaGfccaugsusu | 2855 | antisense | 23 |
| ACAUGGCUUGAAUGGGAUCUU | 1760 | ascsauggCfuUfGfAfaugggaucuuL96 | 2856 | sense | 21 |
| AAGAUCCCAUUCAAGCCAUGUUU | 1761 | asAfsgauCfcCfAfuucaAfgCfcaugususu | 2857 | antisense | 23 |
| UCAAAUGGCUGAGAAGACUGA | 1762 | uscsaaauGfgCfUfGfagaagacugaL96 | 2858 | sense | 21 |
| UCAGUCUUCUCAGCCAUUUGAUA | 1763 | usCfsaguCfuUfCfucagCfcAfuuugasusa | 2859 | antisense | 23 |
| CAAAUGGCUGAGAAGACUGAC | 1764 | csasaaugGfcUfGfAfgaagacugacL96 | 2860 | sense | 21 |
| GUCAGUCUUCUCAGCCAUUUGAU | 1765 | gsUfscagUfcUfUfcucaGfcCfauuugsasu | 2861 | antisense | 23 |
| GAUAUCAAAUGGCUGAGAAGA | 1766 | gsasuaucAfaAfUfGfgcugagaagaL96 | 2862 | sense | 21 |
| UCUUCUCAGCCAUUUGAUAUCUU | 1767 | usCfsuucUfcAfGfccauUfuGfauaucsusu | 2863 | antisense | 23 |
| AGAUAUCAAAUGGCUGAGAAG | 1768 | asgsauauCfaAfAfUfggcugagaagL96 | 2864 | sense | 21 |
| CUUCUCAGCCAUUUGAUAUCUUC | 1769 | csUfsucuCfaGfCfcauuUfgAfuaucususc | 2865 | antisense | 23 |
| GAAAGUCAUCGACAAGACAUU | 1770 | gsasaaguCfaUfCfGfacaagacauuL96 | 2866 | sense | 21 |
| AAUGUCUUGUCGAUGACUUUCAC | 1771 | asAfsuguCfuUfGfucgaUfgAfcuuucsasc | 2867 | antisense | 23 |
| AAAGUCAUCGACAAGACAUUG | 1772 | asasagucAfuCfGfAfcaagacauugL96 | 2868 | sense | 21 |
| CAAUGUCUUGUCGAUGACUUUCA | 1773 | csAfsaugUfcUfUfgucgAfuGfacuuuscsa | 2869 | antisense | 23 |
| AUGUGAAAGUCAUCGACAAGA | 1774 | asusgugaAfaGfUfCfaucgacaagaL96 | 2870 | sense | 21 |
| UCUUGUCGAUGACUUUCACAUUC | 1775 | usCfsuugUfcGfAfugacUfuUfcacaususc | 2871 | antisense | 23 |
| AAUGUGAAAGUCAUCGACAAG | 1776 | asasugugAfaAfGfUfcaucgacaagL96 | 2872 | sense | 21 |
| CUUGUCGAUGACUUUCACAUUCU | 1777 | csUfsuguCfgAfUfgacuUfuCfacauuscsu | 2873 | antisense | 23 |
| GGCUAAUUUGUAUCAAUGAUU | 1778 | gsgscuaaUfuUfGfUfaucaaugauuL96 | 2874 | sense | 21 |
| AAUCAUUGAUACAAAUUAGCCGG | 1779 | asAfsucaUfuGfAfuacaAfaUfuagccsgsg | 2875 | antisense | 23 |
| GCUAAUUUGUAUCAAUGAUUA | 1780 | gscsuaauUfuGfUfAfucaaugauuaL96 | 2876 | sense | 21 |
| UAAUCAUUGAUACAAAUUAGCCG | 1781 | usAfsaucAfuUfGfauacAfaAfuuagcscsg | 2877 | antisense | 23 |
| CCCCGGCUAAUUUGUAUCAAU | 1782 | cscsccggCfuAfAfUfuuguaucaauL96 | 2878 | sense | 21 |
| AUUGAUACAAAUUAGCCGGGGGA | 1783 | asUfsugaUfaCfAfaauuAfgCfcggggsgsa | 2879 | antisense | 23 |
| CCCCCGGCUAAUUUGUAUCAA | 1784 | cscscccgGfcUfAfAfuuuguaucaaL96 | 2880 | sense | 21 |
| UUGAUACAAAUUAGCCGGGGGAG | 1785 | usUfsgauAfcAfAfauuaGfcCfgggggsasg | 2881 | antisense | 23 |
| UGUCGACUUCUGUUUUAGGAC | 1786 | usgsucgaCfuUfCfUfguuuuaggacL96 | 2882 | sense | 21 |
| GUCCUAAAACAGAAGUCGACAGA | 1787 | gsUfsccuAfaAfAfcagaAfgUfcgacasgsa | 2883 | antisense | 23 |
| GUCGACUUCUGUUUUAGGACA | 1788 | gsuscgacUfuCfUfGfuuuuaggacaL96 | 2884 | sense | 21 |
| UGUCCUAAAACAGAAGUCGACAG | 1789 | usGfsuccUfaAfAfacagAfaGfucgacsasg | 2885 | antisense | 23 |
| GAUCUGUCGACUUCUGUUUUA | 1790 | gsasucugUfcGfAfCfuucuguuuuaL96 | 2886 | sense | 21 |
| UAAAACAGAAGUCGACAGAUCUG | 1791 | usAfsaaaCfaGfAfagucGfaCfagaucsusg | 2887 | antisense | 23 |
| AGAUCUGUCGACUUCUGUUUU | 1792 | asgsaucuGfuCfGfAfcuucuguuuuL96 | 2888 | sense | 21 |
| AAAACAGAAGUCGACAGAUCUGU | 1793 | asAfsaacAfgAfAfgucgAfcAfgaucusgsu | 2889 | antisense | 23 |
| CCGAGAAGUCACCAAGAAGCU | 1794 | cscsgagaAfgUfCfAfccaagaagcuL96 | 2890 | sense | 21 |
| AGCUUCUUGGUGACUUCUCGGUC | 1795 | asGfscuuCfuUfGfgugaCfuUfcucggsusc | 2891 | antisense | 23 |
| CGAGAAGUCACCAAGAAGCUA | 1796 | csgsagaaGfuCfAfCfcaagaagcuaL96 | 2892 | sense | 21 |
| UAGCUUCUUGGUGACUUCUCGGU | 1797 | usAfsgcuUfcUfUfggugAfcUfucucgsgsu | 2893 | antisense | 23 |
| AGGACCGAGAAGUCACCAAGA | 1798 | asgsgaccGfaGfAfAfgucaccaagaL96 | 2894 | sense | 21 |
| UCUUGGUGACUUCUCGGUCCUUG | 1799 | usCfsuugGfuGfAfcuucUfcGfguccususg | 2895 | antisense | 23 |
| AAGGACCGAGAAGUCACCAAG | 1800 | asasggacCfgAfGfAfagucaccaagL96 | 2896 | sense | 21 |
| CUUGGUGACUUCUCGGUCCUUGU | 1801 | csUfsuggUfgAfCfuucuCfgGfuccuusgsu | 2897 | antisense | 23 |
| AAACAUGGCUUGAAUGGGAUC | 1802 | asasacauGfgCfUfUfgaaugggaucL96 | 2898 | sense | 21 |
| GAUCCCAUUCAAGCCAUGUUUAA | 1803 | gsAfsuccCfaUfUfcaagCfcAfuguuusasa | 2899 | antisense | 23 |
| AACAUGGCUUGAAUGGGAUCU | 1804 | asascaugGfcUfUfGfaaugggaucuL96 | 2900 | sense | 21 |
| AGAUCCCAUUCAAGCCAUGUUUA | 1805 | asGfsaucCfcAfUfucaaGfcCfauguususa | 2901 | antisense | 23 |
| UGUUAAACAUGGCUUGAAUGG | 1806 | usgsuuaaAfcAfUfGfgcuugaauggL96 | 2902 | sense | 21 |
| CCAUUCAAGCCAUGUUUAACAGC | 1807 | csCfsauuCfaAfGfccauGfuUfuaacasgsc | 2903 | antisense | 23 |
| CUGUUAAACAUGGCUUGAAUG | 1808 | csusguuaAfaCfAfUfggcuugaaugL96 | 2904 | sense | 21 |
| CAUUCAAGCCAUGUUUAACAGCC | 1809 | csAfsuucAfaGfCfcaugUfuUfaacagscsc | 2905 | antisense | 23 |
| GACUUGCUGCAUAUGUGGCUA | 1810 | gsascuugCfuGfCfAfuauguggcuaL96 | 2906 | sense | 21 |
| UAGCCACAUAUGCAGCAAGUCCA | 1811 | usAfsgccAfcAfUfaugcAfgCfaagucscsa | 2907 | antisense | 23 |
| ACUUGCUGCAUAUGUGGCUAA | 1812 | ascsuugcUfgCfAfUfauguggcuaaL96 | 2908 | sense | 21 |
| UUAGCCACAUAUGCAGCAAGUCC | 1813 | usUfsagcCfaCfAfuaugCfaGfcaaguscsc | 2909 | antisense | 23 |
| AGUGGACUUGCUGCAUAUGUG | 1814 | asgsuggaCfuUfGfCfugcauaugugL96 | 2910 | sense | 21 |
| CACAUAUGCAGCAAGUCCACUGU | 1815 | csAfscauAfuGfCfagcaAfgUfccacusgsu | 2911 | antisense | 23 |
| CAGUGGACUUGCUGCAUAUGU | 1816 | csasguggAfcUfUfGfcugcauauguL96 | 2912 | sense | 21 |
| ACAUAUGCAGCAAGUCCACUGUC | 1817 | asCfsauaUfgCfAfgcaaGfuCfcacugsusc | 2913 | antisense | 23 |
| UAAAUCAGUACUUCCAAAGUC | 1818 | usasaaucAfgUfAfCfuuccaaagucL96 | 2914 | sense | 21 |
| GACUUUGGAAGUACUGAUUUAGC | 1819 | gsAfscuuUfgGfAfaguaCfuGfauuuasgsc | 2915 | antisense | 23 |
| AAAUCAGUACUUCCAAAGUCU | 1820 | asasaucaGfuAfCfUfuccaaagucuL96 | 2916 | sense | 21 |
| AGACUUUGGAAGUACUGAUUUAG | 1821 | asGfsacuUfuGfGfaaguAfcUfgauuusasg | 2917 | antisense | 23 |
| AUGCUAAAUCAGUACUUCCAA | 1822 | asusgcuaAfaUfCfAfguacuuccaaL96 | 2918 | sense | 21 |
| UUGGAAGUACUGAUUUAGCAUGU | 1823 | usUfsggaAfgUfAfcugaUfuUfagcausgsu | 2919 | antisense | 23 |
| CAUGCUAAAUCAGUACUUCCA | 1824 | csasugcuAfaAfUfCfaguacuuccaL96 | 2920 | sense | 21 |
| UGGAAGUACUGAUUUAGCAUGUU | 1825 | usGfsgaaGfuAfCfugauUfuAfgcaugsusu | 2921 | antisense | 23 |
| UCCUCAAUUGAAGAAGUGGCG | 1826 | uscscucaAfuUfGfAfagaaguggcgL96 | 2922 | sense | 21 |
| CGCCACUUCUUCAAUUGAGGAGG | 1827 | csGfsccaCfuUfCfuucaAfuUfgaggasgsg | 2923 | antisense | 23 |
| CCUCAAUUGAAGAAGUGGCGG | 1828 | cscsucaaUfuGfAfAfgaaguggcggL96 | 2924 | sense | 21 |
| CCGCCACUUCUUCAAUUGAGGAG | 1829 | csCfsgccAfcUfUfcuucAfaUfugaggsasg | 2925 | antisense | 23 |
| CACCUCCUCAAUUGAAGAAGU | 1830 | csasccucCfuCfAfAfuugaagaaguL96 | 2926 | sense | 21 |
| ACUUCUUCAAUUGAGGAGGUGGC | 1831 | asCfsuucUfuCfAfauugAfgGfaggugsgsc | 2927 | antisense | 23 |
| CCACCUCCUCAAUUGAAGAAG | 1832 | cscsaccuCfcUfCfAfauugaagaagL96 | 2928 | sense | 21 |
| CUUCUUCAAUUGAGGAGGUGGCC | 1833 | csUfsucuUfcAfAfuugaGfgAfgguggscsc | 2929 | antisense | 23 |
| CAAGAUGUCCUCGAGAUACUA | 1834 | csasagauGfuCfCfUfcgagauacuaL96 | 2930 | sense | 21 |
| UAGUAUCUCGAGGACAUCUUGAA | 1835 | usAfsguaUfcUfCfgaggAfcAfucuugsasa | 2931 | antisense | 23 |
| AAGAUGUCCUCGAGAUACUAA | 1836 | asasgaugUfcCfUfCfgagauacuaaL96 | 2932 | sense | 21 |
| UUAGUAUCUCGAGGACAUCUUGA | 1837 | usUfsaguAfuCfUfcgagGfaCfaucuusgsa | 2933 | antisense | 23 |
| UGUUCAAGAUGUCCUCGAGAU | 1838 | usgsuucaAfgAfUfGfuccucgagauL96 | 2934 | sense | 21 |
| AUCUCGAGGACAUCUUGAACACC | 1839 | asUfscucGfaGfGfacauCfuUfgaacascsc | 2935 | antisense | 23 |
| GUGUUCAAGAUGUCCUCGAGA | 1840 | gsusguucAfaGfAfUfguccucgagaL96 | 2936 | sense | 21 |
| UCUCGAGGACAUCUUGAACACCU | 1841 | usCfsucgAfgGfAfcaucUfuGfaacacscsu | 2937 | antisense | 23 |
| ACAUGCUAAAUCAGUACUUCC | 1842 | ascsaugcUfaAfAfUfcaguacuuccL96 | 2938 | sense | 21 |
| GGAAGUACUGAUUUAGCAUGUUG | 1843 | gsGfsaagUfaCfUfgauuUfaGfcaugususg | 2939 | antisense | 23 |
| CAUGCUAAAUCAGUACUUCCA | 1844 | csasugcuAfaAfUfCfaguacuuccaL96 | 2940 | sense | 21 |
| UGGAAGUACUGAUUUAGCAUGUU | 1845 | usGfsgaaGfuAfCfugauUfuAfgcaugsusu | 2941 | antisense | 23 |
| AACAACAUGCUAAAUCAGUAC | 1846 | asascaacAfuGfCfUfaaaucaguacL96 | 2942 | sense | 21 |
| GUACUGAUUUAGCAUGUUGUUCA | 1847 | gsUfsacuGfaUfUfuagcAfuGfuuguuscsa | 2943 | antisense | 23 |
| GAACAACAUGCUAAAUCAGUA | 1848 | gsasacaaCfaUfGfCfuaaaucaguaL96 | 2944 | sense | 21 |
| UACUGAUUUAGCAUGUUGUUCAU | 1849 | usAfscugAfuUfUfagcaUfgUfuguucsasu | 2945 | antisense | 23 |
| GAAAGGCACUGAUGUUCUGAA | 1850 | gsasaaggCfaCfUfGfauguucugaaL96 | 2946 | sense | 21 |
| UUCAGAACAUCAGUGCCUUUCCG | 1851 | usUfscagAfaCfAfucagUfgCfcuuucscsg | 2947 | antisense | 23 |
| AAAGGCACUGAUGUUCUGAAA | 1852 | asasaggcAfcUfGfAfuguucugaaaL96 | 2948 | sense | 21 |
| UUUCAGAACAUCAGUGCCUUUCC | 1853 | usUfsucaGfaAfCfaucaGfuGfccuuuscsc | 2949 | antisense | 23 |
| UGCGGAAAGGCACUGAUGUUC | 1854 | usgscggaAfaGfGfCfacugauguucL96 | 2950 | sense | 21 |
| GAACAUCAGUGCCUUUCCGCACA | 1855 | gsAfsacaUfcAfGfugccUfuUfccgcascsa | 2951 | antisense | 23 |
| GUGCGGAAAGGCACUGAUGUU | 1856 | gsusgcggAfaAfGfGfcacugauguuL96 | 2952 | sense | 21 |
| AACAUCAGUGCCUUUCCGCACAC | 1857 | asAfscauCfaGfUfgccuUfuCfcgcacsasc | 2953 | antisense | 23 |
| GUCAGCAUGCCAAUAUGUGUG | 1858 | gsuscagcAfuGfCfCfaauaugugugL96 | 2954 | sense | 21 |
| CACACAUAUUGGCAUGCUGACCC | 1859 | csAfscacAfuAfUfuggcAfuGfcugacscsc | 2955 | antisense | 23 |
| UCAGCAUGCCAAUAUGUGUGG | 1860 | uscsagcaUfgCfCfAfauauguguggL96 | 2956 | sense | 21 |
| CCACACAUAUUGGCAUGCUGACC | 1861 | csCfsacaCfaUfAfuuggCfaUfgcugascsc | 2957 | antisense | 23 |
| GAGGGUCAGCAUGCCAAUAUG | 1862 | gsasggguCfaGfCfAfugccaauaugL96 | 2958 | sense | 21 |
| CAUAUUGGCAUGCUGACCCUCUG | 1863 | csAfsuauUfgGfCfaugcUfgAfcccucsusg | 2959 | antisense | 23 |
| AGAGGGUCAGCAUGCCAAUAU | 1864 | asgsagggUfcAfGfCfaugccaauauL96 | 2960 | sense | 21 |
| AUAUUGGCAUGCUGACCCUCUGU | 1865 | asUfsauuGfgCfAfugcuGfaCfccucusgsu | 2961 | antisense | 23 |
| GAUGCUCCGGAAUGUUGCUGA | 1866 | gsasugcuCfcGfGfAfauguugcugaL96 | 2962 | sense | 21 |
| UCAGCAACAUUCCGGAGCAUCCU | 1867 | usCfsagcAfaCfAfuuccGfgAfgcaucscsu | 2963 | antisense | 23 |
| AUGCUCCGGAAUGUUGCUGAA | 1868 | asusgcucCfgGfAfAfuguugcugaaL96 | 2964 | sense | 21 |
| UUCAGCAACAUUCCGGAGCAUCC | 1869 | usUfscagCfaAfCfauucCfgGfagcauscsc | 2965 | antisense | 23 |
| CAAGGAUGCUCCGGAAUGUUG | 1870 | csasaggaUfgCfUfCfcggaauguugL96 | 2966 | sense | 21 |
| CAACAUUCCGGAGCAUCCUUGGA | 1871 | csAfsacaUfuCfCfggagCfaUfccuugsgsa | 2967 | antisense | 23 |
| CCAAGGAUGCUCCGGAAUGUU | 1872 | cscsaaggAfuGfCfUfccggaauguuL96 | 2968 | sense | 21 |
| AACAUUCCGGAGCAUCCUUGGAU | 1873 | asAfscauUfcCfGfgagcAfuCfcuuggsasu | 2969 | antisense | 23 |
| GCGUAACAGAUUCAAACUGCC | 1874 | gscsguaaCfaGfAfUfucaaacugccL96 | 2970 | sense | 21 |
| GGCAGUUUGAAUCUGUUACGCAC | 1875 | gsGfscagUfuUfGfaaucUfgUfuacgcsasc | 2971 | antisense | 23 |
| CGUAACAGAUUCAAACUGCCG | 1876 | csgsuaacAfgAfUfUfcaaacugccgL96 | 2972 | sense | 21 |
| CGGCAGUUUGAAUCUGUUACGCA | 1877 | csGfsgcaGfuUfUfgaauCfuGfuuacgscsa | 2973 | antisense | 23 |
| AUGUGCGUAACAGAUUCAAAC | 1878 | asusgugcGfuAfAfCfagauucaaacL96 | 2974 | sense | 21 |
| GUUUGAAUCUGUUACGCACAUCA | 1879 | gsUfsuugAfaUfCfuguuAfcGfcacauscsa | 2975 | antisense | 23 |
| GAUGUGCGUAACAGAUUCAAA | 1880 | gsasugugCfgUfAfAfcagauucaaaL96 | 2976 | sense | 21 |
| UUUGAAUCUGUUACGCACAUCAU | 1881 | usUfsugaAfuCfUfguuaCfgCfacaucsasu | 2977 | antisense | 23 |
| AGAGAAGAUGGGCUACAAGGC | 1882 | asgsagaaGfaUfGfGfgcuacaaggcL96 | 2978 | sense | 21 |
| GCCUUGUAGCCCAUCUUCUCUGC | 1883 | gsCfscuuGfuAfGfcccaUfcUfucucusgsc | 2979 | antisense | 23 |
| GAGAAGAUGGGCUACAAGGCC | 1884 | gsasgaagAfuGfGfGfcuacaaggccL96 | 2980 | sense | 21 |
| GGCCUUGUAGCCCAUCUUCUCUG | 1885 | gsGfsccuUfgUfAfgcccAfuCfuucucsusg | 2981 | antisense | 23 |
| AGGCAGAGAAGAUGGGCUACA | 1886 | asgsgcagAfgAfAfGfaugggcuacaL96 | 2982 | sense | 21 |
| UGUAGCCCAUCUUCUCUGCCUGC | 1887 | usGfsuagCfcCfAfucuuCfuCfugccusgsc | 2983 | antisense | 23 |
| CAGGCAGAGAAGAUGGGCUAC | 1888 | csasggcaGfaGfAfAfgaugggcuacL96 | 2984 | sense | 21 |
| GUAGCCCAUCUUCUCUGCCUGCC | 1889 | gsUfsagcCfcAfUfcuucUfcUfgccugscsc | 2985 | antisense | 23 |

TABLE 13
Modified antisense polynucleotides targeting HAO1.

| | | | SEQ |
|---|---|---|---|
| | Oligo | | ID |
| Target | Name | Sequence 5′-3′ | NO: |
| HAO1 | A-133284.1 | gsgsgsasgs(5MdC)sdAsdTsdTsdTsdTs(5MdC)sdAs(5MdC)sdAsgsgsususa | 4155 |
| HAO1 | A-133285.1 | asasususasdGs(5MdC)s(5MdC)sdGsdGsdGsdGsdGsdAsdGscsasususu | 4156 |
| HAO1 | A-133286.1 | asuscsasusdTsdGsdAsdTsdAs(5MdC)sdAsdAsdAsdTsusasgscsc | 4157 |
| HAO1 | A-133287.1 | gsususgsusdTs(5MdC)sdAsdTsdAsdAsdTs(5MdC)sdAsdTsusgsasusa | 4158 |
| HAO1 | A-133288.1 | gsasusususdAsdGs(5MdC)sdAsdTsdGsdTsdTsdGsdTsuscsasusa | 4159 |
| HAO1 | A-133289.1 | ususgsgsasdAsdGsdTsdAs(5MdC)sdTsdGsdAsdTsdTsusasgscsa | 4160 |
| HAO1 | A-133290.1 | csasusasusdAsdTsdAsdGsdAs(5MdC)sdTsdTsdTsdGsgsasasgsu | 4161 |
| HAO1 | A-133291.1 | csusgsusasdAsdTsdAsdGsdTs(5MdC)sdAsdTsdAsdTsasusasgsa | 4162 |
| HAO1 | A-133292.1 | ususgscscs(5MdC)s(5MdC)sdAsdGsdAs(5MdC)s(5MdC)sdTsdGsdTsasasusasg | 4163 |
| HAO1 | A-133293.1 | ususcsusus(5MdC)sdAsdTs(5MdC)sdAsdTsdTsdTsdGs(5MdC)scscscsasg | 4164 |
| HAO1 | A-133294.1 | usasuscsasdGs(5MdC)s(5MdC)sdAsdAsdAsdGsdTsdTsdTscsususcsa | 4165 |
| HAO1 | A-133295.1 | gscsusgscsdAsdAsdTsdAsdTsdTsdAsdTs(5MdC)sdAsgscscsasa | 4166 |
| HAO1 | A-133296.1 | asuscsusgsdGsdAsdAsdAsdAsdTsdGs(5MdC)sdTsdGscsasasusa | 4167 |
| HAO1 | A-133297.1 | gsasusascsdAsdGs(5MdC)sdTsdTs(5MdC)s(5MdC)sdAsdTs(5MdC)susgsgsasa | 4168 |
| HAO1 | A-133298.1 | gsasgscsasdTs(5MdC)s(5MdC)sdTsdTsdGsdGsdAsdTsdAscsasgscsu | 4169 |
| HAO1 | A-133299.1 | csasascsasdTsdTs(5MdC)s(5MdC)sdGsdGsdAsdGs(5MdC)sdAsuscscsusu | 4170 |
| HAO1 | A-133300.1 | gsasuscsusdGsdTsdTsdTs(5MdC)sdAsdGs(5MdC)sdAsdAscsasususc | 4171 |
| HAO1 | A-133301.1 | asgsasasgsdTs(5MdC)sdGsdAs(5MdC)sdAsdGsdAsdTs(5MdC)susgsususu | 4172 |
| HAO1 | A-133302.1 | usgsuscscsdTsdAsdAsdAsdAs(5MdC)sdAsdGsdAsdAsgsuscsgsa | 4173 |
| HAO1 | A-133303.1 | usgscsusgsdAs(5MdC)s(5MdC)s(5MdC)sdTs(5MdC)sdTsdGsdTs(5MdC)scsusasasa | 4174 |
| HAO1 | A-133304.1 | csasusasusdTsdGsdGs(5MdC)sdAsdTsdGs(5MdC)sdTsdGsascscscsu | 4175 |
| HAO1 | A-133305.1 | asgscscscs(5MdC)s(5MdC)sdAs(5MdC)sdAs(5MdC)sdAsdTsdAsdTsusgsgscsa | 4176 |
| HAO1 | A-133306.1 | csusgscsasdTsdGsdGs(5MdC)s(5MdC)sdGsdTsdAsdGs(5MdC)scscscscsa | 4177 |
| HAO1 | A-133307.1 | gsasgscscsdAsdTsdGs(5MdC)sdGs(5MdC)sdTsdGs(5MdC)sdAsusgsgscsc | 4178 |
| HAO1 | A-133308.1 | gscscsgsus(5MdC)s(5MdC)sdAs(5MdC)sdAsdTsdGsdAsdGs(5MdC)scsasusgsc | 4179 |
| HAO1 | A-133309.1 | asgsusgsgs(5MdC)sdAsdAsdGs(5MdC)sdTs(5MdC)sdGs(5MdC)s(5MdC)sgsuscscsa | 4180 |
| HAO1 | A-133310.1 | ascsasgsgs(5MdC)sdTs(5MdC)sdTs(5MdC)sdAs(5MdC)sdAsdGsdTsgsgscsasa | 4181 |
| HAO1 | A-133311.1 | csasgsgsgsdAs(5MdC)sdTsdGsdAs(5MdC)sdAsdGsdGs(5MdC)suscsuscsa | 4182 |
| HAO1 | A-133312.1 | asusgscscs(5MdC)sdGsdTsdTs(5MdC)s(5MdC)s(5MdC)sdAsdGsdGsgsascsusg | 4183 |
| HAO1 | A-133313.1 | gsasascsus(5MdC)sdAsdAs(5MdC)sdAsdTs(5MdC)sdAsdTsdGscscscsgsu | 4184 |
| HAO1 | A-133314.1 | gsasgsgsusdGsdGs(5MdC)s(5MdC)s(5MdC)sdAsdGsdGsdAsdAscsuscsasa | 4185 |
| HAO1 | A-133315.1 | uscsasasusdTsdGsdAsdGsdGsdAsdGsdGsdTsdGsgscscscsa | 4186 |
| HAO1 | A-133316.1 | cscsgscscsdAs(5MdC)sdTsdTs(5MdC)sdTsdTs(5MdC)sdAsdAsususgsasg | 4187 |
| HAO1 | A-133317.1 | csasgsgsas(5MdC)s(5MdC)sdAsdGs(5MdC)sdTsdTs(5MdC)s(5MdC)sdGscscsascsu | 4188 |
| HAO1 | A-133318.1 | ascsgsasasdGsdTsdGs(5MdC)s(5MdC)sdTs(5MdC)sdAsdGsdGsascscsasg | 4189 |
| HAO1 | A-133319.1 | csasgsususdGs(5MdC)sdAsdGs(5MdC)s(5MdC)sdAsdAs(5MdC)sdGsasasgsusg | 4190 |
| HAO1 | A-133320.1 | usgsusasgsdAsdTsdAsdTsdAs(5MdC)sdAsdGsdTsdTsgscsasgsc | 4191 |
| HAO1 | A-133321.1 | uscsuscsgsdGsdTs(5MdC)s(5MdC)sdTsdTsdGsdTsdAsdGsasusasusa | 4192 |
| HAO1 | A-133322.1 | ususcsususdGsdGsdTsdGsdAs(5MdC)sdTsdTs(5MdC)sdTscsgsgsusc | 4193 |
| HAO1 | A-133323.1 | cscsgscsas(5MdC)sdTsdAsdGs(5MdC)sdTsdTs(5MdC)sdTsdTsgsgsusgsa | 4194 |
| HAO1 | A-133324.1 | csususcsus(5MdC)sdTsdGs(5MdC)s(5MdC)sdTsdGs(5MdC)s(5MdC)sdGscsascsusa | 4195 |
| HAO1 | A-133325.1 | csususgsusdAsdGs(5MdC)s(5MdC)s(5MdC)sdAsdTs(5MdC)sdTsdTscsuscsusg | 4196 |
| HAO1 | A-133326.1 | asasasusasdTsdGsdGs(5MdC)s(5MdC)sdTsdTsdGsdTsdAsgscscscsa | 4197 |
| HAO1 | A-133327.1 | usgsuscscsdAs(5MdC)sdTsdGsdTs(5MdC)sdAs(5MdC)sdAsdAsasusasusg | 4198 |
| HAO1 | A-133328.1 | csasgsgsusdAsdAsdGsdGsdTsdGsdTsdGsdTs(5MdC)scsascsusg | 4199 |
| HAO1 | A-133329.1 | gsascsgsgsdTsdTsdGs(5MdC)s(5MdC)s(5MdC)sdAsdGsdGsdTsasasgsgsu | 4200 |
| HAO1 | A-133330.1 | csascsasus(5MdC)sdAsdTs(5MdC)s(5MdC)sdAsdGsdAs(5MdC)sdGsgsususgsc | 4201 |
| HAO1 | A-133331.1 | asasuscsusdGsdTsdTsdAs(5MdC)sdGs(5MdC)sdAs(5MdC)sdAsuscsasusc | 4202 |
| HAO1 | A-133332.1 | gscsgsgscsdAsdGsdTsdTsdTsdGsdAsdAsdTs(5MdC)susgsususa | 4203 |
| HAO1 | A-133333.1 | csusgsasgsdTsdTsdGsdTsdGsdGs(5MdC)sdGsdGs(5MdC)sasgsususu | 4204 |
| HAO1 | A-133334.1 | asasasasusdTsdTsdTsdTs(5MdC)sdAsdTs(5MdC)s(5MdC)sdTsgsasgsusu | 4205 |
| HAO1 | A-133335.1 | usascsusgsdGsdTsdTsdTs(5MdC)sdAsdAsdAsdAsdTsususususc | 4206 |
| HAO1 | A-133336.1 | asasasusgsdAsdTsdAsdAsdAsdGsdTsdAs(5MdC)sdTsgsgsususu | 4207 |
| HAO1 | A-133337.1 | cscsuscsasdGsdGsdAsdGsdAsdAsdAsdAsdTsdGsasusasasa | 4208 |
| HAO1 | A-133338.1 | uscscsasasdAsdAsdTsdTsdTsdTs(5MdC)s(5MdC)sdTs(5MdC)sasgsgsasg | 4209 |
| HAO1 | A-133339.1 | gsuscscsas(5MdC)sdTsdGsdTs(5MdC)sdGsdTs(5MdC)sdTs(5MdC)scsasasasa | 4210 |
| HAO1 | A-133340.1 | asusasusgs(5MdC)sdAsdGs(5MdC)sdAsdAsdGsdTs(5MdC)s(5MdC)sascsusgsu | 4211 |
| HAO1 | A-133341.1 | usususasgs(5MdC)s(5MdC)sdAs(5MdC)sdAsdTsdAsdTsdGs(5MdC)sasgscsasa | 4212 |
| HAO1 | A-133342.1 | usgsgsgsus(5MdC)sdTsdAsdTsdTsdGs(5MdC)sdTsdTsdTsasgscscsa | 4213 |
| HAO1 | A-133343.1 | asgscsusgsdAsdTsdAsdGsdAsdTsdGsdGsdGsdTscsusasusu | 4214 |
| HAO1 | A-133344.1 | gsasusasus(5MdC)sdTsdTs(5MdC)s(5MdC)s(5MdC)sdAsdGs(5MdC)sdTsgsasusasg | 4215 |
| HAO1 | A-133345.1 | csuscsasgs(5MdC)s(5MdC)sdAsdTsdTsdTsdGsdAsdTsdAsuscsususc | 4216 |
| HAO1 | A-133346.1 | gsasusgsus(5MdC)sdAsdGsdTs(5MdC)sdTsdTs(5MdC)sdTs(5MdC)sasgscscsa | 4217 |
| HAO1 | A-133347.1 | csasasususdGsdGs(5MdC)sdAsdAsdTsdGsdAsdTsdGsuscsasgsu | 4218 |
| HAO1 | A-133348.1 | cscscsususdTsdGs(5MdC)sdAsdAs(5MdC)sdAsdAsdTsdTsgsgscsasa | 4219 |
| HAO1 | A-133349.1 | csuscsuscsdAsdAsdAsdAsdTsdGs(5MdC)s(5MdC)s(5MdC)sdTsususgscsa | 4220 |
| HAO1 | A-133350.1 | gsgscsasus(5MdC)sdAsdTs(5MdC)sdAs(5MdC)s(5MdC)sdTs(5MdC)sdTscsasasasa | 4221 |
| HAO1 | A-133351.1 | asascsasgs(5MdC)s(5MdC)sdTs(5MdC)s(5MdC)s(5MdC)sdTsdGsdGs(5MdC)sasuscsasu | 4222 |
| HAO1 | A-133352.1 | asasgscscsdAsdTsdGsdTsdTsdTsdAsdAs(5MdC)sdAsgscscsusc | 4223 |
| HAO1 | A-133353.1 | asasgsasus(5MdC)s(5MdC)s(5MdC)sdAsdTsdTs(5MdC)sdAsdAsdGscscsasusg | 4224 |
| HAO1 | A-133354.1 | ususcsgsas(5MdC)sdAs(5MdC)s(5MdC)sdAsdAsdGsdAsdTs(5MdC)scscsasusu | 4225 |
| HAO1 | A-133355.1 | uscsgsasgs(5MdC)s(5MdC)s(5MdC)s(5MdC)sdAsdTsdGsdAsdTsdTscsgsascsa | 4226 |
| HAO1 | A-133356.1 | asuscsgsasdGsdTsdTsdGsdTs(5MdC)sdGsdAsdGs(5MdC)scscscsasu | 4227 |
| HAO1 | A-133357.1 | gsgscsusgsdGs(5MdC)sdAs(5MdC)s(5MdC)s(5MdC)s(5MdC)sdAsdTs(5MdC)sgsasgsusu | 4228 |
| HAO1 | A-133358.1 | asascsasus(5MdC)sdAsdAsdTsdAsdGsdTsdGsdGs(5MdC)susgsgscsa | 4229 |
| HAO1 | A-133359.1 | asusususcsdTsdGsdGs(5MdC)sdAsdGsdAsdAs(5MdC)sdAsuscsasasu | 4230 |
| HAO1 | A-133360.1 | asgscscsus(5MdC)s(5MdC)sdAs(5MdC)sdAsdAsdTsdTsdTs(5MdC)susgsgscsa | 4231 |
| HAO1 | A-133361.1 | csususcscs(5MdC)sdTsdTs(5MdC)s(5MdC)sdAs(5MdC)sdAsdGs(5MdC)scsuscscsa | 4232 |
| HAO1 | A-133362.1 | asasgsascsdTsdTs(5MdC)s(5MdC)sdAs(5MdC)s(5MdC)sdTsdTs(5MdC)scscsususc | 4233 |
| HAO1 | A-133363.1 | cscsgsuscs(5MdC)sdAsdGsdGsdAsdAsdGsdAs(5MdC)sdTsuscscsasc | 4234 |
| HAO1 | A-133364.1 | uscscsgscsdAs(5MdC)sdAs(5MdC)s(5MdC)s(5MdC)s(5MdC)s(5MdC)sdGsdTscscsasgsg | 4235 |
| HAO1 | A-133365.1 | csasuscsasdGsdTsdGs(5MdC)s(5MdC)sdTsdTsdTs(5MdC)s(5MdC)sgscsascsa | 4236 |
| HAO1 | A-133366.1 | asgscsususdTs(5MdC)sdAsdGsdAsdAs(5MdC)sdAsdTs(5MdC)sasgsusgsc | 4237 |
| HAO1 | A-133367.1 | csasasgsasdGs(5MdC)s(5MdC)sdAsdGsdAsdGs(5MdC)sdTsdTsuscsasgsa | 4238 |
| HAO1 | A-133368.1 | ascsasgscs(5MdC)sdTsdTsdGsdGs(5MdC)sdGs(5MdC)s(5MdC)sdAsasgsasgsc | 4239 |
| HAO1 | A-133369.1 | uscscscscsdAs(5MdC)sdAsdAsdAs(5MdC)sdAs(5MdC)sdAsdGscscsususg | 4240 |
| HAO1 | A-133370.1 | ascsgsasusdTsdGsdGsdTs(5MdC)sdTs(5MdC)s(5MdC)s(5MdC)s(5MdC)sascsasasa | 4241 |
| HAO1 | A-133371.1 | usasasgscs(5MdC)s(5MdC)s(5MdC)sdAsdAsdAs(5MdC)sdGsdAsdTsusgsgsusc | 4242 |
| HAO1 | A-133372.1 | cscscsusgsdGsdAsdAsdAsdGs(5MdC)sdTsdAsdAsdGscscscscsa | 4243 |
| HAO1 | A-133373.1 | csascscsusdTsdTs(5MdC)sdTs(5MdC)s(5MdC)s(5MdC)s(5MdC)s(5MdC)sdTsgsgsasasa | 4244 |
| HAO1 | A-133374.1 | gsascsasus(5MdC)sdTsdTsdGsdAsdAs(5MdC)sdAs(5MdC)s(5MdC)susususcsu | 4245 |
| HAO1 | A-133375.1 | usasgsusasdTs(5MdC)sdTs(5MdC)sdGsdAsdGsdGsdAs(5MdC)sasuscsusu | 4246 |
| HAO1 | A-133376.1 | gsasasusus(5MdC)sdTsdTs(5MdC)s(5MdC)sdTsdTsdTsdAsdGsusasuscsu | 4247 |
| HAO1 | A-133377.1 | gsgscscsasdAs(5MdC)s(5MdC)sdGsdGsdAsdAsdTsdTs(5MdC)sususcscsu | 4248 |
| HAO1 | A-133378.1 | csuscsasgsdAsdGs(5MdC)s(5MdC)sdAsdTsdGsdGs(5MdC)s(5MdC)sasascscsg | 4249 |
| HAO1 | A-133379.1 | asususcsusdGsdGs(5MdC)sdAs(5MdC)s(5MdC)s(5MdC)sdAs(5MdC)sdTscsasgsasg | 4250 |
| HAO1 | A-133380.1 | asusgsascsdTsdTsdTs(5MdC)sdAs(5MdC)sdAsdTsdTs(5MdC)susgsgscsa | 4251 |
| HAO1 | A-133381.1 | gsuscsususdGsdTs(5MdC)sdGsdAsdTsdGsdAs(5MdC)sdTsususcsasc | 4252 |
| HAO1 | A-133382.1 | usususcscsdTs(5MdC)sdAs(5MdC)s(5MdC)sdAsdAsdTsdGsdTscsususgsu | 4253 |
| HAO1 | A-133383.1 | csasasasgsdGsdAsdTsdTsdTsdTsdTs(5MdC)s(5MdC)sdTscsascscsa | 4254 |
| HAO1 | A-133384.1 | ususgsgsasdAsdAs(5MdC)sdGsdGs(5MdC)s(5MdC)sdAsdAsdAsgsgsasusu | 4255 |
| HAO1 | A-133385.1 | gscsascsusdGsdTs(5MdC)sdAsdGsdAsdTs(5MdC)sdTsdTsgsgsasasa | 4256 |
| HAO1 | A-133386.1 | asasasusasdTsdTsdGsdTsdGs(5MdC)sdAs(5MdC)sdTsdGsuscsasgsa | 4257 |
| HAO1 | A-133387.1 | usascsasgsdAsdTsdGsdGsdGsdAsdAsdAsdAsdTsasususgsu | 4258 |
| HAO1 | A-133388.1 | usgsasasasdAsdAsdAsdAsdAsdTsdAsdAsdTsdAscsasgsasu | 4259 |
| HAO1 | A-133389.1 | usasasusas(5MdC)sdAsdTsdGs(5MdC)sdTsdGsdAsdAsdAsasasasasa | 4260 |
| HAO1 | A-133390.1 | csuscsususdTsdGsdTs(5MdC)sdAsdAsdGsdTsdAsdAsusascsasu | 4261 |
| HAO1 | A-133391.1 | gscsascsasdGsdTsdGsdTs(5MdC)sdTs(5MdC)sdTsdTsdTsgsuscsasa | 4262 |
| HAO1 | A-133392.1 | usgsgsuscsdAs(5MdC)s(5MdC)s(5MdC)sdTs(5MdC)sdTsdGs(5MdC)sdAscsasgsusg | 4263 |
| HAO1 | A-133393.1 | ususascsasdGsdAs(5MdC)sdTsdGsdTsdGsdGsdTs(5MdC)sascscscsu | 4264 |
| HAO1 | A-133394.1 | ususgsasasdGsdTsdGsdGsdGsdGsdAsdAsdTsdTsascsasgsa | 4265 |
| HAO1 | A-133395.1 | cscscsususdTsdGsdTsdAsdTsdTsdGsdAsdAsdGsusgsgsgsg | 4266 |
| HAO1 | A-133396.1 | asasasgsasdAs(5MdC)sdGsdAs(5MdC)sdAs(5MdC)s(5MdC)s(5MdC)sdTsususgsusa | 4267 |
| HAO1 | A-133397.1 | usasusususdTsdGsdTsdTsdGsdGsdAsdAsdAsdAsgsasascsg | 4268 |
| HAO1 | A-133398.1 | asasgsgsgsdAsdTsdTsdGs(5MdC)sdTsdAsdTsdTsdTsusgsususg | 4269 |
| HAO1 | A-133399.1 | gscsasasusdGsdAsdAsdAsdTsdAsdAsdAsdAsdGsgsgsasusu | 4270 |
| HAO1 | A-133400.1 | asasasasgsdTs(5MdC)sdAsdAsdAsdAsdGs(5MdC)sdAsdAsusgsasasa | 4271 |
| HAO1 | A-133401.1 | gsascsascs(5MdC)s(5MdC)sdAsdTsdTsdGsdAsdAsdAsdAsgsuscsasa | 4272 |
| HAO1 | A-133402.1 | asasasgsgsdTsdTs(5MdC)s(5MdC)sdTsdAsdGsdGsdAs(5MdC)sascscscsa | 4273 |
| HAO1 | A-133403.1 | usususcsusdTsdTs(5MdC)sdTsdAsdAsdAsdAsdGsdGsususcscsu | 4274 |
| HAO1 | A-133404.1 | usgsasasasdGsdTs(5MdC)s(5MdC)sdAsdTsdTsdTs(5MdC)sdTsususcsusa | 4275 |
| HAO1 | A-133405.1 | usasusasusdTsdTs(5MdC)s(5MdC)sdAsdGsdGsdAsdTsdGsasasasgsu | 4276 |
| HAO1 | A-133406.1 | usasascsasdGsdTsdTsdAsdAsdTsdAsdTsdAsdTsususcscsa | 4277 |
| HAO1 | A-133407.1 | gsusususus(5MdC)sdTsdTsdTsdTsdTsdAsdAs(5MdC)sdAsgsususasa | 4278 |
| HAO1 | A-133408.1 | csascsasusdTsdTsdTs(5MdC)sdAsdAsdTsdGsdTsdTsususcsusu | 4279 |
| HAO1 | A-133409.1 | ascsgsususdGsdTs(5MdC)sdTsdAsdAsdAs(5MdC)sdAs(5MdC)sasusususu | 4280 |
| HAO1 | A-133410.1 | csasgsgsgsdGsdAsdTsdGsdAs(5MdC)sdGsdTsdTsdGsuscsusasa | 4281 |
| HAO1 | A-133411.1 | csascsususdTsdAsdGs(5MdC)s(5MdC)sdTsdGs(5MdC)s(5MdC)sdAsgsgsgsgsa | 4282 |
| HAO1 | A-133412.1 | asasasgsgsdAsdTsdAs(5MdC)sdAsdGs(5MdC)sdAs(5MdC)sdTsususasgsc | 4283 |
| HAO1 | A-133413.1 | csasasususdTsdTsdAs(5MdC)sdTsdAsdAsdAsdGsdGsasusascsa | 4284 |
| HAO1 | A-133414.1 | ususgscsusdAs(5MdC)s(5MdC)sdTs(5MdC)s(5MdC)sdAsdAsdTsdTsususascsu | 4285 |
| HAO1 | A-133415.1 | csascscsusdTsdAsdGsdTsdGsdTsdTsdTsdGs(5MdC)susascscsu | 4286 |
| HAO1 | A-133416.1 | uscsasususdAsdTs(5MdC)sdTsdTsdTsdTs(5MdC)sdAs(5MdC)scsususasg | 4287 |
| HAO1 | A-133417.1 | asascsasasdTsdGsdAsdGsdAsdTs(5MdC)sdAsdTsdTsasuscsusu | 4288 |
| HAO1 | A-133418.1 | usascsasgsdGsdTsdTsdAsdAsdTsdAsdAsdAs(5MdC)sasasusgsa | 4289 |
| HAO1 | A-133419.1 | gsusasasas(5MdC)sdAsdGsdAsdAsdTsdAs(5MdC)sdAsdGsgsususasa | 4290 |
| HAO1 | A-133420.1 | usususasasdAsdGsdAs(5MdC)sdAsdTsdGsdTsdAsdAsascsasgsa | 4291 |
| HAO1 | A-133421.1 | asasgsasas(5MdC)s(5MdC)sdAs(5MdC)sdTsdGsdTsdTsdTsdTsasasasgsa | 4292 |
| HAO1 | A-133422.1 | csususascsdAsdAsdTsdTsdTsdAsdAsdGsdAsdAscscsascsu | 4293 |
| HAO1 | A-133423.1 | csusususgsdAsdAs(5MdC)s(5MdC)sdTsdGsdAsdGs(5MdC)sdTsusascsasa | 4294 |
| HAO1 | A-133424.1 | asususascs(5MdC)sdAsdAs(5MdC)sdAs(5MdC)sdTsdTsdTsdGsasascscsu | 4295 |
| HAO1 | A-133425.1 | usgsusgsasdAsdTs(5MdC)sdAsdGsdGs(5MdC)sdAsdTsdTsascscsasa | 4296 |
| HAO1 | A-133426.1 | uscsuscsasdAsdAsdGsdTsdTsdGsdTsdGsdAsdAsuscsasgsg | 4297 |
| HAO1 | A-133427.1 | csasgsusgs(5MdC)sdTsdAs(5MdC)s(5MdC)sdTsdTs(5MdC)sdTs(5MdC)sasasasgsu | 4298 |
| HAO1 | A-133428.1 | ususcscsasdAsdTsdTs(5MdC)sdTs(5MdC)sdTs(5MdC)s(5MdC)sdAsgsusgscsu | 4299 |
| HAO1 | A-133429.1 | cscsgscscsdAs(5MdC)s(5MdC)s(5MdC)sdAsdTsdTs(5MdC)s(5MdC)sdAsasususcsu | 4300 |
| HAO1 | A-133430.1 | uscsascscsdAsdAsdTsdTsdAs(5MdC)s(5MdC)sdGs(5MdC)s(5MdC)sascscscsa | 4301 |
| HAO1 | A-133431.1 | asususcsasdAsdAsdGsdAsdAsdGsdTsdAsdTs(5MdC)sascscsasa | 4302 |
| HAO1 | A-133432.1 | usgsgsasasdAsdTs(5MdC)sdTsdAs(5MdC)sdAsdTsdTs(5MdC)sasasasgsa | 4303 |
| HAO1 | A-133433.1 | asasgsasusdGsdTsdGsdAsdTsdTsdGsdGsdAsdAsasuscsusa | 4304 |
| HAO1 | A-133434.1 | ususcsasgsdAs(5MdC)sdAs(5MdC)sdTsdAsdAsdAsdGsdAsusgsusgsa | 4305 |
| HAO1 | A-133435.1 | csasusususdGsdGsdAsdTsdAsdTsdAsdTsdTs(5MdC)sasgsascsa | 4306 |
| HAO1 | A-133436.1 | csasuscscsdTsdAsdAsdAsdAs(5MdC)sdAsdTsdTsdTsgsgsasusa | 4307 |
| HAO1 | A-133437.1 | asasgsusasdAs(5MdC)sdAsdTsdAs(5MdC)sdAsdTs(5MdC)s(5MdC)susasasasa | 4308 |
| HAO1 | A-133438.1 | usususcsus(5MdC)sdTs(5MdC)sdTsdAsdAsdGsdAsdAsdGsusasascsa | 4309 |
| HAO1 | A-133439.1 | asasasusgs(5MdC)sdTsdTsdTsdAsdTsdTsdTs(5MdC)sdTscsuscsusa | 4310 |

TABLE 14
Unmodified antisense polynucleotides targeting HAO1.

| | Oligo | Oligo | SEQ ID | mRNA Target | SEQ ID | |
|---|---|---|---|---|---|---|
| Target | Name | transSeq | NO: | sequence | NO: | Position |
| HAO1 | A-133284.1 | GGGAGCAUUUUCACAGGUUA | 4319 | UAACCUGUGAAAAUGCUCCC | 4475 | 13 |
| HAO1 | A-133285.1 | AAUUAGCCGGGGGAGCAUUU | 4320 | AAAUGCUCCCCCGGCUAAUU | 4476 | 23 |
| HAO1 | A-133286.1 | AUCAUUGAUACAAAUUAGCC | 4321 | GGCUAAUUUGUAUCAAUGAU | 4477 | 35 |
| HAO1 | A-133287.1 | GUUGUUCAUAAUCAUUGAUA | 4322 | UAUCAAUGAUUAUGAACAAC | 4478 | 45 |
| HAO1 | A-133288.1 | GAUUUAGCAUGUUGUUCAUA | 4323 | UAUGAACAACAUGCUAAAUC | 4479 | 55 |
| HAO1 | A-133289.1 | UUGGAAGUACUGAUUUAGCA | 4324 | UGCUAAAUCAGUACUUCCAA | 4480 | 66 |
| HAO1 | A-133290.1 | CAUAUAUAGACUUUGGAAGU | 4325 | ACUUCCAAAGUCUAUAUAUG | 4481 | 78 |
| HAO1 | A-133291.1 | CUGUAAUAGUCAUAUAUAGA | 4326 | UCUAUAUAUGACUAUUACAG | 4482 | 88 |
| HAO1 | A-133292.1 | UUGCCCCAGACCUGUAAUAG | 4327 | CUAUUACAGGUCUGGGGCAA | 4483 | 99 |
| HAO1 | A-133293.1 | UUCUUCAUCAUUUGCCCCAG | 4328 | CUGGGGCAAAUGAUGAAGAA | 4484 | 110 |
| HAO1 | A-133294.1 | UAUCAGCCAAAGUUUCUUCA | 4329 | UGAAGAAACUUUGGCUGAUA | 4485 | 123 |
| HAO1 | A-133295.1 | GCUGCAAUAUUAUCAGCCAA | 4330 | UUGGCUGAUAAUAUUGCAGC | 4486 | 133 |
| HAO1 | A-133296.1 | AUCUGGAAAAUGCUGCAAUA | 4331 | UAUUGCAGCAUUUUCCAGAU | 4487 | 144 |
| HAO1 | A-133297.1 | GAUACAGCUUCCAUCUGGAA | 4332 | UUCCAGAUGGAAGCUGUAUC | 4488 | 156 |
| HAO1 | A-133298.1 | GAGCAUCCUUGGAUACAGCU | 4333 | AGCUGUAUCCAAGGAUGCUC | 4489 | 167 |
| HAO1 | A-133299.1 | CAACAUUCCGGAGCAUCCUU | 4334 | AAGGAUGCUCCGGAAUGUUG | 4490 | 177 |
| HAO1 | A-133300.1 | GAUCUGUUUCAGCAACAUUC | 4335 | GAAUGUUGCUGAAACAGAUC | 4491 | 189 |
| HAO1 | A-133301.1 | AGAAGUCGACAGAUCUGUUU | 4336 | AAACAGAUCUGUCGACUUCU | 4492 | 200 |
| HAO1 | A-133302.1 | UGUCCUAAAACAGAAGUCGA | 4337 | UCGACUUCUGUUUUAGGACA | 4493 | 211 |
| HAO1 | A-133303.1 | UGCUGACCCUCUGUCCUAAA | 4338 | UUUAGGACAGAGGGUCAGCA | 4494 | 222 |
| HAO1 | A-133304.1 | CAUAUUGGCAUGCUGACCCU | 4339 | AGGGUCAGCAUGCCAAUAUG | 4495 | 232 |
| HAO1 | A-133305.1 | AGCCCCCACACAUAUUGGCA | 4340 | UGCCAAUAUGUGUGGGGGCU | 4496 | 242 |
| HAO1 | A-133306.1 | CUGCAUGGCCGUAGCCCCCA | 4341 | UGGGGGCUACGGCCAUGCAG | 4497 | 254 |
| HAO1 | A-133307.1 | GAGCCAUGCGCUGCAUGGCC | 4342 | GGCCAUGCAGCGCAUGGCUC | 4498 | 264 |
| HAO1 | A-133308.1 | GCCGUCCACAUGAGCCAUGC | 4343 | GCAUGGCUCAUGUGGACGGC | 4499 | 275 |
| HAO1 | A-133309.1 | AGUGGCAAGCUCGCCGUCCA | 4344 | UGGACGGCGAGCUUGCCACU | 4500 | 287 |
| HAO1 | A-133310.1 | ACAGGCUCUCACAGUGGCAA | 4345 | UUGCCACUGUGAGAGCCUGU | 4501 | 299 |
| HAO1 | A-133311.1 | CAGGGACUGACAGGCUCUCA | 4346 | UGAGAGCCUGUCAGUCCCUG | 4502 | 308 |
| HAO1 | A-133312.1 | AUGCCCGUUCCCAGGGACUG | 4347 | CAGUCCCUGGGAACGGGCAU | 4503 | 319 |
| HAO1 | A-133313.1 | GAACUCAACAUCAUGCCCGU | 4348 | ACGGGCAUGAUGUUGAGUUC | 4504 | 331 |
| HAO1 | A-133314.1 | GAGGUGGCCCAGGAACUCAA | 4349 | UUGAGUUCCUGGGCCACCUC | 4505 | 343 |
| HAO1 | A-133315.1 | UCAAUUGAGGAGGUGGCCCA | 4350 | UGGGCCACCUCCUCAAUUGA | 4506 | 352 |
| HAO1 | A-133316.1 | CCGCCACUUCUUCAAUUGAG | 4351 | CUCAAUUGAAGAAGUGGCGG | 4507 | 363 |
| HAO1 | A-133317.1 | CAGGACCAGCUUCCGCCACU | 4352 | AGUGGCGGAAGCUGGUCCUG | 4508 | 375 |
| HAO1 | A-133318.1 | ACGAAGUGCCUCAGGACCAG | 4353 | CUGGUCCUGAGGCACUUCGU | 4509 | 386 |
| HAO1 | A-133319.1 | CAGUUGCAGCCAACGAAGUG | 4354 | CACUUCGUUGGCUGCAACUG | 4510 | 398 |
| HAO1 | A-133320.1 | UGUAGAUAUACAGUUGCAGC | 4355 | GCUGCAACUGUAUAUCUACA | 4511 | 408 |
| HAO1 | A-133321.1 | UCUCGGUCCUUGUAGAUAUA | 4356 | UAUAUCUACAAGGACCGAGA | 4512 | 418 |
| HAO1 | A-133322.1 | UUCUUGGUGACUUCUCGGUC | 4357 | GACCGAGAAGUCACCAAGAA | 4513 | 430 |
| HAO1 | A-133323.1 | CCGCACUAGCUUCUUGGUGA | 4358 | UCACCAAGAAGCUAGUGCGG | 4514 | 440 |
| HAO1 | A-133324.1 | CUUCUCUGCCUGCCGCACUA | 4359 | UAGUGCGGCAGGCAGAGAAG | 4515 | 452 |
| HAO1 | A-133325.1 | CUUGUAGCCCAUCUUCUCUG | 4360 | CAGAGAAGAUGGGCUACAAG | 4516 | 464 |
| HAO1 | A-133326.1 | AAAUAUGGCCUUGUAGCCCA | 4361 | UGGGCUACAAGGCCAUAUUU | 4517 | 473 |
| HAO1 | A-133327.1 | UGUCCACUGUCACAAAUAUG | 4362 | CAUAUUUGUGACAGUGGACA | 4518 | 486 |
| HAO1 | A-133328.1 | CAGGUAAGGUGUGUCCACUG | 4363 | CAGUGGACACACCUUACCUG | 4519 | 497 |
| HAO1 | A-133329.1 | GACGGUUGCCCAGGUAAGGU | 4364 | ACCUUACCUGGGCAACCGUC | 4520 | 507 |
| HAO1 | A-133330.1 | CACAUCAUCCAGACGGUUGC | 4365 | GCAACCGUCUGGAUGAUGUG | 4521 | 518 |
| HAO1 | A-133331.1 | AAUCUGUUACGCACAUCAUC | 4366 | GAUGAUGUGCGUAACAGAUU | 4522 | 529 |
| HAO1 | A-133332.1 | GCGGCAGUUUGAAUCUGUUA | 4367 | UAACAGAUUCAAACUGCCGC | 4523 | 540 |
| HAO1 | A-133333.1 | CUGAGUUGUGGCGGCAGUUU | 4368 | AAACUGCCGCCACAACUCAG | 4524 | 550 |
| HAO1 | A-133334.1 | AAAAUUUUUCAUCCUGAGUU | 4369 | AACUCAGGAUGAAAAAUUUU | 4525 | 563 |
| HAO1 | A-133335.1 | UACUGGUUUCAAAAUUUUUC | 4370 | GAAAAAUUUUGAAACCAGUA | 4526 | 573 |
| HAO1 | A-133336.1 | AAAUGAUAAAGUACUGGUUU | 4371 | AAACCAGUACUUUAUCAUUU | 4527 | 584 |
| HAO1 | A-133337.1 | CCUCAGGAGAAAAUGAUAAA | 4372 | UUUAUCAUUUUCUCCUGAGG | 4528 | 594 |
| HAO1 | A-133338.1 | UCCAAAAUUUUCCUCAGGAG | 4373 | CUCCUGAGGAAAAUUUUGGA | 4529 | 605 |
| HAO1 | A-133339.1 | GUCCACUGUCGUCUCCAAAA | 4374 | UUUUGGAGACGACAGUGGAC | 4530 | 618 |
| HAO1 | A-133340.1 | AUAUGCAGCAAGUCCACUGU | 4375 | ACAGUGGACUUGCUGCAUAU | 4531 | 629 |
| HAO1 | A-133341.1 | UUUAGCCACAUAUGCAGCAA | 4376 | UUGCUGCAUAUGUGGCUAAA | 4532 | 638 |
| HAO1 | A-133342.1 | UGGGUCUAUUGCUUUAGCCA | 4377 | UGGCUAAAGCAAUAGACCCA | 4533 | 650 |
| HAO1 | A-133343.1 | AGCUGAUAGAUGGGUCUAUU | 4378 | AAUAGACCCAUCUAUCAGCU | 4534 | 660 |
| HAO1 | A-133344.1 | GAUAUCUUCCCAGCUGAUAG | 4379 | CUAUCAGCUGGGAAGAUAUC | 4535 | 671 |
| HAO1 | A-133345.1 | CUCAGCCAUUUGAUAUCUUC | 4380 | GAAGAUAUCAAAUGGCUGAG | 4536 | 682 |
| HAO1 | A-133346.1 | GAUGUCAGUCUUCUCAGCCA | 4381 | UGGCUGAGAAGACUGACAUC | 4537 | 694 |
| HAO1 | A-133347.1 | CAAUUGGCAAUGAUGUCAGU | 4382 | ACUGACAUCAUUGCCAAUUG | 4538 | 705 |
| HAO1 | A-133348.1 | CCCUUUGCAACAAUUGGCAA | 4383 | UUGCCAAUUGUUGCAAAGGG | 4539 | 715 |
| HAO1 | A-133349.1 | CUCUCAAAAUGCCCUUUGCA | 4384 | UGCAAAGGGCAUUUUGAGAG | 4540 | 726 |
| HAO1 | A-133350.1 | GGCAUCAUCACCUCUCAAAA | 4385 | UUUUGAGAGGUGAUGAUGCC | 4541 | 737 |
| HAO1 | A-133351.1 | AACAGCCUCCCUGGCAUCAU | 4386 | AUGAUGCCAGGGAGGCUGUU | 4542 | 749 |
| HAO1 | A-133352.1 | AAGCCAUGUUUAACAGCCUC | 4387 | GAGGCUGUUAAACAUGGCUU | 4543 | 760 |
| HAO1 | A-133353.1 | AAGAUCCCAUUCAAGCCAUG | 4388 | CAUGGCUUGAAUGGGAUCUU | 4544 | 772 |
| HAO1 | A-133354.1 | UUCGACACCAAGAUCCCAUU | 4389 | AAUGGGAUCUUGGUGUCGAA | 4545 | 781 |
| HAO1 | A-133355.1 | UCGAGCCCCAUGAUUCGACA | 4390 | UGUCGAAUCAUGGGGCUCGA | 4547 | 794 |
| HAO1 | A-133356.1 | AUCGAGUUGUCGAGCCCCAU | 4391 | AUGGGGCUCGACAACUCGAU | 4546 | 803 |
| HAO1 | A-133357.1 | GGCUGGCACCCCAUCGAGUU | 4392 | AACUCGAUGGGGUGCCAGCC | 4548 | 815 |
| HAO1 | A-133358.1 | AACAUCAAUAGUGGCUGGCA | 4393 | UGCCAGCCACUAUUGAUGUU | 4549 | 827 |
| HAO1 | A-133359.1 | AUUUCUGGCAGAACAUCAAU | 4394 | AUUGAUGUUCUGCCAGAAAU | 4550 | 838 |
| HAO1 | A-133360.1 | AGCCUCCACAAUUUCUGGCA | 4395 | UGCCAGAAAUUGUGGAGGCU | 4551 | 848 |
| HAO1 | A-133361.1 | CUUCCCUUCCACAGCCUCCA | 4396 | UGGAGGCUGUGGAAGGGAAG | 4552 | 860 |
| HAO1 | A-133362.1 | AAGACUUCCACCUUCCCUUC | 4397 | GAAGGGAAGGUGGAAGUCUU | 4553 | 871 |
| HAO1 | A-133363.1 | CCGUCCAGGAAGACUUCCAC | 4398 | GUGGAAGUCUUCCUGGACGG | 4554 | 880 |
| HAO1 | A-133364.1 | UCCGCACACCCCCGUCCAGG | 4399 | CCUGGACGGGGGUGUGCGGA | 4555 | 891 |
| HAO1 | A-133365.1 | CAUCAGUGCCUUUCCGCACA | 4400 | UGUGCGGAAAGGCACUGAUG | 4556 | 903 |
| HAO1 | A-133366.1 | AGCUUUCAGAACAUCAGUGC | 4401 | GCACUGAUGUUCUGAAAGCU | 4557 | 914 |
| HAO1 | A-133367.1 | CAAGAGCCAGAGCUUUCAGA | 4402 | UCUGAAAGCUCUGGCUCUUG | 4558 | 924 |
| HAO1 | A-133368.1 | ACAGCCUUGGCGCCAAGAGC | 4403 | GCUCUUGGCGCCAAGGCUGU | 4559 | 937 |
| HAO1 | A-133369.1 | UCCCCACAAACACAGCCUUG | 4404 | CAAGGCUGUGUUUGUGGGGA | 4560 | 948 |
| HAO1 | A-133370.1 | ACGAUUGGUCUCCCCACAAA | 4405 | UUUGUGGGGAGACCAAUCGU | 4561 | 958 |
| HAO1 | A-133371.1 | UAAGCCCCAAACGAUUGGUC | 4406 | GACCAAUCGUUUGGGGCUUA | 4562 | 968 |
| HAO1 | A-133372.1 | CCCUGGAAAGCUAAGCCCCA | 4407 | UGGGGCUUAGCUUUCCAGGG | 4563 | 979 |
| HAO1 | A-133373.1 | CACCUUUCUCCCCCUGGAAA | 4408 | UUUCCAGGGGGAGAAAGGUG | 4564 | 990 |
| HAO1 | A-133374.1 | GACAUCUUGAACACCUUUCU | 4409 | AGAAAGGUGUUCAAGAUGUC | 4565 | 1001 |
| HAO1 | A-133375.1 | UAGUAUCUCGAGGACAUCUU | 4410 | AAGAUGUCCUCGAGAUACUA | 4566 | 1013 |
| HAO1 | A-133376.1 | GAAUUCUUCCUUUAGUAUCU | 4411 | AGAUACUAAAGGAAGAAUUC | 4567 | 1025 |
| HAO1 | A-133377.1 | GGCCAACCGGAAUUCUUCCU | 4412 | AGGAAGAAUUCCGGUUGGCC | 4568 | 1034 |
| HAO1 | A-133378.1 | CUCAGAGCCAUGGCCAACCG | 4413 | CGGUUGGCCAUGGCUCUGAG | 4569 | 1045 |
| HAO1 | A-133379.1 | AUUCUGGCACCCACUCAGAG | 4414 | CUCUGAGUGGGUGCCAGAAU | 4570 | 1058 |
| HAO1 | A-133380.1 | AUGACUUUCACAUUCUGGCA | 4415 | UGCCAGAAUGUGAAAGUCAU | 4571 | 1069 |
| HAO1 | A-133381.1 | GUCUUGUCGAUGACUUUCAC | 4416 | GUGAAAGUCAUCGACAAGAC | 4572 | 1078 |
| HAO1 | A-133382.1 | UUUCCUCACCAAUGUCUUGU | 4417 | ACAAGACAUUGGUGAGGAAA | 4573 | 1091 |
| HAO1 | A-133383.1 | CAAAGGAUUUUUCCUCACCA | 4418 | UGGUGAGGAAAAAUCCUUUG | 4574 | 1100 |
| HAO1 | A-133384.1 | UUGGAAACGGCCAAAGGAUU | 4419 | AAUCCUUUGGCCGUUUCCAA | 4575 | 1111 |
| HAO1 | A-133385.1 | GCACUGUCAGAUCUUGGAAA | 4420 | UUUCCAAGAUCUGACAGUGC | 4576 | 1124 |
| HAO1 | A-133386.1 | AAAUAUUGUGCACUGUCAGA | 4421 | UCUGACAGUGCACAAUAUUU | 4577 | 1133 |
| HAO1 | A-133387.1 | UACAGAUGGGAAAAUAUUGU | 4422 | ACAAUAUUUUCCCAUCUGUA | 4578 | 1144 |
| HAO1 | A-133388.1 | UGAAAAAAAAUAAUACAGAU | 4423 | AUCUGUAUUAUUUUUUUUCA | 4579 | 1157 |
| HAO1 | A-133389.1 | UAAUACAUGCUGAAAAAAAA | 4424 | UUUUUUUUCAGCAUGUAUUA | 4580 | 1167 |
| HAO1 | A-133390.1 | CUCUUUGUCAAGUAAUACAU | 4425 | AUGUAUUACUUGACAAAGAG | 4581 | 1179 |
| HAO1 | A-133391.1 | GCACAGUGUCUCUUUGUCAA | 4426 | UUGACAAAGAGACACUGUGC | 4582 | 1188 |
| HAO1 | A-133392.1 | UGGUCACCCUCUGCACAGUG | 4427 | CACUGUGCAGAGGGUGACCA | 4583 | 1200 |
| HAO1 | A-133393.1 | UUACAGACUGUGGUCACCCU | 4428 | AGGGUGACCACAGUCUGUAA | 4584 | 1210 |
| HAO1 | A-133394.1 | UUGAAGUGGGGAAUUACAGA | 4429 | UCUGUAAUUCCCCACUUCAA | 4585 | 1223 |
| HAO1 | A-133395.1 | CCCUUUGUAUUGAAGUGGGG | 4430 | CCCCACUUCAAUACAAAGGG | 4586 | 1232 |
| HAO1 | A-133396.1 | AAAGAACGACACCCUUUGUA | 4431 | UACAAAGGGUGUCGUUCUUU | 4587 | 1243 |
| HAO1 | A-133397.1 | UAUUUUGUUGGAAAAGAACG | 4432 | CGUUCUUUUCCAACAAAAUA | 4588 | 1255 |
| HAO1 | A-133398.1 | AAGGGAUUGCUAUUUUGUUG | 4433 | CAACAAAAUAGCAAUCCCUU | 4589 | 1265 |
| HAO1 | A-133399.1 | GCAAUGAAAUAAAAGGGAUU | 4434 | AAUCCCUUUUAUUUCAUUGC | 4590 | 1277 |
| HAO1 | A-133400.1 | AAAAGUCAAAAGCAAUGAAA | 4435 | UUUCAUUGCUUUUGACUUUU | 4591 | 1288 |
| HAO1 | A-133401.1 | GACACCCAUUGAAAAGUCAA | 4436 | UUGACUUUUCAAUGGGUGUC | 4592 | 1299 |
| HAO1 | A-133402.1 | AAAGGUUCCUAGGACACCCA | 4437 | UGGGUGUCCUAGGAACCUUU | 4593 | 1311 |
| HAO1 | A-133403.1 | UUUCUUUCUAAAAGGUUCCU | 4438 | AGGAACCUUUUAGAAAGAAA | 4594 | 1321 |
| HAO1 | A-133404.1 | UGAAAGUCCAUUUCUUUCUA | 4439 | UAGAAAGAAAUGGACUUUCA | 4595 | 1331 |
| HAO1 | A-133405.1 | UAUAUUUCCAGGAUGAAAGU | 4440 | ACUUUCAUCCUGGAAAUAUA | 4596 | 1344 |
| HAO1 | A-133406.1 | UAACAGUUAAUAUAUUUCCA | 4441 | UGGAAAUAUAUUAACUGUUA | 4597 | 1354 |
| HAO1 | A-133407.1 | GUUUUCUUUUUAACAGUUAA | 4442 | UUAACUGUUAAAAAGAAAAC | 4598 | 1364 |
| HAO1 | A-133408.1 | CACAUUUUCAAUGUUUUCUU | 4443 | AAGAAAACAUUGAAAAUGUG | 4599 | 1376 |
| HAO1 | A-133409.1 | ACGUUGUCUAAACACAUUUU | 4444 | AAAAUGUGUUUAGACAACGU | 4600 | 1388 |
| HAO1 | A-133410.1 | CAGGGGAUGACGUUGUCUAA | 4445 | UUAGACAACGUCAUCCCCUG | 4601 | 1397 |
| HAO1 | A-133411.1 | CACUUUAGCCUGCCAGGGGA | 4446 | UCCCCUGGCAGGCUAAAGUG | 4602 | 1410 |
| HAO1 | A-133412.1 | AAAGGAUACAGCACUUUAGC | 4447 | GCUAAAGUGCUGUAUCCUUU | 4603 | 1421 |
| HAO1 | A-133413.1 | CAAUUUUACUAAAGGAUACA | 4448 | UGUAUCCUUUAGUAAAAUUG | 4604 | 1431 |
| HAO1 | A-133414.1 | UUGCUACCUCCAAUUUUACU | 4449 | AGUAAAAUUGGAGGUAGCAA | 4605 | 1441 |
| HAO1 | A-133415.1 | CACCUUAGUGUUUGCUACCU | 4450 | AGGUAGCAAACACUAAGGUG | 4606 | 1452 |
| HAO1 | A-133416.1 | UCAUUAUCUUUUCACCUUAG | 4451 | CUAAGGUGAAAAGAUAAUGA | 4607 | 1464 |
| HAO1 | A-133417.1 | AACAAUGAGAUCAUUAUCUU | 4452 | AAGAUAAUGAUCUCAUUGUU | 4608 | 1474 |
| HAO1 | A-133418.1 | UACAGGUUAAUAAACAAUGA | 4453 | UCAUUGUUUAUUAACCUGUA | 4609 | 1486 |
| HAO1 | A-133419.1 | GUAAACAGAAUACAGGUUAA | 4454 | UUAACCUGUAUUCUGUUUAC | 4610 | 1496 |
| HAO1 | A-133420.1 | UUUAAAGACAUGUAAACAGA | 4455 | UCUGUUUACAUGUCUUUAAA | 4611 | 1507 |
| HAO1 | A-133421.1 | AAGAACCACUGUUUUAAAGA | 4456 | UCUUUAAAACAGUGGUUCUU | 4612 | 1519 |
| HAO1 | A-133422.1 | CUUACAAUUUAAGAACCACU | 4457 | AGUGGUUCUUAAAUUGUAAG | 4613 | 1529 |
| HAO1 | A-133423.1 | CUUUGAACCUGAGCUUACAA | 4458 | UUGUAAGCUCAGGUUCAAAG | 4614 | 1542 |
| HAO1 | A-133424.1 | AUUACCAACACUUUGAACCU | 4459 | AGGUUCAAAGUGUUGGUAAU | 4615 | 1552 |
| HAO1 | A-133425.1 | UGUGAAUCAGGCAUUACCAA | 4460 | UUGGUAAUGCCUGAUUCACA | 4616 | 1564 |
| HAO1 | A-133426.1 | UCUCAAAGUUGUGAAUCAGG | 4461 | CCUGAUUCACAACUUUGAGA | 4617 | 1573 |
| HAO1 | A-133427.1 | CAGUGCUACCUUCUCAAAGU | 4462 | ACUUUGAGAAGGUAGCACUG | 4618 | 1584 |
| HAO1 | A-133428.1 | UUCCAAUUCUCUCCAGUGCU | 4463 | AGCACUGGAGAGAAUUGGAA | 4619 | 1597 |
| HAO1 | A-133429.1 | CCGCCACCCAUUCCAAUUCU | 4464 | AGAAUUGGAAUGGGUGGCGG | 4620 | 1607 |
| HAO1 | A-133430.1 | UCACCAAUUACCGCCACCCA | 4465 | UGGGUGGCGGUAAUUGGUGA | 4621 | 1617 |
| HAO1 | A-133431.1 | AUUCAAAGAAGUAUCACCAA | 4466 | UUGGUGAUACUUCUUUGAAU | 4622 | 1630 |
| HAO1 | A-133432.1 | UGGAAAUCUACAUUCAAAGA | 4467 | UCUUUGAAUGUAGAUUUCCA | 4623 | 1641 |
| HAO1 | A-133433.1 | AAGAUGUGAUUGGAAAUCUA | 4468 | UAGAUUUCCAAUCACAUCUU | 4624 | 1651 |
| HAO1 | A-133434.1 | UUCAGACACUAAAGAUGUGA | 4469 | UCACAUCUUUAGUGUCUGAA | 4625 | 1662 |
| HAO1 | A-133435.1 | CAUUUGGAUAUAUUCAGACA | 4470 | UGUCUGAAUAUAUCCAAAUG | 4626 | 1674 |
| HAO1 | A-133436.1 | CAUCCUAAAACAUUUGGAUA | 4471 | UAUCCAAAUGUUUUAGGAUG | 4627 | 1684 |
| HAO1 | A-133437.1 | AAGUAACAUACAUCCUAAAA | 4472 | UUUUAGGAUGUAUGUUACUU | 4628 | 1694 |
| HAO1 | A-133438.1 | UUUCUCUCUAAGAAGUAACA | 4473 | UGUUACUUCUUAGAGAGAAA | 4629 | 1706 |
| HAO1 | A-133439.1 | AAAUGCUUUAUUUCUCUCUA | 4474 | UAGAGAGAAAUAAAGCAUUU | 4630 | 1716 |

TABLE 15
Unmodified Sense and Antisense Strand Sequences of PRODH2 dsRNA Agents

| Duplex | Sense Sequence | SEQ ID | Range in | Antisense Sequence | SEQ ID | Range in |
|---|---|---|---|---|---|---|
| Name | 5′ to 3′ | NO: | NM_021232.2 | 5′ to 3′ | NO: | NM_021232.2 |
| AD-1553630 | AGAACCUUCCCUGGUGUGGAU | 4651 | 3-23 | AUCCACACCAGGGAAGGUUCUCU | 4786 | 1-23 |
| AD-1553635 | CUUCCCUGGUGUGGAGGCAGU | 4652 | 8-28 | ACUGCCUCCACACCAGGGAAGGU | 4787 | 86-28 |
| AD-1553662 | CAGGAUGCUCCGGACCUGUUU | 4653 | 37-57 | AAACAGGUCCGGAGCAUCCUGGG | 4788 | 35-57 |
| AD-1553667 | UGCUCCGGACCUGUUACGUGU | 4654 | 42-62 | ACACGUAACAGGUCCGGAGCAUC | 4789 | 40-62 |
| AD-1553674 | GACCUGUUACGUGCUCUGUUU | 4655 | 49-69 | AAACAGAGCACGUAACAGGUCCG | 4790 | 47-69 |
| AD-1553681 | UACGUGCUCUGUUCCCAAGCU | 4656 | 56-76 | AGCUUGGGAACAGAGCACGUAAC | 4791 | 54-76 |
| AD-1553686 | GCUCUGUUCCCAAGCUGGUCU | 4657 | 61-81 | AGACCAGCUUGGGAACAGAGCAC | 4792 | 59-81 |
| AD-1553691 | CUGGCAGUCCCUGAGCUUUGU | 4658 | 94-114 | ACAAAGCUCAGGGACUGCCAGCC | 4793 | 92-114 |
| AD-1553697 | GUCCCUGAGCUUUGAUGGCGU | 4659 | 100-120 | ACGCCAUCAAAGCUCAGGGACUG | 4794 | 98-120 |
| AD-1553701 | GCCUUCCACCUUAAGGGCACU | 4660 | 122-142 | AGUGCCCUUAAGGUGGAAGGCCC | 4795 | 120-142 |
| AD-1553707 | CACCUUAAGGGCACAGGAGAU | 4661 | 128-148 | AUCUCCUGUGCCCUUAAGGUGGA | 4796 | 126-148 |
| AD-1553715 | GGGCACAGGAGAGCUGACACU | 4662 | 136-156 | AGUGUCAGCUCUCCUGUGCCCUU | 4797 | 134-156 |
| AD-1553722 | GGAGAGCUGACACGGGCCUUU | 4663 | 143-163 | AAAGGCCCGUGUCAGCUCUCCUG | 4798 | 141-163 |
| AD-1553730 | GACACGGGCCUUGCUGGUUCU | 4664 | 151-171 | AGAACCAGCAAGGCCCGUGUCAG | 4799 | 149-171 |
| AD-1553739 | CUUGCUGGUUCUCCGGCUGUU | 4665 | 160-180 | AACAGCCGGAGAACCAGCAAGGC | 4800 | 158-180 |
| AD-1553745 | GGUUCUCCGGCUGUGUGCCUU | 4666 | 166-186 | AAGGCACACAGCCGGAGAACCAG | 4801 | 164-186 |
| AD-1553751 | CUCGUCACUCACGGGCUGUUU | 4667 | 194-214 | AAACAGCCCGUGAGUGACGAGUG | 4802 | 192-214 |
| AD-1553758 | CUCACGGGCUGUUGCUCCAGU | 4668 | 201-221 | ACUGGAGCAACAGCCCGUGAGUG | 4803 | 199-221 |
| AD-1553769 | UUGCUCCAGGCCUGGUCUCGU | 4669 | 212-232 | ACGAGACCAGGCCUGGAGCAACA | 4804 | 210-232 |
| AD-1553794 | GGCUCUCAGGCGCAUUUCUCU | 4670 | 249-269 | AGAGAAAUGCGCCUGAGAGCCGG | 4805 | 247-269 |
| AD-1553799 | UCAGGCGCAUUUCUCCGAGCU | 4671 | 254-274 | AGCUCGGAGAAAUGCGCCUGAGA | 4806 | 252-274 |
| AD-1553804 | CGCAUUUCUCCGAGCAUCCGU | 4672 | 259-279 | ACGGAUGCUCGGAGAAAUGCGCC | 4807 | 257-279 |
| AD-1553809 | UUCUCCGAGCAUCCGUCUAUU | 4673 | 264-284 | AAUAGACGGAUGCUCGGAGAAAU | 4808 | 262-284 |
| AD-1553815 | GAGCAUCCGUCUAUGGGCAGU | 4674 | 270-290 | ACUGCCCAUAGACGGAUGCUCGG | 4809 | 268-290 |
| AD-1553820 | UCCGUCUAUGGGCAGUUUGUU | 4675 | 275-295 | AACAAACUGCCCAUAGACGGAUG | 4810 | 273-295 |
| AD-1553826 | UAUGGGCAGUUUGUGGCUGGU | 4676 | 281-301 | ACCAGCCACAAACUGCCCAUAGA | 4811 | 279-301 |
| AD-1553832 | CAGUUUGUGGCUGGUGAGACU | 4677 | 287-307 | AGUCUCACCAGCCACAAACUGCC | 4812 | 285-307 |
| AD-1553839 | UGGCUGGUGAGACAGCAGAGU | 4678 | 294-314 | ACUCUGCUGUCUCACCAGCCACA | 4813 | 292-314 |
| AD-1553845 | GUGAGACAGCAGAGGAGGUGU | 4679 | 300-320 | ACACCUCCUCUGCUGUCUCACCA | 4814 | 298-320 |
| AD-1553852 | AGCAGAGGAGGUGAAGGGCUU | 4680 | 307-327 | AAGCCCUUCACCUCCUCUGCUGU | 4815 | 305-327 |
| AD-1553859 | GAGGUGAAGGGCUGCGUGCAU | 4681 | 314-334 | AUGCACGCAGCCCUUCACCUCCU | 4816 | 312-334 |
| AD-1553865 | AAGGGCUGCGUGCAGCAGCUU | 4682 | 320-340 | AAGCUGCUGCACGCAGCCCUUCA | 4817 | 318-340 |
| AD-1553907 | UGCUGGCAGUGCCCACUGAGU | 4683 | 363-383 | ACUCAGUGGGCACUGCCAGCAGU | 4818 | 361-383 |
| AD-1553912 | GCAGUGCCCACUGAGGAGGAU | 4684 | 368-388 | AUCCUCCUCAGUGGGCACUGCCA | 4819 | 366-388 |
| AD-1553922 | CUGAGGAGGAGCCGGACUCUU | 4685 | 378-398 | AAGAGUCCGGCUCCUCCUCAGUG | 4820 | 376-398 |
| AD-1553930 | GAGCCGGACUCUGCUGCCAAU | 4686 | 386-406 | AUUGGCAGCAGAGUCCGGCUCCU | 4821 | 384-406 |
| AD-1553937 | ACUCUGCUGCCAAGAGUGGUU | 4687 | 393-413 | AACCACUCUUGGCAGCAGAGUCC | 4822 | 391-413 |
| AD-1553942 | GCUGCCAAGAGUGGUGAGGCU | 4688 | 398-418 | AGCCUCACCACUCUUGGCAGCAG | 4823 | 396-418 |
| AD-1553951 | AGUGGUGAGGCGUGGUAUGAU | 4689 | 407-427 | AUCAUACCACGCCUCACCACUCU | 4824 | 405-427 |
| AD-1553957 | AACCUCGGUGCUAUGCUGCGU | 4690 | 431-451 | ACGCAGCAUAGCACCGAGGUUCC | 4825 | 429-451 |
| AD-1553962 | CGGUGCUAUGCUGCGGUGUGU | 4691 | 436-456 | ACACACCGCAGCAUAGCACCGAG | 4826 | 434-456 |
| AD-1553967 | CUAUGCUGCGGUGUGUGGACU | 4692 | 441-461 | AGUCCACACACCGCAGCAUAGCA | 4827 | 439-461 |
| AD-1553975 | CGGUGUGUGGACCUGUCACGU | 4693 | 449-469 | ACGUGACAGGUCCACACACCGCA | 4828 | 447-469 |
| AD-1553988 | GGCCAGCCUCAUGCAGCUGAU | 4694 | 499-519 | AUCAGCUGCAUGAGGCUGGCCUC | 4829 | 497-519 |
| AD-1553993 | GCCUCAUGCAGCUGAAGGUGU | 4695 | 504-524 | ACACCUUCAGCUGCAUGAGGCUG | 4830 | 502-524 |
| AD-1553998 | AUGCAGCUGAAGGUGACGGCU | 4696 | 509-529 | AGCCGUCACCUUCAGCUGCAUGA | 4831 | 507-529 |
| AD-1554003 | GCUGAAGGUGACGGCGCUGAU | 4697 | 514-534 | AUCAGCGCCGUCACCUUCAGCUG | 4832 | 512-534 |
| AD-1554009 | GGUGACGGCGCUGACCAGUAU | 4698 | 520-540 | AUACUGGUCAGCGCCGUCACCUU | 4833 | 518-540 |
| AD-1554018 | GCUGACCAGUACUCGGCUCUU | 4699 | 529-549 | AAGAGCCGAGUACUGGUCAGCGC | 4834 | 527-549 |
| AD-1554025 | AGUACUCGGCUCUGUAAGGAU | 4700 | 536-556 | AUCCUUACAGAGCCGAGUACUGG | 4835 | 534-556 |
| AD-1554030 | UCGGCUCUGUAAGGAGCUAGU | 4701 | 541-561 | ACUAGCUCCUUACAGAGCCGAGU | 4836 | 539-561 |
| AD-1554036 | CUGUAAGGAGCUAGCCUCGUU | 4702 | 547-567 | AACGAGGCUAGCUCCUUACAGAG | 4837 | 545-567 |
| AD-1554041 | AGGAGCUAGCCUCGUGGGUCU | 4703 | 552-572 | AGACCCACGAGGCUAGCUCCUUA | 4838 | 550-572 |
| AD-1554048 | AGCCUCGUGGGUCAGAAGGCU | 4704 | 559-579 | AGCCUUCUGACCCACGAGGCUAG | 4839 | 557-579 |
| AD-1554054 | GUGGGUCAGAAGGCCAGGAGU | 4705 | 565-585 | ACUCCUGGCCUUCUGACCCACGA | 4840 | 563-585 |
| AD-1554065 | GGCCAGGAGCCUCCUUGGAGU | 4706 | 576-596 | ACUCCAAGGAGGCUCCUGGCCUU | 4841 | 574-596 |
| AD-1554070 | GGAGCCUCCUUGGAGCUGAGU | 4707 | 581-601 | ACUCAGCUCCAAGGAGGCUCCUG | 4842 | 579-601 |
| AD-1554074 | GAGAGGCUGGCUGAAGCUAUU | 4708 | 605-625 | AAUAGCUUCAGCCAGCCUCUCGG | 4843 | 603-625 |
| AD-1554082 | GGCUGAAGCUAUGGACUCUGU | 4709 | 613-633 | ACAGAGUCCAUAGCUUCAGCCAG | 4844 | 611-633 |
| AD-1554089 | GCUAUGGACUCUGGGCAGAAU | 4710 | 620-640 | AUUCUGCCCAGAGUCCAUAGCUU | 4845 | 618-640 |
| AD-1554097 | CUCUGGGCAGAACCUCCAGGU | 4711 | 628-648 | ACCUGGAGGUUCUGCCCAGAGUC | 4846 | 626-648 |
| AD-1554106 | GAACCUCCAGGUCUCCUGCCU | 4712 | 637-657 | AGGCAGGAGACCUGGAGGUUCUG | 4847 | 635-657 |
| AD-1554111 | UCCAGGUCUCCUGCCUCAAUU | 4713 | 642-662 | AAUUGAGGCAGGAGACCUGGAGG | 4848 | 640-662 |
| AD-1554119 | UCCUGCCUCAAUGCUGAGCAU | 4714 | 650-670 | AUGCUCAGCAUUGAGGCAGGAGA | 4849 | 648-670 |
| AD-1554126 | UCAAUGCUGAGCAGAACCAGU | 4715 | 657-677 | ACUGGUUCUGCUCAGCAUUGAGG | 4850 | 655-677 |
| AD-1554131 | GCUGAGCAGAACCAGCACCUU | 4716 | 662-682 | AAGGUGCUGGUUCUGCUCAGCAU | 4851 | 660-682 |
| AD-1554156 | GCAUCGGGUGGCACAGUAUGU | 4717 | 703-723 | ACAUACUGUGCCACCCGAUGCAG | 4852 | 701-723 |
| AD-1554186 | CUCCUGGUGGAUGCGGAGUAU | 4718 | 743-763 | AUACUCCGCAUCCACCAGGAGCC | 4853 | 741-763 |
| AD-1554193 | UGGAUGCGGAGUACACCUCAU | 4719 | 750-770 | AUGAGGUGUACUCCGCAUCCACC | 4854 | 748-770 |
| AD-1554198 | GCGGAGUACACCUCACUGAAU | 4720 | 755-775 | AUUCAGUGAGGUGUACUCCGCAU | 4855 | 753-775 |
| AD-1554203 | GUACACCUCACUGAACCCUGU | 4721 | 760-780 | ACAGGGUUCAGUGAGGUGUACUC | 4856 | 758-780 |
| AD-1554210 | UCACUGAACCCUGCGCUCUCU | 4722 | 767-787 | AGAGAGCGCAGGGUUCAGUGAGG | 4857 | 765-787 |
| AD-1554216 | AACCCUGCGCUCUCGCUGCUU | 4723 | 773-793 | AAGCAGCGAGAGCGCAGGGUUCA | 4858 | 771-793 |
| AD-1554263 | CCCUGGGUGUGGAACACCUAU | 4724 | 839-859 | AUAGGUGUUCCACACCCAGGGCC | 4859 | 837-859 |
| AD-1554268 | GGUGUGGAACACCUACCAGGU | 4725 | 844-864 | ACCUGGUAGGUGUUCCACACCCA | 4860 | 842-864 |
| AD-1554278 | ACCUACCAGGCCUGUCUAAAU | 4726 | 854-874 | AUUUAGACAGGCCUGGUAGGUGU | 4861 | 852-874 |
| AD-1554287 | GCCUGUCUAAAGGACACAUUU | 4727 | 863-883 | AAAUGUGUCCUUUAGACAGGCCU | 4862 | 861-883 |
| AD-1554292 | UCUAAAGGACACAUUCGAGCU | 4728 | 868-888 | AGCUCGAAUGUGUCCUUUAGACA | 4863 | 866-888 |
| AD-1554298 | GGACACAUUCGAGCGGCUGGU | 4729 | 874-894 | ACCAGCCGCUCGAAUGUGUCCUU | 4864 | 872-894 |
| AD-1554317 | GGCCUUCGGAGUGAAGCUGGU | 4730 | 928-948 | ACCAGCUUCACUCCGAAGGCCAG | 4865 | 926-948 |
| AD-1554323 | CGGAGUGAAGCUGGUACGAGU | 4731 | 934-954 | ACUCGUACCAGCUUCACUCCGAA | 4866 | 932-954 |
| AD-1554328 | UGAAGCUGGUACGAGGUGCAU | 4732 | 939-959 | AUGCACCUCGUACCAGCUUCACU | 4867 | 937-959 |
| AD-1554333 | CUGGUACGAGGUGCAUAUCUU | 4733 | 944-964 | AAGAUAUGCACCUCGUACCAGCU | 4868 | 942-964 |
| AD-1554340 | GAGGUGCAUAUCUGGACAAGU | 4734 | 951-971 | ACUUGUCCAGAUAUGCACCUCGU | 4869 | 949-971 |
| AD-1554346 | CAUAUCUGGACAAGGAGAGAU | 4735 | 957-977 | AUCUCUCCUUGUCCAGAUAUGCA | 4870 | 955-977 |
| AD-1554351 | CUGGACAAGGAGAGAGCGGUU | 4736 | 962-982 | AACCGCUCUCUCCUUGUCCAGAU | 4871 | 960-982 |
| AD-1554371 | GCCCAGCUCCAUGGGAUGGAU | 4737 | 983-1003 | AUCCAUCCCAUGGAGCUGGGCCA | 4872 | 981-1003 |
| AD-1554376 | GCUCCAUGGGAUGGAAGACCU | 4738 | 988-1008 | AGGUCUUCCAUCCCAUGGAGCUG | 4873 | 986-1008 |
| AD-1554379 | CUCAGCCUGACUAUGAGGCCU | 4739 | 1011-1031 | AGGCCUCAUAGUCAGGCUGAGUG | 4874 | 1009-1031 |
| AD-1554387 | GACUAUGAGGCCACCAGUCAU | 4740 | 1019-1039 | AUGACUGGUGGCCUCAUAGUCAG | 4875 | 1017-1039 |
| AD-1554393 | GAGGCCACCAGUCAGAGUUAU | 4741 | 1025-1045 | AUAACUCUGACUGGUGGCCUCAU | 4876 | 1023-1045 |
| AD-1554399 | ACCAGUCAGAGUUACAGCCGU | 4742 | 1031-1051 | ACGGCUGUAACUCUGACUGGUGG | 4877 | 1029-1051 |
| AD-1554404 | UCAGAGUUACAGCCGCUGCCU | 4743 | 1036-1056 | AGGCAGCGGCUGUAACUCUGACU | 4878 | 1034-1056 |
| AD-1554413 | CAGCCGCUGCCUGGAACUGAU | 4744 | 1045-1065 | AUCAGUUCCAGGCAGCGGCUGUA | 4879 | 1043-1065 |
| AD-1554418 | GCUGCCUGGAACUGAUGCUGU | 4745 | 1050-1070 | ACAGCAUCAGUUCCAGGCAGCGG | 4880 | 1048-1070 |
| AD-1554425 | GGAACUGAUGCUGACGCACGU | 4746 | 1057-1077 | ACGUGCGUCAGCAUCAGUUCCAG | 4881 | 1055-1077 |
| AD-1554430 | UGAUGCUGACGCACGUGGCCU | 4747 | 1062-1082 | AGGCCACGUGCGUCAGCAUCAGU | 4882 | 1060-1082 |
| AD-1554433 | CAUGUGCCACCUCAUGGUGGU | 4748 | 1093-1113 | ACCACCAUGAGGUGGCACAUGGG | 4883 | 1091-1113 |
| AD-1554443 | CUCAUGGUGGCUUCCCACAAU | 4749 | 1103-1123 | AUUGUGGGAAGCCACCAUGAGGU | 4884 | 1101-1123 |
| AD-1554451 | GGCUUCCCACAAUGAGGAAUU | 4750 | 1111-1131 | AAUUCCUCAUUGUGGGAAGCCAC | 4885 | 1109-1131 |
| AD-1554456 | CCCACAAUGAGGAAUCUGUUU | 4751 | 1116-1136 | AAACAGAUUCCUCAUUGUGGGAA | 4886 | 1114-1136 |
| AD-1554461 | AAUGAGGAAUCUGUUCGCCAU | 4752 | 1121-1141 | AUGGCGAACAGAUUCCUCAUUGU | 4887 | 1119-1141 |
| AD-1554470 | UCUGUUCGCCAGGCAACCAAU | 4753 | 1130-1150 | AUUGGUUGCCUGGCGAACAGAUU | 4888 | 1128-1150 |
| AD-1554475 | UCGCCAGGCAACCAAGCGCAU | 4754 | 1135-1155 | AUGCGCUUGGUUGCCUGGCGAAC | 4889 | 1133-1155 |
| AD-1554482 | GCAACCAAGCGCAUGUGGGAU | 4755 | 1142-1162 | AUCCCACAUGCGCUUGGUUGCCU | 4890 | 1140-1162 |
| AD-1554487 | CAAGCGCAUGUGGGAGCUGGU | 4756 | 1147-1167 | ACCAGCUCCCACAUGCGCUUGGU | 4891 | 1145-1167 |
| AD-1554492 | GCAUGUGGGAGCUGGGCAUUU | 4757 | 1152-1172 | AAAUGCCCAGCUCCCACAUGCGC | 4892 | 1150-1172 |
| AD-1554497 | UGGGAGCUGGGCAUUCCUCUU | 4758 | 1157-1177 | AAGAGGAAUGCCCAGCUCCCACA | 4893 | 1155-1177 |
| AD-1554505 | GGGCAUUCCUCUGGAUGGGAU | 4759 | 1165-1185 | AUCCCAUCCAGAGGAAUGCCCAG | 4894 | 1163-1185 |
| AD-1554510 | UUCCUCUGGAUGGGACUGUCU | 4760 | 1170-1190 | AGACAGUCCCAUCCAGAGGAAUG | 4895 | 1168-1190 |
| AD-1554515 | CUGGAUGGGACUGUCUGUUUU | 4761 | 1175-1195 | AAAACAGACAGUCCCAUCCAGAG | 4896 | 1173-1195 |
| AD-1554520 | UGGGACUGUCUGUUUCGGACU | 4762 | 1180-1200 | AGUCCGAAACAGACAGUCCCAUC | 4897 | 1178-1200 |
| AD-1554525 | CUGUCUGUUUCGGACAACUUU | 4763 | 1185-1205 | AAAGUUGUCCGAAACAGACAGUC | 4898 | 1183-1205 |
| AD-1554533 | UUCGGACAACUUCUGGGCAUU | 4764 | 1193-1213 | AAUGCCCAGAAGUUGUCCGAAAC | 4899 | 1191-1213 |
| AD-1554542 | CUUCUGGGCAUGUGUGACCAU | 4765 | 1202-1222 | AUGGUCACACAUGCCCAGAAGUU | 4900 | 1200-1222 |
| AD-1554551 | AUGUGUGACCACGUCUCUCUU | 4766 | 1211-1231 | AAGAGAGACGUGGUCACACAUGC | 4901 | 1209-1231 |
| AD-1554557 | GACCACGUCUCUCUAGCACUU | 4767 | 1217-1237 | AAGUGCUAGAGAGACGUGGUCAC | 4902 | 1215-1237 |
| AD-1554562 | GCAGGCCGGCUAUGUAGUGUU | 4768 | 1240-1260 | AACACUACAUAGCCGGCCUGCCC | 4903 | 1238-1260 |
| AD-1554567 | CCGGCUAUGUAGUGUAUAAGU | 4769 | 1245-1265 | ACUUAUACACUACAUAGCCGGCC | 4904 | 1243-1265 |
| AD-1554573 | AUGUAGUGUAUAAGUCCAUUU | 4770 | 1251-1271 | AAAUGGACUUAUACACUACAUAG | 4905 | 1249-1271 |
| AD-1554578 | GUGUAUAAGUCCAUUCCCUAU | 4771 | 1256-1276 | AUAGGGAAUGGACUUAUACACUA | 4906 | 1254-1276 |
| AD-1554584 | AAGUCCAUUCCCUAUGGCUCU | 4772 | 1262-1282 | AGAGCCAUAGGGAAUGGACUUAU | 4907 | 1260-1282 |
| AD-1554591 | UUCCCUAUGGCUCCUUGGAGU | 4773 | 1269-1289 | ACUCCAAGGAGCCAUAGGGAAUG | 4908 | 1267-1289 |
| AD-1554599 | GGCUCCUUGGAGGAGGUAAUU | 4774 | 1277-1297 | AAUUACCUCCUCCAAGGAGCCAU | 4909 | 1275-1297 |
| AD-1554609 | AUCCGGAGGGCCCAGGAGAAU | 4775 | 1307-1327 | AUUCUCCUGGGCCCUCCGGAUCA | 4910 | 1305-1327 |
| AD-1554626 | GAACCGGAGCGUGCUUCAGGU | 4776 | 1324-1344 | ACCUGAAGCACGCUCCGGUUCUC | 4911 | 1322-1344 |
| AD-1554642 | CAGGGUGCCCGCAGGGAACAU | 4777 | 1340-1360 | AUGUUCCCUGCGGGCACCCUGAA | 4912 | 1338-1360 |
| AD-1554653 | CAGGGAACAGGAGCUGCUCAU | 4778 | 1351-1371 | AUGAGCAGCUCCUGUUCCCUGCG | 4913 | 1349-1371 |
| AD-1554658 | AACAGGAGCUGCUCAGCCAAU | 4779 | 1356-1376 | AUUGGCUGAGCAGCUCCUGUUCC | 4914 | 1354-1376 |
| AD-1554663 | GAGCUGCUCAGCCAAGAACUU | 4780 | 1361-1381 | AAGUUCUUGGCUGAGCAGCUCCU | 4915 | 1359-1381 |
| AD-1554668 | GCUCAGCCAAGAACUGUGGCU | 4781 | 1366-1386 | AGCCACAGUUCUUGGCUGAGCAG | 4916 | 1364-1386 |
| AD-1554690 | UGCCAGGAUGCCGAAGGAUAU | 4782 | 1395-1415 | AUAUCCUUCGGCAUCCUGGCAGC | 4917 | 1393-1415 |
| AD-1554696 | UCAUGUGGUCAAUAAAAGUCU | 4783 | 1438-1458 | AGACUUUUAUUGACCACAUGACC | 4918 | 1436-1458 |
| AD-1554704 | UCAAUAAAAGUCCUUAGGUGU | 4784 | 1446-1466 | ACACCUAAGGACUUUUAUUGACC | 4919 | 1444-1466 |
| AD-1554709 | AAAAGUCCUUAGGUGCUGCCU | 4785 | 1451-1471 | AGGCAGCACCUAAGGACUUUUAU | 4920 | 1449-1471 |

TABLE 16
Modified Sense and Antisense Strand Sequences of PRODH2 dsRNA Agents

| | | SEQ | | SEQ | | SEQ |
|---|---|---|---|---|---|---|
| Duplex | | ID | | ID | | ID |
| Name | Sense Sequence 5′ to 3′ | NO: | Anti sense Sequence 5′ to 3′ | NO: | mRNA target sequence | NO: |
| AD-1553630 | asgsaaccUfuCfCfCfugguguggauL96 | 4921 | asUfsccaCfaCfCfagggAfaGfguucuscsu | 5056 | AGAGAACCUUCCCUGGUGUGGAG | 5191 |
| AD-1553635 | csusucccUfgGfUfGfuggaggcaguL96 | 4922 | asCfsugcCfuCfCfacacCfaGfggaagsgsu | 5057 | ACCUUCCCUGGUGUGGAGGCAGC | 5192 |
| AD-1553662 | csasggauGfcUfCfCfggaccuguuuL96 | 4923 | asAfsacaGfgUfCfcggaGfcAfuccugsgsg | 5058 | CCCAGGAUGCUCCGGACCUGUUA | 5193 |
| AD-1553667 | usgscuccGfgAfCfCfuguuacguguL96 | 4924 | asCfsacgUfaAfCfagguCfcGfgagcasusc | 5059 | GAUGCUCCGGACCUGUUACGUGC | 5194 |
| AD-1553674 | gsasccugUfuAfCfGfugcucuguuuL96 | 4925 | asAfsacaGfaGfCfacguAfaCfaggucscsg | 5060 | CGGACCUGUUACGUGCUCUGUUC | 5195 |
| AD-1553681 | usascgugCfuCfUfGfuucccaagcuL96 | 4926 | asGfscuuGfgGfAfacagAfgCfacguasasc | 5061 | GUUACGUGCUCUGUUCCCAAGCU | 5196 |
| AD-1553686 | gscsucugUfuCfCfCfaagcuggucuL96 | 4927 | asGfsaccAfgCfUfugggAfaCfagagcsasc | 5062 | GUGCUCUGUUCCCAAGCUGGUCC | 5197 |
| AD-1553691 | csusggcaGfuCfCfCfugagcuuuguL96 | 4928 | asCfsaaaGfcUfCfagggAfcUfgccagscsc | 5063 | GGCUGGCAGUCCCUGAGCUUUGA | 5198 |
| AD-1553697 | gsuscccuGfaGfCfUfuugauggcguL96 | 4929 | asCfsgccAfuCfAfaagcUfcAfgggacsusg | 5064 | CAGUCCCUGAGCUUUGAUGGCGG | 5199 |
| AD-1553701 | gscscuucCfaCfCfUfuaagggcacuL96 | 4930 | asGfsugcCfcUfUfaaggUfgGfaaggcscsc | 5065 | GGGCCUUCCACCUUAAGGGCACA | 5200 |
| AD-1553707 | csasccuuAfaGfGfGfcacaggagauL96 | 4931 | asUfscucCfuGfUfgcccUfuAfaggugsgsa | 5066 | UCCACCUUAAGGGCACAGGAGAG | 5201 |
| AD-1553715 | gsgsgcacAfgGfAfGfagcugacacuL96 | 4932 | asGfsuguCfaGfCfucucCfuGfugcccsusu | 5067 | AAGGGCACAGGAGAGCUGACACG | 5202 |
| AD-1553722 | gsgsagagCfuGfAfCfacgggccuuuL96 | 4933 | asAfsaggCfcCfGfugucAfgCfucuccsusg | 5068 | CAGGAGAGCUGACACGGGCCUUG | 5203 |
| AD-1553730 | gsascacgGfgCfCfUfugcugguucuL96 | 4934 | asGfsaacCfaGfCfaaggCfcCfgugucsasg | 5069 | CUGACACGGGCCUUGCUGGUUCU | 5204 |
| AD-1553739 | csusugcuGfgUfUfCfuccggcuguuL96 | 4935 | asAfscagCfcGfGfagaaCfcAfgcaagsgsc | 5070 | GCCUUGCUGGUUCUCCGGCUGUG | 5205 |
| AD-1553745 | gsgsuucuCfcGfGfCfugugugccuuL96 | 4936 | asAfsggcAfcAfCfagccGfgAfgaaccsasg | 5071 | CUGGUUCUCCGGCUGUGUGCCUG | 5206 |
| AD-1553751 | csuscgucAfcUfCfAfcgggcuguuuL96 | 4937 | asAfsacaGfcCfCfgugaGfuGfacgagsusg | 5072 | CACUCGUCACUCACGGGCUGUUG | 5207 |
| AD-1553758 | csuscacgGfgCfUfGfuugcuccaguL96 | 4938 | asCfsuggAfgCfAfacagCfcCfgugagsusg | 5073 | CACUCACGGGCUGUUGCUCCAGG | 5208 |
| AD-1553769 | ususgcucCfaGfGfCfcuggucucguL96 | 4939 | asCfsgagAfcCfAfggccUfgGfagcaascsa | 5074 | UGUUGCUCCAGGCCUGGUCUCGG | 5209 |
| AD-1553794 | gsgscucuCfaGfGfCfgcauuucucuL96 | 4940 | asGfsagaAfaUfGfcgccUfgAfgagccsgsg | 5075 | CCGGCUCUCAGGCGCAUUUCUCC | 5210 |
| AD-1553799 | uscsaggcGfcAfUfUfucuccgagcuL96 | 4941 | asGfscucGfgAfGfaaauGfcGfccugasgsa | 5076 | UCUCAGGCGCAUUUCUCCGAGCA | 5211 |
| AD-1553804 | csgscauuUfcUfCfCfgagcauccguL96 | 4942 | asCfsggaUfgCfUfcggaGfaAfaugcgscsc | 5077 | GGCGCAUUUCUCCGAGCAUCCGU | 5212 |
| AD-1553809 | ususcuccGfaGfCfAfuccgucuauuL96 | 4943 | asAfsuagAfcGfGfaugcUfcGfgagaasasu | 5078 | AUUUCUCCGAGCAUCCGUCUAUG | 5213 |
| AD-1553815 | gsasgcauCfcGfUfCfuaugggcaguL96 | 4944 | asCfsugcCfcAfUfagacGfgAfugcucsgsg | 5079 | CCGAGCAUCCGUCUAUGGGCAGU | 5214 |
| AD-1553820 | uscscgucUfaUfGfGfgcaguuuguuL96 | 4945 | asAfscaaAfcUfGfcccaUfaGfacggasusg | 5080 | CAUCCGUCUAUGGGCAGUUUGUG | 5215 |
| AD-1553826 | usasugggCfaGfUfUfuguggcugguL96 | 4946 | asCfscagCfcAfCfaaacUfgCfccauasgsa | 5081 | UCUAUGGGCAGUUUGUGGCUGGU | 5216 |
| AD-1553832 | csasguuuGfuGfGfCfuggugagacuL96 | 4947 | asGfsucuCfaCfCfagccAfcAfaacugscsc | 5082 | GGCAGUUUGUGGCUGGUGAGACA | 5217 |
| AD-1553839 | usgsgcugGfuGfAfGfacagcagaguL96 | 4948 | asCfsucuGfcUfGfucucAfcCfagccascsa | 5083 | UGUGGCUGGUGAGACAGCAGAGG | 5218 |
| AD-1553845 | gsusgagaCfaGfCfAfgaggagguguL96 | 4949 | asCfsaccUfcCfUfcugcUfgUfcucacscsa | 5084 | UGGUGAGACAGCAGAGGAGGUGA | 5219 |
| AD-1553852 | asgscagaGfgAfGfGfugaagggcuuL96 | 4950 | asAfsgccCfuUfCfaccuCfcUfcugcusgsu | 5085 | ACAGCAGAGGAGGUGAAGGGCUG | 5220 |
| AD-1553859 | gsasggugAfaGfGfGfcugcgugcauL96 | 4951 | asUfsgcaCfgCfAfgcccUfuCfaccucscsu | 5086 | AGGAGGUGAAGGGCUGCGUGCAG | 5221 |
| AD-1553865 | asasgggcUfgCfGfUfgcagcagcuuL96 | 4952 | asAfsgcuGfcUfGfcacgCfaGfcccuuscsa | 5087 | UGAAGGGCUGCGUGCAGCAGCUG | 5222 |
| AD-1553907 | usgscuggCfaGfUfGfcccacugaguL96 | 4953 | asCfsucaGfuGfGfgcacUfgCfcagcasgsu | 5088 | ACUGCUGGCAGUGCCCACUGAGG | 5223 |
| AD-1553912 | gscsagugCfcCfAfCfugaggaggauL96 | 4954 | asUfsccuCfcUfCfagugGfgCfacugcscsa | 5089 | UGGCAGUGCCCACUGAGGAGGAG | 5224 |
| AD-1553922 | csusgaggAfgGfAfGfccggacucuuL96 | 4955 | asAfsgagUfcCfGfgcucCfuCfcucagsusg | 5090 | CACUGAGGAGGAGCCGGACUCUG | 5225 |
| AD-1553930 | gsasgccgGfaCfUfCfugcugccaauL96 | 4956 | asUfsuggCfaGfCfagagUfcCfggcucscsu | 5091 | AGGAGCCGGACUCUGCUGCCAAG | 5226 |
| AD-1553937 | ascsucugCfuGfCfCfaagagugguuL96 | 4957 | asAfsccaCfuCfUfuggcAfgCfagaguscsc | 5092 | GGACUCUGCUGCCAAGAGUGGUG | 5227 |
| AD-1553942 | gscsugccAfaGfAfGfuggugaggcuL96 | 4958 | asGfsccuCfaCfCfacucUfuGfgcagcsasg | 5093 | CUGCUGCCAAGAGUGGUGAGGCG | 5228 |
| AD-1553951 | asgsugguGfaGfGfCfgugguaugauL96 | 4959 | asUfscauAfcCfAfcgccUfcAfccacuscsu | 5094 | AGAGUGGUGAGGCGUGGUAUGAG | 5229 |
| AD-1553957 | asasccucGfgUfGfCfuaugcugcguL96 | 4960 | asCfsgcaGfcAfUfagcaCfcGfagguuscsc | 5095 | GGAACCUCGGUGCUAUGCUGCGG | 5230 |
| AD-1553962 | csgsgugcUfaUfGfCfugcgguguguL96 | 4961 | asCfsacaCfcGfCfagcaUfaGfcaccgsasg | 5096 | CUCGGUGCUAUGCUGCGGUGUGU | 5231 |
| AD-1553967 | csusaugcUfgCfGfGfuguguggacuL96 | 4962 | asGfsuccAfcAfCfaccgCfaGfcauagscsa | 5097 | UGCUAUGCUGCGGUGUGUGGACC | 5232 |
| AD-1553975 | csgsguguGfuGfGfAfccugucacguL96 | 4963 | asCfsgugAfcAfGfguccAfcAfcaccgscsa | 5098 | UGCGGUGUGUGGACCUGUCACGG | 5233 |
| AD-1553988 | gsgsccagCfcUfCfAfugcagcugauL96 | 4964 | asUfscagCfuGfCfaugaGfgCfuggccsusc | 5099 | GAGGCCAGCCUCAUGCAGCUGAA | 5234 |
| AD-1553993 | gscscucaUfgCfAfGfcugaagguguL96 | 4965 | asCfsaccUfuCfAfgcugCfaUfgaggcsusg | 5100 | CAGCCUCAUGCAGCUGAAGGUGA | 5235 |
| AD-1553998 | asusgcagCfuGfAfAfggugacggcuL96 | 4966 | asGfsccgUfcAfCfcuucAfgCfugcausgsa | 5101 | UCAUGCAGCUGAAGGUGACGGCG | 5236 |
| AD-1554003 | gscsugaaGfgUfGfAfcggcgcugauL96 | 4967 | asUfscagCfgCfCfgucaCfcUfucagcsusg | 5102 | CAGCUGAAGGUGACGGCGCUGAC | 5237 |
| AD-1554009 | gsgsugacGfgCfGfCfugaccaguauL96 | 4968 | asUfsacuGfgUfCfagcgCfcGfucaccsusu | 5103 | AAGGUGACGGCGCUGACCAGUAC | 5238 |
| AD-1554018 | gscsugacCfaGfUfAfcucggcucuuL96 | 4969 | asAfsgagCfcGfAfguacUfgGfucagcsgsc | 5104 | GCGCUGACCAGUACUCGGCUCUG | 5239 |
| AD-1554025 | asgsuacuCfgGfCfUfcuguaaggauL96 | 4970 | asUfsccuUfaCfAfgagcCfgAfguacusgsg | 5105 | CCAGUACUCGGCUCUGUAAGGAG | 5240 |
| AD-1554030 | uscsggcuCfuGfUfAfaggagcuaguL96 | 4971 | asCfsuagCfuCfCfuuacAfgAfgccgasgsu | 5106 | ACUCGGCUCUGUAAGGAGCUAGC | 5241 |
| AD-1554036 | csusguaaGfgAfGfCfuagccucguuL96 | 4972 | asAfscgaGfgCfUfagcuCfcUfuacagsasg | 5107 | CUCUGUAAGGAGCUAGCCUCGUG | 5242 |
| AD-1554041 | asgsgagcUfaGfCfCfucgugggucuL96 | 4973 | asGfsaccCfaCfGfaggcUfaGfcuccususa | 5108 | UAAGGAGCUAGCCUCGUGGGUCA | 5243 |
| AD-1554048 | asgsccucGfuGfGfGfucagaaggcuL96 | 4974 | asGfsccuUfcUfGfacccAfcGfaggcusasg | 5109 | CUAGCCUCGUGGGUCAGAAGGCC | 5244 |
| AD-1554054 | gsusggguCfaGfAfAfggccaggaguL96 | 4975 | asCfsuccUfgGfCfcuucUfgAfcccacsgsa | 5110 | UCGUGGGUCAGAAGGCCAGGAGC | 5245 |
| AD-1554065 | gsgsccagGfaGfCfCfuccuuggaguL96 | 4976 | asCfsuccAfaGfGfaggcUfcCfuggccsusu | 5111 | AAGGCCAGGAGCCUCCUUGGAGC | 5246 |
| AD-1554070 | gsgsagccUfcCfUfUfggagcugaguL96 | 4977 | asCfsucaGfcUfCfcaagGfaGfgcuccsusg | 5112 | CAGGAGCCUCCUUGGAGCUGAGC | 5247 |
| AD-1554074 | gsasgaggCfuGfGfCfugaagcuauuL96 | 4978 | asAfsuagCfuUfCfagccAfgCfcucucsgsg | 5113 | CCGAGAGGCUGGCUGAAGCUAUG | 5248 |
| AD-1554082 | gsgscugaAfgCfUfAfuggacucuguL96 | 4979 | asCfsagaGfuCfCfauagCfuUfcagccsasg | 5114 | CUGGCUGAAGCUAUGGACUCUGG | 5249 |
| AD-1554089 | gscsuaugGfaCfUfCfugggcagaauL96 | 4980 | asUfsucuGfcCfCfagagUfcCfauagcsusu | 5115 | AAGCUAUGGACUCUGGGCAGAAC | 5250 |
| AD-1554097 | csuscuggGfcAfGfAfaccuccagguL96 | 4981 | asCfscugGfaGfGfuucuGfcCfcagagsusc | 5116 | GACUCUGGGCAGAACCUCCAGGU | 5251 |
| AD-1554106 | gsasaccuCfcAfGfGfucuccugccuL96 | 4982 | asGfsgcaGfgAfGfaccuGfgAfgguucsusg | 5117 | CAGAACCUCCAGGUCUCCUGCCU | 5252 |
| AD-1554111 | uscscaggUfcUfCfCfugccucaauuL96 | 4983 | asAfsuugAfgGfCfaggaGfaCfcuggasgsg | 5118 | CCUCCAGGUCUCCUGCCUCAAUG | 5253 |
| AD-1554119 | uscscugcCfuCfAfAfugcugagcauL96 | 4984 | asUfsgcuCfaGfCfauugAfgGfcaggasgsa | 5119 | UCUCCUGCCUCAAUGCUGAGCAG | 5254 |
| AD-1554126 | uscsaaugCfuGfAfGfcagaaccaguL96 | 4985 | asCfsuggUfuCfUfgcucAfgCfauugasgsg | 5120 | CCUCAAUGCUGAGCAGAACCAGC | 5255 |
| AD-1554131 | gscsugagCfaGfAfAfccagcaccuuL96 | 4986 | asAfsgguGfcUfGfguucUfgCfucagcsasu | 5121 | AUGCUGAGCAGAACCAGCACCUC | 5256 |
| AD-1554156 | gscsaucgGfgUfGfGfcacaguauguL96 | 4987 | asCfsauaCfuGfUfgccaCfcCfgaugcsasg | 5122 | CUGCAUCGGGUGGCACAGUAUGC | 5257 |
| AD-1554186 | csusccugGfuGfGfAfugcggaguauL96 | 4988 | asUfsacuCfcGfCfauccAfcCfaggagscsc | 5123 | GGCUCCUGGUGGAUGCGGAGUAC | 5258 |
| AD-1554193 | usgsgaugCfgGfAfGfuacaccucauL96 | 4989 | asUfsgagGfuGfUfacucCfgCfauccascsc | 5124 | GGUGGAUGCGGAGUACACCUCAC | 5259 |
| AD-1554198 | gscsggagUfaCfAfCfcucacugaauL96 | 4990 | asUfsucaGfuGfAfggugUfaCfuccgcsasu | 5125 | AUGCGGAGUACACCUCACUGAAC | 5260 |
| AD-1554203 | gsusacacCfuCfAfCfugaacccuguL96 | 4991 | asCfsaggGfuUfCfagugAfgGfuguacsusc | 5126 | GAGUACACCUCACUGAACCCUGC | 5261 |
| AD-1554210 | uscsacugAfaCfCfCfugcgcucucuL96 | 4992 | asGfsagaGfcGfCfagggUfuCfagugasgsg | 5127 | CCUCACUGAACCCUGCGCUCUCG | 5262 |
| AD-1554216 | asascccuGfcGfCfUfcucgcugcuuL96 | 4993 | asAfsgcaGfcGfAfgagcGfcAfggguuscsa | 5128 | UGAACCCUGCGCUCUCGCUGCUG | 5263 |
| AD-1554263 | cscscuggGfuGfUfGfgaacaccuauL96 | 4994 | asUfsaggUfgUfUfccacAfcCfcagggscsc | 5129 | GGCCCUGGGUGUGGAACACCUAC | 5264 |
| AD-1554268 | gsgsugugGfaAfCfAfccuaccagguL96 | 4995 | asCfscugGfuAfGfguguUfcCfacaccscsa | 5130 | UGGGUGUGGAACACCUACCAGGC | 5265 |
| AD-1554278 | ascscuacCfaGfGfCfcugucuaaauL96 | 4996 | asUfsuuaGfaCfAfggccUfgGfuaggusgsu | 5131 | ACACCUACCAGGCCUGUCUAAAG | 5266 |
| AD-1554287 | gscscuguCfuAfAfAfggacacauuuL96 | 4997 | asAfsaugUfgUfCfcuuuAfgAfcaggcscsu | 5132 | AGGCCUGUCUAAAGGACACAUUC | 5267 |
| AD-1554292 | uscsuaaaGfgAfCfAfcauucgagcuL96 | 4998 | asGfscucGfaAfUfguguCfcUfuuagascsa | 5133 | UGUCUAAAGGACACAUUCGAGCG | 5268 |
| AD-1554298 | gsgsacacAfuUfCfGfagcggcugguL96 | 4999 | asCfscagCfcGfCfucgaAfuGfuguccsusu | 5134 | AAGGACACAUUCGAGCGGCUGGG | 5269 |
| AD-1554317 | gsgsccuuCfgGfAfGfugaagcugguL96 | 5000 | asCfscagCfuUfCfacucCfgAfaggccsasg | 5135 | CUGGCCUUCGGAGUGAAGCUGGU | 5270 |
| AD-1554323 | csgsgaguGfaAfGfCfugguacgaguL96 | 5001 | asCfsucgUfaCfCfagcuUfcAfcuccgsasa | 5136 | UUCGGAGUGAAGCUGGUACGAGG | 5271 |
| AD-1554328 | usgsaagcUfgGfUfAfcgaggugcauL96 | 5002 | asUfsgcaCfcUfCfguacCfaGfcuucascsu | 5137 | AGUGAAGCUGGUACGAGGUGCAU | 5272 |
| AD-1554333 | csusgguaCfgAfGfGfugcauaucuuL96 | 5003 | asAfsgauAfuGfCfaccuCfgUfaccagscsu | 5138 | AGCUGGUACGAGGUGCAUAUCUG | 5273 |
| AD-1554340 | gsasggugCfaUfAfUfcuggacaaguL96 | 5004 | asCfsuugUfcCfAfgauaUfgCfaccucsgsu | 5139 | ACGAGGUGCAUAUCUGGACAAGG | 5274 |
| AD-1554346 | csasuaucUfgGfAfCfaaggagagauL96 | 5005 | asUfscucUfcCfUfugucCfaGfauaugscsa | 5140 | UGCAUAUCUGGACAAGGAGAGAG | 5275 |
| AD-1554351 | csusggacAfaGfGfAfgagagcgguuL96 | 5006 | asAfsccgCfuCfUfcuccUfuGfuccagsasu | 5141 | AUCUGGACAAGGAGAGAGCGGUG | 5276 |
| AD-1554371 | gscsccagCfuCfCfAfugggauggauL96 | 5007 | asUfsccaUfcCfCfauggAfgCfugggcscsa | 5142 | UGGCCCAGCUCCAUGGGAUGGAA | 5277 |
| AD-1554376 | gscsuccaUfgGfGfAfuggaagaccuL96 | 5008 | asGfsgucUfuCfCfauccCfaUfggagcsusg | 5143 | CAGCUCCAUGGGAUGGAAGACCC | 5278 |
| AD-1554379 | csuscagcCfuGfAfCfuaugaggccuL96 | 5009 | asGfsgccUfcAfUfagucAfgGfcugagsusg | 5144 | CACUCAGCCUGACUAUGAGGCCA | 5279 |
| AD-1554387 | gsascuauGfaGfGfCfcaccagucauL96 | 5010 | asUfsgacUfgGfUfggccUfcAfuagucsasg | 5145 | CUGACUAUGAGGCCACCAGUCAG | 5280 |
| AD-1554393 | gsasggccAfcCfAfGfucagaguuauL96 | 5011 | asUfsaacUfcUfGfacugGfuGfgccucsasu | 5146 | AUGAGGCCACCAGUCAGAGUUAC | 5281 |
| AD-1554399 | ascscaguCfaGfAfGfuuacagccguL96 | 5012 | asCfsggcUfgUfAfacucUfgAfcuggusgsg | 5147 | CCACCAGUCAGAGUUACAGCCGC | 5282 |
| AD-1554404 | uscsagagUfuAfCfAfgccgcugccuL96 | 5013 | asGfsgcaGfcGfGfcuguAfaCfucugascsu | 5148 | AGUCAGAGUUACAGCCGCUGCCU | 5283 |
| AD-1554413 | csasgccgCfuGfCfCfuggaacugauL96 | 5014 | asUfscagUfuCfCfaggcAfgCfggcugsusa | 5149 | UACAGCCGCUGCCUGGAACUGAU | 5284 |
| AD-1554418 | gscsugccUfgGfAfAfcugaugcuguL96 | 5015 | asCfsagcAfuCfAfguucCfaGfgcagcsgsg | 5150 | CCGCUGCCUGGAACUGAUGCUGA | 5285 |
| AD-1554425 | gsgsaacuGfaUfGfCfugacgcacguL96 | 5016 | asCfsgugCfgUfCfagcaUfcAfguuccsasg | 5151 | CUGGAACUGAUGCUGACGCACGU | 5286 |
| AD-1554430 | usgsaugcUfgAfCfGfcacguggccuL96 | 5017 | asGfsgccAfcGfUfgcguCfaGfcaucasgsu | 5152 | ACUGAUGCUGACGCACGUGGCCC | 5287 |
| AD-1554433 | csasugugCfcAfCfCfucauggugguL96 | 5018 | asCfscacCfaUfGfagguGfgCfacaugsgsg | 5153 | CCCAUGUGCCACCUCAUGGUGGC | 5288 |
| AD-1554443 | csuscaugGfuGfGfCfuucccacaauL96 | 5019 | asUfsuguGfgGfAfagccAfcCfaugagsgsu | 5154 | ACCUCAUGGUGGCUUCCCACAAU | 5289 |
| AD-1554451 | gsgscuucCfcAfCfAfaugaggaauuL96 | 5020 | asAfsuucCfuCfAfuuguGfgGfaagccsasc | 5155 | GUGGCUUCCCACAAUGAGGAAUC | 5290 |
| AD-1554456 | cscscacaAfuGfAfGfgaaucuguuuL96 | 5021 | asAfsacaGfaUfUfccucAfuUfgugggsasa | 5156 | UUCCCACAAUGAGGAAUCUGUUC | 5291 |
| AD-1554461 | asasugagGfaAfUfCfuguucgccauL96 | 5022 | asUfsggcGfaAfCfagauUfcCfucauusgsu | 5157 | ACAAUGAGGAAUCUGUUCGCCAG | 5292 |
| AD-1554470 | uscsuguuCfgCfCfAfggcaaccaauL96 | 5023 | asUfsuggUfuGfCfcuggCfgAfacagasusu | 5158 | AAUCUGUUCGCCAGGCAACCAAG | 5293 |
| AD-1554475 | uscsgccaGfgCfAfAfccaagcgcauL96 | 5024 | asUfsgcgCfuUfGfguugCfcUfggcgasasc | 5159 | GUUCGCCAGGCAACCAAGCGCAU | 5294 |
| AD-1554482 | gscsaaccAfaGfCfGfcaugugggauL96 | 5025 | asUfscccAfcAfUfgcgcUfuGfguugcscsu | 5160 | AGGCAACCAAGCGCAUGUGGGAG | 5295 |
| AD-1554487 | csasagcgCfaUfGfUfgggagcugguL96 | 5026 | asCfscagCfuCfCfcacaUfgCfgcuugsgsu | 5161 | ACCAAGCGCAUGUGGGAGCUGGG | 5296 |
| AD-1554492 | gscsauguGfgGfAfGfcugggcauuuL96 | 5027 | asAfsaugCfcCfAfgcucCfcAfcaugcsgsc | 5162 | GCGCAUGUGGGAGCUGGGCAUUC | 5297 |
| AD-1554497 | usgsggagCfuGfGfGfcauuccucuuL96 | 5028 | asAfsgagGfaAfUfgcccAfgCfucccascsa | 5163 | UGUGGGAGCUGGGCAUUCCUCUG | 5298 |
| AD-1554505 | gsgsgcauUfcCfUfCfuggaugggauL96 | 5029 | asUfscccAfuCfCfagagGfaAfugcccsasg | 5164 | CUGGGCAUUCCUCUGGAUGGGAC | 5299 |
| AD-1554510 | ususccucUfgGfAfUfgggacugucuL96 | 5030 | asGfsacaGfuCfCfcaucCfaGfaggaasusg | 5165 | CAUUCCUCUGGAUGGGACUGUCU | 5300 |
| AD-1554515 | csusggauGfgGfAfCfugucuguuuuL96 | 5031 | asAfsaacAfgAfCfagucCfcAfuccagsasg | 5166 | CUCUGGAUGGGACUGUCUGUUUC | 5301 |
| AD-1554520 | usgsggacUfgUfCfUfguuucggacuL96 | 5032 | asGfsuccGfaAfAfcagaCfaGfucccasusc | 5167 | GAUGGGACUGUCUGUUUCGGACA | 5302 |
| AD-1554525 | csusgucuGfuUfUfCfggacaacuuuL96 | 5033 | asAfsaguUfgUfCfcgaaAfcAfgacagsusc | 5168 | GACUGUCUGUUUCGGACAACUUC | 5303 |
| AD-1554533 | ususcggaCfaAfCfUfucugggcauuL96 | 5034 | asAfsugcCfcAfGfaaguUfgUfccgaasasc | 5169 | GUUUCGGACAACUUCUGGGCAUG | 5304 |
| AD-1554542 | csusucugGfgCfAfUfgugugaccauL96 | 5035 | asUfsgguCfaCfAfcaugCfcCfagaagsusu | 5170 | AACUUCUGGGCAUGUGUGACCAC | 5305 |
| AD-1554551 | asusguguGfaCfCfAfcgucucucuuL96 | 5036 | asAfsgagAfgAfCfguggUfcAfcacausgsc | 5171 | GCAUGUGUGACCACGUCUCUCUA | 5306 |
| AD-1554557 | gsasccacGfuCfUfCfucuagcacuuL96 | 5037 | asAfsgugCfuAfGfagagAfcGfuggucsasc | 5172 | GUGACCACGUCUCUCUAGCACUG | 5307 |
| AD-1554562 | gscsaggcCfgGfCfUfauguaguguuL96 | 5038 | asAfscacUfaCfAfuagcCfgGfccugcscsc | 5173 | GGGCAGGCCGGCUAUGUAGUGUA | 5308 |
| AD-1554567 | cscsggcuAfuGfUfAfguguauaaguL96 | 5039 | asCfsuuaUfaCfAfcuacAfuAfgccggscsc | 5174 | GGCCGGCUAUGUAGUGUAUAAGU | 5309 |
| AD-1554573 | asusguagUfgUfAfUfaaguccauuuL96 | 5040 | asAfsaugGfaCfUfuauaCfaCfuacausasg | 5175 | CUAUGUAGUGUAUAAGUCCAUUC | 5310 |
| AD-1554578 | gsusguauAfaGfUfCfcauucccuauL96 | 5041 | asUfsaggGfaAfUfggacUfuAfuacacsusa | 5176 | UAGUGUAUAAGUCCAUUCCCUAU | 5311 |
| AD-1554584 | asasguccAfuUfCfCfcuauggcucuL96 | 5042 | asGfsagcCfaUfAfgggaAfuGfgacuusasu | 5177 | AUAAGUCCAUUCCCUAUGGCUCC | 5312 |
| AD-1554591 | ususcccuAfuGfGfCfuccuuggaguL96 | 5043 | asCfsuccAfaGfGfagccAfuAfgggaasusg | 5178 | CAUUCCCUAUGGCUCCUUGGAGG | 5313 |
| AD-1554599 | gsgscuccUfuGfGfAfggagguaauuL96 | 5044 | asAfsuuaCfcUfCfcuccAfaGfgagccsasu | 5179 | AUGGCUCCUUGGAGGAGGUAAUC | 5314 |
| AD-1554609 | asusccggAfgGfGfCfccaggagaauL96 | 5045 | asUfsucuCfcUfGfggccCfuCfcggauscsa | 5180 | UGAUCCGGAGGGCCCAGGAGAAC | 5315 |
| AD-1554626 | gsasaccgGfaGfCfGfugcuucagguL96 | 5046 | asCfscugAfaGfCfacgcUfcCfgguucsusc | 5181 | GAGAACCGGAGCGUGCUUCAGGG | 5316 |
| AD-1554642 | csasggguGfcCfCfGfcagggaacauL96 | 5047 | asUfsguuCfcCfUfgcggGfcAfcccugsasa | 5182 | UUCAGGGUGCCCGCAGGGAACAG | 5317 |
| AD-1554653 | csasgggaAfcAfGfGfagcugcucauL96 | 5048 | asUfsgagCfaGfCfuccuGfuUfcccugscsg | 5183 | CGCAGGGAACAGGAGCUGCUCAG | 5318 |
| AD-1554658 | asascaggAfgCfUfGfcucagccaauL96 | 5049 | asUfsuggCfuGfAfgcagCfuCfcuguuscsc | 5184 | GGAACAGGAGCUGCUCAGCCAAG | 5319 |
| AD-1554663 | gsasgcugCfuCfAfGfccaagaacuuL96 | 5050 | asAfsguuCfuUfGfgcugAfgCfagcucscsu | 5185 | AGGAGCUGCUCAGCCAAGAACUG | 5320 |
| AD-1554668 | gscsucagCfcAfAfGfaacuguggcuL96 | 5051 | asGfsccaCfaGfUfucuuGfgCfugagcsasg | 5186 | CUGCUCAGCCAAGAACUGUGGCG | 5321 |
| AD-1554690 | usgsccagGfaUfGfCfcgaaggauauL96 | 5052 | asUfsaucCfuUfCfggcaUfcCfuggcasgsc | 5187 | GCUGCCAGGAUGCCGAAGGAUAC | 5322 |
| AD-1554696 | uscsauguGfgUfCfAfauaaaagucuL96 | 5053 | asGfsacuUfuUfAfuugaCfcAfcaugascsc | 5188 | GGUCAUGUGGUCAAUAAAAGUCC | 5323 |
| AD-1554704 | uscsaauaAfaAfGfUfccuuagguguL96 | 5054 | asCfsaccUfaAfGfgacuUfuUfauugascsc | 5189 | GGUCAAUAAAAGUCCUUAGGUGC | 5324 |
| AD-1554709 | asasaaguCfcUfUfAfggugcugccuL96 | 5055 | asGfsgcaGfcAfCfcuaaGfgAfcuuuusasu | 5190 | AUAAAAGUCCUUAGGUGCUGCCU | 5325 |

Example 2. A Randomized, Double-Blind, Placebo-Controlled Study to Evaluate the Efficacy, Safety, Pharmacodynamics, and Pharmacokinetics of Lumasiran in Patients with Recurrent Calcium Oxalate Kidney Stone Disease and Elevated Urinary Oxalate Levels

Kidney stones are common, affecting approximately 1 in 11 people in the United States, and the prevalence of kidney stone disease has been increasing worldwide over time (Scales, et al. Eur Urol. 2012 July; 62(1):160-5). Approximately 80% of kidney stones in adults are formed from calcium oxalate crystals, with the remainder being predominantly calcium phosphate, uric acid, cystine, or struvite (Worcester E M and Coe F L. Nephrolithiasis. Prim Care. 2008 June; 35(2):369-91; Worcester E M, Coe F L. N Engl J Med. 2010 Sep. 2; 363(10):954-63). Stone formation occurs when a supersaturating level of calcium oxalate is present in the urine, with increasing risk of stone formation as urine oxalate levels increase (Curhan and Taylor Kidney Int. 2008 February; 73(4):489-96). High levels of urinary oxalate may be derived from both endogenous synthesis and diet. More than half of oxalate is endogenous in origin and presumed to come largely from the liver (Mitchell, et al. Circ Res. 2018 Feb. 16; 122(4):555-9). Studies have shown that reduced calcium oxalate supersaturation and urinary oxalate levels are associated with reduced stone formation (Borgh, et al. N Engl J Med. 2002 Jan. 10; 346(2):77-84; Ferrar, et al. J Urol. 2018 November; 200(5):1082-7; Prochaska, et al. J Urol. 2018 May; 199(5):1262-6].

Kidney stones can develop in patients of all ages; however, the highest incidence rates occur in individuals aged 40 to 66 years (Shin, et al. World J Nephrol. 2018 Nov. 24; 7(7):129-42). There is significant clinical burden associated with the development of kidney stones for patients with recurrent calcium oxalate kidney stone disease, including pain, infection/sepsis, diagnostic and therapeutic procedures, hospitalizations, and a greater risk for developing chronic kidney disease (CKD) and end stage kidney disease (ESKD). For patients with recurrent calcium oxalate stone formation, multiple stone removal procedures may be required. These procedures are invasive and place the patient at risk of complications including bleeding and infection. Patients experiencing obstructive kidney stones can also experience acute kidney injury with permanent loss of renal function. As a result, patients with recurrent kidney stone formation have a higher risk of progression to CKD and ESKD (Dhondup, et al. Am J Kidney Dis. 2018 December; 72(6):790-72018; Rul, et al. Clin J Am Soc Nephrol. 2009 April; 4(4):804-11).

The typical clinical presentation of kidney stones includes sudden onset of lumbar flank pain and hematuria, and may include nausea and vomiting. Evaluation to assess etiology includes assessment of the patient's medical history, medication use, and dietary and lifestyle risk factors. Confirmation of diagnosis may involve renal ultrasound, abdominal x-ray, and/or computed tomography (CT) Heilberg, et al. Endocrinol Metabol. 2006 August; 50(4):823-31). Twenty-four-hour urine collections analyzed for total volume, calcium, oxalate, uric acid, citrate, and other analytes may help to determine the underlying etiology (Pearle, et al. J Urol. 2014 August; 192(2):316-24). Stone composition is generally determined in at least one instance.

There are limited effective treatment options for patients with recurrent calcium oxalate kidney stone disease. Preventive measures in American and European guidelines recommend adequate fluid intake to ensure a urine volume of at least 2 to 2.5 liters daily and provide dietary advice to limit the consumption of oxalate-rich foods, sodium chloride, and animal protein content, while maintaining a normal calcium intake. In some situations, thiazide diuretics, potassium citrate, and/or allopurinol may be considered (Pearle, supra; Turk, et al. EAU Guidelines on Urolithiasis. EAU Annual Congress; 2021; Milan, Italy: EAU Guidelines Office).

Treatment of pain associated with kidney stone events may involve non-steroidal anti-inflammatory agents and/or opiate pain medications. Depending on the clinical context, medical expulsive therapy, extracorporeal shock-wave lithotripsy, ureteroscopy, stenting, and percutaneous nephrolithotomy are some of the treatment options that may be pursued (Turk, supra).

Lumasiran is a ribonucleic acid interference (RNAi) therapeutic that target glycolate oxidase (GO, or HAO1) which reduces hepatic oxalate production. Oxalate produced by the liver is largely excreted in the urine, and lumasiran has been shown to reduce urinary oxalate in patients with PH1. High levels of urinary oxalate increase the risk of stone formation; therefore, lumasiran may have efficacy in patients with recurrent calcium oxalate kidney stone disease who do not have PH1 but who produce high amounts of oxalate endogenously.

The sense strand of lumasiran comprises the nucleotide sequence nucleotide sequence 5′-gsascuuuCfaUfCfCfuggaaauaua-3′ (SEQ ID NO:35) and the antisense strand comprises the nucleotide sequence 5′-usAfsuauUfuCfCfaggaUfgAfaagucscsa-3′ (SEQ ID NO:36). The sense strand of lumasiran is conjugated to a ligand as shown in the following schematic

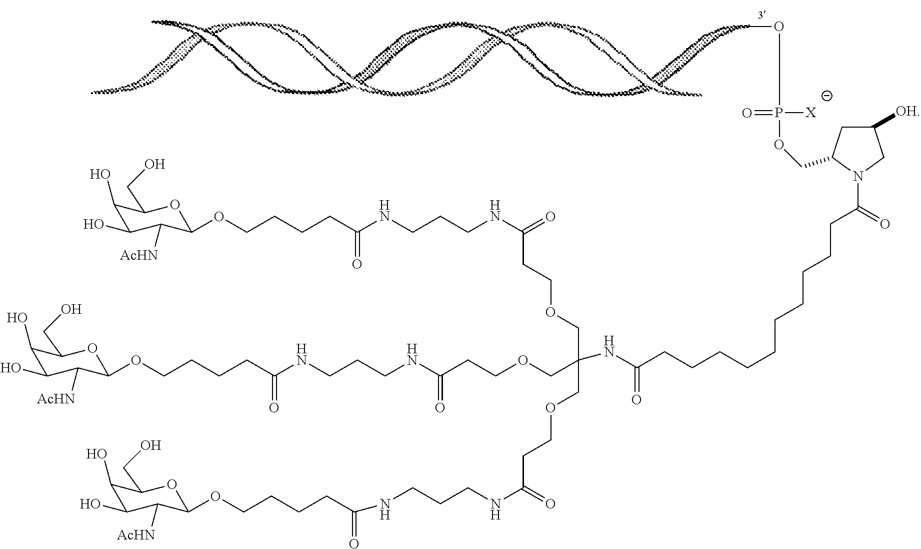

and, wherein X is O.

### Summary of Study Design

The study is a randomized, placebo-controlled, double-blind, multicenter, multinational, Phase 2 study to evaluate the efficacy, safety, pharmacodynamics (PD), and pharmacokinetics (PK) of lumasiran administered subcutaneously (SC) in patients with recurrent calcium oxalate kidney stone disease and elevated urinary oxalate levels.

The study consists of up to 2 months of screening and 15 months of double-blind treatment (a 6-month Primary Analysis Period followed by a 9-month Treatment Extension Period). Patients were screened from Day −60 to Day −1 to determine eligibility. During screening, patients provided at least two 24-hour urine collections to establish baseline urinary oxalate levels. Consented patients meeting all eligibility criteria were randomized 1:1:1 to receive study drug: lumasiran 567 mg, lumasiran 284 mg, or placebo. Stratification was performed at randomization according to mean baseline urinary oxalate level and the number of kidney stone events in the 12 months prior to screening.

Patients were administered SC injections of lumasiran (284 mg or 567 mg) and/or placebo at the same volume (1.5. mL), on Day 1, Month 3, and Month 9.

| Lumasiran 567 mg | Lumasiran 284 mg | Placebo |
|---|---|---|
| 1.5 mL lumasiran | 1.5 mL lumasiran | 1.5 mL placebo |
| 1.5 mL lumasiran | 1.5 mL placebo | 1.5 mL placebo |

During the 6-month Primary Analysis Period, patients were dosed on Day 1 (baseline) and at Month 3. During the Treatment Extension Period, one additional dose will be administered at Month 9; an end of study (EOS) visit will take place at Month 15. Study drug was administered SC. Patients will be assessed for efficacy, safety, PD, and PK. Efficacy assessments will include evaluation of urinary oxalate excretion, urinary calcium oxalate supersaturation, and kidney stone events (including clinical events and low-dose kidney-protocol CT). Safety assessments will include collection of adverse events (AEs), clinical laboratory tests, vital sign assessments, physical examinations, and concomitant medications.

### Rationale for Study Design

The primary endpoint for this Phase 2 study is the percent change in 24-hour urinary oxalate excretion. To confirm the optimal dosing regimen, and to facilitate the collection of kidney stone event data (an exploratory endpoint), the study will continue through Month 15. A placebo comparator is included because there is no approved standard of care therapy to decrease urinary oxalate.

A blood DNA sample will be collected as part of standard screening assessments (if testing has not already been performed) to ensure the exclusion of patients with primary hyperoxaluria type 1 (PH1), type 2 (PH2), and type 3 (PH3). Lumasiran is approved in some countries for the treatment of PH1, and patients with PH2 and PH3 are not expected to respond to lumasiran.

Because the primary endpoint will rely on measurements of urinary oxalate, and because some urinary oxalate is diet-derived, diet is an important variable in this study. In a 5-year study of recurrent stone formers published by Borghi et al. (supra), patients randomized to a normal calcium, low protein/salt diet had lower urinary oxalate levels and a lower cumulative incidence of recurrent kidney stones when compared to a low calcium diet. During the current study, and as of the time of informed consent, patients will be asked to adhere to a diet appropriate for stone formers, including adequate calcium intake and avoidance of spinach and other foods that are high in oxalate.

The secondary endpoint to assess meaningful reduction in 24-hour urinary oxalate from baseline to Month 6 (Months 4 through 6) defines a clinically meaningful reduction as ≥20% in the non-PH1 stone former population, supported by available literature based on stone former populations.

### Treatment Groups

Patients were randomized 1:1:1 to receive lumasiran 284 mg, lumasiran 567 mg, or placebo, administered at the same volume, for the duration of the study. Stratification was performed at randomization according to mean baseline urinary oxalate level (>1.25×ULN vs ≤1.25×ULN) and the number of historical kidney stone events in the 12 months prior to screening (>1 vs ≤1).

For stratification, a historical kidney stone event is defined as:
the visible passage of a kidney stone
a procedural intervention for removal of an asymptomatic or symptomatic stone
   if more than 1 stone was removed in a given procedure, this counts as 1 event unless bilateral ureteral stones were removed, in which case this counts as 2 events
   if more than 1 procedure was required to remove a single stone, this counts as 1 event
a new (≥1 mm) or enlarged (by ≥2 mm) kidney stone on CT imaging
   it must be evident from the CT scans that the new or enlarged kidney stone event occurred during the 12 months prior to screening
   if a procedure was performed to remove the stone(s) identified by CT, then only the procedure will be counted to avoid double-counting the same stone.

### Inclusion Criteria

Patients are eligible to be included in the study if all the following criteria apply:

### Age

1. Age 18 years or older (or age of legal consent, whichever is older).

### Patient and Disease Characteristics

2. Recurrent kidney stone disease, defined as ≥2 stone events within the 5 years prior to screening. For inclusion, a historical kidney stone event is defined as:
the visible passage of a kidney stone
a procedural intervention for removal of an asymptomatic or symptomatic stone
   if more than 1 stone was removed in a given procedure, this counts as 1 event unless bilateral ureteral stones were removed, in which case this counts as 2 events
   if more than 1 procedure was required to remove a single stone, this counts as 1 event
a new (≥1 mm) or enlarged (by ≥2 mm) kidney stone on CT imaging
   it must be evident from the CT scans that the new or enlarged kidney stone event occurred during 5 years prior to screening
   if a procedure was performed to remove the stone(s) identified by CT, then only the procedure will be counted to avoid double-counting the same stone.
      3. The 2 most recently analyzed kidney stones prior to randomization contained 50% or more of calcium oxalate; if only one stone analysis is available, then it must have contained 50% or more of calcium oxalate.
      4. 24-hour urinary oxalate levels from 2 valid 24-hour urine collections obtained during screening are >ULN.
      5. Willing to adhere to dietary recommendations appropriate for stone formers including limiting vitamin C supplementation to <200 mg daily.
      6. If taking medications and/or hydrating for kidney stone prophylaxis, or taking medications that alter urinary oxalate excretion and/or kidney stone formation, must have been on a stable regimen for at least 60 days before randomization, and willing to remain on this stable regimen for the duration of the study.
      7. Body mass index (the weight in kilograms divided by the square of the height in meters) of 20 to <40 kg/m².

### Exclusion Criteria

Patients are excluded from the study if any of the following criteria apply:

### Laboratory Assessments

1. Has any of the following laboratory parameter assessments at screening:
a. Alanine aminotransferase (ALT) or aspartate aminotransferase (AST)>2×ULN
b. Total bilirubin >1.5×ULN. Patients with elevated total bilirubin that is secondary to documented Gilbert's syndrome are eligible if the total bilirubin is <2×ULN
c. International normalized ratio (INR) ≥2.0 (patients on oral anticoagulant [eg, warfarin] with an INR <3.5 will be allowed)
   2. Has an eGFR of <30 mL/min/1.73 m² at screening (calculation will be based on the Chronic Kidney Disease Epidemiology Collaboration [CKD-EPI] creatinine formula).

### Prior/Concomitant Therapy

3. Received an investigational agent within the last 30 days or 5 half-lives, whichever is longer, prior to the first dose of study drug, or are in follow-up of another clinical study prior to study enrollment. Any agent that has received health agency authorization (including for emergency use) by local or regional regulatory authorities is not considered investigational.

### Medical Conditions

4. Patients with a known history of secondary causes of elevated urinary oxalate and/or recurrent kidney stones including:
a. Primary hyperoxaluria
b. Severe eating disorders (anorexia or bulimia)
c. Chronic inflammatory bowel disease
d. Intestinal surgery with malabsorption or chronic diarrhea
e. Sarcoidosis
f. Primary hyperparathyroidism
g. Complete distal renal tubular acidosis
   5. Has other medical conditions or comorbidities which, in the opinion of the Investigator, would interfere with study compliance or data interpretation.
   6. History of multiple drug allergies or history of allergic reaction to an oligonucleotide or GalNAc.
   7. History of intolerance to SC injection(s).

### Contraception, Pregnancy, and Breastfeeding

8. Is not willing to comply with the contraceptive requirements during the study period.
9. Female patient is pregnant, planning a pregnancy, or breast-feeding.

### Alcohol Use

10. Unwilling or unable to limit alcohol consumption throughout the course of the study. Alcohol intake of >2 units/day is excluded during the study (unit: 1 glass of wine [approximately 125 mL]=1 measure of spirits [approximately 1 fluid ounce]=½ pint of beer [approximately 284 mL]).
11. History of alcohol abuse, within the last 12 months before screening, in the opinion of the Investigator.

### Efficacy Assessments

### 24-Hour Urine Collections

Urinary oxalate excretion and calcium oxalate supersaturation (calculated from multiple parameters) will be determined from 24-hour urine sample collections to be completed at the time points specified. The start and stop dates/times of collection, the volume of urine in the collection, whether there were any missed voids, and whether the patient complied with dietary recommendations will be recorded. An aliquot of the 24-hour urine collection will also be used to determine urinary creatinine content and to determine if the 24-hour urine collections need to be repeated.

### Validity Criteria for 24-Hour Urine Collections

Throughout the study, a urine collection will be considered valid if each of the following criteria are met:
The collection is between 22 to 26 hours in duration between the initial discarded void and the last void or attempt to void.
No voids are missed between the start and end time of the collection as indicated by the patient's urine collection diary.
The 24-hour creatinine content is at least 10 mg/kg as assessed by the central laboratory.
Patient complied with dietary recommendations appropriate for oxalate stone formers (detailed in the Dietary Reference Sheet) for the 4 days prior to the start of the urine collection and during the collection.
24-hour urine collections that are known to be invalid should still be submitted for analysis.

### Variability Criterion for 24-Hour Urine Collections at Screening

If the 2 valid 24-hour urine collections from screening meet eligibility requirements (both 24-hour urinary oxalate levels >ULN), the variability between the oxalate levels (in mg/day) should be assessed as follows:

$$Variability = \vert \frac{( Oxalate value \#1 - Oxalate value \#2 )}{( Average of oxalate values \#1 \& \#2 )} \vert \times 100 \%$$

If the variability is >20%, then a third valid 24-hour urine collection should be obtained. The result of the third sample will not impact the patient's eligibility for the study.

### Kidney Stone Events

Since kidney stone events are recorded as an efficacy assessment, these events will not be captured as AEs or serious adverse events (SAEs). However, if a patient experiences other AEs or SAEs during a kidney stone event, they should be reported as an AE.

Kidney stone events will be graded by the Investigator as mild, moderate, or severe:

| | |
|---|---|
| Mild: | Mild; asymptomatic or mild symptoms; clinical or diagnostic |
| | observations only; intervention not indicated. |
| Mod- | Moderate; minimal, local or noninvasive intervention indicated; |
| erate: | limiting age appropriate instrumental activities of daily living |
| | (eg, preparing meals, shopping for groceries or clothes, using the |
| | telephone, managing money). |
| Severe: | Severe or medically significant but not immediately life- |
| | threatening; hospitalization or prolongation of hospitalization |
| | indicated; disabling; limiting self-care activities of daily living |
| | (ie, bathing, dressing and undressing, feeding self, using the |
| | toilet, taking medications, and not bedridden); OR life- |
| | threatening consequences; urgent intervention indicated; OR |
| | death related to an AE. |

If there are changes in grade during an event, only the highest grade should be reported.

### Clinical

All relevant clinical information pertaining to a kidney stone events should be obtained, including laboratory values, medical records, discharge summaries, and medical test results (including stone composition, if available, and radiology reports). A clinical kidney stone event is defined as one of the following:
Visible passage of a kidney stone
A procedural intervention for removal of an asymptomatic or symptomatic stone (information on the location, number, and size of stones removed will be collected)
Or, in the case of potential stone passages without visible stones, it will be up to the Investigator to evaluate patients' symptoms and determine whether a stone passage occurred or the symptoms were due to a different cause.

### Radiographic

A non-contrast low-dose kidney-protocol CT scan will be performed for all patients on Day 1 (may be performed up to 3 days prior to Day 1), and at Month 15.

For patients who terminate the study early, a CT scan should be performed at the ET visit only if this visit occurs after Month 6 and at the discretion of the Investigator, and where permitted, following consultation with the Medical Monitor. CT scans will be analyzed centrally.

### Spot Urinary Oxalate:Creatinine Ratios

Urine oxalate:creatinine ratios will be calculated from the oxalate and creatinine levels measured in single-void urine collections. Single-void urine collections should be collected as a first morning void when possible; if this is not possible then the reason should be documented.

### Estimated Glomerular Filtration Rate

Blood samples for the assessment of eGFR (mL/min/1.73 m²) will be obtained at the time points specified.

eGFR will be calculated based on the CKD-EPI formula:

CKD-EPI (Chronic Kidney Disease Epidemiology Collaboration). A new equation to estimate glomerular filtration rate. Ann Intern Med. 2009 May 5; 150(9):604-12.
Conventional units

$$eGFR ( mL / min / 1.73 {m}^{2} ) = 175 \times {( S C r [ mg / dL ] )}^{- 1.154} \times {( age )}^{- 0.203} \times ( 0.742 , if female ) ,$$

$$or \times ( 1.212 , if African American )$$

SI units

$$eGFR ( mL / min / 1.73 {m}^{2} ) = 175 \times {( SCr [ μmg / L ] / 88.4 )}^{- 1.154} \times {( age )}^{- 0.203} \times ( 0.742 , if female ) ,$$

$$or \times ( 1.212 , if African American )$$

Abbreviations: eGFR=Estimated glomerular filtration rate; SCr=serum creatinine; SI=International System of Units

### Pharmacodynamic Assessments

Urine and blood samples will be collected for assessment of PD parameters (plasma oxalate, plasma glycolate, and urinary glycolate) at the time points specified. Urine samples for exploratory analysis will be aliquoted from the samples provided for PD analysis. On dosing days, all blood and urine samples will be collected prior to study drug administration.

All PD assessments will be analyzed centrally. Postdose PD results will not be distributed to the sites until after the last patient completes assessments at the Month 15 visit. Site personnel should refrain from obtaining or viewing local oxalate, calcium oxalate supersaturation, or glycolate assessments, except as medically indicated, due to risk of unblinding.

Where local regulations allow and infrastructure is in place, a healthcare professional may collect urine or blood samples offsite.

### Pharmacokinetic Assessments

Blood samples will be collected for the assessment of lumasiran PK parameters at the time points indicated.

The concentration of lumasiran in blood samples will be determined using a validated assay.

### Safety Assessments

The assessment of safety during the study will consist of the surveillance and recording of AEs including SAEs, recording of concomitant medication and measurements of vital signs, weight and height, and laboratory tests. Clinically significant abnormalities observed during the physical examination are recorded as either medical history or AEs, as appropriate.

Safety assessments are to be performed as specified. On dosing days and as applicable, assessments of vital signs, weight/height, physical examination, and clinical laboratory assessments are to be completed before study drug administration.

### Quality of Life Outcomes

For pain assessments, patients will be asked to assess their “worst daily pain” (0=no pain at all; 10=pain as bad as you can imagine) from Question 3 of the Brief Pain Inventory—Short Form. This will be administered at screening, on Day 1, and daily while experiencing stone-related pain until the conclusion of the associated stone event.

The Wisconsin Stone Quality of Life Questionnaire (WISQOL) will be administered on Day 1 and upon conclusion of each clinical kidney stone event and will assess the degree of kidney stone impacts in terms of:
Fatigue
Sleep
Social function
Daily activities
Physical/psychosocial symptoms

### EQUIVALENTS

Those skilled in the art will recognize, or be able to ascertain using no more than routine experimentation, many equivalents to the specific embodiments and methods described herein. Such equivalents are intended to be encompassed by the scope of the following claims.

1. A method for inhibiting the expression of hydroxyacid oxidase (HAO1) in a human subject having a non-primary hyperoxaluria disease or disorder that would benefit from reduction in urinary oxalate, comprising
administering to the subject a fixed dose of about 200 mg to about 600 mg of a double stranded ribonucleic acid (dsRNA) agent, or salt thereof, which inhibits the expression of of HAO1,
thereby inhibiting the expression of HAO1 in the subject.

2. A method for reducing urinary oxalate levels in a human subject having a non-primary hyperoxaluria disease or disorder that would benefit from reduction in urinary oxalate, comprising
administering to the subject a fixed dose of about 200 mg to about 600 mg of a double stranded ribonucleic acid (dsRNA) agent, or salt thereof, which inhibits the expression of of HAO1,
thereby reducing urinary oxalate levels in the subject.

3. The method of claim 2, wherein the urinary oxalate is urinary calcium oxalate.

4. The method of claim 3, wherein the reduction in urinary calcium oxalate is reduction in urinary calcium oxalate supersaturation.

5. (canceled)

6. The method of claim 1, wherein the non-primary hyperoxaluria disease or disorder is selected from the group consisting of secondary hyperoxaluria, a kidney stone disease, chronic kidney disease (CKD), end-stage renal disease (ESRD), coronary artery disease, cutaneous oxalate deposition, ethylene glycol poisoning, planned kidney transplantation, and previous kidney transplantation.

7. The method of claim 6, wherein the non-primary hyperoxaluria disease or disorder is a kidney stone disease.

8. The method of claim 7, wherein the kidney stone disease is calcium oxalate kidney stone disease.

9. The method of claim 8, wherein the calcium oxalate kidney stone disease is recurrent calcium oxalate kidney stone disease.

10.-18. (canceled)

19. The method of claim 1, wherein the dsRNA agent, or salt thereof, is administered to the subject subcutaneously.

20. (canceled)

21. The method of claim 1, wherein the dsRNA agent, or salt thereof, comprises a sense strand and an antisense strand forming a double stranded region, wherein the sense strand comprises a nucleotide sequence comprising at least 15 contiguous nucleotides differing by no more than 3 nucleotides from a portion of the nucleotide sequence of SEQ ID NO: 21 and the antisense strand comprises a nucleotide sequence comprising at least 15 contiguous nucleotides differing by no more than 3 nucleotides from the corresponding portion of nucleotide sequence of SEQ ID NO: 22 such that the sense strand is complementary to the at least 15 contiguous nucleotides in the antisense strand.

22. (canceled)

23. The method of claim 1, wherein the dsRNA agent, or salt thereof, comprises a sense strand and an antisense strand forming a double-stranded region, wherein the sense strand comprises a nucleotide sequence differing by no more than 3 nucleotides from the nucleotide sequence 5′-GACUUUCAUCCUGGAAAUAUA-3′ (SEQ ID NO:33) and the antisense strand comprises a nucleotide sequence differing by no more than 3 nucleotides from the nucleotide sequence 5′-UAUAUUUCCAGGAUGAAAGUCCA-3′ (SEQ ID NO:34).

24.-26. (canceled)

27. The method of claim 1, wherein all of the nucleotides of the sense strand and all of the nucleotides of the antisense strand comprise a nucleotide modification.

28. The method of claim 27, wherein at least one of the nucleotide modifications is selected from the group a deoxy-nucleotide modification, a 3′-terminal deoxy-thymine (dT) nucleotide modification, a 2′-O-methyl nucleotide modification, a 2′-fluoro nucleotide modification, a 2′-deoxy-nucleotide modification, a locked nucleotide modification, an unlocked nucleotide modification, a conformationally restricted nucleotide modification, a constrained ethyl nucleotide modification, an abasic nucleotide modification, a 2′-amino-nucleotide modification, a 2′-O-allyl-nucleotide modification, 2′-C-alkyl-nucleotide modification, 2′-hydroxyl-nucleotide modification, a 2′-methoxyethyl nucleotide modification, a 2′-O-alkyl-nucleotide modification, a morpholino nucleotide modification, a phosphoramidate, a non-natural base comprising nucleotide modification, a tetrahydropyran nucleotide modification, a 1,5-anhydrohexitol modified nucleotide modification, a cyclohexenyl nucleotide modification, a nucleotide comprising a 5′-phosphorothioate group modification, a nucleotide comprising a 5′-methylphosphonate group modification, a nucleotide comprising a 5′ phosphate or 5′ phosphate mimic modification, a nucleotide comprising vinyl phosphonate modification, a nucleotide comprising adenosine-glycol nucleic acid (GNA) modification, a nucleotide comprising thymidine-glycol nucleic acid (GNA) S-Isomer modification, a nucleotide comprising 2-hydroxymethyl-tetrahydrofurane-5-phosphate modification, a nucleotide comprising 2′-deoxythymidine-3′phosphate modification, a nucleotide comprising 2′-deoxyguanosine-3′-phosphate modification, and a terminal nucleotide linked to a cholesteryl derivative modification and a dodecanoic acid bisdecylamide group modification; and combinations thereof.

29. The method of claim 27, wherein the dsRNA agent, or salt thereof, further comprises at least one phosphorothioate internucleotide linkage.

30.-33. (canceled)

34. The method of claim 1, wherein at least one strand of the dsRNA agent, or salt thereof, further comprises a ligand.

35. The method of claim 34, wherein the ligand is attached to the 3′ end of the sense strand.

36. The method of claim 34, wherein the ligand is one or more N-acetylgalactosamine (GalNAc) derivatives.

37.-79. (canceled)

80. A method for treating a subject having a non-primary hyperoxaluria disease or disorder that would benefit from reduction in oxalate, the method comprising
administering to the subject a therapeutically effective amount of a nucleic acid inhibitor of hydroxyacid oxidase (HAO1) and/or a nucleic acid inhibitor of Proline Dehydrogenase 2 (PRODH2),
thereby treating the subject having the non-primary hyperoxaluria disease or disorder that would benefit from reduction in oxalate.

81. The method of claim 80, wherein the non-primary hyperoxaluria disease or disorder is selected from the group consisting of secondary hyperoxaluria, a kidney stone disease, chronic kidney disease (CKD), end-stage renal disease (ESRD), coronary artery disease, cutaneous oxalate deposition, ethylene glycol poisoning, planned kidney transplantation, and previous kidney transplantation.

82. A method for treating a subject at risk of developing a non-primary hyperoxaluria disease or disorder that would benefit from reduction in oxalate, the method comprising
administering to the subject a therapeutically effective amount of a nucleic acid inhibitor of lactate dehydrogenase A (LDHA), a nucleic acid inhibitor of hydroxyacid oxidase (HAO1), and/or a nucleic acid inhibitor of Proline Dehydrogenase 2 (PRODH2),
thereby treating the subject at risk of developing the non-primary hyperoxaluria disease or disorder that would benefit from reduction in oxalate.

83-157. (canceled)
